(19) United States
(12) Patent Application Publication
(10) Pub. No.: US 2022/0306659 A1
(43) Pub. Date: Sep. 29, 2022

(54) COMPOUND INHIBITING AND INDUCING DEGRADATION OF EGFR AND ALK

(71) Applicant: BEIJING TIDE PHARMACEUTICAL CO., LTD., Beijing (CN)

(72) Inventors: Yanping Zhao, Beijing (CN); Hongjun Wang, Beijing (CN); Yeming Wang, Beijing (CN); Futian Fan, Beijing (CN); Yuanyuan Jiang, Beijing (CN); Xiaoqian Wang, Beijing (CN); Huining Liang, Beijing (CN); Yong Xie, Beijing (CN); Yanhao Zhang, Beijing (CN); Kai Liu, Beijing (CN); Zewang Feng, Beijing (CN); Xuelian Liu, Beijing (CN)

(21) Appl. No.: 17/637,690

(22) PCT Filed: Aug. 21, 2020
(86) PCT No.: PCT/CN2020/110442

§ 371 Date: Feb. 23, 2022

(30) Foreign Application Priority Data
Aug. 23, 2019 (CN) 201910785651.9
Jan. 21, 2020 (CN) 202010072446.0
Aug. 20, 2020 (CN) 202010840485.0

(51) Int. Cl. C07F 9/6558; C07F 9/6561

(52) U.S. Cl. CPC C07F 9/65583; C07F 9/6561

(57) ABSTRACT

A new compound of general formula (X) inhibiting and inducing degradation of an EGFR and ALK, and a pharmaceutical composition containing said compound. The compound and the pharmaceutical composition can be used for treating diseases related to the EGFR and the ALK kinase, such as cancer. The present invention further provides the preparation and use of the compound.

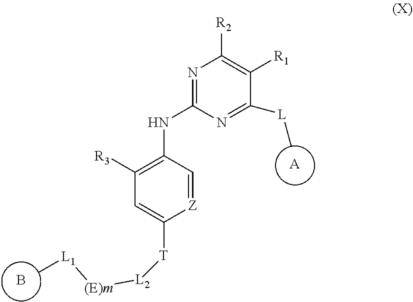

### CROSS REFERENCE TO RELATED APPLICATIONS

This application is a national phase entry of International Application No. PCT/CN2020/110442 filed on Aug. 21, 2020, which claims the priority of the Chinese Patent Application No. 201910785651.9 filed on Aug. 23, 2019, the Chinese Patent Application No. 202010072446.0 filed on Jan. 21, 2020, and the Chinese Patent Application No. 202010840485.0 filed on Aug. 20, 2020, which are incorporated herein by reference in their entireties for all purposes.

### FIELD OF THE INVENTION

The present disclosure relates to the field of medicine, specifically, the present disclosure provides compounds that can inhibit or induce the degradation of EGFR and ALK, and preparation and application thereof.

### BACKGROUND OF THE INVENTION

Lung cancer is one of the most common malignant tumors. In 2018, there were 2.1 million new lung cancer cases worldwide, accounting for 11.6% of all new tumor cases; there were 1.8 million deaths, accounting for 18.4% of all tumor deaths, wherein non-small cell lung cancer (NSCLC) accounts for 80%-85% of the total number of lung cancer. Epithelial growth factor receptor (EGFR) and anaplastic lymphoma kinase (ALK) are driving genes of common non-small cell lung cancer.

In non-small cell lung cancer, about 50% of Chinese patients and 11-16% of patients in western countries have EGFR gene mutations, wherein the most common mutation types are exon 19 deletion mutation (del E746-A750) and exon 21 L858R point mutation, accounting for about 90% of all EGFR mutation populations. EGFR small molecule inhibitors are the standard first-line treatment for non-small cell lung cancer with EGFR gene mutation and have been widely used in the field of lung cancer treatment. They competitively bind to EGFR with endogenous ligands, and inhibit the activation of tyrosine kinases, thereby blocking the EGFR signaling pathway, inhibiting tumor cell proliferation and metastasis, and promoting a series of biological effects such as tumor cell apoptosis.

The first-generation EGFR small-molecule inhibitors Gefitinib and Erlotinib have been used to treat advanced non-small cell lung cancer with activating EGFR mutations (L858R, del E746-A750). However, patients develop resistance after 10-12 months of administration of Gefitinib and Erlotinib, wherein more than 50% of drug-resistant patients are due to the secondary mutation of T790M in EGFR. Afatinib, the second-generation irreversible EGFR inhibitor, is effective for advanced non-small cell lung cancer patients with activating EGFR mutations (L858R, del E746-A750), but cannot resolve the clinical drug resistance caused by EGFR T790M mutation. Moreover, Afatinib lacks selectivity to wild-type EGFR and has great toxicity. Osimertinib, the third-generation irreversible inhibitor, overcomes the drug resistance caused by EGFR T790M and can effectively treat advanced non-small cell lung cancer patients with drug resistance caused by EGFR T790M mutation. Although Osimertinib has achieved great success in the clinical treatment of non-small cell lung cancer with EGFR T790M mutant, some patients who benefits from the treatment develops drug resistance after 9-14 months of treatment (Nature Medicine, 2015, 21(6), 560-562). Studies have shown that up to 22% of patients with drug resistance of Osimertinib are due to EGFR C797S mutation (JAMA Oncol. 2018; 4(11):1527-1534). EGFR C797S mutation causes cysteine at position 797 to be mutated into serine, and Osimertinib cannot covalently bind to EGFR, resulting in drug resistance. At present, there is no effective single EGFR inhibitor for EGFR C797S in clinical practice. Therefore, the development of a new generation of EGFR inhibitors to meet the needs of clinical treatment is an urgent problem to be solved.

Anaplastic lymphoma kinase (ALK) is a receptor tyrosine protein kinase. ALK gene rearrangement, point mutation and gene amplification can lead to carcinogenesis in the body. ALK rearrangement gene is a strong oncogenic driver gene, wherein echinoderm microtubule associated protein like 4 (EML4-ALK) and nucleophosmin (NPM-ALK) are common types. ALK gene rearrangement leads to the phosphorylation of ALK before dimer formation. Therefore, ALK fusion protein will continue to be activated and activate its downstream pathway, resulting in excessive cell proliferation and tumorigenesis. In non-small cell lung cancer, 3-7% of patients have ALK gene rearrangement. Small molecule inhibitors targeting ALK have been widely used in clinical practice. They competitively bind to ALK with endogenous ligands, and inhibit the activation of tyrosine kinases, thereby blocking the ALK signaling pathway, and inhibiting a series of biological effects such as tumor cell proliferation and metastasis. At present, the ALK inhibitors on the market include crizotinib, ceritinib, alectinib, brigatinib and loratinib, but it is inevitable that these inhibitors have drug resistance. Common ALK mutations leading to resistance are L1196M, G1269A, S1206Y, G1202R, C1156Y, L1198F and so on. Therefore, it is of great significance to develop new ALK inhibitors to meet the needs of clinical treatment.

Ubiquitin-proteasome system (UPS) is a multi-component system of intracellular protein degradation, which is involved in important physiological and biochemical processes such as cell growth and differentiation, DNA replication and repair, cell metabolism, immune response and so on. Protein degradation mediated by the ubiquitin-proteasome pathway is an important mechanism of the body for regulating intracellular protein level and function, and plays an important role in maintaining protein homeostasis in vivo. Inducing the degradation of EGFR or ALK through the intracellular ubiquitin-proteasome pathway provides a new idea for the treatment of cancer.

The present disclosure provides compounds that can inhibit and/or induce the degradation of EGFR and ALK, and preparation and application thereof.

### SUMMARY OF THE INVENTION

In one aspect, the present disclosure provides a compound of general formula (X), or a pharmaceutically acceptable salt, enantiomer, diastereomer, racemate, solvate, hydrate, polymorph, prodrug, or isotopic variant thereof, or mixture thereof, which can be used for treating diseases mediated by EGFR and/or ALK kinase, such as cancer.

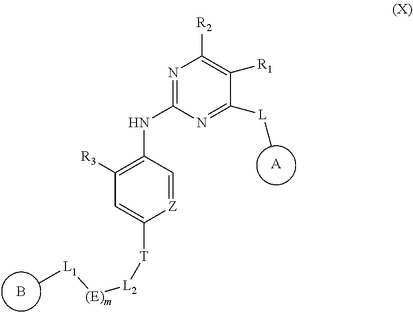

wherein

ring A is selected from the following optionally substituted groups: C₃₋₇ cycloalkyl, 4-to 8-membered heterocyclyl, C₆₋₁₀ aryl or 5- to 10-membered heteroaryl, wherein the substituent is selected from halogen, —CN, C₁₋₆ alkyl, C₁₋₆ haloalkyl, C₂₋₆ alkenyl, C₃₋₇ cycloalkyl, 4- to 8-membered heterocyclyl, —P(O)(C₁₋₆ alkyl)₂, —P(O)(C₂₋₆ alkenyl)₂, —O—C₁₋₆ alkyl, —O—C₁₋₆ haloalkyl, —O—C₂₋₆ alkenyl, —O—C₃₋₇ cycloalkyl, —O-4- to 8-membered heterocyclyl, —NH—C₁₋₆ alkyl, —NH—C₂₋₆ alkenyl, —NH—C₃₋₇ cycloalkyl, —NH-4- to 8-membered heterocyclyl, —C(O)—C₁₋₆ alkyl, —C(O)—C₂₋₆ alkenyl, —C(O)—C₃₋₇ cycloalkyl, —C(O)-4- to 8-membered heterocyclyl, —S(O)₂—C₁₋₆ alkyl, —S(O)₂—C₂₋₆ alkenyl, —S(O)₂—C₃₋₇ cycloalkyl, —S(O)₂-4- to 8-membered heterocyclyl, —NHC(O)—C₁₋₆ alkyl, —NHC(O)—C₂₋₆ alkenyl, —NHC(O)—C₃₋₇ cycloalkyl, —NHC(O)-4- to 8-membered heterocyclyl, —NHS(O)₂—C₁₋₆ alkyl, —NHS(O)₂—C₂₋₆ alkenyl, —NHS(O)₂—C₃₋₇ cycloalkyl or —NHS(O)₂-4- to 8-membered heterocyclyl;

ring B is selected from the following groups:

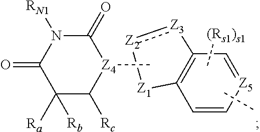

[symbol] represents single bond or double bond;

[symbol] represents that the point of attachment to the rest of the molecule can be located at the available point of the ring;

Z₁ is O, S, N or C atom, which is optionally substituted with one or two R_{Z1}; or Z₁ is absent, and thus Z₄ is connected to Z₂, Z₃ or the C atom on the aromatic ring connected to Z₁, and the Z₂ and the C atom on the aromatic ring that are connected to Z₁ are connected to R_{W} respectively; or Z₁, Z₂ and Z₃ are all absent, and thus Z₄ is connected to one of the C atoms on the aromatic ring connected to Z₁ or Z₃, and the other C atom on the aromatic ring is connected to R_{W};

Z₂ is O, S, N or C atom, which is optionally substituted with one or two R_{Z2};

Z₃ is O, S, N or C atom, which is optionally substituted with one or two R_{Z3}; with the proviso that when [symbol] represents double bond, Z₂ is N or C atom, and Z₃ is N or C atom;

Z₄ is N or CR_{Z4};

Z₅ is N or CR_{Z5};

R^{a}, R^{b} and R_{c} are independently H, halogen, —(CH₂)₀₋₅—OR″, —(CH₂)₀₋₅—NR″R″′, C₁₋₆ alkyl or C₁₋₆ haloalkyl; or R^{a} and R^{b} are taken together with the carbon atom to which they are attached to form C═O, C₃₋₇ cycloalkyl or 4- to 8-membered heterocyclyl; or R^{a} and R, are taken together with the carbon atoms to which they are attached to form C₃₋₇ cycloalkyl or 4- to 8-membered heterocyclyl; or R^{a} and R, are taken together to form bond;

R_{N1} is H, C₁₋₆ alkyl or C₁₋₆ haloalkyl, alternatively H;

R_{Z1} is absent, H, CN, halogen, —(CH₂)₀₋₅—OR″, —(CH₂)₀₋₅—NR″R″′, C₁₋₆ alkyl, C₁₋₆ haloalkyl, —(CH₂)₀₋₅—C₃₋₇ cycloalkyl or —(CH₂)₀₋₅-4- to 8-membered heterocyclyl; or two R_{Z1} are taken together with Z₁ to form C═O, C₃₋₇ cycloalkyl or 4- to 8-membered heterocyclyl;

R_{Z2} is absent, H, CN, halogen, —(CH₂)₀₋₅—OR″, —(CH₂)₀₋₅—NR″R″′, C₁₋₆ alkyl, C₁₋₆ haloalkyl, —(CH₂)₀₋₅—C₃₋₇ cycloalkyl or —(CH₂)₀₋₅-4- to 8-membered heterocyclyl; or two R_{Z2} are taken together with Z₂ to form C═O, C₃₋₇ cycloalkyl or 4- to 8-membered heterocyclyl;

R_{Z3} is absent, H, CN, halogen, —(CH₂)₀₋₅—OR″, —(CH₂)₀₋₅—NR″R″′, C₁₋₆ alkyl, C₁₋₆ haloalkyl, —(CH₂)₀₋₅—C₃₋₇ cycloalkyl or —(CH₂)₀₋₅-4- to 8-membered heterocyclyl; or two R_{Z3} are taken together with Z₃ to form C═O, C₃₋₇ cycloalkyl or 4- to 8-membered heterocyclyl;

R_{Z4} is H, CN, halogen, —(CH₂)₀₋₅—OR″, —(CH₂)₀₋₅—NR″R″′, C₁₋₆ alkyl or C₁₋₆ haloalkyl;

R_{Z5} is H, CN, halogen, —(CH₂)₀₋₅—OR″, —(CH₂)₀₋₅—NR″R″′, C₁₋₆ alkyl, C₁₋₆ haloalkyl, —(CH₂)₀₋₅—C₃₋₇ cycloalkyl or —(CH₂)₀₋₅-4- to 8-membered heterocyclyl;

or the ring where Z₄ is located is absent;

wherein R_{W} is H, CN, halogen, —(CH₂)₀₋₅—OR″, —(CH₂)₀₋₅—NR″R″′, —C₁₋₆ alkyl, C₁₋₆ haloalkyl, —(CH₂)₀₋₅—C₃₋₇ cycloalkyl, —(CH₂)₀₋₅-4- to 8-membered heterocyclyl, C₂₋₆ alkenyl, C₂-6 alkynyl, —(CH₂)₀₋₅—C₃₋₁₀ halocycloalkyl, —(CH₂)₀₋₅—C₆₋₁₀ aryl or —(CH₂)₀₋₅-5- to 14-membered heteroaryl;

R″ is H, C₁₋₆ alkyl, C₁₋₆ haloalkyl or —(CH₂)₀₋₅—C₃₋₇ cycloalkyl;

R″′ is H, C₁₋₆ alkyl or C₁₋₆ haloalkyl;

L₁ is selected from bond, —O—, —S(O)ₚ—, —S(O)(═NR*)—, —NR—, —CR^{#}R^{#}′—, —CₐR^{#}R^{#}′—C_{b}R^{#}R^{#}′—, —N═S(O)(R*)— or —S(O)(R*)═N—;

L₂ is selected from bond, —O—, —S(O)ₚ—, —S(O)(═NR*)—, —NR—, —CR^{#}R^{#}′—, —CₐR^{#}R^{#}′—C_{b}R^{#}R^{#}′—, —N═S(O)(R*)— or —S(O)(R*)═N—;

wherein one of CₐR^{#}R^{#}′ and C_{b}R^{#}R^{#}′ can be replaced by O, S(O)ₚ, S(O)(═NR*) or NR^{#}, and when one of CₐR^{#}R^{#}′ and C_{b}R^{#}R^{#}″ is replaced by O, S or NR^{#}, the other of CₐR^{#}R^{#}′ and C_{b}R^{#}R^{#}′ can also be replaced by S(O)_{q};

E is independently selected from: bond, —C_{c}R^{#}R^{#}′—CₐR^{#}R^{#}′—CₑR^{#}R^{#}′,

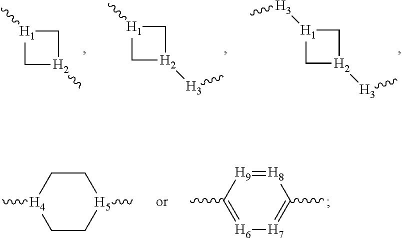

wherein one of C_{c}R^{#}R^{#}′, CₐR^{#}R^{#}′ or CₑR^{#}R^{#}′, or both of C_{c}R^{#}R^{#}′ and CₑR^{#}R^{#}′ can be replaced by O, S(O)ₚ, S(O)(═NR*) or NR^{#}, and when one of C_{c}R^{#}R^{#}′, CₐR^{#}R^{#}′ or CₑR^{#}R^{#}′ is replaced by O, S or NR^{#}, the other adjacent one or two of C_{c}R^{#}R^{#}′, C_{d}R^{#}R^{#}′ or CₑR^{#}R^{#}′ can also be replaced by S(O)_{q};

or two E moieties can be taken together to form —CH₂CH₂OCH₂CH₂—, —OCH₂CH₂CH₂CH₂—, —CH₂CH₂CH₂CH₂O—,

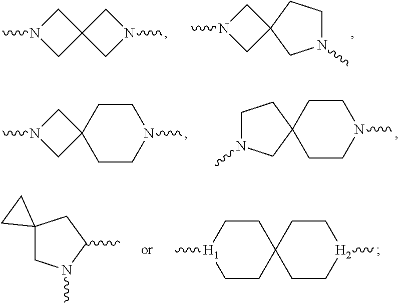

wherein [symbol] represents the point of attachment to L₁ or L₂;

[symbol]

H₁ and H₂ are N or C atom, H₃ is O, S, N or C atom, and H₁ and H₃, and H₂ and H₃ are not heteroatoms at the same time;

H₄ and H₅ are N or C atom;

H₆, H₇, H₈ and H₉ are C or N atom;

p is 0, 1 or 2;

q is 1 or 2;

R* is H, C₁₋₆ alkyl, C₁₋₆ haloalkyl, C₂₋₆ alkenyl, C₂₋₆ alkynyl, C₃₋₁₀ cycloalkyl, C₃₋₁₀ halocycloalkyl, 3- to 10-membered heterocyclyl, C₆₋₁₀ aryl or 5- to 14-membered heteroaryl;

R^{#} is H, halogen, C₁₋₆ alkyl, C₁₋₆ haloalkyl, C₂₋₆ alkenyl, C₂₋₆ alkynyl, C₃₋₁₀ cycloalkyl, C₃₋₁₀ halocycloalkyl, 3- to 10-membered heterocyclyl, C₆₋₁₀ aryl or 5- to 14-membered heteroaryl;

R^{#}′ is H, halogen, C₁₋₆ alkyl, C₁₋₆ haloalkyl, C₂₋₆ alkenyl, C₂₋₆ alkynyl, C₃₋₁₀ cycloalkyl, C₃₋₁₀ halocycloalkyl, 3- to 10-membered heterocyclyl, C₆₋₁₀ aryl or 5- to 14-membered heteroaryl;

or, R^{#} and R^{#} on adjacent atoms can be taken together to form bond, and R^{#}′ and R^{#}′ on adjacent atoms can be taken together to form bond;

or, R^{#} and R^{#}′ on the same or different atoms can be taken together to form ═O, or C₃₋₇ cycloalkyl, 4- to 8-membered heterocyclyl, C₆₋₁₀ aryl or 5- to 6-membered heteroaryl, wherein the C₃₋₇ cycloalkyl, 4- to 8-membered heterocyclyl, C₆₋₁₀ aryl or 5- to 6-membered heteroaryl is optionally substituted with Rₓ, and the Rₓ is H, CN, halogen, C₁₋₆ alkyl or C₁₋₆ haloalkyl;

m is 0, 1, 2, 3, 4, 5, 6, 7, 8, 9 or 10;

Rₛ₁ is selected from H, CN, halogen, —(CH₂)₀₋₅—OR″, —(CH₂)₀₋₅—NR″R″′, C₁₋₆ alkyl, C₁₋₆ haloalkyl, —(CH₂)₀₋₅-4- to 8-membered heterocyclyl, C₂₋₆ alkenyl, C₂₋₆ alkynyl, —(CH₂)₀₋₅—C₃₋₇ cycloalkyl, —(CH₂)₀₋₅—C₃₋₁₀ halocycloalkyl, —(CH₂)₀₋₅—C₆₋₁₀ aryl, —(CH₂)₀₋₅-5- to 14-membered heteroaryl, —C(O)R_{W}, —S(O)R_{W} or —S(O)₂R_{W};

s1 is 0, 1, 2 or 3;

R′ is selected from H, —C(O)—C₁₋₆ alkyl, —C(O)—C₁₋₆ haloalkyl, —C(O)—C₂₋₆ alkenyl or —C(O)—C₆₋₁₀ aryl;

L is bond, —O— or —NR—;

wherein R is H or C₁₋₆ alkyl;

Z is —N═ or —C(R^{#})═;

T is selected from bond, C₂₋₆ heteroalkylene, 4- to 12-membered heterocyclylene, or 5- to 6-membered heterocyclylene, wherein the 5- to 6-membered heterocyclylene is substituted with 5- to 6-membered heterocyclylene;

R₁ is selected from H, halogen, cyano, C₁₋₆ alkyl, C₃₋₇ cycloalkyl, 4- to 8-membered heterocyclyl, C₁₋₆ haloalkyl, C₂₋₆ alkenyl or C₂₋₆ alkynyl;

or, when L is —NR—, R₁ and R are taken together with the atoms to which they are attached to form optionally substituted 5- to 6-membered heterocyclyl, wherein the substituent is selected from halogen, oxo, C₁₋₆ alkyl, C₁₋₆ haloalkyl, or C₆₋₁₀ aryl, wherein the C₆₋₁₀ aryl is mono- or poly-substituted with halogen;

R₂ is H, halogen, hydroxyl, amino, C₁₋₆ alkyl or C₁₋₆ haloalkyl;

or R₁ and R₂ are taken together with the atoms to which they are attached to form 5- to 6-membered heterocyclyl or 5- to 6-membered heteroaryl;

R₃ is selected from H, —O—C₁₋₆ alkyl or —O—C₁₋₆ haloalkyl;

R₄ is selected from H, halogen, C₁₋₆ alkyl, C₁₋₆ haloalkyl, —NHC(O)—C₁₋₆ alkyl or —NHC(O)—C₂₋₆ alkenyl;

if the above groups are H or H-containing groups, the one or more H atom(s) may be substituted with D atom(s);

the groups containing OH, NH, NH₂, CH, CH₂, or CH₃ in L₁, E, L₂, and T, or the above alkyl, alkylene, haloalkyl, alkenyl, alkynyl, cycloalkyl, halocycloalkyl, heterocyclyl, aryl, and heteroaryl are each optionally substituted with 1, 2, 3 or more R^{s} at each occurrence, and the R^{s} is independently selected from the following groups at each occurrence: halogen, hydroxyl, amino, cyano, nitro, C₁₋₆ alkyl, C₁₋₆ haloalkyl, C₂₋₆ alkenyl, C₂₋₆ alkynyl, C₃₋₁₀ cycloalkyl, C₃. 10 halocycloalkyl, 3- to 10-membered heterocyclyl, C₆₋₁₀ aryl, 5- to 14-membered heteroaryl, C₆₋₁₂ aralkyl, —OR^{a′}, —OC(O)R^{a′}, —C(O)R^{a′}, —C(O)OR^{a′}, —C(O)NR^{a′}R^{b′}, —S(O)R^{a′}, —S(O)ₙOR^{a′}, —S(O)ₙNR^{a′}R^{b′}, —NR^{a′}R^{b′}, —NR^{a′}C(O)R^{b′}, —NR^{a′}—C(O)OR^{b′}, —NR^{a′}—S(O)ₙ—R^{b′}, —NR^{a′}C(O)NR^{a′}R^{b′}, —C₁₋₆ alkylene-R^{a′}, —C₁₋₆ alkylene-OR^{a′}, —C₁₋₆ alkylene-OC(O)R^{a′}, —C₁₋₆ alkylene-C(O)OR^{a′}, —C₁₋₆ alkylene-S(O)ₙR^{a′}, —C₁₋₆ alkylene-S(O)ₙOR^{a′}, —C₁₋₆ alkylene-OC(O)NR^{a′}, —C₁₋₆ alkylene-C(O)NR^{a′}R′, —C₁₋₆ alkylene-NR^{a′}—C(O)NR^{a′}R′, —C₁₋₆ alkylene-OS(O)ₙR^{a′}, —C₁₋₆ alkylene-S(O)ₙNR^{a′}R′, —C₁₋₆ alkylene-NR^{a′}—S(O)ₙNR^{a′}R′, —C₁₋₆ alkylene-NR^{a′}R′ and —O—C₁₋₆ alkylene-NR^{a′}R′, and wherein the hydroxyl, amino, alkyl, alkylene, cycloalkyl, heterocyclyl, aryl, heteroaryl and aralkyl described with respect to the substituent R^{s} are further optionally substituted with 1, 2, 3 or more substituents independently selected from: halogen, OH, amino, cyano, nitro, C₁₋₆ alkyl, C₁₋₆ haloalkyl, C₁₋₆ alkyl hydroxyl, C₃₋₆ cycloalkyl, 3- to 10-membered heterocyclyl, C₆₋₁₀ aryl, 5- to 14-membered heteroaryl and C₆₋₁₂ aralkyl;

n is independently 1 or 2 at each occurrence;

R^{a′} and R^{b′} are each independently selected from H, C₁₋₆ alkyl, C₂₋₆ alkenyl, C₂₋₆ alkynyl, C₁₋₆ alkyl-O—, C₁₋₆ alkyl-S—, C₃₋₁₀ cycloalkyl, 3- to 10-membered heterocyclyl, C₆₋₁₀ aryl, 5- to 14-membered heteroaryl and C₆₋₁₂ aralkyl at each occurrence.

In another aspect, the present disclosure provides a pharmaceutical composition comprising a compound disclosed herein, and optionally pharmaceutically acceptable excipients.

In another aspect, the present disclosure provides a pharmaceutical composition comprising a compound disclosed herein and pharmaceutically acceptable excipients, which further comprises other therapeutic agent(s).

In another aspect, the present disclosure provides a kit comprising a compound disclosed herein, other therapeutic agent(s), and pharmaceutically acceptable carriers, adjuvants or vehicles.

In another aspect, the present disclosure provides a use of a compound disclosed herein in the manufacture of a medicament for treating and/or preventing a disease mediated by EGFR and/or ALK kinase.

In another aspect, the present disclosure provides a method of treating and/or preventing a disease mediated by EGFR and/or ALK kinase in a subject, comprising administering to the subject a compound disclosed herein or a composition disclosed herein.

In another aspect, the present disclosure provides a compound disclosed herein or a composition disclosed herein, for use in treating and/or preventing a disease mediated by EGFR and/or ALK kinase.

In a specific embodiment, the diseases treated by the present disclosure include cancer, such as ovarian cancer, cervical cancer, colorectal cancer, breast cancer, pancreatic cancer, glioma, glioblastoma, melanoma, prostate cancer, leukemia, lymphoma, non-Hodgkin's lymphoma, gastric cancer, lung cancer, hepatocellular cancer, stomach cancer, gastrointestinal stromal tumor (GIST), thyroid cancer, cancer of bile duct, endometrial cancer, kidney cancer, anaplastic large cell lymphoma, acute myeloid leukemia (AML), multiple myeloma, melanoma, and mesothelioma.

Other objects and advantages of the present disclosure will be apparent to those skilled in the art from the following specific embodiments, examples and claims disclosed herein.

### Definition

### Chemical Definitions

Definitions of specific functional groups and chemical terms are described in more detail below.

When a range of values is listed, it is intended to encompass each value and sub-range within the range. For example, “C₁₋₆ alkyl” is intended to include C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅ and C₅₋₆ alkyl.

“C₁₋₆ alkyl” refers to a radical of a straight or branched, saturated hydrocarbon group having 1 to 6 carbon atoms. In some embodiments, C₁₋₄ alkyl is alternative. Examples of C₁₋₆ alkyl include methyl (C₁), ethyl (C₂), n-propyl (C₃), iso-propyl (C₃), n-butyl (C₄), tert-butyl (C₄), sec-butyl (C₄), iso-butyl (C₄), n-pentyl (C₅), 3-pentyl (C₅), pentyl (C₅), neopentyl (C₅), 3-methyl-2-butyl (C₅), tert-pentyl (C₅) and n-hexyl (C₆). The term “C₁₋₆ alkyl” also includes heteroalkyl, wherein one or more (e.g., 1, 2, 3 or 4) carbon atoms are substituted with heteroatoms (e.g., oxygen, sulfur, nitrogen, boron, silicon, phosphorus). Alkyl groups can be optionally substituted with one or more substituents, for example, with 1 to 5 substituents, 1 to 3 substituents or 1 substituent. Conventional abbreviations of alkyl include Me (—CH₃), Et (—CH₂CH₃), iPr (—CH(CH₃)₂), nPr (—CH₂CH₂CH₃), n-Bu (—CH₂CH₂CH₂CH₃) or i-Bu (—CH₂CH(CH₃)₂).

“C₂₋₆ alkenyl” refers to a radical of a straight or branched hydrocarbon group having 2 to 6 carbon atoms and at least one carbon-carbon double bond. In some embodiments, C₂₋₄ alkenyl is alternative. Examples of C₂₋₆ alkenyl include vinyl (C₂), 1-propenyl (C₃), 2-propenyl (C₃), 1-butenyl (C₄), 2-butenyl (C₄), butadienyl (C₄), pentenyl (C₅), pentadienyl (C₅), hexenyl (C₆), etc. The term “C₂₋₆ alkenyl” also includes heteroalkenyl, wherein one or more (e.g., 1, 2, 3 or 4) carbon atoms are replaced by heteroatoms (e.g., oxygen, sulfur, nitrogen, boron, silicon, phosphorus). The alkenyl groups can be optionally substituted with one or more substituents, for example, with 1 to 5 substituents, 1 to 3 substituents or 1 substituent.

“C₂₋₆ alkynyl” refers to a radical of a straight or branched hydrocarbon group having 2 to 6 carbon atoms, at least one carbon-carbon triple bond and optionally one or more carbon-carbon double bonds. In some embodiments, C₂₋₄ alkynyl is alternative. Examples of C₂₋₆ alkynyl include, but are not limited to, ethynyl (C₂), 1-propynyl (C₃), 2-propynyl (C₃), 1-butynyl (C₄), 2-butynyl (C₄), pentynyl (C₅), hexynyl (C₆), etc. The term “C₂₋₆ alkynyl” also includes heteroalkynyl, wherein one or more (e.g., 1, 2, 3 or 4) carbon atoms are replaced by heteroatoms (e.g., oxygen, sulfur, nitrogen, boron, silicon, phosphorus). The alkynyl groups can be substituted with one or more substituents, for example, with 1 to 5 substituents, 1 to 3 substituents or 1 substituent.

“C₁₋₆ alkylene, C₂₋₆ alkenylene or C₂₋₆ alkynylene” refers to a divalent group of the “C₁₋₆ alkyl, C₂₋₆ alkenyl or C₂₋₆ alkynyl” as defined above.

“C₁₋₆ alkylene” refers to a divalent group formed by removing another hydrogen of the C₁₋₆ alkyl, and can be substituted or unsubstituted. In some embodiments, C₁₋₄ alkylene is yet alternative. The unsubstituted alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), etc. Examples of substituted alkylene groups, such as those substituted with one or more alkyl (methyl) groups, include, but are not limited to, substituted methylene (—CH(CH₃)—, —C(CH₃)₂—), substituted ethylene (—CH(CH₃)CH₂—, —CH₂CH(CH₃)—, —C(CH₃)₂CH₂—, —CH₂C(CH₃)₂—), substituted propylene (—CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—), etc.

“C₂₋₆ alkenylene” refers to a C₂₋₆ alkenyl group wherein another hydrogen is removed to provide a divalent radical of alkenylene, and which may be substituted or unsubstituted. In some embodiments, C₂₋₄ alkenylene is yet alternative. Exemplary unsubstituted alkenylene groups include, but are not limited to, ethenylene (—CH═CH—) and propenylene (e.g., —CH═CHCH₂—, —CH₂—CH═CH—). Exemplary substituted alkenylene groups, e.g., substituted with one or more alkyl (methyl) groups, include but are not limited to, substituted ethylene (—C(CH₃)═CH—, —CH═C(CH₃)—), substituted propylene (e.g., —C(CH₃)═CHCH₂—, —CH═C(CH₃)CH₂—, —CH═CHCH(CH₃)—, —CH═CHC(CH₃)₂—, —CH(CH₃)—CH═CH—, —C(CH₃)₂—CH═CH—, —CH₂—C(CH₃)═CH—, —CH₂—CH═C(CH₃)—), and the like.

“C₂₋₆ alkynylene” refers to a C₂₋₆ alkynyl group wherein another hydrogen is removed to provide a divalent radical of alkynylene, and which may be substituted or unsubstituted. In some embodiments, C₂₋₄ alkynylene is yet alternative. Exemplary alkynylene groups include, but are not limited to, ethynylene (—C≡C—), substituted or unsubstituted propynylene (—C≡CCH₂—), and the like.

“C₁₋₆ heteroalkyl” refers to C₁₋₆ alkyl, as defined herein, which further contains one or more (e.g., 1, 2, 3 or 4) heteroatoms (e.g., oxygen, sulfur, nitrogen, boron, silicon, phosphorus) in the parent carbon chain, wherein one or more heteroatoms are between adjacent carbon atoms in the parent carbon chain, and/or one or more heteroatoms are between the carbon atom and the parent molecule, that is, between the connection points. The point of attachment between C₁₋₆ heteroalkyl and the parent molecule can be a carbon atom or a heteroatom.

“C₂₋₆ heteroalkylene” refers to a divalent group formed by removing another hydrogen of C₁₋₆ heteroalkyl, and can be substituted or unsubstituted. The point of attachment of C₁₋₆ heteroalkylene to other parts of the parent molecule can be two carbon atoms, or two heteroatoms, or one carbon atom and one heteroatom.

“Halo” or “halogen” refers to fluorine (F), chlorine (Cl), bromine (Br) and iodine (I).

Thus, “C₁₋₆ haloalkyl” refers to the above “C₁₋₆ alkyl”, which is substituted by one or more halogen. In some embodiments, C₁₋₄ haloalkyl is yet alternative, and still alternatively C₁. 2 haloalkyl. Exemplary haloalkyl groups include, but are not limited to, —CF₃, —CH₂F, —CHF₂, —CHFCH₂F, —CH₂CHF₂, —CF₂CF₃, —CCl₃, —CH₂Cl, —CHCl₂, 2,2,2-trifluoro-1,1-dimethyl-ethyl, and the like. The haloalkyl can be substituted at any available point of attachment, for example, with 1 to 5 substituents, 1 to 3 substituents or 1 substituent.

“C₃₋₁₀ cycloalkyl” refers to a radical of a non-aromatic cyclic hydrocarbon group having from 3 to 10 ring carbon atoms and zero heteroatoms. In some embodiments, C₃₋₇ cycloalkyl and C₃₋₆ cycloalkyl are yet alternative, and still alternatively C₅₋₆ cycloalkyl. The cycloalkyl also includes a ring system in which the cycloalkyl described herein is fused with one or more aryl or heteroaryl groups, wherein the point of attachment is on the cycloalkyl ring, and in such case, the number of carbon atoms continues to represent the number of carbon atoms in the cycloalkyl system. Exemplary cycloalkyl groups include, but are not limited to, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), etc. The cycloalkyl can be substituted with one or more substituents, for example, with 1 to 5 substituents, 1 to 3 substituents or 1 substituent.

“C₃₋₁₀ halocycloalkyl” refers to the above “C₃₋₁₀ cycloalkyl”, which is substituted by one or more halogen.

“3- to 12-membered heterocyclyl” refers to a radical of 3- to 12-membered non-aromatic ring system having ring carbon atoms and 1 to 5 ring heteroatoms, wherein each of the heteroatoms is independently selected from nitrogen, oxygen, sulfur, boron, phosphorus and silicon. In the heterocyclyl containing one or more nitrogen atoms, the point of attachment can be a carbon or nitrogen atom as long as the valence permits. In some embodiments, 4- to 12-membered heterocyclyl is alternative, which is a radical of 4- to 12-membered non-aromatic ring system having ring carbon atoms and 1 to 5 ring heteroatoms. In some embodiments, 3- to 10-membered heterocyclyl is alternative, which is a radical of 3- to 10-membered non-aromatic ring system having ring carbon atoms and 1 to 5 ring heteroatoms. In some embodiments, 3- to 8-membered heterocyclyl is alternative, which is a radical of 3- to 8-membered non-aromatic ring system having ring carbon atoms and 1 to 4 ring heteroatoms. 3- to 6-membered heterocyclyl is alternative, which is a radical of 3- to 6-membered non-aromatic ring system having ring carbon atoms and 1 to 3 ring heteroatoms. 4- to 8-membered heterocyclyl is alternative, which is a radical of 4- to 8-membered non-aromatic ring system having ring carbon atoms and 1 to 3 ring heteroatoms. 5- to 6-membered heterocyclyl is more alternative, which is a radical of 5- to 6-membered non-aromatic ring system having ring carbon atoms and 1 to 3 ring heteroatoms. The heterocyclyl also includes a ring system wherein the heterocyclyl described above is fused with one or more cycloalkyl groups, wherein the point of attachment is on the cycloalkyl ring, or the heterocyclyl described above is fused with one or more aryl or heteroaryl groups, wherein the point of attachment is on the heterocyclyl ring; and in such cases, the number of ring members continues to represent the number of ring members in the heterocyclyl ring system. Exemplary 3-membered heterocyclyl groups containing one heteroatom include, but are not limited to, aziridinyl, oxiranyl and thiorenyl. Exemplary 4-membered heterocyclyl groups containing one heteroatom include, but are not limited to, azetidinyl, oxetanyl and thietanyl. Exemplary 5-membered heterocyclyl groups containing one heteroatom include, but are not limited to, tetrahydrofuranyl, dihydrofuranyl, tetrahydrothiophenyl, dihydrothienyl, pyrrolidinyl, dihydropyrrolyl and pyrrolyl-2,5-dione. Exemplary 5-membered heterocyclyl groups containing two heteroatoms include, but are not limited to, dioxolanyl, oxasulfuranyl, disulfuranyl, and oxazolidin-2-one. Exemplary 5-membered heterocyclyl groups containing three heteroatoms include, but are not limited to, triazolinyl, oxadiazolinyl, and thiadiazolinyl. Exemplary 6-membered heterocyclyl groups containing one heteroatom include, but are not limited to, piperidyl, tetrahydropyranyl, dihydropyridyl and thianyl. Exemplary 6-membered heterocyclyl groups containing two heteroatoms include, but are not limited to, piperazinyl, morpholinyl, dithianyl and dioxanyl. Exemplary 6-membered heterocyclyl groups containing three heteroatoms include, but are not limited to, triazinanyl. Exemplary 7-membered heterocycyl groups containing one heteroatom include, but are not limited to, azepanyl, oxepanyl and thiepanyl. Exemplary 5-membered heterocyclyl groups fused with a C₆ aryl (also referred as 5,6-bicyclic heterocyclyl herein) include, but are not limited to, indolinyl, isoindolinyl, dihydrobenzofuranyl, dihydrobenzothiophenyl, benzoxazolinonyl, etc. Exemplary 6-membered heterocyclyl groups fused with a C₆ aryl (also referred as 6,6-bicyclic heterocyclyl herein) include, but are not limited to, tetrahydroquinolinyl, tetrahydroisoquinolinyl, etc. The heterocyclyl can be substituted with one or more substituents, for example, with 1 to 5 substituents, 1 to 3 substituents or 1 substituent.

“4- to 12-membered heterocyclylene” and “5- to 6-membered heterocyclylene” refer to the above “4- to 12-membered heterocyclyl” and “5- to 6-membered heterocyclyl”, respectively, wherein another hydrogen is removed and formed divalent groups, and can be substituted or unsubstituted.

“C₆₋₁₀ aryl” refers to a radical of monocyclic or polycyclic (e.g., bicyclic) 4n+2 aromatic ring system having 6-10 ring carbon atoms and zero heteroatoms (e.g., having 6 or 10 shared π electrons in a cyclic array). In some embodiments, the aryl group has six ring carbon atoms (“C₆ aryl”; for example, phenyl). In some embodiments, the aryl group has ten ring carbon atoms (“C₁₀ aryl”; for example, naphthyl, e.g., 1-naphthyl and 2-naphthyl). The aryl group also includes a ring system in which the aryl ring described above is fused with one or more cycloalkyl or heterocyclyl groups, and the point of attachment is on the aryl ring, in which case the number of carbon atoms continues to represent the number of carbon atoms in the aryl ring system. The aryl can be substituted with one or more substituents, for example, with 1 to 5 substituents, 1 to 3 substituents or 1 substituent.

“C₆₋₁₂ aralkyl” refers to the group —R—R′, wherein R is alkyl, R′ is aryl, and alkyl and aryl have a total of 6-12 carbon atoms.

“5- to 14-membered heteroaryl” refers to a radical of 5- to 14-membered monocyclic or bicyclic 4n+2 aromatic ring system (e.g., having 6, 10 or 14 shared R electrons in a cyclic array) having ring carbon atoms and 1-4 ring heteroatoms, wherein each heteroatom is independently selected from nitrogen, oxygen and sulfur. In the heteroaryl group containing one or more nitrogen atoms, the point of attachment can be a carbon or nitrogen atom as long as the valence permits. Heteroaryl bicyclic systems may include one or more heteroatoms in one or two rings. Heteroaryl also includes ring systems wherein the heteroaryl ring described above is fused with one or more cycloalkyl or heterocyclyl groups, and the point of attachment is on the heteroaryl ring. In such case, the number the carbon atoms continues to represent the number of carbon atoms in the heteroaryl ring system. In some embodiments, 5- to 10-membered heteroaryl groups are alternative, which are radicals of 5- to 10-membered monocyclic or bicyclic 4n+2 aromatic ring systems having ring carbon atoms and 1-4 ring heteroatoms. In other embodiments, 5- to 6-membered heteroaryl groups are yet alternative, which are radicals of 5- to 6-membered monocyclic or bicyclic 4n+2 aromatic ring systems having ring carbon atoms and 1-4 ring heteroatoms. Exemplary 5-membered heteroaryl groups containing one heteroatom include, but are not limited to, pyrrolyl, furyl and thienyl. Exemplary 5-membered heteroaryl groups containing two heteroatoms include, but are not limited to, imidazolyl, pyrazolyl, oxazolyl, isoxazolyl, thiazolyl, and isothiazolyl. Exemplary 5-membered heteroaryl groups containing three heteroatoms include, but are not limited to, triazolyl, oxadiazolyl (such as, 1,2,4-oxadiazoly), and thiadiazolyl. Exemplary 5-membered heteroaryl groups containing four heteroatoms include, but are not limited to, tetrazolyl. Exemplary 6-membered heteroaryl groups containing one heteroatom include, but are not limited to, pyridyl. Exemplary 6-membered heteroaryl groups containing two heteroatoms include, but are not limited to, pyridazinyl, pyrimidinyl, and pyrazinyl. Exemplary 6-membered heteroaryl groups containing three or four heteroatoms include, but are not limited to, triazinyl and tetrazinyl, respectively. Exemplary 7-membered heteroaryl groups containing one heteroatom include, but are not limited to, azepinyl, oxepinyl, and thiepinyl. Exemplary 5,6-bicyclic heteroaryl groups include, but are not limited to, indolyl, isoindolyl, indazolyl, benzotriazolyl, benzothiophenyl, isobenzothiophenyl, benzofuranyl, benzoisofuranyl, benzimidazolyl, benzoxazolyl, benzoisoxazolyl, benzoxadiazolyl, benzothiazolyl, benzoisothiazolyl, benzothiadiazolyl, indolizinyl and purinyl. Exemplary 6,6-bicyclic heteroaryl groups include, but are not limited to, naphthyridinyl, pteridinyl, quinolyl, isoquinolyl, cinnolinyl, quinoxalinyl, phthalazinyl and quinazolinyl. The heteroaryl can be substituted with one or more substituents, for example, with 1 to 5 substituents, 1 to 3 substituents or 1 substituent.

“Oxo” represents ═O.

Alkyl, alkenyl, alkynyl, cycloalkyl, heterocyclyl, aryl and heteroaryl as defined herein are optionally substituted groups.

Exemplary substituents on carbon atoms include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃+X—, —N(OR^{cc})R^{bb}, —SH, —SR^{aa}, —SSR^{cc}, —C(═O)R^{aa}, —CO₂H, —CHO, —C(OR^{cc})₂, —CO₂R′, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{b′}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R″)₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂—OP(═O)(NR^{bb})₂, —NR^{bb}P(═O)(OR^{cc})₂—NR^{bb}P(═O)(NR^{b})₂—P(R_{c})₂, —P(R^{cc})₃, —OP(R^{cc})₂, —OP(R^{cc})₃, —B(R^{aa})₂, —B(OR^{cc})₂, —BR^{aa}(OR^{cc}), alkyl, haloalkyl, alkenyl, alkynyl, cycloalkyl, heterocyclyl, aryl and heteroaryl, wherein each of the alkyl, alkenyl, alkynyl, cycloalkyl, heterocyclyl, aryl and heteroaryl is independently substituted with 0, 1, 2, 3, 4 or 5 R^{dd} groups;

or two geminal hydrogen on a carbon atom are replaced with ═O, ═S, ═NN(R^{bb})₂, ═NNR^{bb}C(═O)R^{aa}, —NNR^{bb}C(═O)OR^{aa}, —NNR^{bb}S(═O)₂R^{aa}—NR^{bb} or ═NOR^{cc} groups;

each of the R^{aa} is independently selected from alkyl, haloalkyl, alkenyl, alkynyl, cycloalkyl, heterocyclyl, aryl and heteroaryl, or two of the R^{a} groups are combined to form a heterocyclyl or heteroaryl ring, wherein each of the alkyl, alkenyl, alkynyl, cycloalkyl, heterocyclyl, aryl and heteroaryl is independently substituted with 0, 1, 2, 3, 4 or 5 R^{dd} groups;

each of the R^{bb} is independently selected from hydrogen, —OH, —OR^{aa}, —N(R^{c})₂, —CN, —C(═O)R^{aa}, —C(═O)N(R_{c})₂, —CO₂R^{aa}, —SO₂R^{aa}, —C(═NR^{cc})OR^{aa}, —C(═NR^{cc})N(R^{cc})₂, —SO₂N(R_{c})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, alkyl, haloalkyl, alkenyl, alkynyl, cycloalkyl, heterocyclyl, aryl and heteroaryl, or two R^{bb} groups are combined to form a heterocyclyl or a heteroaryl ring, wherein each of the alkyl, alkenyl, alkynyl, cycloalkyl, heterocyclyl, aryl and heteroaryl is independently substituted with 0, 1, 2, 3, 4 or 5 R^{dd} groups;

each of the R^{cc} is independently selected from hydrogen, alkyl, haloalkyl, alkenyl, alkynyl, cycloalkyl, heterocyclyl, aryl and heteroaryl, or two R^{cc} groups are combined to form a heterocyclyl or a heteroaryl ring, wherein each of the alkyl, alkenyl, alkynyl, cycloalkyl, heterocyclyl, aryl and heteroaryl is independently substituted with 0, 1, 2, 3, 4 or 5 R^{dd} groups;

each of the R^{dd} is independently selected from halogen, —CN, —NO₂, —N₃, —SO₂H, —SO₃H, —OH, —OR^{ee}, —ON(R^{f})₂, —N(R^{f})₂, —N(R^{f})₃+X—, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R⁷)₂, —OC(═O)N(R^{f})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NRC(═NR^{ff})N(R^{ff})₂, —NR^{ff}SO₂R^{ee}, —SO₂N(R⁷)₂, —SO₂Re, —SO₂OR^{ee}, —OSO₂R^{ee}, —S(═O)R^{ee}, —Si(R^{ee})₃, —OSi(R^{ee})₃, —C(═S)N(R^{f})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, alkyl, haloalkyl, alkenyl, alkynyl, cycloalkyl, heterocyclyl, aryl, heteroaryl, wherein each of the alkyl, alkenyl, alkynyl, cycloalkyl, heterocyclyl, aryl and heteroaryl is independently substituted with 0, 1, 2, 3, 4 or 5 R^{gg} groups, or two geminal R^{dd} substituents can be combined to form ═O or ═S;

each of the R^{ee} is independently selected from alkyl, haloalkyl, alkenyl, alkynyl, cycloalkyl, aryl, heterocyclyl, and heteroaryl, wherein each of the alkyl, alkenyl, alkynyl, cycloalkyl, heterocyclyl, aryl and heteroaryl is independently substituted with 0, 1, 2, 3, 4 or 5 R^{gg} groups;

each of the R^{ff} is independently selected from hydrogen, alkyl, haloalkyl, alkenyl, alkynyl, cycloalkyl, heterocyclyl, aryl and heteroaryl, or two R^{f} groups are combined to form a heterocyclyl or a heteroaryl ring, wherein each of the alkyl, alkenyl, alkynyl, cycloalkyl, heterocyclyl, aryl and heteroaryl is independently substituted with 0, 1, 2, 3, 4 or 5 R⁹⁹ groups;

each of the R^{gg} is independently selected from halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃+X—, —NH(C₁₋₆ alkyl)₂+X—, —NH₂(C₁₋₆ alkyl)+X—, —NH₃+X—, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃, —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ haloalkyl, C₂-C₆ alkenyl, C₂-C₆ alkynyl, C₃-C₇ cycloalkyl, C₆-C₁₀ aryl, C₃-C₇ heterocyclyl, C₅-C₁₀ heteroaryl; or two geminal R⁹⁹ substituents may combine to form ═O or ═S; wherein X⁻ is a counter-ion.

Exemplary substituents on nitrogen atoms include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{a′}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SORa′, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, alkyl, haloalkyl, alkenyl, alkynyl, cycloalkyl, heterocyclyl, aryl and heteroaryl, or two R^{cc} groups attached to a nitrogen atom combine to form a heterocyclyl or a heteroaryl ring, wherein each of the alkyl, alkenyl, alkynyl, cycloalkyl, heterocyclyl, aryl and heteroaryl is independently substituted with 0, 1, 2, 3, 4 or 5 R^{dd} groups, and wherein R^{aa}, R^{bb}, R^{cc} and R^{dd} are as described herein.

### Other Definitions

The term “cancer” includes, but is not limited to, the following cancers: breast, ovary, cervix, prostate, testis, esophagus, stomach, skin, lung, bone, colon, pancreas, thyroid, biliary tract, buccal cavity and pharynx (mouth), lips, tongue, oral cavity, pharynx, small intestine, colorectal, large intestine, rectum, cancer of brain and central nervous system, glioblastoma, neuroblastoma, keratoacanthoma, epidermoid carcinoma, large cell carcinoma, adenocarcinoma, adenoma, follicular carcinoma, undifferentiated carcinoma, papillary carcinoma, seminoma, melanoma, sarcoma, bladder cancer, liver cancer, kidney cancer, bone marrow disorder, lymphatic disorder, Hodgkin's disease, hairy cell carcinoma and leukemia.

The term “treating” as used herein relates to reversing, alleviating or inhibiting the progression or prevention of the disorders or conditions to which the term applies, or of one or more symptoms of such disorders or conditions. The noun “treatment” as used herein relates to the action of treating, which is a verb, and the latter is as just defined.

The term “pharmaceutically acceptable salt” as used herein refers to those carboxylate and amino acid addition salts of the compounds of the present disclosure, which are suitable for the contact with patients' tissues within a reliable medical judgment, and do not produce inappropriate toxicity, irritation, allergy, etc. They are commensurate with a reasonable benefit/risk ratio, and are effective for their intended use. The term includes, if possible, the zwitterionic form of the compounds of the disclosure.

The pharmaceutically acceptable base addition salts are formed with metals or amines, such as alkali metal and alkaline earth metal hydroxides or organic amines. Examples of the metals used as cations include sodium, potassium, magnesium, calcium, etc. Examples of suitable amines are N,N′-dibenzylethylenediamine, chloroprocaine, choline, diethanolamine, ethylenediamine, N-methylglucamine and procaine.

The base addition salt of the acidic compound can be prepared by contacting the free acid form with a sufficient amount of the required base to form a salt in a conventional manner. The free acid can be regenerated by contacting the salt form with an acid in a conventional manner and then isolating the free acid. The free acid forms are somewhat different from their respective salt forms in their physical properties, such as solubility in polar solvents. But for the purposes of the present disclosure, the salts are still equivalent to their respective free acids.

The salts can be prepared from the inorganic acids, which include sulfates, pyrosulfates, bisulfates, sulfites, bisulfites, nitrates, phosphates, monohydrogen phosphates, dihydrogen phosphates, metaphosphates, pyrophosphates, chlorides, bromides and iodides. Examples of the acids include hydrochloric acid, nitric acid, sulfuric acid, hydrobromic acid, hydroiodic acid, phosphoric acid, etc. The representative salts include hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthalate, methanesulfonate, glucoheptanate, lactobionate, lauryl sulfonate, isethionate, etc. The salts can also be prepared from the organic acids, which include aliphatic monocarboxylic and dicarboxylic acids, phenyl-substituted alkanoic acids, hydroxyalkanoic acids, alkanedioic acid, aromatic acids, aliphatic and aromatic sulfonic acids, etc. The representative salts include acetate, propionate, octanoate, isobutyrate, oxalate, malonate, succinate, suberate, sebacate, fumarate, maleate, mandelate, benzoate, chlorobenzoate, methyl benzoate, dinitrobenzoate, naphthoate, besylate, tosylate, phenylacetate, citrate, lactate, maleate, tartrate, methanesulfonate, etc. The pharmaceutically acceptable salts can include cations based on alkali metals and alkaline earth metals, such as sodium, lithium, potassium, calcium, magnesium, etc., as well as non-toxic ammonium, quaternary ammonium, and amine cations including, but not limited to, ammonium, tetramethylammonium, tetraethylammonium, methylamine, dimethylamine, trimethylamine, triethylamine, ethylamine, etc. Salts of amino acids are also included, such as arginine salts, gluconates, galacturonates, etc. (for example, see Berge S. M. et al., “Pharmaceutical Salts,” J. Pharm. Sci., 1977; 66: 1-19 for reference).

“Subjects” to which administration is contemplated include, but are not limited to, humans (e.g., males or females of any age group, e.g., paediatric subjects (e.g., infants, children, adolescents) or adult subjects (e.g., young adults, middle-aged adults or older adults) and/or non-human animals, such as mammals, e.g., primates (e.g., cynomolgus monkeys, rhesus monkeys), cattle, pigs, horses, sheep, goats, rodents, cats and/or dogs. In some embodiments, the subject is a human. In some embodiments, the subject is a non-human animal. The terms “humam”, “patient” and “subject” can be used interchangeably herein.

“Disease,” “disorder,” and “condition” can be used interchangeably herein.

Unless indicated, otherwise the term “treatment” as used herein includes the effect on a subject who is suffering from a particular disease, disorder, or condition, which reduces the severity of the disease, disorder, or condition, or delays or slows the progression of the disease, disorder or condition (“therapeutic treatment”). The term also includes the effect that occurs before the subject begins to suffer from a specific disease, disorder or condition (“prophylactic treatment”).

Generally, the “effective amount” of a compound refers to an amount sufficient to elicit a target biological response. As understood by those skilled in the art, the effective amount of the compound of the disclosure can vary depending on the following factors, such as the desired biological endpoint, the pharmacokinetics of the compound, the diseases being treated, the mode of administration, and the age, health status and symptoms of the subjects. The effective amount includes therapeutically effective amount and prophylactically effective amount.

Unless indicated, otherwise the “therapeutically effective amount” of the compound as used herein is an amount sufficient to provide therapeutic benefits in the course of treating a disease, disorder or condition, or to delay or minimize one or more symptoms associated with the disease, disorder or condition. The therapeutically effective amount of a compound refers to the amount of the therapeutic agent that, when used alone or in combination with other therapies, provides a therapeutic benefit in the treatment of a disease, disorder or condition. The term “therapeutically effective amount” can include an amount that improves the overall treatment, reduces or avoids the symptoms or causes of the disease or condition, or enhances the therapeutic effect of other therapeutic agents.

Unless indicated, otherwise the “prophylactically effective amount” of the compound as used herein is an amount sufficient to prevent a disease, disorder or condition, or an amount sufficient to prevent one or more symptoms associated with a disease, disorder or condition, or an amount sufficient to prevent the recurrence of a disease, disorder or condition. The prophylactically effective amount of a compound refers to the amount of a therapeutic agent that, when used alone or in combination with other agents, provides a prophylactic benefit in the prevention of a disease, disorder or condition. The term “prophylactically effective amount” can include an amount that improves the overall prevention, or an amount that enhances the prophylactic effect of other preventive agents.

“Combination” and related terms refer to the simultaneous or sequential administration of the compounds of the present disclosure and other therapeutic agents. For example, the compounds of the present disclosure can be administered simultaneously or sequentially in separate unit dosage with other therapeutic agents, or simultaneously in a single unit dosage with other therapeutic agents.

### BRIEF DESCRIPTION OF THE DRAWINGS

FIG. 1 shows the effect of compound C176 on EGFR^{L858R/T790M/C797S} protein level.

FIG. 2 shows the effect of compound C213 on EGFR^{Dell9/T790M/C797S} protein level.

### DETAILED DESCRIPTION OF THE INVENTION

As used herein, the term “compound disclosed herein” refers to the following compounds of formula (X) (including sub general formulas, such as formula (I), (I-1), (I-5-1), etc), or a pharmaceutically acceptable salt, enantiomer, diastereomer, racemate, solvate, hydrate, polymorph, prodrug, or isotopic variant thereof, or mixture thereof.

In the present disclosure, compounds are named using standard nomenclature. For compounds having an asymmetric center, it should be understood, unless otherwise stated, that all optical isomers and mixtures thereof are included. Furthermore, unless otherwise specified, all isomer compounds and carbon-carbon double bonds included in the present disclosure may occur in the form of Z and E. Compounds which exist in different tautomeric forms, one of which is not limited to any particular tautomer, but is intended to cover all tautomeric forms.

In one embodiment, the present disclosure relates to a compound of general formula (X), or a pharmaceutically acceptable salt, enantiomer, diastereomer, racemate, solvate, hydrate, polymorph, prodrug, or isotopic variant thereof, or mixture thereof:

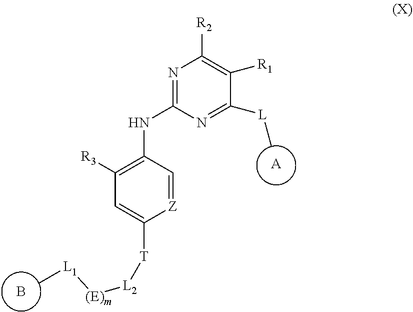

wherein

ring A is selected from the following optionally substituted groups: C₃₋₇ cycloalkyl, 4- to 8-membered heterocyclyl, C₆₋₁₀ aryl or 5- to 10-membered heteroaryl, wherein the substituent is selected from halogen, —CN, C₁₋₆ alkyl, C₁₋₆ haloalkyl, C₂₋₆ alkenyl, C₃₋₇ cycloalkyl, 4- to 8-membered heterocyclyl, —P(O)(C₁₋₆ alkyl)₂, —P(O)(C₂₋₆ alkenyl)₂, —O—C₁₋₆ alkyl, —O—C₁₋₆ haloalkyl, —O—C₂₋₆ alkenyl, —O—C₃₋₇ cycloalkyl, —O-4- to 8-membered heterocyclyl, —NH—C₁₋₆ alkyl, —NH—C₂₋₆ alkenyl, —NH—C₃₋₇ cycloalkyl, —NH-4- to 8-membered heterocyclyl, —C(O)—C₁₋₆ alkyl, —C(O)—C₂₋₆ alkenyl, —C(O)—C₃₋₇ cycloalkyl, —C(O)-4- to 8-membered heterocyclyl, —S(O)₂—C₁₋₆ alkyl, —S(O)₂—C₂₋₆ alkenyl, —S(O)₂—C₃₋₇ cycloalkyl, —S(O)₂-4- to 8-membered heterocyclyl, —NHC(O)—C₁₋₆ alkyl, —NHC(O)—C₂₋₆ alkenyl, —NHC(O)—C₃₋₇ cycloalkyl, —NHC(O)-4- to 8-membered heterocyclyl, —NHS(O)₂—C₁₋₆ alkyl, —NHS(O)₂—C₂₋₆ alkenyl, —NHS(O)₂—C₃₋₇ cycloalkyl or —NHS(O)₂-4- to 8-membered heterocyclyl;

ring B is selected from the following groups:

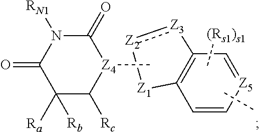

[symbol] represents single bond or double bond;

[symbol] represents that the point of attachment to the rest of the molecule can be located at the available point of the ring;

Z₁ is O, S, N or C atom, which is optionally substituted with one or two R_{Z1}; or Z₁ is absent, and thus Z₄ is connected to Z₂, Z₃ or the C atom connected to Z₁ on the aromatic ring, and the Z₂ and the C atom on the aromatic ring that are connected to Z₁ are connected to R_{W} respectively; or Z₁, Z₂ and Z₃ are all absent, and thus Z₄ is connected to one of the C atoms connected to Z₁ or Z₃ on the aromatic ring, and the other C atom on the aromatic ring is connected to R_{W};

Z₂ is O, S, N or C atom, which is optionally substituted with one or two R_{Z2};

Z₃ is O, S, N or C atom, which is optionally substituted with one or two R_{Z3}; with the proviso that when [symbol] represents double bond, Z₂ is N or C atom, and Z₃ is N or C atom;

Z₄ is N or CR_{Z4};

Z₅ is N or CR_{Z5};

R^{a}, R^{b} and R_{c} are independently H, halogen, —(CH₂)₀₋₅—OR″, —(CH₂)₀₋₅—NR″R″′, C₁₋₆ alkyl or C₁₋₆ haloalkyl; or R^{a} and R^{b} are taken together with the carbon atom to which they are attached to form C═O, C₃₋₇ cycloalkyl or 4- to 8-membered heterocyclyl; or, R^{a} and R, are taken together with the carbon atoms to which they are attached to form C₃₋₇ cycloalkyl or 4- to 8-membered heterocyclyl; or R^{a} and Re are taken together to form bond;

R_{N1} is H, C₁₋₆ alkyl or C₁₋₆ haloalkyl, alternatively H;

R_{Z1} is absent, H, CN, halogen, —(CH₂)₀₋₅—OR″, —(CH₂)₀₋₅—NR″R″′, C₁₋₆ alkyl, C₁₋₆ haloalkyl, —(CH₂)₀₋₅—C₃₋₇ cycloalkyl or —(CH₂)₀₋₅-4- to 8-membered heterocyclyl; or two R_{Z1} are taken together with Z₁ to form C═O, C₃₋₇ cycloalkyl or 4- to 8-membered heterocyclyl;

R_{Z2} is absent, H, CN, halogen, —(CH₂)₀₋₅—OR″, —(CH₂)₀₋₅—NR″R″′, C₁₋₆ alkyl, C₁₋₆ haloalkyl, —(CH₂)₀₋₅—C₃₋₇ cycloalkyl or —(CH₂)₀₋₅-4- to 8-membered heterocyclyl; or two R_{Z2} are taken together with Z₂ to form C═O, C₃₋₇ cycloalkyl or 4- to 8-membered heterocyclyl;

R_{Z3} is absent, H, CN, halogen, —(CH₂)₀₋₅—OR″, —(CH₂)₀₋₅—NR″R″′, C₁₋₆ alkyl, C₁₋₆ haloalkyl, —(CH₂)₀₋₅—C₃₋₇ cycloalkyl or —(CH₂)₀₋₅-4- to 8-membered heterocyclyl; or two R_{Z3} are taken together with Z₃ to form C═O, C₃₋₇ cycloalkyl or 4- to 8-membered heterocyclyl;

R_{Z4} is H, CN, halogen, —(CH₂)₀₋₅—OR″, —(CH₂)₀₋₅—NR″R″′, C₁₋₆ alkyl or C₁₋₆ haloalkyl;

R_{Z5} is H, CN, halogen, —(CH₂)₀₋₅—OR″, —(CH₂)₀₋₅—NR″R″′, C₁₋₆ alkyl, C₁₋₆ haloalkyl, —(CH₂)₀₋₅—C₃₋₇ cycloalkyl or —(CH₂)₀₋₅-4- to 8-membered heterocyclyl;

or the ring where Z₄ is located is absent;

wherein R_{W} is H, CN, halogen, —(CH₂)₀₋₅—OR″, —(CH₂)₀₋₅—NR″R″′, —C₁₋₆ alkyl, C₁₋₆ haloalkyl, —(CH₂)₀₋₅—C₃₋₇ cycloalkyl, —(CH₂)₀₋₅-4- to 8-membered heterocyclyl, C₂₋₆ alkenyl, C₂-6 alkynyl, —(CH₂)₀₋₅—C₃₋₁₀ halocycloalkyl, —(CH₂)₀₋₅—C₆₋₁₀ aryl or —(CH₂)₀₋₅-5- to 14-membered heteroaryl;

R″ is H, C₁₋₆ alkyl, C₁₋₆ haloalkyl or —(CH₂)₀₋₅—C₃₋₇ cycloalkyl;

R″′ is H, C₁₋₆ alkyl or C₁₋₆ haloalkyl;

L₁ is selected from bond, —O—, —S(O)ₚ—, —S(O)(═NR*)—, —NR—, —CR^{#}R^{#}′—, —CₐR^{#}R^{#}′—C_{b}R^{#}R^{#}′—, —N═S(O)(R*)— or —S(O)(R*)═N—;

L₂ is selected from bond, —O—, —S(O)ₚ—, —S(O)(═NR*)—, —NR—, —CR^{#}R^{#}′—, —CₐR^{#}R^{#}′—C_{b}R^{#}R^{#}′—, —N═S(O)(R*)— or —S(O)(R*)═N—;

wherein one of CₐR^{#}R^{#}′ and C_{b}R^{#}R^{#}′ can be replaced by O, S(O)ₚ, S(O)(═NR*) or NR^{#}, and when one of CₐR^{#}R^{#}′ and C_{b}R^{#}R^{#}″ is replaced by O, S or NR^{#}, the other of CₐR^{#}R^{#}′ and C_{b}R^{#}R^{#}′ can also be replaced by S(O)_{q};

E is independently selected from: bond, —C_{c}R^{#}R^{#}′—C_{d}R^{#}R^{#}′—CₑR^{#}′,

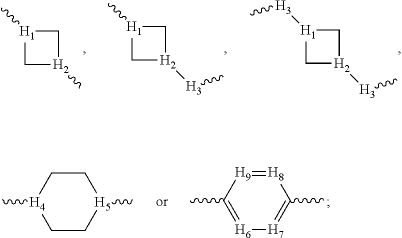

wherein one of C_{c}R^{#}R′, C_{d}R^{#}R^{#}′ or CₑR^{#}R^{#}′, or both of C_{c}R^{#}R′ and CₑR^{#}R^{#}′ can be replaced by O, S(O)ₚ, S(O)(═NR*) or NR^{#}, and when one of C_{c}R^{#}R^{#}′, C_{d}R^{#}R^{#}′ or CₑR^{#}R^{#}′ is replaced by O, S or NR^{#}, the other adjacent one or two of C_{c}R^{#}R^{#}′, C_{d}R^{#}R^{#}′ or CₑR^{#}R^{#}′ can also be replaced by S(O)_{q};

or two E moieties can be taken together to form —CH₂CH₂OCH₂CH₂—, —OCH₂CH₂CH₂CH₂—, —CH₂CH₂CH₂CH₂O—,

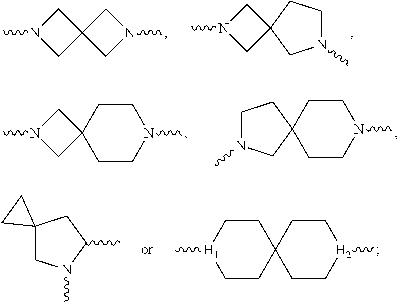

wherein [symbol] represents the point of attachment to L₁ or L₂;

[symbol]

H₁ and H₂ are N or C atom, H₃ is O, S, N or C atom, and H₁ and H₃, H₂ and H₃ are not heteroatoms at the same time;

H₄ and H₅ are N or C atom;

H₆, H₇, H₈ and H₉ are C or N atom;

p is 0, 1 or 2;

q is 1 or 2;

R* is H, C₁₋₆ alkyl, C₁₋₆ haloalkyl, C₂₋₆ alkenyl, C₂₋₆ alkynyl, C₃₋₁₀ cycloalkyl, C₃₋₁₀ halocycloalkyl, 3- to 10-membered heterocyclyl, C₆₋₁₀ aryl or 5- to 14-membered heteroaryl;

R^{#} is H, halogen, C₁₋₆ alkyl, C₁₋₆ haloalkyl, C₂₋₆ alkenyl, C₂₋₆ alkynyl, C₃₋₁₀ cycloalkyl, C₃₋₁₀ halocycloalkyl, 3- to 10-membered heterocyclyl, C₆₋₁₀ aryl or 5- to 14-membered heteroaryl;

R^{#}′ is H, halogen, C₁₋₆ alkyl, C₁₋₆ haloalkyl, C₂₋₆ alkenyl, C₂₋₆ alkynyl, C₃₋₁₀ cycloalkyl, C₃₋₁₀ halocycloalkyl, 3- to 10-membered heterocyclyl, C₆₋₁₀ aryl or 5- to 14-membered heteroaryl;

or, R^{#} and R^{#} on adjacent atoms can be taken together to form bond, and R^{#}′ and R^{#}′ on adjacent atoms can be taken together to form bond;

or, R^{#} and R^{#}′ on the same or different atoms can be taken together to form ═O, or C₃₋₇ cycloalkyl, 4- to 8-membered heterocyclyl, C₆₋₁₀ aryl or 5- to 6-membered heteroaryl, wherein the C₃₋₇ cycloalkyl, 4- to 8-membered heterocyclyl, C₆₋₁₀ aryl or 5- to 6-membered heteroaryl is optionally substituted with Rₓ, and the Rₓ is H, CN, halogen, C₁₋₆ alkyl or C₁₋₆ haloalkyl;

m is 0, 1, 2, 3, 4, 5, 6, 7, 8, 9 or 10;

Rₛ₁ is selected from H, CN, halogen, —(CH₂)₀₋₅—OR″, —(CH₂)₀₋₅—NR″R″′, C₁₋₆ alkyl, C₁₋₆ haloalkyl, —(CH₂)₀₋₅-4- to 8-membered heterocyclyl, C₂₋₆ alkenyl, C₂₋₆ alkynyl, —(CH₂)₀₋₅—C₃₋₇ cycloalkyl, —(CH₂)₀₋₅—C₃₋₁₀ halocycloalkyl, —(CH₂)₀₋₅—C₆₋₁₀ aryl, —(CH₂)₀₋₅-5- to 14-membered heteroaryl, —C(O)R_{W}, —S(O)R_{W} or —S(O)₂R_{W};

s1 is 0, 1, 2 or 3;

R′ is selected from H, —C(O)—C₁₋₆ alkyl, —C(O)—C₁₋₆ haloalkyl, —C(O)—C₂₋₆ alkenyl or —C(O)—C₆₋₁₀ aryl;

L is bond, —O— or —NR—;

wherein R is H or C₁₋₆ alkyl;

Z is —N═ or —C(R^{#})═;

T is selected from bond, C₂₋₆ heteroalkylene, 4- to 12-membered heterocyclylene, or 5- to 6-membered heterocyclylene, wherein the 5- to 6-membered heterocyclylene is substituted with 5- to 6-membered heterocyclylene;

R₁ is selected from H, halogen, cyano, C₁₋₆ alkyl, C₃₋₇ cycloalkyl, 4- to 8-membered heterocyclyl, C₁₋₆ haloalkyl, C₂₋₆ alkenyl or C₂₋₆ alkynyl;

or, when L is —NR—, R₁ and R are taken together with the atoms to which they are attached to form optionally substituted 5- to 6-membered heterocyclyl, wherein the substituent is selected from halogen, oxo, C₁₋₆ alkyl, C₁₋₆ haloalkyl, C₆₋₁₀ aryl, wherein the C₆₋₁₀ aryl is mono- or poly-substituted with halogen;

R₂ is H, halogen, hydroxyl, amino, C₁₋₆ alkyl or C₁₋₆ haloalkyl;

or R₁ and R₂ are taken together with the atoms to which they are attached to form 5- to 6-membered heterocyclyl or 5- to 6-membered heteroaryl;

R₃ is selected from H, —O—C₁₋₆ alkyl or —O—C₁₋₆ haloalkyl;

R₄ is selected from H, halogen, C₁₋₆ alkyl, C₁₋₆ haloalkyl, —NHC(O)—C₁₋₆ alkyl or —NHC(O)—C₂₋₆ alkenyl;

if the above groups are H or H-containing groups, the one or more H atom(s) may be substituted with D atom(s);

the groups containing OH, NH, NH₂, CH, CH₂, or CH₃ in L₁, E, L₂, and T, or the above alkyl, alkylene, haloalkyl, alkenyl, alkynyl, cycloalkyl, halocycloalkyl, heterocyclyl, aryl, and heteroaryl are each optionally substituted with 1, 2, 3 or more R^{s} at each occurrence, and the R^{s} is independently selected from the following groups at each occurrence: halogen, hydroxyl, amino, cyano, nitro, C₁₋₆ alkyl, C₁₋₆ haloalkyl, C₂₋₆ alkenyl, C₂₋₆ alkynyl, C₃₋₁₀ cycloalkyl, C₃. 10 halocycloalkyl, 3- to 10-membered heterocyclyl, C₆₋₁₀ aryl, 5- to 14-membered heteroaryl, C₆₋₁₂ aralkyl, —OR^{a′}, —OC(O)R^{a′}, —C(O)R^{a′}, —C(O)OR^{a′}, —C(O)NR^{a′}R^{b′}, —S(O)R^{a′}, —S(O)ₙOR^{a′}, —S(O)ₙNR^{a′}R^{b′}, —NR^{a′}R^{b′}, —NR^{a′}C(O)R^{b′}, —NR^{a′}—C(O)OR^{b′}, —NR^{a′}—S(O)ₙ—R^{b′}, —NR^{a′}C(O)NR^{a′}R^{b′}, —C₁₋₆ alkylene-R^{a′}, —C₁₋₆ alkylene-OR^{a′}, —C₁₋₆ alkylene-OC(O)R^{a′}, —C₁₋₆ alkylene-C(O)OR^{a′}, —C₁ 6 alkylene-S(O)ₙR^{a′}, —C₁₋₆ alkylene-S(O)ₙOR^{a′}, —C₁₋₆ alkylene-OC(O)NR^{a′}R′, —C₁₋₆ alkylene-C(O)NR^{a′}R′, —C₁₋₆ alkylene-NR^{a′}—C(O)NR^{a′}R′, —C₁₋₆ alkylene-OS(O)ₙR^{a′}, —C₁₋₆ alkylene-S(O)ₙNR^{a′}R′, —C₁₋₆ alkylene-NR^{a′}—S(O)ₙNR^{a′}R′, —C₁₋₆ alkylene-NR^{a′}R′ and —O—C₁₋₆ alkylene-NR^{a′}R′, and wherein the hydroxyl, amino, alkyl, alkylene, cycloalkyl, heterocyclyl, aryl, heteroaryl and aralkyl described with respect to the substituent R^{s} are further optionally substituted with 1, 2, 3 or more substituents independently selected from: halogen, OH, amino, cyano, nitro, C₁₋₆ alkyl, C₁₋₆ haloalkyl, C₁₋₆ alkyl hydroxyl, C₃₋₆ cycloalkyl, 3- to 10-membered heterocyclyl, C₆₋₁₀ aryl, 5- to 14-membered heteroaryl and C₆₋₁₂ aralkyl;

n is independently 1 or 2 at each occurrence;

each of R^{a′} and R^{b′} is independently selected from H, C₁₋₆ alkyl, C₂₋₆ alkenyl, C₂₋₆ alkynyl, C₁₋₆ alkyl-O—, C₁₋₆ alkyl-S—, C₃₋₁₀ cycloalkyl, 3- to 10-membered heterocyclyl, C₆₋₁₀ aryl, 5- to 14-membered heteroaryl and C₆₋₁₂ aralkyl at each occurrence.

In another specific embodiment, the present disclosure relates to a compound of general formula (X), or a pharmaceutically acceptable salt, enantiomer, diastereomer, racemate, solvate, hydrate, polymorph, prodrug, or isotopic variant thereof, or mixture thereof:

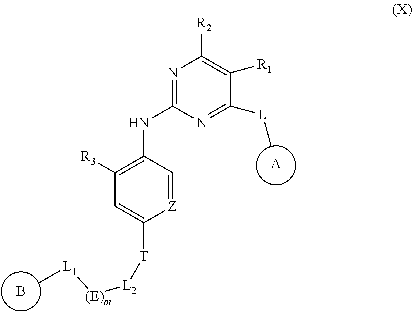

wherein

ring A is selected from the following optionally substituted groups: C₃₋₇ cycloalkyl, 4- to 8-membered heterocyclyl, C₆₋₁₀ aryl or 5- to 10-membered heteroaryl, wherein the substituent is selected from halogen, —CN, C₁₋₆ alkyl, C₁₋₆ haloalkyl, C₂₋₆ alkenyl, C₃₋₇ cycloalkyl, 4- to 8-membered heterocyclyl, —P(O)(C₁₋₆ alkyl)₂, —P(O)(C₂₋₆ alkenyl)₂, —O—C₁₋₆ alkyl, —O—C₁₋₆ haloalkyl, —O—C₂₋₆ alkenyl, —O—C₃₋₇ cycloalkyl, —O-4- to 8-membered heterocyclyl, —NH—C₁₋₆ alkyl, —NH—C₂₋₆ alkenyl, —NH—C₃₋₇ cycloalkyl, —NH-4- to 8-membered heterocyclyl, —C(O)—C₁₋₆ alkyl, —C(O)—C₂₋₆ alkenyl, —C(O)—C₃₋₇ cycloalkyl, —C(O)-4- to 8-membered heterocyclyl, —S(O)₂—C₁₋₆ alkyl, —S(O)₂—C₂₋₆ alkenyl, —S(O)₂—C₃₋₇ cycloalkyl, —S(O)₂-4- to 8-membered heterocyclyl, —NHC(O)—C₁₋₆ alkyl, —NHC(O)—C₂₋₆ alkenyl, —NHC(O)—C₃₋₇ cycloalkyl, —NHC(O)-4- to 8-membered heterocyclyl, —NHS(O)₂—C₁₋₆ alkyl, —NHS(O)₂—C₂₋₆ alkenyl, —NHS(O)₂—C₃₋₇ cycloalkyl or —NHS(O)₂-4- to 8-membered heterocyclyl;

ring B is selected from the following groups:

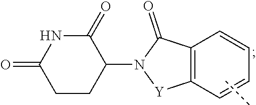

wherein Y is —CH₂— or —C(O)—;

R′ is selected from H, —C(O)—C₁₋₆ alkyl, —C(O)—C₁₋₆ haloalkyl, —C(O)—C₂₋₆ alkenyl or —C(O)—C₆₋₁₀ aryl;

[symbol]

[symbol] represents the point of attachment to L₁;

L is bond, —O— or —NR—;

wherein R is H or C₁₋₆ alkyl;

Z is —N═ or —C(R^{#})═;

T is selected from bond, C₂₋₆ heteroalkylene, 4- to 12-membered heterocyclylene, or 5- to 6-membered heterocyclylene, wherein the 5- to 6-membered heterocyclylene is substituted with 5- to 6-membered heterocyclylene;

L₁ is selected from bond, —O—, —NH—, —CH₂—, —C(O)—, —CH₂CH₂—, —CH═CH—, —C≡C—, —OCH₂—, —CH₂O—, —NHCH₂—, —CH₂NH—, —C(O)CH₂—, —CH₂C(O)—, —OC(O)—, —C(O)O—, —NHC(O)— or —C(O)NH—;

E is independently selected from —CH₂CH₂CH₂—, —CH₂CH₂C(O)—, —CH₂C(O)CH₂—, —C(O)CH₂CH₂—, —C(O)CH═CH—, —C(O)C≡C—, —CH₂CH₂O—, —CH₂OCH₂—, —OCH₂CH₂—, —C(O)CH₂O—, —OCH₂C(O)—, —CH₂C(O)O—, —OC(O)CH₂—, —C(O)OCH₂—, —CH₂OC(O)—, —CH₂CH₂NH—, —CH₂NHCH₂—, —NHCH₂CH₂—, —C(O)CH₂NH—, —NHCH₂C(O)—, —CH₂C(O)NH—, —NHC(O)CH₂—, —C(O)NHCH₂—, —CH₂NHC(O)—,

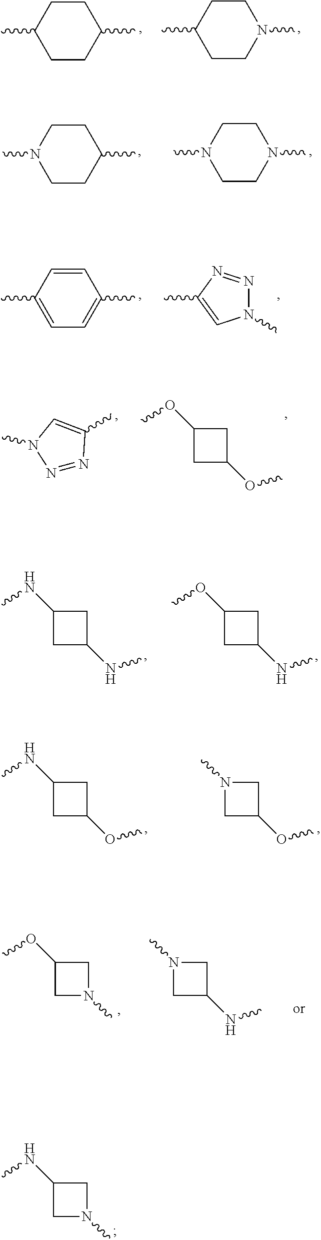

wherein [symbol] represents the point of attachment to L₁ or L₂;

m is 0, 1, 2, 3, 4, 5, 6, 7, 8, 9 or 10;

L₂ is selected from bond, —CH₂—, —CH₂CH₂—, —OCH₂—, —NHCH₂—, —OC(O)—, —NHC(O)—, —CH₂C(O)— or —C(O)CH₂—;

R₁ is selected from H, halogen, cyano, C₁₋₆ alkyl, C₃₋₇ cycloalkyl, 4- to 8-membered heterocyclyl, C₁₋₆ haloalkyl, C₂₋₆ alkenyl or C₂₋₆ alkynyl;

or, when L is —NR—, R₁ and R are taken together with the atoms to which they are attached to form optionally substituted 5- to 6-membered heterocyclyl, wherein the substituent is selected from halogen, oxo, C₁₋₆ alkyl, C₁₋₆ haloalkyl, or C₆₋₁₀ aryl, wherein the C₆₋₁₀ aryl is mono- or poly-substituted with halogen;

R₂ is H, halogen, hydroxyl, amino, C₁₋₆ alkyl or C₁₋₆ haloalkyl;

or R₁ and R₂ are taken together with the atoms to which they are attached to form 5- to 6-membered heterocyclyl or 5- to 6-membered heteroaryl;

R₃ is selected from H, —O—C₁₋₆ alkyl or —O—C₁₋₆ haloalkyl;

R₄ is selected from H, halogen, C₁₋₆ alkyl, C₁₋₆ haloalkyl, —NHC(O)—C₁₋₆ alkyl or —NHC(O)—C₂₋₆ alkenyl;

the groups containing OH, NH, NH₂, CH, CH₂, or CH₃ in L₁, E, L₂, and T, or the above alkyl, alkylene, alkenyl, alkynyl, cycloalkyl, heterocyclyl, aryl, and heteroaryl are each optionally substituted with 1, 2, 3 or more R^{s} at each occurrence, and the R^{s} is independently selected from the following groups at each occurrence: halogen, hydroxyl, amino, cyano, nitro, C₁₋₆ alkyl, C₁₋₆ haloalkyl, C₂₋₆ alkenyl, C₂₋₆ alkynyl, C₃₋₁₀ cycloalkyl, C₃₋₁₀ halocycloalkyl, 3- to 10-membered heterocyclyl, C₆₋₁₀ aryl, 5- to 14-membered heteroaryl, C₆₋₁₂ aralkyl, —OR′, —OC(O)R^{a}, —C(O)R^{a}, —C(O)OR^{a}, —C(O)NR^{a}R^{b}, —S(O)ₙR^{a}, —S(O)ₙOR^{a}, —S(O)NR^{a}R^{b}, —NR^{a}R^{b}, —NR^{a}C(O)R^{b}, —NR^{a}—C(O)OR^{b}, —NR^{a}—S(O)ₙ—R^{b}, —NR^{a}C(O)NR^{a}R^{b}, —C₁₋₆ alkylene-R^{a}, —C₁₋₆ alkylene-OR^{a}, —C₁₋₆ alkylene-OC(O)R^{a}, —C₁₋₆ alkylene-C(O)OR^{a}, —C₁₋₆ alkylene-S(O)ₙR^{a}, —C₁₋₆ alkylene-S(O)ₙOR^{a}, —C₁₋₆ alkylene-OC(O)NR^{a}R^{b}, —C₁₋₆ alkylene-C(O)NR^{a}R^{b}, —C₁₋₆ alkylene-NR^{a}—C(O)NR^{a}R^{b}, —C₁₋₆ alkylene-OS(O)ₙR^{a}, —C₁₋₆ alkylene-S(O)ₙNR^{a}R^{b}, —C₁₋₆ alkylene-NR^{a}—S(O)ₙNR^{a}R^{b}, —C₁₋₆ alkylene-NR^{a}R^{b} and —O—C₁₋₆ alkylene-NR^{a}R^{b}, and wherein the halogen, hydroxyl, amino, alkyl, alkylene, cycloalkyl, heterocyclyl, aryl, heteroaryl and aralkyl described with respect to the substituent R^{s} are further optionally substituted with 1, 2, 3 or more substituents independently selected from: halogen, OH, amino, cyano, nitro, C₁₋₆ alkyl, C₁₋₆ haloalkyl, C₁₋₆ alkyl hydroxyl, C₃₋₆ cycloalkyl, 3- to 10-membered heterocyclyl, C₆₋₁₀ aryl, 5- to 14-membered heteroaryl and C₆₋₁₂ aralkyl;

n is independently 1 or 2 at each occurrence;

R^{a} and R^{b} are independently selected from H, C₁₋₆ alkyl, C₂₋₆ alkenyl, C₂₋₆ alkynyl, C₁. 6 alkyl-O—, C₁₋₆ alkyl-S—, C₃₋₁₀ cycloalkyl, 3- to 10-membered heterocyclyl, C₆₋₁₀ aryl, 5- to 14-membered heteroaryl and C₆₋₁₂ aralkyl at each occurrence.

In a more specific embodiment, the present disclosure provides a compound of formula (I) or (I-G), or a pharmaceutically acceptable salt, enantiomer, diastereomer, racemate, solvate, hydrate, polymorph, prodrug, or isotopic variant thereof, or mixture thereof:

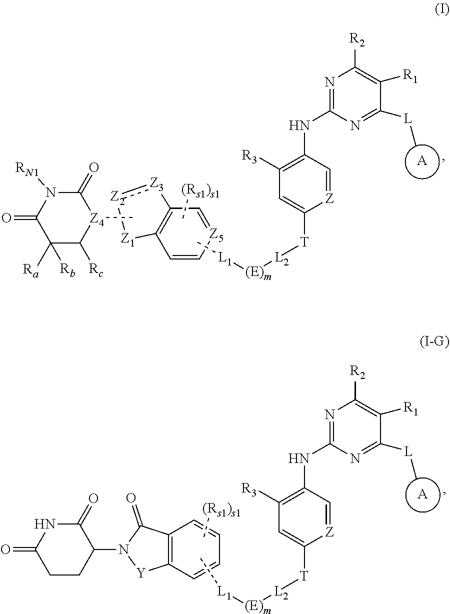

wherein each group is as defined above.

In a more specific embodiment, the present disclosure provides a compound of formula (I) or (I-G), or a pharmaceutically acceptable salt, enantiomer, diastereomer, racemate, solvate, hydrate, polymorph, prodrug, or isotopic variant thereof, or mixture thereof:

wherein

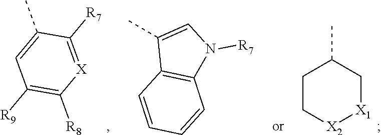

ring A is the following groups:

wherein

R₇ is selected from C₁₋₆ alkyl, C₁₋₆ haloalkyl, C₂₋₆ alkenyl, C₃₋₇ cycloalkyl, —P(O)(C₁₋₆ alkyl)₂ or —P(O)(C₂₋₆ alkenyl)₂;

R₈ is selected from H, —NH—C₁₋₆ alkyl, —NH—C₂₋₆ alkenyl, —NH—C₃₋₇ cycloalkyl, —NH-4- to 8-membered heterocyclyl, —NHC(O)—C₁₋₆ alkyl, —NHC(O)—C₂₋₆ alkenyl, —NHC(O)—C₃₋₇ cycloalkyl, —NHC(O)-4- to 8-membered heterocyclyl, —NHS(O)₂—C₁₋₆ alkyl or —NHS(O)₂—C₃₋₇ cycloalkyl;

R₉ is selected from H, halogen, —CN, C₁₋₆ haloalkyl, —O—C₁₋₆ alkyl, —O—C₁₋₆ haloalkyl, C₃₋₇ cycloalkyl, —O—C₃₋₇ cycloalkyl, C₁₋₆ alkyl, —NH—C₁₋₆ alkyl, —NH—C₂₋₆ alkenyl, —NH—C₃₋₇ cycloalkyl, —NH-4- to 8-membered heterocyclyl, —NHC(O)—C₁₋₆ alkyl, —NHC(O)—C₂₋₆ alkenyl, —NHC(O)—C₃₋₇ cycloalkyl, —NHC(O)-4- to 8-membered heterocyclyl, —NHS(O)₂—C₁₋₆ alkyl or —NHS(O)₂—C₃₋₇ cycloalkyl;

X is —C(Rₓ)═ or —N═;

wherein Rₓ is H, or Rₓ and Rₛ are taken together with the C atoms to which they are attached to form 5- to 6-membered heterocyclyl or 5- to 6-membered heteroaryl; alternatively, Rₓ and R₈ are taken together with the C atoms to which they are attached to form 5- to 6-membered heteroaryl, alternatively pyrazinyl;

X₁ is —CH(R_{X1})— or —N(R_{X1})—;

X₂ is —CH(Rₓ₂)— or —N(Rₓ₂)—;

wherein R_{X1} is selected from H, C₁₋₆ alkyl, C₃₋₇ cycloalkyl, —O—C₁₋₆ alkyl, —O—C₃₋₇ cycloalkyl, —NH—C₁₋₆ alkyl, —NH—C₃₋₇ cycloalkyl, —S(O)₂—C₁₋₆ alkyl, —S(O)₂—C₃₋₇ cycloalkyl, —NHS(O)₂—C₁₋₆ alkyl, —NHS(O)₂—C₃₋₇ cycloalkyl, —C(O)—C₁₋₆ alkyl, —C(O)—C₂₋₆ alkenyl, —C(O)—C₃₋₇ cycloalkyl, —NHC(O)—C₁₋₆ alkyl, —NHC(O)—C₃₋₇ cycloalkyl or —C(O)-4- to 8-membered heterocyclyl; R_{X2} is selected from H, C₁₋₆ alkyl, C₃₋₇ cycloalkyl, —O—C₁₋₆ alkyl, —O—C₃₋₇ cycloalkyl, —NH—C₁₋₆ alkyl, —NH—C₃₋₇ cycloalkyl, —C(O)—C₁₋₆ alkyl, —C(O)—C₃₋₇ cycloalkyl, —C(O)-4- to 8-membered heterocyclyl, —S(O)₂—C₁₋₆ alkyl, —S(O)₂—C₃₋₇ cycloalkyl, —NHS(O)₂—C₁. 6 alkyl, —NHS(O)₂—C₃₋₇ cycloalkyl, —NHC(O)—C₁₋₆ alkyl, —NHC(O)—C₂₋₆ alkenyl, —NHC(O)—C₃₋₇ cycloalkyl or —NHC(O)-4- to 8-membered heterocyclyl;

[symbol] represents the point of attachment to L;

other groups are as defined above.

In a more specific embodiment, the present disclosure provides a compound of formula (I) or (I-G), or a pharmaceutically acceptable salt, enantiomer, diastereomer, racemate, solvate, hydrate, polymorph, prodrug, or isotopic variant thereof, or mixture thereof:

wherein

T is bond,

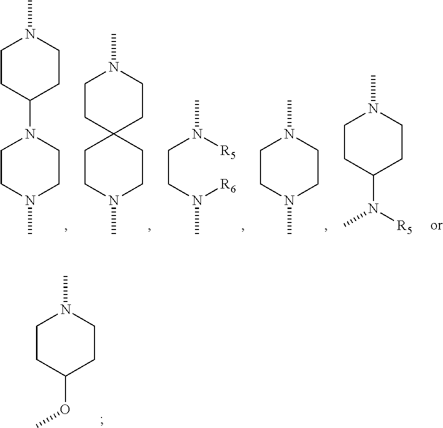

wherein

R₅ is H or C₁₋₆ alkyl;

R₆ is H or C₁₋₆ alkyl;

or R₅ and R₆ are connected to form C₁₋₆ alkylene;

[symbol] represents the point of attachment to parent core or L₂;

other groups are as defined above.

In a more specific embodiment, the present disclosure provides a compound of formula (I) or (I-G), or a pharmaceutically acceptable salt, enantiomer, diastereomer, racemate, solvate, hydrate, polymorph, prodrug, or isotopic variant thereof, or mixture thereof:

wherein

when L is —NR—, R₁ and R are taken together to form the following groups: —C(O)N(R_{N})C(O)—or —C(C₁₋₆ alkyl)═C(R_{N})C(O)—;

wherein

R_{N} is selected from C₁₋₆ alkyl or

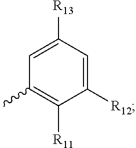

R₁₁ is H or halogen;

R₁₂ is H or halogen;

R₁₃ is H or halogen;

[symbol] represents the point of attachment;

other groups are as defined above.

In a more specific embodiment, the present disclosure provides a compound of formula (I) or (I-G), or a pharmaceutically acceptable salt, enantiomer, diastereomer, racemate, solvate, hydrate, polymorph, prodrug, or isotopic variant thereof, or mixture thereof:

wherein R₁ and R₂ are taken together with the atoms to which they are attached to form 5- to 6-membered heteroaryl; alternatively form pyrrolyl; other groups are as defined above.

Ring A

In a specific embodiment, ring A is optionally substituted C₃₋₇ cycloalkyl; in another specific embodiment, ring A is optionally substituted 4- to 8-membered heterocyclyl; in another specific embodiment, ring A is optionally substituted C₆₋₁₀ aryl; in another specific embodiment, ring A is optionally substituted 5- to 10-membered heteroaryl; in another specific embodiment, ring A is

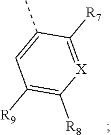

in another specific embodiment, ring A is

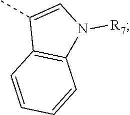

in another specific embodiment, ring A is

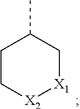

In a specific embodiment described above, the substituent of ring A is halogen; in another specific embodiment described above, the substituent of ring A is —CN; in another specific embodiment described above, the substituent of ring A is C₁₋₆ alkyl; in another specific embodiment described above, the substituent of ring A is C₁₋₆ haloalkyl; in another specific embodiment described above, the substituent of ring A is C₂₋₆ alkenyl; in another specific embodiment described above, the substituent of ring A is C₃₋₇ cycloalkyl; in another specific embodiment described above, the substituent of ring A is 4- to 8-membered heterocyclyl; in another specific embodiment described above, the substituent of ring A is —P(O)(C₁₋₆ alkyl)₂; in another specific embodiment described above, the substituent of ring A is —P(O)(C₂₋₆ alkenyl)₂; in another specific embodiment described above, the substituent of ring A is —O—C₁₋₆ alkyl; in another specific embodiment described above, the substituent of ring A is —O—C₁₋₆ haloalkyl; in another specific embodiment described above, the substituent of ring A is —O—C₂₋₆ alkenyl; in another specific embodiment described above, the substituent of ring A is —O—C₃₋₇ cycloalkyl; in another specific embodiment described above, the substituent of ring A is —O-4- to 8-membered heterocyclyl; in another specific embodiment described above, the substituent of ring A is —NH—C₁₋₆ alkyl; in another specific embodiment described above, the substituent of ring A is —NH—C₂₋₆ alkenyl; in another specific embodiment described above, the substituent of ring A is —NH—C₃₋₇ cycloalkyl; in another specific embodiment described above, the substituent of ring A is —NH-4- to 8-membered heterocyclyl; in another specific embodiment described above, the substituent of ring A is —C(O)—C₁₋₆ alkyl; in another specific embodiment described above, the substituent of ring A is —C(O)—C₂₋₆ alkenyl; in another specific embodiment described above, the substituent of ring A is —C(O)—C₃₋₇ cycloalkyl; in another specific embodiment described above, the substituent of ring A is —C(O)-4- to 8-membered heterocyclyl; in another specific embodiment described above, the substituent of ring A is —S(O)₂—C₁₋₆ alkyl; in another specific embodiment described above, the substituent of ring A is —S(O)₂—C₂₋₆ alkenyl; in another specific embodiment described above, the substituent of ring A is —S(O)₂—C₃₋₇ cycloalkyl; in another specific embodiment described above, the substituent of ring A is —S(O)₂-4- to 8-membered heterocyclyl; in another specific embodiment described above, the substituent of ring A is —NHC(O)—C₁₋₆ alkyl; in another specific embodiment described above, the substituent of ring A is —NHC(O)—C₂₋₆ alkenyl; in another specific embodiment described above, the substituent of ring A is —NHC(O)—C₃₋₇ cycloalkyl; in another specific embodiment described above, the substituent of ring A is —NHC(O)-4- to 8-membered heterocyclyl; in another specific embodiment described above, the substituent of ring A is —NHS(O)₂—C₁₋₆ alkyl; in another specific embodiment described above, the substituent of ring A is —NHS(O)₂—C₂₋₆ alkenyl; in another specific embodiment described above, the substituent of ring A is —NHS(O)₂—C₃₋₇ cycloalkyl; in another specific embodiment described above, the substituent of ring A is —NHS(O)₂-4- to 8-membered heterocyclyl.

L

In a specific embodiment, L is bond; in another specific embodiment, L is —O—; in another specific embodiment, L is —NR—.

Y

In a specific embodiment, Y is —CH₂—; in another specific embodiment, Y is —C(O)—.

Z

In a specific embodiment, Z is —N═; in another specific embodiment, Z is —C(R₄)═.

T

In a specific embodiment, T is C₂₋₆ heteroalkylene; in another specific embodiment, T is 4- to 12-membered heterocyclylene; in another specific embodiment, T is 5- to 6-membered heterocyclylene substituted with 5- to 6-membered heterocyclylene; in another specific embodiment, T is

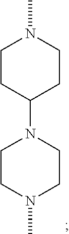

in another specific embodiment, T is

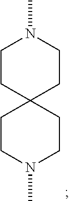

in another specific embodiment, T is

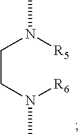

in another specific embodiment, T is

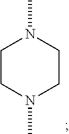

in another specific embodiment, T is bond.

[symbol]

In a specific embodiment, [symbol] represents single bond; in another specific embodiment, [symbol] represents double bond.

Z₁

In a specific embodiment, Z₁ is O atom; in another specific embodiment, Z₁ is S atom; in another specific embodiment, Z₁ is N atom; in another specific embodiment, Z₁ is C atom; in another specific embodiment, Z₁ is substituted with one R_{Z1}; in another specific embodiment, Z₁ is substituted with two R_{Z1}; in another specific embodiment, Z₁ is absent.

Z₂

In a specific embodiment, Z₂ is O atom; in another specific embodiment, Z₂ is S atom; in another specific embodiment, Z₂ is N atom; in another specific embodiment, Z₂ is C atom; in another specific embodiment, Z₂ is substituted with one R_{Z2}; in another specific embodiment, Z₂ is substituted with two R_{Z2}.

Z₃

In a specific embodiment, Z₃ is O atom; in another specific embodiment, Z₃ is S atom; in another specific embodiment, Z₃ is N atom; in another specific embodiment, Z₃ is C atom; in another specific embodiment, Z₃ is substituted with one R_{Z3}; in another specific embodiment, Z₃ is substituted with two R_{Z3}.

In a specific embodiment, Z₁, Z₂ and Z₃ are all absent.

Z₄

In a specific embodiment, Z₄ is N; in another specific embodiment, Z₄ is CR_{Z4}.

Z₅

In a specific embodiment, Z₅ is N; in another specific embodiment, Z₅ is CR_{Z5}.

R^{a}, R^{b} and R,

In a specific embodiment, R^{a} is H; in another specific embodiment, R^{a} is halogen; in another specific embodiment, R^{a} is OR′; in another specific embodiment, R^{a} is NR′R″; in another specific embodiment, R^{a} is C₁₋₆ alkyl; in another specific embodiment, R^{a} is C₁₋₆ haloalkyl.

In a specific embodiment, R^{b} is H; in another specific embodiment, R^{b} is halogen; in another specific embodiment, R^{b} is OR′; in another specific embodiment, R^{b} is NR′R″; in another specific embodiment, R^{b} is C₁₋₆ alkyl; in another specific embodiment, R^{b} is C₁₋₆ haloalkyl.

In a specific embodiment, R, is H; in another specific embodiment, R, is halogen; in another specific embodiment, R, is OR′; in another specific embodiment, R, is NR′R″; in another specific embodiment, R, is C₁₋₆ alkyl; in another specific embodiment, R, is C₁₋₆ haloalkyl.

In a specific embodiment, R^{a} and R^{b} are taken together with the carbon atom to which they are attached to form C═O; in another specific embodiment, R^{a} and R^{b} are taken together with the carbon atom to which they are attached to form C₃₋₇ cycloalkyl; in another specific embodiment, R^{a} and R^{b} are taken together with the carbon atom to which they are attached to form 4- to 8-membered heterocyclyl; in another specific embodiment, R^{a} and R, are taken together to form bond; in another specific embodiment, R^{a} and R, are taken together with the carbon atoms to which they are attached to form C₃₋₇ cycloalkyl or 4- to 8-membered heterocyclyl.

R_{Z1}

In a specific embodiment, R_{Z1} is absent; in another specific embodiment, R_{Z1} is H; in another specific embodiment, R_{Z1} is CN; in another specific embodiment, R_{Z1} is halogen; in another specific embodiment, R_{Z1} is —(CH₂)₀₋₅—OR′; in another specific embodiment, R_{Z1} is —(CH₂)₀₋₅—NR′R″; in another specific embodiment, R_{Z1} is C₁₋₆ alkyl; in another specific embodiment, R_{Z1} is C₁₋₆ haloalkyl; in another specific embodiment, R_{Z1} is —(CH₂)₀₋₅—C₃₋₇ cycloalkyl; in another specific embodiment, R_{Z1} is —(CH₂)₀₋₅-4- to 8-membered heterocyclyl; in another specific embodiment, two R_{Z1} are taken together with Z₁ to form C═O; in another specific embodiment, two R_{Z1} are taken together with Z₁ to form C₃₋₇ cycloalkyl; in another specific embodiment, two R_{Z1} are taken together with Z₁ to form 4- to 8-membered heterocyclyl.

R_{Z2}

In a specific embodiment, R_{Z2} is absent; in another specific embodiment, R_{Z2} is H; in another specific embodiment, R_{Z2} is CN; in another specific embodiment, R_{Z2} is halogen; in another specific embodiment, R_{Z2} is —(CH₂)₀₋₅—OR′; in another specific embodiment, R_{Z2} is —(CH₂)₀₋₅—NR′R″; in another specific embodiment, R_{Z2} is C₁₋₆ alkyl; in another specific embodiment, R_{Z2} is C₁₋₆ haloalkyl; in another specific embodiment, R_{Z2} is —(CH₂)₀₋₅—C₃₋₇ cycloalkyl; in another specific embodiment, R_{Z2} is —(CH₂)₀₋₅-4- to 8-membered heterocyclyl; in another specific embodiment, two R_{Z2} are taken together with Z₂ to form C═O; in another specific embodiment, two R_{Z2} are taken together with Z₂ to form C₃₋₇ cycloalkyl; in another specific embodiment, two R_{Z2} are taken together with Z₂ to form 4- to 8-membered heterocyclyl.

R_{Z3}

In a specific embodiment, R_{Z3} is absent; in another specific embodiment, R_{Z3} is H; in another specific embodiment, R_{Z3} is CN; in another specific embodiment, R_{Z3} is halogen; in another specific embodiment, R_{Z3} is —(CH₂)₀₋₅—OR′; in another specific embodiment, R_{Z3} is —(CH₂)₀₋₅—NR′R″; in another specific embodiment, R_{Z3} is C₁₋₆ alkyl; in another specific embodiment, R_{Z3} is C₁₋₆ haloalkyl; in another specific embodiment, R_{Z3} is —(CH₂)₀₋₅—C₃₋₇ cycloalkyl; in another specific embodiment, R_{Z3} is —(CH₂)₀₋₅-4- to 8-membered heterocyclyl; in another specific embodiment, two R_{Z3} are taken together with Z₃ to form C═O; in another specific embodiment, two R_{Z3} are taken together with Z₃ to form C₃₋₇ cycloalkyl; in another specific embodiment, two R_{Z3} are taken together with Z₃ to form 4- to 8-membered heterocyclyl.

R_{Z4}

In a specific embodiment, R_{Z4} is H; in another specific embodiment, R_{Z4} is CN; in another specific embodiment, R_{Z4} is halogen; in another specific embodiment, R_{Z4} is —(CH₂)₀₋₅—OR′; in another specific embodiment, R_{Z4} is —(CH₂)₀₋₅—NR′R″; in another specific embodiment, R_{Z4} is C₁₋₆ alkyl; in another specific embodiment, R_{Z4} is C₁₋₆ haloalkyl.

R_{Z5}

In a specific embodiment, R_{Z5} is H; in another specific embodiment, R_{Z5} is CN; in another specific embodiment, R_{Z5} is halogen; in another specific embodiment, R_{Z5} is —(CH₂)₀₋₅—OR′; in another specific embodiment, R_{Z5} is —(CH₂)₀₋₅—NR′R″; in another specific embodiment, R_{Z5} is C₁₋₆ alkyl; in another specific embodiment, R_{Z5} is C₁₋₆ haloalkyl; in another specific embodiment, R_{Z5} is —(CH₂)₀₋₅—C₃₋₇ cycloalkyl; in another specific embodiment, Rzs is —(CH₂)₀₋₅-4- to 8-membered heterocyclyl.

In a specific embodiment, the ring in which Z₄ is located is absent.

L₁

In a specific embodiment, L₁ is bond; in another specific embodiment, L₁ is —O—; in another specific embodiment, L₁ is —S(O)ₚ—; in another specific embodiment, L₁ is —S(O)(═NR*)—; in another specific embodiment, L₁ is —NR^{#}—; in another specific embodiment, L₁ is —CR^{#}R^{#}′—; in another specific embodiment, L₁ is —CₐR^{#}R^{#}′—C_{b}R^{#}R^{#}′—; in another specific embodiment, L₁ is —N═S(O)(R*)—; in another specific embodiment, L₁ is —S(O)(R*)═N—.

L₂

In a specific embodiment, L₂ is bond; in another specific embodiment, L₂ is —O—; in another specific embodiment, L₂ is —S(O)ₚ—; in another specific embodiment, L₂ is —S(O)(═NR*)—; in another specific embodiment, L₂ is —NR^{#}—; in another specific embodiment, L₂ is —CR^{#}R^{#}′—; in another specific embodiment, L₂ is —CₐR4R^{#}′—C_{b}R^{#}R^{#}′—; in another specific embodiment, L₂ is —N═S(O)(R*)—; in another specific embodiment, L₂ is —S(O)(R*)═N—.

In another specific embodiment, one of CₐR^{#}R^{#}′ and C_{b}R^{#}R^{#}′ in L₁ or L₂ can be replaced by O, S(O)ₚ, S(O)(═NR*) or NR^{#}, and when one of CₐR^{#}R′ and C_{b}R^{#}R^{#}″ is replaced by O, S or NR^{#}, the other of CₐR^{#}R^{#}′ and C_{b}R^{#}R^{#}′ can also be replaced by S(O)_{q};

E

In a specific embodiment, E is bond; in another specific embodiment, E is —C_{c}R^{#}R^{#}′—C_{d}R^{#}R^{«}′—CₑR^{#}R^{#}; in another specific embodiment, E is

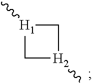

in another specific embodiment, E is

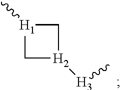

in another specific embodiment, E is

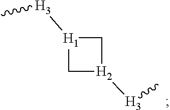

in another specific embodiment, E is

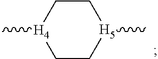

in another specific embodiment, E is

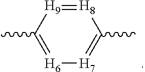

In another specific embodiment, one of C_{c}R^{#}R^{#}′, C_{d}R^{#}R^{#}′ or CₑR^{#}R^{#}′, or both of C_{c}R^{#}R^{#}′ and CₑR^{#}R^{#}′ can be replaced by O, S(O)ₚ, S(O)(═NR*) or NR^{#}, and when one of C_{c}R^{#}R^{#}′, C_{d}R^{#}R^{#}′ or CₑR^{#}R^{#}′ is replaced by O, S or NR^{#}, the other adjacent one or two of C_{c}R^{#}R^{#}′, C_{d}R^{#}R^{#}′ or CₑR^{#}R^{#}′ can also be replaced by S(O)_{q};

In another specific embodiment, two E moieties can be taken together to form —CH₂CH₂OCH₂CH₂—; in another specific embodiment, two E moieties can be taken together to form —OCH₂CH₂CH₂CH₂—; in another specific embodiment, two E moieties can be taken together to form —CH₂CH₂CH₂CH₂O—; in another specific embodiment, two E moieties can be taken together to form

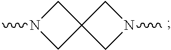

taken together to form

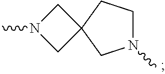

in another specific embodiment, two E moieties can be taken together to form

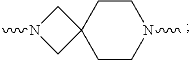

in another specific embodiment, two E moieties can be taken together to form

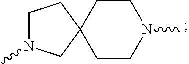

in another specific embodiment, two E moieties can be taken together to form

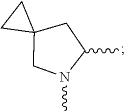

in another specific embodiment, two E moieties can be taken together to form

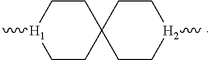

In another specific embodiment, in the embodiment of L₁, L₂ or E, R^{#} and R^{#} on adjacent atoms can be taken together to form bond, and R^{#}′ and R^{#}′ on adjacent atoms can be taken together to form bond;

In another specific embodiment, in the embodiment of L₁, L₂ or E, R^{#} and R^{#}′ on the same atom can be taken together to form ═O, or C₃₋₇ cycloalkyl, 4- to 8-membered heterocyclyl, C₆₋₁₀ aryl or 5- to 6-membered heteroaryl, each of which is optionally substituted with Rₓ group; in another specific embodiment, in the embodiment of L₁, L₂ or E, R^{#} and R^{#}′ on different atoms can be taken together to form C₃₋₇ cycloalkyl, 4- to 8-membered heterocyclyl, C₆₋₁₀ aryl or 5- to 6-membered heteroaryl, each of which is optionally substituted with Rₓ group.

m

In a specific embodiment, m is 0; in another specific embodiment, m is 1; in another specific embodiment, m is 2; in another specific embodiment, m is 3; in another specific embodiment, m is 4; in another specific embodiment, m is 5; in another specific embodiment, m is 6; in another specific embodiment, m is 7; in another specific embodiment, m is 8; in another specific embodiment, m is 9; in another specific embodiment, m is 10.

R₁

In a specific embodiment, R₁ is H; in another specific embodiment, R₁ is halogen; in another specific embodiment, R₁ is cyano; in another specific embodiment, R₁ is C₁₋₆ alkyl; in another specific embodiment, R₁ is C₃₋₇ cycloalkyl; in another specific embodiment, R₁ is 4- to 8-membered heterocyclyl; in another specific embodiment, R₁ is C₁₋₆ haloalkyl; in another specific embodiment, R₁ is C₂₋₆ alkenyl; in another specific embodiment, R₁ is C₂₋₆ alkynyl. In another specific embodiment, when L is —NR—, R₁ and R are taken together with the atoms to which they are attached to form optionally substituted 5- to 6-membered heterocyclyl, wherein the substituent is selected from halogen, oxo, C₁₋₆ alkyl, C₁₋₆ haloalkyl, C₆₋₁₀ aryl, wherein the C₆₋₁₀ aryl is mono- or poly-substituted with halogen; in another specific embodiment, R₁ and R are taken together to form —C(O)N(R_{N})C(O)—; in another specific embodiment, R₁ and R are taken together to form —C(C₁₋₆ alkyl)═C(R_{N})C(O)—.

R₂

In a specific embodiment, R₂ is H; in another specific embodiment, R₂ is halogen; in another specific embodiment, R₂ is hydroxyl; in another specific embodiment, R₂ is amino; in another specific embodiment, R₂ is C₁₋₆ alkyl; in another specific embodiment, R₂ is C₁₋₆ haloalkyl; in another specific embodiment, R₁ and R₂ are taken together with the atoms to which they are attached to form 5- to 6-membered heterocyclyl; in another specific embodiment, R₁ and R₂ are taken together with the atoms to which they are attached to form 5- to 6-membered heteroaryl; in another specific embodiment, R₁ and R₂ are taken together with the atoms to which they are attached to form pyrrolyl.

R₃

In a specific embodiment, R₃ is H; in another specific embodiment, R₃ is H—O—C₁₋₆ alkyl; in another specific embodiment, R₃ is —O—C₁₋₆ haloalkyl.

R₄

In a specific embodiment, R₄ is H; in another specific embodiment, R₄ is halogen; in another specific embodiment, R₄ is C₁₋₆ alkyl; in another specific embodiment, R₄ is C₁₋₆ haloalkyl; in another specific embodiment, R₄ is —NHC(O)—C₁₋₆ alkyl; in another specific embodiment, R₄ is —NHC(O)—C₂₋₆ alkenyl.

Any technical solution in any one of the above specific embodiments, or any combination thereof, may be combined with any technical solution in other specific embodiments or any combination thereof. For example, any technical solution of ring A or any combination thereof may be combined with any technical solution of L, Y, Z₁—Z₅, R′, T, L₁, E, m, L₂, R₁—R₄ or any combination thereof. The present disclosure is intended to include all combination of such technical solutions, which are not exhaustively listed here to save space.

In a more specific embodiment, the present disclosure provides a compound of formula (I) or (I-G), or a pharmaceutically acceptable salt, enantiomer, diastereomer, racemate, solvate, hydrate, polymorph, prodrug, or isotopic variant thereof, or mixture thereof, wherein

ring A is selected from the following optionally substituted groups: C₃₋₇ cycloalkyl, 4- to 8-membered heterocyclyl, C₆₋₁₀ aryl or 5- to 10-membered heteroaryl, wherein the substituent is selected from —F, —Cl, —Br, -Me, —OMe, —CF₃, —OCF₃, —CN, —NHMe, cyclopropyl, —P(O)Me₂, —NHC(O)CH₂CH₃, —C(O)CH═CH₂, —NHS(O)₂CH₂CH₃, —NH-cyclopropyl, —NHC(O)CH═CH₂ or —C(O)CH₂CH₃; ring A is alternatively the following groups:

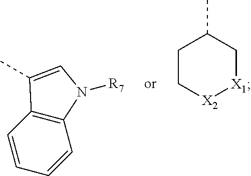

wherein

R₇ is selected from -Me, cyclopropyl or —P(O)Me₂;

R₈ is selected from H, —NHMe, —NHC(O)CH₂CH₃ or —NH-cyclopropyl;

R₉ is selected from H, —F, —Cl, —Br, -Me, —CF₃, —OMe, —OCF₃, —CN, —NHC(O)CH₂CH₃, —NHS(O)₂CH₂CH₃, —NHC(O)CH═CH₂ or —NH-cyclopropyl;

X is —C(Rₓ)═ or —N═;

wherein Rₓ is H, or Rₓ and Rₛ are taken together with the C atoms to which they are attached to form 5- to 6-membered heterocyclyl or 5- to 6-membered heteroaryl; alternatively 5- to 6-membered heteroaryl (alternatively pyrazinyl);

X₁ is —CH₂— or —N(R_{X1})—;

X₂ is —CH(R_{X2})— or —N(R_{X2})—;
wherein R_{X1} is —C(O)CH₂CH₃ or —C(O)CH═CH₂; R_{X2} is H, -Me, —OMe, —NHMe, —C(O)CH₂CH₃, —NHS(O)₂CH₂CH₃ or —NHC(O)CH₂CH₃;

[symbol] represents the point of attachment to L;

T is selected from bond, C₂₋₆ heteroalkylene, 4- to 12-membered heterocyclylene, or 5- to 6-membered heterocyclylene, wherein the 5- to 6-membered heterocyclylene is substituted with 5- to 6-membered heterocyclylene; or is alternatively the following groups:

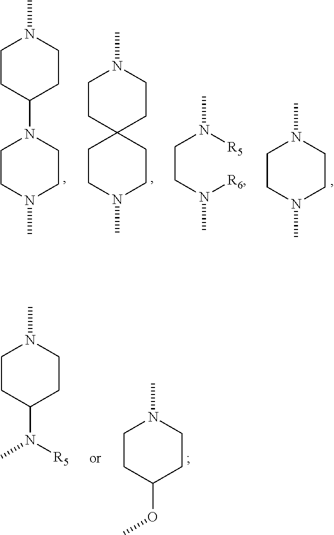

wherein

R₅ is -Me;

R₆ is -Me;

or R₅ and R₆ are connected to form —CH₂CH₂—;

[symbol] represents the point of attachment to parent core or L₂;

R₁ is selected from H, —Cl, —Br, —CH₃, —CF₃, cyclopropyl or —CH═CH₂;

or, when L is —NR—, R₁ and R are taken together with the atoms to which they are attached to form optionally substituted 5- to 6-membered heterocyclyl, wherein the substituent is selected from halogen, oxo, -iPr, -Et, or phenyl, wherein the phenyl is mono- or poly-substituted with halogen; R₁ and R are alternatively taken together to form the following groups: —C(O)N(R_{N})C(O)— or —C(CH₃)═C(R_{N})C(O)—;

wherein

R_{N} is selected from -iPr, -Et or

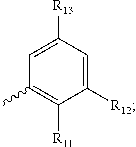

R₁₁ is —Cl, —Br;

R₁₂ is H or —F;

R₁₃ is H or —F;

[symbol] represents the point of attachment;

R₂ is H;

or R₁ and R₂ are taken together with the atoms to which they are attached to form 5- to 6-membered heterocyclyl or 5- to 6-membered heteroaryl; alternatively form pyrrolyl;

R₃ is selected from H or —OMe;

R₄ is selected from H, —F, -Me, —CF₃ or —NHC(O)CH═CH₂;

other groups are as defined above;

In an embodiment of the compound of formula (I) or (I-G), L₁ is selected from bond, —O—, —NH—, —CH₂—, —C(O)—, —CH₂CH₂—, —CH═CH—, —C≡C—, —OCH₂—, —CH₂O—, —NHCH₂—, —CH₂NH—, —C(O)CH₂—, —CH₂C(O)—, —OC(O)—, —C(O)O—, —NHC(O)— or —C(O)NH—;

E is independently selected from —CH₂CH₂CH₂—, —CH₂CH₂C(O)—, —CH₂C(O)CH₂—, —C(O)CH₂CH₂—, —C(O)CH═CH—, —C(O)C≡C—, —CH₂CH₂O—, —CH₂OCH₂—, —OCH₂CH₂—, —C(O)CH₂O—, —OCH₂C(O)—, —CH₂C(O)O—, —OC(O)CH₂—, —C(O)OCH₂—, —CH₂OC(O)—, —CH₂CH₂NH—, —CH₂NHCH₂—, —NHCH₂CH₂—, —C(O)CH₂NH—, —NHCH₂C(O)—, —CH₂C(O)NH—, —NHC(O)CH₂—, —C(O)NHCH₂—, —CH₂NHC(O)—,

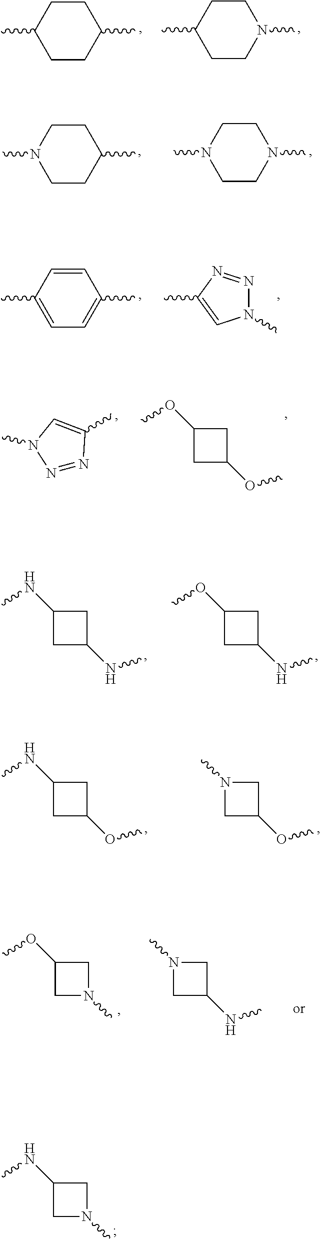

m is 0, 1, 2, 3, 4, 5, 6, 7, 8, 9 or 10;

L₂ is selected from bond, —CH₂—, —CH₂CH₂—, —OCH₂—, —NHCH₂—, —OC(O)—, —NHC(O)—, —CH₂C(O)— or —C(O)CH₂—;

wherein [symbol] represents the point of attachment to L₁ or L₂.

In a more specific embodiment, the present disclosure provides a compound of formula (I), or a pharmaceutically acceptable salt, enantiomer, diastereomer, racemate, solvate, hydrate, polymorph, prodrug, or isotopic variant thereof, or mixture thereof, wherein the compound of formula (I) has the structure of the following formulas:

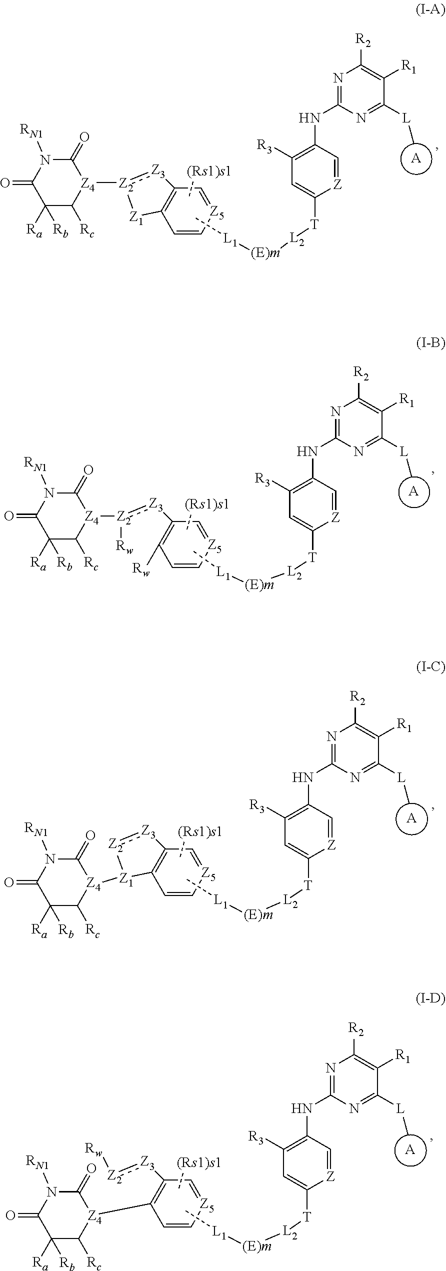

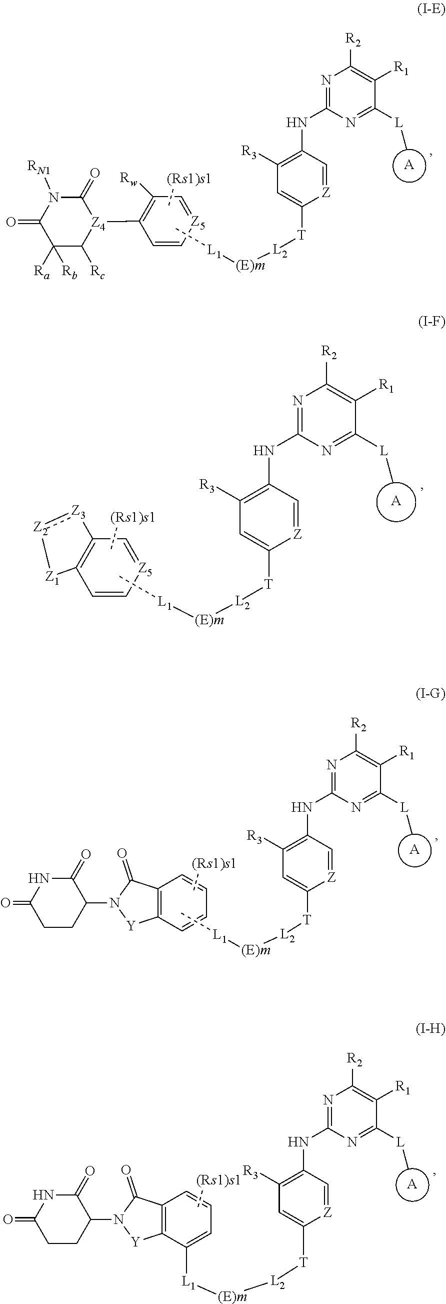

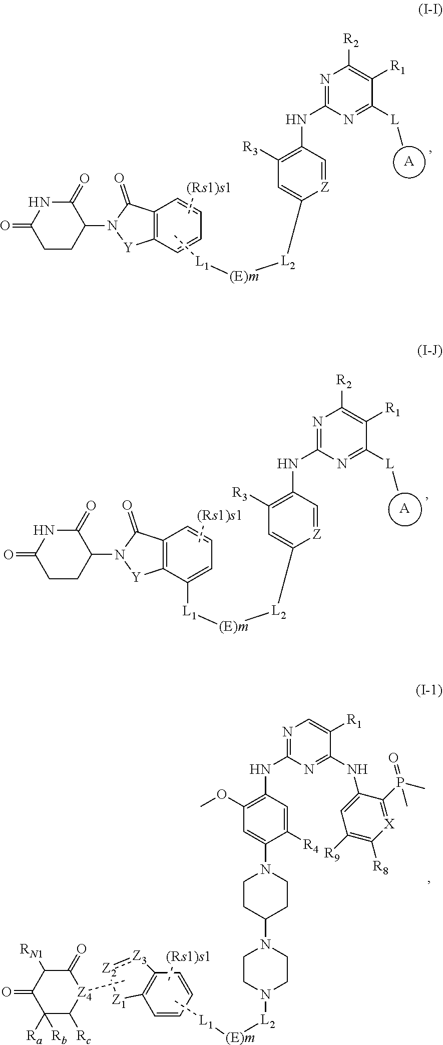

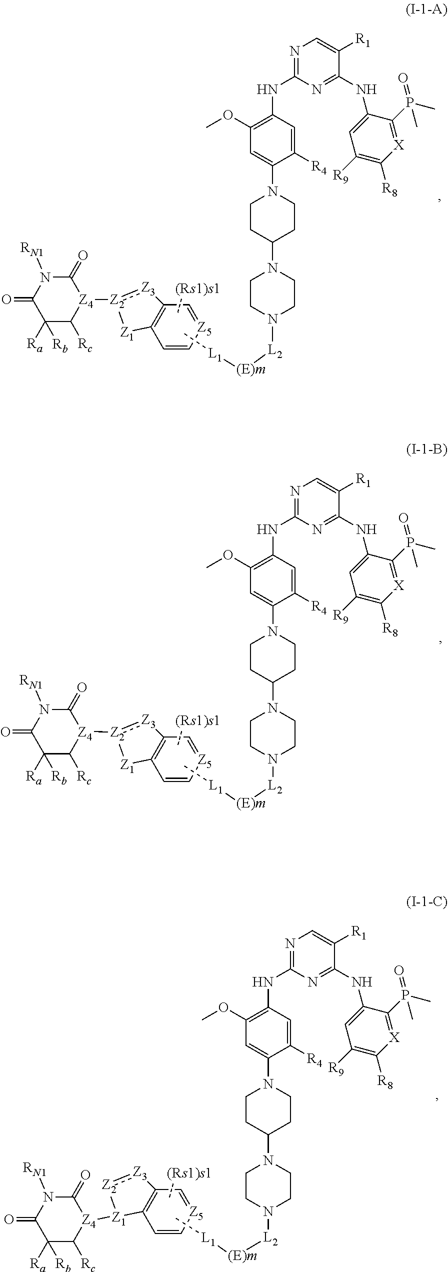

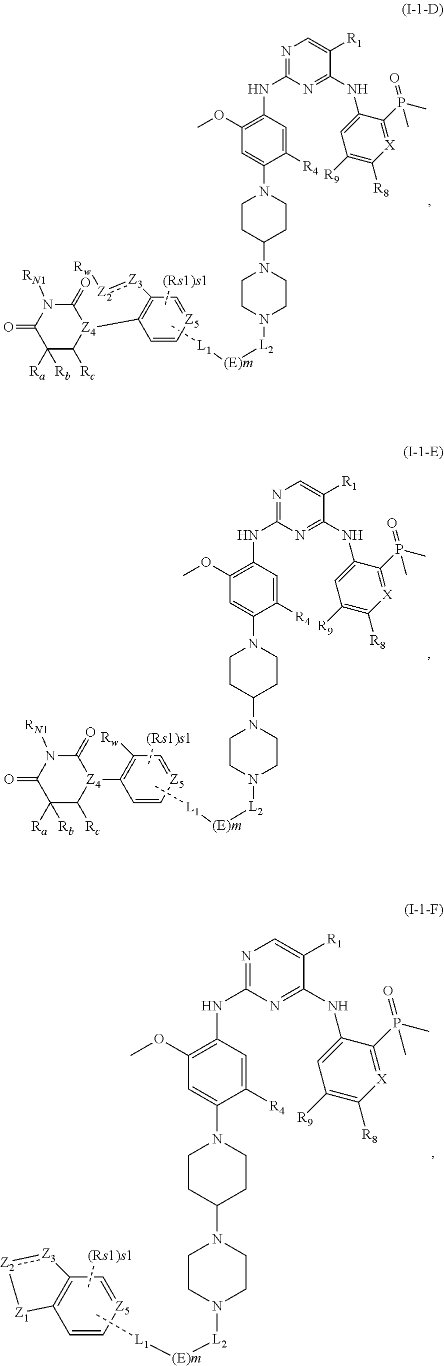

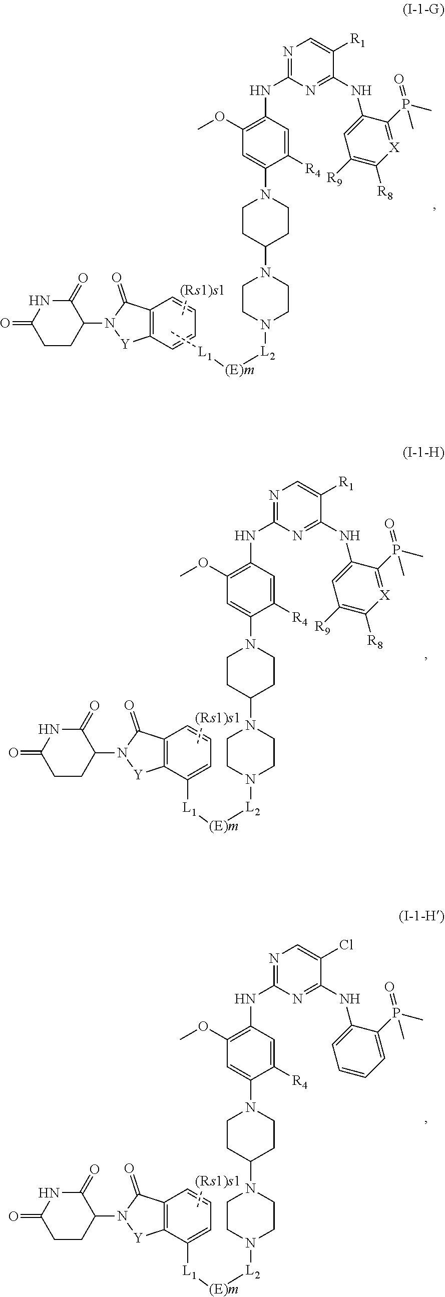

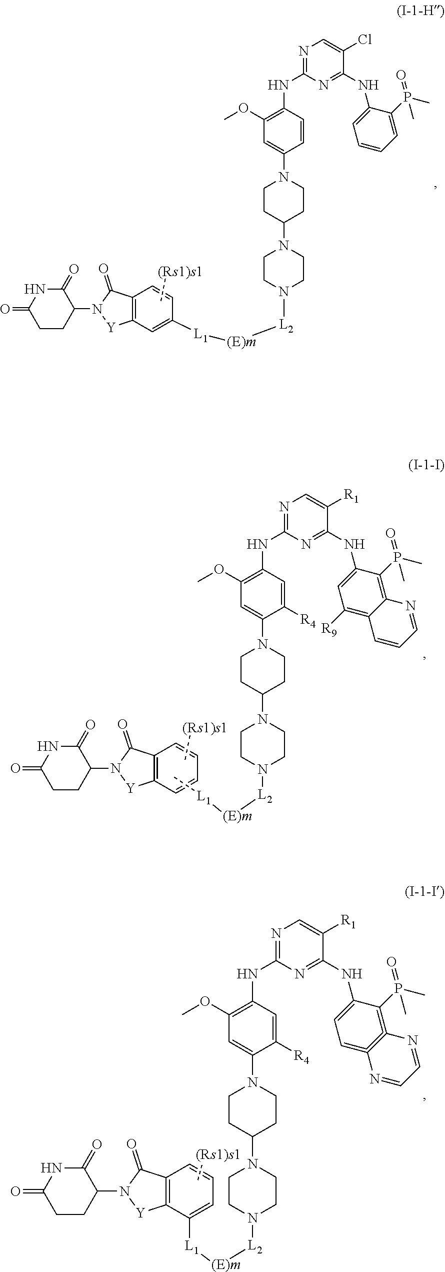

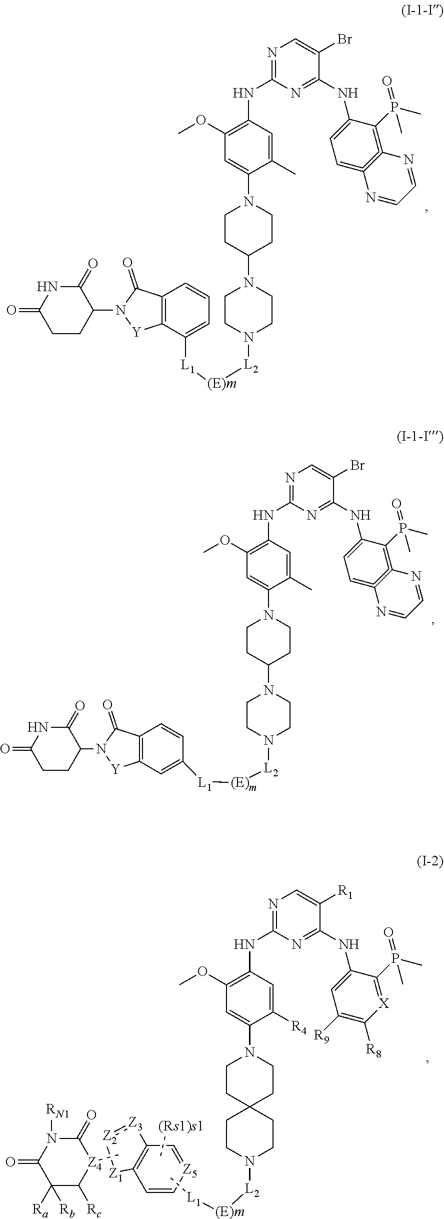

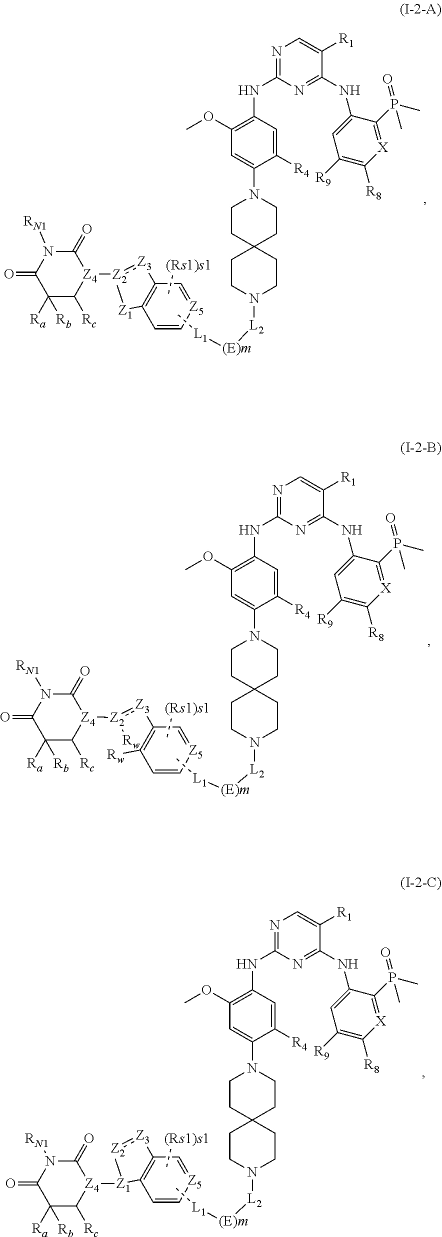

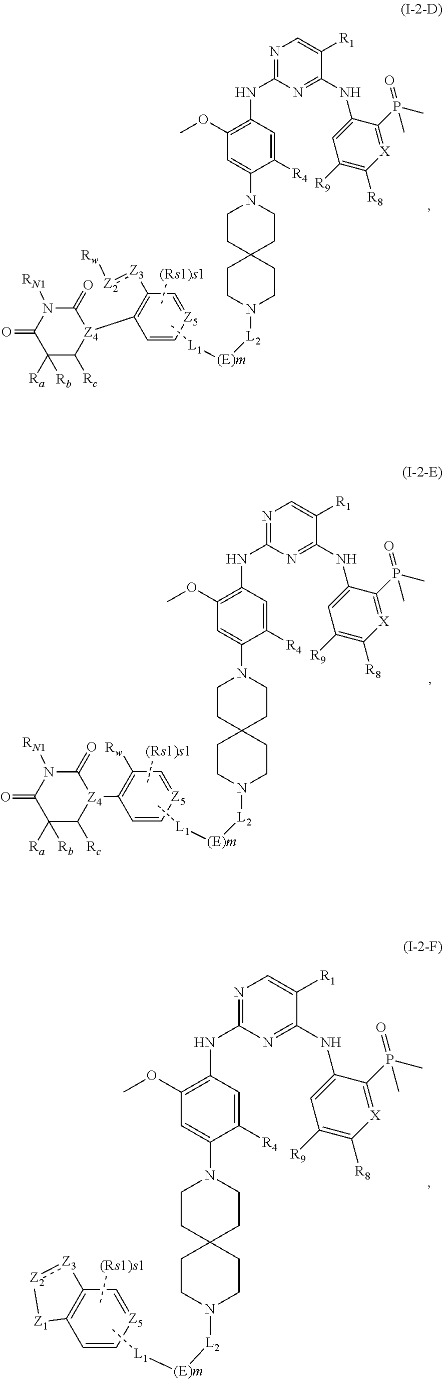

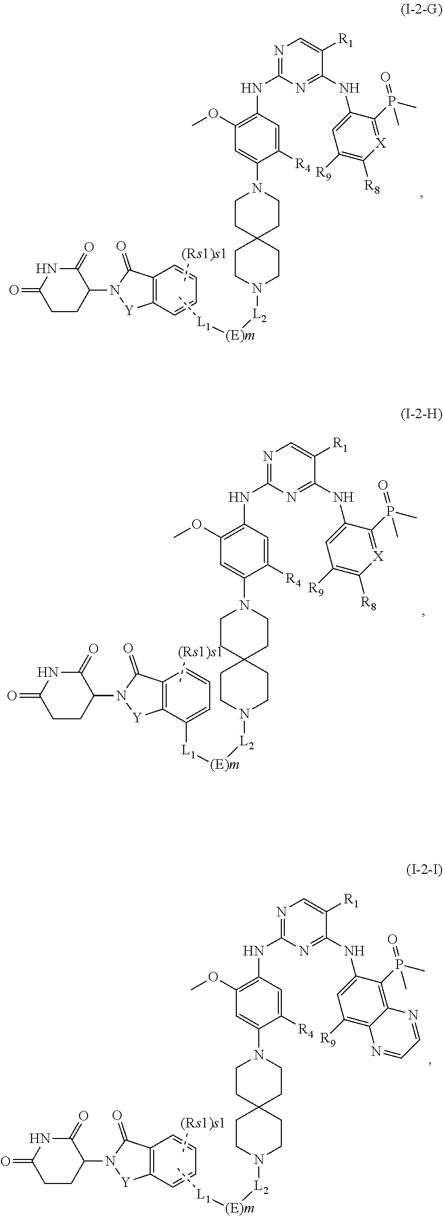

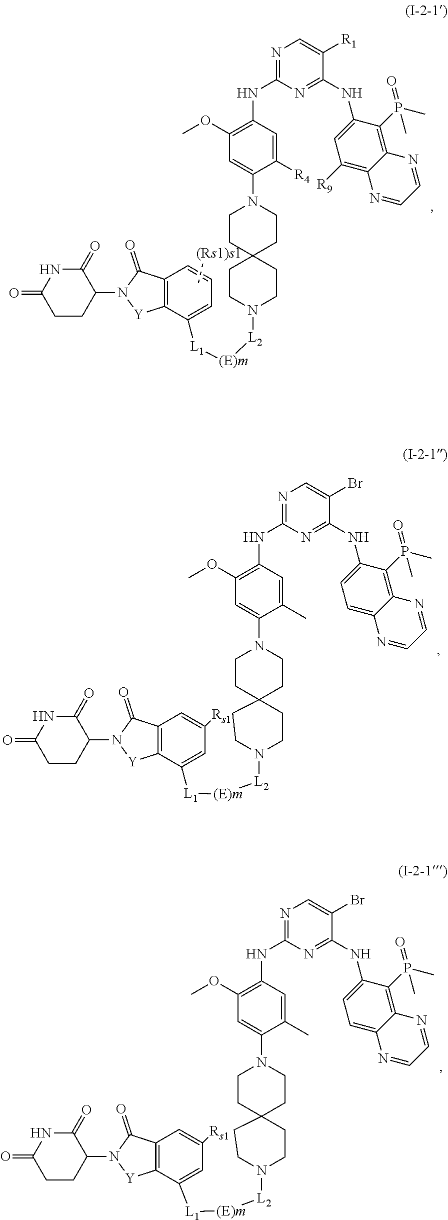

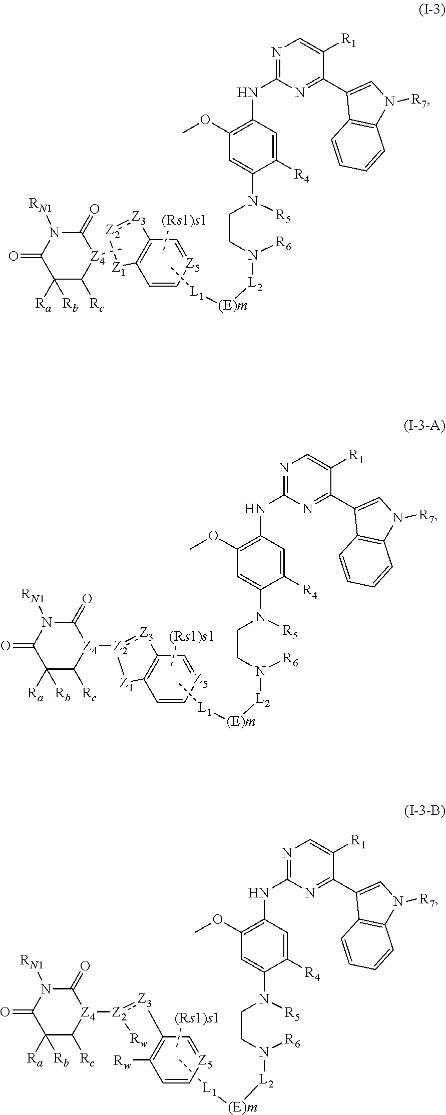

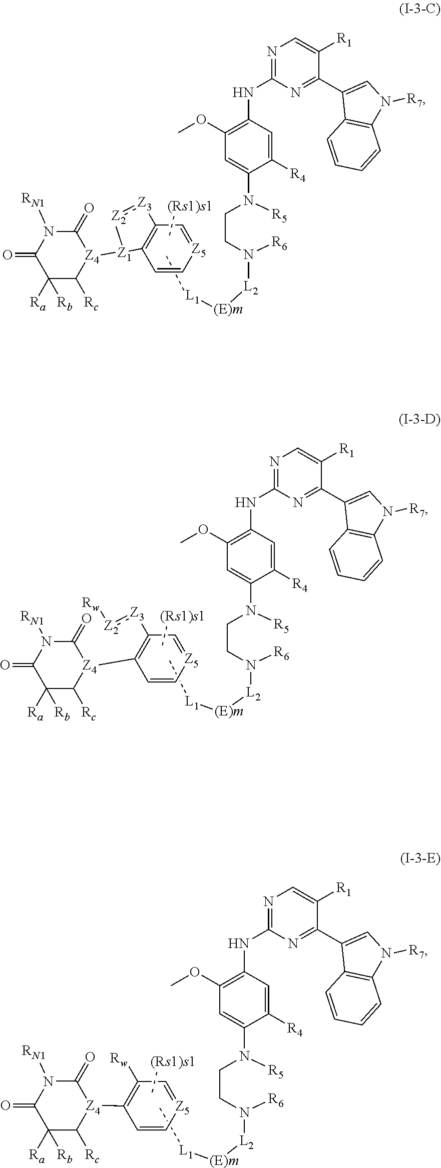

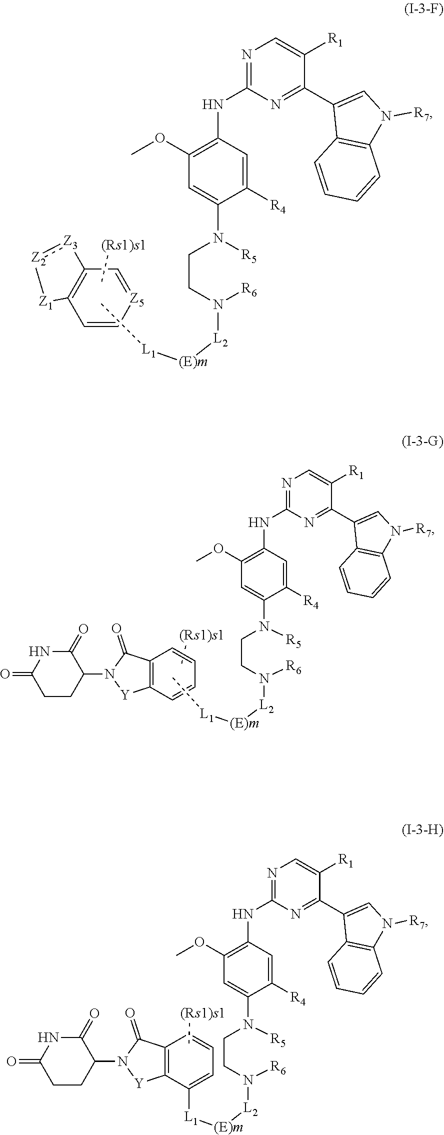

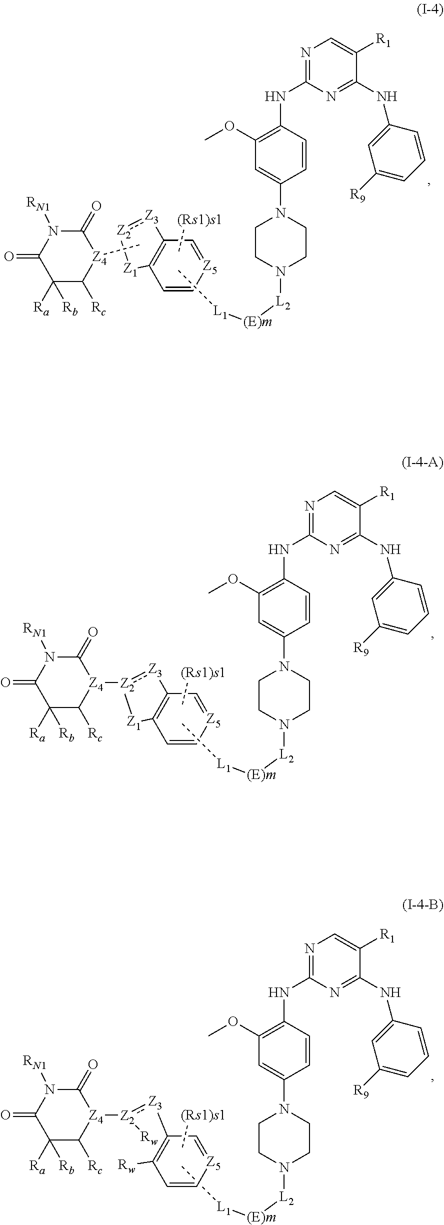

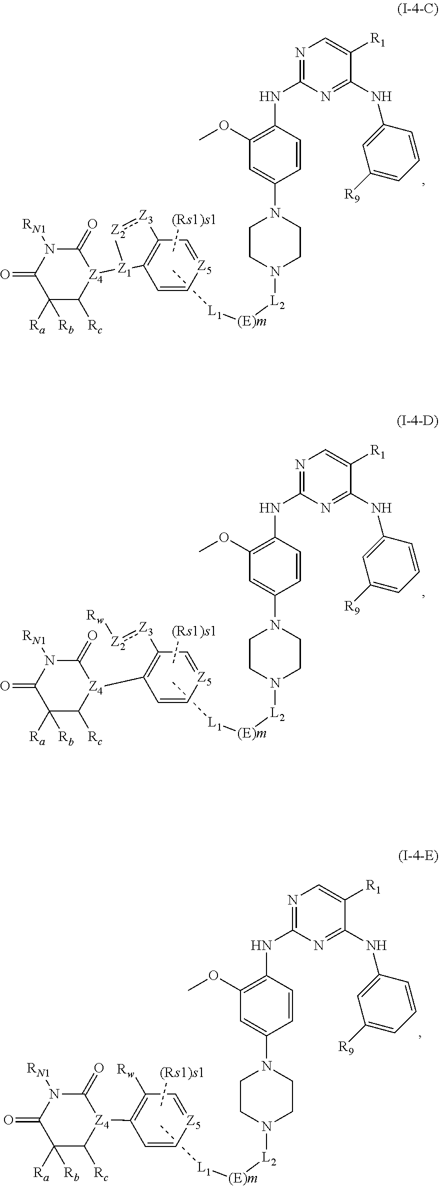

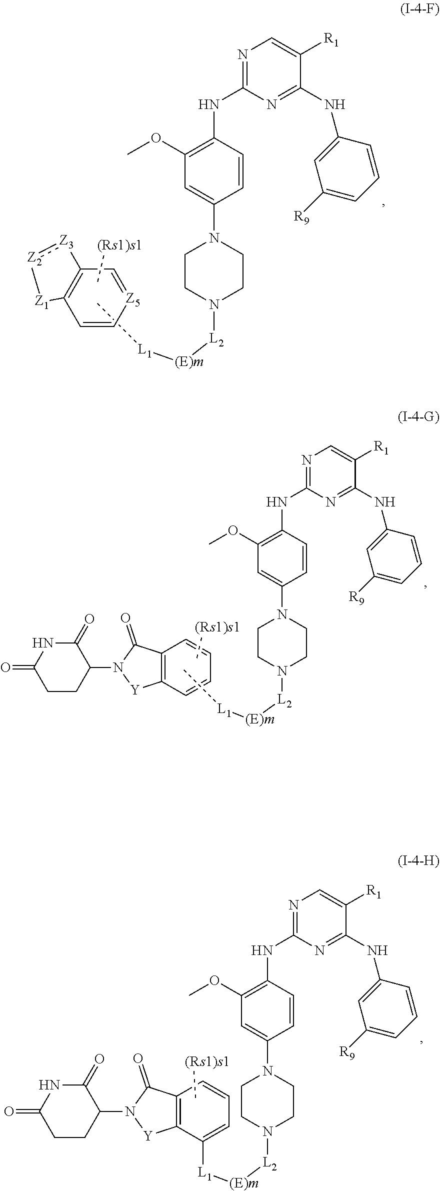

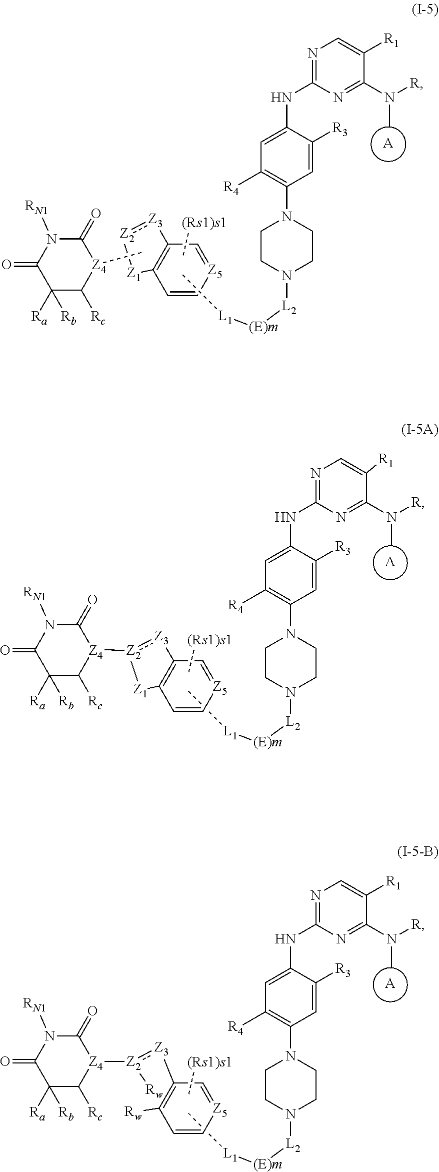

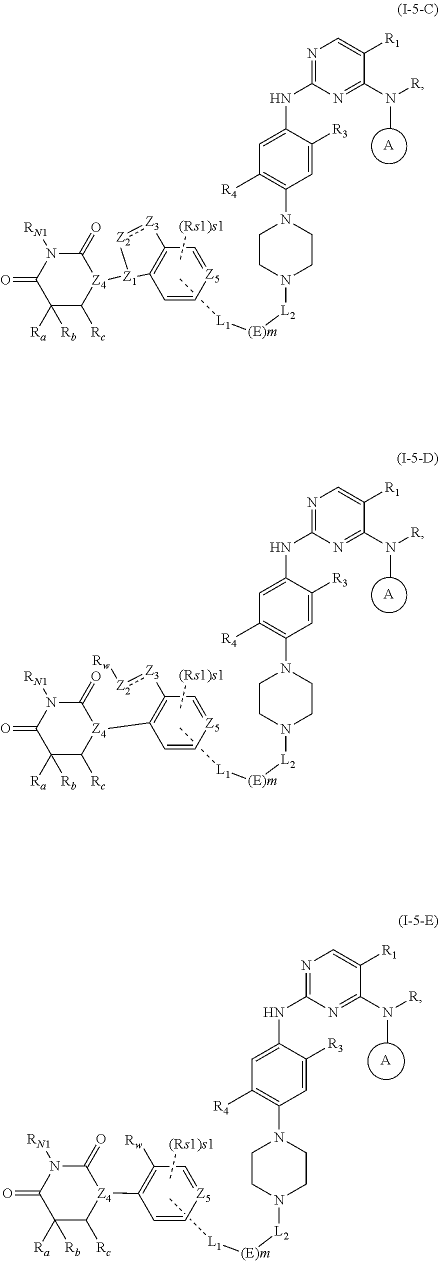

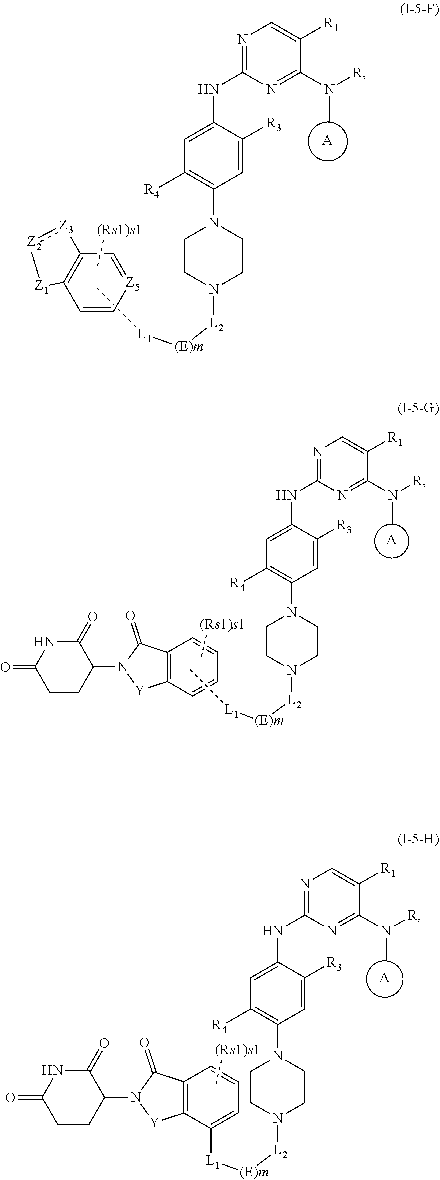

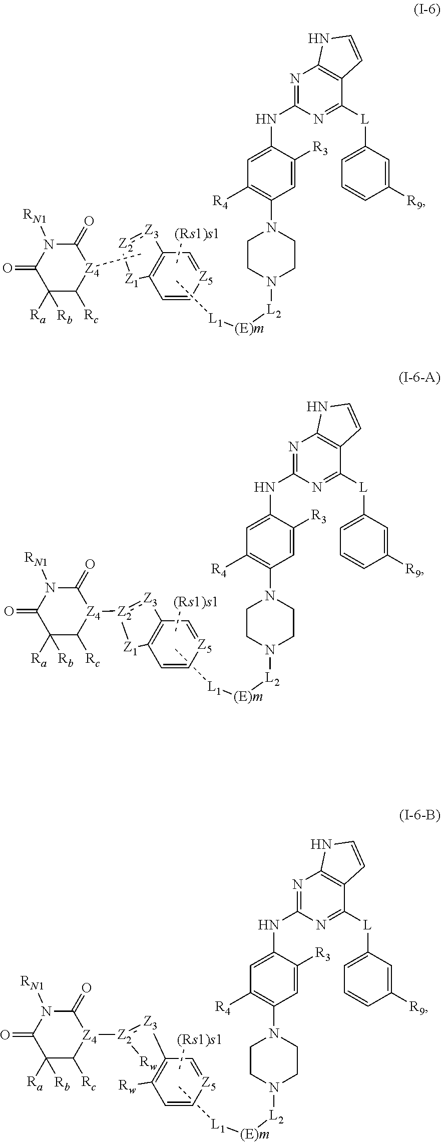

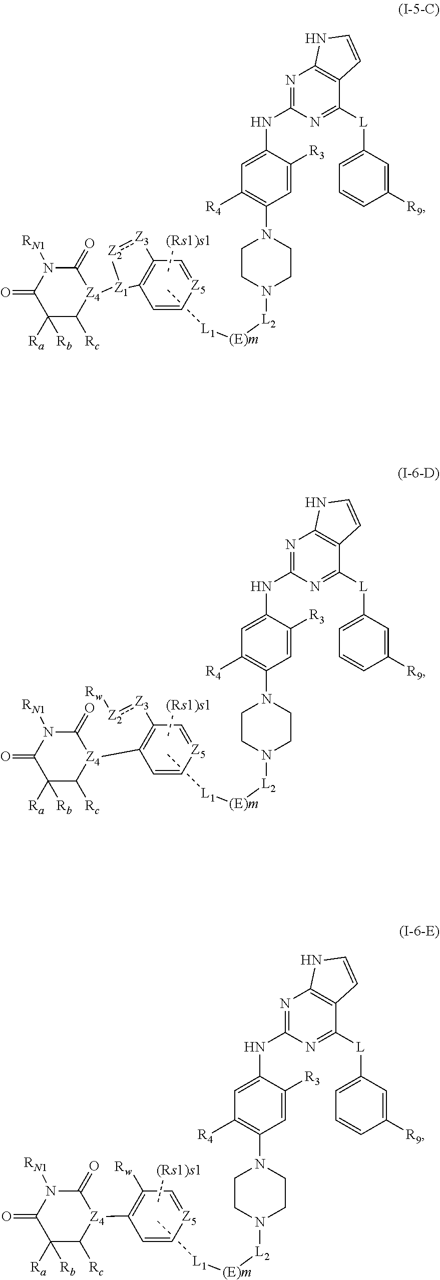

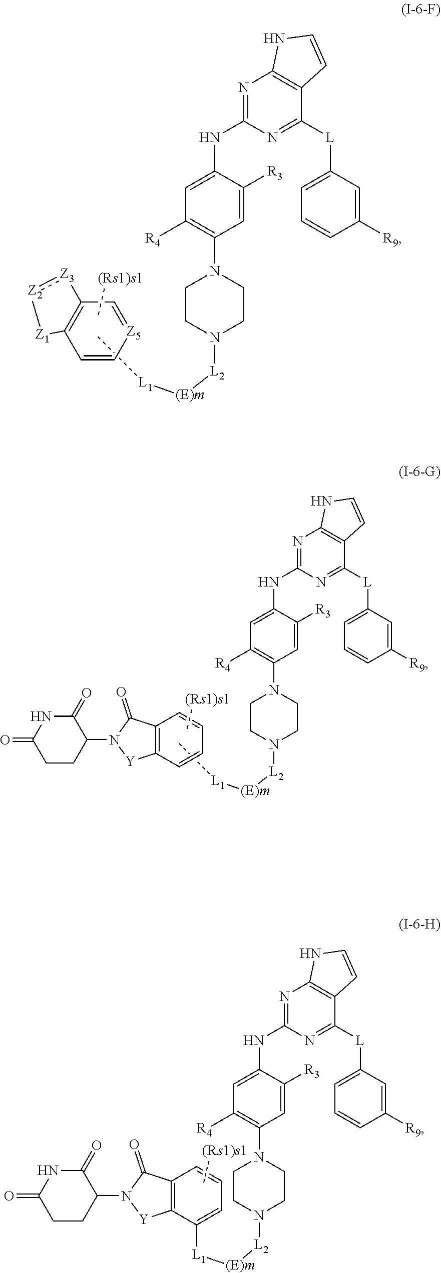

wherein each group is as defined in above.

In a more specific embodiment, the present disclosure provides a compound of formula (I-1), (I-1-A), (I-1-B), (I-1-C), (I-1-D), (I-1-E), (I-1-F), (I-1-G), (I-1-H) or (I-1-I), or a pharmaceutically acceptable salt, enantiomer, diastereomer, racemate, solvate, hydrate, polymorph, prodrug, or isotopic variant thereof, or mixture thereof, wherein

R₁ is selected from —Cl, —Br, —CF₃ or —CH═CH₂; alternatively, R₁ is selected from —C₁ or —Br;

R₄ is H or -Me;

R₈ is H, —NHMe, —NHC(O)CH₂CH₃ or —NH-cyclopropyl;

R₉ is H, —F, —Cl, —Br, -Me, —CF₃, —OMe, —OCF₃, —CN, —NHC(O)CH₂CH₃, —NHS(O)₂CH₂CH₃ or —NHC(O)CH═CH₂;

X is —C(Rₓ)═ or —N═;

Rₓ is H, or Rₓ and R₈ are taken together with the C atoms to which they are attached to form pyrazinyl;

and other groups are as defined in above.

In a more specific embodiment, the present disclosure provides a compound of formula (I-G), or a pharmaceutically acceptable salt, enantiomer, diastereomer, racemate, solvate, hydrate, polymorph, prodrug, or isotopic variant thereof, or mixture thereof:

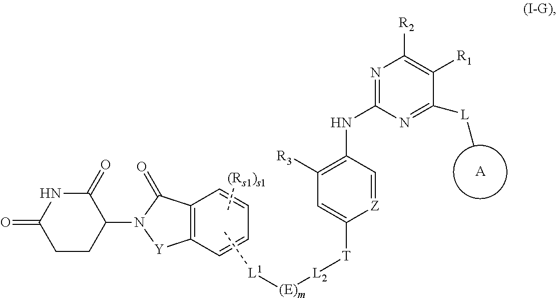

wherein

ring A is the following group:

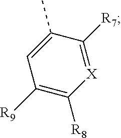

wherein

R₇ is —P(O)(C₁₋₆ alkyl)₂;

R₈ is H;

R₉ is selected from H, halogen, C₁₋₆ alkyl, —CN or C₁₋₆ haloalkyl;

X is —C(Rₓ)═;

wherein Rₓ is H, or Rₓ and Rₛ are taken together with the C atoms to which they are attached to form 5- to 6-membered heteroaryl; alternatively form pyrazinyl;

Y is C atom, which is optionally substituted with one or two R_{Z1}, wherein R_{Z1} is H, CN or halogen; or two R_{Z1} are taken together with Y to form C═O; alternatively, Y is CH₂ or C═O; alternatively, Y is CH₂;

L₁ is —CₐR^{#}R^{#}′—C_{b}R^{#}R^{#}′—;

L₂ is selected from bond, —CR^{#}R^{#}′— or —CₐR^{#}R^{#}′—C_{b}R^{#}R^{#}′—;

wherein one of CₐR^{#}R^{#}′ and C_{b}R^{#}R^{#}′ can be replaced by O, S(O)ₚ or NR^{#};

E is independently selected from: bond or —CₑR^{#}R^{#}′—C_{d}R^{#}R^{#}′—CₑR^{#}R^{#}′;

wherein one of C_{c}R^{#}R′, C_{d}R^{#}R^{#}′ or CₑR^{#}R^{#}′, or both of C_{c}R^{#}R′ and CₑR^{#}R^{#}′ can be replaced by O, S(O)ₚ or NR^{#};

p is 0, 1 or 2;

R^{#} is H, halogen, C₁₋₆ alkyl, C₁₋₆ haloalkyl, C₂₋₆ alkenyl or C₂₋₆ alkynyl;

R^{#}′ is H, halogen, C₁₋₆ alkyl, C₁₋₆ haloalkyl, C₂₋₆ alkenyl or C₂₋₆ alkynyl;

or, R^{#} and R^{#} on adjacent atoms can be taken together to form bond, R^{#}′ and R^{#}′ on adjacent atoms can be taken together to form bond;

or, R^{#} and R^{#}′ on the same or different atoms can be taken together to form ═O;

m is 0, 1, 2, 3, 4 or 5;

Rsi is selected from H, CN, halogen, OH, NH₂, C₂₋₆ alkenyl, C₂₋₆ alkynyl, —O—C₁₋₆ alkyl, —O—C₁₋₆ haloalkyl, —NH—C₁₋₆ alkyl, C₁₋₆ alkyl or C₁₋₆ haloalkyl;

s1 is 0, 1, 2 or 3;

L is —NR—; wherein R is H or C₁₋₆ alkyl;

Z is —C(R₄)═;

T is

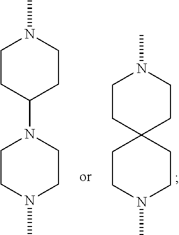

R₁ is selected from H, halogen, cyano, C₁₋₆ alkyl, C₁₋₆ haloalkyl, C₂₋₆ alkenyl or C₂₋₆ alkynyl;

R₂ is H, halogen, hydroxyl, amino, C₁₋₆ alkyl or C₁₋₆ haloalkyl;

R₃ is selected from H, —O—C₁₋₆ alkyl or —O—C₁₋₆ haloalkyl;

R₄ is selected from H, halogen, C₁₋₆ alkyl or C₁₋₆ haloalkyl;

if the above groups are H or H-containing groups, the one or more H atom(s) may be substituted with D atom(s).

In a more specific embodiment, the present disclosure provides a compound of formula (I-1-G), (I-1-H), (I-1-H′), (I-1-H″), or a pharmaceutically acceptable salt, enantiomer, diastereomer, racemate, solvate, hydrate, polymorph, prodrug, or isotopic variant thereof, or mixture thereof:

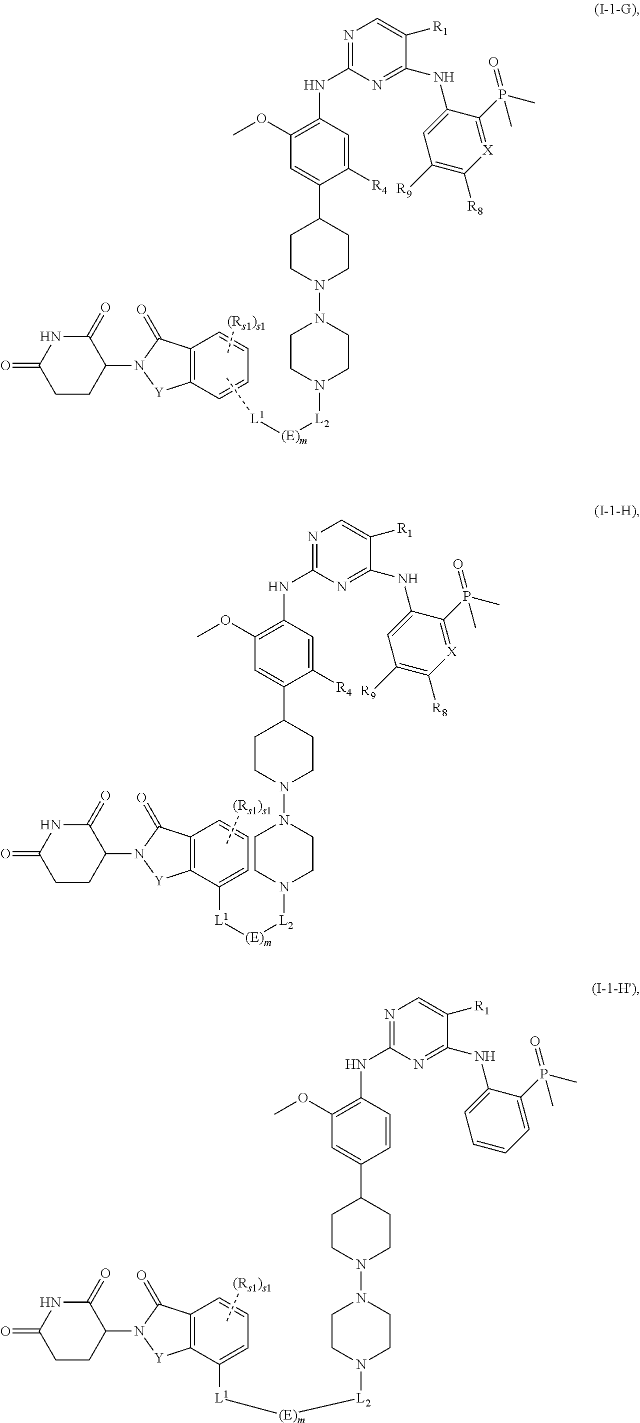

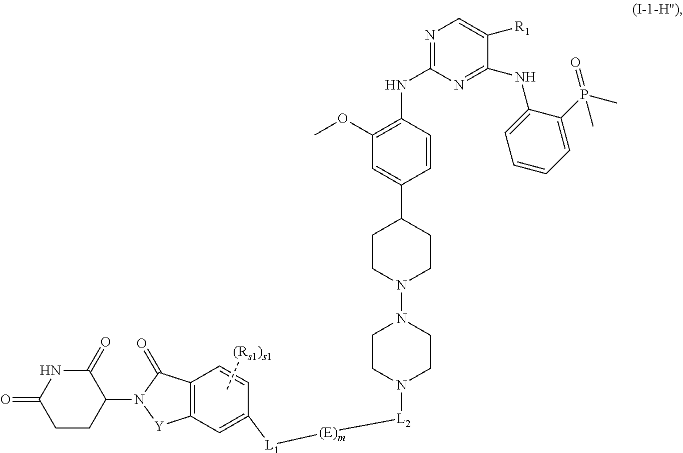

wherein

X is —CH═;

Y is C atom, which is optionally substituted with one or two R_{Z1}, wherein R_{Z1} is H, CN or halogen; or two R_{Z1} are taken together with Y to form C═O; alternatively, Y is CH₂ or C═O;

R₁ is selected from H, halogen, cyano, CH, alkyl, C₁₋₆ haloalkyl, C₂₋₆ alkenyl or C₂₋₆ alkynyl; alternatively, R₁ is H or halogen;

R₄ is selected from H, halogen, C₁₋₆ alkyl or C₁₋₆ haloalkyl; alternatively, R₄ is H;

R₈ is H;

R₉ is selected from H, halogen, C₁₋₆ alkyl, —CN or C₁₋₆ haloalkyl; alternatively, R₉ is H;

L₁ is selected from —CH₂CH₂—, —CH═CH—, —C═C—, —OCH₂—, —SCH₂—, —S(O)CH₂—, —S(O)₂CH₂—, —NHCH₂—, —N(Me)CH₂—, —C(O)CH₂—, —CH₂C(O)—, —OC(O)—, —SC(O)—, —NHC(O)— or —N(Me)C(O)—, and one or more H atom(s) in the above groups can be substituted with D atom(s); alternatively, L₁ is selected from —CH₂CH₂—, —CH═CH—, —C═C—, —OCH₂— or —NHCH₂—; alternatively, L₁ is selected from —CH₂CH₂—, —C≡C— or —OCH₂—;

L₂ is selected from bond, —CH₂— or —CH₂CH₂—, and one or more H atom(s) in the above groups can be substituted with D atom(s);

E is —CH₂CH₂CH₂—, —CH₂CH₂O—, —CH₂OCH₂—, —OCH₂CH₂—, —CH₂CH₂S—, —CH₂SCH₂— or —SCH₂CH₂—, and one or more H atom(s) in the above groups can be substituted with D atom(s); alternatively, E is —CH₂CH₂CH₂—;

m is 0, 1, 2 or 3; alternatively, the chain length of -L₁-(E)ₘ-L₂- is 4 to 14 bond lengths, still alternatively 5, 6, 7, 8, 9 or 10 bond lengths;

Rₛ₁ is selected from H, CN, halogen, OH, NH₂, C₂₋₆ alkenyl, C₂₋₆ alkynyl, —O—C₁₋₆ alkyl, —O—C₁₋₆ haloalkyl, —NH—C₁₋₆ alkyl, C₁₋₆ alkyl or C₁₋₆ haloalkyl; alternatively, Rₛ₁ is H, CN or halogen;

s1 is 0, 1 or 2.

In a more specific embodiment, the present disclosure provides a compound of formula (I-1-G), (I-1-H), (I-1-H′), (I-1-H″), or a pharmaceutically acceptable salt, enantiomer, diastereomer, racemate, solvate, hydrate, polymorph, prodrug, or isotopic variant thereof, or mixture thereof:

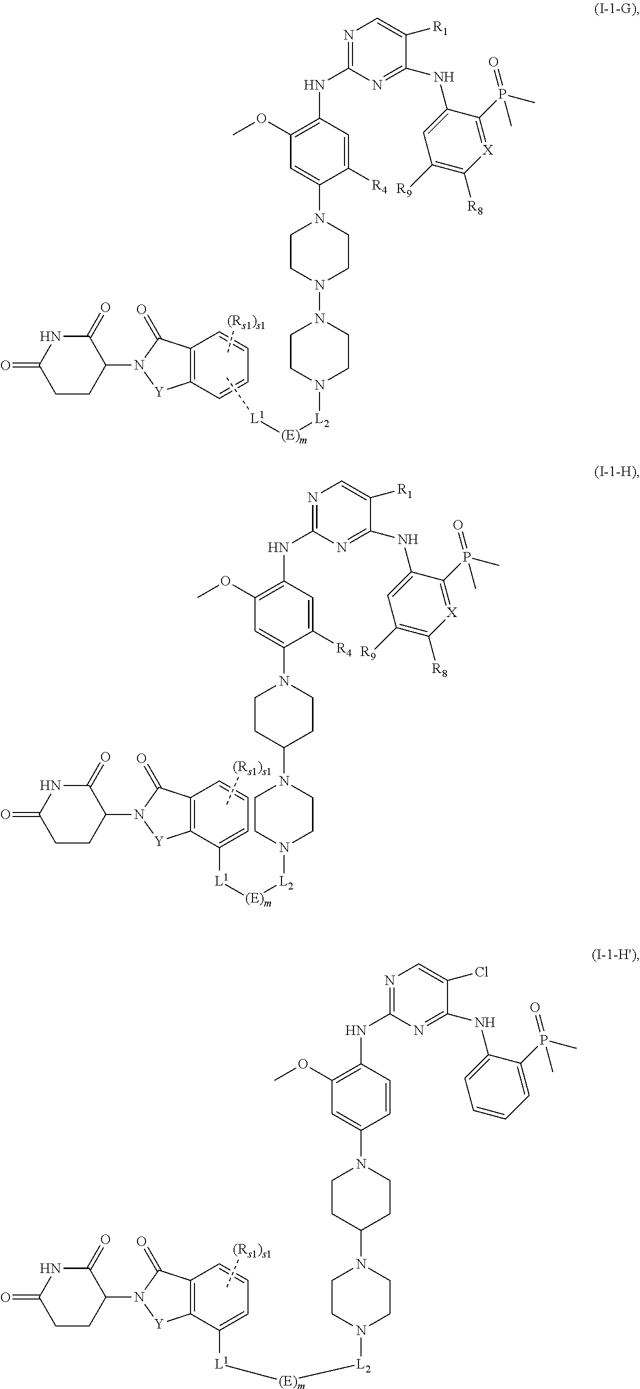

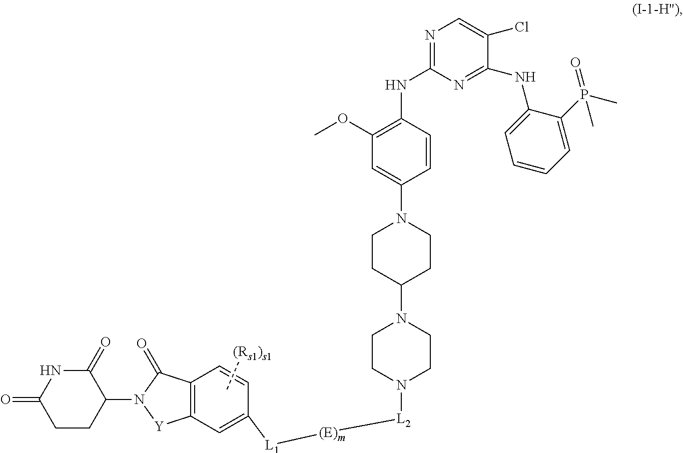

wherein

X is —CH═;

Y is C atom, which is optionally substituted with one or two R_{Z1}, wherein R_{Z1} is H, CN or halogen; or two R_{Z1} are taken together with Y to form C═O; alternatively, Y is CH₂;

R₁ is selected from H, halogen, cyano, C₁₋₆ alkyl, C₁₋₆ haloalkyl, C₂₋₆ alkenyl or C₂₋₆ alkynyl; alternatively, R₁ is halogen;

R₄ is selected from H, halogen, C₁₋₆ alkyl or C₁₋₆ haloalkyl; alternatively, R₄ is H;

R₈ is H;

R₉ is selected from H, halogen, C₁₋₆ alkyl, —CN or C₁₋₆ haloalkyl; alternatively, R₉ is H;

L₁ is selected from —CH₂CH₂—, —CH═CH—, —C≡C—, —OCH₂—, —SCH₂—, —S(O)CH₂—, —S(O)₂CH₂—, —NHCH₂—, —N(Me)CH₂—, —C(O)CH₂—, —CH₂C(O)—, —OC(O)—, —SC(O)—, —NHC(O)— or —N(Me)C(O)—, and one or more H atom(s) in the above groups can be substituted with D atom(s); alternatively, L₁ is selected from —CH₂CH₂—, —CH═CH—, —C≡C—, —OCH₂— or —NHCH₂—; alternatively, L₁ is selected from —CH₂CH₂—, —C≡C—, —OCH₂— or —NHCH₂—;

L₂ is selected from bond, —CH₂— or —CH₂CH₂—, and one or more H atom(s) in the above groups can be substituted with D atom(s);

E is —CH₂CH₂CH₂—, —CH₂CH₂O—, —CH₂OCH₂—, —OCH₂CH₂—, —CH₂CH₂S—, —CH₂SCH₂— or —SCH₂CH₂—, and one or more H atom(s) in the above groups can be substituted with D atom(s); alternatively, E is —CH₂CH₂CH₂—;

m is 0, 1, 2 or 3; alternatively, the chain length of -L₁-(E)ₘ-L₂- is 4 to 14 bond lengths, still alternatively 5, 6, 7, 8, 9 or 10 bond lengths;

Rₛ₁ is selected from H, CN, halogen, OH, NH₂, C₂₋₆ alkenyl, C₂₋₆ alkynyl, —O—C₁₋₆ alkyl, —O—C₁₋₆ haloalkyl, —NH—C₁₋₆ alkyl, C₁₋₆ alkyl or C₁₋₆ haloalkyl; alternatively, Rₛ₁ is H or halogen;

s1 is 0, 1 or 2.

In a more specific embodiment, the present disclosure provides a compound of formula (I-1-G), (I-1-H), (I-1-H′), (I-1-H″), or a pharmaceutically acceptable salt, enantiomer, diastereomer, racemate, solvate, hydrate, polymorph, prodrug, or isotopic variant thereof, or mixture thereof:

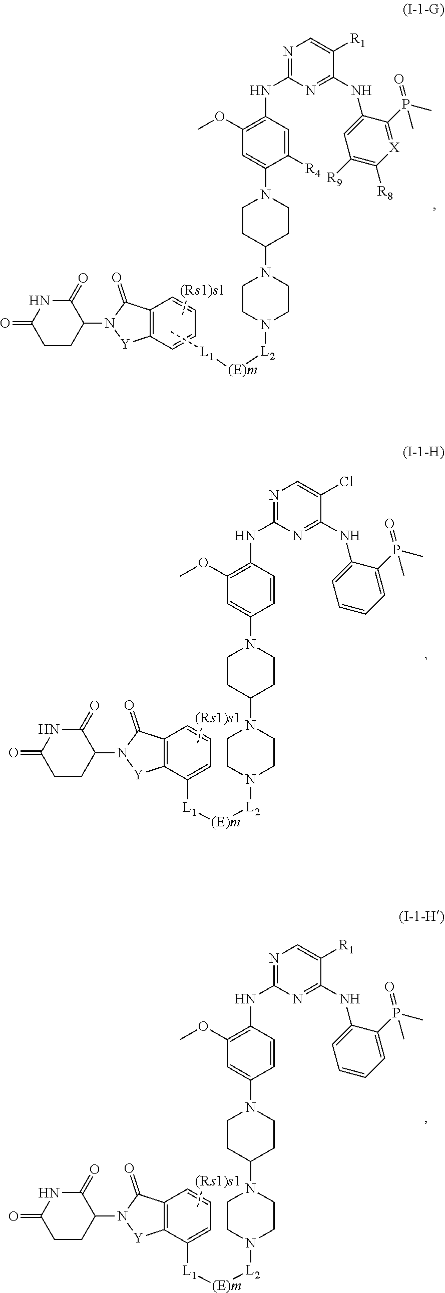

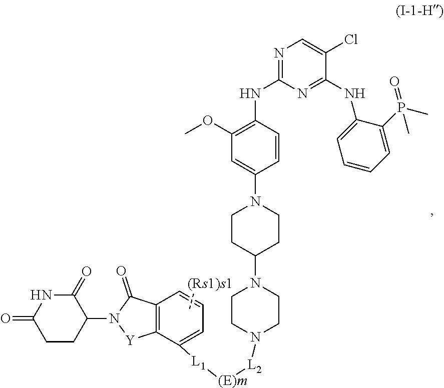

wherein

X is —CH═;

Y is C atom, which is optionally substituted with one or two R_{Z1}, wherein R_{Z1} is H, CN or halogen; or two R_{Z1} are taken together with Y to form C═O; alternatively, Y is CH₂ or C═O;

R₁ is selected from H, halogen, cyano, C₁₋₆ alkyl, C₁₋₆ haloalkyl, C₂₋₆ alkenyl or C₂₋₆ alkynyl; alternatively, R₁ is halogen;

R₄ is selected from H, halogen, C₁₋₆ alkyl or C₁₋₆ haloalkyl; alternatively, R₄ is H;

R₈ is H;

R₉ is selected from H, halogen, C₁₋₆ alkyl, —CN or C₁₋₆ haloalkyl; alternatively, R₉ is H;

L₁ is selected from —CH₂CH₂—, —CH═CH—, —C≡C—, —OCH₂—, —SCH₂—, —S(O)CH₂—, —S(O)₂CH₂—, —NHCH₂—, —N(Me)CH₂—, —C(O)CH₂—, —CH₂C(O)—, —OC(O)—, —SC(O)—, —NHC(O)— or —N(Me)C(O)—, and one or more H atom(s) in the above groups can be substituted with D atom(s); alternatively, L₁ is selected from —CH₂CH₂—, —CH═CH—, —C≡C—, —OCH₂— or —NHCH₂—; alternatively, L₁ is selected from —C≡C—, —OCH₂— or —NHCH₂—;

L₂ is selected from bond, —CH₂— or —CH₂CH₂—, and one or more H atom(s) in the above groups can be substituted with D atom(s);

E is —CH₂CH₂CH₂—, —CH₂CH₂O—, —CH₂OCH₂—, —OCH₂CH₂—, —CH₂CH₂S—, —CH₂SCH₂— or —SCH₂CH₂—, and one or more H atom(s) in the above groups can be substituted with D atom(s); alternatively, E is —CH₂CH₂CH₂—;

m is 1, 2 or 3; alternatively, the chain length of -L₁-(E)ₘ-L₂- is less than 14 bond lengths; alternatively, the chain length is 5-10 bond lengths, alternatively 5, 6, 7, 8, 9 or 10 bond lengths;

Rₛ₁ is selected from H, CN, halogen, OH, NH₂, C₂₋₆ alkenyl, C₂₋₆ alkynyl, —O—C₁₋₆ alkyl, —O—C₁₋₆ haloalkyl, —NH—C₁₋₆ alkyl, C₁₋₆ alkyl or C₁₋₆ haloalkyl; alternatively, Rₛ₁ is H or halogen;

s1 is 0, 1 or 2.

In a more specific embodiment, the present disclosure provides a compound of formula (I-1-I), (I-1-I′), (I-1-I″), (I-1-I″), or a pharmaceutically acceptable salt, enantiomer, diastereomer, racemate, solvate, hydrate, polymorph, prodrug, or isotopic variant thereof, or mixture thereof:

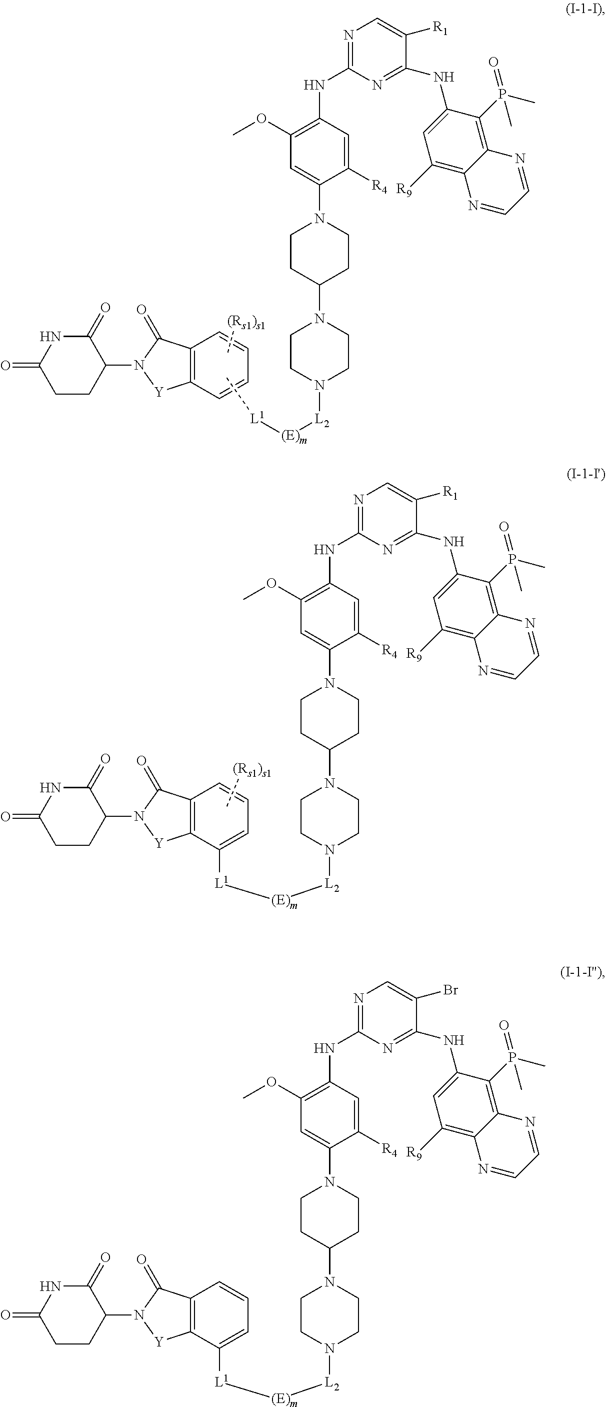

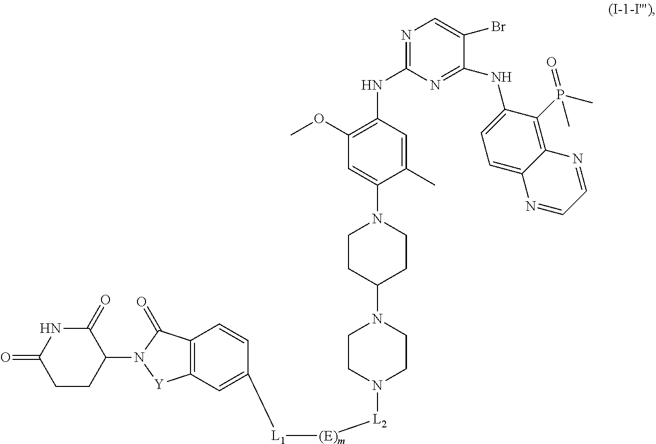

wherein

Y is C atom, which is optionally substituted with one or two R_{Z1}, wherein R_{Z1} is H, CN or halogen; or two R_{Z1} are taken together with Y to form C═O; alternatively, Y is CH₂ or C═O;

R₁ is selected from H, halogen, cyano, C₁₋₆ alkyl, C₁₋₆ haloalkyl, C₂₋₆ alkenyl or C₂₋₆ alkynyl; alternatively, R₁ is halogen;

R^{#} is selected from H, halogen, C₁₋₆ alkyl or C₁₋₆ haloalkyl; alternatively, R^{#} is selected from C₁₋₆ alkyl or C₁₋₆ haloalkyl;

R⁹ is selected from H, halogen, C₁₋₆ alkyl, —CN or C₁₋₆ haloalkyl; alternatively, R⁹ is H;

L₁ is selected from —CH₂CH₂—, —CH═CH—, —C≡C—, —OCH₂—, —SCH2-, —S(O)CH₂—, —S(O)₂CH₂—, —NHCH₂—, —N(Me)CH₂—, —C(O)CH₂—, —CH₂C(O)—, —OC(O)—, —SC(O)—, —NHC(O)— or —N(Me)C(O)—; alternatively, L₁ is selected from —CH₂CH₂—, —CH═CH—, —C≡C— or —OCH₂—; alternatively, L₁ is selected from —CH₂CH₂—, —C≡C— or —OCH₂—;

L₂ is selected from bond, —CH₂— or —CH₂CH₂—;

E is —CH₂CH₂CH₂—, —CH₂CH₂O—, —CH₂OCH₂—, —OCH₂CH₂—, —CH₂CH₂S—, —CH₂SCH₂— or —SCH₂CH₂—; alternatively, E is —CH₂CH₂CH₂—;

m is 1 or 2; alternatively, the chain length of -L₁-(E)ₘ-L₂- is 4 to 14 bond lengths, still alternatively 5, 6, 7, 8, 9 or 10 bond lengths;

Rₛ₁ is selected from H, CN, halogen, OH, NH₂, C₂₋₆ alkenyl, C₂₋₆ alkynyl, —O—C₁₋₆ alkyl, —O—C₁₋₆ haloalkyl, —NH—C₁₋₆ alkyl, C₁₋₆ alkyl or C₁₋₆ haloalkyl; alternatively, Rₛ₁ is H, halogen or CN;

s1 is 0, 1 or 2.

In a more specific embodiment, the present disclosure provides a compound of formula (I-1-I), (I-1-I′), (I-1-I″), (I-1-I′), or a pharmaceutically acceptable salt, enantiomer, diastereomer, racemate, solvate, hydrate, polymorph, prodrug, or isotopic variant thereof, or mixture thereof:

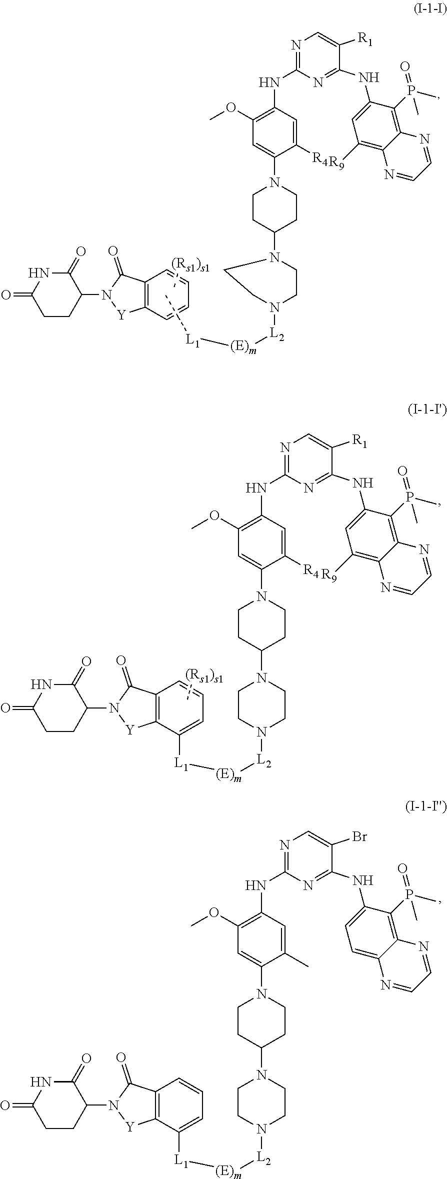

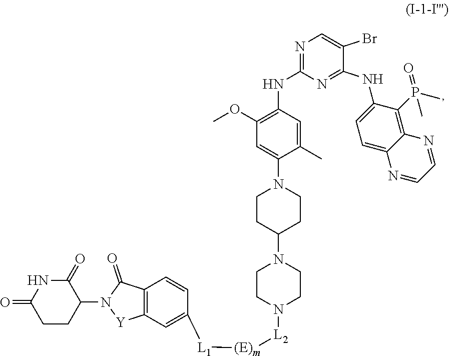

wherein

Y is C atom, which is optionally substituted with one or two R_{Z1}, wherein R_{Z1} is H, CN or halogen; or two R_{Z1} are taken together with Y to form C═O; alternatively, Y is CH₂;

R₁ is selected from H, halogen, cyano, C₁₋₆ alkyl, C₁₋₆ haloalkyl, C₂₋₆ alkenyl or C₂₋₆ alkynyl; alternatively, R₁ is halogen;

R^{#} is selected from H, halogen, C₁₋₆ alkyl or C₁₋₆ haloalkyl; alternatively, R^{#} is selected from C₁₋₆ alkyl or C₁₋₆ haloalkyl;

R₉ is selected from H, halogen, C₁₋₆ alkyl, —CN or C₁₋₆ haloalkyl; alternatively, R₉ is H;

L₁ is selected from —CH₂CH₂—, —CH═CH—, —C≡C—, —OCH₂—, —SCH₂—, —S(O)CH₂—, —S(O)₂CH₂—, —NHCH₂—, —N(Me)CH₂—, —C(O)CH₂—, —CH₂C(O)—, —OC(O)—, —SC(O)—, —NHC(O)— or —N(Me)C(O)—; alternatively, L₁ is selected from —CH₂CH₂—, —CH═CH—, —C≡C— or —OCH₂—; alternatively, L₁ is selected from —C≡C— or —OCH₂—;

L₂ is selected from bond, —CH₂— or —CH₂CH₂—;

E is —CH₂CH₂CH₂—;

m is 1 or 2; alternatively, the chain length of -L₁-(E)ₘ-L₂- is less than 14 bond lengths; still alternatively 5, 6, 7, 8, 9 or 10 bond lengths;

Rₛ₁ is selected from H, CN, halogen, OH, NH₂, C₂₋₆ alkenyl, C₂₋₆ alkynyl, —O—C₁₋₆ alkyl, —O—C₁₋₆ haloalkyl, —NH—C₁₋₆ alkyl, C₁₋₆ alkyl or C₁₋₆ haloalkyl; alternatively, Rₛ₁ is H or halogen;

s1 is 0, 1 or 2.

In a more specific embodiment, the present disclosure provides a compound of formula (I-2-I), (I-2-I′), (I-2-I″), (I-2-I″′), or a pharmaceutically acceptable salt, enantiomer, diastereomer, racemate, solvate, hydrate, polymorph, prodrug, or isotopic variant thereof, or mixture thereof:

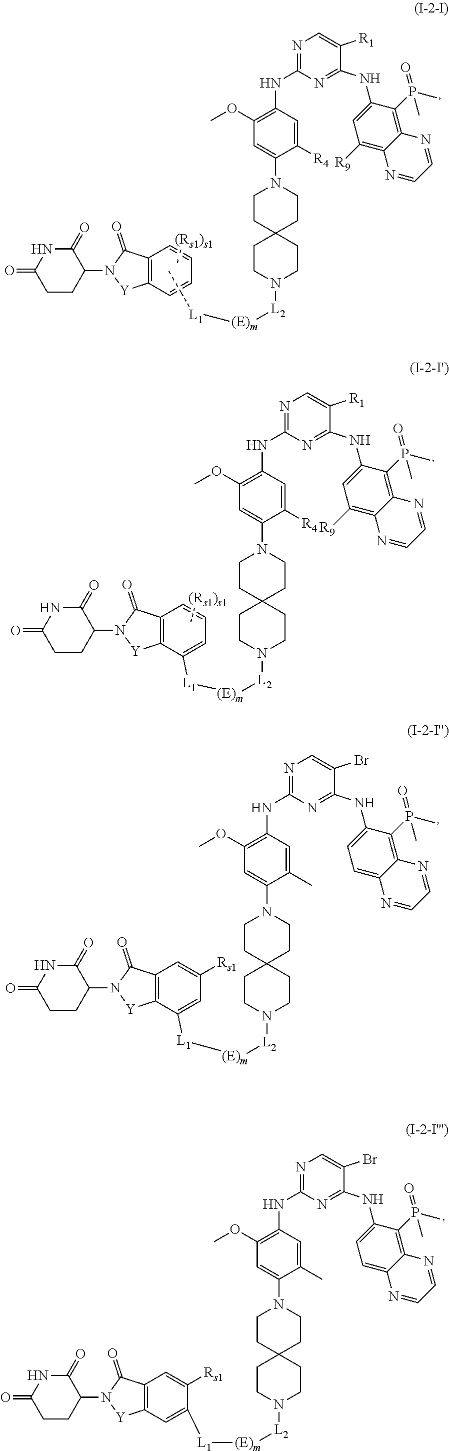

wherein

Y is C atom, which is optionally substituted with one or two R_{Z1}, wherein R_{Z1} is H, CN or halogen; or two R_{Z1} are taken together with Y to form C═O; alternatively, Y is CH₂;

R₁ is selected from H, halogen, cyano, C₁₋₆ alkyl, C₁₋₆ haloalkyl, C₂₋₆ alkenyl or C₂₋₆ alkynyl; alternatively, R₁ is halogen;

R₄ is selected from H, halogen, C₁₋₆ alkyl or C₁₋₆ haloalkyl; alternatively, R₄ is selected from C₁₋₆ alkyl or C₁₋₆ haloalkyl;

R₉ is selected from H, halogen, C₁₋₆ alkyl, —CN or C₁₋₆ haloalkyl; alternatively, R₉ is H;

L₁ is selected from —CH₂CH₂—, —CH═CH—, —C≡C—, —OCH₂—, —SCH₂—, —S(O)CH₂—, —S(O)₂CH₂—, —NHCH₂—, —N(Me)CH₂—, —C(O)CH₂—, —CH₂C(O)—, —OC(O)—, —SC(O)—, —NHC(O)— or —N(Me)C(O)—; alternatively, L₁ is selected from —CH₂CH₂—, —CH═CH—, —C≡C— or —OCH₂—; alternatively, L₁ is selected from —C≡C— or —OCH₂—;

L₂ is selected from bond, —CH₂— or —CH₂CH₂—;

E is —CH₂CH₂CH₂—, —CH₂CH₂O—, —CH₂OCH₂—, —OCH₂CH₂—, —CH₂CH₂S—, —CH₂SCH₂— or —SCH₂CH₂—; alternatively, E is —CH₂CH₂CH₂—;

m is 1 or 2; alternatively, the chain length of -L₁-(E)ₘ-L₂- is less than 14 bond lengths; alternatively 5, 6, 7, 8, 9 or 10 bond lengths;

Rₛ₁ is selected from H, CN, halogen, C₁₋₆ alkyl or C₁₋₆ haloalkyl; alternatively, Rₛ₁ is H or halogen;

s1 is 0, 1 or 2.

In a more specific embodiment, the present disclosure provides a compound of formula (I-2), (I-2-A), (I-2-B), (I-2-C), (I-2-D), (I-2-E), (I-2-F), (I-2-G), (I-2-H) or (I-2-I), or a pharmaceutically acceptable salt, enantiomer, diastereomer, racemate, solvate, hydrate, polymorph, prodrug, or isotopic variant thereof, or mixture thereof, wherein

R₁ is —Cl, —Br or —CH═CH₂;

R₄ is H or -Me;

R₈ is H, —NHMe, —NHC(O)CH₂CH₃ or —NH-cyclopropyl;

R₉ is H, —F, —Cl, —Br, -Me, —CF₃, —OMe, —OCF₃, —CN, —NHC(O)CH₂CH₃, —NHS(O)₂CH₂CH₃ or —NHC(O)CH═CH₂;

X is —C(Rₓ)═;

Rₓ is H, or Rₓ and R₈ are taken together with the C atoms to which they are attached to form pyrazinyl;

and other groups are as defined in above.

In a more specific embodiment, the present disclosure provides a compound of formula (I-3), (I-3-A), (I-3-B), (I-3-C), (I-3-D), (I-3-E), (I-3-F), (I-3-G) or (I-3-H), or a pharmaceutically acceptable salt, enantiomer, diastereomer, racemate, solvate, hydrate, polymorph, prodrug, or isotopic variant thereof, or mixture thereof, wherein

R₁ is H, —Cl or —CH═CH₂;

R₄ is —NHC(O)CH═CH₂;

R₅ is -Me;

R₆ is -Me;

or R₅ and R₆ are connected to form —CH₂CH₂—;

R₇ is -Me or cyclopropyl;

and other groups are as defined in above.

In a more specific embodiment, the present disclosure provides a compound of formula (I-4), (I-4-A), (I-4-B), (I-4-C), (I-4-D), (I-4-E), (I-4-F), (I-4-G) or (I-4-H), or a pharmaceutically acceptable salt, enantiomer, diastereomer, racemate, solvate, hydrate, polymorph, prodrug, or isotopic variant thereof, or mixture thereof, wherein

R₁ is —CF₃;

R₉ is H, —F, —Cl, —Br, -Me, —CF₃, —OMe, —OCF₃, —CN, —NHC(O)CH═CH₂ or —NH— cyclopropyl;

and other groups are as defined in above.

In a more specific embodiment, the present disclosure provides a compound of formula (I-5), (I-5-A), (I-5-B), (I-5-C), (I-5-D), (I-5-E), (I-5-F), (I-5-G) or (I-5-H), or a pharmaceutically acceptable salt, enantiomer, diastereomer, racemate, solvate, hydrate, polymorph, prodrug, or isotopic variant thereof, or mixture thereof, wherein

ring A is the following optionally substituted groups: C₃₋₇ cycloalkyl or C₆₋₁₀ aryl, wherein the substituent is selected from —F, —Cl, —Br, -Me, —OMe, —CF₃, —OCF₃, —CN, —NHMe, —P(O)Me₂, —NHC(O)CH₂CH₃, —C(O)CH═CH₂, —NHS(O)₂CH₂CH₃, —NH-cyclopropyl, —NHC(O)CH═CH₂ or —C(O)CH₂CH₃; ring A is alternatively the following groups:

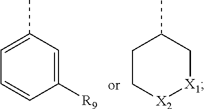

wherein

R₉ is H, —F, —Cl, —Br, -Me, —CF₃, —OMe, —OCF₃, —CN, —NHC(O)CH₂CH₃, —NHS(O)₂CH₂CH₃ or —NHC(O)CH═CH₂;

X₁ is —CH₂— or —N(R_{X1})—;

X₂ is —CH(R_{X2})— or —N(R_{X2})—;

wherein

R_{X1} is —C(O)CH₂CH₃ or —C(O)CH═CH₂;

R_{X2} is H, -Me, —OMe, —NHMe, —NHS(O)₂CH₂CH₃, —C(O)CH₂CH₃ or —NHC(O)CH₂CH₃;

[symbol] represents the point of attachment;

R₁ and R are taken together with the atoms to which they are attached to form optionally substituted 5- to 6-membered heterocyclyl, wherein the substituent is selected from halogen, oxo, -iPr, -Et, or phenyl, wherein the phenyl is mono- or poly-substituted with halogen; R₁ and R are alternatively taken together to form the following groups: —C(O)N(R_{N})C(O)— or —C(CH₃)═C(R_{N})C(O)—;

wherein

R_{N} is selected from -iPr, -Et or

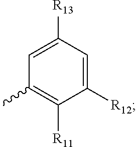

R₁₁ is —Cl, —Br;

R₁₂ is H or —F;

R₁₃ is H or —F;

[symbol] represents the point of attachment;

R₃ is H or —OMe;

R₄ is H or -Me;

and other groups are as defined in above.

In a more specific embodiment, the present disclosure provides a compound of formula (I-5-1) or (I′-5-1), or a pharmaceutically acceptable salt, enantiomer, diastereomer, racemate, solvate, hydrate, polymorph, prodrug, or isotopic variant thereof, or mixture thereof:

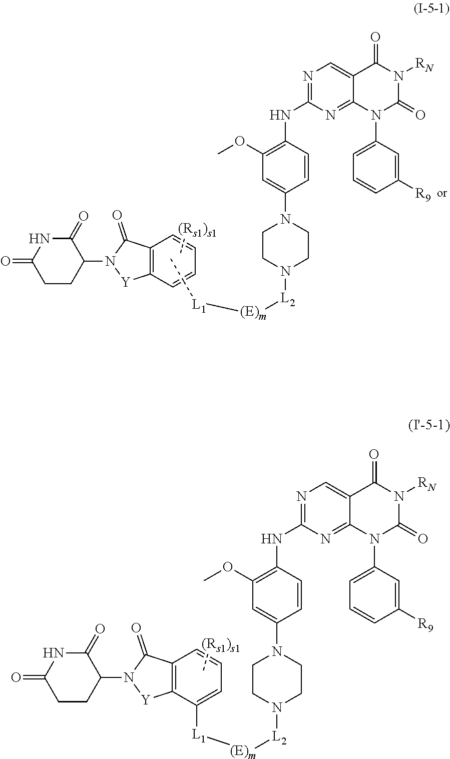

wherein

R₉ is —NHC(O)CH₂CH₃ or —NHC(O)CH═CH₂;

R_{N} is -iPr or -Et;

and other groups are as defined in above.

In a more specific embodiment, the present disclosure provides a compound of formula (I-5-2) or (I′-5-2), or a pharmaceutically acceptable salt, enantiomer, diastereomer, racemate, solvate, hydrate, polymorph, prodrug, or isotopic variant thereof, or mixture thereof:

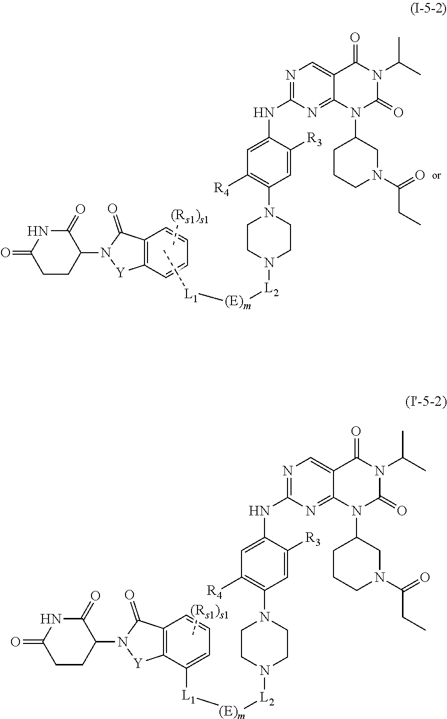

wherein

R₃ is H or —OMe;

R₄ is H or -Me;

and other groups are as defined in above.

In a more specific embodiment, the present disclosure provides a compound of formula (I-5-3) or (I′-5-3), or a pharmaceutically acceptable salt, enantiomer, diastereomer, racemate, solvate, hydrate, polymorph, prodrug, or isotopic variant thereof, or mixture thereof:

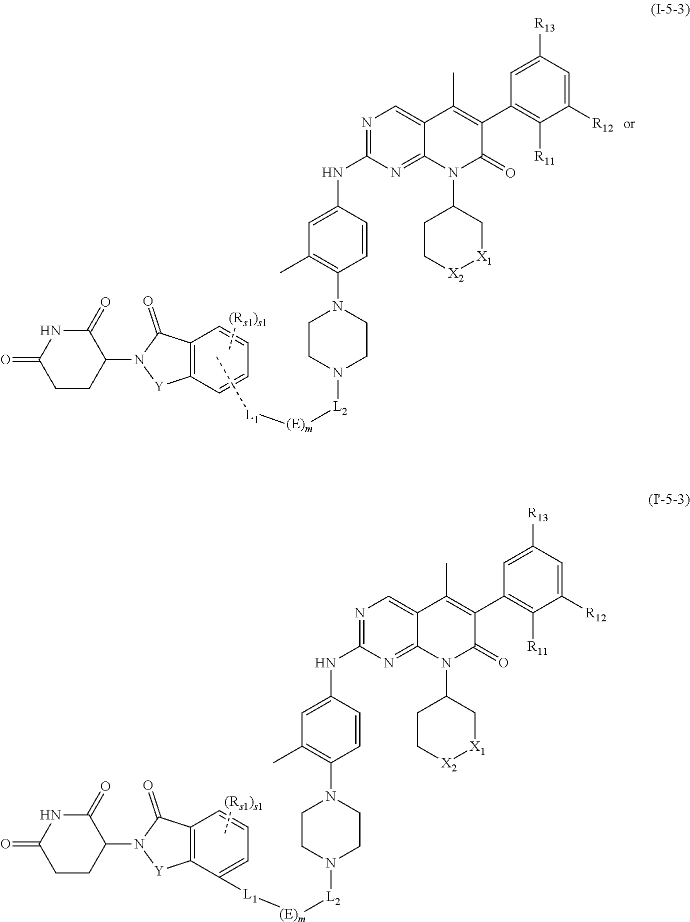

wherein

X₁ is —CH₂— or —N(R_{X1})—;

X₂ is —CH(R_{X2})— or —N(R_{X2})—;

wherein

R_{X1} is —C(O)CH₂CH₃;

R_{X2} is H, -Me, —OMe, —NHMe, —C(O)CH₂CH₃, —NHS(O)₂CH₂CH₃ or —NHC(O)CH₂CH₃;

R₁₁ is —Cl or —Br;

R₁₂ is H or —F;

R₁₃ is H or —F;

and other groups are as defined in above.

In a more specific embodiment, the present disclosure provides a compound of formula (I-6), (I-6-A), (I-6-B), (I-6-C), (I-6-D), (I-6-E), (I-6-F), (I-6-G) or (I-6-H), or a pharmaceutically acceptable salt, enantiomer, diastereomer, racemate, solvate, hydrate, polymorph, prodrug, or isotopic variant thereof, or mixture thereof, wherein

L is —O— or —NH—;

R₃ is H or —OMe;

R₄ is H or —F;

R₉ is —CF₃, —OMe, —OCF₃, —CN, —NHC(O)CH₂CH₃ or —NHC(O)CH═CH₂;

and other groups are as defined in above.

In a more specific embodiment, the present disclosure relates to a compound of all of the above formulas, or a pharmaceutically acceptable salt, enantiomer, diastereomer, racemate, solvate, hydrate, polymorph, prodrug, or isotopic variant thereof, or mixture thereof,

wherein

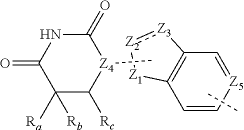

is selected from the following groups:

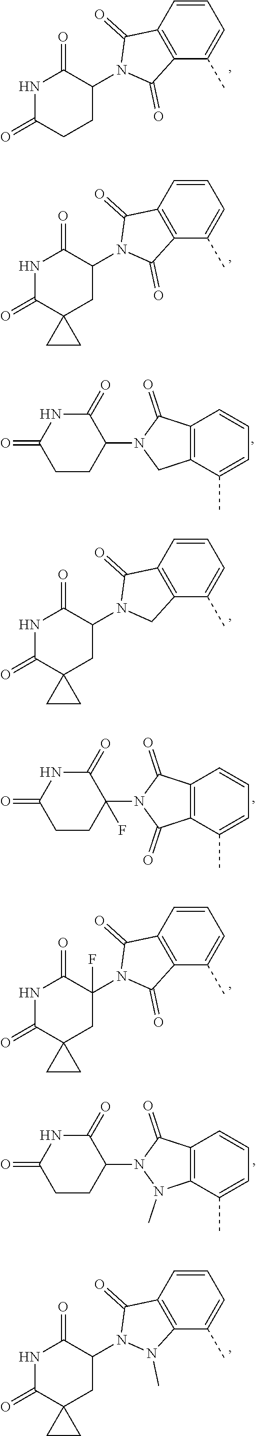

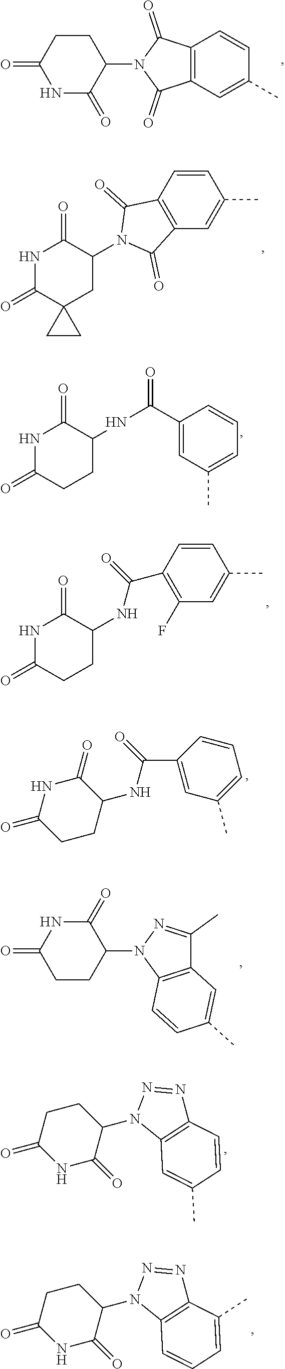

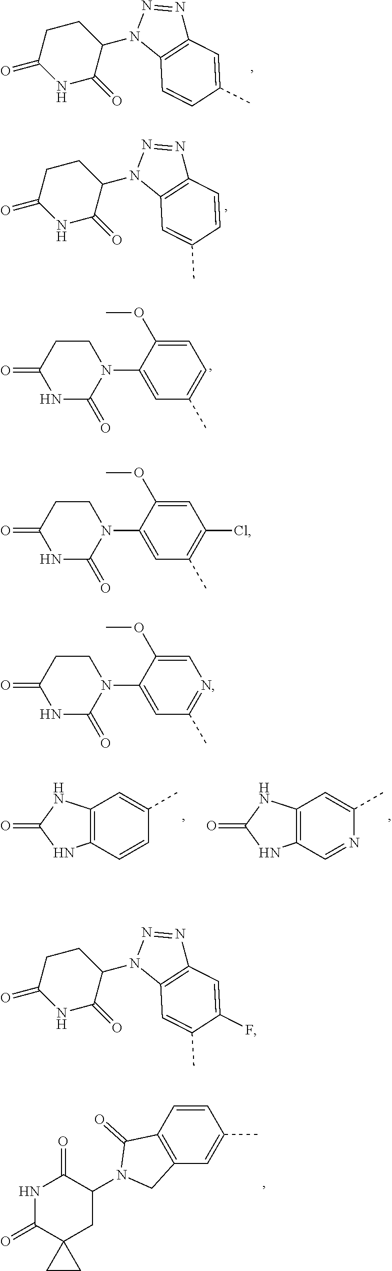

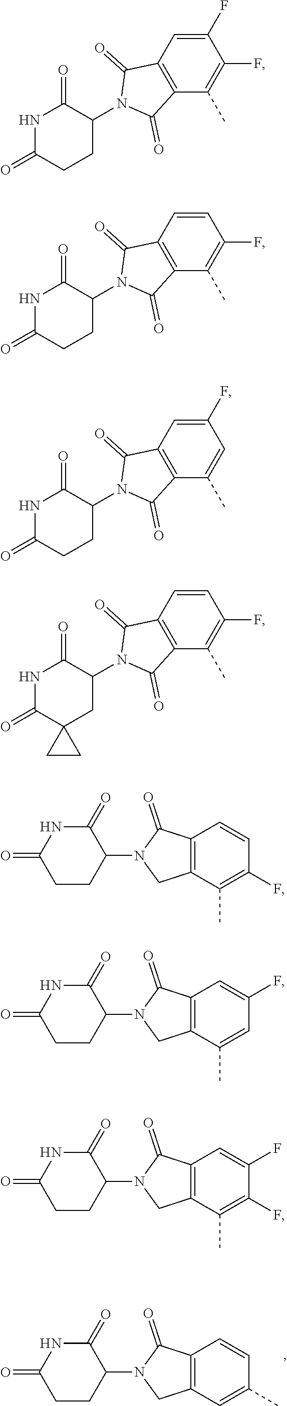

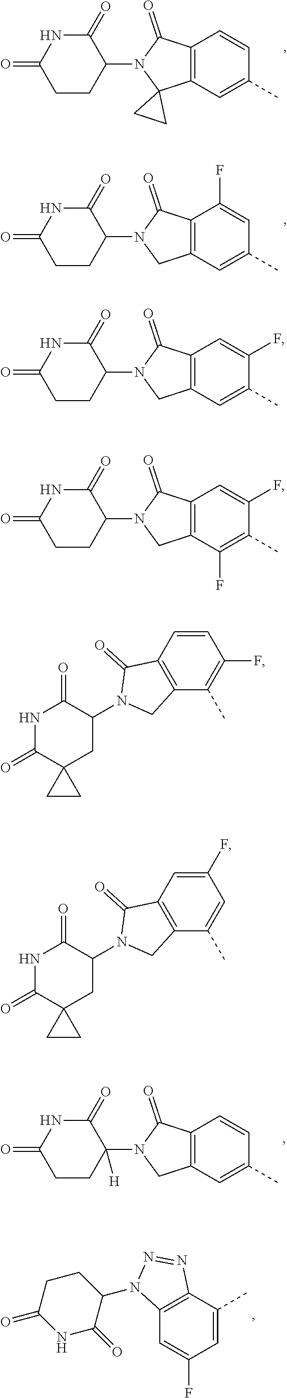

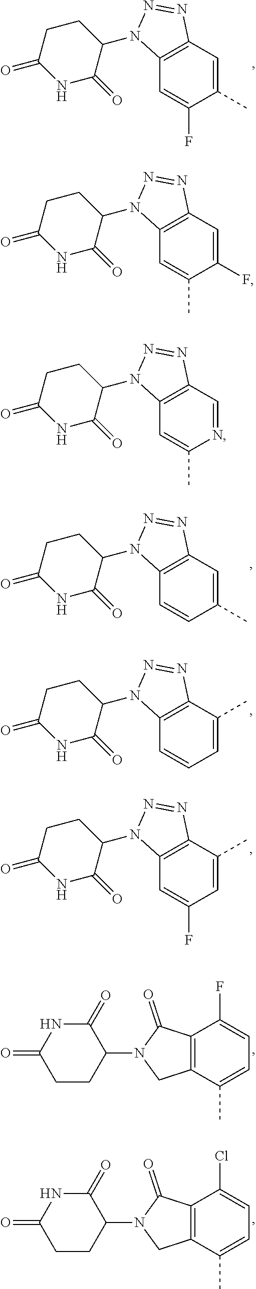

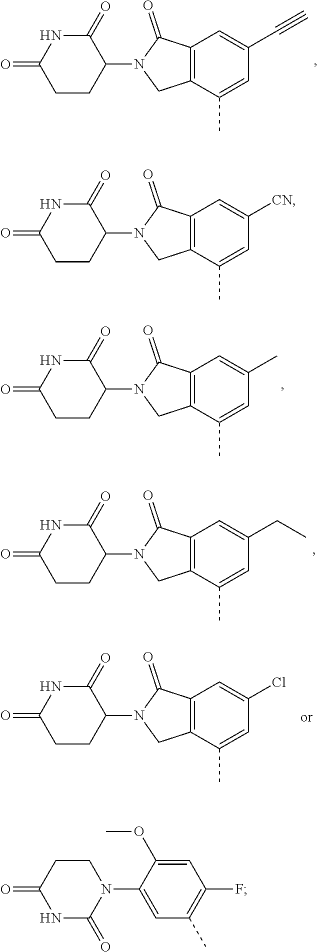

and one or more H atom(s) in the above groups can be substituted with D atom(s).

In a more specific embodiment, the present disclosure relates to a compound of all of the above formulas, or a pharmaceutically acceptable salt, enantiomer, diastereomer, racemate, solvate, hydrate, polymorph, prodrug, or isotopic variant thereof, or mixture thereof,

wherein L₁ and L₂ are independently selected from bond, —O—, —S—, —S(O)—, —S(O)₂—, —S(O)(═NH)—, —S(O)(═NMe)-,

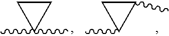

### —NH—, —N(Me)-,

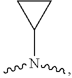

—N(CF₃)—, —CH₂—, —CH(OMe)-, —CH(Cl)—, —CH(F)—, —CF₂—, —CH(CF₃)—, —C(O)—, —CH₂CH₂—, —CH═CH—, —C≡C—, —OCH₂—, —CH₂O—, —SCH₂—, —CH₂S—, —S(O)CH₂—, —CH₂S(O)—, —S(O)₂CH₂—, —CH₂S(O)₂—, —NHCH₂—, —N(Me)CH₂—, —CH₂NH—, —CH₂N(Me)-, —C(O)CH₂—, —CH₂C(O)—, —C(O)CMe₂-, —CMe₂C(O)—, —OC(O)—, —C(O)O—, —SC(O)—, —C(O)S—, —NHC(O)—, —N(Me)C(O)—, —C(O)NH—, —C(O)N(Me)-, —S(O)═NH—, —NH═S(O)—, —N═S(O)Me-, —S(O)Me=N—, or;

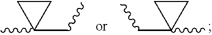

and one or more H atom(s) in the above groups can be substituted with D atom(s).

In a more specific embodiment, the present disclosure relates to a compound of all of the above formulas, or a pharmaceutically acceptable salt, enantiomer, diastereomer, racemate, solvate, hydrate, polymorph, prodrug, or isotopic variant thereof, or mixture thereof,

wherein E is selected from bond, —CH₂CH₂CH₂—, —CH₂CH═CH—, —CH═CHCH₂—, —CH₂C≡C—, —C≡CCH₂—, —CH₂CH₂C(O)—, —CH₂C(O)CH₂—, —C(O)CH₂CH₂—, —CH₂CH₂S(O)₂—, —CH₂S(O)₂CH₂—, —S(O)₂CH₂CH₂—, —C(O)CH═CH—, —C(O)C≡C—, —CH₂CH₂O—, —CH₂OCH₂—, —OCH₂CH₂—, —CH₂CH₂S—, —CH₂SCH₂—, —SCH₂CH₂—, —C(O)CH₂O—, —OCH₂C(O)—, —CH₂C(O)O—, —C(O)CH₂S—, —SCH₂C(O)—, —CH₂C(O)S—, —OC(O)CH₂—, —C(O)OCH₂—, —CH₂OC(O)—, —SC(O)CH₂—, —C(O)SCH₂—, —CH₂SC(O)—, —CH₂CH₂NH—, —CH₂NHCH₂—, —NHCH₂CH₂—, CH₂CH₂NMe-, —CH₂NMeCH₂—, —NMeCH₂CH₂—, —C(O)CH₂NH—, —NHCH₂C(O)—, —CH₂C(O)NH—, —NHC(O)CH₂—, —C(O)NHCH₂—, —CH₂NHC(O)—,

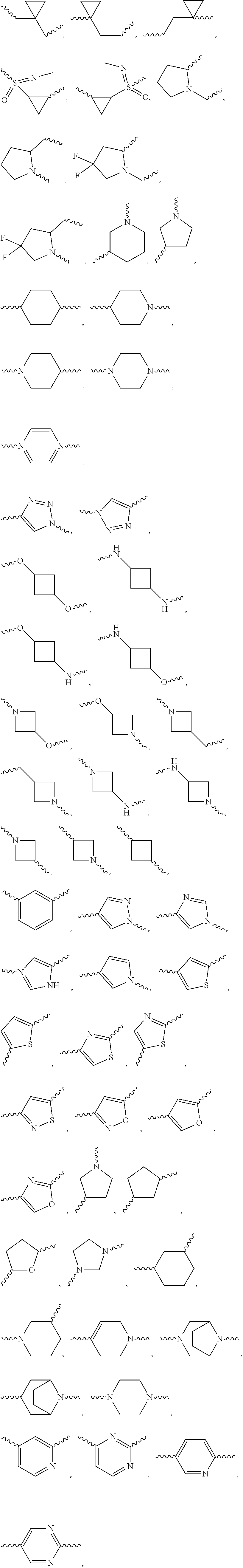

or two E moieties, or two E′ moieties can be taken together to form —CH₂CH₂OCH₂CH₂—, —OCH₂CH₂CH₂CH₂—, —CH₂CH₂CH₂CH₂O—,

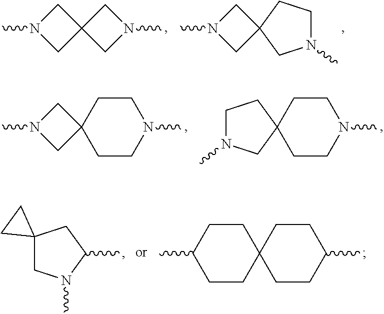

and one or more H atom(s) in the above groups can be substituted with D atom(s).

In a more specific embodiment, the present disclosure relates to a compound of all of the above formulas, or a pharmaceutically acceptable salt, enantiomer, diastereomer, racemate, solvate, hydrate, polymorph, prodrug, or isotopic variant thereof, or mixture thereof, wherein m is 0, 1, 4, 5, 6, 7, 8, 9 or 10; alternatively, m is 0, 1, 4, 5, 6, 7 or 8; alternatively, m is 0, 1, 4, 5 or 6; alternatively, m is 0, 1, 4 or 5.

In a more specific embodiment, the present disclosure relates to a compound of all of the above formulas, or a pharmaceutically acceptable salt, enantiomer, diastereomer, racemate, solvate, hydrate, polymorph, prodrug, or isotopic variant thereof, or mixture thereof, wherein the chain length of -L₁-(E)ₘ-L₂- is 4 to 14 bond lengths; alternatively, the chain length is less than 12 bond lengths; alternatively, the chain length is 5-10 bond lengths; alternatively, the chain length is 5, 6, 7, 8, 9 or 10 bond lengths.

In a more specific embodiment, the present disclosure relates to a compound, or a pharmaceutically acceptable salt, enantiomer, diastereomer, racemate, solvate, hydrate, polymorph, prodrug, or isotopic variant thereof, or mixture thereof, and the compound is selected from:

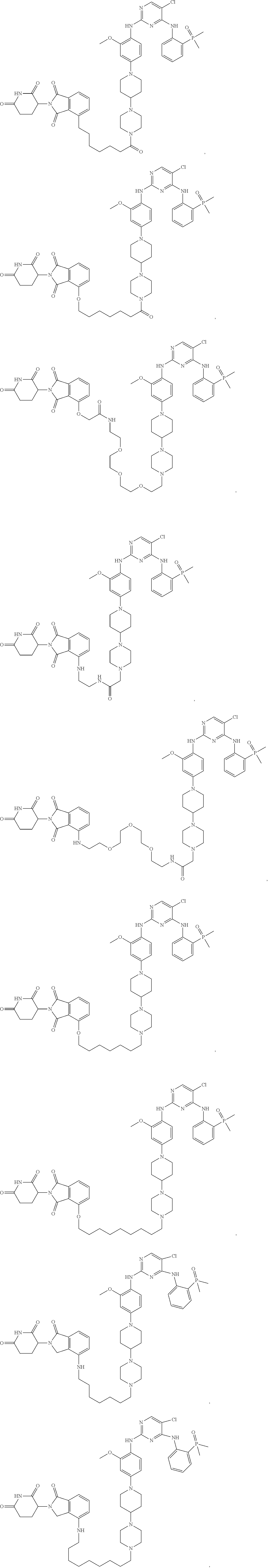

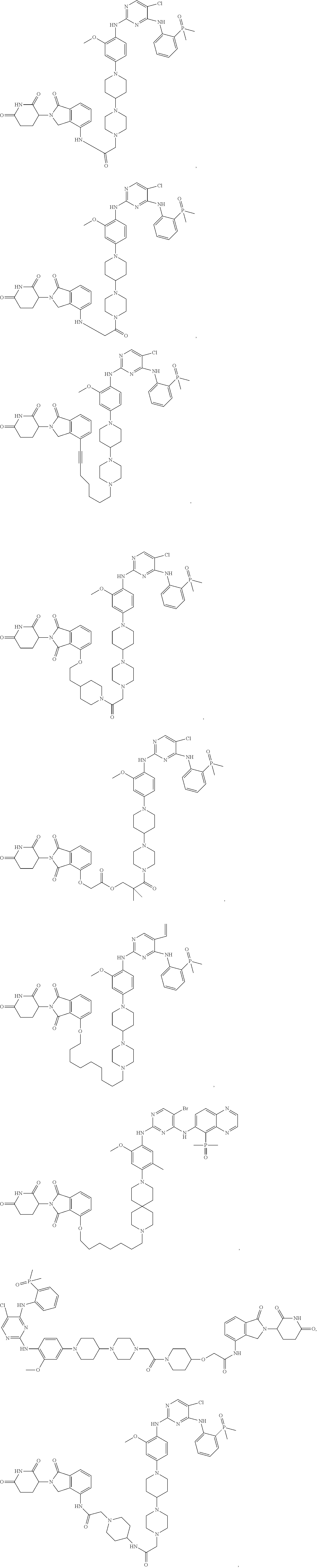

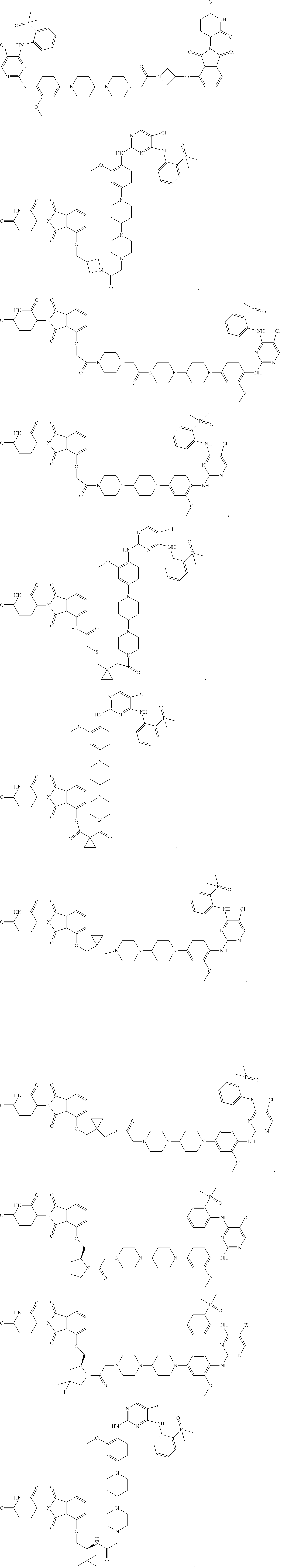

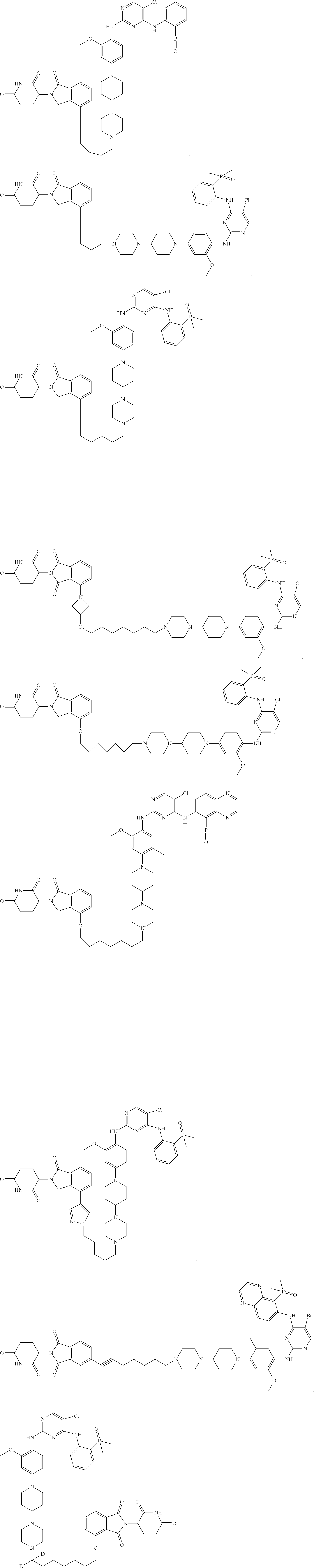

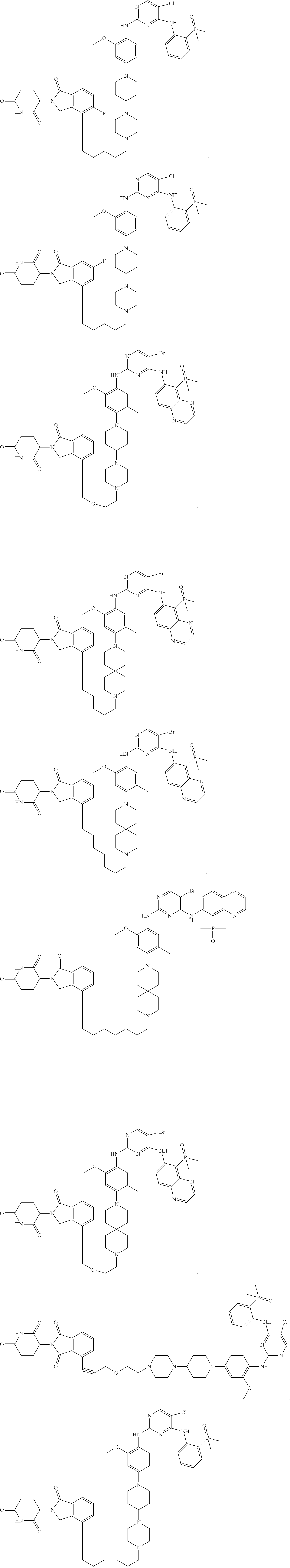

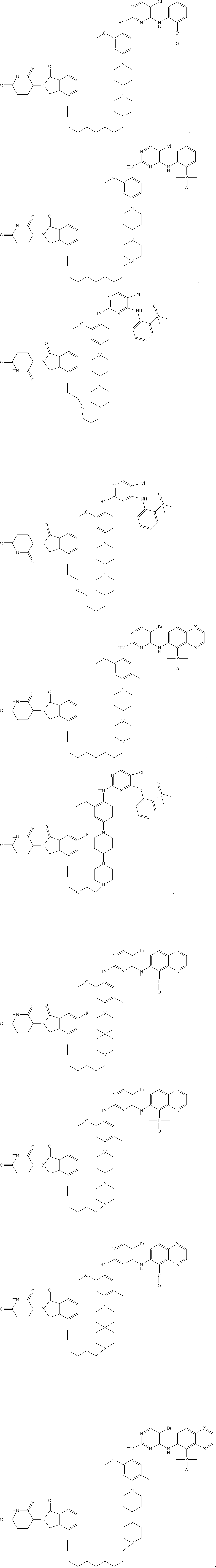

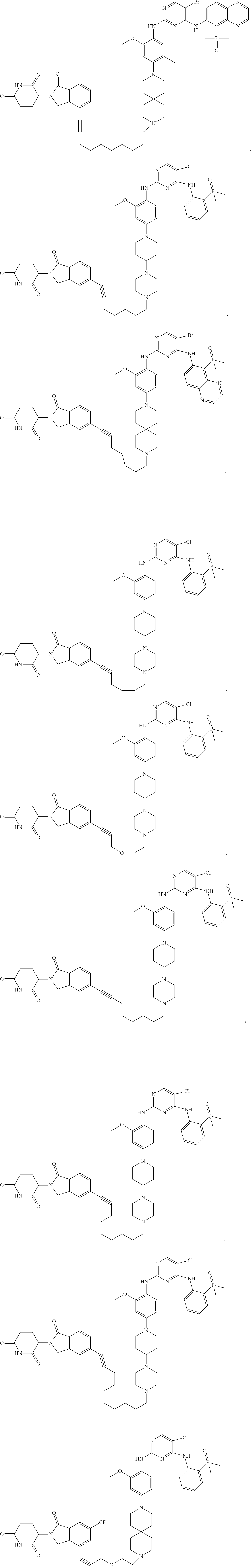

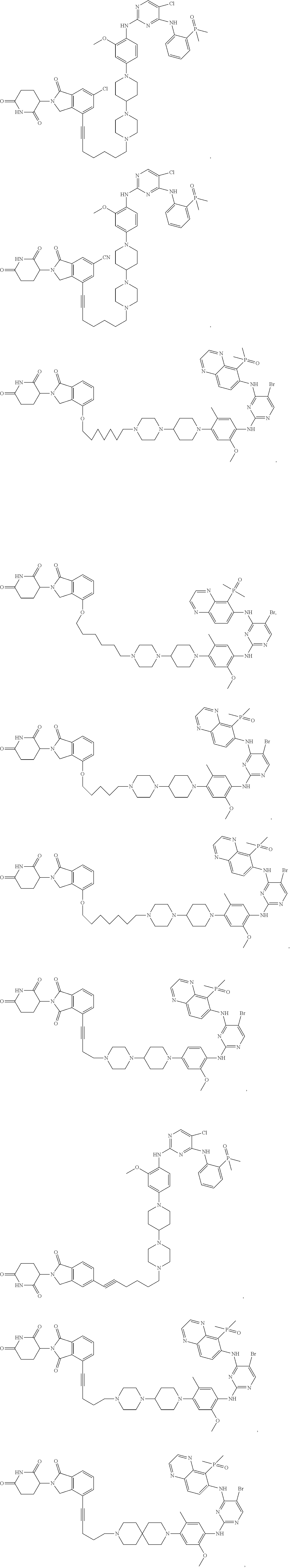

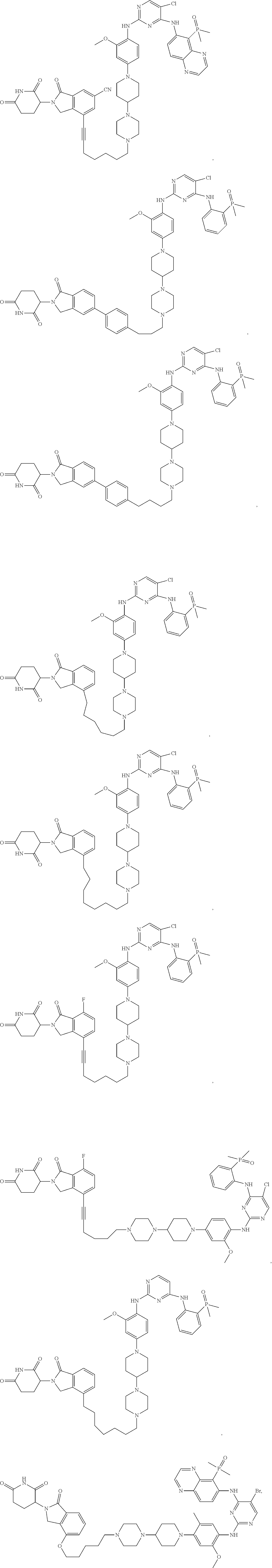

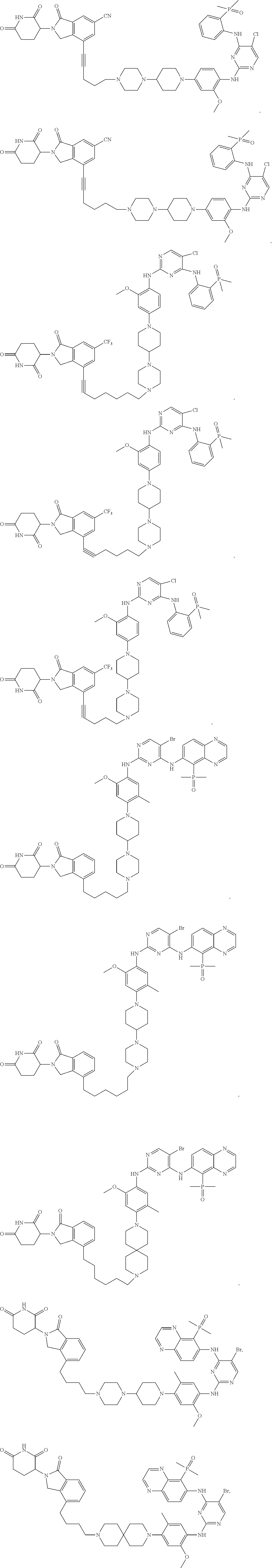

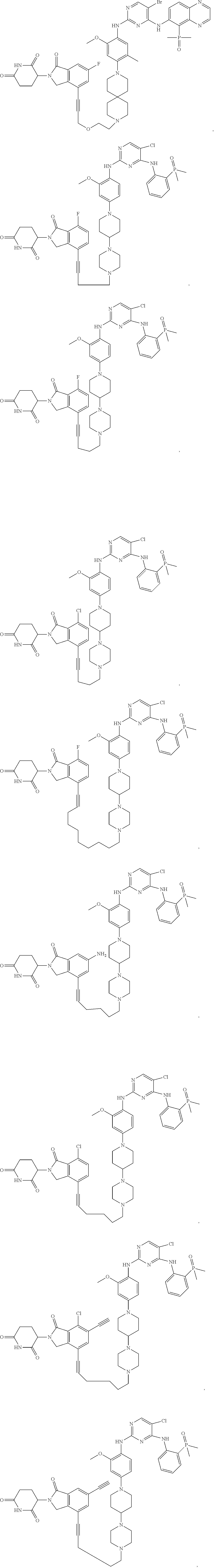

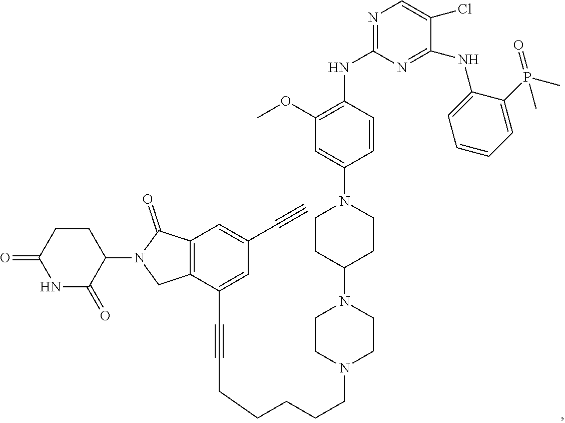 [SMILES: C#Cc1cc(C#CCCCCCN2CCN(C3CCN(c4ccc(Nc5ncc(Cl)c(Nc6ccccc6P(C)(C)=O)n5)c(OC)c4)CC3)CC2)c2c(c1)C(=O)N(C1CCC(=O)NC1=O)C2]

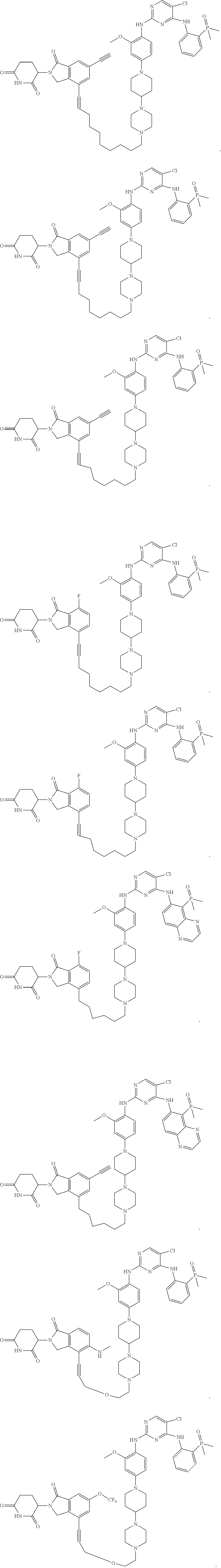

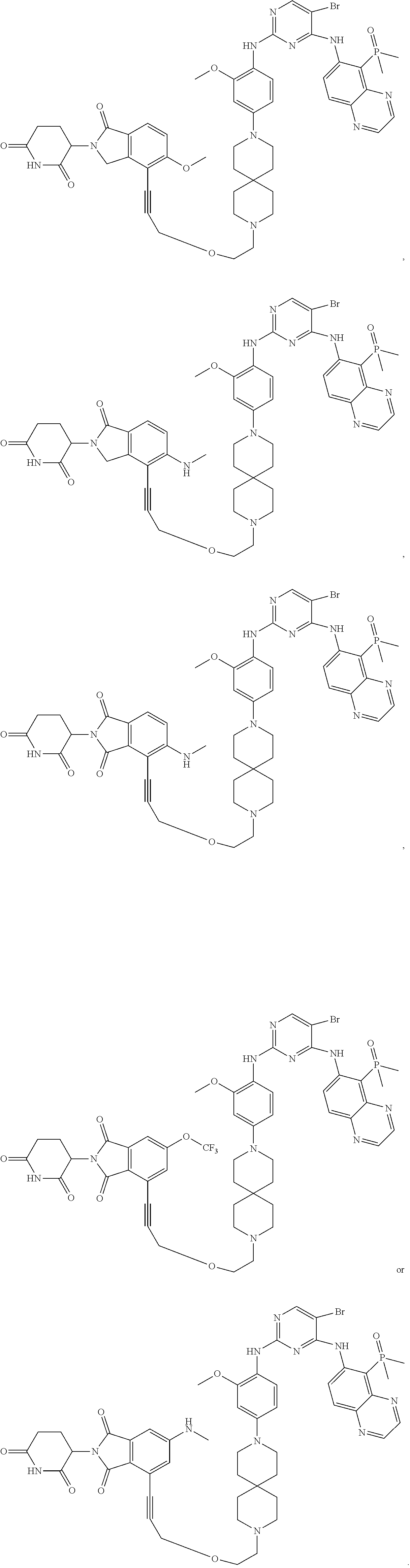

The compounds of the present disclosure may include one or more asymmetric centers, and thus may exist in a variety of stereoisomeric forms, for example, enantiomers and/or diastereomers. For example, the compounds of the present disclosure may be in the form of an individual enantiomer, diastereomer or geometric isomer (e.g., cis- and trans-isomers), or may be in the form of a mixture of stereoisomers, including racemic mixture and a mixture enriched in one or more stereoisomers. The isomers can be separated from the mixture by the methods known to those skilled in the art, including chiral high pressure liquid chromatography (HPLC) and the formation and crystallization of chiral salts; or alternative isomers can be prepared by asymmetric synthesis.

It will be understood by those skilled in the art that the organic compounds can form complexes with solvents in which they are reacted or from which they are precipitated or crystallized. These complexes are known as “solvates.” Where the solvent is water, the complex is known as “hydrate.” The present disclosure encompasses all solvates of the compounds of the present disclosure.

The term “solvate” refers to forms of a compound or a salt thereof, which are associated with a solvent, usually by a solvolysis reaction. This physical association may include hydrogen bonding. Conventional solvents include water, methanol, ethanol, acetic acid, DMSO, THF, diethyl ether, etc. The compounds described herein can be prepared, for example, in crystalline form, and can be solvated. Suitable solvates include pharmaceutically acceptable solvates and further include both stoichiometric solvates and non-stoichiometric solvates. In some cases, the solvates will be capable of isolation, for example, when one or more solvent molecules are incorporated into the crystal lattice of a crystalline solid. “Solvate” includes both solution-phase and isolatable solvates. Representative solvates include hydrates, ethanolates and methanolates.

The term “hydrate” refers to a compound that is associated with water. Generally, the number of water molecules contained in a hydrate of a compound is in a definite ratio to the number of the compound molecules in the hydrate. Therefore, hydrates of a compound can be represented, for example, by a general formula R-x H₂O, wherein R is the compound, and x is a number greater than 0. Given compounds can form more than one type of hydrates, including, for example, monohydrates (x is 1), lower hydrates (x is a number greater than 0 and smaller than 1, for example, hemihydrates (R.0.5H₂O)) and polyhydrates (x is a number greater than 1, for example, dihydrates (R.2H₂O) and hexahydrates (R.6H₂O)).

Compounds of the present disclosure may be in an amorphous or a crystalline form (polymorph). Furthermore, the compounds of the present disclosure may exist in one or more crystalline forms. Therefore, the present disclosure includes all amorphous or crystalline forms of the compounds of the present disclosure within its scope. The term “polymorph” refers to a crystalline form of a compound (or a salt, hydrate or solvate thereof) in a particular crystal packing arrangement. All polymorphs have the same elemental composition. Different crystalline forms generally have different X-ray diffraction patterns, infrared spectra, melting points, density, hardness, crystal shapes, optical and electrical properties, stability, and solubility. Recrystallization solvents, rate of crystallization, storage temperatures, and other factors may cause one crystalline form to dominate. Various polymorphs of a compound can be prepared by crystallization under different conditions.

The present disclosure also comprises compounds that are labeled with isotopes (isotope variants), which are equivalent to those described in formula (I), but one or more atoms are replaced by atoms having an atom mass or mass number that are different from that of atoms that are common in nature. Examples of isotopes which may be introduced into the compounds of the disclosure include isotopes of hydrogen, carbon, nitrogen, oxygen, phosphorus, sulfur, fluorine and chlorine, such as ²H, ³H, ¹³C, ¹¹C, ¹⁴C, ¹⁵N, ¹⁸O, ¹⁷O, ³¹P, ³²P, ³⁵, ¹⁸F and ³⁶Cl, respectively. Compounds of the present disclosure that comprise the above isotopes and/or other isotopes of other atoms, prodrugs thereof and pharmaceutically acceptable salts of said compounds or prodrugs all are within the scope of the present disclosure. Certain isotope-labeled compounds of the present disclosure, such as those incorporating radioactive isotopes (e.g., ³H and ¹⁴C), can be used for the measurement of the distribution of drug and/or substrate in tissue. Tritium, which is ³H and carbon-14, which is ¹⁴C isotope, are yet alternative, because they are easy to prepare and detect. Furthermore, replaced by heavier isotopes, such as deuterium, which is ²H, may provide therapeutic benefits due to the higher metabolic stability, such as prolonging the half-life in vivo or decreasing the dosage requirements, and thus may be alternative in some cases. Isotope-labeled compounds of formula (I) of the present disclosure and prodrugs thereof can be prepared generally by using readily available isotope-labeled reagents to replace non-isotope-labeled reagents in the following schemes and/or the procedures disclosed in the examples and preparation examples.

In addition, prodrugs are also included within the context of the present disclosure. The term “prodrug” as used herein refers to a compound that is converted into an active form that has medical effects in vivo by, for example, hydrolysis in blood. Pharmaceutically acceptable prodrugs are described in T. Higuchi and V. Stella, Prodrugs as Novel Delivery Systems, A.C.S. Symposium Series, Vol. 14, Edward B. Roche, ed., Bioreversible Carriers in Drug Design, American Pharmaceutical Association and Pergamon Press, 1987, and D. Fleisher, S. Ramon and H. Barbra “Improved oral drug delivery: solubility limitations overcome by the use of prodrugs”, Advanced Drug Delivery Reviews (1996) 19(2) 115-130, each of which are incorporated herein by reference.

The prodrugs are any covalently bonded compounds of the present disclosure, which release the parent compound in vivo when the prodrug is administered to a patient. Prodrugs are typically prepared by modifying functional groups in such a way that the modifications can be cleaved either by routine manipulation or decompose in vivo to yield the parent compound. Prodrugs include, for example, compounds of the present disclosure wherein the hydroxyl, amino or sulfhydryl groups are bonded to any group that, when administered to a patient, cleaves to form the hydroxyl, amino or sulfhydryl groups. Thus, representative examples of prodrugs include (but are not limited to) the acetate/acetamide, formate/formamide and benzoate/benzamide derivatives of the hydroxyl, amino or sulfhydryl functional groups of the compounds of formula (I). Furthermore, in the case of carboxylic acid (—COOH), esters such as methyl esters and ethyl esters, etc. can be employed. The ester itself may be active in their own and/or hydrolyzable under in vivo conditions in the human body. Suitable pharmaceutically acceptable in vivo hydrolysable ester groups include those groups that can readily break down in the human body to release the parent acids or salts thereof.

The present disclosure also provides a pharmaceutical formulation comprising a therapeutically effective amount of a compound of formula (I), or therapeutically acceptable salts thereof, and pharmaceutically acceptable carriers, diluents or excipients thereof. All of these forms belong to the present disclosure.

Pharmaceutical Compositions and Kits

In another aspect, the present disclosure provides a pharmaceutical composition comprising a compound of the present disclosure (also referred to as the “active ingredient”) and a pharmaceutically acceptable excipient. In certain embodiments, the pharmaceutical composition comprises an effective amount of the compound of the present disclosure. In certain embodiments, the pharmaceutical composition comprises a therapeutically effective amount of the compound of the present disclosure. In certain embodiments, the pharmaceutical composition comprises a prophylactically effective amount of the compound of the present disclosure.

A pharmaceutically acceptable excipient for use in the present disclosure refers to a non-toxic carrier, adjuvant or vehicle which does not destroy the pharmacological activity of the compound formulated together. Pharmaceutically acceptable carriers, adjuvants, or vehicles that may be used in the compositions of the present disclosure include, but are not limited to, ion exchangers, alumina, aluminum stearate, lecithin, serum proteins (e.g., human serum albumin), buffer substances (such as phosphate), glycine, sorbic acid, potassium sorbate, a mixture of partial glycerides of saturated plant fatty acids, water, salt or electrolyte (such as protamine sulfate), disodium hydrogen phosphate, potassium hydrogen phosphate, sodium chloride, zinc salt, silica gel, magnesium trisilicate, polyvinyl pyrrolidone, cellulose-based materials, polyethylene glycol, sodium carboxymethyl cellulose, polyacrylate, wax, polyethylene-polyoxypropylene block polymers, polyethylene glycol and lanolin.

The present disclosure also includes kits (e.g., pharmaceutical packs). Kits provided may include a compound disclosed herein, other therapeutic agents, and a first and a second containers (e.g., vials, ampoules, bottles, syringes, and/or dispersible packages or other materials) containing the compound disclosed herein or other therapeutic agents. In some embodiments, kits provided can also optionally include a third container containing a pharmaceutically acceptable excipient for diluting or suspending the compound disclosed herein and/or other therapeutic agent. In some embodiments, the compound disclosed herein provided in the first container and the other therapeutic agents provided in the second container is combined to form a unit dosage form.

Administration

The pharmaceutical composition provided by the present disclosure can be administered by a variety of routes including, but not limited to, oral administration, parenteral administration, inhalation administration, topical administration, rectal administration, nasal administration, oral administration, vaginal administration, administration by implant or other means of administration. For example, parenteral administration as used herein includes subcutaneous administration, intradermal administration, intravenous administration, intramuscular administration, intra-articular administration, intraarterial administration, intrasynovial administration, intrasternal administration, intracerebroventricular administration, intralesional administration, and intracranial injection or infusion techniques.

Generally, the compounds provided herein are administered in an effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

When used to prevent the disorder disclosed herein, the compounds provided herein will be administered to a subject at risk for developing the condition, typically on the advice and under the supervision of a physician, at the dosage levels described above. Subjects at risk for developing a particular condition generally include those that have a family history of the condition, or those who have been identified by genetic testing or screening to be particularly susceptible to developing the condition.

The pharmaceutical compositions provided herein can also be administered chronically (“chronic administration”). Chronic administration refers to administration of a compound or pharmaceutical composition thereof over an extended period of time, e.g., for example, over 3 months, 6 months, 1 year, 2 years, 3 years, 5 years, etc, or may be continued indefinitely, for example, for the rest of the subject's life. In certain embodiments, the chronic administration is intended to provide a constant level of the compound in the blood, e.g., within the therapeutic window over the extended period of time.

The pharmaceutical compostions of the present disclosure may be further delivered using a variety of dosing methods. For example, in certain embodiments, the pharmaceutical composition may be given as a bolus, e.g., in order to raise the concentration of the compound in the blood to an effective level. The placement of the bolus dose depends on the systemic levels of the active ingredient desired throughout the body, e.g., an intramuscular or subcutaneous bolus dose allows a slow release of the active ingredient, while a bolus delivered directly to the veins (e.g., through an IV drip) allows a much faster delivery which quickly raises the concentration of the active ingredient in the blood to an effective level. In other embodiments, the pharmaceutical composition may be administered as a continuous infusion, e.g., by IV drip, to provide maintenance of a steady-state concentration of the active ingredient in the subject's body. Furthermore, in still yet other embodiments, the pharmaceutical composition may be administered as first as a bolus dose, followed by continuous infusion.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or alternatively from about 1 to about 40% by weight) with the remainder being various vehicles or excipients and processing aids helpful for forming the desired dosing form.

With oral dosing, one to five and especially two to four and typically three oral doses per day are representative regimens. Using these dosing patterns, each dose provides from about 0.01 to about 20 mg/kg of the compound provided herein, with alternative doses each providing from about 0.1 to about 10 mg/kg, and especially about 1 to about 5 mg/kg.

Transdermal doses are generally selected to provide similar or lower blood levels than are achieved using injection doses, generally in an amount ranging from about 0.01 to about 20% by weight, alternatively from about 0.1 to about 20% by weight, alternatively from about 0.1 to about 10% by weight, and still alternatively from about 0.5 to about 15% by weight.

Injection dose levels range from about 0.1 mg/kg/hour to at least 10 mg/kg/hour, all for from about 1 to about 120 hours and especially 24 to 96 hours. A preloading bolus of from about 0.1 mg/kg to about 10 mg/kg or more may also be administered to achieve adequate steady state levels. The maximum total dose is not expected to exceed about 2 g/day for a 40 to 80 kg human patient.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable excipients known in the art. As before, the active compound in such compositions is typically a minor component, often being from about 0.05 to 10% by weight with the remainder being the injectable excipient and the like.

Transdermal compositions are typically formulated as a topical ointment or cream containing the active ingredient(s). When formulated as a ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or Formulation. All such known transdermal formulations and ingredients are included within the scope provided herein.

The compounds provided herein can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pa., which is incorporated herein by reference.

The compounds of the present disclosure can also be administered in sustained release forms or from sustained release drug delivery systems. A description of representative sustained release materials can be found in Remington's Pharmaceutical Sciences.

The present disclosure also relates to the pharmaceutically acceptable formulations of a compound of the present disclosure. In one embodiment, the formulation comprises water. In another embodiment, the formulation comprises a cyclodextrin derivative. The most common cyclodextrins are α-, β- and γ-cyclodextrins consisting of 6, 7 and 8 α-1,4-linked glucose units, respectively, optionally comprising one or more substituents on the linked sugar moieties, which include, but are not limited to, methylated, hydroxyalkylated, acylated, and sulfoalkylether substitution. In certain embodiments, the cyclodextrin is a sulfoalkyl ether β-cyclodextrin, e.g., for example, sulfobutyl ether O-cyclodextrin, also known as Captisol. See, e.g., U.S. Pat. No. 5,376,645. In certain embodiments, the formulation comprises hexapropyl-γ-cyclodextrin (e.g., 10-50% in water).

Treatment

As stated herein, it is known that EGFR kinase have roles in tumourigenesis as well as numerous other diseases. We have found that the compounds of the present disclosure possess potent anti-tumour activity which it is believed is afforded by way of inhibition of EGFR kinase.

The compounds of the present disclosure are of value as anti-tumour agents. Particularly, the compounds of the present disclosure are of value as anti-proliferative, apoptotic and/or anti-invasive agents in the containment and/or treatment of solid and/or liquid tumour disease. Particularly, the compounds of the present disclosure are expected to be useful in the prevention or treatment of those tumours which are sensitive to inhibition of EGFR. Further, the compounds of the present disclosure are expected to be useful in the prevention or treatment of those tumours which are mediated alone or in part by EGFR. The compounds may thus be used to produce an EGFR enzyme inhibitory effect in a warm-blooded animal in need of such treatment.

As stated herein, inhibitors of EGFR kinase should be of therapeutic value for the treatment of cancer, such as ovarian cancer, cervical cancer, colorectal cancer, breast cancer, pancreatic cancer, glioma, glioblastoma, melanoma, prostate cancer, leukemia, lymphoma, non-Hodgkin's lymphoma, gastric cancer, lung cancer, hepatocellular cancer, stomach cancer, gastrointestinal stromal tumor (GIST), thyroid cancer, cancer of bile duct, endometrial cancer, kidney cancer, anaplastic large cell lymphoma, acute myeloid leukemia (AML), multiple myeloma, melanoma, and mesothelioma.

Anti-cancer effects which are accordingly useful in the treatment of cancer in a patient include, but are not limited to, anti-tumour effects, the response rate, the time to disease progression and the survival rate. Anti-tumour effects of a method of treatment of the present disclosure include but are not limited to, inhibition of tumour growth, tumour growth delay, regression of tumour, shrinkage of tumour, increased time to regrowth of tumour on cessation of treatment, slowing of disease progression. Anti-cancer effects include prophylactic treatment as well as treatment of existing disease.

A EGFR kinase inhibitor, or a pharmaceutically acceptable salt thereof, may also be useful for the treatment patients with cancers, including, but not limited to, haematologic malignancies such as leukaemia, multiple myeloma, lymphomas such as Hodgkin's disease, non-Hodgkin's lymphomas (including mantle cell lymphoma), and myelodysplastic syndromes, and also solid tumours and their metastases such as breast cancer, lung cancer (non-small cell lung cancer (NSCL), small cell lung cancer (SCLC), squamous cell carcinoma), endometrial cancer, tumours of the central nervous system such as gliomas, dysembryoplastic neuroepithelial tumour, glioblastoma multiforme, mixed gliomas, medulloblastoma, retinoblastoma, neuroblastoma, germinoma and teratoma, cancers of the gastrointestinal tract such as gastric cancer, oesophagal cancer, hepatocellular (liver) carcinoma, cholangiocarcinomas, colon and rectal carcinomas, cancers of the small intestine, pancreatic cancers, cancers of the skin such as melanomas (in particular metastatic melanoma), thyroid cancers, cancers of the head and neck and cancers of the salivary glands, prostate, testis, ovary, cervix, uterus, vulva, bladder, kidney (including renal cell carcinoma, clear cell and renal oncocytoma), squamous cell carcinomas, sarcomas such as osteosarcoma, chondrosarcoma, leiomyosarcoma, soft tissue sarcoma, Ewing's sarcoma, gastrointestinal stromal tumour (GIST), Kaposi's sarcoma, and paediatric cancers such as rhabdomyosarcomas and neuroblastomas.

The effective dose of the compound of the present disclosure is usually at an average daily dose of 0.01 mg to 50 mg compound/kg of patient weight, alternatively 0.1 mg to 25 mg compound/kg of patient weight, in single or multiple administrations. Generally, the compound of the present disclosure can be administered to the patient who needs this treatment in the daily dose range of about 1 mg to about 3500 mg per patient, alternatively 10 mg to 1000 mg. For example, the daily dose per patient can be 10, 20, 30, 40, 50, 60, 70, 80, 90, 100, 150, 200, 250, 300, 350, 400, 500, 600, 700, 800, 900 or 1000 mg. It can be administered once or several times a day, weekly (or several days apart) or on an intermittent schedule. For example, on a weekly basis (e.g. every Monday), the compound can be administered one or more times a day, variably for several weeks, for example 4-10 weeks. Or, the compound may be administered daily for several days (e.g. 2-10 days), and then a few days (e.g. 1-30 days) without administering the compound, repeating the cycle arbitrarily or repeating a given number of times, e.g. 4-10 cycles. For example, the compound of the present disclosure can be administered daily for 5 days, and then interrupted for 9 days, and then administered daily for 5 days, then interrupted for 9 days, and so on, repeating the cycle arbitrarily or repeating 4-10 times in total.

Combination Therapy

The treatment defined herein may be applied as a sole therapy or may involve, in addition to the compounds of the present disclosure, conventional surgery or radiotherapy or chemotherapy. Accordingly, the compounds of the present disclosure can also be used in combination with existing therapeutic agents for the treatment of cancer.

In addition to the compound disclosed herein, conventional surgery, radiotherapy, chemotherapy, or immunotherapy can be used for the treatment. Such chemotherapy can be administered simultaneously, sequentially or separately with the compound disclosed herein and may include one or more of the following categories of anti-tumour agents:

(i) antiproliferative/antineoplastic drugs and combinations thereof, as used in medical oncology, such as alkylating agents (for example czs-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);

(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5a-reductase such as finasteride;

(iii) anti-invasion agents [for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline [AZD0530 (saracatinib)], N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825) and bosutinib (SKI-606), and metalloproteinase inhibitors like marimastat, inhibitors of urokinase plasminogen activator receptor function or antibodies to heparanase];

(iv) inhibitors of growth factor function: for example such inhibitors include growth factor antibodies and growth factor receptor antibodies (for example the anti-erbB2 antibody trastuzumab [Herceptin], the anti-EGFR antibody panitumumab and the anti-erbBl antibody cetuximab [Erbitux, C225]); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)-quinazolin-4-amine (gefitinib, ZD 1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib (BAY 43-9006), tipifarnib (R¹ 15777) and lonafarnib (SCH66336)), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor (insulinlike growth factor) kinase inhibitors; aurora kinase inhibitors (for example AZD1152, PH739358, VX—680, MLN8054, R⁷⁶³, MP235, MP529, VX—528 and AX39459) and cyclin dependent kinase inhibitors such as CDK2 and/or CDK4 inhibitors;

(v) antiangiogenic agents such as those which inhibit the effects of vascular endothelial growth factor, [for example the anti-human vascular endothelial cell growth factor antibody bevacizumab (Avastin) and for example, a VEGF receptor tyrosine kinase inhibitor such as vandetanib (ZD6474), vatalanib (PTK787), sunitinib (SUl 1248), axitinib (AG-013736), pazopanib (GW 786034) and 4-(4-fiuoro-2-methylindol-5-yloxy)-6-methoxy-7-(3-pyrrolidin-1-ylpropoxy)quinazoline (AZD2171) and compounds that work by other mechanisms (for example linomide, inhibitors of integrin avP3 function and angiostatin)];

(vi) vascular damaging agents such as combretastatin A4;

(vii) an endothelin receptor antagonist, for example zibotentan (ZD4054) or atrasentan;

(viii) antisense therapies, for example those which are directed to the targets listed above, such as ISIS 2503, an anti-ras antisense;

(ix) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and

(x) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches usingcytokine-transfected tumour cell lines, approaches using anti-idiotypic antibodies, approaches to decrease the function of immune suppressive cells such as regulatory T cells, myeloid-derived suppressor cells or IDO (indoleamine 2,3,-deoxygenase)-expressing dendritic cells, and approaches using cancer vaccines consisting of proteins or peptides derived from tumour-associated antigens such as NY-ESO-1, MAGE-3, WTl or Her2/neu.

### EXAMPLES

The materials or reagents used herein are commercially available or are prepared by synthetic methods generally known in the art.

### Preparation of Intermediates

### Preparation of 2-(2,6-dioxopiperidin-3-yl)-4-hydroxyisoindoline-1,3-dione (A2-1)

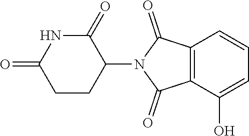 [SMILES: O=C1CCC(N2C(=O)c3cccc(O)c3C2=O)C(=O)N1]

4-Hydroxyisobenzofuran-1,3-dione (5.0 g, 30.5 mmol), 3-aminopiperidine-2,6-dione (6.85 g, 41.8 mmol) and sodium acetate (4.1 g, 50.0 mmol) were dispersed in 100 mL of CH₃COOH, and the mixture was stirred and refluxed at 140° C. under the protection of nitrogen for 8 h. The mixture was cooled to room temperature and concentrated under reduced pressure to remove CH₃COOH, and then 200 mL of water was added to the residue. The mixture was then slurried with stirring for 2 h. The mixture was filtrated with suction. The solid product was rinsed twice with water, and dried with baking to afford 7.4 g of off-white solid product. Yield: 89.15%. LCMS: [M+H]+=275. ¹H-NMR: (400 MHz, DMSO-d₆) δ 11.164 (s, 1H), 11.080 (s, 1H), 7.675 (dd, J=7.2 Hz, 8.4 Hz, 1H), 7.329 (dd, J=7.2 Hz, 26.4 Hz, 2H), 5.094 (dd, J=5.2 Hz, 12.8 Hz, 1H), 2.919-2.840 (m, 1H), 2.619-2.530 (m, 2H), 2.048-1.989 (m, 1H).

### Preparation of 2-(2,6-dioxopiperidin-3-yl)-4-fluoroisoindoline-1,3-dione (A6-1)

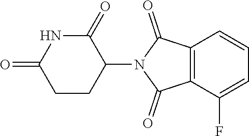 [SMILES: O=C1CCC(N2C(=O)c3cccc(F)c3C2=O)C(=O)N1]

The preparation method was the same as the preparation of 2-(2,6-dioxopiperidin-3-yl)-4-hydroxyisoindoline-1,3-dione, and 4-fluoroisobenzofuran-1,3-dione was used as raw material to afford a white solid. LCMS: [M+H]=277.

### Preparation of 4-bromo-2-(2,6-dioxopiperidin-3-yl)isoindoline-1,3-dione (A1-1)

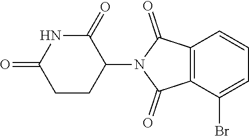 [SMILES: O=C1CCC(N2C(=O)c3cccc(Br)c3C2=O)C(=O)N1]

The preparation method was the same as the preparation of 2-(2,6-dioxopiperidin-3-yl)-4-hydroxyisoindoline-1,3-dione, and 4-bromoisobenzofuran-1,3-dione was used as raw material to afford a white solid. LCMS: [M+H]=337, 339.

### Preparation of 2-(4-(1-(4-((5-chloro-4-((2-(dimethylphosphoryl)phenyl)amino)pyrimidin-2-yl)amino)-3-methoxyphenyl)piperidin-4-yl)piperazin-1-yl)acetic acid (A6-5)

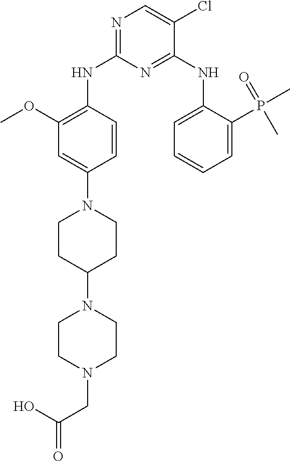

(2-((5-Chloro-2-((2-methoxy-4-(4-(piperazin-1-yl)piperidin-1-yl)phenyl)amino)pyrimidin-4-yl)amino)phenyl)dimethyl phosphorus oxide (300 mg, 0.527 mmol), potassium carbonate (145 mg, 1.054 mmol) and tert-butyl bromoacetate (113.04 mg, 0.527 mmol) were added successively to a 100 mL three-necked flask equipped with a condenser and dissolved in DMF (5 mL). The mixture was heated to 60° C., and reacted with stirring for 3 h. The mixture was cooled to room temperature, then diluted by adding 20 mL of water, and extracted with ethyl acetate for three times (20 mL for each time). The organic phases were combined, washed with saturated brine for three times, dried with anhydrous sodium sulfate, and concentrated under reduced pressure to afford a crude product, which was separated by silica gel column chromatography to afford 120 mg of yellow solid. The solid afforded above was dissolved in 1,4-dioxane (5 mL), 2 mL of HCl/1,4-dioxane solution (4 mol/L) was added, and the mixture was stirred at room temperature for 2 h. The reaction result was detected by LCMS. The mixture was basically the title product, and it was concentrated under reduced pressure to afford 112 mg of yellow solid. Yield: 34%. LCMS: [M+H]⁺=628.

### Preparation of 3-(4-bromo-1-oxoisoindolin-2-yl)piperidine-2,6-dione (C1-1)

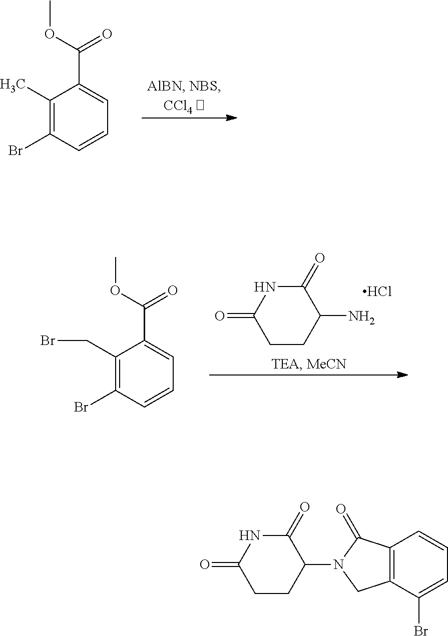 [SMILES: COC(=O)c1cccc(Br)c1C ; COC(=O)c1cccc(Br)c1CBr ; NC1CCC(=O)NC1=O ; Cl ; O=C1CCC(N2Cc3c(Br)cccc3C2=O)C(=O)N1]

Methyl 3-bromo-2-methylbenzoate (1.14 g, 5.0 mmol) was dissolved in 20.0 mL of CCl₄. NBS (1.34 g, 7.5 mmol) and AIBN (164 mg, 1.0 mmol) were addded under nitrogen protection. The mixture was heated to 85° C., and reacted under reflux for 20 h. TLC showed that there was no raw material remaining. The mixture was cooled to room temperature, then filtered with suction and concentrated under reduced pressure to afford a crude product, which was purified by Flash to afford 1.35 g of light-yellow oily product. The oily compound (1.35 g, 4.41 mmol) and 3-aminopiperidine-2,6-dione hydrochloride (941 mg, 5.74 mmol) were dispersed in 25.0 mL of anhydrous MeCN, and TEA (580 mg, 5.74 mmol) was added. The mixture was heated to 80° C., and reacted under reflux for 16 h. LCMS showed that the reaction was completed. The mixture was cooled to room temperature, and then filtrated with suction. The filter cake was washed with MeCN for three times, and dried with baking to afford compound C1-1 (1.31 g, yield: 92.3%). LCMS: [M+H]⁺=323, 325.

### Preparation of 2-(4-(1-(4-((5-chloro-4-((2-(dimethylphosphoryl)phenyl)amino)pyrimidin-2-yl)amino)-3-methoxyphenyl)piperidin-4-yl)piperazin-1-yl)-N-(piperidin-4-yl)acetamide (C₈₈₋₄)

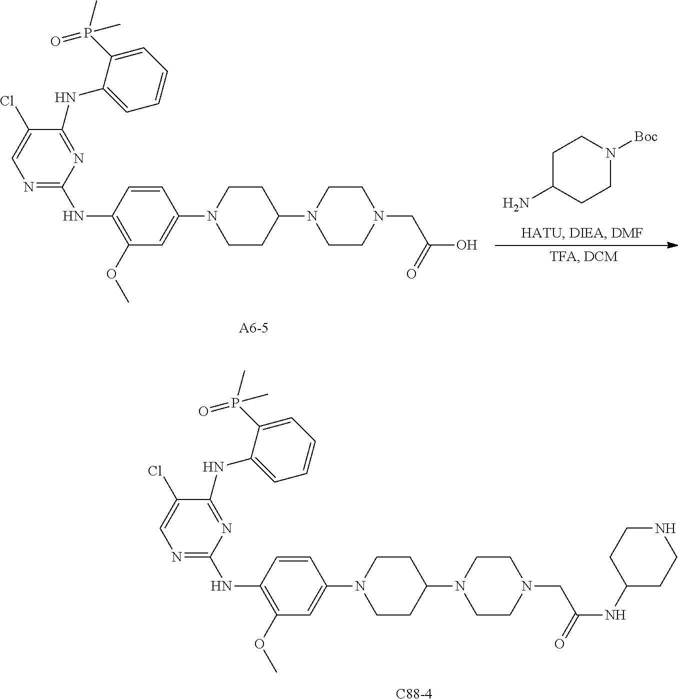 [SMILES: "C88-4" ; COc1cc(N2CCC(N3CCN(CC(=O)O)CC3)CC2)ccc1Nc1ncc(Cl)c(Nc2ccccc2P(C)(C)=O)n1 ; COc1cc(N2CCC(N3CCN(CC(=O)NC4CCNCC4)CC3)CC2)ccc1Nc1ncc(Cl)c(Nc2ccccc2P(C)(C)=O)n1 ; CC(C)(C)OC(=O)N1CCC(N)CC1]

Compound A6-5 (75 mg, 0.12 mmol), HATU (45 mg, 0.118 mmol), and DIEA (77 mg, 0.60 mmol) were dissolved in 2.5 mL of DMF, and the mixture was stirred at room temperature under nitrogen protection for 1 h. Then, tert-butyl 4-aminopiperidine-1-carboxylate (26 mg, 0.13 mmol) was added and the mixture was further stirred for 2.5 h. The solvent was removed under reduced pressure, and the residue was dissolved in dichloromethane (4.0 mL). TFA (1.0 mL) was added dropwise, and the mixture was stirred at room temperature for 2 h. The solvent was removed under reduced pressure, and the residue was separated by thin layer chromatography to afford compound C88-4. LCMS: [M+H]⁺=710.

### Preparation of 3-(5-bromo-1-oxoisoindolin-2-yl)piperidine-2,6-dione (C₁₉₂-1)

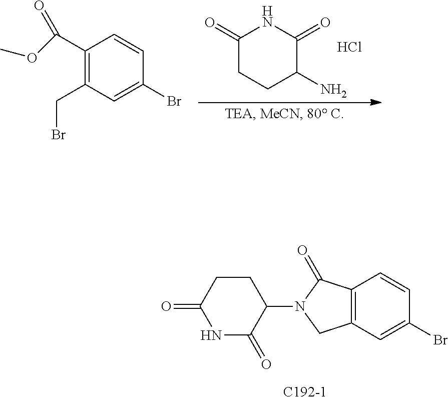 [SMILES: COC(=O)c1ccc(Br)cc1CBr ; NC1CCC(=O)NC1=O ; Cl ; O=C1CCC(N2Cc3cc(Br)ccc3C2=O)C(=O)N1 ; "C192-1"]

Methyl 4-bromo-2-(bromomethyl)benzoate (300 mg, 0.974 mmol), 3-aminopiperidine-2,6-dione hydrochloride (208 mg, 1.266 mmol), and triethylamine (128 mg, 1.266 mmol) were dissolved in 5 mL of acetonitrile, and the mixture was stirred at 80° C. under nitrogen protection overnight. The mixture was cooled to room temperature. The solvent was removed under reduced pressure, and the crude product was purified by Flash to afford white solid compound C192-1 (180 mg, yield 57.0%). LCMS: [M+H]⁺=323, 325.

### Preparation of 4-(7-(4-(1-(4-((5-chloro-4-((2-(dimethylphosphoryl)phenyl)amino)pyrimidin-2-yl)amino)-3-methoxyphenyl)piperidin-4-yl)piperazin-1-yl)-7-oxoheptyl)-2-(2,6-dioxopiperidin-3-yl)isoindoline-1,3-dione (A1)

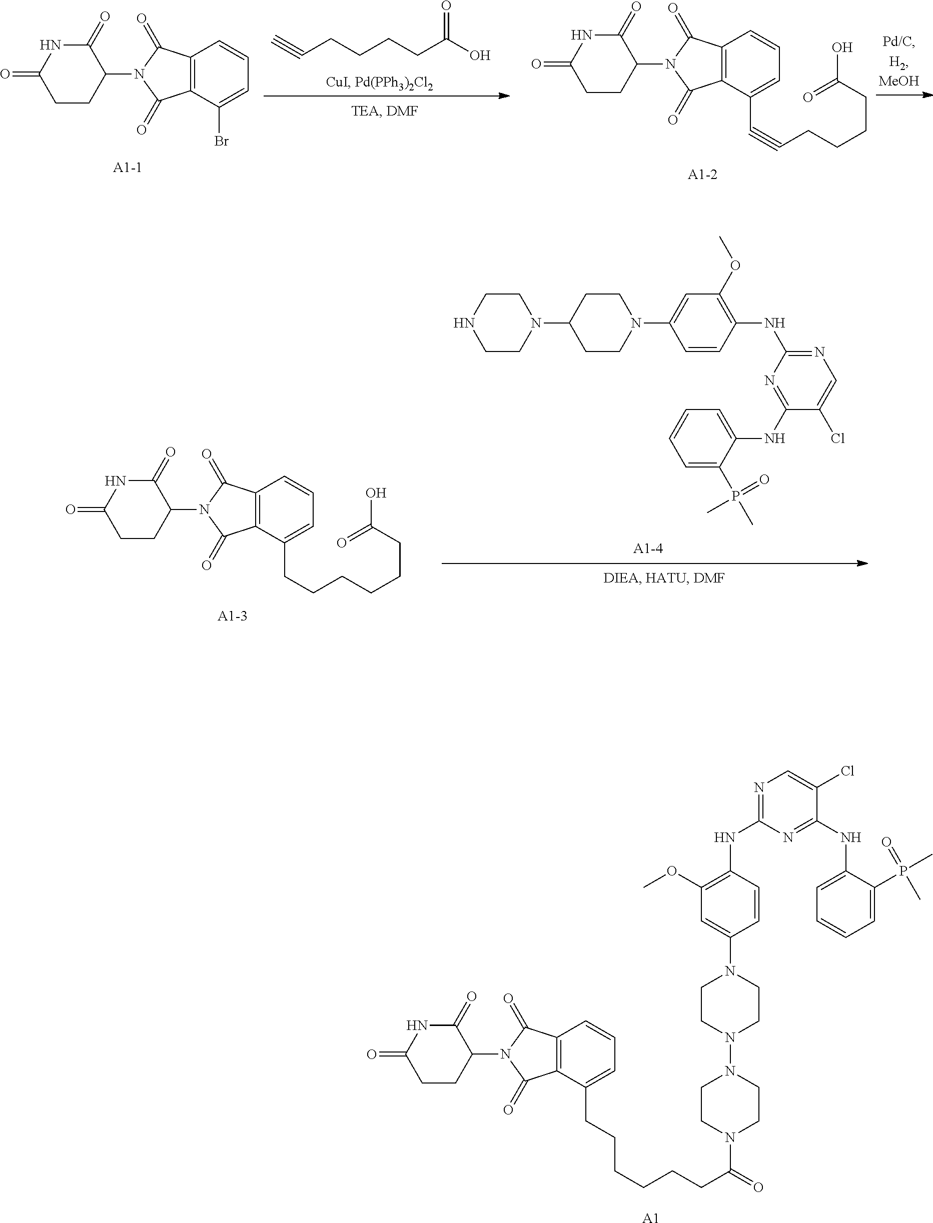

### Step 1

A1-1 (600 mg, 1.79 mmol), 6-alkynylheptanoic acid (450 mg, 3.58 mmol), CuI (69 mg, 0.36 mmol), Pd(PPh₃)₂Cl₂ (501 mg, 0.71 mmol) and triethylamine (903 mg, 8.94 mmol) were added to DMF (15 mL), and the mixture was stirred at 70° C. under nitrogen protection for 5 h. TLC showed that the raw materials were consumed and LCMS showed that the title product was produced. The mixture was concentrated under reduced pressure, and the crude product was separated by column chromatography to afford solid A1-2 (680 mg, yield: 99.7%). LCMS: [M+H]+=383.

### Step 2

A1-2 (340 mg, 0.89 mmol) and Pd/C (10%, 74 mg) were added to methanol (8 mL), and the mixture was hydrogenated at room temperature at 3 MPa for 7 h. The mixture was filtered to remove solids, and the solvent was removed under reduced pressure to afford solid A1-3 (300 mg, yield: 87.5%). LCMS: [M+H]+=387.

### Step 3

A1-3 (85 mg, 0.22 mmol), DIEA (85 mg, 0.66 mmol) and HATU (84 mg, 0.22 mmol) were dissolved in DMF, and the mixture was stirred at room temperature under nitrogen protection for 0.5 h. Then, A1-4 (175 mg, 0.31 mmol) was added and the mixture was further stirred for 3 h. TLC showed that the raw materials were consumed and LCMS showed that the title product was produced. The mixture was directly purified by Flash chromatography to afford white solid A1 (50 mg, yield: 24.3%). LCMS: [M+H]+=938.

### Preparation of 4-((7-(4-(1-(4-((5-chloro-4-((2-(dimethylphosphoryl)phenyl)amino)pyrimidin-2-yl)amino)-3-methoxyphenyl)piperidin-4-yl)piperazin-1-yl)-7-oxoheptyl)oxy)-2-(2,6-dioxopiperidin-3-yl)isoindoline-1,3-dione (A2)

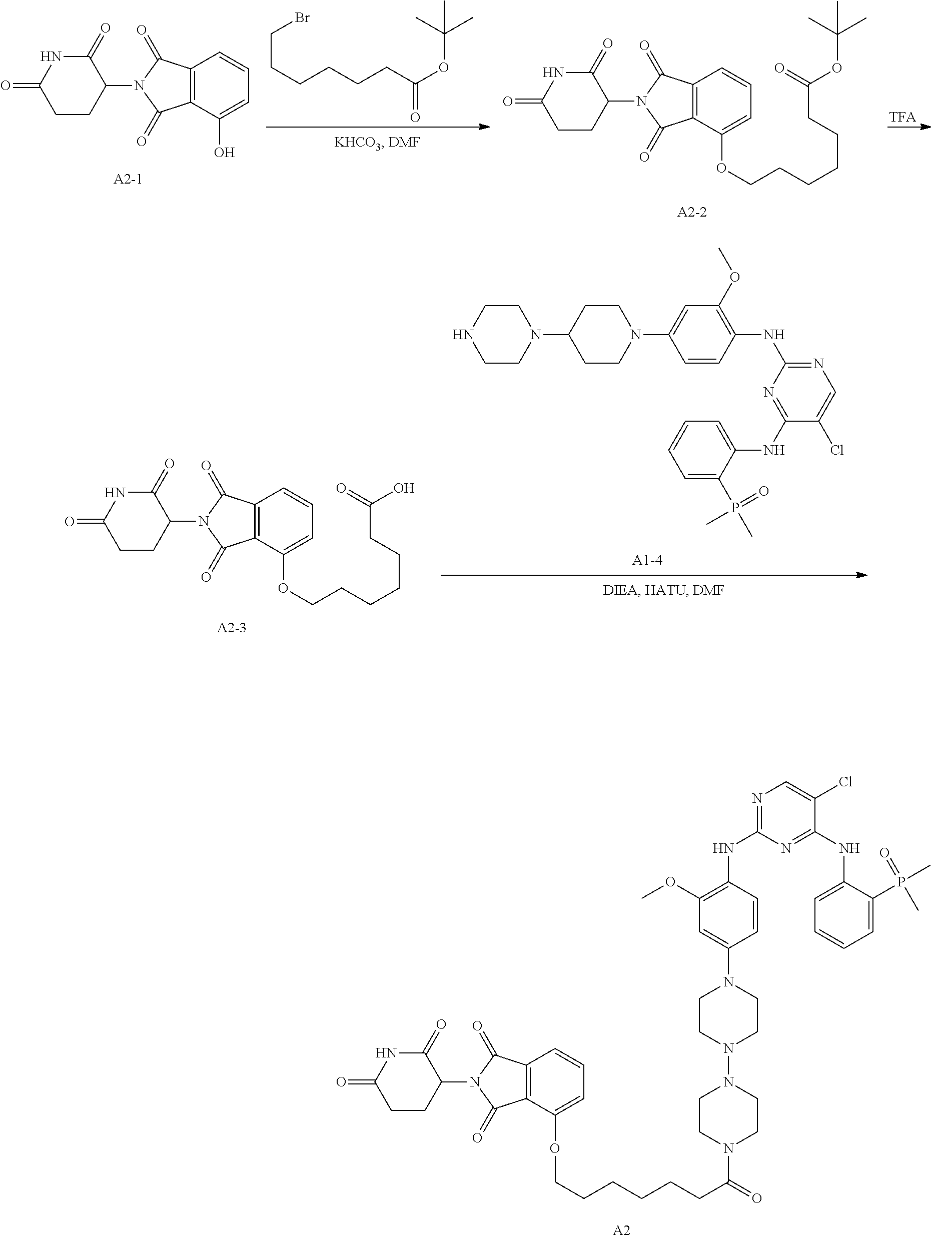

### Step 1

A2-1 (200 mg, 0.73 mmol), tert-butyl 7-bromoheptanoate (232 mg, 0.87 mmol), KI (12 mg, 0.072 mmol) and potassium bicarbonate (110 mg) were dispersed in DMF (5 mL), and the mixture was stirred at 60° C. under nitrogen protection overnight. The mixture was filtered. The solvent was removed under reduced pressure, and the residue was purified by Flash chromatography to afford 160 mg of white solid A2-2.

### Step 2

A2-2 (160 mg) was dissolved in 2 mL of TFA, and the mixture was stirred at room temperature for 1 h. The solvent was removed under reduced pressure to afford crude A2-3, which was directly used in the next step.

### Step 3

A2-3 (140 mg, 0.348 mmol), DIEA (225 mg, 1.744 mmol), and HATU (133 mg, 0.35 mmol) were dissolved in DMF (7 mL), and the mixture was stirred at room temperature under nitrogen protection for 0.5 h. Then, A1-4 (237 mg, 0.417 mmol) was added and the mixture was further stirred for 2 h. The mixture was directly purified by Flash chromatography to afford A2 (25 mg, 7.5%). LCMS: [M+H]⁺=954.

### Preparation of N-(2-(2-(2-(2-(4-(1-(4-((5-chloro-4-((2-(dimethylphosphoryl)phenyl)amino)pyrimidin-2-yl)amino)-3-methoxyphenyl)piperidin-4-yl)piperazin-1-yl)ethoxy)ethoxy)ethoxy)ethyl)-2-((2-(2,6-dioxopiperidin-3-yl)-1,3-dioxoisoindolin-4-yl)oxy)acetamide (A3)

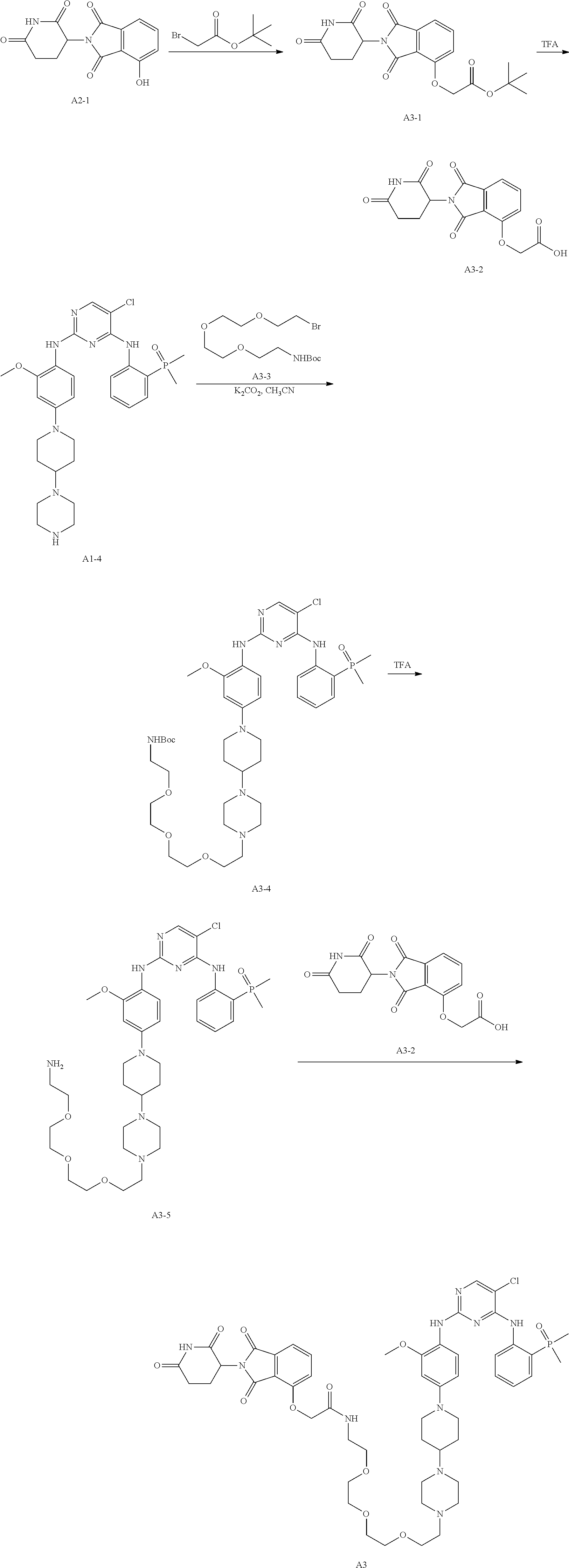

### Step 1

A2-1 (300 mg, 1.15 mmol), tert-butyl bromoacetate (467 mg, 1.79 mmol), KI (18 mg, 0.109 mmol) and potassium bicarbonate (164 mg, 1.64 mmol) were dispersed in DMF, and the mixture was stirred at 60° C. under nitrogen protection overnight. The mixture was filtered. The solvent was removed under reduced pressure, and the residue was purified by Flash chromatography to afford 116 mg of product A3-1. Yield: 26.9%.

### Step 2

A3-1 (116 mg) was dissolved in TFA (1 mL), and the mixture was stirred at room temperature for 1 h. The solvent was evaporated to afford crude A3-2, which was directly used in the next step.

### Step 3

Under nitrogen protection, A1-4 (150 mg, 0.21 mmol), A3-3 (94 mg, 0.26 mmol), and K₂CO₃ (73 mg, 0.53 mg) were added to acetonitrile, and the mixture was stirred at 90° C. overnight. The mixture was filtered to remove solids. The solvent was removed under reduced pressure, and the residue was purified by Flash chromatography to afford compound A3-4 (110 mg, yield: 49.5%).

### Step 4

A3-4 (110 mg, 0.13 mmol) and TFA (1 mL) were dissolved in dichloromethane, and the mixture was stirred at room temperature for 1 h. The solvent was removed under reduced pressure to afford crude A3-5, which was directly used in the next step.

### Step 5

A3-2 (99 mg, 0.31 mmol), DIEA (192 mg, 1.49 mmol), and HATU (118 mg, 0.31 mmol) were dissolved in DMF, and the mixture was stirred at room temperature under nitrogen protection for 0.5 h. Then, A3-5 (222 mg, 0.30 mmol) was added and the mixture was further stirred for 3 h. The mixture was directly purified by Flash chromatography to afford white solid A3 (25 mg, yield:18.1%). LCMS: [M+H]⁺=1059.

### Preparation of 2-(4-(1-(4-((5-chloro-4-((2-(dimethylphosphoryl)phenyl)amino)pyrimidin-2-yl)amino)-3-methoxyphenyl)piperidin-4-yl)piperazin-1-yl)-N-(2-((2-(2,6-dioxopiperidin-3-yl)-1,3-dioxoisoindolin-4-yl)amino)ethyl)acetamide (A6)

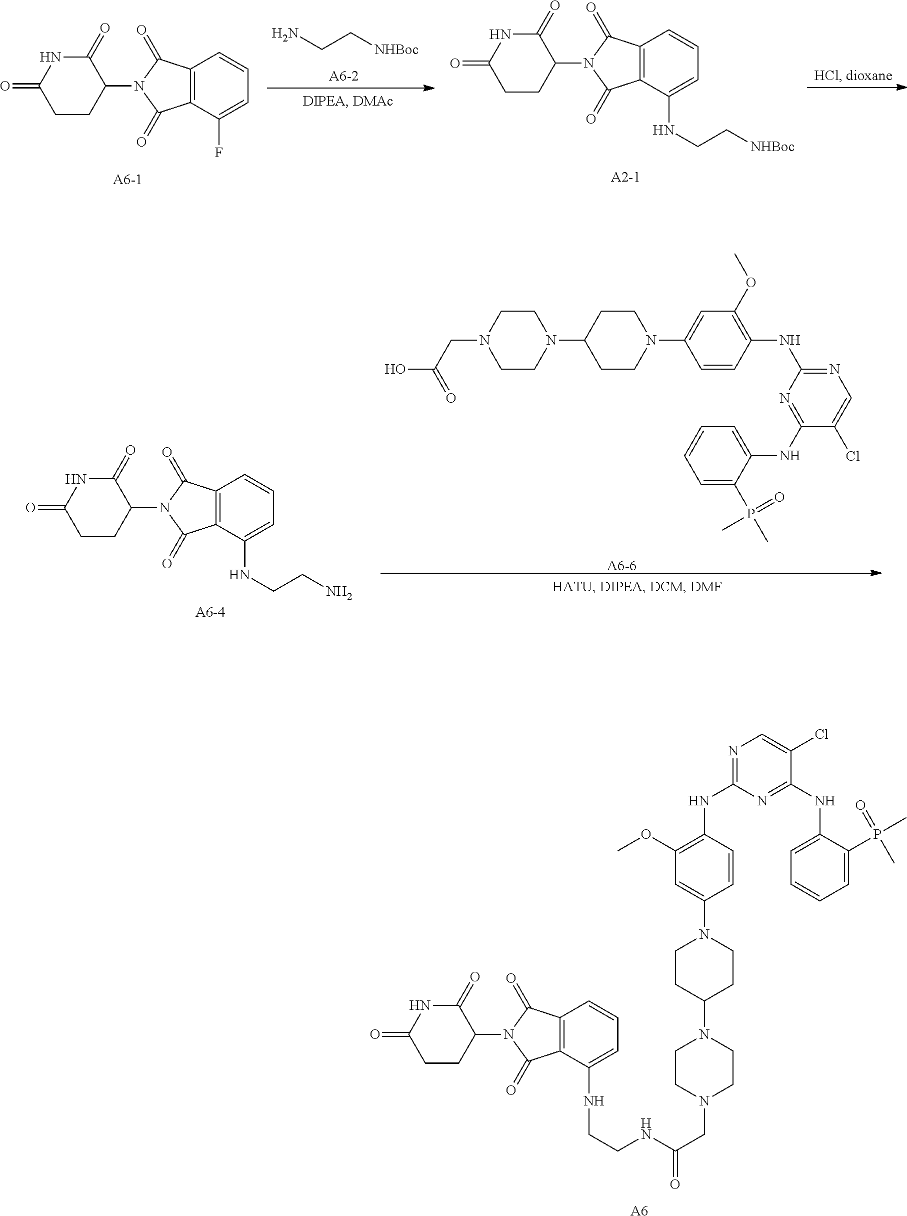

### Step 1

Compound A6-1 (276.2 mg, 1.0 mmol), compound A6-2 (160.2 mg, 1.0 mmol), DIPEA (387 mg, 3.0 mmol) and N,N-dimethylacetamide (5 mL) were added successively to a three-necked flask equipped with a condenser. The mixture was heated to 90° C. and reacted with stirring under nitrogen protection overnight. LCMS showed that the raw materials were basically consumed, and the title product was produced. The mixture was cooled, then diluted with 50 mL of water, and extracted with ethyl acetate for three times (25 mL for each time). The organic phases were combined, and washed with saturated brine for three times (20 mL for each time). The organic phase was dried with anhydrous sodium sulfate for 2 h, and filtered. The filtrate was concentrated under reduced pressure to afford a crude product, which was separated by silica gel column chromatography to afford yellow solid A6-3 (200 mg, yield: 48%).

### Step 2

Compound A6-3 (100 mg, 0.24 mmol) and 1,4-dioxane (5 mL) were added to a 50 mL single-necked flask and dissolved with with stirring. HCl/1,4-dioxane solution (2 mL, 4M) was added, and the mixture was reacted with stirring at room temperature for 2 h. TLC showed that the raw materials were consumed and LCMS showed that the title product was produced. The mixture was concentrated under reduced pressure to afford a hydrochloride of compound A6-4, which was yellow solid (80 mg, yield: 95%). LCMS: [M+H]⁺=317.

### Step 3

Compound A6-5 (31.4 mg, 0.05 mmol), HATU (23 mg, 0.06 mmol) and dichloromethane (3 mL) were added to a 50 mL three-necked flask. The mixture was dissolved with stirring at room temperature under nitrogen protection. DIPEA (64 mg, 0.5 mmol) was then added, and the mixture was reacted with stirring at room temperature for 30 minutes. Then, a solution of compound A6-4 (22 mg, 0.05 mmol) in DMF (2.0 mL) was added. The mixture was stirred at room temperature overnight. LCMS showed that the title product was produced. The mixture was concentrated under reduced pressure to remove dichloromethane, and separated by Flash preparative column chromatography to afford a light-yellow powder, which was compound A6 (14 mg, yield: 30.4%). LCMS: [M+H]⁺=926.

### Preparation of 2-(4-(1-(4-((5-chloro-4-((2-(dimethylphosphoryl)phenyl)amino)pyrimidin-2-yl)amino)-3-methoxyphenyl)piperidin-4-yl)piperazin-1-yl)-N-(2-(2-(2-(2-((2-(2,6-dioxopiperidin-3-yl)-1,3-dioxoisoindolin-4-yl)amino)ethoxy)ethoxy)ethoxy)ethyl)acetamide (A8)

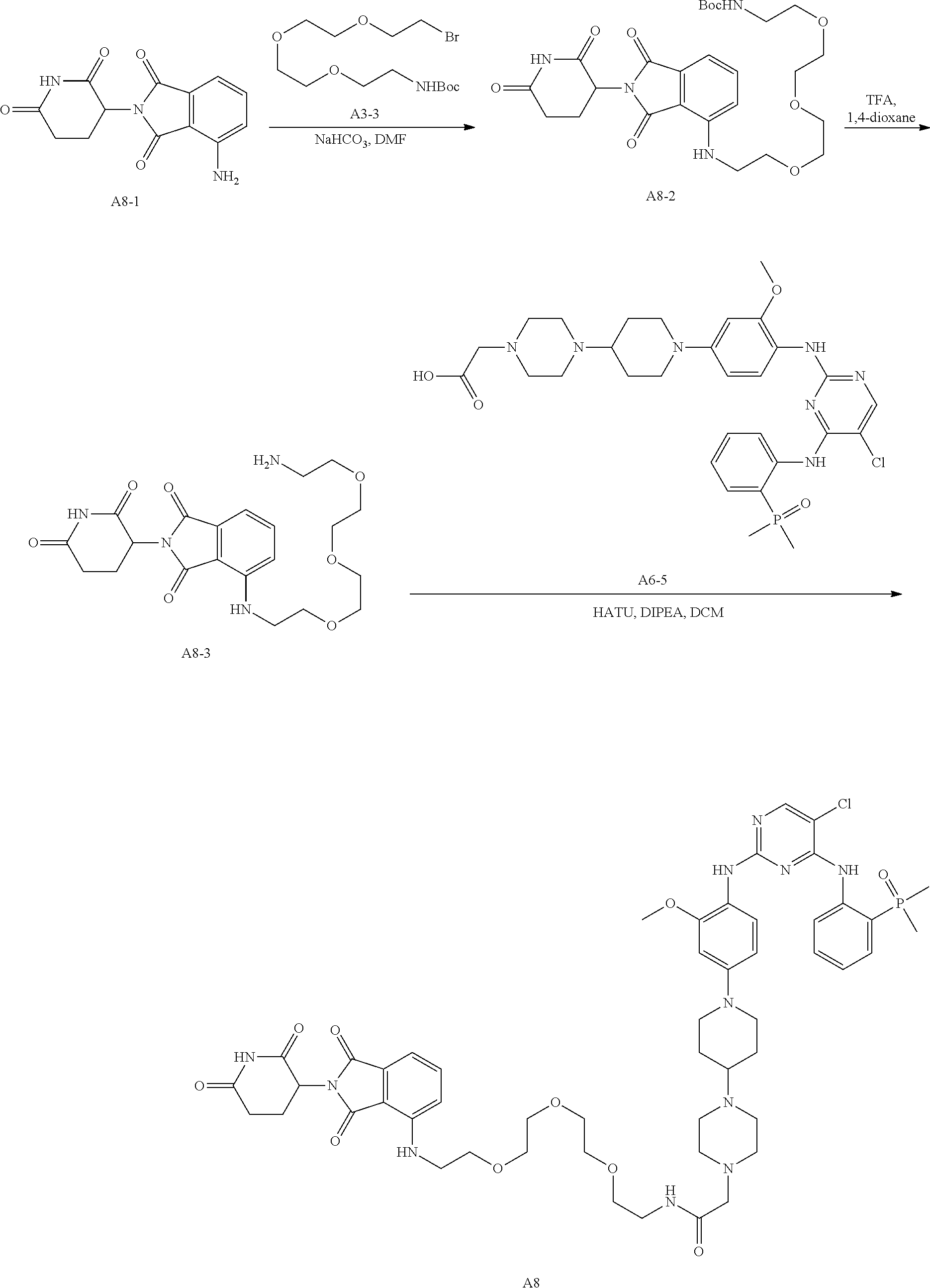

### Step 1

Compound A3-3 (260.7 mg, 0.732 mmol), commercially purchased compound A8-1 (200 mg, 0.732 mmol), sodium bicarbonate (232 mg, 2.196 mmol) and N,N-dimethylformamide (5 mL) were added successively to a 20 mL microwave reaction tube, and the mixture was heated to 90° C. in a microwave reactor under nitrogen protection for 2 h (reactor: CEM, power: 100 W). Thin layer chromatography showed that compound A8-1 basically disappeared, and LCMS showed that the title product was produced. The mixture was cooled and filtered to remove inorganic salts, diluted by adding 20 mL of water, and extracted with ethyl acetate for three times (25 mL for each time). The organic phases were combined, washed twice with saturated brine (10 mL for each time), then dried with anhydrous sodium sulfate, and filtered. The filtrate was concentrated under reduced pressure to afford a crude product, which was separated by silica gel column chromatography to afford a compound A8-2 (100 mg, yield: 24.9%).

### Step 2

Compound A8-2 (100 mg, 0.182 mmol) and 1,4-dioxane (4 mL) were added to a 50 mL single-necked flask and dissolved with stirring. Trifluoroacetate (2 mL) was then added, and the mixture was reacted with stirring at room temperature for 2 h. TLC showed that the reaction was completed. The mixture was concentrated under reduced pressure to afford brown oily product. The brown oily product was dissolved with 20 mL of dichloromethane, washed twice with saturated sodium bicarbonate solution (5 mL for each time) and once with 10 mL of water, dried with anhydrous sodium sulfate, and filtered. The filtrate was concentrated under reduced pressure to afford 55 mg of yellow green solid A8-3. Yield: 68%. LCMS: [M+H]+=449.

### Step 3

Compound A6-5 (70 mg, 0.1 mmol), HATU (45.6 mg, 0.12 mmol) and dichloromethane (3 mL) were added to a 50 mL three-necked flask. The mixture was dissolved with stirring at room temperature under nitrogen protection. DIPEA (64.5 mg, 0.5 mmol) was then added, and the mixture was reacted with stirring at room temperature for 30 minutes. Then compound A8-3 (45 mg, 0.1 mmol) was added, and the mixture was stirred at room temperature overnight. LCMS showed that the title product was produced. The mixture was diluted with 20 mL of dichloromethane, washed twice with water (5 mL for each time), and concentrated under reduced pressure to afford a crude product. The crude product was separated by a silica gel preparative plate to afford light-yellow powder A8 (22 mg, yield: 20.8%). LCMS: [M+H]+=1058.

### Preparation of 4-((7-(4-(1-(4-((5-chloro-4-((2-(dimethylphosphoryl)phenyl)amino)pyrimidin-2-yl)amino)-3-methoxyphenyl)piperidin-4-yl)piperazin-1-yl)heptyl)oxy)-2-(2,6-dioxopiperidin-3-yl)isoindoline-1,3-dione (A10)

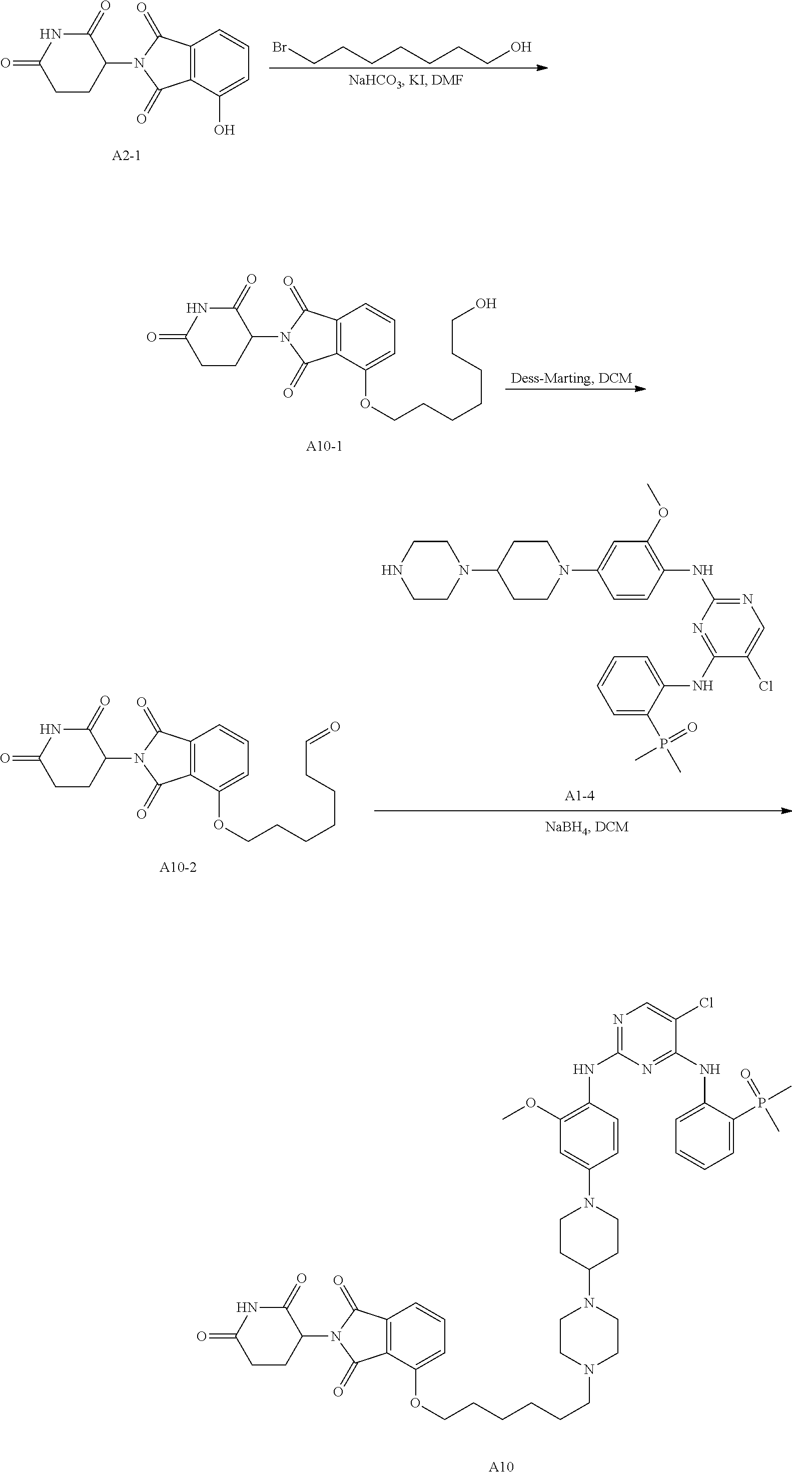

### Step 1

A2-1 (274 mg, 1.0 mmol), NaHCO₃ (252 mg, 3.0 mmol) and KI (83 mg, 0.5 mmol) were placed in a 20 mL reaction flask. 5.5 mL of anhydrous DMF was added under nitrogen protection, and 7-bromo-1-heptanol (292.5 mg, 1.5 mmol) was added dropwise with stirring. After the dropwise addition, the mixture was reacted at 70° C. for 20 h. The mixture was diluted with 100 mL of ethyl acetate, and then washed successively with saturated NH₄Cl solution (30 mL×1), H₂O (30 mL×3) and saturated brine (30 mL×2). The organic layer was dried with anhydrous Na₂SO₄, and filtered with suction to remove the desiccant. The filtrate was concentrated under reduced pressure to afford a crude product, which was purified by Prep-TLC to afford 300 mg of white solid product A10-1. Yield: 77.3%. LCMS: [M+H]⁺=389. ¹H-NMR: (400 MHz, DMSO-d₆) δ 11.092 (s, 1H), 7.827 (dd, J=7.6 Hz, 8.8 Hz, 1H), 7.523 (dd, J=8.4 Hz, 29.6 Hz, 2H), 5.098-5.052 (m, 1H), 4.358 (t, J=5.2 Hz, 1H), 4.218 (t, J=6.4 Hz, 2H), 2.926-2.836 (m, 1H), 2.045-1.995 (m, 1H), 1.791-1.722 (m, 2H), 1.492-1.237 (m, 12H).

### Step 2

A10-1 (80 mg, 0.206 mmol) was dissolved in 15 mL of anhydrous DCM, and Dess-Martin reagent (262 mg, 0.618 mmol) was added under nitrogen protection. After the addition, the mixture was reacted at 55° C. for 2 h. The mixture was diluted with 30 mL of DCM, and then 15 mL of saturated NaHCO₃ solution and 15 mL of saturated Na₂S₂O₃ solution were added. The mixture was stirred for 5 min. The organic layer was separated, dried with anhydrous Na₂SO₄, and filtered with suction to remove the desiccant. The filtrate was concentrated under reduced pressure to afford crude A10-2 (75 mg). LCMS: [M+H]⁺=387. The crude product was directly used in the next step.

### Step 3

A10-2 (75 mg, 0.194 mmol) and A1-4 (110.3 mg, 0.194 mmol) were dissolved in 12.0 mL of anhydrous DCM. 2 drops of CH₃COOH was added under nitrogen protection. The mixture was then stirred at room temperature for 5 min. NaBH₄ (73.7 mg, 1.94 mmol) was then added, and the mixture was reacted with stirring at room temperature for 2 h. The mixture was diluted with 30 mL of DCM, and then 15 mL saturated NH₄Cl solution was added and stirred for 5 min. The organic layer was separated, washed once with 15 mL of saturated brine, then dried with anhydrous Na₂SO₄, and filtered with suction to remove the desiccant. The filtrate was concentrated under reduced pressure to afford a crude product. The crude product was purified by RP-Flash chromatography to afford 65 mg of white solid with a purity of about 60%. The product was further purified by Prep-TLC to afford 30 mg of white solid product with a purity of 90%. The product was purified again by RP-Flash chromatography to afford 15 mg of white solid pure product. Yield: 8.2%. LCMS: [M+H]+=940.5. ¹H-NMR: (400 MHz, DMSO-d₆) δ 11.164 (s, 1H), 11.106 (s, 1H), 8.480 (s, 1H), 8.063-8.046 (m, 2H), 7.827 (t, J=8.0 Hz, 1H), 7.555-7.502 (m, 2H), 7.450-7.432 (m, 1H), 7.391-7.336 (m, 2H), 7.111 (t, J=7.2 Hz, 1H), 6.625 (d, J=2.4 Hz, 1H), 6.474 (d, J=8.0 Hz, 1H), 5.085 (dd, J=5.6, 12.8 Hz, 1H), 4.204 (t, J=6.4 Hz, 2H), 3.757 (s, 3H), 3.724-3.695 (m, 2H), 2.884-2.851 (m, 1H), 2.688-2.661 (m, 2H), 2.630-2.566 (m, 3H), 2.333-2.246 (m, 6H), 2.227-1.990 (m, 2H), 1.858-1.830 (m, 2H), 1.779-1.745 (m, 9H), 1.524-1.239 (m, 12H).

### Preparation of 4-((9-(4-(1-(4-((5-chloro-4-((2-(dimethylphosphoryl)phenyl)amino)pyrimidin-2-yl)amino)-3-methoxyphenyl)piperidin-4-yl)piperazin-1-yl)nonyl)oxy)-2-(2,6-dioxopiperidin-3-yl)isoindoline-1,3-dione (A11)

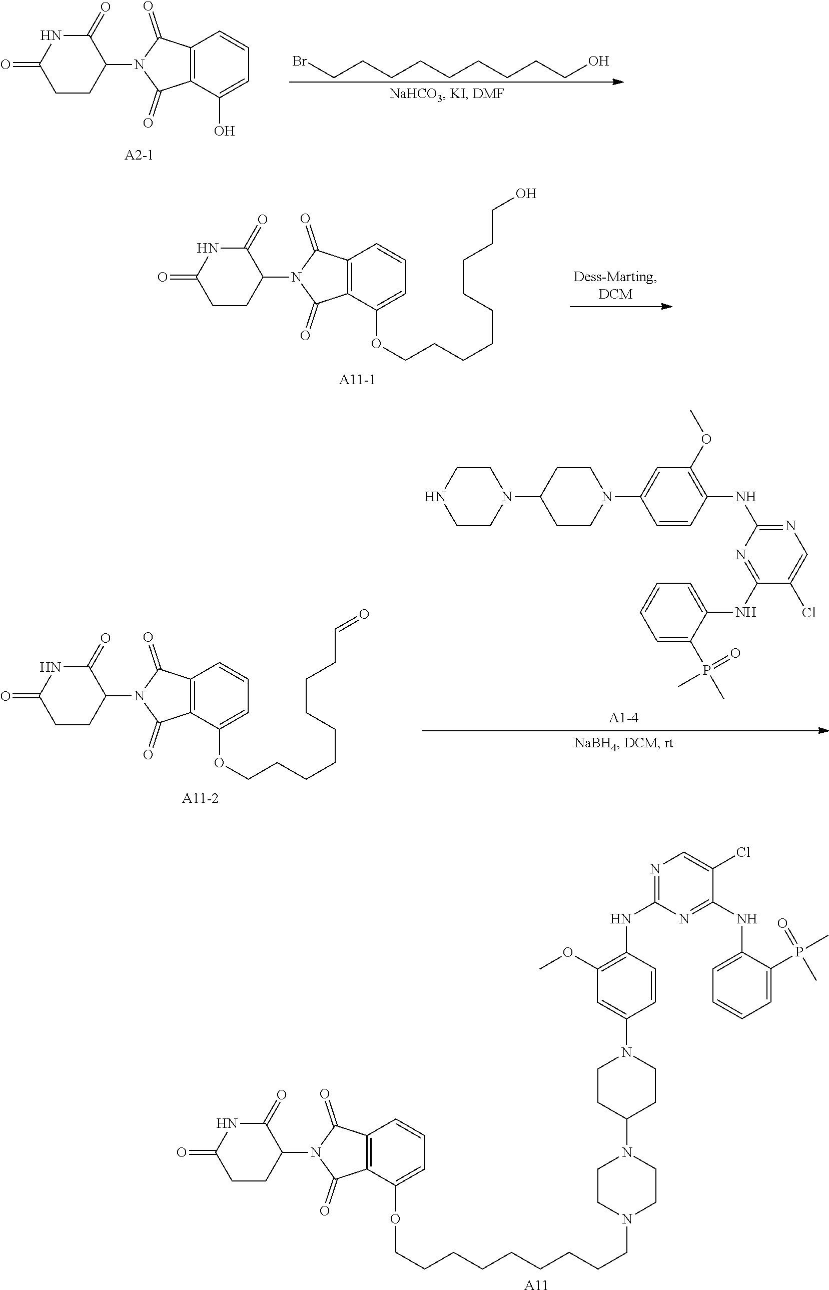

### Step 1

A2-1 (137 mg, 0.5 mmol), NaHCO₃ (126 mg, 1.5 mmol) and KI (41.5 mg, 0.25 mmol) were placed in a 20 mL reaction flask, and 5.0 mL of anhydrous DMF was added under the protection of nitrogen. 9-bromo-1-nonanol (167.3 mg, 0.75 mmol) was added dropwise with stirring. After the dropwise addition was completed, the mixture was reacted at 70° C. for 20 h. The mixture was diluted with 80 mL of ethyl acetate and then washed successively with saturated NH₄Cl solution (20 mL×1), H₂O (20 mL×3) and saturated brine (20 mL×2). The organic layer was dried with anhydrous Na₂SO₄, and filtered with suction to remove the desiccant. The filtrate was concentrated under reduced pressure to afford a crude product, which was purified by Prep-TLC to afford 70 mg of white solid product A11-1. Yield: 33.6%. LCMS: [M+H]+=417.

### Step 2

A11-1 (60 mg, 0.144 mmol) was dissolved in 12 mL of anhydrous DCM, and Dess-Martin reagent (305.8 mg, 0.721 mmol) was added under nitrogen protection. After the addition was completed, the mixture was reacted at 55° C. for 3 h. The mixture was diluted with 30 mL of DCM, and then 15 mL of saturated NaHCO₃ solution and 15 mL of saturated Na₂S₂O₃ solution were added and stirred for 5 min. The organic layer was separated, dried with anhydrous Na₂SO₄, and filtered with suction to remove the desiccant. The filtrate was concentrated under reduced pressure to afford crude A11-2 (55 mg). LCMS: [M+H]⁺=415. The crude product was directly used in the next step.

### Step 3

A11-2 (50 mg, 0.121 mmol) and A1-4 (68.7 mg, 0.121 mmol) were dissolved in 10.0 mL of anhydrous DCM. 2 drops of CH₃COOH was added under nitrogen protection. The mixture was then stirred at room temperature for 5 min, and NaBH₄ (46 mg, 1.21 mmol) was then added. The mixture was reacted with stirring at room temperature for 2 h. The mixture was diluted with 20 mL of DCM, and then 15 mL of saturated NH₄Cl solution was added and stirred for 5 min. The organic layer was separated, washed once with 15 mL of saturated brine, then dried with anhydrous Na₂SO₄, and filtered with suction to remove the desiccant. The filtrate was concentrated under reduced pressure to afford a crude product, which was purified by RP-Flash chromatography to afford 20 mg of white solid product. Yield: 17%. LCMS: [M+H]⁺=968.5. ¹H-NMR: (400 MHz, DMSO-d₆) δ 11.169 (s, 1H), 11.114 (s, 1H), 8.475 (s, 1H), 8.047 (d, J=6.4 Hz, 2H), 7.825-7.860 (m, 1H), 7.555-7.502 (m, 3H), 7.448-7.317 (m, 2H), 7.092 (t, J=7.6 Hz, 1H), 6.624-6.450 (m, 2H), 5.085 (dd, J=5.2, 12.8 Hz, 1H), 4.201 (t, J=6.4 Hz, 2H), 3.725 (t, J=12.8 Hz, 6H), 3.287 (s, 2H), 2.688-2.540 (m, 6H), 2.328-2.195 (m, 6H), 2.029-1.991 (m, 2H), 1.861-1.830 (m, 2H), 1.779-1.746 (m, 10H), 1.661-1.274 (m, 4H), 1.275-1.237 (m, 8H).

### Preparation of 3-(4-((7-(4-(1-(4-((5-chloro-4-((2-(dimethylphosphoryl)phenyl)amino)pyrimidin-2-yl)amino)-3-methoxyphenyl)piperidin-4-yl)piperazin-1-yl)heptyl)amino)-1-oxoisoindolin-2-yl)piperidine-2,6-dione (A12)

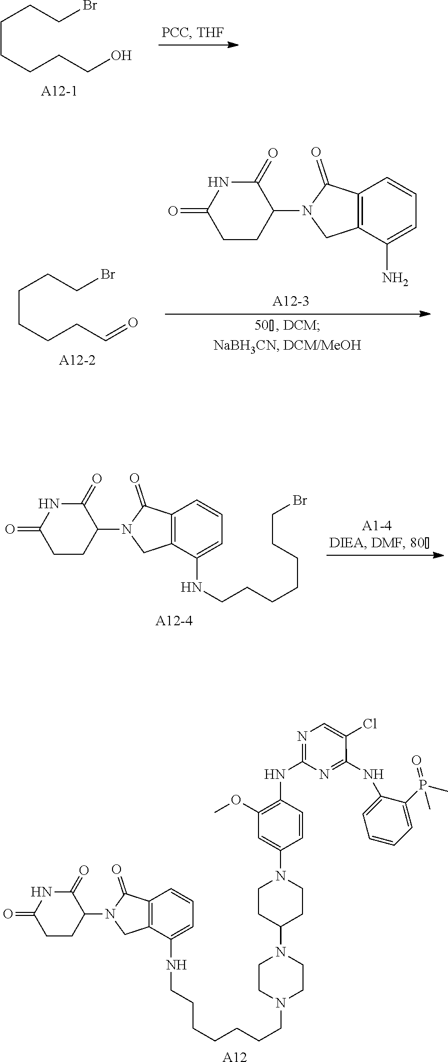

### Step 1

7-Bromo-1-heptanol (200 mg, 1.03 mmol) was added to a solution of pyridinium chlorochromate (333 mg, 1.54 mmol) in THF (5 mL). The mixture was reacted with stirring at room temperature overnight. The solvent was removed under reduced pressure, and the residue was dissolved by adding 20 mL of diethyl ether. The mixture was filtered, and concentrated to afford crude A12-2 (160 mg, yield: 80.8%). LCMS: [M+H]⁺=193, 195.

### Step 2

A12-2 (200 mg, 1.04 mmol) and 3-(4-amino-1-oxoisoindolin-2-yl)piperidine-2,6-dione (A12-3)(270 mg, 1.04 mmol) were dissolved in DCM (10 mL) and MeOH (5 mL). The mixture was stirred at 50° C. under nitrogen protection for 1 h. The mixture was cooled to room temperature, and sodium cyanobohydride (98 mg, 1.56 mmol) and 2 drops of glacial acetic acid were added. The mixture was heated to 50° C. and further stirred for 1.5 h. The mixture was cooled to room temperature, quenched with aqueous saturated NH₄Cl solution, extracted with dichloromethane, and layered. The organic phases were combined, dried with anhydrous sodium sulfate, allowed to stand, and filtered. The filtrate was concentrated, and the crude product was purified by Flash chromatography to afford A12-4 (100 mg, yield: 22.1%). LCMS: [M+H]+=436, 438.

### Step 3

A12-4 (10 mg, 0.023 mmol), A1-4 (13 mg, 0.023 mmol) and DIEA (16 mg, 0.124 mmol) were dissolved in DMF (0.5 mL). The mixture was stirred at 80° C. under nitrogen protection for 7 h. The solvent was removed under reduced pressure, and the residue was purified by Flash chromatography to afford A12 (2.97 mg, yield: 14.0%). LCMS: [M+H]+=925. ¹H NMR (400 MHz, DMSO-d₆) δ 11.17 (s, 1H), 11.00 (s, 1H), 8.48 (s, 1H), 8.06 (d, J=5.3 Hz, 1H), 7.53 (dd, J=13.9, 7.4 Hz, 1H), 7.43-7.24 (m, 4H), 7.18 (s, 1H), 7.09 (t, J=7.4 Hz, 1H), 6.92 (d, J=7.4 Hz, 1H), 6.74 (d, J=8.1 Hz, 1H), 6.62 (d, J=2.5 Hz, 1H), 6.47 (d, J=8.3 Hz, 1H), 5.55 (s, 1H), 5.11 (dd, J=13.1, 5.1 Hz, 1H), 4.23 (d, J=17.0 Hz, 1H), 4.12 (d, J=17.2 Hz, 1H), 3.76 (s, 3H), 3.73 (m, 2H), 3.52 (m, 1H), 3.10 (m, 2H), 2.93 (m, 1H), 2.63 (m, 3H), 2.33 (m, 3H), 2.29-2.27 (m, 2H), 2.03-1.98 (m, 4H), 1.85 (m, 2H), 1.78 (s, 3H), 1.75 (s, 3H), 1.57-1.50 (m, 6H), 1.42 (m, 2H), 1.34 (m, 2H), 1.26 (m, 2H).

### Preparation of 3-(4-((9-(4-(1-(4-((5-chloro-4-((2-(dimethylphosphoryl)phenyl)amino)pyrimidin-2-yl)amino)-3-methoxyphenyl)piperidin-4-yl)piperazin-1-yl)nonyl)amino)-1-oxoisoindolin-2-yl)piperidine-2,6-dione (A13)

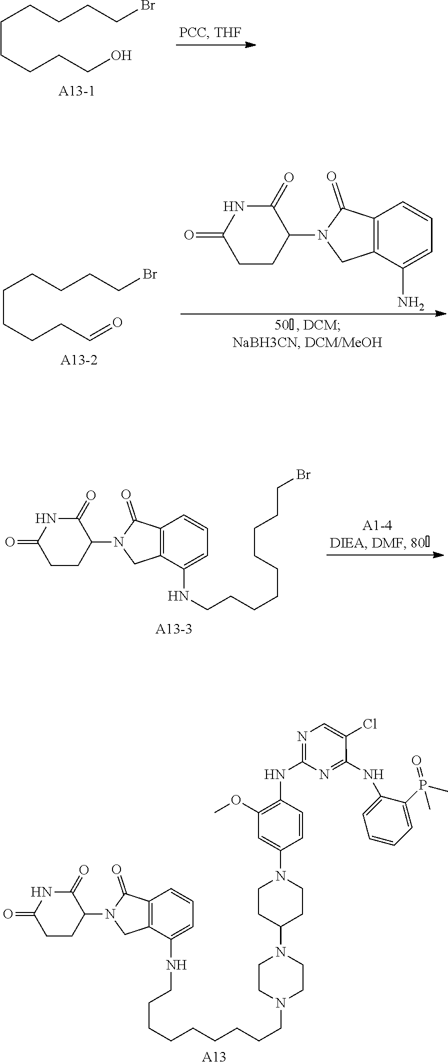

### Step 1

9-Bromo-1-nonanol (222 mg, 1.03 mmol) was added to a solution of pyridinium dichlorochromate (333 mg, 1.54 mmol) in THE (5 mL). The mixture was reacted with stirring at room temperature overnight. The solvent was removed under reduced pressure, and the residue was dissolved by adding 20 mL of diethyl ether. The mixture was filtered, and concentrated to afford crude A13-2 (150 mg, yield: 68.2%). LCMS: [M+H]⁺=221, 223.

### Step 2

A13-2 (228 mg, 1.04 mmol) and 3-(4-amino-1-oxoisoindolin-2-yl)piperidine-2,6-dione (A12-3) (270 mg, 1.04 mmol) were dissolved in DCM (10 mL) and MeOH (5 mL). The mixture was stirred at 50° C. under nitrogen protection for 1 h. The mixture was cooled to room temperature, and sodium cyanobohydride (98 mg, 1.56 mmol) and 2 drops of glacial acetic acid were added. The mixture was heated to 50° C. and further stirred for 1.5 h. The mixture was cooled to room temperature, quenched with aqueous saturated NH₄Cl solution, extracted with dichloromethane, and layered. The organic phases were combined, dried with anhydrous sodium sulfate, allowed to stand, and filtered. The filtrate was concentrated, and the crude product was purified by Flash chromatography to afford A13-3 (100 mg, yield: 20.9%). LCMS: [M+H]+=464, 466.

### Step 3

A13-3 (11 mg, 0.023 mmol), A1-4 (13 mg, 0.023 mmol) and DIEA (16 mg, 0.124 mmol) were dissolved in DMF (0.5 mL). The mixture was stirred at 80° C. under nitrogen protection for 7 h. The solvent was removed under reduced pressure, and the residue was purified by Flash chromatography to afford A13 (2.49 mg, yield: 11.0%). LCMS: [M+H]+=953. ¹H-NMR (400 MHz, DMSO-d₆) δ 11.17 (s, 1H), 11.01 (s, 1H), 8.48 (s, 1H), 8.06 (s, 1H), 7.56-7.50 (m, 1H), 7.40-7.25 (m, 4H), 7.18 (s, 1H), 7.09 (t, J=7.5 Hz, 1H), 6.90 (dd, J=15.3, 7.3 Hz, 1H), 6.77-6.67 (m, 1H), 6.62 (d, J=2.6 Hz, 1H), 6.47 (d, J=9.2 Hz, 1H), 5.54 (t, 1H), 5.11 (dd, J=13.2, 5.0 Hz, 1H), 4.23 (d, J=17.2 Hz, 1H), 4.12 (d, J=17.2 Hz, 1H), 3.76 (s, 3H), 3.73 (m, 1H), 3.70 (m, 1H), 3.52 (s, 1H), 3.11 (d, J=6.1 Hz, 2H), 2.99-2.86 (m, 1H), 2.66 (t, J=11.7 Hz, 3H), 2.32 (m, 3H), 2.26 (m, 2H), 2.01 (m, 4H), 1.85 (d, J=12.2 Hz, 2H), 1.78 (s, 3H), 1.75 (s, 3H), 1.59-1.49 (m, 4H), 1.44-1.38 (m, 4H), 1.34 (m, 2H), 1.30 (m, 2H), 1.27 (m, 2H), 1.26 (m, 2H), 1.25 (m, 2H).

### Preparation of 2-(4-(1-(4-((5-chloro-4-((2-(dimethylphosphoryl)phenyl)amino)pyrimidin-2-yl)amino)-3-methoxyphenyl)piperidin-4-yl)piperazin-1-yl)-N-(2-(2,6-dioxopiperidin-3-yl)-1-oxoisoindolin-4-yl)acetamide (A17)

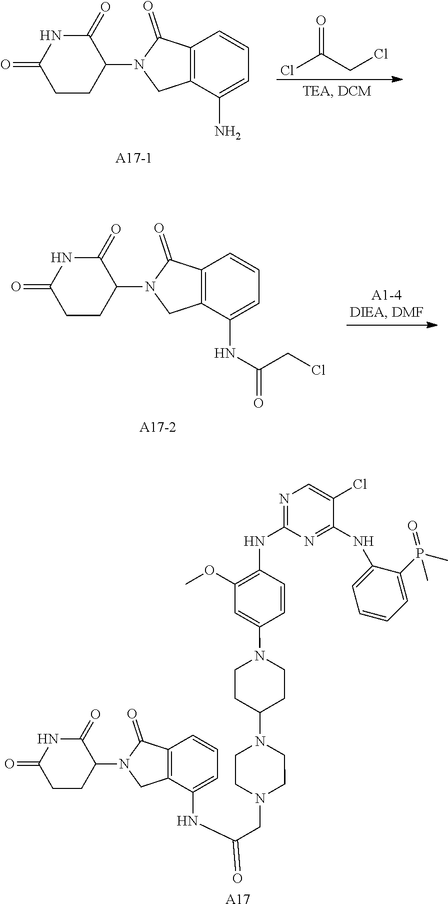

### Step 1

A17-1 (or A12-3) (200 mg, 0.772 mmol) and TEA (234 mg, 2.32 mmol) were dissolved in DCM (3 mL), and chloroacetyl chloride (78 mg, 0.697 mmol) was added dropwise at 0° C. under nitrogen protection. After the dropwise addition, the mixture was further stirred at 0° C. for 2 h. LCMS showed that the reaction was completed. 1 mL of methanol was added to quench the reaction. The solvent was removed at 40° C. under reduced pressure, and the crude product was purified by Flash chromatography to afford white solid A17-2 (170 mg, yield: 73.0%). LCMS: [M+H]⁺=336.

### Step 2

A17-2 (30 mg, 0.090 mmol), A1-4 (51 mg, 0.090 mmol) and DIEA (59 mg, 0.457 mmol) were added to DMF (2 mL). The mixture was reacted with stirring at 60° C. under nitrogen protection for 2 h. LCMS showed that the reaction was completed. The mixture was purified by Flash chromatography to afford off-white solid A17 (15 mg, yield: 19.5%). LCMS: [M+H]+=869. ¹H-NMR (400 MHz, DMSO-d₆) δ 11.17 (s, 1H), 11.03 (s, 1H), 8.49 (s, 1H), 8.06 (m, J=4.4 Hz, 2H), 7.87-7.72 (m, 1H), 7.58-7.49 (m, 3H), 7.38 (dd, J=15.7, 8.0 Hz, 1H), 7.10 (t, J=7.6 Hz, 1H), 6.71-6.60 (m, 1H), 6.56-6.45 (m, 1H), 5.15 (dd, J=13.3, 5.2 Hz, 2H), 4.45-4.33 (m, 3H), 3.97-3.89 (m, 1H), 3.80-3.69 (m, 4H), 3.49 (d, J=15.9 Hz, 1H), 3.38 (s, 1H), 3.19 (s, 1H), 3.02 (s, 4H), 2.65 (q, J=17.0, 15.0 Hz, 8H), 2.42-2.22 (m, 2H), 1.90 (d, J=12.9 Hz, 3H), 1.78 (s, 3H), 1.75 (s, 2H), 1.28 (d, J=15.9 Hz, 2H).

### Preparation of 3-(4-((2-(4-(1-(4-((5-chloro-4-((2-(dimethylphosphoryl)phenyl)amino)pyrimidin-2-yl)amino)-3-methoxyphenyl)piperidin-4-yl)piperazin-1-yl)-2-oxoethyl)amino)-1-oxoisoindolin-2-yl)piperidine-2,6-dione (A18)

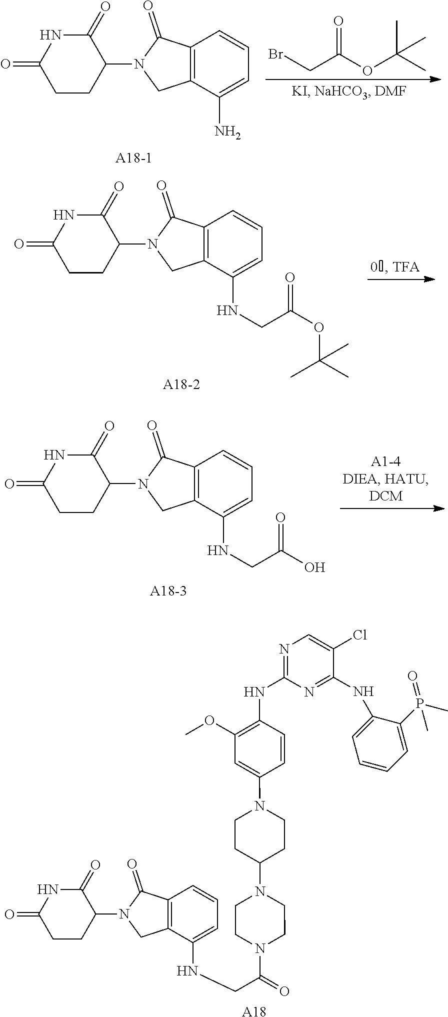

### Step 1

A18-1 (or A12-3) (259.1 mg, 1 mmol), 2-tert-butyl bromoacetate (232.8 mg, 1.2 mmol), KI (16.6 mg, 0.1 mmol) and NaHCO₃ (126.0 mg, 1.5 mmol) were added to DMF (7 mL). The mixture was stirred at 60° C. under nitrogen protection overnight. The solvent was removed under reduced pressure. The crude product was separated by column chromatography to afford white solid compound A18-2 (150 mg, yield: 40.2%).

### Step 2

At 0° C., A18-2 (44 mg, 0.118 mmol) was dissolved in 1 mL of TFA. The mixture was stirred at room temperature for 1 h. The solvent was removed at 40° C. under reduced pressure to afford crude A18-3. LCMS: [M+H]⁺=318.

### Step 3

The above crude A18-3 was dispersed in dichloromethane (2 mL) at room temperature, and DIEA (81 mg, 0.628 mmol) and HATU (44 mg, 0.116 mmol) were added. The mixture was stirred at room temperature under nitrogen protection for 0.5 h. A1-4 (51 mg, 0.090 mmol) was then added and further stirred at room temperature for 3 h. LCMS showed that the reaction was completed. The solvent was removed at 40° C. under reduced pressure. The crude product was purified by Flash chromatography to afford off-white solid A18 (25 mg, yield: 32.1%). LCMS: [M+H]+=869. ¹H-NMR (400 MHz, DMSO-d₆) δ 11.16 (s, 1H), 8.48 (s, 1H), 8.05 (d, J=7.4 Hz, 2H), 7.56-7.48 (m, 1H), 7.40-7.33 (m, 3H), 7.19 (t, J=7.6 Hz, 1H), 7.09 (t, J=7.4 Hz, 1H), 6.92 (d, J=7.4 Hz, 1H), 6.80 (d, J=7.9 Hz, 1H), 6.68-6.60 (m, 1H), 6.52-6.44 (m, 1H), 5.45 (s, 1H), 5.27 (dd, J=13.3, 5.2 Hz, 1H), 4.51 (d, J=6.1 Hz, 3H), 4.24 (d, J=16.9 Hz, 1H), 4.02 (d, J=16.8 Hz, 1H), 3.75 (s, 4H), 3.46 (d, J=26.8 Hz, 3H), 3.09 (s, 1H), 2.84 (d, J=17.2 Hz, 1H), 2.66 (t, J=12.1 Hz, 8H), 2.30 (d, J=13.6 Hz, 1H), 2.15 (s, 1H), 1.84 (d, J=11.8 Hz, 1H), 1.76 (d, J=13.5 Hz, 6H), 1.55 (d, J=11.8 Hz, 1H), 1.28 (d, J=15.9 Hz, 2H), 0.85 (t, J=6.6 Hz, 1H).

### Preparation of 3-(4-(7-(4-(1-(4-((5-chloro-4-((2-(dimethylphosphoryl)phenyl)amino)pyrimidin-2-yl)amino)-3-methoxyphenyl)piperidin-4-yl)piperazin-1-yl)hept-1-yn-1-yl)-1-oxoisoindolin-2-yl)piperidine-2,6-dione (C1)

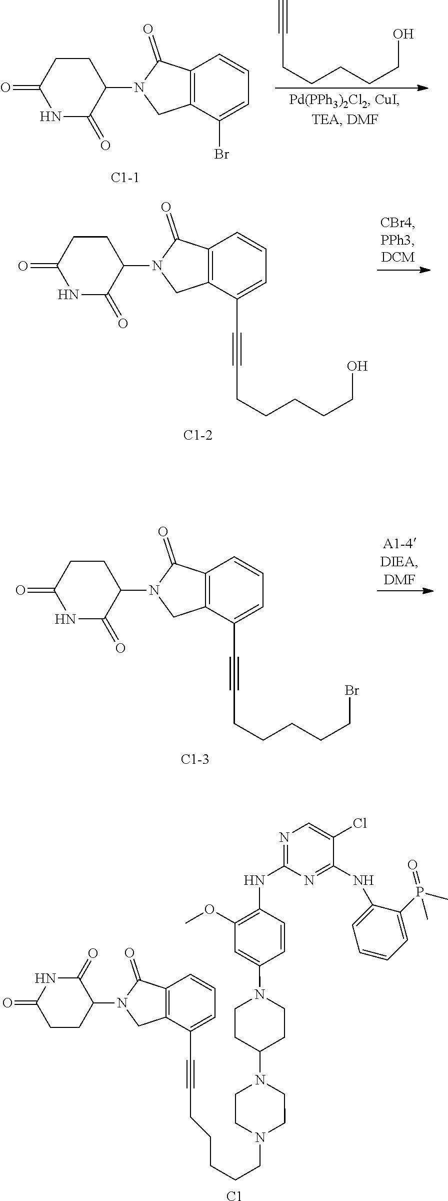

### Step 1

C1-1 (322 mg, 1 mmol), hept-6-yn-1-ol (280 mg, 2.50 mmol), CuI (38 mmol, 0.2 mmol), Pd(PPh₃)₂Cl₂ (280 mg, 0.4 mmol) and TEA (303 mg, 3 mmol) were added to DMF (10 mL). The mixture was reacted with stirring at 70° C. under nitrogen protection overnight. The mixture was cooled to room temperature, and 30 mL of water was added. The mixture was extracted with ethyl acetate (20 mL×3). The organic phases were combined, dried with anhydrous sodium sulfate, and filtered. The filtrate was concentrated, and the crude product was purified by Flash chromatography to afford C₁₋₂, 200 mg, yield: 56.5%. LCMS: [M+H]⁺=355.

### Step 2

C1-2 (40 mg, 0.11 mmol) and CBr₄ (75.5 mg, 0.22 mmol) were dissolved in DCM (15 mL). The mixture was stirred at room temperature under nitrogen protection for 1 h. The mixture was cooled to 0° C. A solution of PPh₃ (59.2 mg, 0.22 mmol) in DCM (2 mL) was added dropwise under nitrogen protection and stirred for 0.5 h. The mixture was heated to 50° C. and further stirred for 3 h. The solvent was removed under reduced pressure, and the crude product was purified by Flash chromatography to afford C1-3, 200 mg, yield: 63.8%. LCMS: [M+H]+=417, 419.

### Step 3

C1-3 (10 mg, 0.024 mmol), A1-4 (15 mg, 0.026 mmol) and DIEA (16 mg, 0.124 mmol) were dissolved in DMF (0.5 mL). The mixture was stirred at 80° C. under nitrogen protection for 7 h. The solvent was removed under reduced pressure, and the crude product was purified by Flash chromatography to afford compound C1, 15 mg, yield: 69.1%. LCMS: [M+H]+=906.

### Preparation of 4-(2-(1-(2-(4-(1-(4-((5-chloro-4-((2-(dimethylphosphoryl)phenyl)amino)pyrimidin-2-yl)amino)-3-methoxyphenyl)piperidin-4-yl)piperazin-1-yl)acetyl)piperidin-4-yl)ethoxy)-2-(2,6-dioxopiperidin-3-yl)isoindoline-1,3-dione (C4)

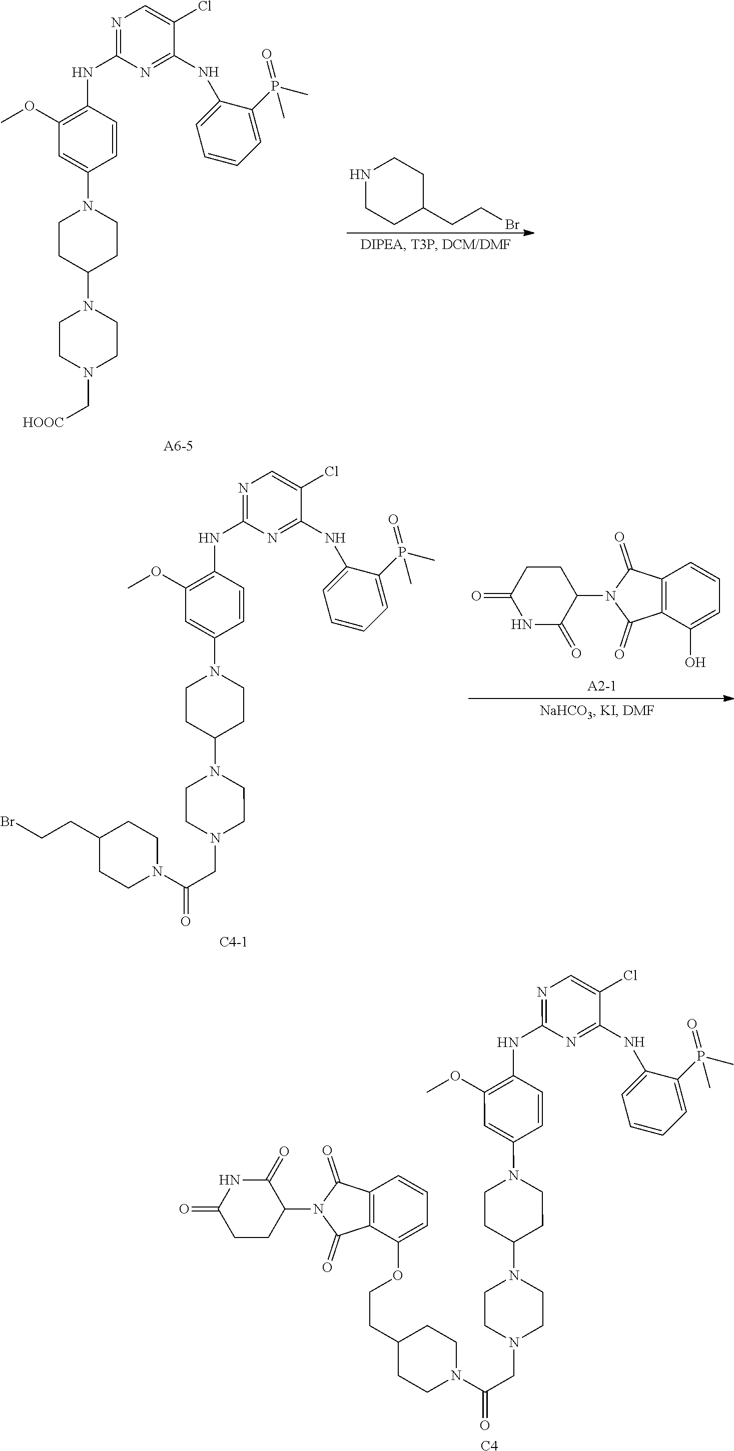

### Step 1

A6-5 (66.3 mg, 0.1 mmol) and 4-(2-bromoethyl)piperidine trifluoroacetate (30.5 mg, 0.1 mmol) were dispersed in 5.0 mL of anhydrous DCM, and T3P (159 mg, 0.25 mmol) and DIPEA (64.5 mg, 0.5 mmol) were added successively. After the addition was completed, the mixture was reacted at room temperature for 2 h. The mixture was diluted with 20 mL of DCM, and then washed successively with saturated NH₄Cl solution (10 mL×1) and saturated NaCl (10 mL×1). The organic layer was dried with anhydrous MgSO₄, and filtered with suction. The filtrate was concentrated under reduced pressure to afford a crude product. The crude product was purified by Prep-TLC to afford light-yellow solid product C4-1, 35 mg. Yield: 43.8%. LCMS: [M+H]⁺=801, 803.

### Step 2

A2-1 (11.3 mg, 0.041 mmol), C4-1 (30.0 mg, 0.038 mmol), KI (3.1 mg, 0.019 mmol) and NaHCO₃ (9.45 mg, 0.113 mmol) were dispersed in 2.0 mL of anhydrous DMF. The mixture was reacted at 70° C. under nitrogen protection for 20 h. LCMS showed that the reaction was completed. The mixture was directly purified by Prep-HPLC to afford light-yellow solid pure product C₄, 13 mg. Yield: 35.1%. LCMS: [M+H]⁺=995.

### Preparation of 2-((2-(2,6-dioxopiperidin-3-yl)-1,3-dioxoisoindolin-4-yl)oxy)acetic acid 3-(4-(1-(4-((5-Chloro-4-((2-(dimethylphosphoryl)phenyl)amino)pyrimidin-2-yl)amino)-3-methoxyphenyl)piperidin-4-yl)piperazin-1-yl)-2,2-dimethyl-3-oxopropyl ester (C₆)

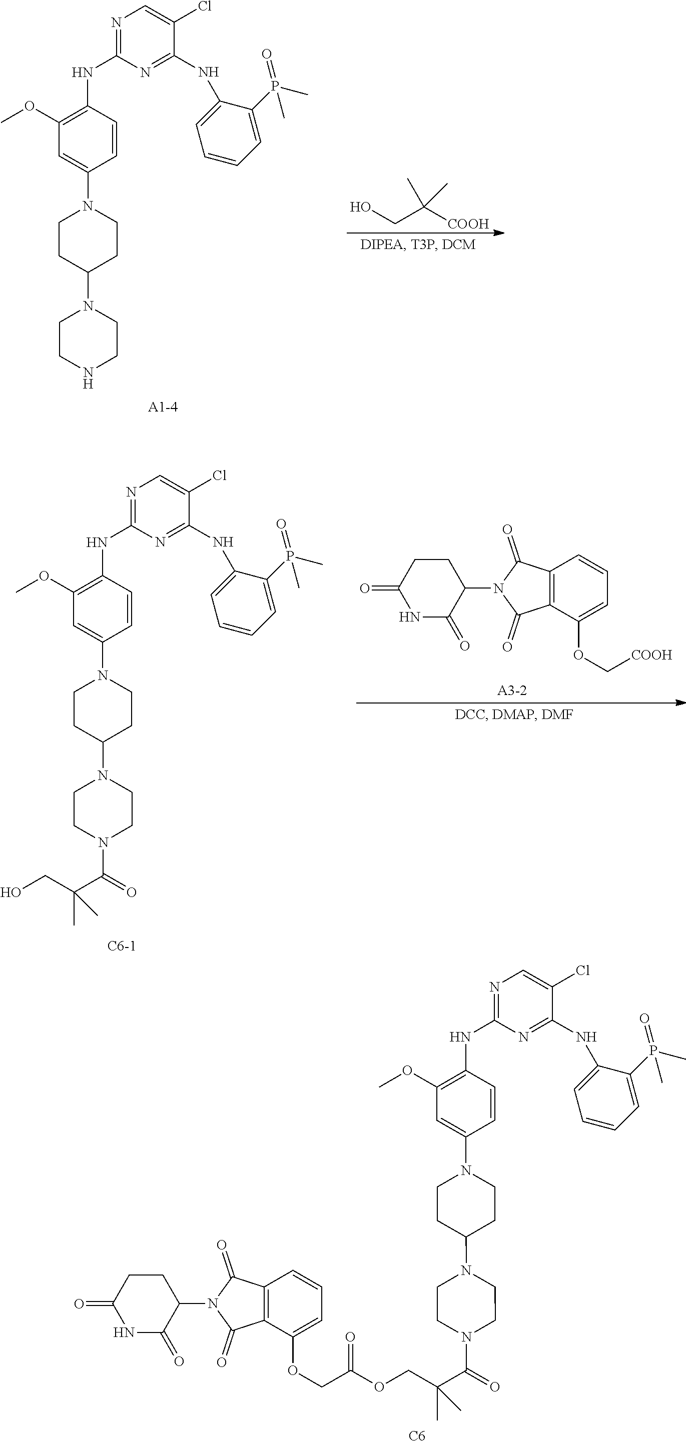

### Step 1

A1-4 (56.9 mg, 0.1 mmol), 3-hydroxy-2,2-dimethylpropionic acid (11.8 mg, 0.1 mmol) and T3P (49.4 mg, 0.13 mmol) were dispersed in 3.0 mL of anhydrous DCM, and DIPEA (51.6 mg, 0.4 mmol) was added with stirring. After the addition was completed, the mixture was reacted at room temperature for 2 h. LCMS showed that the reaction was completed. The mixture was diluted with 30 mL of ethyl acetate, and then washed successively with saturated NH₄Cl solution (20 mL×1) and saturated brine (20 mL×1). The organic layer was dried with anhydrous Na₂SO₄, and filtered with suction. The filtrate was concentrated under reduced pressure to afford a crude product, which was purified by Prep-TLC to afford white solid product C6-1, 65 mg. Yield: 97.0%. LCMS: [M+H]⁺=670.

### Step 2

C6-1 (80.0 mg, 0.1144 mmol), A3-2 (57.0 mg, 0.1716 mmol), DCC (28.3 mg, 0.1373 mmol) and DMAP (14.0 mg, 0.1144 mmol) were dispersed in 6.0 mL of anhydrous DMF. The mixture was stirred at room temperature under nitrogen protection for 8 h. DCC (35.3 mg, 0.1716 mmol) and DMAP (14.0 mg, 0.1144 mmol) were added to the mixture and the reaction was continued at room temperature for 12 h. LCMS showed that the reaction was completed. The mixture was diluted with 50 mL of ethyl acetate, and then washed successively with saturated NH₄Cl solution (20 mL×1), H₂O (20 mL×3) and saturated brine (20 mL×2). The organic layer was dried with anhydrous Na₂SO₄, and filtered with suction. The filtrate was concentrated under reduced pressure to afford a crude product. The crude product was purified by Prep-HPLC to afford 20 mg of light-yellow solid pure product. Yield: 17.8%. LCMS: [M+H]+=984. ¹H-NMR: (400 MHz, DMSO-d₆) δ 11.168 (s, 1H), 11.099 (s, 1H), 8.477 (s, 1H), 8.064 (d, J=7.6 Hz, 2H), 8.064 (t, J=8.0 Hz, 1H), 7.553-7.504 (m, 2H), 7.499-7.321 (m, 3H), 7.111 (t, J=7.6 Hz, 1H), 6.628 (d, J=2.4 Hz, 1H), 6.484 (dd, J=2.4, 8.8 Hz, 1H), 5.123 (s, 2H), 4.164 (s, 2H), 3.760-3.710 (m, 5H), 3.504 (s, 4H), 2.938-2.846 (m, 1H), 2.665-2.455 (m, 8H), 2.373-2.324 (m, 1H), 2.054-1.989 (m, 1H), 1.857-1.746 (m, 8H), 1.565-1.475 (m, 3H), 1.192 (s, 6H).

### Preparation of 4-((9-(4-(1-(4-((4-((2-(dimethylphosphoryl)phenyl)amino)-5-vinylpyrimidin-2-yl)amino)-3-methoxyphenyl)piperidin-4-yl)piperazin-1-yl)nonyl)oxy)-2-(2,6-dioxopiperidin-3-yl)isoindoline-1,3-dione (C25)

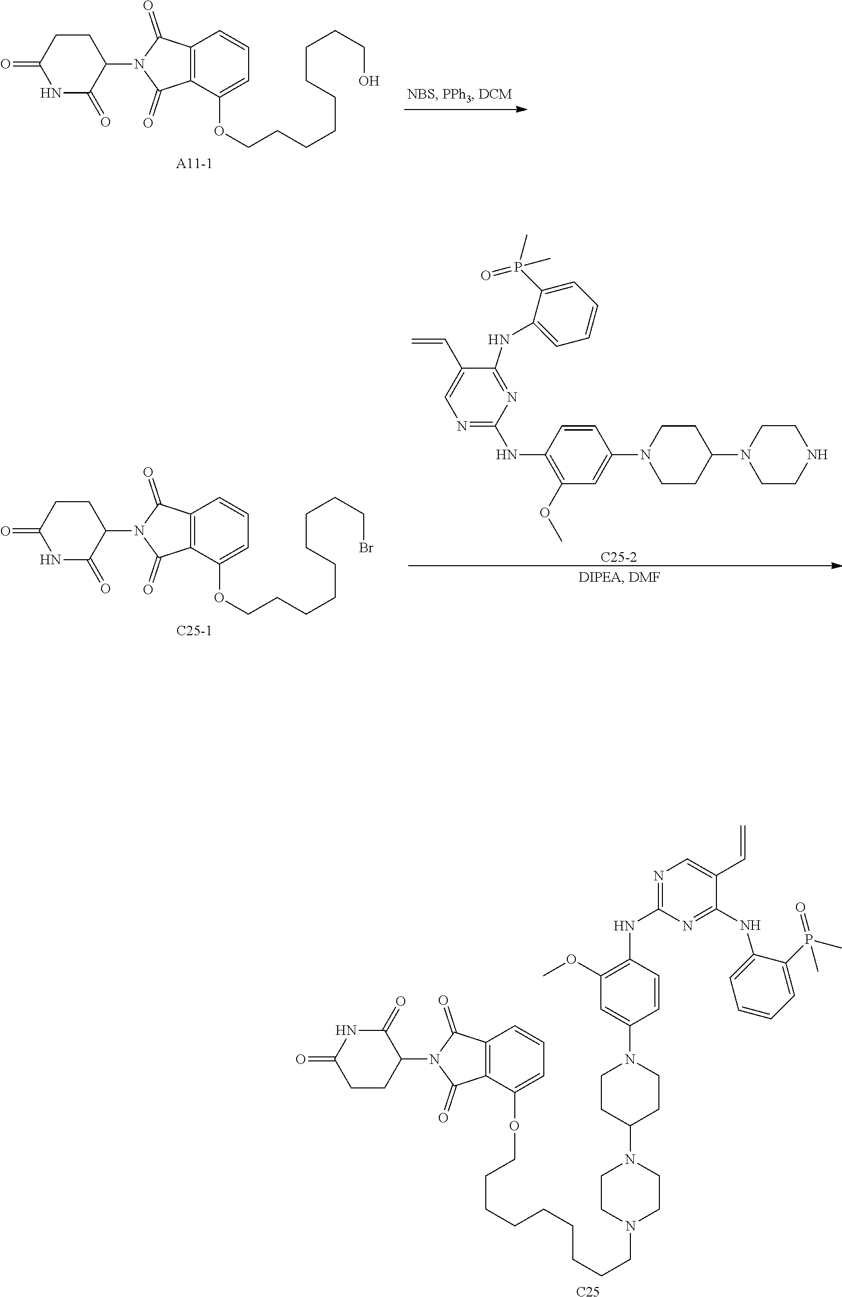

### Step 1

A11-1 (120 mg, 0.288 mmol), PPh₃ (113.4 mg, 0.432 mmol) and NBS (77.0 mg, 0.432 mmol) were dissolved in 8.0 mL of anhydrous DCM. The mixture was reacted at room temperature under N₂ protection for 2 h. TLC showed that there was no raw material remaining. The mixture was diluted with 40 mL of ethyl acetate, and then washed successively with H₂O (20 mL×1) and saturated brine (20 mL×1). The organic layer was dried with anhydrous Na₂SO₄, and filtered with suction. The filtrate was concentrated under reduced pressure to afford a crude product, which was purified by Prep-TLC to afford white solid product C25-1, 100 mg. Yield: 72.6%. LCMS: [M+H]⁺=479, 481.

### Step 2

C25-1 (15 mg, 0.0314 mmol) and C25-2 (17.6 mg, 0.0314 mmol) were dissolved in 2.0 mL of anhydrous DMF. DIPEA (20.3 mg, 0.157 mmol) was added under N₂ protection. After the addition was completed, the mixture was reacted at 80° C. for 8 h. LCMS showed that there was no raw material remaining. The mixture was diluted with 20 mL of ethyl acetate, and then washed successively with saturated NH₄Cl (10 mL×1), H₂O (10 mL×3) and saturated brine (10 mL×1). The organic layer was dried with anhydrous Na₂SO₄, and filtered with suction. The filtrate was concentrated under reduced pressure to afford a crude product. The crude product was purified by Prep-HPLC to afford light-yellow solid product C25, 3.0 mg. Yield: 10.0%. LCMS: [M+1]+=960.

### Preparation of 4-((7-(9-(4-((5-bromo-4-((5-(dimethylphosphoryl)quinoxalin-6-yl)amino)pyrimidin-2-yl)amino)-5-methoxy-2-methylphenyl)-3,9-diazaspiro[5.5]undecan-3-yl)heptyl)oxy)-2-(2,6-dioxopiperidin-3-yl)isoindoline-1,3-dione (C47)

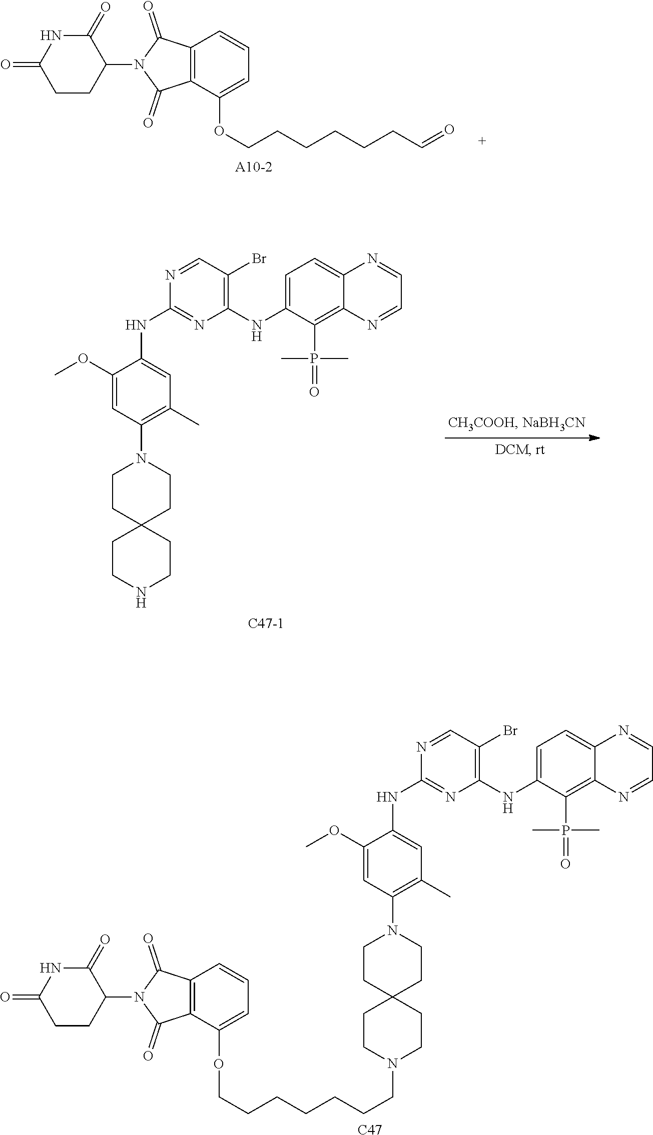 [SMILES: O=CCCCCCCOc1cccc2c1C(=O)N(C1CCC(=O)NC1=O)C2=O ; COc1cc(N2CCC3(CCNCC3)CC2)c(C)cc1Nc1ncc(Br)c(Nc2ccc3nccnc3c2P(C)(C)=O)n1 ; "C47-1" ; COc1cc(N2CCC3(CCN(CCCCCCCOc4cccc5c4C(=O)N(C4CCC(=O)NC4=O)C5=O)CC3)CC2)c(C)cc1Nc1ncc(Br)c(Nc2ccc3nccnc3c2P(C)(C)=O)n1 ; "C47"]

A10-2 (46.3 mg, 0.12 mmol) and C47-1 (66.4 mg, 0.1 mmol) were dissolved in 10.0 mL of anhydrous DCM. CH₃COOH (6.0 mg, 0.1 mmol, dissolved in 0.5 mL of DCM) was added under nitrogen protection. The mixture was stirred at room temperature for 1 h. Then solid NaBH₃CN (15.7 mg, 0.25 mmol) was added and the mixture was further reacted at room temperature for 2 h. LCMS showed that the reaction was completed. The mixture was diluted with 20 mL of DCM, and then washed successively with saturated NH₄Cl (1×15 mL) and saturated brine (1×15 mL). The organic layer was dried with anhydrous Na₂SO₄, and filtered with suction. The filtrate was concentrated under reduced pressure to afford a crude product. The crude product was purified by Prep-HPLC to afford 40 mg of yellow solid pure product. Yield: 38.7%. LCMS: [M+H]⁺=1035, 1037. ¹H-NMR: (400 MHz, DMSO-d₆) δ 12.763 (s, 1H), 11.102 (s, 1H), 9.017 (br s, 1H), 8.886 (d, J=1.6 Hz, 1H), 8.856 (d, J=2.0 Hz, 1H), 8.401 (s, 1H), 8.292 (s, 1H), 7.951 (d, J=9.6 Hz, 1H), 7.841 (t, J=8.0 Hz, 1H), 7.532 (d, J=8.4 Hz, 1H), 7.466 (d, J=7.2 Hz, 1H), 7.380 (s, 1H), 6.868 (s, 1H), 5.102 (dd, J=5.6, 12.8 Hz, 1H), 4.834 (s, 4H), 4.238 (t, J=6.0 Hz, 2H), 3.797 (s, 3H), 3.398-3.369 (m, 2H), 3.097-2.857 (m, 8H), 2.575-2.542 (m, 1H), 2.110 (s, 3H), 2.048-2.012 (m, 7H), 1.804-1.678 (m, 6H), 1.572-1.371 (m, 10H).

### Preparation of 2-((1-(2-(4-(1-(4-((5-chloro-4-((2-(dimethylphosphoryl)phenyl)amino)pyrimidin-2-yl)amino)-3-methoxyphenyl)piperidin-4-yl)piperazin-1-yl)acetyl)piperidin-4-yl)oxy)—N-(2-(2,6-dioxopiperidin-3-yl)-1-oxoisoindolin-4-yl)acetamide (C78)

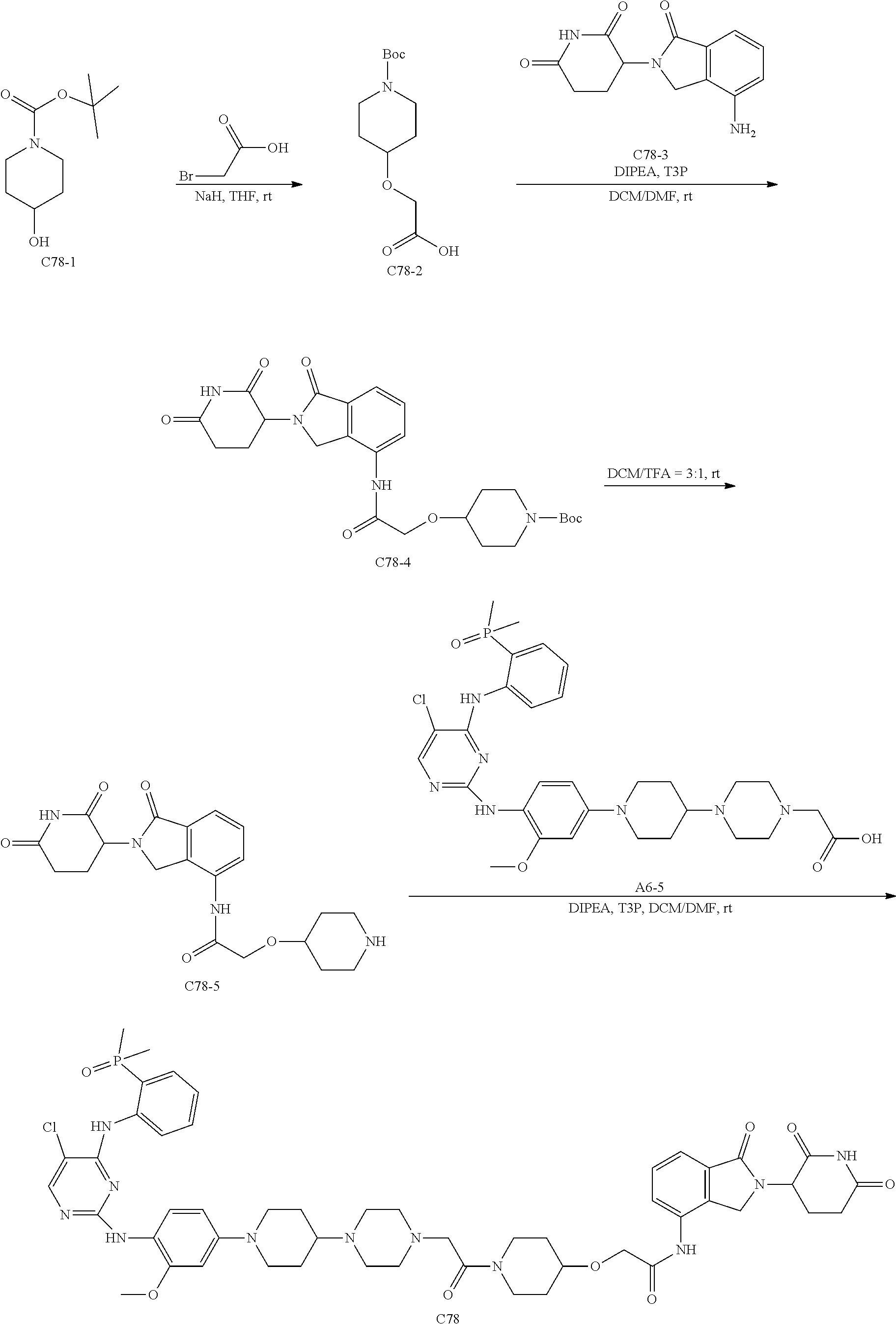

### Step 1

C78-1 (560 mg, 2.78 mmol) was dissolved in 5.0 mL of anhydrous THF, and NaH (222.4 mg, 5.56 mmol) was added at 0° C. The mixture was reacted with stirring at room temperature for 2 h. The mixture was cooled to 0° C., and bromoacetic acid (386 mg, 2.78 mmol, dissolved in 5.0 mL of anhydrous THF) was added dropwise to the mixture. After the dropwise addition, the mixture was warmed to room temperature and reacted for 20 h. The mixture was quenched with water (10 mL) at 0° C. The pH of the mixture was adjusted to 11-12 with 1 M NaOH, and then the mixture was extracted twice with diethyl ether. The pH of remaining aqueous phase was adjusted to 3-4 with 2 N HCl, and then the mixture was extracted with diethyl ether (50 mL×3). The ethyl acetate layers were combined, and washed with saturated brine (50 mL×1). The organic layer was dried with anhydrous Na₂SO₄, and filtered with suction to remove the desiccant. The filtrate was concentrated under reduced pressure to afford crude C78-2 (520 mg, yield: 72.2%), and the crude product was directly used in the next step.

### Step 2

C78-2 (104 mg, 0.4 mmol) and C78-3 (or A12-3) (104 mg, 0.4 mmol) were dispersed in a mixture of 8.0 mL of anhydrous DCM and 1.6 mL of anhydrous DMF, and T3P (508.8 mg, 0.8 mmol) and DIPEA (258 mg, 2.0 mmol) were added successively under nitrogen protection. The mixture was reacted at room temperature for 2 h. The mixture was diluted with 30 mL of ethyl acetate, and then washed successively with saturated NH₄Cl (20 mL×1), H₂O (20 mL×2) and saturated NaCl (20 mL×2). The organic layer was dried with anhydrous MgSO₄, and filtered with suction. The filtrate was concentrated under reduced pressure to afford a crude product, which was purified by Flash chromatography to afford light-yellow oily product C78-4 (120 mg, yield: 60%).

### Step 3

C78-4 (120 mg, 0.24 mmol) was dissolved in 3.0 mL of anhydrous DCM, and 1.0 mL of TFA was added with stirring. The mixture was reacted at room temperature for 2.0 h. LCMS showed that the reaction was completed. The mixture was concentrated under reduced pressure to afford a crude product, which was purified by RP-Flash chromatography to afford white solid product C78-5 (39 mg, yield: 52.1%). LCMS: [M+H]⁺=401.

### Step 4

C78-5 (50 mg, 0.125 mmol) and A6-5 (78.4 mg, 0.125 mmol) were dispersed in a mixture of 5.0 mL of anhydrous DCM and 1.0 mL of anhydrous DMF. T3P (159 mg, 0.25 mmol) and DIPEA (80.6 mg, 0.625 mmol) were added under nitrogen protection. The mixture was stirred at room temperature for 2 h. The mixture was concentrated under reduced pressure to afford a crude product, which was purified by RP-Flash chromatography to afford yellow solid pure product C78 (20 mg, yield: 15.9%). LCMS: [M+H]⁺=1010.

### Preparation of 2-(4-(1-(4-((5-chloro-4-((2-(dimethylphosphoryl)phenyl)amino)pyrimidin-2-yl)amino)-3-methoxyphenyl)piperidin-4-yl)piperazin-1-yl)-N-(1-(2-((2-(2,6-dioxopiperidin-3-yl)-1-oxoisoindolin-4-yl)amino)-2-oxoethyl)piperidin-4-yl)acetamide (C88)

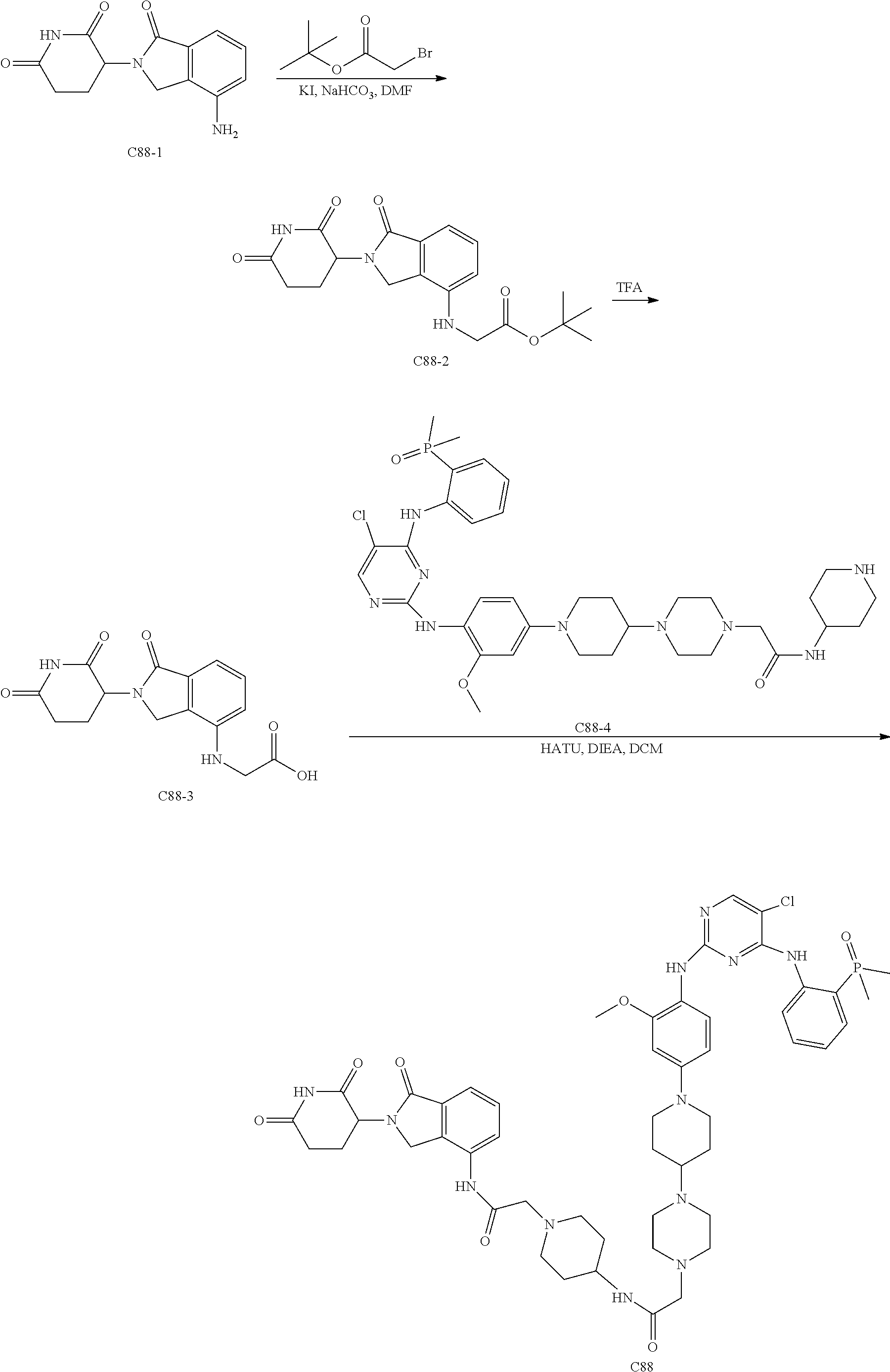

### Step 1

Compound C88-1 (or A12-3) (259.1 mg, 1 mmol), tert-butyl bromoacetate (232.8 mg, 1.2 mmol), potassium iodide (16.6 mg, 0.1 mmol) and sodium bicarbonate (126.0 mg, 1.5 mmol) were added to dry DMF (7 mL). The mixture was stirred at 60° C. under nitrogen protection overnight. The solvent was removed under reduced pressure. The crude product was separated by column chromatography to afford white solid compound C88-2 (150 mg, yield: 40.2%).

### Step 2

Compound C88-2 (44 mg, 0.118 mmol) was dissolved in 1 mL of TFA at 0° C. The mixture was stirred at room temperature for 1 h. The solvent was removed at 40° C. under reduced pressure, and 3 mL of toluene was added. The solvent was evaporated to afford crude C88-3, which was directly used in the next step. LCMS: [M+H]⁺=318.

### Step 3

The above crude C88-3 was dispersed in dichloromethane (2 mL) at room temperature, and HATU (9.2 mg, 0.024 mmol) was added. The mixture was stirred at room temperature under nitrogen protection for 0.5 h. DIEA (15 mg, 0.116 mmol) and compound C88-4 (51 mg, 0.072 mmol) were then added and the mixture was further stirred at room temperature for 4.5 h. After the reaction was completed, the solvent was removed at 40° C. under reduced pressure. The crude product was purified by Flash chromatography to afford off-white solid C88 (5 mg, yield: 19.7%). LCMS: [M+H]+=1009.

### Preparation of 4-((1-(2-(4-(1-(4-((5-chloro-4-((2-(dimethylphosphoryl)phenyl)amino)pyrimidin-2-yl)amino)-3-methoxyphenyl)piperidin-4-yl)piperazin-1-yl)acetyl)azetidin-3-yl)oxy)-2-(2,6-dioxopiperidin-3-yl)isoindoline-1,3-dione (C93)

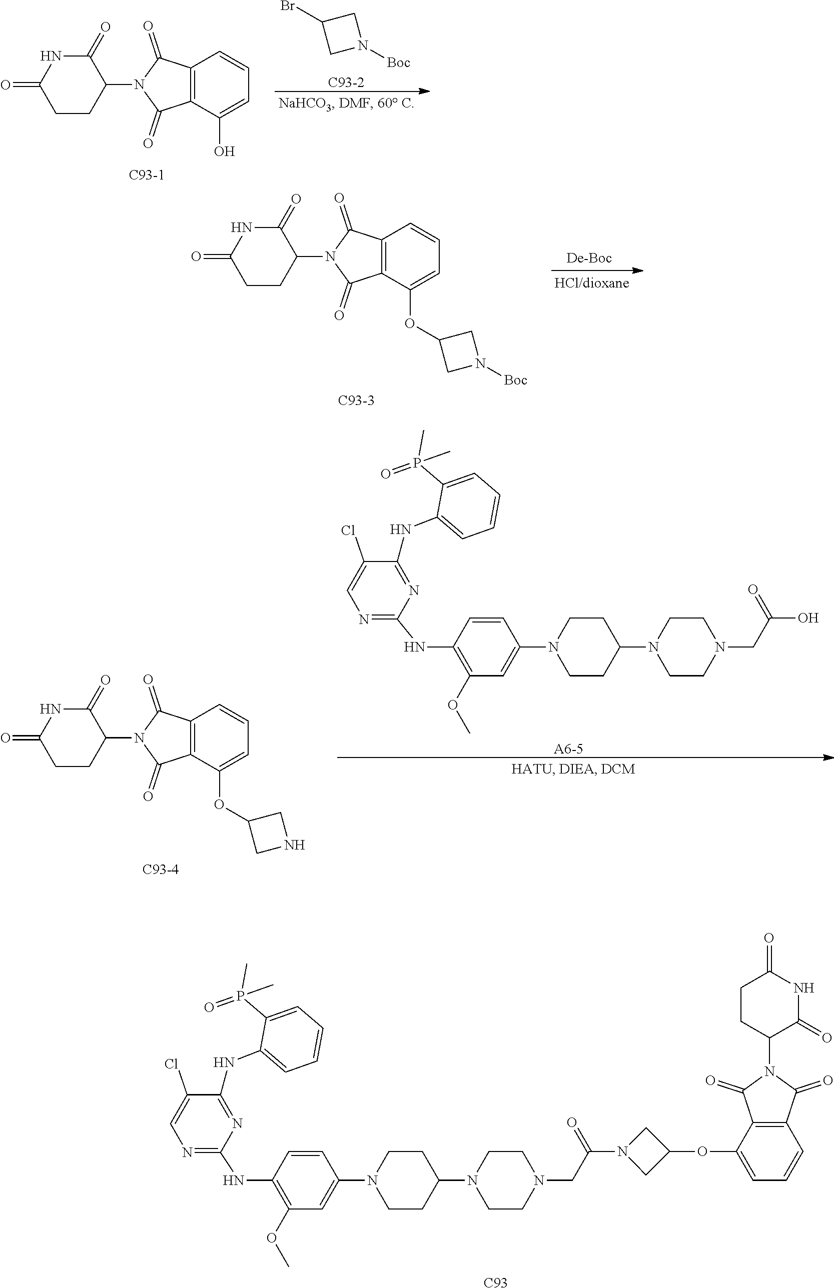

### Step 1

Compound C93-1 (or A2-1) (116 mg, 0.42 mmol), compound C93-2 (100 mg, 0.42 mmol) and sodium bicarbonate (106 mg, 1.26 mmol) were added to a 50 mL single-necked flask, and then DMF (3 mL) was added. The mixture was heated to 60° C. and reacted for 12 h. After the mixture was cooled, water (30 mL) was added. The mixture was extracted with ethyl acetate for three times (20 mL for each time). The organic phases were combined, washed with brine (15 mL×3), dried with anhydrous sodium sulfate, and filtered. The filtrate was concentrated under reduced pressure to afford a crude product. The crude product was separated by column chromatography to afford light white solid C93-3, 110 mg, yield: 61%.

### Step 2

Compound C93-3 (110 mg, 0.256 mmol) and HCl/1,4-dioxane solution (5 mL, 4 mol/L) were added to a 25 mL single-necked flask. The mixture was reacted with stirring at room temperature for 2 h. The reaction process was monitored by TLC. After the reaction was completed, the solvent was concentrated under reduced pressure, and the residue was dissolved with dichloromethane (20 mL). Sodium bicarbonate solution was added to adjust the pH to 8-9. After extraction and separation, the dichloromethane solution was dried with anhydrous sodium sulfate, and filtered. The filtrate was concentrated under reduced pressure to afford light-yellow solid C93-4, 51 mg, yield: 60.7%. LCMS: [M+H]⁺=330.

### Step 3

Compound C93-4 (20 mg, 0.06 mmol), compound A6-5 (38 mg, 0.06 mmol), HATU (27.3 mg, 0.072 mmol) and DIPEA (38.7 mg, 0.03 mmol) were added to a 25 mL single-necked flask, and dichloromethane (2 mL) was added. The reaction was stirred for 3 h. After the reaction was completed, dichloromethane (10 mL) was added, and the mixture was washed with water (5 mL×2). The organic phases were combined, washed with brine (5 mL×2), dried with anhydrous sodium sulfate, and filtered. The filtrate was concentrated under reduced pressure to afford a crude product, which was separated by Prep-HPLC to afford light white solid C93, 5.6 mg, yield: 10%. LCMS: [M+H]⁺=939.

### Preparation of 4-((1-(2-(4-(1-(4-((5-chloro-4-((2-(dimethylphosphoryl)phenyl)amino)pyrimidin-2-yl)amino)-3-methoxyphenyl)piperidin-4-yl)piperazin-1-yl)acetyl)azetidin-3-yl)methoxy)-2-(2,6-dioxopiperidin-3-yl)isoindoline-1,3-dione (C94)

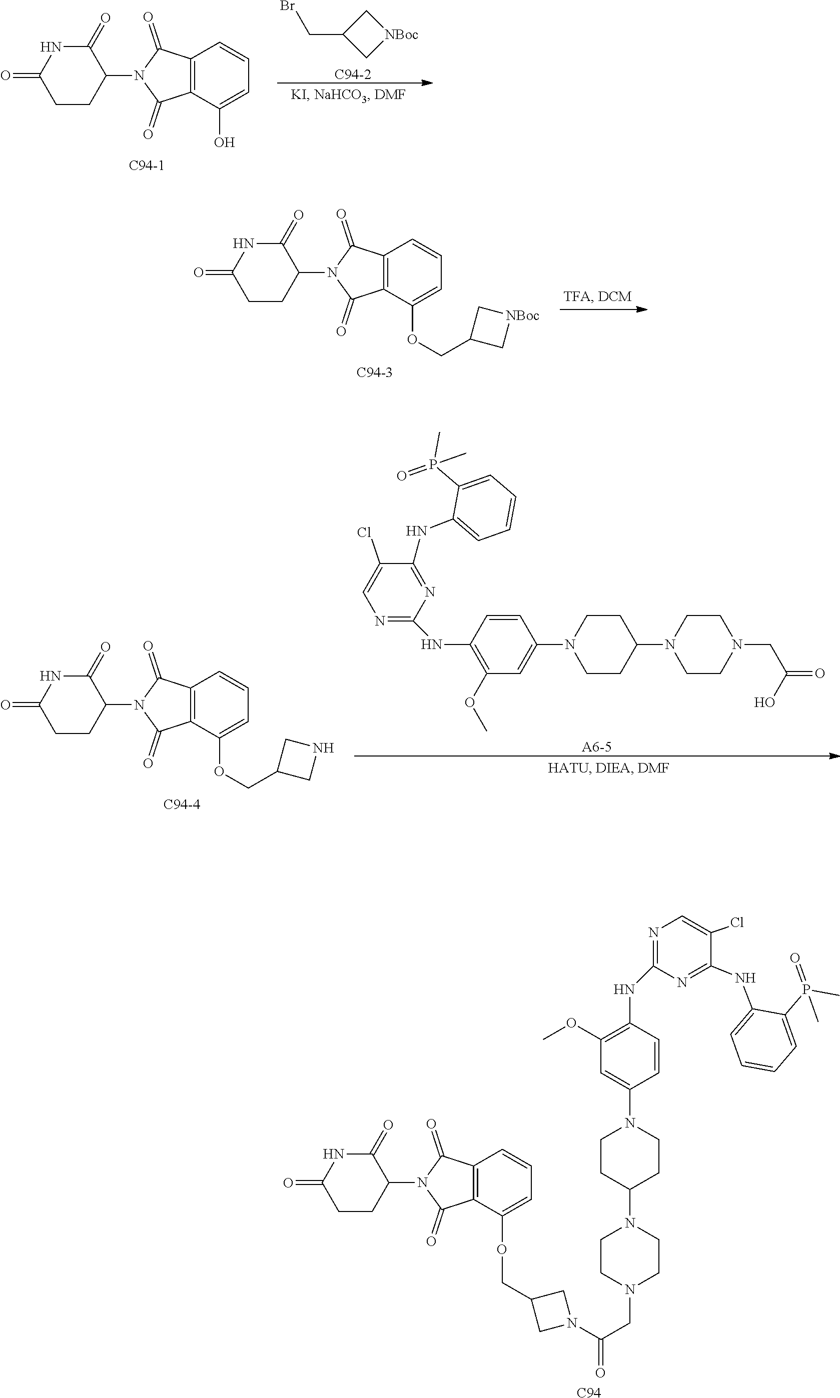

### Step 1

Compound C94-1 (or A2-1) (100 mg, 0.365 mmol), compound C94-2 (109 mg, 0.438 mmol), potassium iodide (6 mg, 0.036 mmol) and sodium bicarbonate (61 mg, 0.726 mmol) were added to dry DMF (3 mL). The mixture was stirred at 60° C. under nitrogen protection for 24 h. The solvent was removed under reduced pressure. The crude product was separated by column chromatography to afford white solid compound C94-3 (50 mg, yield: 31.6%).

### Step 2

Compound C94-3 (50 mg, 0.113 mmol) was dissolved in a solution of trifluoroacetate in dichloromethane (1 mL). The mixture was stirred at room temperature for 2 h. The solvent was removed at 40° C. under reduced pressure to afford crude C94-4, which was directly used in the next step. LCMS: [M+H]⁺=344.

### Step 3

The above crude C94-4 was dispersed in DMF (2 mL) at room temperature, and DIEA (53 mg, 0.411 mmol) and HATU (24 mg, 0.063 mmol) were added. The mixture was stirred at room temperature under nitrogen protection for 1 h. Compound A6-5 (40 mg, 0.064 mmol) was then added and the mixture was further stirred at room temperature for 2.5 h. After the reaction was completed, the solvent was removed at 40° C. under reduced pressure, and the crude product was purified by Flash chromatography to afford off-white solid C94 (20 mg, yield: 36.0%). LCMS: [M+H]+=953. ¹H NMR (400 MHz, DMSO-d₆) δ 11.16 (s, 1H), 11.08 (s, 1H), 8.47 (d, 1H), 8.04 (d, 2H), 7.87-7.81 (m, 1H), 7.57-7.47 (m, 3H), 7.43-7.30 (m, 2H), 7.09 (t, 1H), 6.62 (t, 1H), 6.47 (d, 1H), 5.08 (m, 1H), 4.37 (d, 2H), 4.32 (t, 1H), 4.14 (m, 1H), 3.98 (t, 1H), 3.76 (s, 4H), 3.71 (d, 2H), 3.10-3.04 (m, 1H), 2.97 (s, 2H), 2.95-2.83 (m, 2H), 2.70-2.60 (m, 4H), 2.57 (s, 1H), 2.44 (s, 3H), 2.08-1.94 (m, 2H), 1.83 (d, 2H), 1.78 (s, 3H), 1.74 (s, 3H), 1.51 (d, 2H), 1.25 (m, 3H).

### Preparation of 4-(2-(4-(2-(4-(1-(4-((5-chloro-4-((2-(dimethylphosphoryl)phenyl)amino)pyrimidin-2-yl)amino)-3-methoxyphenyl)piperidin-4-yl)piperazin-1-yl)-2-oxoethyl)piperazin-1-yl)-2-oxoethoxy)-2-(2,6-dioxopiperidin-3-yl)isoindoline-1,3-dione (C99)

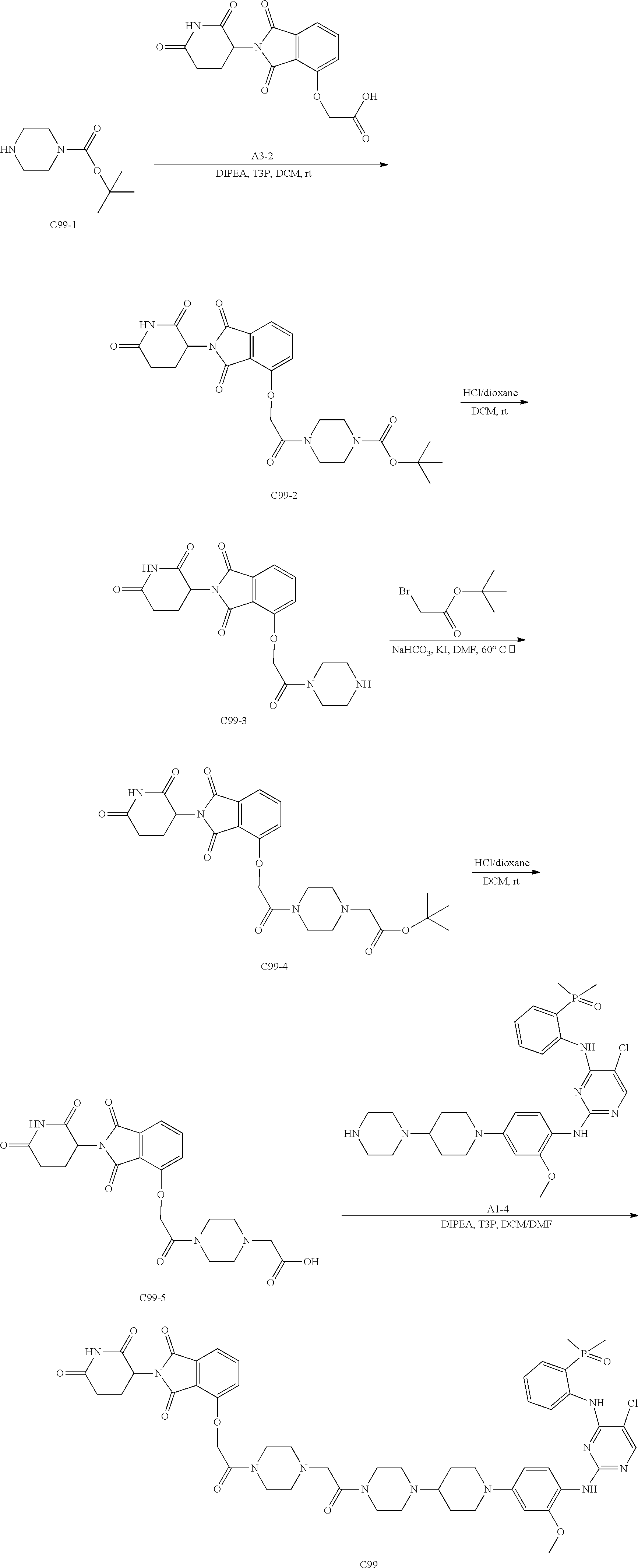

### Step 1

A3-2 (223 mg, 0.5 mmol) and C99-1 (93 mg, 0.5 mmol) were dispersed in 10.0 mL of anhydrous DCM, and T3P (636 mg, 1.0 mmol) and DIPEA (322.5 mg, 2.5 mmol) were added successively under nitrogen protection. The mixture was reacted at room temperature for 2 h. The mixture was diluted with 30 mL of ethyl acetate, and then washed successively with saturated NH₄Cl (20 mL×1) and saturated NaCl (20 mL×2). The organic layer was dried with anhydrous MgSO₄, and filtered with suction. The filtrate was concentrated under reduced pressure to afford a crude product. The crude product was purified by Flash chromatography to afford white solid product C99-2 (230 mg, yield: 92%).

### Step 2

C99-2 (230 mg, 0.46 mmol) was dissolved in 6.0 mL of anhydrous DCM, and 2.0 mL of HCl/dioxane (4.0 M) was added with stirring. The mixture was reacted at room temperature for 2.0 h. LCMS showed that the reaction was completed. The mixture was concentrated under reduced pressure to afford white solid product C99-3 (250 mg, yield: 100%). LCMS: [M+H]+=401.

### Step 3

C99-3 (109 mg, 0.25 mmol), KI (20.8 mg, 0.125 mmol) and NaHCO₃ (84 mg, 1.0 mmol) were dispersed in 5.0 mL of anhydrous DMF. Tert-butyl bromoacetate (48.8 mg, 0.25 mmol) was added under nitrogen protection. The mixture was reacted at 60° C. for 20 h. TLC showed that the reaction was completed. The mixture was diluted with 30 mL of ethyl acetate, and then washed successively with saturated NH₄Cl (10 mL×1), H₂O (10 mL×2) and saturated NaCl (10 mL×2). The organic layer was dried with anhydrous Na₂SO₄, and filtered with suction. The filtrate was concentrated under reduced pressure to afford a crude product. The crude product was purified by Prep-TLC to afford light-yellow solid product C99-4 (95 mg, yield: 74.2%). LCMS: [M+H]⁺=515.

### Step 4

C99-4 (95 mg, 0.185 mmol) was dissolved in 3.0 mL of anhydrous DCM, and 1.5 mL of HCl/dioxane (4.0 M) was added with stirring. The mixture was reacted at room temperature for 2.0 h, and then filtered with suction to afford light-yellow solid product C99-5 (70 mg, yield: 82.6%). LCMS: [M+H]⁺=459.

### Step 5

C99-5 (70 mg, 0.142 mmol) and A1-4 (64.6 mg, 0.114 mmol) were dispersed in 5.0 mL of anhydrous DCM and 1.0 mL of anhydrous DMF. T3P (180.6 mg, 0.284 mmol) and DIPEA (91.6 mg, 0.71 mmol) were added under nitrogen protection. The mixture was stirred at room temperature for 2 h. The mixture was concentrated under reduced pressure to afford a crude product, which was purified by Prep-HPLC to afford yellow solid pure product C99 (25 mg, yield: 17.5%). LCMS: [M+H]+=1010.

### Preparation of 4-(2-(4-(1-(4-((5-chloro-4-((2-(dimethylphosphoryl)phenyl)amino)pyrimidin-2-yl)amino)-3-methoxyphenyl)piperidin-4-yl)piperazin-1-yl)-2-oxoethoxy)-2-(2,6-dioxopiperidin-3-yl)isoindoline-1,3-dione (C101)

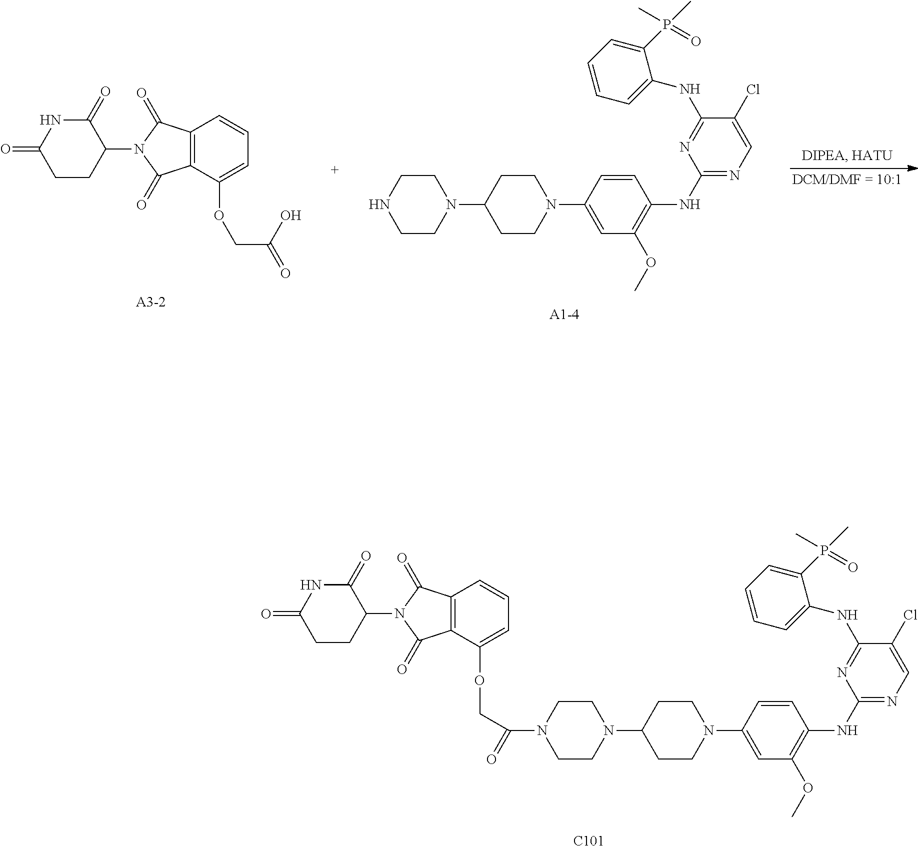 [SMILES: O=C(O)COc1cccc2c1C(=O)N(C1CCC(=O)NC1=O)C2=O ; COc1cc(N2CCC(N3CCNCC3)CC2)ccc1Nc1ncc(Cl)c(Nc2ccccc2P(C)(C)=O)n1 ; COc1cc(N2CCC(N3CCN(C(=O)COc4cccc5c4C(=O)N(C4CCC(=O)NC4=O)C5=O)CC3)CC2)ccc1Nc1ncc(Cl)c(Nc2ccccc2P(C)(C)=O)n1 ; "C101"]

A3-2 (16.6 mg, 0.05 mmol), A1-4 (28.5 mg, 0.05 mmol) and HATU (28.5 mg, 0.075 mmol) were dispersed in a mixture of 2.0 mL of anhydrous DCM and 0.2 mL of anhydrous DMF. DIPEA (32.3 mg, 0.25 mmol) was added dropwise under nitrogen protection. The mixture was stirred at room temperature for 2 h. The mixture was concentrated under reduced pressure to afford a crude product. The crude product was purified by RP-Flash chromatography to afford 30 mg of crude product, which was further purified by Prep-HPLC to afford light-yellow solid pure product C101 (15 mg, yield: 34.1%). LCMS: [M+H]⁺=884.

### Preparation of 2-(((1-(2-(4-(1-(4-((5-chloro-4-((2-(dimethylphosphoryl)phenyl)amino)pyrimidin-2-yl)amino)-3-methoxyphenyl)piperidin-4-yl)piperazin-1-yl)-2-oxoethyl)cyclopropyl)methyl)thio)—N-(2-(2,6-dioxopiperidin-3-yl)-1,3-dioxoisoindolin-4-yl)acetamide (C103)

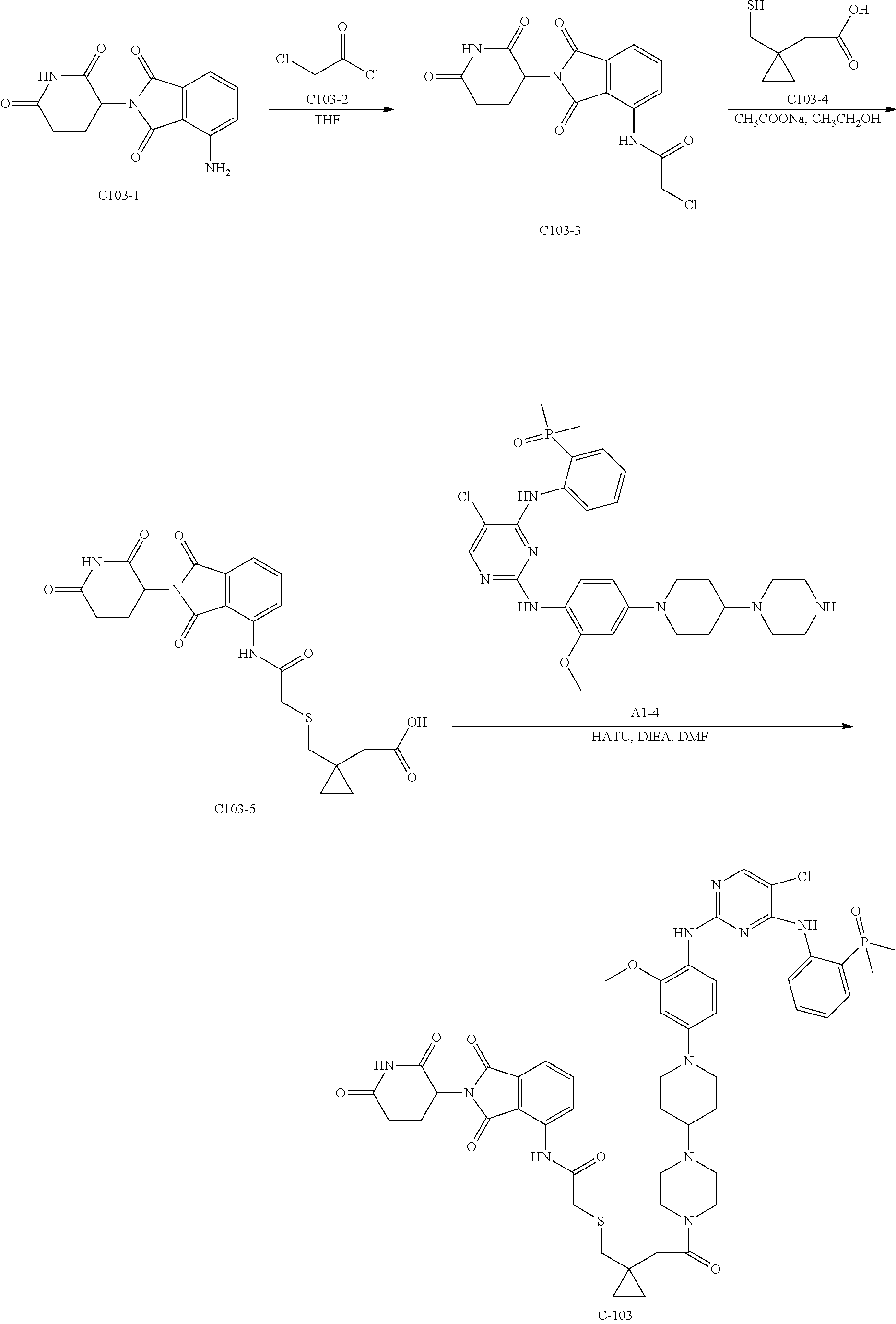

### Step 1

Compound C103-2 (or A8-1) (226 mg, 2.02 mmol) was slowly added to a solution of compound C103-1 (500 mg, 1.83 mmol) in dry THE (10 mL) at 0° C. under nitrogen protection. The mixture was heated and refluxed for 4 h. The mixture was then cooled to room temperature. The solvent was removed under reduced pressure, and the crude product was separated by column chromatography to afford white solid compound C103-3 (300 mg, yield: 46.9%). LCMS: [M+H]+=350.

### Step 2

Compound C103-3 (250 mg, 0.716 mmol), compound C103-4 (105 mg, 0.716 mmol) and sodium acetate (70 mg, 0.854 mmol) were added to anhydrous ethanol (5 mL). The mixture was stirred and refluxed for 3 h. The solvent was removed at 40° C. under reduced pressure, and the crude product was separated by column chromatography to afford compound C103-5 (100 mg, 30.4%). LCMS: [M+H]+=460.

### Step 3

Compound C103-5 (40 mg, 0.087 mmol), compound A1-4 (50 mg, 0.088 mmol), DIEA (34 mg, 0.264 mmol) and HATU (33 mg, 0.087 mmol) were dissolved in 2 mL of DMF at room temperature. The mixture was stirred at room temperature under nitrogen protection for 7 h. LCMS showed that the reaction was completed. The solvent was removed at 40° C. under reduced pressure, and the crude product was purified by Flash chromatography to afford off-white solid C103 (28 mg, yield: 31.8%). LCMS: [M+H]+=1011, ¹H NMR (400 MHz, DMSO-d₆) δ 11.16 (s, 1H), 10.45 (s, 1H), 8.63 (d, 1H), 8.48 (s, 1H), 8.05 (d, 2H), 7.86 (t, 1H), 7.63 (d, 1H), 7.53 (dd, 1H), 7.42-7.31 (m, 2H), 7.09 (t, 1H), 6.62 (d, 1H), 6.47 (d, 1H), 5.75 (s, 1H), 5.17 (dd, 1H), 3.76 (s, 3H), 3.71 (d, 2H), 3.57 (s, 2H), 3.38 (s, 4H), 2.79 (d, 2H), 2.65 (s, 4H), 2.43 (s, 5H), 2.32 (s, 1H), 2.13-2.05 (m, 1H), 2.00 (d, 1H), 1.82 (s, 1H), 1.76 (d, 7H), 1.48 (s, 3H), 0.43 (d, 4H).

### Preparation of 1-(4-(1-(4-((5-chloro-4-((2-(dimethylphosphoryl)phenyl)amino)pyrimidin-2-yl)amino)-3-methoxyphenyl)piperidin-4-yl)piperazine-1-carbonyl)cyclopropane-1-carboxylate 2-(2,6-dioxopiperidin-3-yl)-1,3-dioxoisoindolin-4-yl ester (C104)

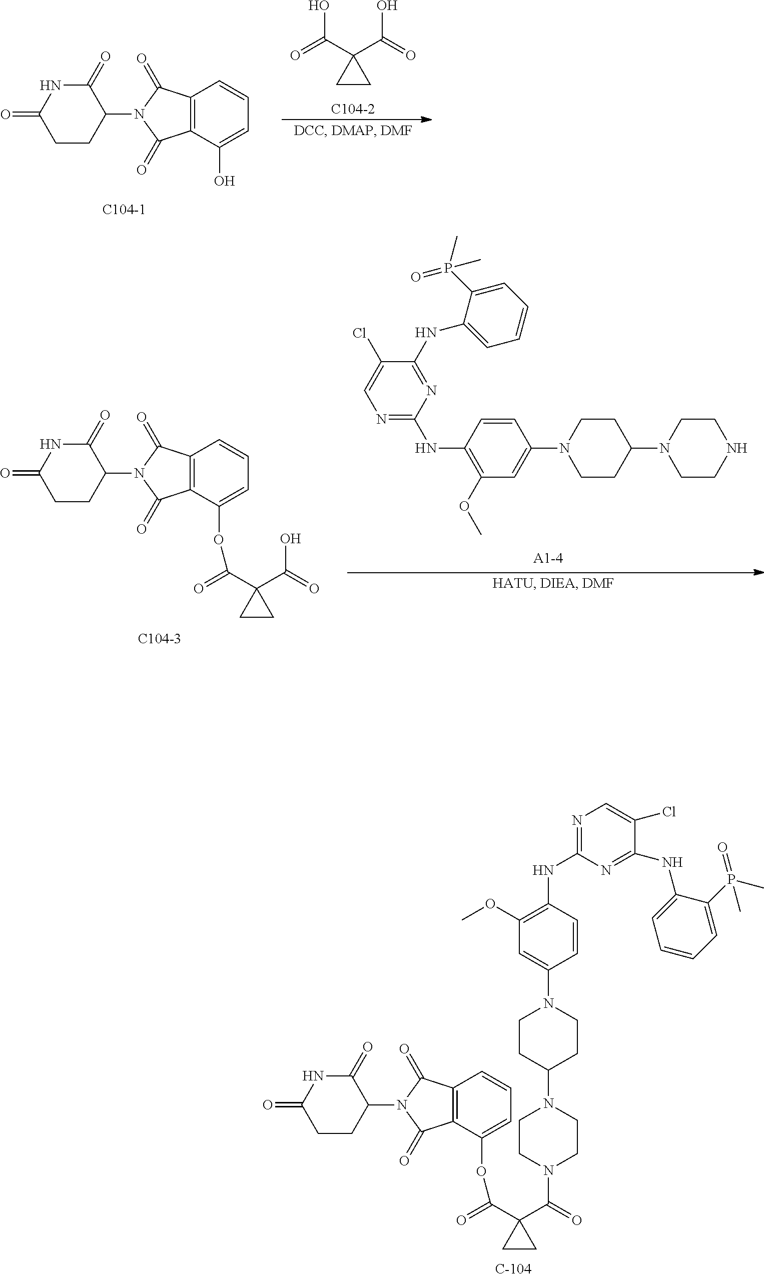

### Step 1

Compound C104-1 (or A2-1) (100 mg, 0.365 mmol), compound C104-2 (119 mg, 0.915 mmol), DCC (90 mg, 0.436 mmol) and DMAP (45 mg, 0.368 mmol) were dissolved in DMF (4 mL). The mixture was stirred at room temperature under nitrogen protection for 5 h. The solvent was removed under reduced pressure, and the crude product was separated by column chromatography to afford white solid compound C104-3 (70 mg, yield: 49.6%).

### Step 2

Compound C104-3 (40 mg, 0.104 mmol), compound A1-4 (56 mg, 0.098 mmol), HATU (40 mg, 0.105 mmol) and DIEA (40 mg, 0.310 mmol) were added to DMF (2 mL). The mixture was stirred at room temperature under nitrogen protection for 3 h. The solvent was removed at 40° C. under reduced pressure, and the crude product was purified by Flash chromatography to afford off-white solid C104 (50 mg, yield: 51.5%). LCMS: [M+H]=938. ¹HNMR (400 MHz, DMSO-d₆) δ 11.14 (d, 2H), 8.48 (s, 1H), 8.06 (d, 2H), 7.96 (t, 1H), 7.87 (d, 1H), 7.70 (d, 1H), 7.56-7.49 (m, 1H), 7.39 (d, 1H), 7.34 (t, 1H), 7.09 (t, 1H), 6.63 (d, 1H), 6.47 (dd, 1H), 5.14 (dd, 1H), 3.76 (s, 3H), 3.72 (d, 4H), 3.51 (s, 2H), 2.89 (dt, 1H), 2.66 (t, 3H), 2.39 (s, 1H), 2.10-1.96 (m, 2H), 1.83 (d, 2H), 1.79-1.73 (m, 9H), 1.55-1.50 (m, 3H), 1.24 (m, 3H).

### Preparation of 4-((1-((4-(1-(4-((5-chloro-4-((2-(dimethylphosphoryl)phenyl)amino)pyrimidin-2-yl)amino)-3-methoxyphenyl)piperidin-4-yl)piperazin-1-yl)methyl)cyclopropyl)methoxy)-2-(2,6-dioxopiperidin-3-yl)isoindoline-1,3-dione (C106)

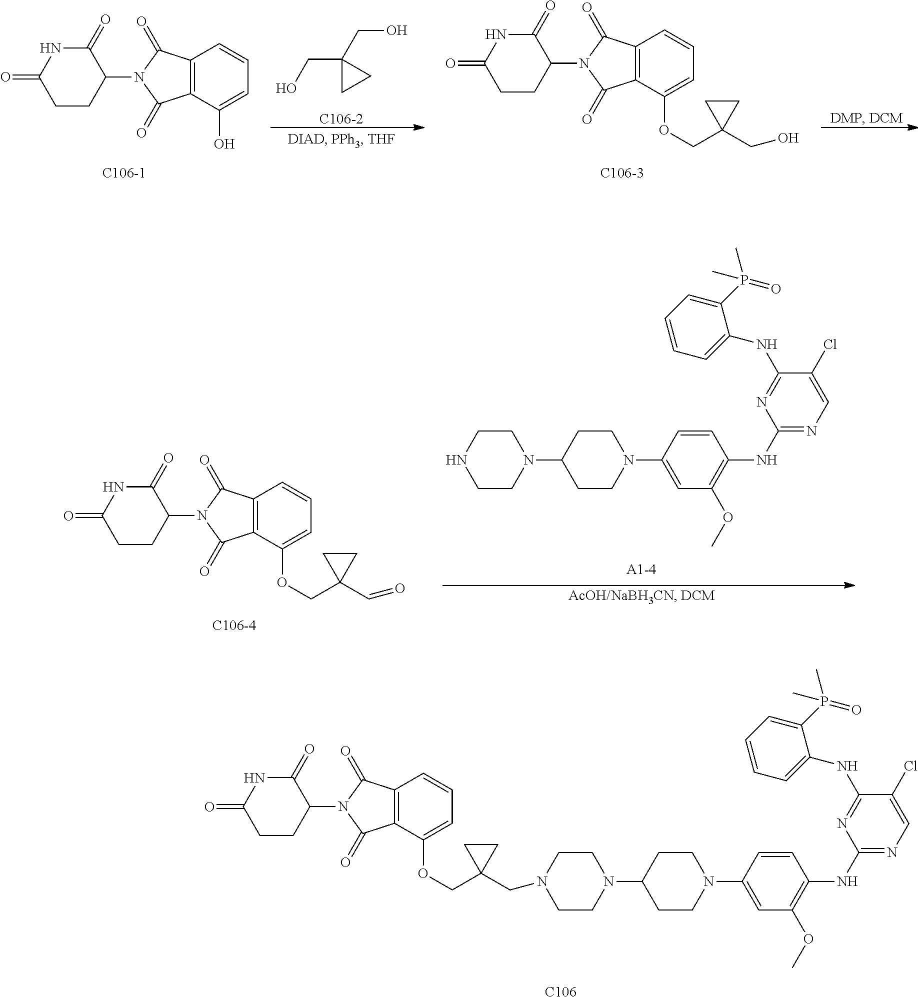

### Step 1

Compound C106-1 (or A2-1) (274 mg, 1 mmol), compound C106-2 (102 mg, 1 mmol), and triphenyl phosphine (393 mg, 1.5 mmol) were added to a 50 mL three-necked flask, and then anhydrous tetrahydrofuran (5 mL) was added. The atmosphere was replaced with nitrogen gas to remove oxygen. The mixture was stirred under nitrogen protection and cooled to 0° C. DIAD (303 mg, 1.5 mmol) was added dropwise. After the addition was completed, the mixture was warmed to room temperature and reacted with stirring for 3 h. The reaction process was monitored by LCMS. The raw materials were basically transformed into the title product. The mixture was concentrated under reduced pressure to afford a crude product, which was separated by column chromatography to afford a title product C106-3, 210 mg, yield: 58.6%. LCMS: [M+H]+=359.

### Step 2

Compound C106-3 (100 mg, 0.28 mmol) was added to a 50 mL single-necked flask and dissolved in dichloromethane (15 mL). Dess-Martin reagent (305 mg, 0.72 mmol) was then added. The mixture was reacted with stirring at room temperature overnight. The reaction process was monitored by LCMS. The raw materials were basically consumed and the title product was produced. 5 mL of saturated sodium bicarbonate solution and 5 mL of saturated sodium thiosulfate solution were successively added to the mixture and stirred for 10 minutes. After the mixture became clear, the layers were separated. The organic phase was dried with anhydrous sodium sulfate, and filtered. The filtrate was concentrated under reduced pressure to afford an off-white solid product, which was separated by Prep-TLC to afford light white solid C106-4, 45 mg, yield: 45%. LCMS: [M+H]+=357.

### Step 3

Compound C106-4 (41 mg, 0.115 mmol) and compound A1-4 (66 mg, 0.115 mmol) were added to a 50 mL single-necked flask and dissolved in dichloromethane (3 mL). CH₃COOH (0.05 mL) was then added. The mixture was reacted with stirring at room temperature for 0.5 h. Then, sodium cyanobohydride (22 mg, 0.345 mmol) was added and reacted overnight. The reaction process was monitored by LCMS. The raw materials were basically consumed and the title product was produced. Saturated NH₄Cl solution (3 mL) was added to the mixture and stirred for 5 minutes. The layers were separated. The organic phase was dried with anhydrous sodium sulfate, and filtered. The filtrate was concentrated under reduced pressure to afford a crude product, which was separated by Prep-TLC to afford white powder C106, 10 mg, yield: 9.6%. LCMS: [M+H]⁺=910.

### Preparation of 2-(4-(1-(4-((5-chloro-4-((2-(dimethylphosphoryl)phenyl)amino)pyrimidin-2-yl)amino)-3-methoxyphenyl)piperidin-4-yl)piperazin-1-yl)acetate (1-(((2-(2,6-dioxopiperidin-3-yl)-1,3-dioxoisoindolin-4-yl)oxy)methyl)cyclopropyl)methyl ester (C107)

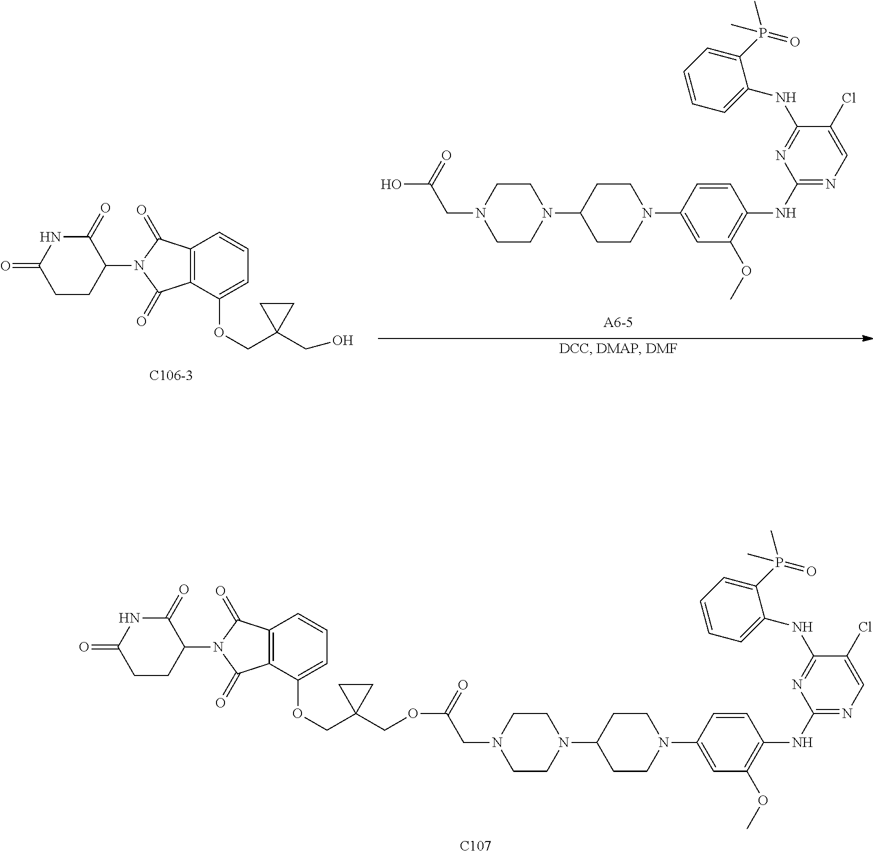

Compound C106-3 (28.5 mg, 0.08 mmol), compound A6-5 (50 mg, 0.08 mmol), DCC (20 mg, 0.096 mmol) and DMAP (10 mg, 0.08 mmol) were added to a 50 mL single-necked flask and dissolved in DMF (3 mL). The mixture was reacted with stirring at room temperature overnight. The reaction process was monitored by LCMS. The reaction was stopped until the raw materials were basically transformed. The mixture was concentrated under reduced pressure to afford a crude product, which was separated by Prep-TLC to afford light-yellow solid product, which was further separated by prep-HPLC to afford white powder C107, 10 mg, yield: 12.98%. LCMS: [M+H]⁺=968.

### Preparation of 4-(((S)-1-(2-(4-(1-(4-((5-chloro-4-((2-(dimethylphosphoryl)phenyl)amino)pyrimidin-2-yl)amino)-3-methoxyphenyl)piperidin-4-yl)piperazin-1-yl)acetyl)pyrrolidin-2-yl)methoxy)-2-(2,6-dioxopiperidin-3-yl)isoindoline-1,3-dione (C109)

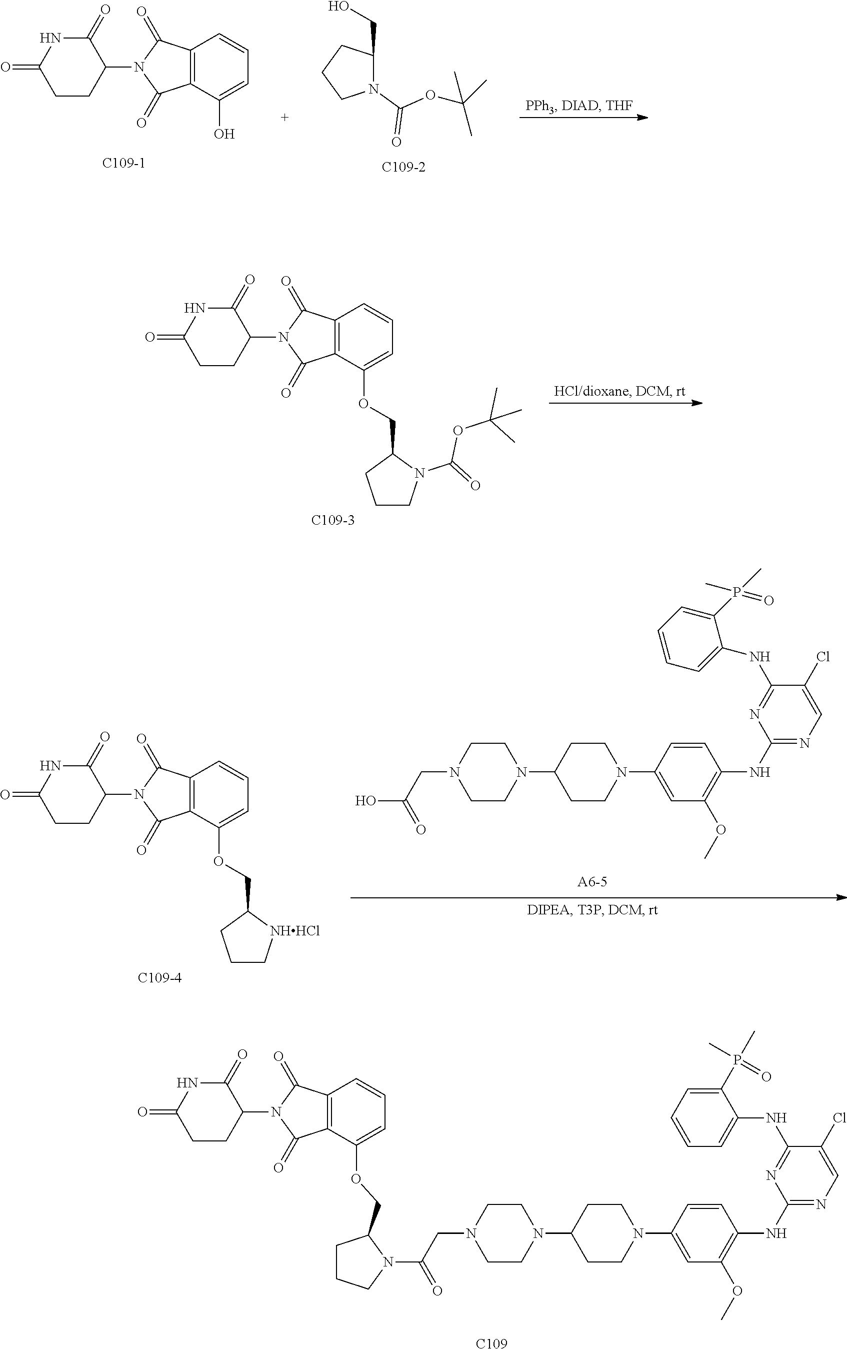

### Step 1

C109-1 (or A2-1) (82.2 mg, 0.3 mmol), C109-2 (66.3 mg, 0.33 mmol) and PPh₃ (94.3 mg, 0.36 mmol) were dispersed in 7.0 mL of anhydrous THF. DIAD (78.8 mg, 0.39 mmol) was added dropwise under nitrogen protection. After the dropwise addition, the mixture was reacted at room temperature for 5.0 h. The mixture was concentrated under reduced pressure to afford a crude product, which was purified by Prep-HPLC to afford white solid product C109-3, 85 mg, yield: 70.0%. LCMS: [M+H]⁺=408.

### Step 2

C109-3 (85 mg, 0.186 mmol) was dissolved in 4.0 mL of anhydrous DCM, and 2.0 mL of 4.0 M HCl/dioxane solution was added. The mixture was sealed and reacted at room temperature for 20 h. LCMS showed that the reaction was completed. The mixture was concentrated under reduced pressure to afford white solid crude C109-4, 100 mg, yield: 100%. The crude product was directly used in the next step. LCMS: [M+H]⁺=358.

### Step 3

C109-4 (42.8 mg, 0.12 mmol) and A6-5 (62.7 mg, 0.1 mmol) were dispersed in 4.0 mL of anhydrous DCM, and T3P (127.2 mg, 0.2 mmol) and DIPEA (64.5 mg, 0.5 mmol) were added successively. The mixture was reacted at room temperature for 2 h. LCMS showed that there was no raw material remaining. The mixture was concentrated under reduced pressure to afford a crude product, which was purified by Prep-HPLC to afford light-yellow solid pure product C109, 18 mg, yield: 18.6%. LCMS: [M+H]+=967. ¹H-NMR: (400 MHz, DMSO-d₆) δ 11.160 (s, 1H), 11.093 (d, J=3.6 Hz, 1H), 8.476 (s, 1H), 8.062 (d, J=13.2 Hz, 2H), 7.861-7.788 (m, 1H), 7.577-7.320 (m, 5H), 7.112 (t, J=7.2 Hz, 1H), 6.622 (d, J=2.8 Hz, 1H), 6.476 (t, J=2.0, 1H), 5.135-5.079 (m, 1H), 4.411-4.238 (m, 3H), 3.761 (s, 3H), 3.707 (d, J=12.0 Hz, 3H), 3.550 (d, J=5.2 Hz, 1H), 3.179-3.122 (m, 1H), 3.005-2.858 (m, 2H), 2.674-2.615 (m, 3H), 2.424-2.202 (m, 9H), 1.997 (s, 4H), 1.813-1.745 (m, 10H), 1.509-1.453 (m, 2H).

### Preparation of (2S)-1-(2-(4-(1-(4-((5-chloro-4-((2-(dimethylphosphoryl)phenyl)amino)pyrimidin-2-yl)amino)-3-methoxyphenyl)piperidin-4-yl)piperazin-1-yl)acetyl)-N-(2-(2,6-dioxopiperidin-3-yl)-1-oxoisoindolin-4-yl)-4,4-difluoropyrrolidine-2-carboxamide (C110)

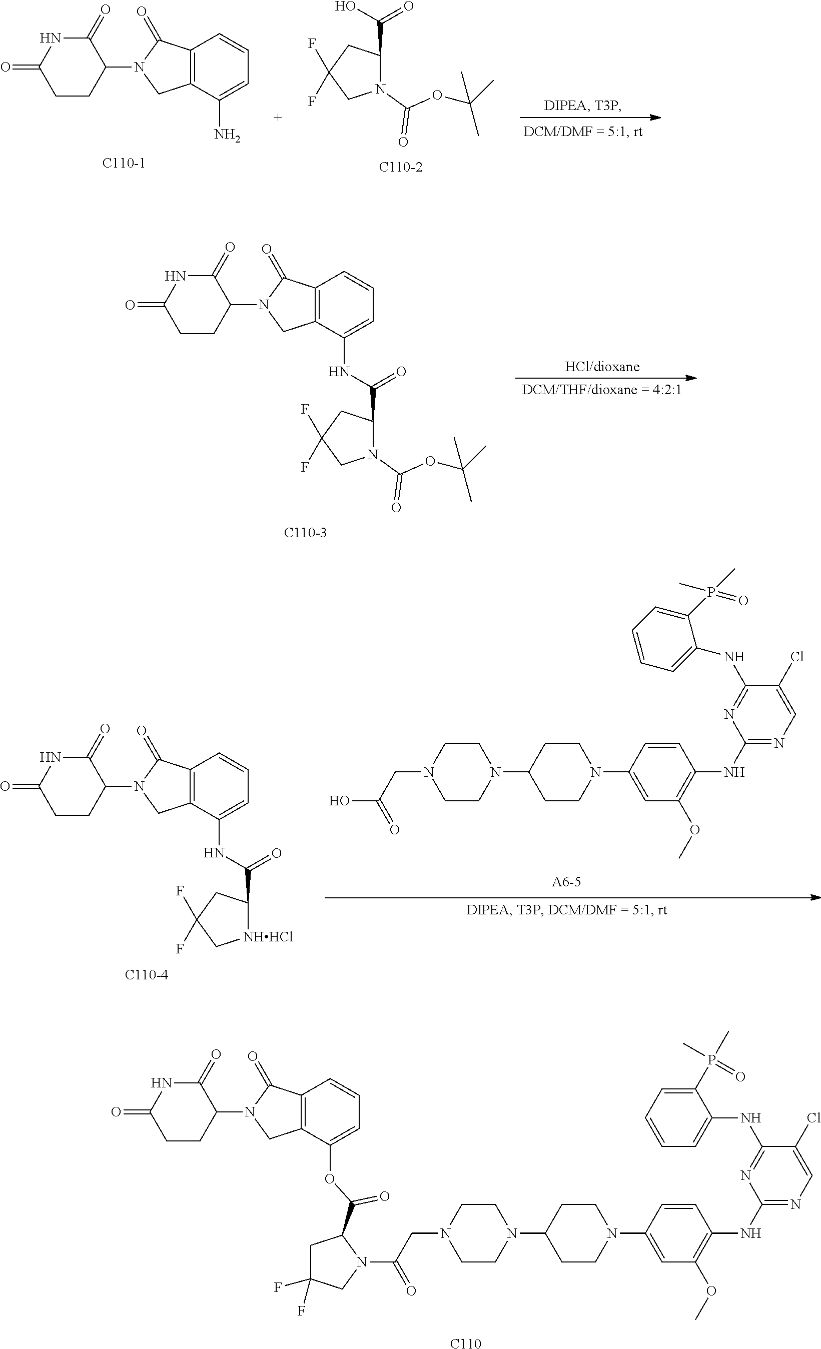

### Step 1

C110-1 (or A12-3) (130 mg, 0.5 mmol) and C110-2 (125.5 mg, 0.5 mmol) were dispersed in a mixture of 6.0 mL of anhydrous DCM and 1.2 mL of anhydrous DMF. T3P (636 mg, 1.0 mmol) and DIPEA (258 mg, 2.0 mmol) were added successively under N₂ protection. The mixture was reacted at room temperature for 2 h. LCMS showed that there was no raw material remaining. The mixture was concentrated under reduced pressure to remove DCM. The residue was diluted with 50 mL of ethyl acetate, and then washed successively with saturated NH₄C₁ solution (20 mL×1), H₂O (20 mL×3) and saturated brine (20 mL×1). The organic layer was dried with anhydrous Na₂SO₄, and filtered with suction. The filtrate was concentrated under reduced pressure to afford a crude product, which was purified by Prep-TLC to afford light-yellow oily product C110-3, 100 mg, yield: 40.7%. ¹H-NMR: (400 MHz, DMSO-d₆) δ 11.021 (s, 1H), 10.080 (s, 1H), 7.781-7.726 (m, 1H), 7.575-7.523 (m, 2H), 5.182-5.136 (m, 1H), 4.574-4.255 (m, 2H), 3.853 (m, 2H), 2.962-2.890 (m, 2H), 2.639-2.585 (m, 1H), 2.341-2.253 (m, 1H), 2.051-2.028 (m, 1H), 1.416 (s, 3H), 1.344 (s, 6H).

### Step 2

C110-3 (100 mg, 0.203 mmol) was dissolved in a mixture of 4.0 mL of anhydrous DCM, 2.0 mL of anhydrous THF and 1.0 mL of anhydrous 1,4-dioxane, and 3.0 mL of 4.0 M HCl/dioxane solution was added. The mixture was sealed and reacted at room temperature for 2 h. LCMS showed that the reaction was completed. The mixture was concentrated under reduced pressure to afford white solid crude C110-4, 130 mg, yield: 100%. The crude product was directly used in the next step. LCMS: [M+H]⁺=393.

### Step 3

A6-5 (51.3 mg, 0.0818 mmol) and C110-4 (35 mg, 0.0818 mmol) were dispersed in a mixture of 5.0 mL of anhydrous DCM and 1.0 mL of anhydrous DMF. T3P (104 mg, 0.1636 mmol) and DIPEA (63.3 mg, 0.4908 mmol) were added successively under N₂ protection. The mixture was reacted at room temperature for 2 h. LCMS showed that there was no raw material remaining. The mixture was concentrated under reduced pressure to afford a crude product, which was purified by Prep-HPLC to afford white solid pure product C110, 25 mg, yield: 30.6%. LCMS: [M+H]+=1002. ¹H-NMR: (400 MHz, DMSO-d₆) δ 11.164 (s, 1H), 11.037 (s, 1H), 10.052 (s, 1H), 8.477 (s, 1H), 8.155-8.043 (m, 2H), 7.776 (t, J=6.8 Hz, 1H), 7.566-7.499 (m, 3H), 7.391-7.318 (m, 2H), 7.112-7.075 (m, 2H), 6.627-6.373 (m, 2H), 5.201-5.130 (m, 1H), 4.505-4.059 (m, 5H), 3.773-3.703 (m, 5H), 3.530 (s, 1H), 3.286-3.275 (m, 2H), 3.095-2.810 (m, 10H), 2.640-2.598 (m, 1H), 2.291-2.180 (m, 4H), 2.008-1.838 (m, 3H), 1.782-1.748 (m, 1H), 1.741 (d, J=12.0 Hz, 6H), 1.538-1.445 (m, 3H).

### Preparation of 2-(4-(1-(4-((5-chloro-4-((2-(dimethylphosphoryl)phenyl)amino)pyrimidin-2-yl)amino)-3-methoxyphenyl)piperidin-4-yl)piperazin-1-yl)-N-((2S)-1-((2-(2,6-dioxopiperidin-3-yl)-1,3-dioxoisoindolin-4-yl)oxy)-3,3-dimethylbutan-2-yl)acetamide (C112)

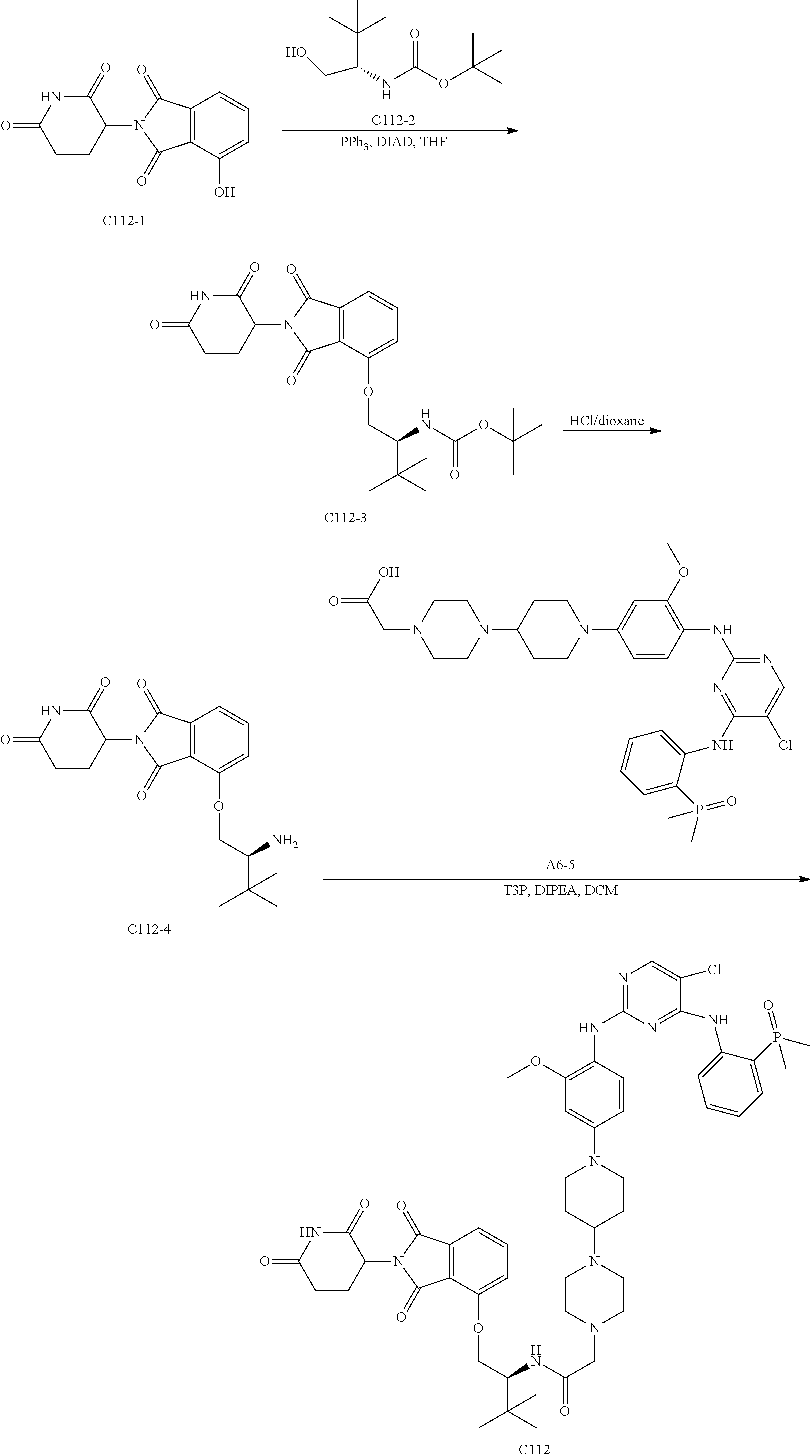

### Step 1

Compound C112-1 (or A2-1) (274 mg, 1 mmol), compound C112-2 (217 mg, 1 mmol), and triphenyl phosphine (393 mg, 1.5 mmol) were added to a 50 mL three-necked flask, and anhydrous tetrahydrofuran (5 mL) was then added. The atmosphere was replaced with nitrogen gas to remove oxygen. The mixture was stirred under nitrogen protection and cooled to 0° C. DIAD (303 mg, 1.5 mmol) was added dropwise. After the addition was completed, the mixture was warmed to room temperature and reacted with stirring for 3 h. The reaction process was monitored by LCMS. After the reaction was completed, the mixture was concentrated under reduced pressure to afford a crude product, which was separated by column chromatography to afford a title product C112-3, 240 mg, yield: 50.7%. LCMS: [M+H]⁺=475.

### Step 2

Compound C112-3 (100 mg, 0.21 mmol) and HCl/1,4-dioxane solution (5 mL, 4 mol/L) were added to a 25 mL single-necked flask. The mixture was reacted with stirring at room temperature for 2 h. The reaction process was monitored by TLC. After the reaction was completed, the mixture was concentrated under reduced pressure to remove HCl and most of the 1,4-dioxane, and dissolved with dichloromethane (20 mL). Sodium bicarbonate solution was added to adjust the pH to 8-9. After separation, the dichloromethane solution was dried with anhydrous sodium sulfate, and filtered. The filtrate was concentrated under reduced pressure to afford light-yellow solid C112-4, 40 mg, yield: 51%. LCMS: [M+H]⁺=374.

### Step 3

Compound C112-4 (40 mg, 0.107 mmol) and A6-5 (67 mg, 0.107 mmol) were added to a 50 mL single-necked flask and dissolved in dichloromethane (3 mL). T₃P (136 mg, 0.214 mmol) was then added. The mixture was reacted with stirring at room temperature for 3 h. The reaction process was monitored by LCMS. After the reaction was completed, dichloromethane (10 mL) was added to the mixture, and the mixture was washed with water (5 mL). The layers were separated. The organic phase was dried with anhydrous sodium sulfate, and filtered. The filtrate was concentrated under reduced pressure to afford a crude product, which was purified by prep-TLC to afford light-yellow solid, which was further separated by prep-HPLC to afford light white powder C112, 34 mg. yield 32.3%. LCMS: [M+H]⁺=983.

### Preparation of 3-(4-(6-(4-(1-(4-((5-chloro-4-((2-(dimethylphosphoryl)phenyl)amino)pyrimidin-2-yl)amino)-3-methoxyphenyl)piperidin-4-yl)piperazin-1-yl)hex-1-yn-1-yl)-1-oxoisoindolin-2-yl)piperidine-2,6-dione (C116)

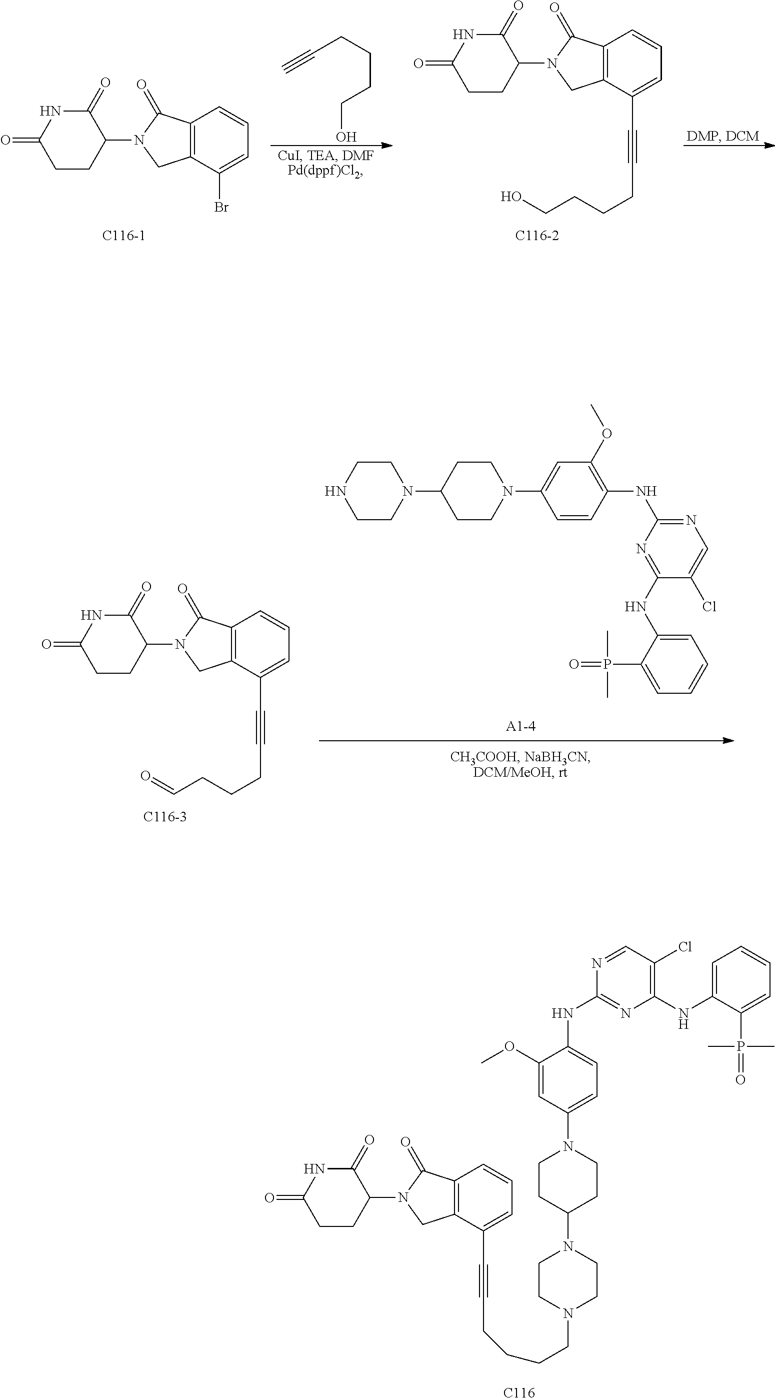

### Step 1

C116-1 (or C1-1) (500 mg, 1.553 mmol), CuI (59 mg, 0.3106 mmol) and Pd(dppf)Cl₂ (454 mg, 0.6212 mmol) were dispersed in 25 mL of anhydrous DMF. 5-alkynyl-1-hexanol (380 mg, 3.883 mmol) and TEA (470 mg, 4.659 mmol) were added successively under N2 protection. The mixture was heated to 70° C. and reacted for 20 h. LCMS showed that the reaction was completed. The mixture was cooled to room temperature, diluted with 150 mL of ethyl acetate, and then washed successively with saturated NH₄Cl (2×50 mL), H₂O (2×50 mL) and saturated brine (2×50 mL). The organic layer was dried with anhydrous sodium sulfate, and filtered with suction. The filtrate was concentrated under reduced pressure to afford a crude product, which was purified by RP-Flash chromatography to afford light-yellow solid product C116-2 (255 mg, 48.3%). LCMS: [M+H]⁺=341.

### Step 2

C116-2 (90 mg, 0.265 mmol) was dispersed in 18.0 mL of anhydrous DCM, and the mixture was heated to 40° C. to make the mixture clear. Dess-Martin reagent (168.5 mg, 0.3975 mmol) was added under N₂ protection. The mixture was heated to 50° C. and refluxed for 2.0 h. TLC showed that the reaction was completed. The mixture was cooled to room temperature, and then 10 mL of saturated NaHCO₃ solution and 10 mL of saturated Na₂S₂O₃ solution were added. The mixture was stirred vigorously at room temperature for 5 min. The organic layer was separated, dried with anhydrous sodium sulfate, and filtered with suction. The filtrate was concentrated under reduced pressure to afford a crude product. The crude product was purified by Prep-TLC to afford light-yellow solid product C116-3 (35 mg, 39.1%). LCMS: [M+H]+=339.

### Step 3

C116-3 (30 mg, 0.0888 mmol) and A1-4 (45.5 mg, 0.0799 mmol) were dissolved in a mixture of 4.0 mL of anhydrous DCM and 0.4 mL of anhydrous methanol. CH₃COOH (5.3 mg, 0.0888 mmol, dissolved in 0.5 mL of DCM) was added under nitrogen protection. The mixture was stirred at room temperature for 0.5 h. Then solid NaBH₃CN (8.36 mg, 0.1332 mmol) was added and the mixture was further reacted at room temperature for 2 h. LCMS and TLC showed that the reaction was completed. The mixture was purified by Prep-HPLC to afford white solid product C116, 25 mg, yield: 31.6%. LCMS: [M+H]+=892. ¹H-NMR: (400 MHz, DMSO-d₆) δ 12.683 (s, 1H), 11.119 (s, 1H), 8.869 (dd, J=2.0 Hz, 9.2 Hz, 3H), 8.281 (s, 1H), 8.263 (s, 1H), 7.721 (d, J=6.8 Hz, 1H), 7.646 (d, J=0.8 Hz, 1H), 7.643 (d, J=6.8 Hz, 1H), 7.539 (d, J=7.6 Hz, 1H), 7.321 (s, 1H), 6.809 (s, 1H), 5.188 (dd, J=5.2 Hz, 13.6 Hz, 1H), 4.477 (d, J=17.6 Hz, 1H), 4.325 (d, J=17.6 Hz, 1H), 3.784 (s, 3H), 2.513-2.495 (m, 10H), 2.494-2.103 (m, 6H), 2.075 (s, 3H), 2.040 (s, 3H), 2.004 (s, 3H), 1.557-1.429 (m, 18H).

### Preparation of 3-(4-(5-(4-(1-(4-((5-chloro-4-((2-(dimethylphosphoryl)phenyl)amino)pyrimidin-2-yl)amino)-3-methoxyphenyl)piperidin-4-yl)piperazin-1-yl)pent-1-yn-1-yl)-1-oxoisoindolin-2-yl)piperidine-2,6-dione (C126)

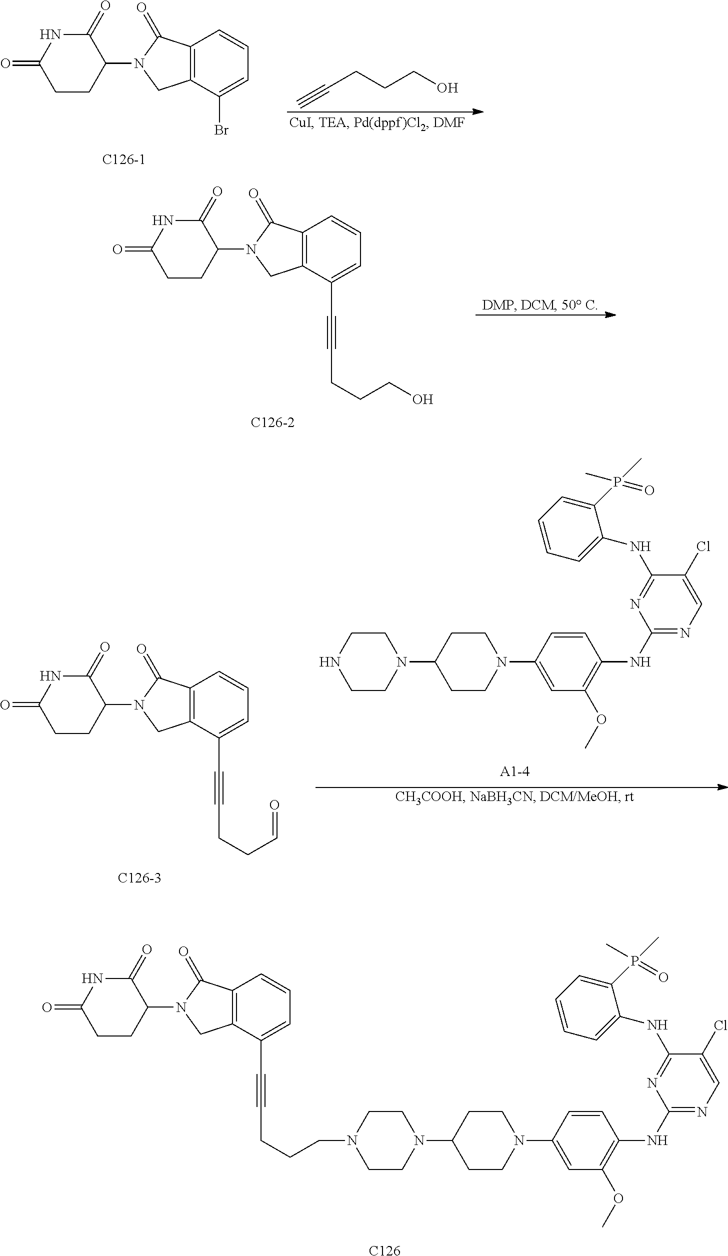

### Step 1

C126-1 (or C1-1) (322 mg, 1.0 mmol), CuI (19 mg, 0.1 mmol) and Pd(dppf)Cl₂ (73.1 mg, 0.1 mmol) were dispersed in 10.0 mL of anhydrous DMF. 4-alkynyl-1-pentanol (210 mg, 2.5 mmol) and TEA (303 mg, 3.0 mmol) were added successively under N₂ protection. The mixture was heated to 70° C. and reacted for 20 h. LCMS showed that the reaction was completed. The mixture was cooled to room temperature and purified by RP-Flash chromatography to afford white solid crude C126-2 (410 mg, yield: 94.1%). LCMS: [M+H]⁺=327.

### Step 2

C126-2 (400 mg, 0.920 mmol) was dissolved in a mixture of 150 mL of anhydrous DCM and 10 mL of anhydrous THF, and Dess-Martin reagent (1.04 g, 2.454 mmol) was added under N₂ protection. The mixture was heated to 50° C. and refluxed for 2.0 h. TLC showed that the reaction was completed. The mixture was cooled to room temperature, and then 20 mL of saturated NaHCO₃ solution and 20 mL of saturated Na₂S₂O₃ solution were added. The mixture was stirred vigorously at room temperature for 5 min. The organic layer was separated, dried with anhydrous sodium sulfate, and filtered with suction. The filtrate was concentrated under reduced pressure to afford a crude product. The crude product was purified by Flash chromatography to afford light-yellow solid product C126-3 (280 mg, 93.3%). LCMS: [M+H]+=325.

### Step 3

C126-3 (48.6 mg, 0.15 mmol) and A1-4 (76.8 mg, 0.135 mmol) were dissolved in a mixture of 5.0 mL of anhydrous DCM and 0.5 mL of anhydrous MeOH. CH₃COOH (13.5 mg, 0.225 mmol) was added under nitrogen protection. The mixture was stirred at room temperature for 0.5 h. Then solid NaBH₃CN (14.1 mg, 0.225 mmol) was added and the mixture was further reacted at room temperature for 2 h. LCMS and TLC showed that the reaction was completed. The mixture was purified by Prep-HPLC to afford white solid pure product C126 (35 mg, yield: 26.7%). LCMS: [M+H]⁺=878.

### Preparation of 4-(7-(4-(1-(4-((5-chloro-4-((2-(dimethylphosphoryl)phenyl)amino)pyrimidin-2-yl)amino)-3-methoxyphenyl)piperidin-4-yl)piperazin-1-yl)hept-1-yn-1-yl)-2-(2,6-dioxopiperidin-3-yl)isoindoline-1,3-dione (C148)

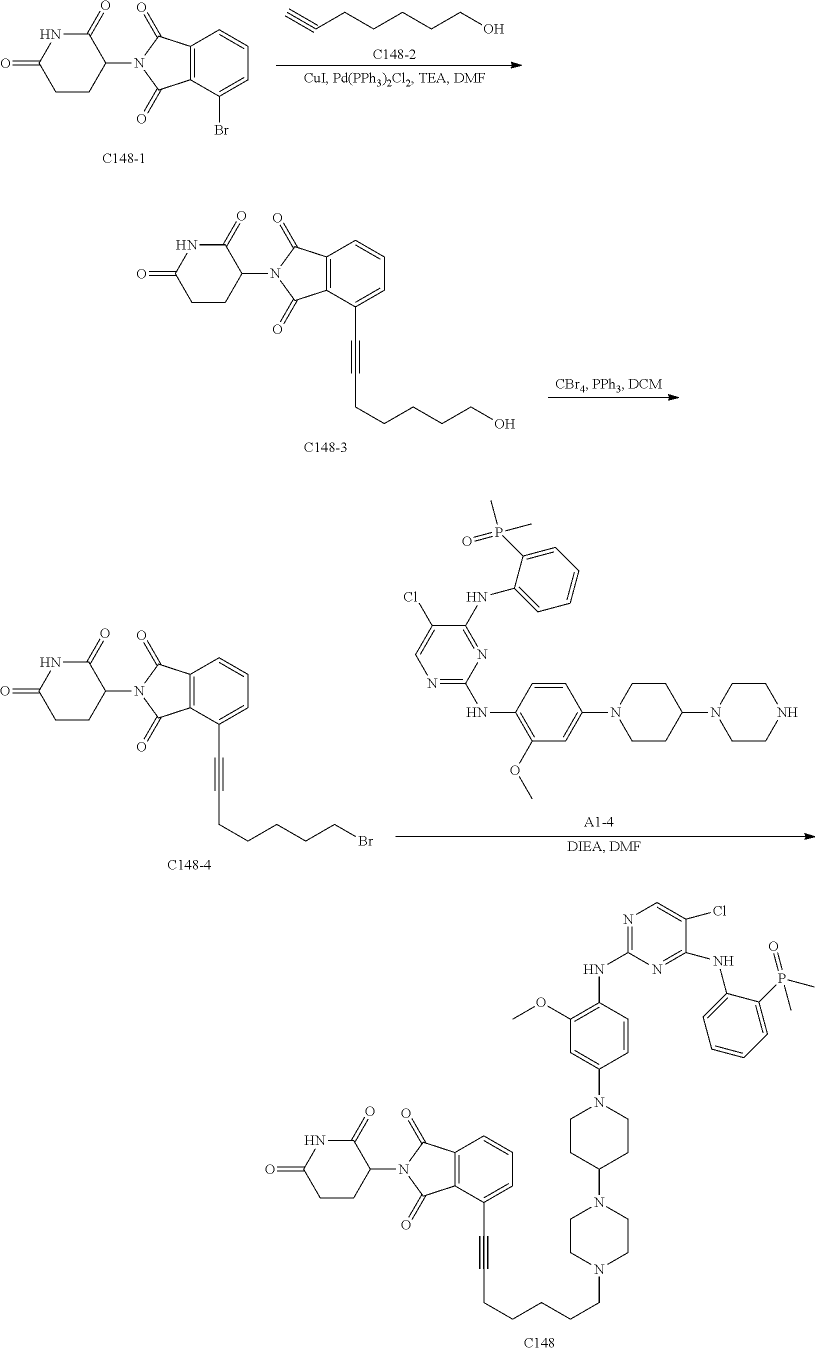

### Step 1

Compound C148-1 (or A1-1) (330 mg, 0.98 mmol), compound C148-2 (220 mg, 1.96 mmol), cuprous iodide (38 mg, 0.20 mmol), Pd(PPh₃)₂Cl₂ (274 mg, 0.39 mmol) and triethylamine (297 mg, 2.94 mmol) were dissolved in dry DMF (10 mL). The mixture was stirred at 70° C. under nitrogen protection overnight. The mixture was cooled to room temperature. The solvent was removed under reduced pressure, and the crude product was separated by column chromatography to afford compound C148-3 (90 mg, yield: 24.9%). LCMS: [M+H]+=369.

### Step 2

Compound C148-3 (60 mg, 0.163 mmol) and CBr₄ (108 mg, 0.326 mmol) were dissolved in dichloromethane (20 mL). The mixture was stirred at room temperature for 2 h. The mixture was cooled to 0° C. A solution of triphenyl phosphine (86 mg, 0.326 mmol) in dichloromethane (2 mL) was added dropwise. The mixture was naturally warmed to room temperature, and reacted with stirring at 55° C. for 2 h. The solvent was removed under reduced pressure, and the crude product was separated by column chromatography to afford compound C148-4 (40 mg, 57.1%). LCMS: [M+H]⁺=431, 433.

### Step 3

Compound C148-4 (40 mg, 0.093 mmol), compound A1-4 (58 mg, 0.102 mmol), and DIEA (60 mg, 0.465 mmol) were dissolved in 2 mL of DMF. The mixture was stirred at 80° C. under nitrogen protection for 5 h. LCMS showed that the reaction was completed. The solvent was removed at 40° C. under reduced pressure. The crude product was purified by Flash chromatography to afford C148 (22 mg, yield: 25.9%). LCMS: [M+H]+=920. ¹H NMR (400 MHz, DMSO-d₆) δ 11.13 (d, 2H), 8.47 (s, 1H), 8.04 (d, 2H), 7.87 (dd, 3H), 7.52 (dd, 1H), 7.36 (dd, 2H), 7.09 (t, 1H), 6.62 (d, 1H), 6.46 (dd, 1H), 5.75 (s, 1H), 5.14 (dd, 1H), 3.76 (s, 3H), 3.71 (d, 2H), 3.26 (s, 1H), 2.92-2.83 (m, 1H), 2.69-2.60 (m, 4H), 2.55 (d, 4H), 2.34 (d, 5H), 2.03 (m, 2H), 1.84 (d, 2H), 1.76 (d, 6H), 1.60 (m, 3H), 1.47 (s, 6H).

### Preparation of 4-(3-((7-(4-(1-(4-((5-chloro-4-((2-(dimethylphosphoryl)phenyl)amino)pyrimidin-2-yl)amino)-3-methoxyphenyl)piperidin-4-yl)piperazin-1-yl)heptyl)oxy)azetidin-1-yl)-2-(2,6-dioxopiperidin-3-yl)isoindoline-1,3-dione (C150)

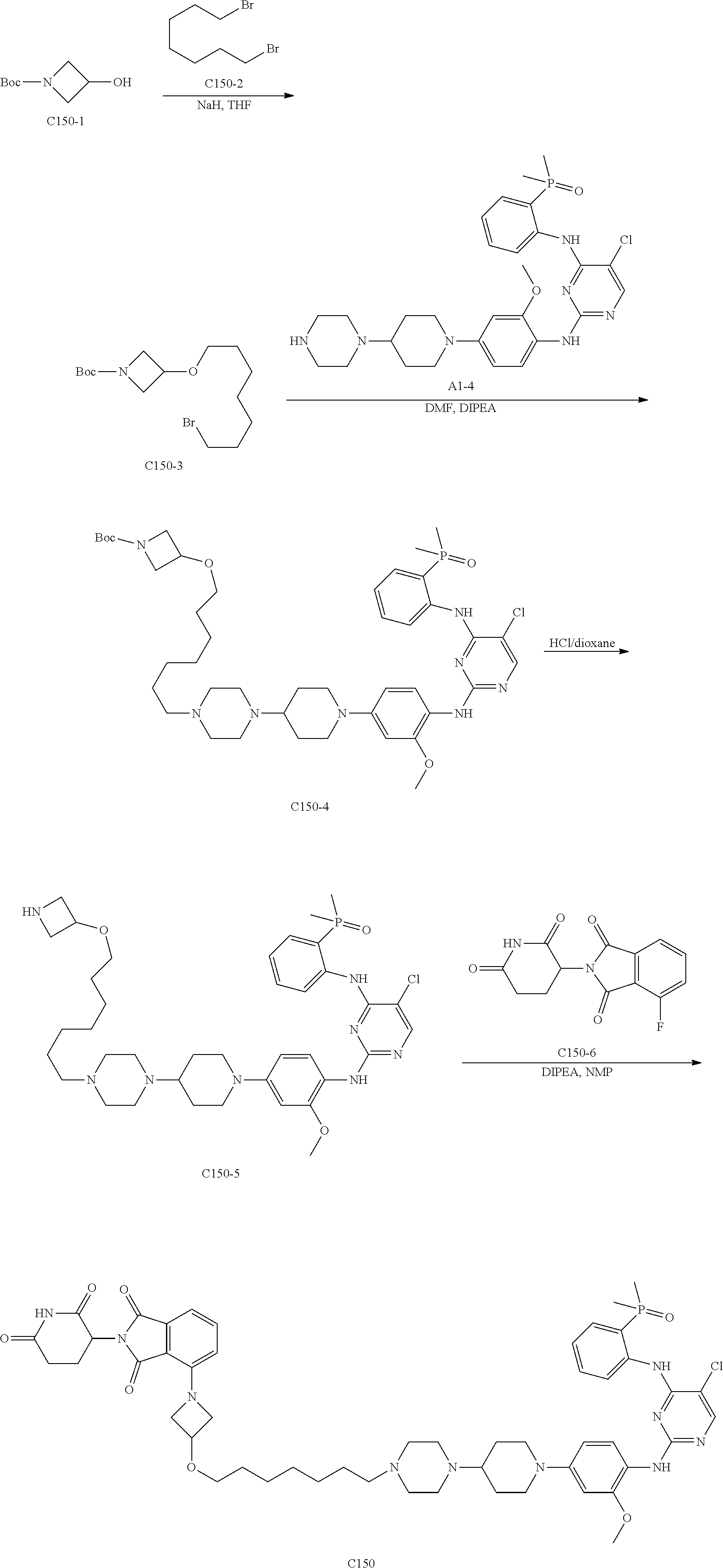

### Step 1

Compound C150-1 (470 mg, 2.72 mmol) was added to a 100 mL three-necked flask and dissolved in anhydrous tetrahydrofuran (6 mL). The mixture was cooled to 0° C. under nitrogen protection, and sodium hydride (120 mg, 2.98 mmol) was then added. The mixture was warmed to room temperature and reacted with stirring for 30 minutes. The mixture was cooled to 0° C. again, and compound C150-2 (842 mg, 3.26 mmol) was added. The mixture was reacted at room temperature for 3 h. The reaction process was monitored by TLC. The reaction was stopped until the raw materials were basically transformed. Ice water (3 g) was added to the mixture. The mixture was extracted twice with ethyl acetate (10 mL for each time). The organic phases were combined, washed with saturated brine (5 mL×2), dried with anhydrous sodium sulfate, and filtered. The filtrate was concentrated under reduced pressure to afford a crude product, which was separated by column chromatography to afford light-yellow solid C150-3, 100 mg, yield: 10.6%.

### Step 2

Compound C150-3 (35 mg, 0.1 mmol), compound A1-4 (57 mg, 0.1 mmol) and DIPEA (38.7 mg, 0.3 mmol) were added to a 50 mL single-necked flask and dissolved in DMF (2 mL). The mixture was heated to 70° C. and reacted for 2 h. The reaction process was monitored by LCMS. After the reaction was completed, the mixture was cooled, and ethyl acetate (10 mL) and water (5 mL) were added to the mixture. The layers were separated, and the aqueous layer was extracted once with ethyl acetate. The organic phases were combined, washed with saturated brine (5 mL×2), dried with anhydrous sodium sulfate, and filtered. The filtrate was concentrated under reduced pressure to afford a crude product, which was separated by Prep-TLC to afford light white solid C150-4, 35 mg, yield: 41.7%.

### Step 3

Compound C150-4 (30 mg, 0.035 mmol) and HCl/1,4-dioxane solution (5 mL, 4 mol/L) were added to a 25 mL single-necked flask. The mixture was reacted with stirring at room temperature for 2 h. The reaction process was monitored by TLC. After the reaction was completed, the solvent was concentrated under reduced pressure, and the residue was dissolved with dichloromethane (20 mL). Sodium bicarbonate solution was added to adjust the pH to 8-9. After separation, the dichloromethane solution was dried with anhydrous sodium sulfate, and filtered. The filtrate was concentrated under reduced pressure to afford light-yellow solid C150-5, 25 mg, yield: 99%. LCMS: [M+H]⁺=739.

### Step 4

Compound C150-5 (20 mg, 0.027 mmol), C150-6 (or A6-1) (8.8 mg, 0.032 mmol), DIPEA (10.4 mg, 0.081 mmol) and NMP (2 mL) were added to a 25 mL single-necked flask. The mixture was heated to 90° C. and reacted under nitrogen protection for 3 h. LCMS showed that a small amount of product was produced. The mixture was directly separated by prep-HPLC to afford white powder C150, 0.7 mg, yield: 2.6%. LCMS: [M+H]⁺=995.

### Preparation of 3-(4-((7-(4-(1-(4-((5-chloro-4-((2-(dimethylphosphoryl)phenyl)amino)pyrimidin-2-yl)amino)-3-methoxyphenyl)piperidin-4-yl)piperazin-1-yl)heptyl)oxy)-1-oxoisoindolin-2-yl)piperidine-2,6-dione (C153)

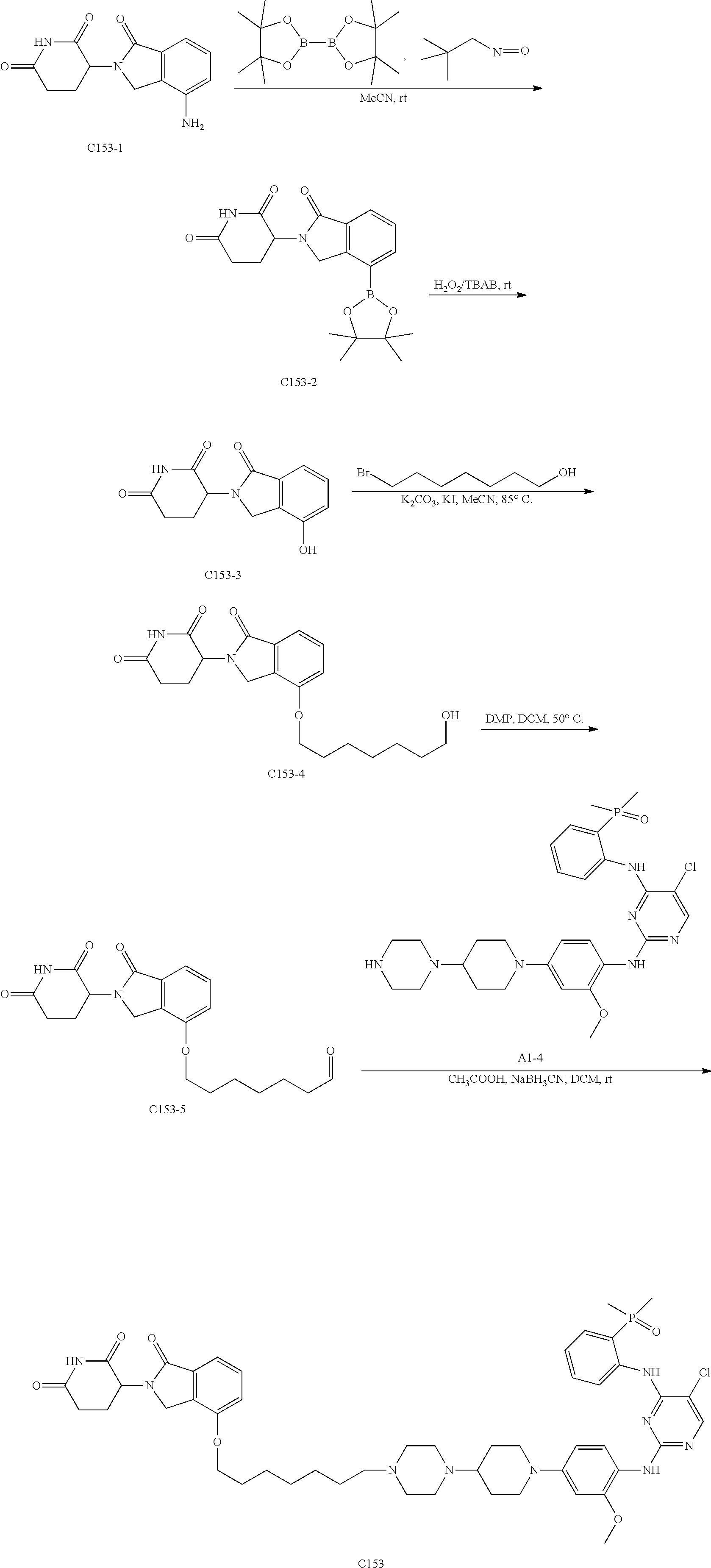

### Step 1

C153-1 (or A12-3) (2.59 g, 10.0 mmol) and bis(pinacolato)diboron (2.794 g, 11.0 mmol) were dispersed in 80.0 mL of anhydrous MeCN, and tert-butyl nitrite (1.545 g, 15.0 mmol) was added dropwise under nitrogen protection. After the dropwise addition, the mixture was reacted at room temperature for 4.0 h. The mixture was diluted with 80 mL of ethyl acetate, stirred for 5 minutes, and filtered with suction. The filtrate was concentrated under reduced pressure to afford a crude product, which was purified by Flash chromatography to afford light-yellow solid product C153-2 (2.1 g, 56.7%). LCMS: [M+H]⁺=371.

### Step 2

C153-2 (2.1 g, 5.68 mmol) was dispersed in 63.0 H₂O₂ (30%), and TBAB (548 mg, 1.70 mmol) was added with stirring. The mixture was reacted in air for 1 h. LCMS showed that the reaction was completed. The mixture was diluted with 150 mL of ethyl acetate, and stirred for 5 minutes. The organic layer was separated, and then the aqueous phase was extracted once. The organic layers were combined, and washed with saturated brine (50 mL×2). The organic layer was dried with anhydrous Na₂SO₄, and filtered with suction to remove the desiccant. The filtrate was concentrated under reduced pressure to afford a crude product, which was purified by Flash chromatography to afford light-yellow solid product C153-3 (0.71 g, 48.2%). LCMS: [M+H]+=261.

### Step 3

C153-3 (130 mg, 0.5 mmol), K₂CO₃ (103.5 mg, 0.75 mmol) and KI (41.5 mg, 0.25 mmol) were dispersed in 13.0 mL of anhydrous MeCN, and 7-bromo-1-heptanol (195 mg, 1.0 mmol) was added. The mixture was reacted at 85° C. for 1.5 h. The mixture was cooled to room temperature, and then filtered with suction to remove solid residue. The filtrate was concentrated under reduced pressure to afford a crude product. The crude product was purified by Prep-TLC to afford light-yellow solid product C153-4 (115 mg, 61.5%). LCMS: [M+H]⁺=375.

### Step 4

C153-4 (75 mg, 0.2 mmol) was dissolved in 10.0 mL of anhydrous DCM, and Dess-Martin reagent (127.5 mg, 0.3 mmol) was added. The mixture was reacted under reflux at 50° C. for 2 h. The mixture was cooled to room temperature, and diluted with 10 mL of DCM. 5 mL of saturated NaHCO₃ solution and 5 mL of saturated Na₂S₂O₃ solution were then added, and the mixture was stirred for 5 min. The organic layer was separated, washed with saturated NaCl solution (10 mL×1), dried with anhydrous Na₂SO₄, and filtered with suction to remove the desiccant. The filtrate was concentrated under reduced pressure to afford a crude product, which was purified by Prep-TLC to afford light-yellow solid product C153-5 (40 mg, 53%). LCMS: [M+H]+=373.

### Step 5

C153-5 (40 mg, 0.1075 mmol) and A1-4 (55.1 mg, 0.0968 mmol) were dissolved in 8.0 mL of anhydrous DCM. CH₃COOH (6.45 mg, 0.1075 mmol) was added dropwise under nitrogen protection. The mixture was stirred at room temperature for 30 min. Then NaBH₃CN (62.8 mg, 0.1613 mmol) was added. The mixture was reacted with stirring at room temperature for 2 h. The mixture was diluted with 30 mL of DCM, and then washed successively with saturated NH₄Cl solution (15 mL×1) and saturated NaCl solution (10 mL×1). The organic layer was dried with anhydrous Na₂SO₄, and filtered with suction to remove the desiccant. The filtrate was concentrated under reduced pressure to afford a crude product, which was purified by Prep-HPLC to afford white solid pure product C153 (16 mg, yield: 16.2%). LCMS: [M+H]+=926. ¹H-NMR: (400 MHz, DMSO-d₆) δ 11.163 (s, 1H), 10.962 (s, 1H), 8.480 (brs, 1H), 8.061 (d, J=13.2 Hz, 1H), 7.534-7.492 (m, 2H), 7.473-7.292 (m, 3H), 7.241-7.221 (m, 1H), 7.110 (t, J=7.2 Hz, 1H), 6.623 (d, J=2.4 Hz, 1H), 6.477 (dd, J=2.4, 8.8 Hz, 1H), 5.127 (dd, J=5.2, 13.6 Hz, 1H), 4.390 (d, J=17.6 Hz, 1H), 4.244 (d, J=17.2 Hz, 1H), 4.128 (t, J=6.4 Hz, 2H), 3.758 (s, 3H), 3.720-3.690 (m, 2H), 3.300-3.277 (m, 1H), 2.954-2.864 (m, 1H), 2.690-2.558 (m, 4H), 2.328-2.204 (m, 7H), 2.010-1.974 (m, 2H), 1.855-1.745 (m, 10H), 1.549-1.237 (m, 12H).

### Preparation of 4-((7-(4-(1-(4-((5-bromo-4-((5-(dimethylphosphoryl)quinoxalin-6-yl)amino)pyrimidin-2-yl)amino)-5-methoxy-2-methylphenyl)piperidin-4-yl)piperazin-1-yl)heptyl)oxy)-2-(2,6-dioxopiperidin-3-yl)isoindoline-1,3-dione (C158)

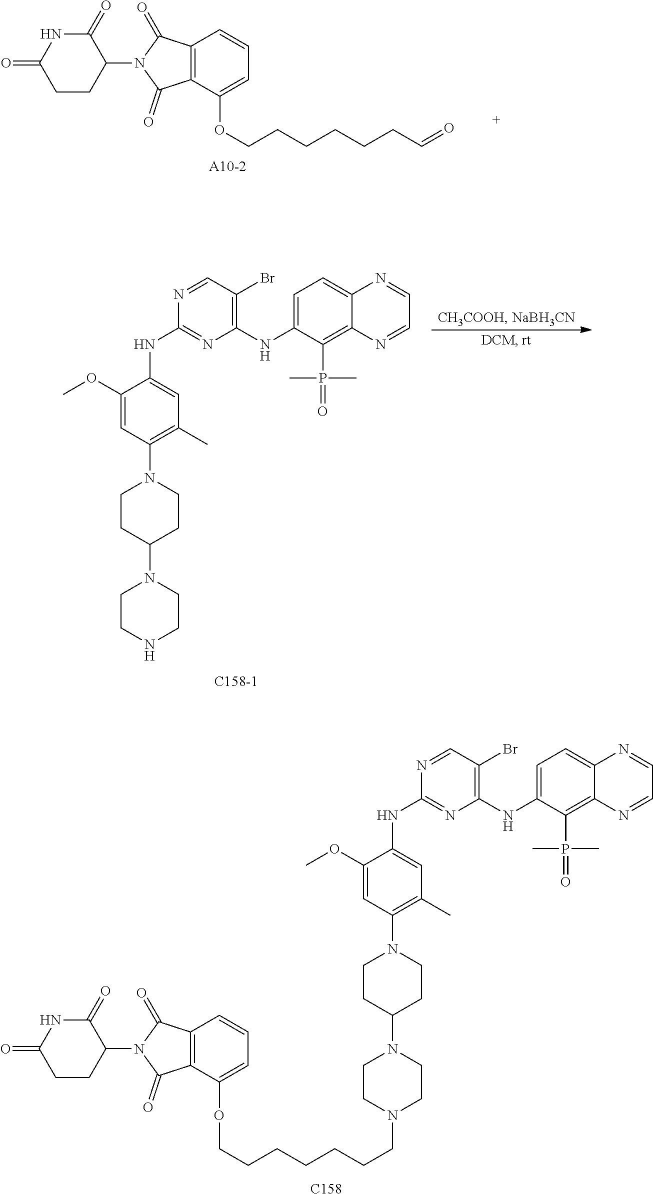

A10-2 (46.3 mg, 0.12 mmol) and C158-1 (67.9 mg, 0.1 mmol) were dissolved in 10.0 mL of anhydrous DCM. CH₃COOH (6.0 mg, 0.1 mmol, dissolved in 0.5 mL of DCM) was added under nitrogen protection. The mixture was stirred at room temperature for 1 h. Then solid NaBH₃CN (15.7 mg, 0.25 mmol) was added and the mixture was further reacted at room temperature for 2 h. LCMS showed that the reaction was completed. The mixture was diluted with 20 mL of DCM, and then washed successively with saturated NH₄Cl (1×15 mL) and saturated brine (1×15 mL). The organic layer was dried with anhydrous Na₂SO₄, and filtered with suction. The filtrate was concentrated under reduced pressure to afford a crude product. The crude product was purified by Prep-HPLC to afford yellow solid pure product C158, 36 mg, yield: 34.3%. LCMS: [M+H]⁺=1050. ¹H-NMR: (400 MHz, DMSO-d₆) δ 11.749 (s, 1H), 11.096 (s, 1H), 8.887-8.849 (m, 3H), 8.341 (s, 1H), 8.288 (s, 1H), 7.948 (d, J=9.6 Hz, 1H), 7.841 (t, J=7.2 Hz, 1H), 7.531 (d, J=8.8 Hz, 1H), 7.467 (d, J=7.2 Hz, 1H), 7.406 (s, 1H), 6.767 (s, 1H), 5.102 (dd, J=5.2, 12.8 Hz, 1H), 4.583 (s, 6H), 4.236 (t, J=6.4 Hz, 3H), 3.789 (s, 3H), 3.198-2.700 (m, 9H), 2.625-32.532 (m, 3H), 2.111 (s, 3H), 2.049 (s, 4H), 2.013 (s, 3H), 1.800-1.647 (m, 5H), 1.495-1.376 (m, 6H), 1.238 (s, 2H).

### Preparation of 3-(4-(1-(5-(4-(1-(4-((5-chloro-4-((2-(dimethylphosphoryl)phenyl)amino)pyrimidin-2-yl)amino)-3-methoxyphenyl)piperidin-4-yl)piperazin-1-yl)pentyl)-1H-pyrazol-4-yl)-1-oxoisoindolin-2-yl)piperidine-2,6-dione (C162)

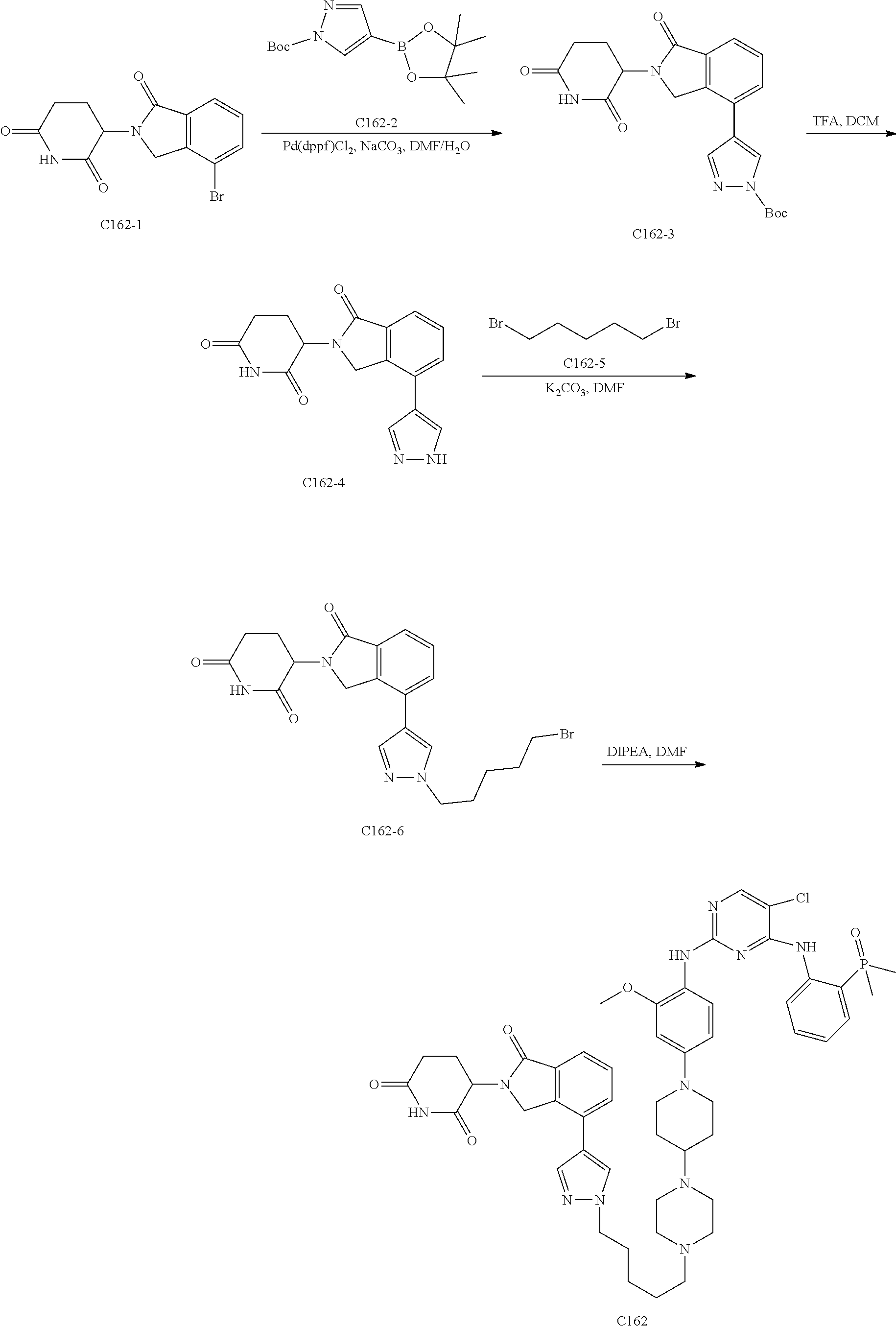

### Step 1

Compound C162-1 (or C1-1) (300 mg, 1 mmol), compound C162-2 (440 mg, 1.5 mmol), Pd(dppf)Cl₂ (73.1 mg, 0.1 mmol) and anhydrous sodium carbonate (414 mg, 3 mmol) were added to a 50 mL single-necked flask, and then DMF (10 mL) and water (2 mL) were added. The mixture was degassed and deoxidized with nitrogen gas, and heated to 90° C. in a microwave reactor under nitrogen protection for 1 h. After the mixture was cooled, water (30 mL) was added and the mixture was extracted with ethyl acetate for three times (20 mL for each time). The organic phases were combined, washed with brine (25 mL×3), dried with anhydrous sodium sulfate, and filtered. The filtrate was concentrated under reduced pressure to afford a crude product. The crude product was separated by column chromatography to afford light white solid C162-3, 140 mg, yield: 34%.

### Step 2

Compound C162-3 (70 mg, 0.17 mmol) and dichloromethane (3 mL) were added to a 25 mL single-necked flask and dissolved with stirring. Then, trifluoroacetate (2 mL) was added. The mixture was reacted with stirring at room temperature for 1 h. The reaction process was monitored by TLC. After the reaction was completed, the mixture was concentrated under reduced pressure to remove dichloromethane and most of trifluoroacetate, and dissolved with dichloromethane (10 mL). Sodium bicarbonate solution was added to adjust the pH to 8-9. After separation, the dichloromethane solution was dried with anhydrous sodium sulfate, and filtered. The filtrate was concentrated under reduced pressure to afford light-yellow solid C162-4, 51 mg, yield: 96%. LCMS: [M+H]⁺=311.

### Step 3

Compound C162-4 (50 mg, 0.16 mmol), compound C162-5 (36.8 mg, 0.16 mmol) and potassium carbonate (66.24 mg, 0.48 mmol) were added to a 25 mL single-necked flask, and then DMF (3 mL) was added. The mixture was stirred and heated to 45° C. to react overnight. The reaction process was monitored by LCMS. After the reaction was completed, the mixture was cooled. Water (10 mL) was added and the mixture was extracted with ethyl acetate for three times (5 mL for each time). The organic phases were combined, washed with brine (5 mL×3), dried with anhydrous sodium sulfate, and filtered. The filtrate was concentrated under reduced pressure to afford a crude product. The crude product was separated by preparative thin layer chromatography to afford light-yellow waxy solid C162-6, 22 mg, yield: 30.1%. LCMS: [M+H]+=459, 461.

### Step 4

Compound C162-6 (20 mg, 0.053 mmol), compound A1-4 (30.5 mg, 0.053 mmol) and diisopropylethylamine (20.5 mg, 0.159 mmol) were added to a 25 mL single-necked flask, and then DMF (2 mL) was added. The mixture was dissolved with stirring, heated to 70° C. and reacted for 2 h. The reaction process was monitored by LCMS. After the reaction was completed, the mixture was cooled and concentrated under reduced pressure to afford a crude product. The crude product was separated by preparative high performance liquid chromatography to afford white solid C162, 9 mg, yield: 17.9%. LCMS: [M+H]+=948.

### Preparation of 3-(5-(7-(4-(1-(4-((5-bromo-4-((5-(dimethylphosphoryl)quinoxalin-6-yl)amino)pyrimidin-2-yl)amino)-5-methoxy-2-methylphenyl)piperidin-4-yl)piperazin-1-yl)hept-1-yn-1-yl)-1-oxoisoindolin-2-yl)piperidine-2,6-dione (C163)

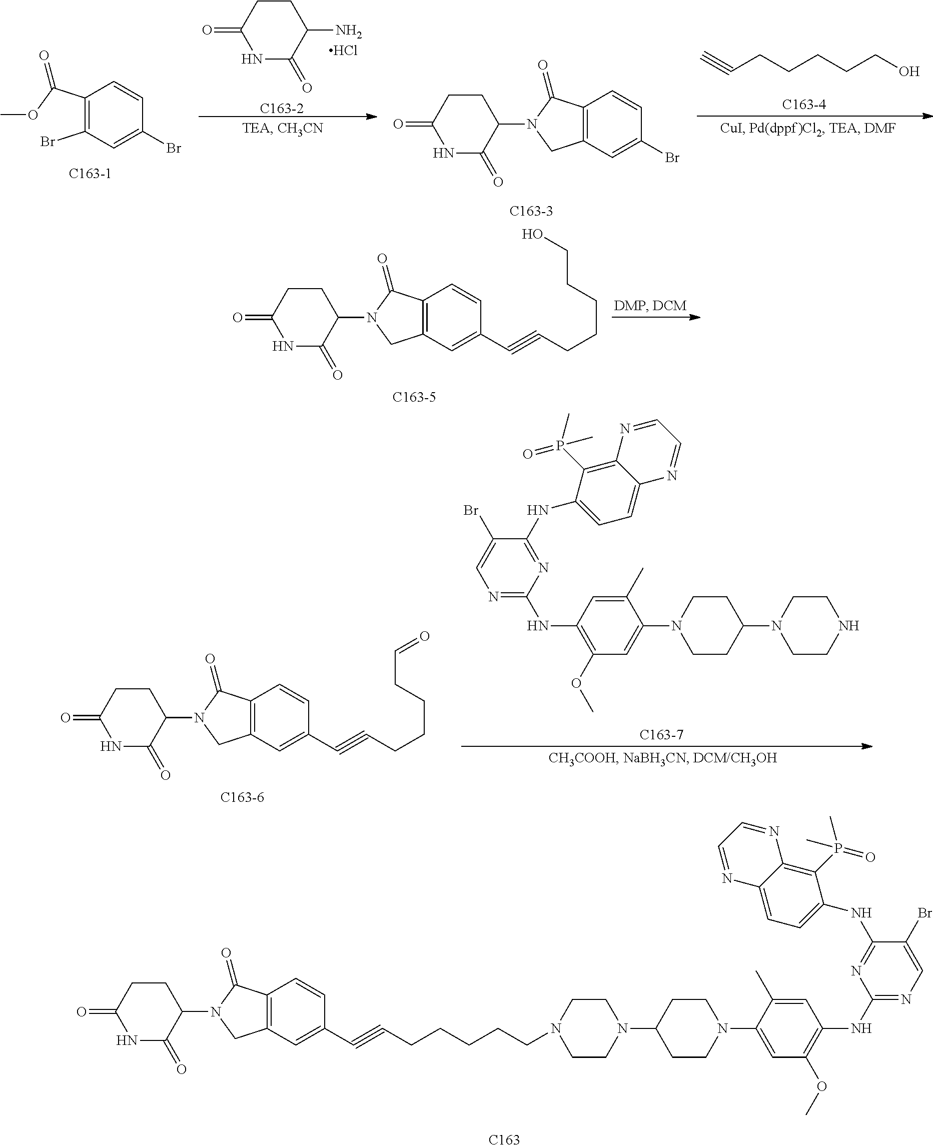

### Step 1

Compound C163-1 (5 g, 16.35 mmol), compound C163-2 (3.49 g, 21.25 mmol), and triethylamine (2.15 g, 21.25 mmol) were dissolved in 80 mL of acetonitrile. The mixture was stirred at 80° C. under nitrogen protection overnight. The mixture was cooled to room temperature, and filtered. Solids were rinsed with acetonitrile. Mother liquor was concentrated, and washed with a small amount of acetonitrile. Solids were combined and dried to afford white solid compound C163-3, which was directly used in the next step (7.03 g, yield: 84.4%). LCMS: [M+H]⁺=323, 325.

### Step 2

Compound C163-3 (322 mg, 1 mmol), compound C163-4 (280 mg, 2.5 mmol), cuprous iodide (38 mg, 0.2 mmol), Pd(dppf)Cl₂ (280 mg, 0.4 mmol) and triethylamine (30.3 mg, 3 mmol) were dissolved in 10 mL of anhydrous DMF. The mixture was reacted with stirring at 70° C. under nitrogen protection overnight. The solvent was removed under reduced pressure. The crude product was purified by thin layer chromatography to afford off-white solid compound C163-5 (135 mg, yield: 38.1%). LCMS: [M+H]⁺=355.

### Step 3

Compound C163-5 (45 mg, 0.127 mmol) and Dess-Martin reagent (162 mg, mmol) were dissolved in 20 mL of dichloromethane and stirred at 50° C. for 2 h. The mixture was cooled to room temperature, and quenched with saturated sodium bicarbonate aqueous solution. The organic phase was separated, and the aqueous phase was extracted with dichloromethane for three times. The organic phases were combined, dried with anhydrous sodium sulfate, and filtered. The filtrate was concentrated, and the crude product was purified by TLC to afford a white solid compound C163-6 (26.7 mg, yield: 60.7%). LCMS: [M+H]⁺=353.

### Step 4

Compound C163-6 (26.7 mg, 0.076 mmol) and C163-7 (or C158-1) (51.5 mg, 0.076 mmol) were dissolved in 3 mL of dichloromethane/methanol (2/1), and 1 drop of glacial acetic acid was added dropwise. The mixture was stirred at room temperature under nitrogen protection for 1 h. Then sodium cyanobohydride (4.8 mg, 0.076 mmol) was added and the mixture was further stirred for 1 h. The solvent was removed under reduced pressure below 40° C., and the crude product was purified by Flash chromatography to afford off-white solid compound C163 (15 mg, yield: 19.5%). LCMS: [M+H]⁺=1016, 1018.

### Preparation of 4-((7-(4-(1-(4-((5-chloro-4-((2-(dimethylphosphoryl)phenyl)amino)pyrimidin-2-yl)amino)-3-methoxyphenyl)piperidin-4-yl)piperazin-1-yl)heptyl-7,7-dideuterium)oxy)-2-(2,6-dioxopiperidin-3-yl)isoindoline-1,3-dione (C164)

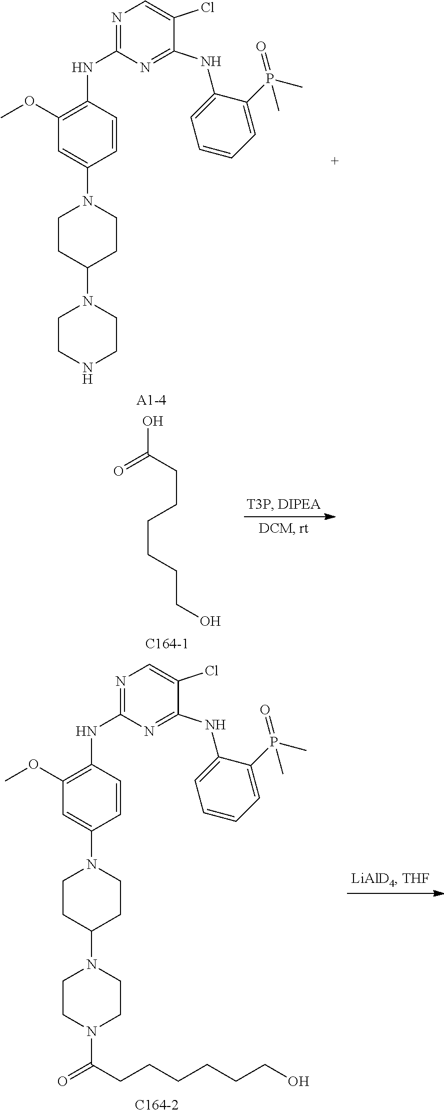

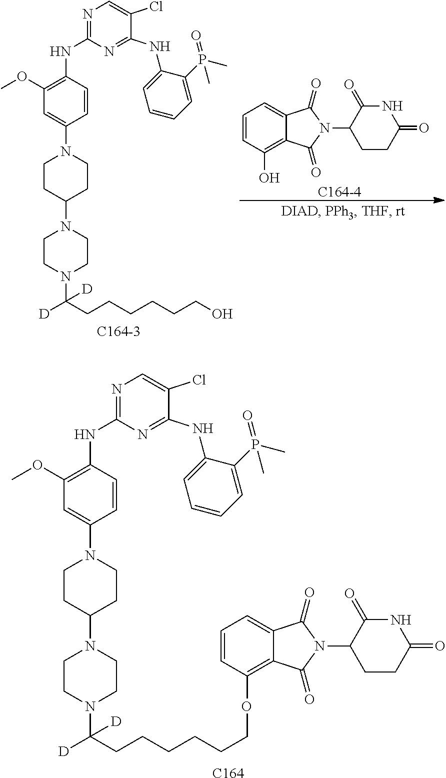

### Step 1

A1-4 (100 mg, 0.176 mmol) and C164-1 (30.8 mg, 0.211 mmol) were dispersed in 10.0 mL of anhydrous DCM, and T3P (224 mg, 0.352 mmol) and DIPEA (90.8 mg, 0.704 mmol) were added successively under nitrogen protection. The mixture was reacted at room temperature for 2 h. The mixture was diluted with 20 mL of dichloromethane, and then washed successively with saturated NH₄Cl (10 mL×1) and saturated NaCl (10 mL×1). The organic layer was dried with anhydrous MgSO₄, and filtered with suction. The filtrate was concentrated under reduced pressure to afford a crude product. The crude product was purified by Prep-TLC to afford yellow solid product C164-2 (45 mg, 36.7%). LCMS: [M+H]⁺=698.

### Step 2

C164-2 (45 mg, 0.065 mmol) was dissolved in 10.0 mL of anhydrous THF. LiAlD₄ (27.1 mg, 0.65 mmol) was added under nitrogen protection. The mixture was reacted at 70° C. for 2.0 h. LCMS showed that the reaction was completed. The mixture was cooled to 0° C., quenched with 1.0 mL of H₂O, and then 5 drops of 1 mol/L NaOH solution was added to the mixture and stirred for 5 min. The organic layer was separated and concentrated under reduced pressure to afford crude C164-3 (35 mg, 77.7%). The crude product was directly used in the next step. LCMS: [M+H]⁺=686.

### Step 3

C164-3 (35 mg, 0.051 mmol), C164-4 (or A2-1) (15.4 mg, 0.056 mmol) and PPh₃ (20 mg, 0.077 mmol) were dissolved in 6.0 mL of anhydrous THF. DIAD (15.4 mg, 0.077 mmol) was added under nitrogen protection. The mixture was reacted at room temperature for 2 h. LCMS showed that the reaction was completed. The mixture was concentrated under reduced pressure to afford a crude product, which was purified by Prep-HPLC to afford light-yellow solid product C164 (10 mg, yield: 20.8%). LCMS: [M+H]⁺=942.

### Preparation of 3-(4-(7-(4-(1-(4-((5-chloro-4-((2-(dimethylphosphoryl)phenyl)amino)pyrimidin-2-yl)amino)-3-methoxyphenyl)piperidin-4-yl)piperazin-1-yl)hept-1-yn-1-yl)-5-fluoro-1-oxoisoindolin-2-yl)piperidine-2,6-dione (C166)

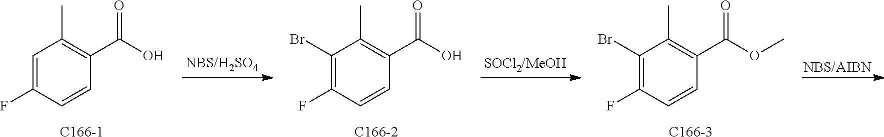

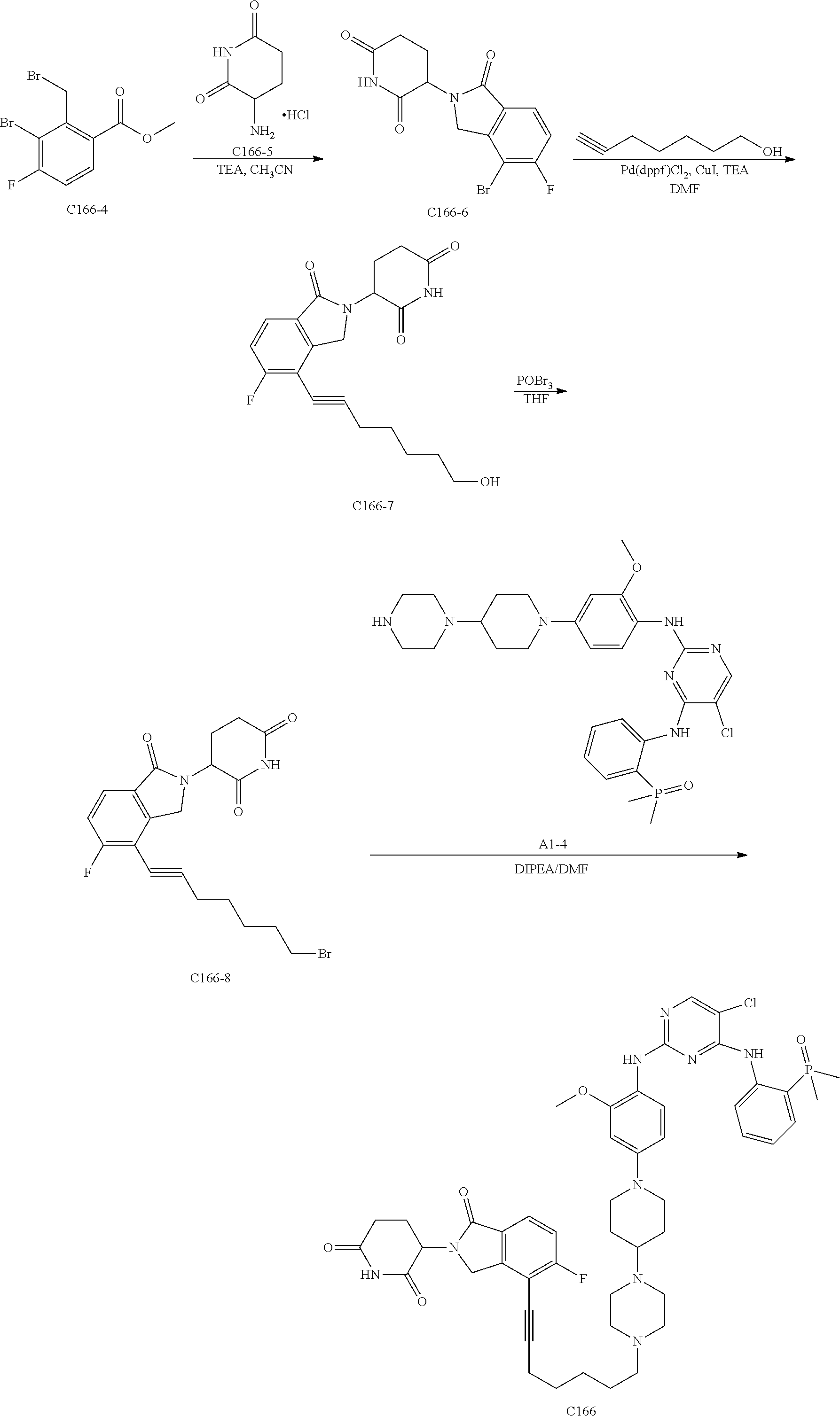

### Step 1

Compound C166-1 (10 g, 64.87 mmol) was added to a 500 mL single-necked flask, and then sulfuric acid (200 mL) was added. The mixture was dissolved with stirring, and then NBS (10.4 g, 58.44 mmol) was added. The mixture was reacted with stirring at room temperature for 5 h. The reaction process was monitored by LCMS. After the reaction was completed, the mixture was added to ice water (1000 g) to quench the reaction. White solids were precipitated, and collected by filtration. The filter cake was washed three times with water, 200 mL for each time, to afford a white solid containing water, which was vacuum-dried at 50° C. to afford 12 g of a mixture of white powder C166-2, which was directly used in the next step.

### Step 2

The mixture of compound C166-2 (6 g, 25.75 mmol) was added to a 250 mL single-necked flask and dissolved in methanol (100 mL). The mixture was cooled to 0° C., and then thionyl chloride (7.67 g, 64.37 mmol) was added dropwise. After the dropwise addition, the mixture was heated to reflux and reacted for 2 h. The reaction process was monitored by TLC. The reaction was stopped until the raw materials were basically transformed. The mixture was concentrated under reduced pressure to remove most of solvent and thionyl chloride, and then ice water was added to the concentrated solution. The mixture was extracted with ethyl acetate (100 mL×3). The organic phases were combined, washed successively with saturated sodium bicarbonate solution, water and brine (50 mL×2), dried with anhydrous sodium sulfate, and filtered. The filtrate was concentrated under reduced pressure to afford 5.2 g of light-yellow oily product, yield: 81.7%, which was directly used in the next step.

### Step 3

The mixture of compound C166-3 (3 g, 12.1 mmol) was added to a 250 mL single-necked flask, and dissolved with chloroform (100 mL). NBS (2.16 g, 12.1 mmol) and AIBN (196.8 mg, 1.21 mmol) were then added successively. The mixture was stirred and heated to reflux for 5 h. The reaction process was monitored by TLC. The reaction was stopped until the raw materials were basically transformed into the product. The mixture was cooled, and then the insolubles were removed by filtration. The mother liquor was washed with water for three times (20 mL for each time), dried with anhydrous sodium sulfate, and filtered. The filtrate was concentrated under reduced pressure to afford a crude product, which was separated by column chromatography to afford 2.2 g of colorless oily product C166-4.

### Step 4

Compound C166-4 (2 g, 12.24 mmol), compound C166-5 (4 g, 12.24 mmol), acetonitrile (100 mL) and triethylamine (2.56 mL, 36.72 mmol) were successively added to a 250 mL single-necked flask. The mixture was heated to reflux and reacted overnight. The mixture was cooled, and then solids were precipitated. The precipitation was further continued for 5 h. Then the mixture was filtered to afford a light white solid, which was washed with acetonitrile (20 mL×2), and vacuum-dried at 40° C. to afford 700 mg of light white powder C166-6. Yield: 16.8%. LCMS: [M+H]⁺=341, 343.

### Step 5

Compound C166-6 (700 mg, 2.05 mmol), hept-6-yn-1-ol (392 mg, 3.5 mmol), Pd(PPh₃)₂Cl₂ (490 mg, 0.35 mmol), cuprous iodide (66.8 mg, 0.35 mmol) and triethylamine (530 mg, 5.25 mmol) were added to a 100 mL three-necked flask, and then DMF (20 mL) was added. The mixture was dissolved, then heated to 70° C. and reacted under nitrogen protection for 5 h. The reaction process was monitored by LCMS. After the reaction was completed, the mixture was cooled and concentrated under reduced pressure to afford a crude product, which was separated by column chromatography to afford 170 mg of light-yellow solid C166-7. Yield: 22.3%. LCMS: [M+H]⁺=373.2.

### Step 6

Compound C166-7 (170 mg, 0.46 mmol) was added to a 50 mL three-necked flask equipped with a condenser and dissolved in THE (3 mL). Phosphorus oxybromide (170 mg, 0.59 mmol) was then added. The mixture was heated to 70° C. and reacted for 2 h. The reaction process was monitored by LCMS. After the reaction was completed, the mixture was cooled, and ethyl acetate (10 mL) was added. The mixture was washed twice with saturated sodium bicarbonate solution, 5 mL for each time, then washed with brine (5 mL×2), dried with anhydrous sodium sulfate, and filtered. The filtrate was concentrated under reduced pressure to afford a crude product. The crude product was separated by preparative thin layer chromatography to afford 39 mg of light white solid C166-8. Yield: 19.5%. LCMS: [M+H]⁺=435, 437.

### Step 7

Compound C166-8 (40 mg, 0.11 mmol), A1-4 (61.2 mg, 0.11 mmol), diisopropylethylamine (41.7 mg, 0.33 mmol) and DMF (2 mL) were added to a 25 mL single-necked flask equipped with a condenser. The mixture was heated to 70° C. and reacted for 3 h. The reaction process was monitored by LCMS. The reaction was stopped until the raw materials were basically transformed into the title product. The mixture was cooled and concentrated under reduced pressure to afford a crude product, which was separated by prep-HPLC to afford 10 mg of white powder C166. Yield: 10%. LCMS: [M+H]⁺=924.

### Preparation of 3-(4-(7-(4-(1-(4-((5-chloro-4-((2-(dimethylphosphoryl)phenyl)amino)pyrimidin-2-yl)amino)-3-methoxyphenyl)piperidin-4-yl)piperazin-1-yl)hept-1-yn-1-yl)-6-fluoro-1-oxoisoindolin-2-yl)piperidine-2,6-dione (C167)

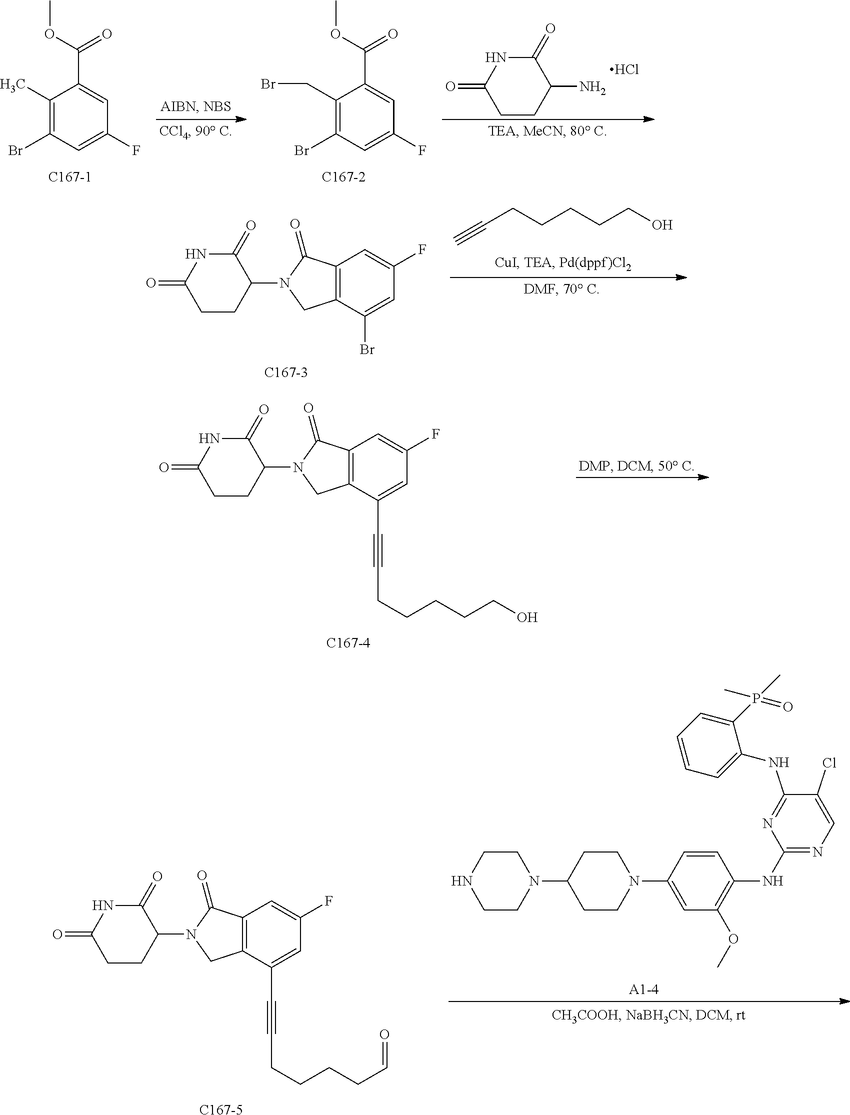

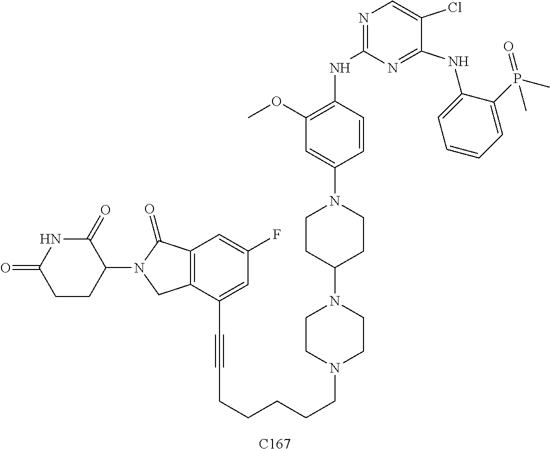 [SMILES: COc1cc(N2CCC(N3CCN(CCCCCC#Cc4cc(F)cc5c4CN(C4CCC(=O)NC4=O)C5=O)CC3)CC2)ccc1Nc1ncc(Cl)c(Nc2ccccc2P(C)(C)=O)n1 ; "C167"]

### Step 1

C167-1 (1.0 g, 4.06 mmol) was dissolved in 10.0 mL of CCl₄. NBS (1.1 g, 6.1 mmol) and AIBN (266.5 mg, 1.624 mmol) were added under nitrogen protection. The mixture was heated to 90° C., and reacted under reflux for 20 h. LCMS showed that there was no raw material remaining. The mixture was cooled to room temperature, and then filtered with suction. The filtrate was concentrated under reduced pressure to afford a crude product, which was purified by Flash chromatography to afford 1.2 g of light-yellow oily product C167-2 (which became solid upon standing).

### Step 2

C167-2 (1.2 g, 3.69 mmol) and 3-aminopiperidine-2,6-dione hydrochloride (787 mg, 4.80 mmol) were dispersed in 25.0 mL of anhydrous MeCN, and TEA (485 mg, 4.80 mmol) was added. The mixture was heated to 80° C., and reacted under reflux for 16 h. LCMS showed that the reaction was completed. The mixture was cooled to room temperature, and then filtrated with suction. The filter cake was washed with MeCN for three times, and dried with baking to afford product C167-3 (1.0 g, yield: 80%). LCMS: [M+H]⁺=341, 343.

### Step 3

C167-3 (340 mg, 1.0 mmol), CuI (38 mg, 0.2 mmol) and Pd(dppf)Cl₂ (292.4 mg, 0.4 mmol) were dispersed in 17.0 mL of anhydrous DMF. Hept-6-yn-1-ol (281 mg, 2.5 mmol) and TEA (303 mg, 3.0 mmol) were added successively under N₂ protection. The mixture was heated to 70° C. and reacted for 16 h. LCMS showed that the reaction was completed. The mixture was cooled to room temperature, diluted with 80 mL of ethyl acetate, and then washed successively with saturated NH₄Cl (2×40 mL), H₂O (2×40 mL) and saturated brine (2×40 mL). The organic layer was dried with anhydrous sodium sulfate, and filtered with suction. The filtrate was concentrated under reduced pressure to afford a crude product, which was purified by RP-Flash chromatography to afford light-yellow solid product C167-4 (95 mg, 25.5%). LCMS: [M+H]⁺=373.

### Step 4

C167-4 (90 mg, 0.242 mmol) was dispersed in 25.0 mL of anhydrous DCM. Dess-Martin reagent (154 mg, 0.363 mmol) was added under N₂ protection. The mixture was heated to 50° C., and reacted under reflux for 2.0 h. TLC showed that the reaction was completed. The mixture was cooled to room temperature, and then 10 mL of saturated NaHCO₃ solution and 10 mL of saturated Na₂S₂O₃ solution were added. The mixture was stirred at room temperature for 5 min. The organic layer was separated, dried with anhydrous sodium sulfate, and filtered with suction. The filtrate was concentrated under reduced pressure to afford a crude product. The crude product was purified by Prep-TLC to afford light-yellow solid product C167-5 (55 mg, 60%). LCMS: [M+H]⁺=371.

### Step 5

C167-5 (30.0 mg, 0.081 mmol) and A1-4 (46.1 mg, 0.081 mmol) were dissolved in 5.5 mL of anhydrous DCM. CH₃COOH (4.86 mg, 0.081 mmol, dissolved in 0.5 mL of DCM) was added under nitrogen protection. The mixture was stirred at room temperature for 0.5 h. Then solid NaBH₃CN (10.2 mg, 0.162 mmol) was added. The mixture was further reacted at room temperature for 2 h. LCMS and TLC showed that the reaction was completed. The mixture was diluted with 20 mL of DCM, and then washed successively with saturated NH₄Cl (1×15 mL) and saturated brine (1×15 mL). The organic layer was dried with anhydrous Na₂SO₄, and filtered with suction. The filtrate was concentrated under reduced pressure to afford a crude product, which was purified by Prep-HPLC to afford 25 mg of white solid pure product. Yield: 33.5%. LCMS: [M+H]⁺=924. ¹H-NMR: (400 MHz, DMSO-d₆) δ 11.190 (s, 1H), 11.051 (s, 1H), 8.490 (br s, 1H), 8.098 (s, 1H), 8.066 (s, 1H), 7.563-7.507 (m, 3H), 7.397-7.315 (m, 2H), 7.110 (t, J=6.4 Hz, 1H), 6.628 (d, J=2.4 Hz, 1H), 6.479 (dd, J=2.4 Hz, 8.4 Hz, 1H), 5.182 (dd, J=5.2 Hz, 13.2 Hz, 1H), 4.462 (dd, J=17.2 Hz, 60.0 Hz, 2H), 3.758 (s, 3H), 3.725 (d, J=12.8 Hz, 2H), 2.967-2.876 (m, 2H), 2.672-2.503 (m, 3H), 2.501-2.397 (m, 6H), 2.398-2.247 (m, 3H), 2.032-1.974 (m, 1H), 1.832-1.748 (m, 12H), 1.5615-1.527 (m, 2H), 1.498-1.436 (m, 6H).

### Preparation of 3-(4-(3-(2-(4-(1-(4-((5-bromo-4-((5-(dimethylphosphoryl)quinoxalin-6-yl)amino)pyrimidin-2-yl)amino)-5-methoxy-2-methylphenyl)piperidin-4-yl)piperazin-1-yl)ethoxy)prop-1-yn-1-yl)-1-oxoisoindolin-2-yl)piperidine-2,6-dione (C169)

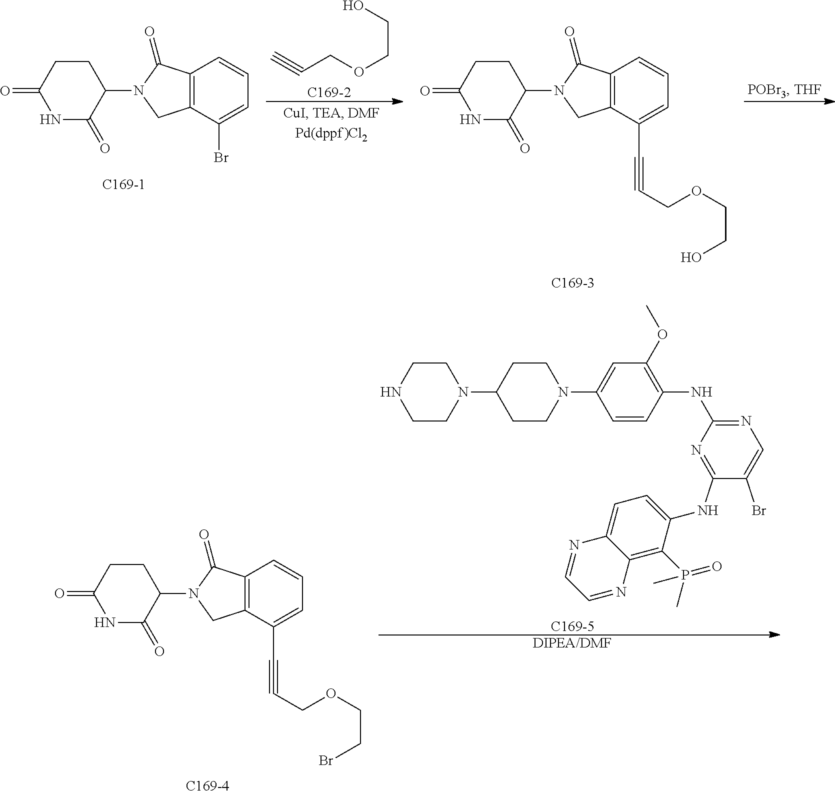

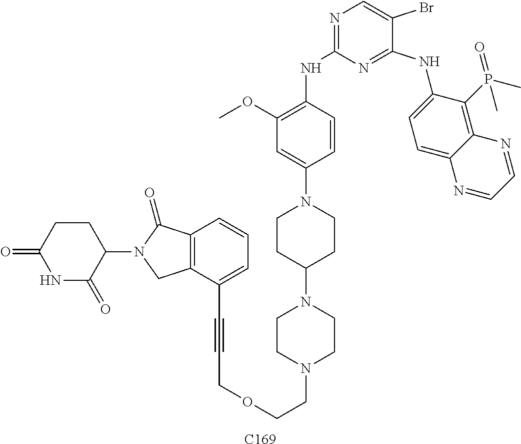 [SMILES: COc1cc(N2CCC(N3CCN(CCOCC#Cc4cccc5c4CN(C4CCC(=O)NC4=O)C5=O)CC3)CC2)ccc1Nc1ncc(Br)c(Nc2ccc3nccnc3c2P(C)(C)=O)n1 ; "C169"]

### Step 1

Compound C169-1 (or C1-1) (309 mg, 1 mmol), compound C169-2 (200 mg, 2 mmol), Pd(dppf)Cl₂ (280 mg, 0.4 mmol), cuprous iodide (38.2 mg, 0.2 mmol) and triethylamine (303 mg, 3 mmol) were added to a 100 mL single-necked flask, and then DMF (20 mL) was added. After the mixture was dissolved, the atmosphere was replaced with nitrogen gas for three times to remove oxygen. The mixture was heated to 70° C. and reacted under nitrogen protection for 5 h. The reaction process was monitored by LCMS. After the reaction was completed, the mixture was cooled and concentrated under reduced pressure to afford a crude product, which was separated by column chromatography to afford light-yellow solid C169-3, 70 mg, yield: 20.3%. LCMS: [M+H]⁺=343.

### Step 2

Compound C169-3 (70 mg, 0.2 mmol) was added to a 50 mL three-necked flask equipped with a condenser and dissolved in THE (2 mL). Phosphorus oxybromide (170 mg, 0.59 mmol) was then added. The mixture was heated to 70° C. and reacted for 2 h. The reaction process was monitored by LCMS. After the reaction was completed, the mixture was cooled, and ethyl acetate (10 mL) was added. The mixture was washed with saturated sodium bicarbonate solution (5 mL×2), then washed with brine (5 mL×2), dried with anhydrous sodium sulfate, and filtered. The filtrate was concentrated under reduced pressure to afford a crude product. The crude product was separated by preparative thin layer chromatography to afford 24 mg of light white solid C169-4. Yield: 29.7%. LCMS: [M+H]⁺=405, 407.

### Step 3

Compound C169-4 (12 mg, 0.029 mmol), compound C169-5 (or C158-1) (19.3 mg, 0.029 mmol), diisopropylethylamine (11.2 mg, 0.087 mmol) and DMF (1 mL) were added to a 25 mL single-necked flask equipped with a condenser. The mixture was heated to 70° C. and reacted for 3 h. The reaction process was monitored by LCMS. After the reaction was completed, the mixture was cooled and concentrated under reduced pressure to afford a crude product, which was separated by prep-HPLC to afford 10 mg of white powder C169. Yield: 34.5%. LCMS: [M+H]⁺=1004.

### Preparation of 4-(7-(9-(4-((5-bromo-4-((5-(dimethylphosphoryl)quinoxalin-6-yl)amino)pyrimidin-2-yl)amino)-5-methoxy-2-methylphenyl)-3,9-diazaspiro[5.5]undecan-3-yl)hept-1-yn-1-yl)-2-(2,6-dioxopiperidin-3-yl)isoindoline-1,3-dione (C171)

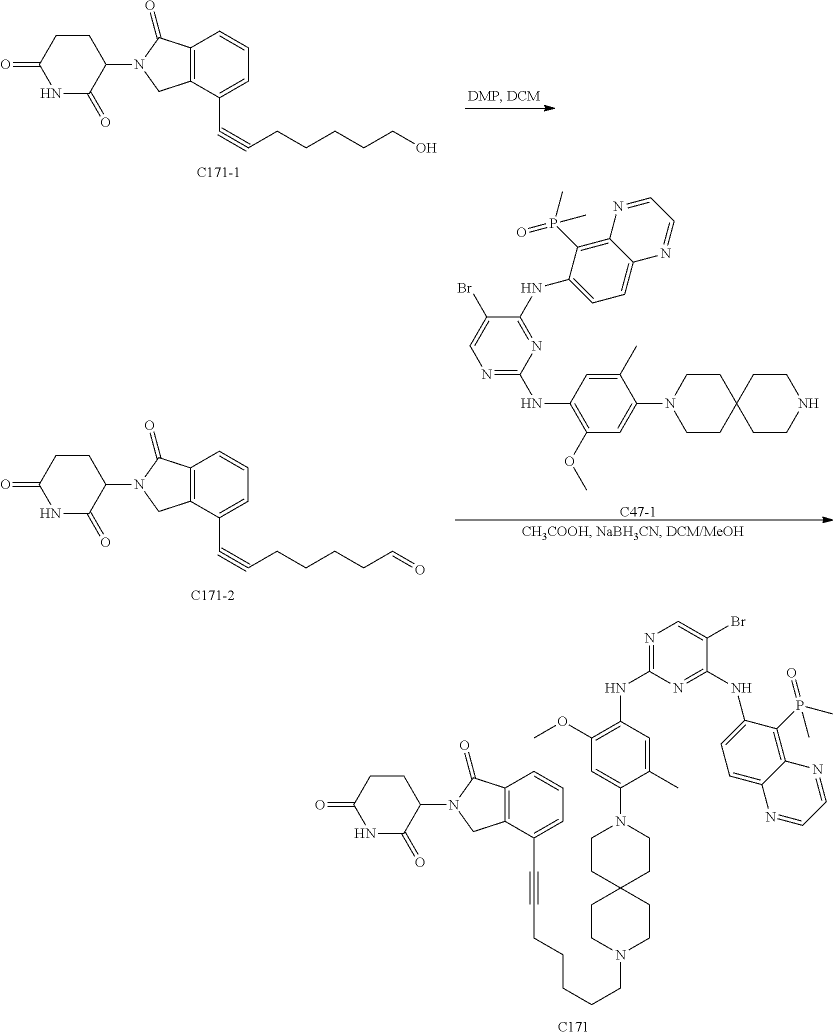

### Step 1

Compound C171-1 (or C148-3) (56.6 mg, 0.154 mmol) and Dess-Martin reagent (196 mg, 0.461 mmol) were dissolved in dichloromethane (20 mL). The mixture was reacted with stirring at 50° C. for 2 h. The mixture was cooled to room temperature, and quenched with saturated sodium bicarbonate aqueous solution. The layers were separated, and the aqueous phase was extracted with dichloromethane for three times. The organic phases were combined, washed with saturated brine (15 mL×2), dried with anhydrous sodium sulfate, and filtered. The solvent was removed under reduced pressure. The crude product was separated by column chromatography to afford compound C171-2 (45 mg, yield: 80.4%). LCMS: [M+H]⁺=367.

### Step 2

Compound C171-2 (45 mg, 0.12 mmol) and compound C47-1 (82 mg, 0.12 mmol) were dissolved in dichloromethane and methanol (2/1) (3 mL), and 1 drop of glacial acetic acid was added. The mixture was stirred at room temperature under nitrogen protection for 1 h. Then sodium cyanobohydride (7.8 mg, 0.12 mmol) was added. The mixture was further stirred for 1 h. The mixture was concentrated to remove the solvent. The crude product was purified by Flash chromatography to afford compound C171 (15 mg, yield: 14.7%). LCMS: [M+H]⁺=1015, 1017.

### Preparation of 3-(4-(8-(9-(4-((5-bromo-4-((5-(dimethylphosphoryl)quinoxalin-6-yl)amino)pyrimidin-2-yl)amino)-5-methoxy-2-methylphenyl)-3,9-diazaspiro[5.5]undecan-3-yl)oct-1-yn-1-yl)-1-oxoisoindolin-2-yl)piperidine-2,6-dione (C172)

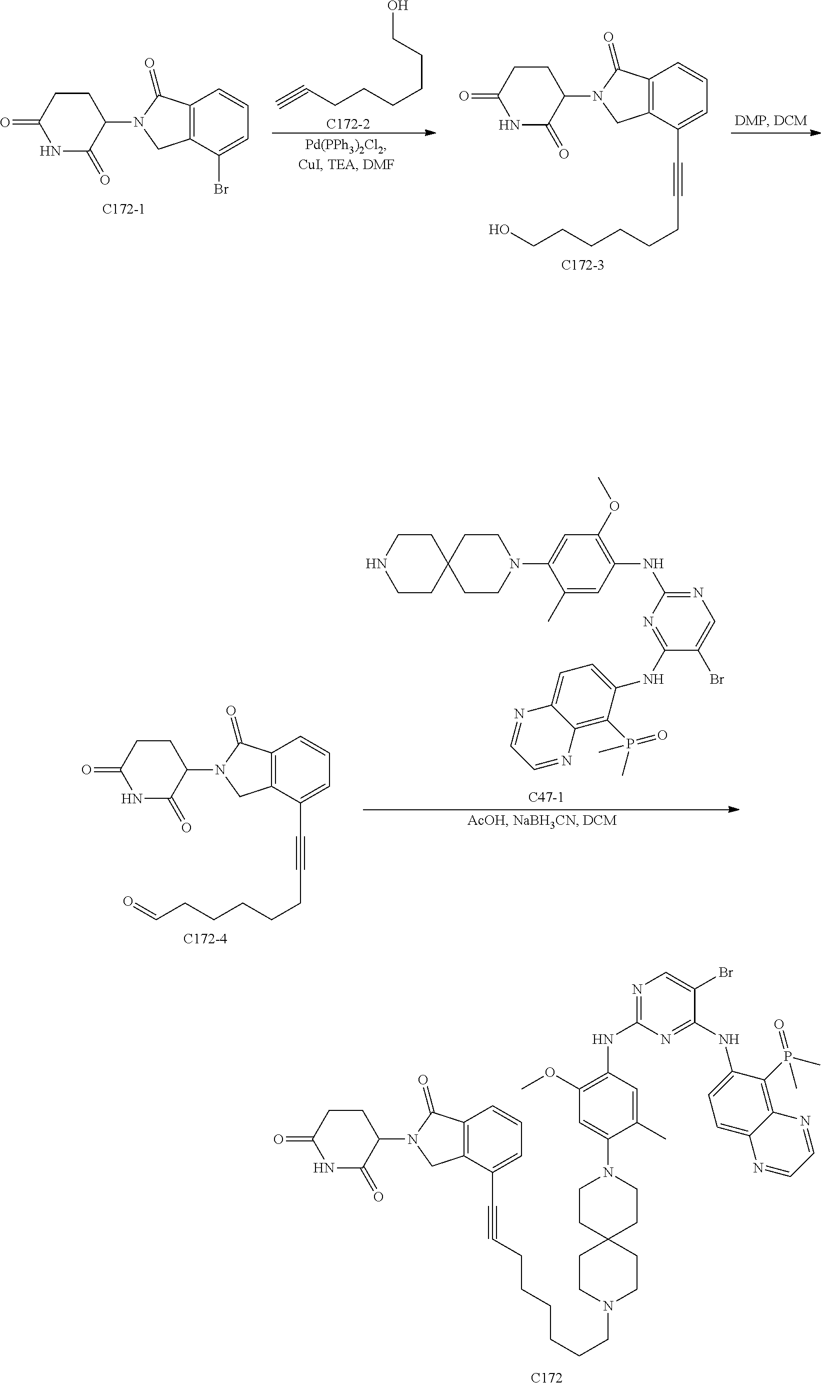

### Step 1

Compound C172-3 (or C1-1) (309 mg, 1 mmol), compound C172-2 (252 mg, 2 mmol), bistriphenylphosphine palladium dichloride (280 mg, 0.4 mmol), cuprous iodide (38.4 mg, 0.2 mmol) and triethylamine (303 mg, 3 mmol) were added to a 100 mL single-necked flask, and then DMIF (20 mL) was added. After the mixture was dissolved, the atmosphere was replaced with nitrogen gas for three times to remove oxygen. The mixture was heated to 70° C. and reacted under nitrogen protection for 5 h. The reaction process was monitored by LCMS. After the reaction was completed, the mixture was cooled and concentrated under reduced pressure to afford a crude product, which was separated by column chromatography to afford 200 mg of light-yellow solid C172-3. Yield: 54.3%. LCMS: [M+H+]⁺=369.4.

### Step 2

Compound C172-3 (200 mg, 0.543 mmol) and dichloromethane (40 mmol) were added to a 100 mL single-necked flask and dissolved with stirring. Dess-Martin reagent (345 mg, 0.814 mmol) was then added. The mixture was heated to 40° C. and reacted for 2 h. The reaction process was monitored by LCMS. The reaction was stopped until the raw materials were basically transformed into the product. The mixture was cooled to 0-5° C. with an ice bath. Saturated sodium bicarbonate solution (10 mL) and sodium thiosulfate solution (10 mL) were successively added and stirred for about 10 minutes. After separation, the aqueous phase was extracted twice with dichloromethane (20 mL for each time). The organic phases were combined, washed with saturated brine (10 mL×2), dried with anhydrous sodium sulfate, and concentrated under reduced pressure to afford 180 mg of white viscous solid C172-4, which was directly used in the next step. LCMS: [M+H]⁺=367.2.

### Step 3

Compound C172-4 (40 mg, 0.11 mmol) and compound C47-1 (59.7 mg, 0.09 mmol) were added to a 25 mL single-necked flask and dissolved in dichloromethane (5 mL). CH₃COOH (10 mg, 0.16 mmol) was then added. The mixture was reacted with stirring at room temperature for 30 minutes. Then sodium cyanobohydride (9.5 mg, 0.15 mmol) was added and reacted at room temperature for 3 h. The reaction process was monitored by LCMS, and the reaction was stopped until the raw materials were basically transformed into the product. Saturated NH₄Cl (5 mL) was added to the mixture and stirred for 5 minutes. The layers were separated, and the aqueous layer was extracted twice with dichloromethane, 10 mL for each time. The organic phases were combined, washed with saturated brine (5 mL×2), dried with anhydrous sodium sulfate, and concentrated under reduced pressure. The residue was separated by Prep-HPLC to afford 20 mg of yellow powder C172. Yield: 18%. LCMS: [M+H]⁺=1001.

### Preparation of 3-(4-(9-(9-(4-((5-bromo-4-((5-(dimethylphosphoryl)quinoxalin-6-yl)amino)pyrimidin-2-yl)amino)-5-methoxy-2-methylphenyl)-3,9-diazaspiro[5.5]undecan-3-yl)non-1-yn-1-yl)-1-oxoisoindolin-2-yl)piperidine-2,6-dione (C173)

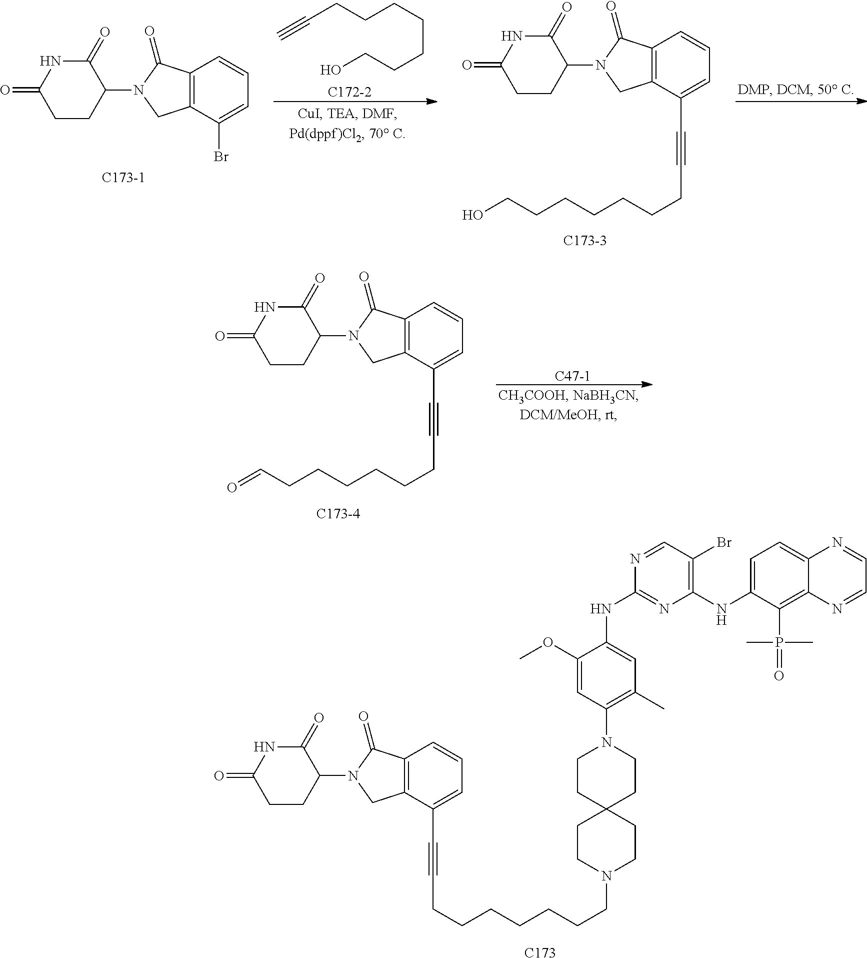

### Step 1

C173-1 (or C1-1) (644 mg, 2.0 mmol), CuI (76 mg, 0.4 mmol) and Pd(dppf)Cl₂ (585 mg, 0.8 mmol) were dispersed in 34 mL of anhydrous DMF. 8-alkynyl-1-nonanol (700 mg, 5.0 mmol) and TEA (606 mg, 6.0 mmol) were added successively under N₂ protection. The mixture was heated to 70° C. and reacted for 20 h. LCMS showed that the reaction was completed. The mixture was cooled to room temperature, diluted with 150 mL of EA, and washed successively with saturated NH₄Cl (2×50 mL), H₂O (2×50 mL) and saturated brine (2×50 mL). The organic layer was dried with anhydrous sodium sulfate, and filtered with suction. The filtrate was concentrated under reduced pressure to afford a crude product, which was purified by RP-Flash chromatography to afford light-yellow solid product C173-2 (210 mg, 27.5%). LCMS: [M+H]⁺=383.

### Step 2

C173-2 (200 mg, 0.524 mmol) was dispersed in 30.0 mL of anhydrous DCM. Dess-Martin reagent (333 mg, 0.785 mmol) was added under N₂ protection. The mixture was heated to 50° C., and reacted under reflux for 2.0 h. TLC showed that the reaction was completed. The mixture was cooled to room temperature, and then 10 mL of saturated NaHCO₃ solution and 10 mL of saturated Na₂S₂O₃ solution were added. The mixture was stirred at room temperature for 5 min. The organic layer was separated, dried with anhydrous sodium sulfate, and filtered with suction. The filtrate was concentrated under reduced pressure to afford a crude product, which was purified by Prep-TLC to afford light-yellow solid product C173-3 (150 mg, 75%). LCMS: [M+H]⁺=381.

### Step 3

C173-3 (45 mg, 0.1184 mmol) and C47-1 (70.8 mg, 0.1066 mmol) were dissolved in a mixture of 8.0 mL of anhydrous DCM and 0.8 mL of anhydrous methanol. CH₃COOH (7.1 mg, 0.1184 mmol, dissolved in 0.5 mL of DCM) was added under nitrogen protection. The mixture was stirred at room temperature for 0.5 h. Then solid NaBH₃CN (11.2 mg, 0.1776 mmol) was added. The mixture was further reacted at room temperature for 2 h. LCMS and TLC showed that the reaction was completed. The mixture was diluted with 20 mL of DCM, and then washed successively with saturated NH₄Cl (1×15 mL) and saturated brine (1×15 mL). The organic layer was dried with anhydrous Na₂SO₄, and filtered with suction. The filtrate was concentrated under reduced pressure to afford a crude product, which was purified by Prep-HPLC to afford 45 mg of yellow solid pure product. Yield: 40.0%. LCMS: [M+H]⁺=1029, 1031.

¹H-NMR: (400 MHz, DMSO-d₆) δ 12.683 (s, 1H), 11.119 (s, 1H), 8.869 (dd, J=2.0 Hz, 9.2 Hz, 3H), 8.281 (s, 1H), 8.263 (s, 1H), 7.721 (d, J=6.8 Hz, 1H), 7.646 (d, J=0.8 Hz, 1H), 7.643 (d, J=6.8 Hz, 1H), 7.539 (d, J=7.6 Hz, 1H), 7.321 (s, 1H), 6.809 (s, 1H), 5.188 (dd, J=5.2 Hz, 13.6 Hz, 1H), 4.477 (d, J=17.6 Hz, 1H), 4.325 (d, J=17.6 Hz, 1H), 3.784 (s, 3H), 2.513-2.495 (m, 10H), 2.494-2.103 (m, 6H), 2.075 (s, 3H), 2.040 (s, 3H), 2.004 (s, 3H), 1.557-1.429 (m, 18H).

### Preparation of 3-(4-(3-(2-(9-(4-((5-bromo-4-((5-(dimethylphosphoryl)quinoxalin-6-yl)amino)pyrimidin-2-yl)amino)-5-methoxy-2-methylphenyl)-3,9-diazaspiro[5.5]undecan-3-yl)ethoxy)prop-1-yn-1-yl)-1-oxoisoindolin-2-yl)piperidine-2,6-dione (C174)

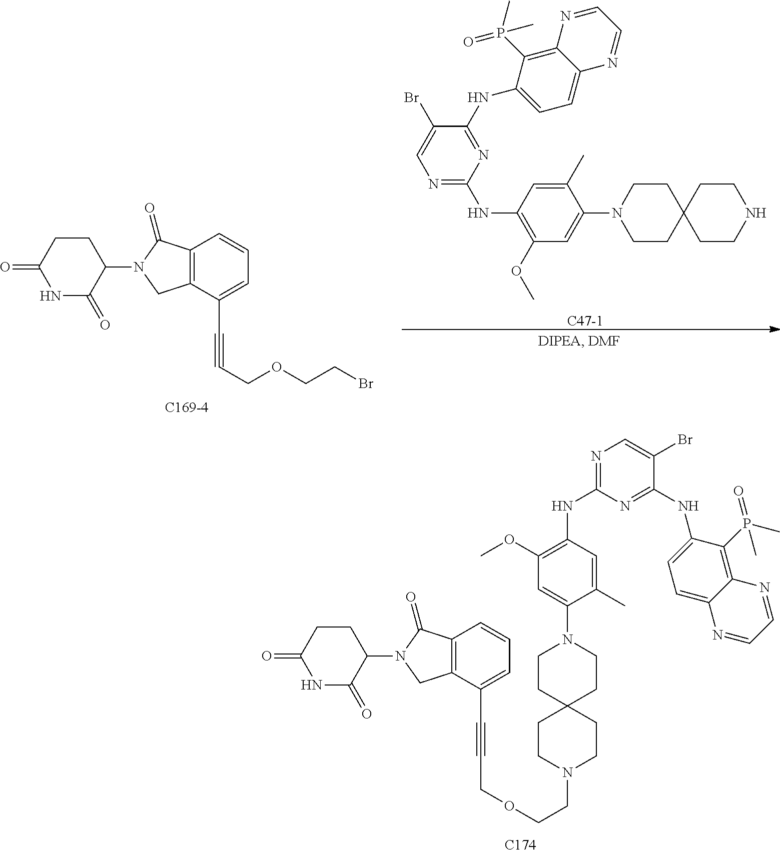 [SMILES: "C174" ; "C169-4" ; "C47-1" ; O=C1CCC(N2Cc3c(C#CCOCCBr)cccc3C2=O)C(=O)N1 ; COc1cc(N2CCC3(CCN(CCOCC#Cc4cccc5c4CN(C4CCC(=O)NC4=O)C5=O)CC3)CC2)c(C)cc1Nc1ncc(Br)c(Nc2ccc3nccnc3c2P(C)(C)=O)n1 ; COc1cc(N2CCC3(CCNCC3)CC2)c(C)cc1Nc1ncc(Br)c(Nc2ccc3nccnc3c2P(C)(C)=O)n1]

Compound C169-4 (2 mg, 0.029 mmol), compound C47-1 (1 mg, 0.029 mmol), diisopropylethylamine (11.2 mg, 0.087 mmol) and DMF (1 mL) were added to a 25 mL single-necked flask equipped with a condenser. The mixture was heated to 70° C. and reacted for 3 h. The reaction process was monitored by LCMS. After the reaction was completed, the mixture was cooled and concentrated under reduced pressure to afford a crude product, which was separated by Prep-HPLC to afford 9 mg of white powder C174. Yield: 31.2%. LCMS: [M+H]⁺=989, 991.

### Preparation of 3-(4-(3-(2-(4-(1-(4-((5-chloro-4-((2-(dimethylphosphoryl)phenyl)amino)pyrimidin-2-yl)amino)-3-methoxyphenyl)piperidin-4-yl)piperazin-1-yl)ethoxy)prop-1-yn-1-yl)-1-oxoisoindolin-2-yl)piperidine-2,6-dione (C176)

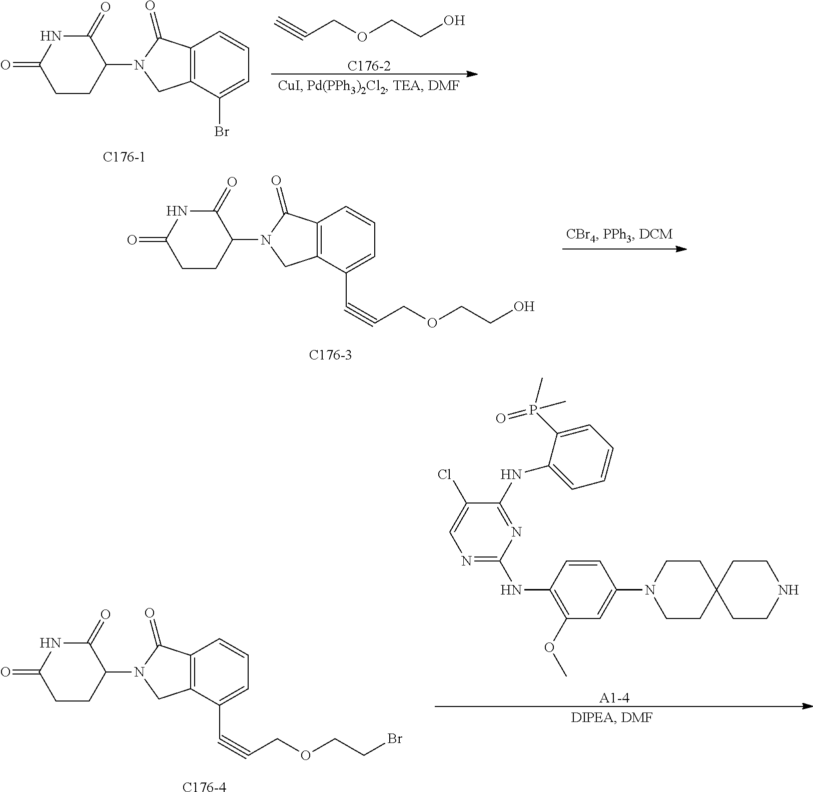

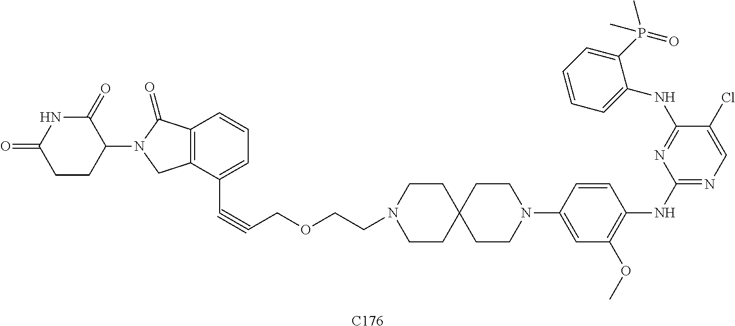 [SMILES: COc1cc(N2CCC3(CCN(CCOCC#Cc4cccc5c4CN(C4CCC(=O)NC4=O)C5=O)CC3)CC2)ccc1Nc1ncc(Cl)c(Nc2ccccc2P(C)(C)=O)n1 ; "C176"]

### Step 1

Compound C176-1 (or C1-1) (322 mg, 1.00 mmol), compound C176-2 (200 mg, 2.00 mmol), cuprous iodide (38 mg, 0.20 mmol), Pd(PPh₃)₂Cl₂ (280 mg, 0.40 mmol) and triethylamine (303 mg, 3.00 mmol) were dissolved in dry DMF (10 mL). The mixture was stirred at 70° C. under nitrogen protection overnight. The mixture was cooled to room temperature. The solvent was removed under reduced pressure, and the crude product was separated by column chromatography to afford compound C176-3 (80 mg, yield: 23.4%). LCMS: [M+H]⁺=343.

### Step 2

Compound C176-3 (52 mg, 0.152 mmol) and carbon tetrabromide (101 mg, 0.305 mmol) were dissolved in dichloromethane (20 mL). The mixture was stirred at room temperature for 2 h. The mixture was cooled to 0° C. A solution of PPh₃ (80 mg, 0.305 mmol) in dichloromethane (2 mL) was added dropwise. The mixture was naturally warmed to room temperature, and then stirred at 55° C. for 2 h. The solvent was removed under reduced pressure. The crude product was separated by column chromatography to afford compound C176-4 (20 mg, yield: 32.8%). LCMS: [M+H]⁺=405, 407.

### Step 3

Compound C176-4 (20 mg, 0.050 mmol), compound A1-4 (31 mg, 0.054 mmol), and DIEA (32 mg, 0.248 mmol) were dissolved in 1 mL of DMF. The mixture was stirred at 80° C. under nitrogen protection for 5 h. LCMS showed that the reaction was completed. The solvent was removed at 40° C. under reduced pressure, and the crude product was purified by Flash chromatography to afford C176 (18 mg, yield: 40.9%). LCMS: [M+H]⁺=894. ¹H NMR (400 MHz, DMSO-d₆) δ 11.17 (s, 1H), 11.01 (s, 1H), 8.48 (s, 1H), 8.05 (d, 2H), 7.75 (dd, 2H), 7.54 (dt, 2H), 7.36 (dd, 2H), 7.09 (t, 1H), 6.62 (d, 1H), 6.47 (dd, 1H), 5.75 (s, 1H), 5.16 (dd, 1H), 4.50 (d, 1H), 4.35 (d, 1H), 3.76 (s, 3H), 3.71 (d, 2H), 3.65 (t, 2H), 3.28 (s, 1H), 3.00-2.87 (m, 2H), 2.66 (d, 4H), 2.57 (s, 1H), 2.44 (d, 4H), 2.29 (s, 2H), 2.01 (p, 3H), 1.84 (d, 2H), 1.76 (d, 6H), 1.57-1.42 (m, 3H).

### Preparation of 3-(4-(8-(4-(1-(4-((5-chloro-4-((2-(dimethylphosphoryl)phenyl)amino)pyrimidin-2-yl)amino)-3-methoxyphenyl)piperidin-4-yl)piperazin-1-yl)oct-1-yn-1-yl)-1-oxoisoindolin-2-yl)piperidine-2,6-dione (C177)

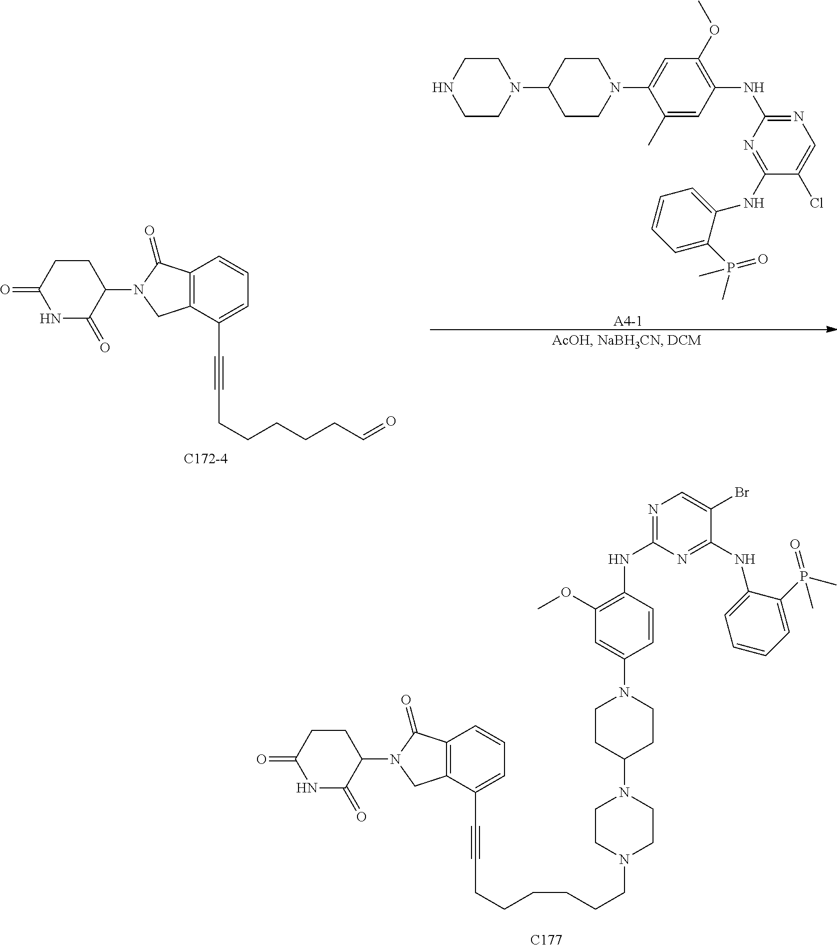

Compound C172-4 (40 mg, 0.11 mmol) and A1-4 (51.2 mg, 0.09 mmol) were added to a 25 mL single-necked flask and dissolved in dichloromethane (5 mL). CH₃COOH (10 mg, 0.16 mmol) was then added. The mixture was reacted with stirring at room temperature for 30 minutes. Then sodium cyanobohydride (9.5 mg, 0.15 mmol) was added and reacted at room temperature for 3 h. The reaction process was monitored by LCMS. After the reaction was completed, saturated NH₄Cl (5 mL) was added to the mixture and stirred for 5 minutes. The layers were separated, and the aqueous layer was extracted with dichloromethane (10 mL×2). The organic phases were combined, washed with saturated brine (5 mL×2), dried with anhydrous sodium sulfate, and concentrated under reduced pressure. The residue was separated by Prep-HPLC to afford 18 mg of yellow powder. Yield: 19.5%. LCMS: [M+H]⁺=920.

### Preparation of 3-(4-(9-(4-(1-(4-((5-chloro-4-((2-(dimethylphosphoryl)phenyl)amino)pyrimidin-2-yl)amino)-3-methoxyphenyl)piperidin-4-yl)piperazin-1-yl)non-1-yn-1-yl)-1-oxoisoindolin-2-yl)piperidine-2,6-dione (C178)

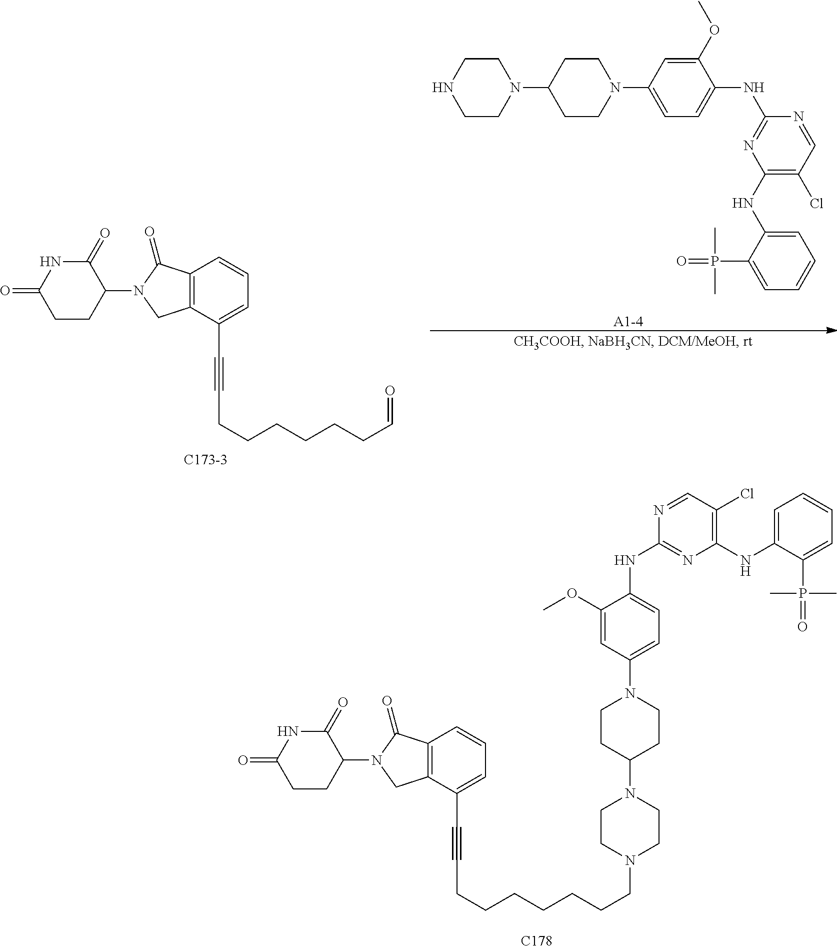 [SMILES: "C178" ; "C173-3" ; O=CCCCCCCC#Cc1cccc2c1CN(C1CCC(=O)NC1=O)C2=O ; COc1cc(N2CCC(N3CCN(CCCCCCCC#Cc4cccc5c4CN(C4CCC(=O)NC4=O)C5=O)CC3)CC2)ccc1Nc1ncc(Cl)c(Nc2ccccc2P(C)(C)=O)n1 ; COc1cc(N2CCC(N3CCNCC3)CC2)ccc1Nc1ncc(Cl)c(Nc2ccccc2P(C)(C)=O)n1 ; "A1-4"]

C173-3 (48 mg, 0.1263 mmol) and A1-4 (64.7 mg, 0.1137 mmol) were dissolved in a mixture of 8.0 mL of anhydrous DCM and 0.8 mL of anhydrous methanol. CH₃COOH (7.6 mg, 0.1263 mmol, dissolved in 0.5 mL of DCM) was added under nitrogen protection. The mixture was stirred at room temperature for 0.5 h. Then solid NaBH₃CN (11.9 mg, 0.1895 mmol) was added. The mixture was further reacted at room temperature for 2 h. LCMS and TLC showed that the reaction was completed. The mixture was diluted with 20 mL of DCM, and then washed successively with saturated NH₄Cl (1×15 mL) and saturated brine (1×15 mL). The organic layer was dried with anhydrous Na₂SO₄, and filtered with suction. The filtrate was concentrated under reduced pressure to afford a crude product. The crude product was purified by Prep-TLC to afford 55 mg of crude product, which was further purified by Prep-HPLC to afford 35 mg of white solid pure product. Yield: 29.7%. LCMS: [M+H]⁺=934. ¹H-NMR: (400 MHz, DMSO-d₆) δ 11.190 (s, 1H), 11.089 (s, 1H), 8.481 (brs, 1H), 8.092 (d, J=10.0 Hz, 2H), 7.721 (d, J=7.6 Hz, 1H), 7.644 (d, J=7.6 Hz, 1H), 7.539-7.502 (m, 2H), 7.340 (dd, J=8.4 Hz, 17.2 Hz, 2H), 7.070 (t, J=14.4 Hz, 1H), 6.621 (s, 1H), 6.476 (d, J=8.8 Hz, 1H), 5.184 (dd, J=4.8 Hz, 13.2 Hz, 1H), 4.474 (d, J=17.6 Hz, 1H), 4.321 (d, J=17.6 Hz, 1H), 3.757-3.694 (m, 5H), 2.932-2.842 (m, 2H), 2.636-2.530 (m, 3H), 2.444-2.380 (m, 4H), 2.357-2.160 (m, 7H), 1.989-1.933 (m, 2H), 1.809-780 (m, 2H), 1.743 (s, 3H), 1.709 (s, 3H), 1.566-1.355 (m, 8H), 1.319-1.157 (m, 5H).

### Preparation of 3-(4-(10-(4-(1-(4-((5-chloro-4-((2-(dimethylphosphoryl)phenyl)amino)pyrimidin-2-yl)amino)-3-methoxyphenyl)piperidin-4-yl)piperazin-1-yl)dec-1-yn-1-yl)-1-oxoisoindolin-2-yl)piperidine-2,6-dione (C179)

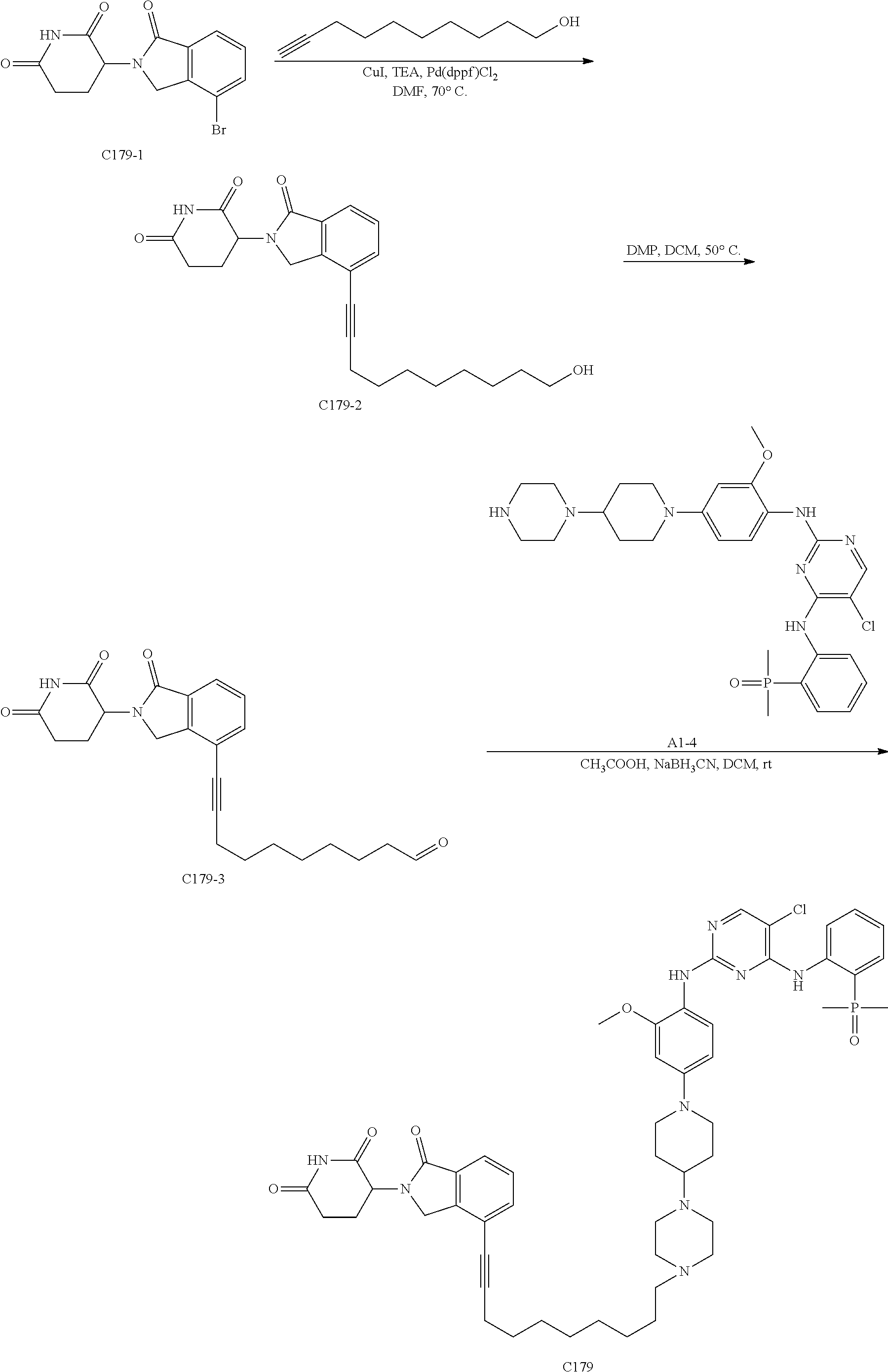

### Step 1

C179-1 (or C1-1) (322 mg, 1.0 mmol), CuI (38 mg, 0.2 mmol) and Pd(dppf)Cl₂ (292.4 mg, 0.4 mmol) were dispersed in 15 mL of anhydrous DMF. 9-alkynyl-1-decanol (385 mg, 2.5 mmol) and TEA (303 mg, 3.0 mmol) were added successively under N₂ protection. The mixture was heated to 70° C. and reacted for 20 h. LCMS showed that the reaction was completed. The mixture was cooled to room temperature, diluted with 80 mL of EA, and then washed successively with saturated NH₄Cl (2×50 mL), H₂O (2×50 mL) and saturated brine (2×50 mL). The organic layer was dried with anhydrous sodium sulfate, and filtered with suction. The filtrate was concentrated under reduced pressure to afford a crude product, which was purified by RP-Flash chromatography to afford light-yellow solid product C179-2 (105 mg, 26.5%). LCMS: [M+H]⁺=397.

### Step 2

C179-2 (35 mg, 0.088 mmol) was dissolved in 10.0 mL of anhydrous DCM. Dess-Martin reagent (75 mg, 0.176 mmol) was added under N₂ protection. The mixture was heated to 50° C., and reacted under reflux for 2.0 h. TLC showed that the reaction was completed. The mixture was cooled to room temperature, and then 10 mL of saturated NaHCO₃ solution and 10 mL of saturated Na₂S₂O₃ solution were added. The mixture was stirred vigorously at room temperature for 5 min. The organic layer was separated, dried with anhydrous sodium sulfate, and filtered with suction. The filtrate was concentrated under reduced pressure to afford a crude product, which was purified by Prep-TLC to afford light-yellow solid product C179-3 (15 mg, 42.8%). LCMS: [M+H]⁺=395.

### Step 3

C179-3 (15 mg, 0.0381 mmol) and A1-4 (19.5 mg, 0.0343 mmol) were dissolved in 3.0 mL of anhydrous DCM. CH₃COOH (2.3 mg, 0.0381 mmol, dissolved in 0.5 mL of DCM) was added under nitrogen protection. The mixture was stirred at room temperature for 0.5 h. Then solid NaBH₃CN (3.59 mg, 0.0572 mmol) was added. The mixture was further reacted at room temperature for 2 h. LCMS and TLC showed that the reaction was completed. The mixture was directly purified by Prep-TLC to afford 15 mg of crude product, which was further purified by Prep-HPLC to afford white solid pure product C179 (7.0 mg, yield: 19.5%). LCMS: [M+H]⁺=948.

### Preparation of 3-(4-(3-(3-(4-(1-(4-((5-chloro-4-((2-(dimethylphosphoryl)phenyl)amino)pyrimidin-2-yl)amino)-3-methoxyphenyl)piperidin-4-yl)piperazin-1-yl)propoxy)prop-1-yn-1-yl)-1-oxoisoindolin-2-yl)piperidine-2,6-dione (C181)

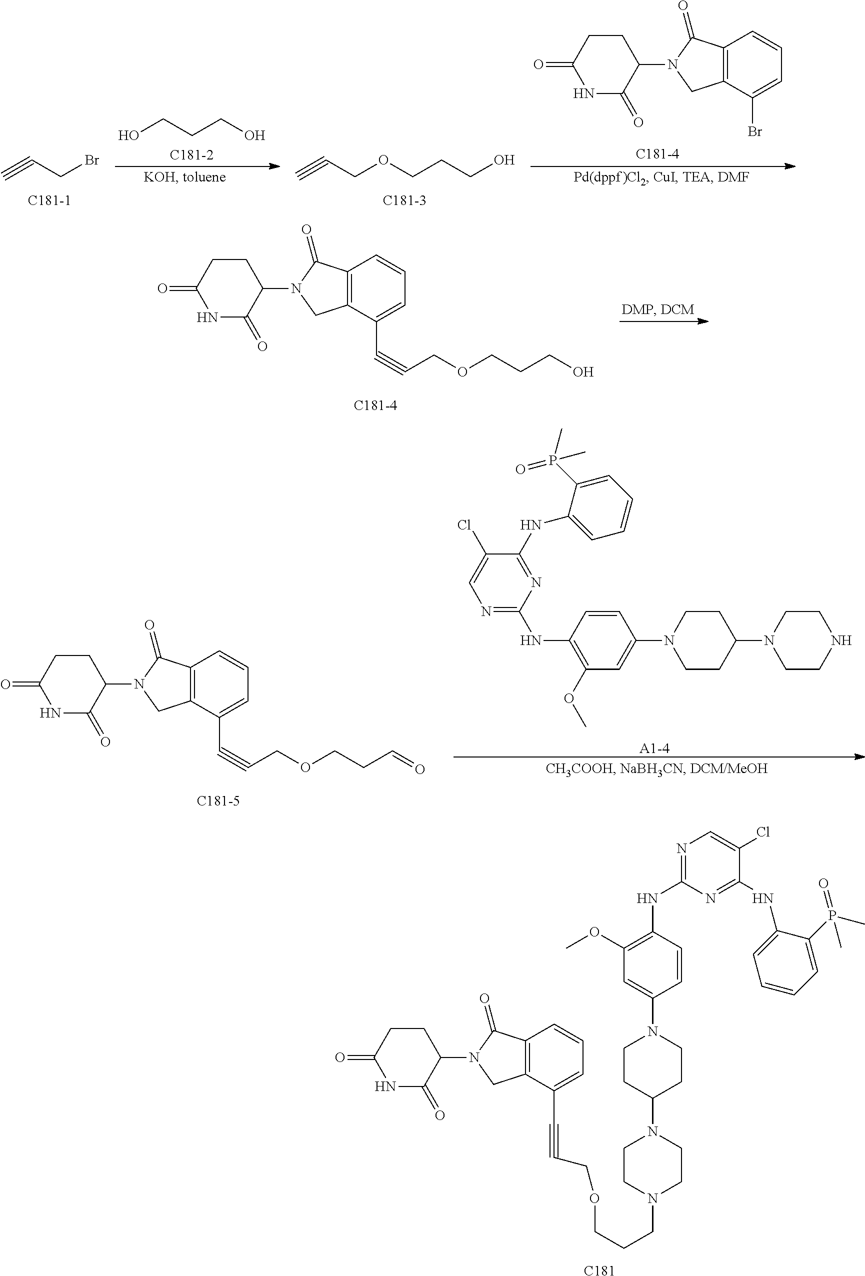

### Step 1

At 0° C., potassium hydroxide (3.75 g, 66.8 mmol) was added portionwise to a solution of a mixture of compound C181-1 (3.68 g, 25.0 mmol) and compound C181-2 (1.99 g, 26.2 mmol) in toluene. The mixture was stirred vigorously, then warmed to room temperature, and further stirred for 1 h. The mixture was diluted with water, and extracted with 100 mL of ethyl acetate. The organic phase was dried with anhydrous sodium sulfate, and filtered. The solvent was removed under reduced pressure, and the crude product was separated by column chromatography to afford 1.2 g of liquid. Yield: 42.9%.

### Step 2

Compound C181-4 (or C1-1) (200 mg, 0.62 mmol), compound C181-3 (566 mg, 4.97 mmol), cuprous iodide (24 mg, 0.12 mmol), Pd(dppf)Cl₂ (174 mg, 0.25 mmol) and triethylamine (188 mg, 1.86 mmol) were dissolved in 5 mL of anhydrous DMF. The mixture was reacted with stirring at 70° C. under nitrogen protection for 5 h. The solvent was removed under reduced pressure, and the crude product was purified by thin layer chromatography to afford off-white solid compound C181-4 (60 mg, yield: 27.1%). LCMS: [M+H]⁺=357.

### Step 3

Compound C181-4 (20 mg, 0.056 mmol) and Dess-Martin reagent (36 mg, 0.084 mmol) were dissolved in 20 mL of dichloromethane and stirred at 50° C. for 2 h. The mixture was cooled to room temperature, and quenched with saturated sodium bicarbonate aqueous solution. The organic phase was separated, and the aqueous phase was extracted with dichloromethane for three times. The organic phases were combined, dried with anhydrous sodium sulfate, and filtered. The filtrate was concentrated, and the crude product was purified by TLC to afford a white solid compound C181-5 (18 mg, yield: 90.0%). LCMS: [M+H]⁺=355.

### Step 4

Compound C181-5 (18 mg, 0.051 mmol) and compound A1-4 (32 mg, 0.056 mmol) were dissolved in 3 mL of dichloromethane/methanol (2/1, v/v), and 1 drop of glacial acetic acid was added dropwise. The mixture was stirred at room temperature under nitrogen protection for 1 h. Then sodium cyanobohydride (3.2 mg, 0.051 mmol) was added. The mixture was further stirred for 1 h. The solvent was removed under reduced pressure below 40° C. The crude product was purified by Flash chromatography to afford off-white solid compound C181 (15 mg, yield: 32.6%). LCMS: [M+H]⁺=908. ¹H NMR (400 MHz, DMSO-d₆) δ 11.15 (s, 1H), 11.00 (s, 1H), 8.50 (dr, 1H), 8.04 (d, 2H), 7.71 (dd, 1H), 7.63 (d, 1H), 7.57-7.44 (m, 2H), 7.36 (m, 2H), 7.06 (d, 1H), 6.64 (s, 1H), 6.43 (d, 1H), 5.29 (t, 1H), 5.13 (dd, 1H), 4.46 (d, 1H), 4.38 (s, 2H), 4.31 (d, 1H), 3.72 (s, 3H), 3.67 (d, 2H), 3.53 (t, 2H), 2.98 (m, 1H), 2.67 (m, 4H), 2.59 (m, 3H), 2.25 (m, 4H), 2.02 (m, 2H), 1.96 (q, 2H), 1.69 (s, 3H), 1.68 (s, 3H), 1.45 (s, 2H), 1.21 (d, 4H).

### Preparation of 3-(4-(3-(4-(4-(1-(4-((5-chloro-4-((2-(dimethylphosphoryl)phenyl)amino)pyrimidin-2-yl)amino)-3-methoxyphenyl)piperidin-4-yl)piperazin-1-yl)butoxy)prop-1-yn-1-yl)-1-oxoisoindolin-2-yl)piperidine-2,6-dione (C182)

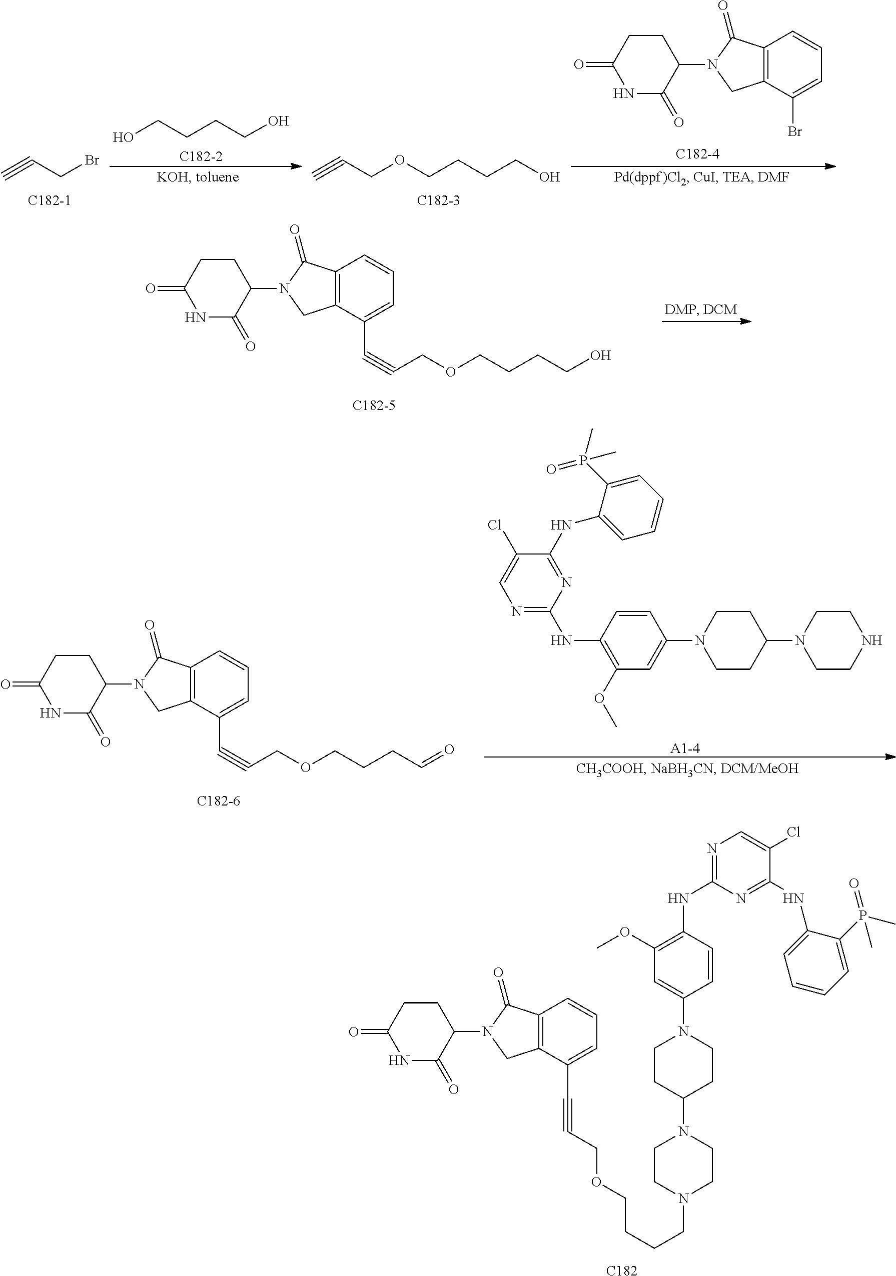

### Step 1

At 0° C., potassium hydroxide (3.75 g, 66.8 mmol) was added portionwise to a solution of a mixture of compound C182-1 (3.68 g, 25.0 mmol) and compound C182-2 (2.36 g, 26.2 mmol) in toluene. The mixture was stirred vigorously, then warmed to room temperature, and further stirred for 1 h. The mixture was diluted with water, and extracted with 100 mL of ethyl acetate. The organic phase was dried with anhydrous sodium sulfate, and filtered. The solvent was removed under reduced pressure, and the crude product was separated by column chromatography to afford 1.4 g of liquid C182-3. Yield: 45.2%.

### Step 2

Compound C182-4 (or C1-1) (200 mg, 0.62 mmol), compound C182-3 (6366 mg, 4.97 mmol), cuprous iodide (24 mg, 0.12 mmol), Pd(dppf)Cl₂ (174 mg, 0.25 mmol) and triethylamine (188 mg, 1.86 mmol) were dissolved in 5 mL of anhydrous DMF. The mixture was reacted with stirring at 70° C. under nitrogen protection for 5 h. The solvent was removed under reduced pressure, and the crude product was purified by thin layer chromatography to afford off-white solid compound C182-5 (60 mg, yield: 26.1%). LCMS: [M+H]⁺=371.

### Step 3

Compound C182-5 (30 mg, 0.081 mmol) and Dess-Martin reagent (52 mg, 0.1224 mmol) were dissolved in 20 mL of dichloromethane and stirred at 50° C. for 2 h. The mixture was cooled to room temperature, and quenched with saturated sodium bicarbonate aqueous solution. The organic phase was separated, and the aqueous phase was extracted with dichloromethane for three times. The organic phases were combined, dried with anhydrous sodium sulfate, and filtered. The filtrate was concentrated, and the crude product was purified by TLC to afford a white solid compound C182-6 (28 mg, yield: 93.3%). LCMS: [M+H]⁺=369.

### Step 4

Compound C182-6 (20 mg, 0.051 mmol) and compound A1-4 (32 mg, 0.056 mmol) were dissolved in 3 mL of dichloromethane/methanol (2/1), and 1 drop of glacial acetic acid was added dropwise. The mixture was stirred at room temperature under nitrogen protection for 1 h. Then sodium cyanobohydride (3.2 mg, 0.051 mmol) was added. The mixture was further stirred for 1 h. The solvent was removed under reduced pressure below 40° C. The crude product was purified by Flash chromatography to afford off-white solid compound C182 (10 mg, yield: 20.8%). LCMS: [M+H]⁺=948. ¹H NMR (400 MHz, DMSO-d₆) δ 11.18 (s, 1H), 11.05 (s, 1H), 8.50 (dr, 1H), 8.08 (d, 2H), 7.78 (dd, 1H), 7.72 (d, 1H), 7.56-7.44 (m, 2H), 7.36 (m, 2H), 7.09 (d, 1H), 6.62 (s, 1H), 6.48 (d, 1H), 5.32 (t, 1H), 5.15 (dd, 1H), 4.53 (d, 1H), 4.37 (s, 2H), 4.32 (d, 1H), 3.75 (s, 3H), 3.69 (d, 2H), 3.54 (t, 2H), 2.97 (m, 1H), 2.66 (m, 4H), 2.56 (m, 3H), 2.49 (m, 4H), 2.02 (m, 2H), 1.96 (q, 2H), 1.69 (s, 3H), 1.68 (s, 3H), 1.45 (s, 3H), 1.23 (d, 5H).

### Preparation of 3-(4-(9-(4-(1-(4-((5-bromo-4-((5-(dimethylphosphoryl)quinoxalin-6-yl)amino)pyrimidin-2-yl)amino)-5-methoxy-2-methylphenyl)piperidin-4-yl)piperazin-1-yl)non-1-yn-1-yl)-1-oxoisoindolin-2-yl)piperidine-2,6-dione (C183)

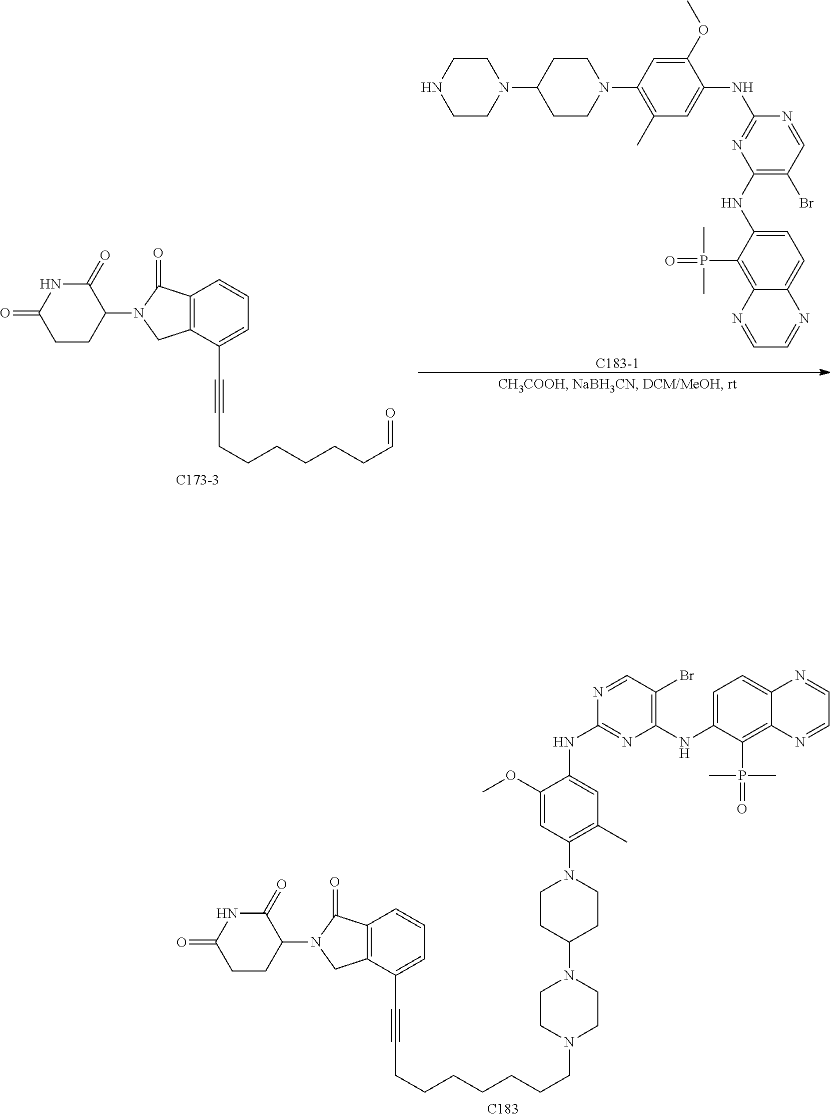 [SMILES: "C183" ; "C183-1" ; "C173-3" ; O=CCCCCCCC#Cc1cccc2c1CN(C1CCC(=O)NC1=O)C2=O ; COc1cc(N2CCC(N3CCN(CCCCCCCC#Cc4cccc5c4CN(C4CCC(=O)NC4=O)C5=O)CC3)CC2)c(C)cc1Nc1ncc(Br)c(Nc2ccc3nccnc3c2P(C)(C)=O)n1 ; COc1cc(N2CCC(N3CCNCC3)CC2)c(C)cc1Nc1ncc(Br)c(Nc2ccc3nccnc3c2P(C)(C)=O)n1]

C173-3 (30 mg, 0.079 mmol) and C183-1 (or C158-1) (48.2 mg, 0.071 mmol) were dissolved in a mixture of 4.0 mL of anhydrous DCM and 0.4 mL of anhydrous methanol. CH₃COOH (4.74 mg, 0.079 mmol, dissolved in 0.5 mL of DCM) was added under nitrogen protection. The mixture was stirred at room temperature for 0.5 h. Then solid NaBH₃CN (7.44 mg, 0.1185 mmol) was added. The mixture was further reacted at room temperature for 2 h. LCMS and TLC showed that the reaction was completed. The mixture was diluted with 20 mL of DCM, and then washed successively with saturated NH₄Cl (1×15 mL) and saturated brine (1×15 mL). The organic layer was dried with anhydrous Na₂SO₄, and filtered with suction. The filtrate was concentrated under reduced pressure to afford a crude product, which was purified by Prep-HPLC to afford 25 mg of yellow solid pure product. Yield: 30.4%. LCMS: [M+H]⁺=1044, 1046. ¹H-NMR: (400 MHz, DMSO-d₆) δ 12.696 (s, 1H), 11.089 (s, 1H), 8.876 (dd, J=2.0 Hz, 7.6 Hz, 3H), 8.272 (d, J=1.6 Hz, 2H), 7.722 (d, J=7.6 Hz, 1H), 7.646 (d, J=6.8 Hz, 1H), 7.629 (t, J=0.8 Hz, 1H), 7.347 (s, 1H), 6.748 (s, 1H), 5.187 (dd, J=5.2 Hz, 13.2 Hz, 1H), 4.478 (d, J=17.6 Hz, 1H), 4.325 (d, J=17.6 Hz, 1H), 3.775 (s, 3H), 3.345-2.831 (m, 4H), 2.522-2.469 (m, 4H), 2.468-2.185 (m, 9H), 2.087 (s, 3H), 2.043 (s, 3H), 2.007 (s, 3H), 1.574-1.560 (m, 2H), 1.432-1.230 (m, 16H).

### Preparation of 3-(4-(3-(2-(4-(1-(4-((5-chloro-4-((2-(dimethylphosphoryl)phenyl)amino)pyrimidin-2-yl)amino)-3-methoxyphenyl)piperidin-4-yl)piperazin-1-yl)ethoxy)prop-1-yn-1-yl)-6-fluoro-1-oxoisoindolin-2-yl)piperidine-2,6-dione (C184)

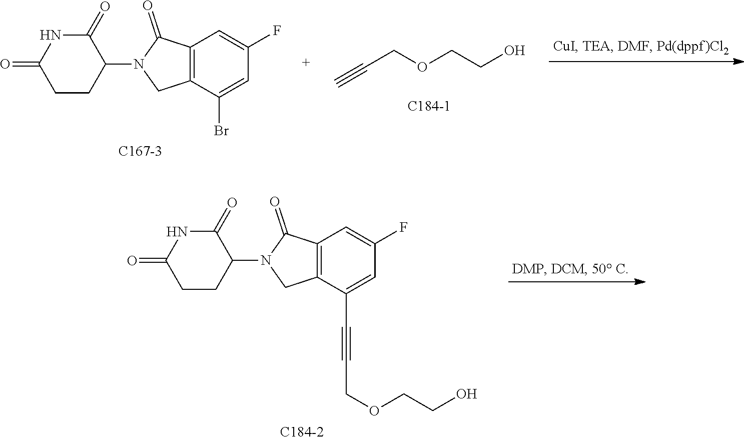

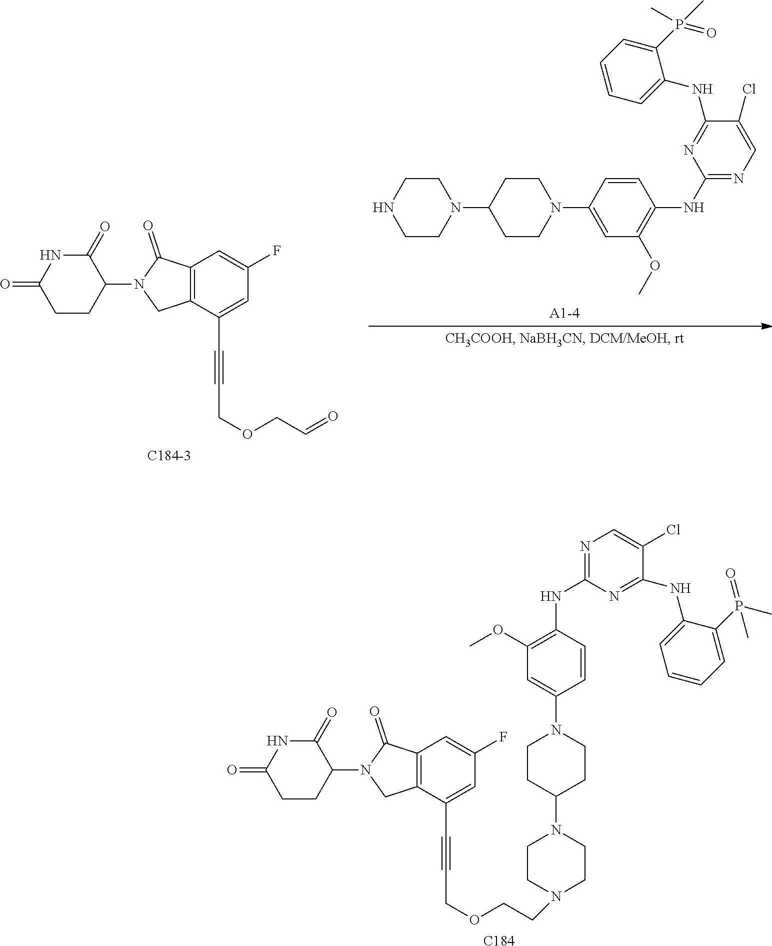 [SMILES: "C184" ; "C184-3" ; O=CCOCC#Cc1cc(F)cc2c1CN(C1CCC(=O)NC1=O)C2=O ; COc1cc(N2CCC(N3CCN(CCOCC#Cc4cc(F)cc5c4CN(C4CCC(=O)NC4=O)C5=O)CC3)CC2)ccc1Nc1ncc(Cl)c(Nc2ccccc2P(C)(C)=O)n1 ; COc1cc(N2CCC(N3CCNCC3)CC2)ccc1Nc1ncc(Cl)c(Nc2ccccc2P(C)(C)=O)n1]

### Step 1

C167-3 (340 mg, 1.0 mmol), CuI (19.0 mg, 0.10 mmol) and Pd(dppf)Cl₂ (73.1 mg, 0.10 mmol) were dispersed in 17.0 mL of anhydrous DMF. C184-1 (200.2 mg, 2.0 mmol) and TEA (303 mg, 3.0 mmol) were added successively under N₂ protection. The mixture was heated to 70° C. and reacted for 20 h. LCMS showed that the reaction was completed. The mixture was cooled to room temperature, diluted with 100 mL of ethyl acetate, and then washed successively with saturated NH₄Cl (2×50 mL), H₂O (2×50 mL) and saturated brine (2×50 mL). The organic layer was dried with anhydrous sodium sulfate, and filtered with suction. The filtrate was concentrated under reduced pressure to afford a crude product, which was purified by RP-Flash chromatography to afford white solid product C184-2 (175 mg, 48.6%). LCMS: [M+H]⁺=361.

### Step 2

C184-2 (70 mg, 0.195 mmol) was dissolved in 14.0 mL of anhydrous DCM, and Dess-Martin reagent (124 mg, 0.293 mmol) was added under N₂ protection. The mixture was heated to 50° C., and reacted under reflux for 2.0 h. TLC showed that the reaction was completed. The mixture was cooled to room temperature, and then 10 mL of saturated NaHCO₃ solution and 10 mL of saturated Na₂S₂O₃ solution were added. The mixture was stirred vigorously at room temperature for 5 min. The organic layer was separated, dried with anhydrous sodium sulfate, and filtered with suction. The filtrate was concentrated under reduced pressure to afford a crude product. The crude product was purified by Prep-TLC to afford light-yellow solid product C184-3 (40 mg, 57.2%). LCMS: [M+H]⁺=359.

### Step 3

C184-3 (40 mg, 0.112 mmol) and A1-4 (51 mg, 0.0896 mmol) were dissolved in a mixture of 5.0 mL of anhydrous DCM and 0.5 mL of anhydrous methanol. CH₃COOH (10.08 mg, 0.168 mmol) was added under nitrogen protection. The mixture was stirred at room temperature for 0.5 h. Then solid NaBH₃CN (14.1 mg, 0.224 mmol) was added. The mixture was further reacted at room temperature for 2 h. LCMS and TLC showed that the reaction was completed. The mixture was purified by Prep-HPLC to afford white solid pure product C184 (20 mg, yield: 19.6%). LCMS: [M+H]⁺=912.

### Preparation of 3-(4-(7-(9-(4-((5-bromo-4-((5-(dimethylphosphoryl)quinoxalin-6-yl)amino)pyrimidin-2-yl)amino)-5-methoxy-2-methylphenyl)-3,9-diazaspiro[5.5]undecan-3-yl)hept-1-yn-1-yl)-6-fluoro-1-oxoisoindolin-2-yl)piperidine-2,6-dione (C185)

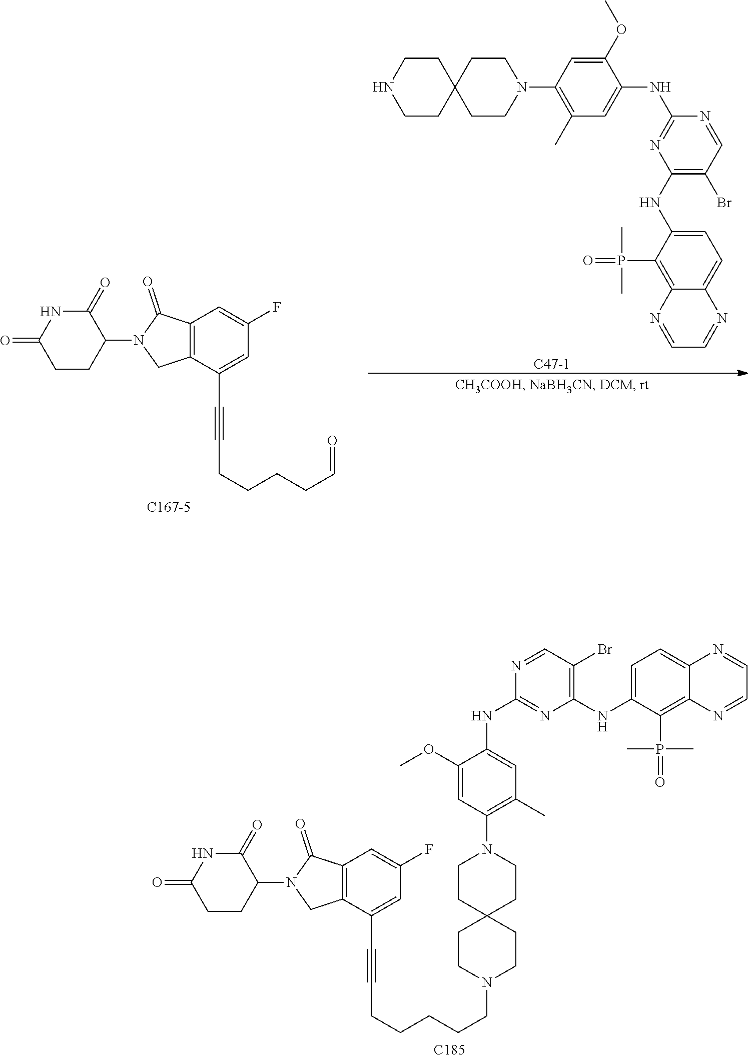 [SMILES: "C185" ; "C167-5" ; "C47-1" ; O=CCCCCC#Cc1cc(F)cc2c1CN(C1CCC(=O)NC1=O)C2=O ; COc1cc(N2CCC3(CCN(CCCCCC#Cc4cc(F)cc5c4CN(C4CCC(=O)NC4=O)C5=O)CC3)CC2)c(C)cc1Nc1ncc(Br)c(Nc2ccc3nccnc3c2P(C)(C)=O)n1 ; COc1cc(N2CCC3(CCNCC3)CC2)c(C)cc1Nc1ncc(Br)c(Nc2ccc3nccnc3c2P(C)(C)=O)n1]

### Step 1

C167-5 (18.0 mg, 0.0486 mmol) and C47-1 (32.3 mg, 0.0486 mmol) were dissolved in 4.0 mL of anhydrous DCM. CH₃COOH (2.9 mg, 0.0486 mmol, dissolved in 0.5 mL of DCM) was added under nitrogen protection. The mixture was stirred at room temperature for 0.5 h. Then solid NaBH₃CN (6.1 mg, 0.0972 mmol) was added. The mixture was further reacted at room temperature for 2 h. LCMS and TLC showed that the reaction was completed. The mixture was diluted with 20 mL of DCM, and washed successively with saturated NH₄Cl (1×15 mL) and saturated brine (1×15 mL). The organic layer was dried with anhydrous Na₂SO₄, and filtered with suction. The filtrate was concentrated under reduced pressure to afford a crude product, which was purified by Prep-HPLC to afford 13 mg of yellow solid pure product. Yield: 26.3%. LCMS: [M+H]⁺=1019, 1021. ¹H-NMR: (400 MHz, DMSO-d₆) δ 11.688 (s, 1H), 11.051 (s, 1H), 8.877 (dd, J=2.0 Hz, 11.2 Hz, 3H), 8.307 (s, 1H), 8.272 (d, J=2.0 Hz, 1H), 7.936 (d, J=7.6 Hz, 1H), 7.586-7.541 (m, 2H), 7.328 (s, 1H), 6.820 (s, 1H), 5.191 (dd, J=5.2 Hz, 13.2 Hz, 1H), 4.474 (dd, J=18.8 Hz, 60.0 Hz, 2H), 3.787 (s, 3H), 3.505 (s, 1H), 3.347 (s, 3H), 2.942-2.619 (m, 6H), 2.570 (s, 4H), 2.081-2.004 (m, 9H), 1.639-1.432 (m, 11H), 1.232 (s, 5H).

### Preparation of 3-(4-(6-(4-(1-(4-((5-bromo-4-((5-(dimethylphosphoryl)quinoxalin-6-yl)amino)pyrimidin-2-yl)amino)-5-methoxy-2-methylphenyl)piperidin-4-yl)piperazin-1-yl)hex-1-yn-1-yl)-1-oxoisoindolin-2-yl)piperidine-2,6-dione (C186)

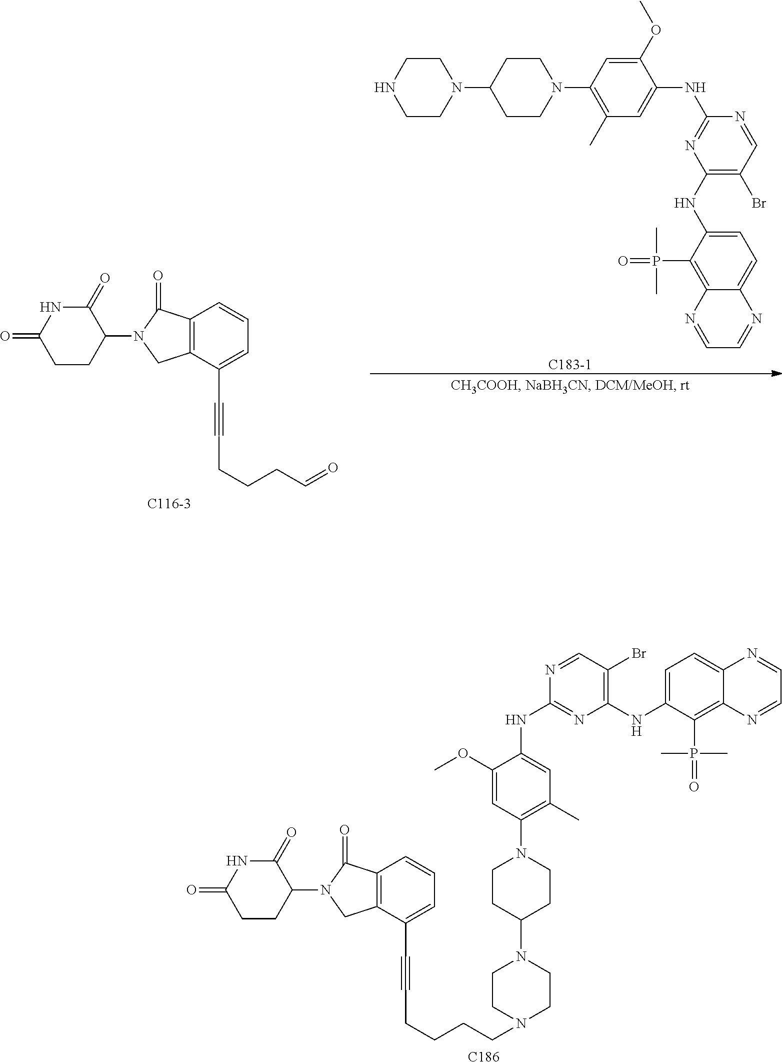 [SMILES: "C186" ; "C183-1" ; "C116-3" ; O=CCCCC#Cc1cccc2c1CN(C1CCC(=O)NC1=O)C2=O ; COc1cc(N2CCC(N3CCN(CCCCC#Cc4cccc5c4CN(C4CCC(=O)NC4=O)C5=O)CC3)CC2)c(C)cc1Nc1ncc(Br)c(Nc2ccc3nccnc3c2P(C)(C)=O)n1 ; COc1cc(N2CCC(N3CCNCC3)CC2)c(C)cc1Nc1ncc(Br)c(Nc2ccc3nccnc3c2P(C)(C)=O)n1]

C116-3 (25 mg, 0.0740 mmol) and C183-1 (or C158-1) (50.2 mg, 0.0740 mmol) were dissolved in a mixture of 4.0 mL of anhydrous DCM and 0.4 mL of anhydrous methanol. CH₃COOH (4.44 mg, 0.0740 mmol, dissolved in 0.5 DCM) was added under nitrogen protection. The mixture was stirred at room temperature for 0.5 h. Then solid NaBH₃CN (6.97 mg, 0.1110 mmol) was added. The mixture was further reacted at room temperature for 2 h. LCMS and TLC showed that the reaction was completed. The mixture was purified by Prep-HPLC to afford yellow solid product C186, 23 mg, yield: 31.0%. LCMS: [M+H]⁺=1002, 1004. ¹H-NMR: (400 MHz, DMSO-d₆) δ 11.027 (s, 1H), 8.874-8.850 (m, 4H), 8.271-8.255 (m, 2H), 7.937 (d, J=9.6 Hz, 1H), 7.728 (d, J=1.2 Hz, 1H), 7.709 (d, J=1.2 Hz, 1H), 7.655-7.634 (m, 1H), 7.357 (s, 1H), 6.752 (s, 1H), 5.181 (dd, J=4.0 Hz, 13.6 Hz, 1H), 4.484 (d, J=17.6 Hz, 1H), 4.335 (d, J=17.6 Hz, 1H), 3.776 (s, 3H), 3.328-3.107 (m, 2H), 2.967-2.892 (m, 1H), 2.682-2.334 (m, 13H), 2.091-2.007 (m, 12H), 1.884-1.855 (m, 2H), 1.600-1.567 (m, 6H), 1.236 (s, 2H).

### Preparation of 3-(4-(6-(9-(4-((5-bromo-4-((5-(dimethylphosphoryl)quinoxalin-6-yl)amino)pyrimidin-2-yl)amino)-5-methoxy-2-methylphenyl)-3,9-diazaspiro[5.5]undecan-3-yl)hex-1-yn-1-yl)-1-oxoisoindolin-2-yl)piperidine-2,6-dione (C187)

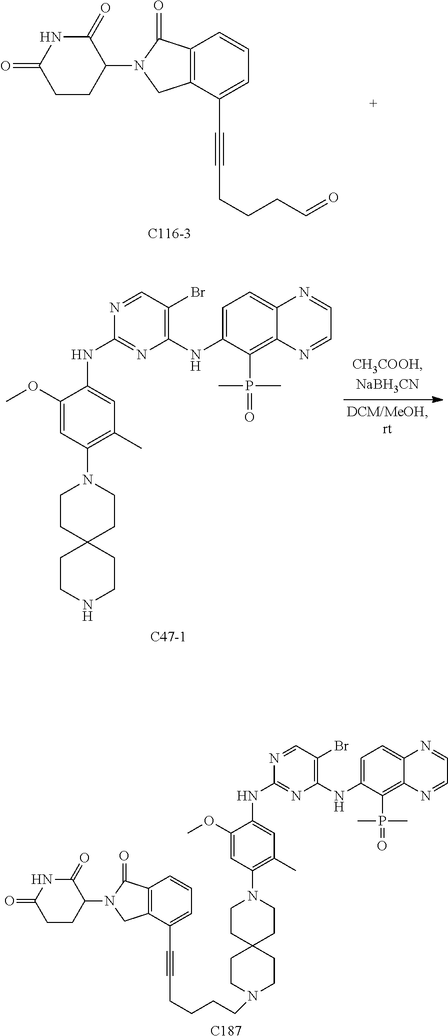 [SMILES: "C187" ; "C116-3" ; "C47-1" ; O=CCCCC#Cc1cccc2c1CN(C1CCC(=O)NC1=O)C2=O ; COc1cc(N2CCC3(CCNCC3)CC2)c(C)cc1Nc1ncc(Br)c(Nc2ccc3nccnc3c2P(C)(C)=O)n1 ; COc1cc(N2CCC3(CCN(CCCCC#Cc4cccc5c4CN(C4CCC(=O)NC4=O)C5=O)CC3)CC2)c(C)cc1Nc1ncc(Br)c(Nc2ccc3nccnc3c2P(C)(C)=O)n1]

C116-3 (25 mg, 0.0740 mmol) and C47-1 (49.1 mg, 0.0740 mmol) were dissolved in a mixture of 4.0 mL of anhydrous DCM and 0.4 mL of anhydrous methanol. CH₃COOH (4.44 mg, 0.0740 mmol, dissolved in 0.5 mL of DCM) was added under nitrogen protection. The mixture was stirred at room temperature for 0.5 h. Then solid NaBH₃CN (6.97 mg, 0.1110 mmol) was added. The mixture was further reacted at room temperature for 2 h. LCMS and TLC showed that the reaction was completed. The mixture was purified by Prep-HPLC to afford yellow solid pure product C187, 30 mg, yield: 41.1%. LCMS: [M+H]⁺=987, 989. ¹H-NMR: (400 MHz, DMSO-d₆) δ 11.026 (s, 1H), 8.864 (dd, J=2.0 Hz, 9.6 Hz, 4H), 8.265 (s, 2H), 7.940 (d, J=11.2 Hz, 1H), 7.723 (dd, J=1.2 Hz, 7.6 Hz, 1H), 7.653 (dd, J=1.2 Hz, 7.6 Hz, 1H), 7.544 (t, J=7.6 Hz, 1H), 7.327 (s, 1H), 6.815 (s, 1H), 5.181-5.135 (m, 1H), 4.483 (d, J=16.8 Hz, 1H), 4.334 (d, J=17.2 Hz, 1H), 3.783 (s, 3H), 2.933-2.821 (m, 1H), 2.807-2.797 (m, 4H), 2.621-2.577 (m, 3H), 2.503-2.335 (m, 6H), 2.081 (s, 3H), 2.040 (s, 4H), 2.004 (s, 4H), 1.600-1.528 (m, 12H).

### Preparation of 3-(4-(10-(4-(1-(4-((5-bromo-4-((5-(dimethylphosphoryl)quinoxalin-6-yl)amino)pyrimidin-2-yl)amino)-5-methoxy-2-methylphenyl)piperidin-4-yl)piperazin-1-yl)dec-1-yn-1-yl)-1-oxoisoindolin-2-yl)piperidine-2,6-dione (C188)

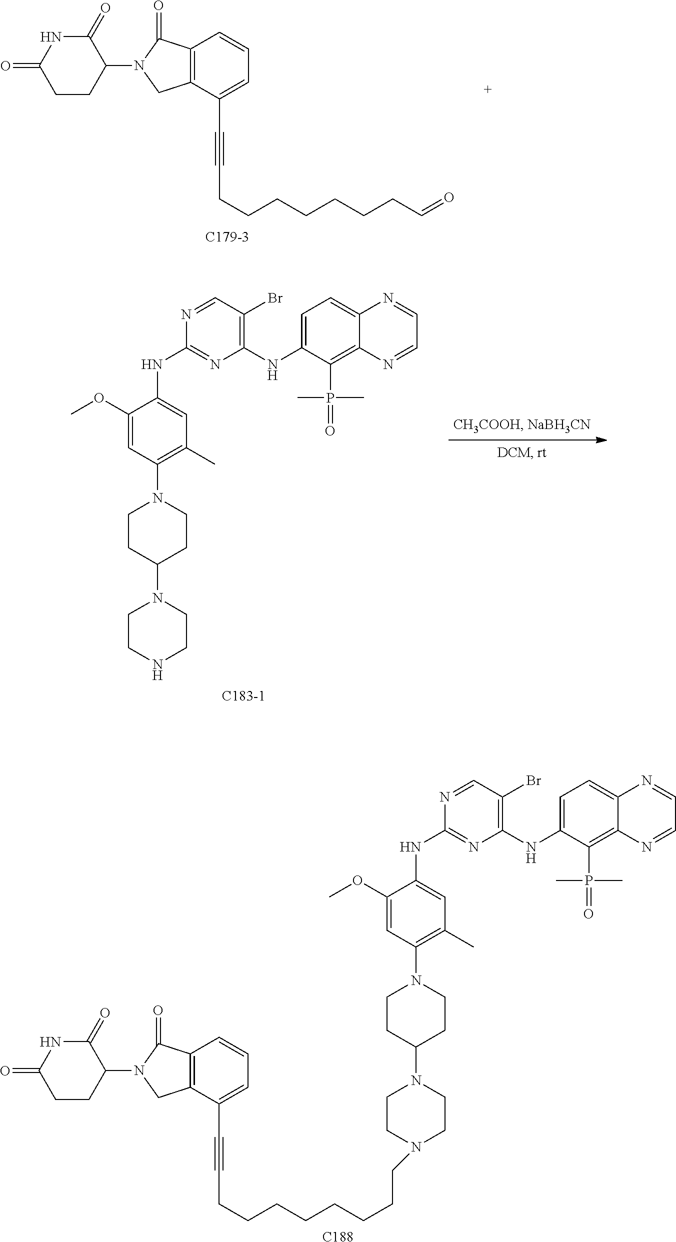 [SMILES: "C183-1" ; "C188" ; "C179-3" ; COc1cc(N2CCC(N3CCN(CCCCCCCCC#Cc4cccc5c4CN(C4CCC(=O)NC4=O)C5=O)CC3)CC2)c(C)cc1Nc1ncc(Br)c(Nc2ccc3nccnc3c2P(C)(C)=O)n1 ; O=CCCCCCCCC#Cc1cccc2c1CN(C1CCC(=O)NC1=O)C2=O ; COc1cc(N2CCC(N3CCNCC3)CC2)c(C)cc1Nc1ncc(Br)c(Nc2ccc3nccnc3c2P(C)(C)=O)n1]

C179-3 (30 mg, 0.076 mmol) and C183-1 (or C158-1) (43.9 mg, 0.0647 mmol) were dissolved in 8.0 mL of anhydrous DCM. CH₃COOH (4.56 mg, 0.076 mmol, dissolved in 0.5 mL of DCM) was added under nitrogen protection. The mixture was stirred at room temperature for 0.5 h. Then solid NaBH₃CN (7.16 mg, 0.114 mmol) was added. The mixture was further reacted at room temperature for 2 h. LCMS and TLC showed that the reaction was completed. The mixture was directly diluted with 20 mL of DCM, and washed successively with saturated NH₄Cl (1×10 mL) and saturated brine (1×10 mL). The organic layer was dried with anhydrous sodium sulfate, and filtered with suction. The filtrate was concentrated under reduced pressure to afford a crude product, which was purified by Prep-HPLC to afford yellow solid pure product C188 (19.5 mg, yield: 24.4%). LCMS: [M+H]⁺=1058, 1060.

### Preparation of 3-(4-(10-(9-(4-((5-bromo-4-((5-(dimethylphosphoryl)quinoxalin-6-yl)amino)pyrimidin-2-yl)amino)-5-methoxy-2-methylphenyl)-3,9-diazaspiro[5.5]undecan-3-yl)dec-1-yn-1-yl)-1-oxoisoindolin-2-yl)piperidine-2,6-dione (C189)

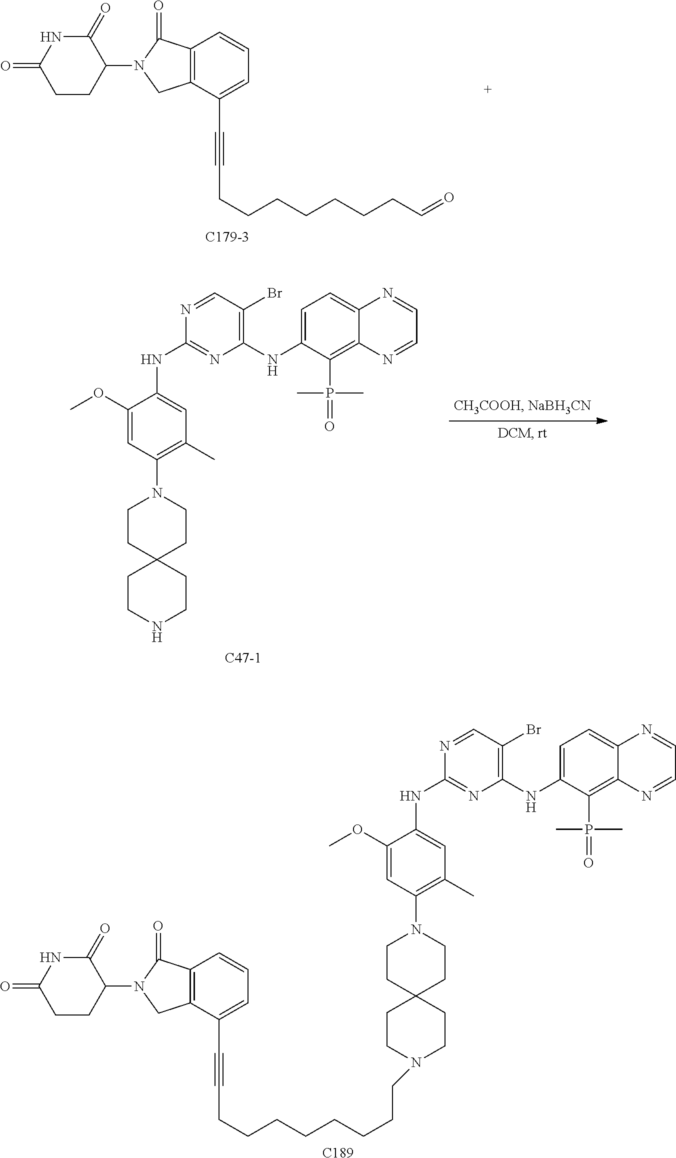 [SMILES: "C189" ; "C179-3" ; "C47-1" ; COc1cc(N2CCC3(CCN(CCCCCCCCC#Cc4cccc5c4CN(C4CCC(=O)NC4=O)C5=O)CC3)CC2)c(C)cc1Nc1ncc(Br)c(Nc2ccc3nccnc3c2P(C)(C)=O)n1 ; O=CCCCCCCCC#Cc1cccc2c1CN(C1CCC(=O)NC1=O)C2=O ; COc1cc(N2CCC3(CCNCC3)CC2)c(C)cc1Nc1ncc(Br)c(Nc2ccc3nccnc3c2P(C)(C)=O)n1]

C179-3 (30 mg, 0.076 mmol) and C47-1 (40.4 mg, 0.0608 mmol) were dissolved in 5.0 mL of anhydrous DCM. CH₃COOH (4.56 mg, 0.076 mmol, dissolved in 0.5 mL of DCM) was added under nitrogen protection. The mixture was stirred at room temperature for 0.5 h. Then solid NaBH₃CN (7.16 mg, 0.114 mmol) was added. The mixture was further reacted at room temperature for 2 h. LCMS and TLC showed that the reaction was completed. The mixture was purified by Prep-HPLC to afford yellow solid pure product C189 (25 mg, yield: 31.5%). LCMS: [M+H]⁺=1043, 1045.

### Preparation of 3-(5-(7-(4-(1-(4-((5-chloro-4-((2-(dimethylphosphoryl)phenyl)amino)pyrimidin-2-yl)amino)-3-methoxyphenyl)piperidin-4-yl)piperazin-1-yl)hept-1-yn-1-yl)-1-oxoisoindolin-2-yl)piperidine-2,6-dione (C190)

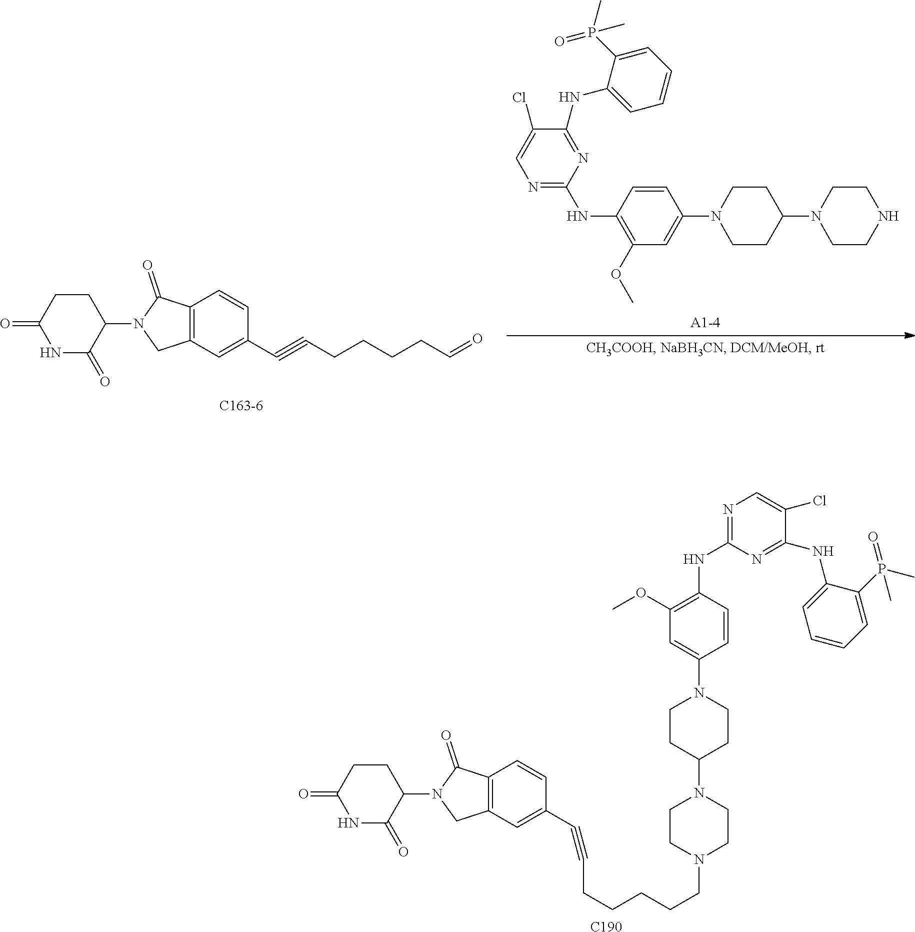 [SMILES: O=CCCCCC#Cc1ccc2c(c1)CN(C1CCC(=O)NC1=O)C2=O ; COc1cc(N2CCC(N3CCNCC3)CC2)ccc1Nc1ncc(Cl)c(Nc2ccccc2P(C)(C)=O)n1 ; COc1cc(N2CCC(N3CCN(CCCCCC#Cc4ccc5c(c4)CN(C4CCC(=O)NC4=O)C5=O)CC3)CC2)ccc1Nc1ncc(Cl)c(Nc2ccccc2P(C)(C)=O)n1 ; "C190"]

Compound C163-6 (35 mg, 0.099 mmol) and compound A1-4 (62 mg, 0.109 mmol) were dissolved in 3 mL of dichloromethane/methanol (2/1), and 1 drop of glacial acetic acid was added dropwise. The mixture was stirred at room temperature under nitrogen protection for 1 h. Then sodium cyanobohydride (6.2 mg, 0.099 mmol) was added. The mixture was further stirred for 1 h. The solvent was removed under reduced pressure below 40° C., and the crude product was purified by Flash chromatography to afford off-white solid compound C190 (15 mg, yield: 16.7%). LCMS: [M+H]⁺=906.

### Preparation of 3-(5-(7-(9-(4-((5-bromo-4-((5-(dimethylphosphoryl)quinoxalin-6-yl)amino)pyrimidin-2-yl)amino)-5-methoxy-2-methylphenyl)-3,9-diazaspiro[5.5]undecan-3-yl)hept-1-yn-1-yl)-1-oxoisoindolin-2-yl)piperidine-2,6-dione (C191)

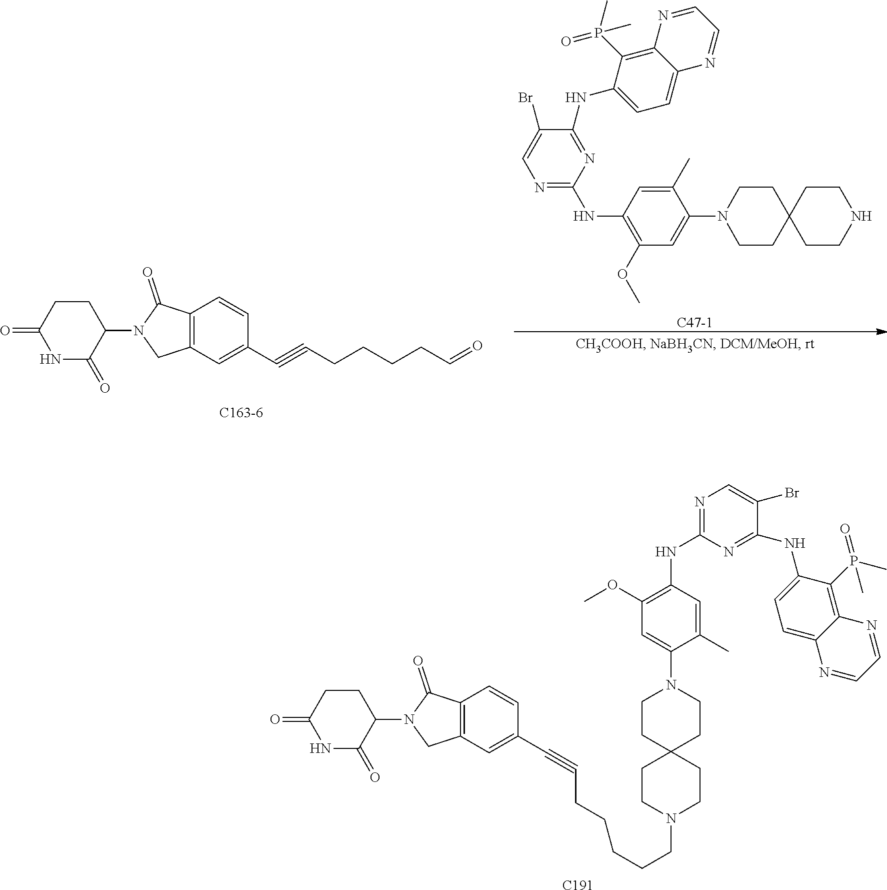 [SMILES: "C47-1" ; O=CCCCCC#Cc1ccc2c(c1)CN(C1CCC(=O)NC1=O)C2=O ; COc1cc(N2CCC3(CCNCC3)CC2)c(C)cc1Nc1ncc(Br)c(Nc2ccc3nccnc3c2P(C)(C)=O)n1 ; COc1cc(N2CCC3(CCN(CCCCCC#Cc4ccc5c(c4)CN(C4CCC(=O)NC4=O)C5=O)CC3)CC2)c(C)cc1Nc1ncc(Br)c(Nc2ccc3nccnc3c2P(C)(C)=O)n1 ; "C191"]

Compound C163-6 (35 mg, 0.099 mmol) and compound C47-1 (73 mg, 0.109 mmol) were dissolved in 3 mL of dichloromethane/methanol (2/1), and 1 drop of glacial acetic acid was added dropwise. The mixture was stirred at room temperature under nitrogen protection for 1 h. Then sodium cyanobohydride (6.2 mg, 0.099 mmol) was added. The mixture was further stirred for 1 h. The solvent was removed under reduced pressure below 40° C. The crude product was purified by Flash chromatography to afford yellow solid compound C191 (20 mg, yield: 20.2%). LCMS: [M+H]⁺=1001, 1003.

### Preparation of 3-(5-(6-(4-(1-(4-((5-chloro-4-((2-(dimethylphosphoryl)phenyl)amino)pyrimidin-2-yl)amino)-3-methoxyphenyl)piperidin-4-yl)piperazin-1-yl)hex-1-yn-1-yl)-1-oxoisoindolin-2-yl)piperidine-2,6-dione (C192)

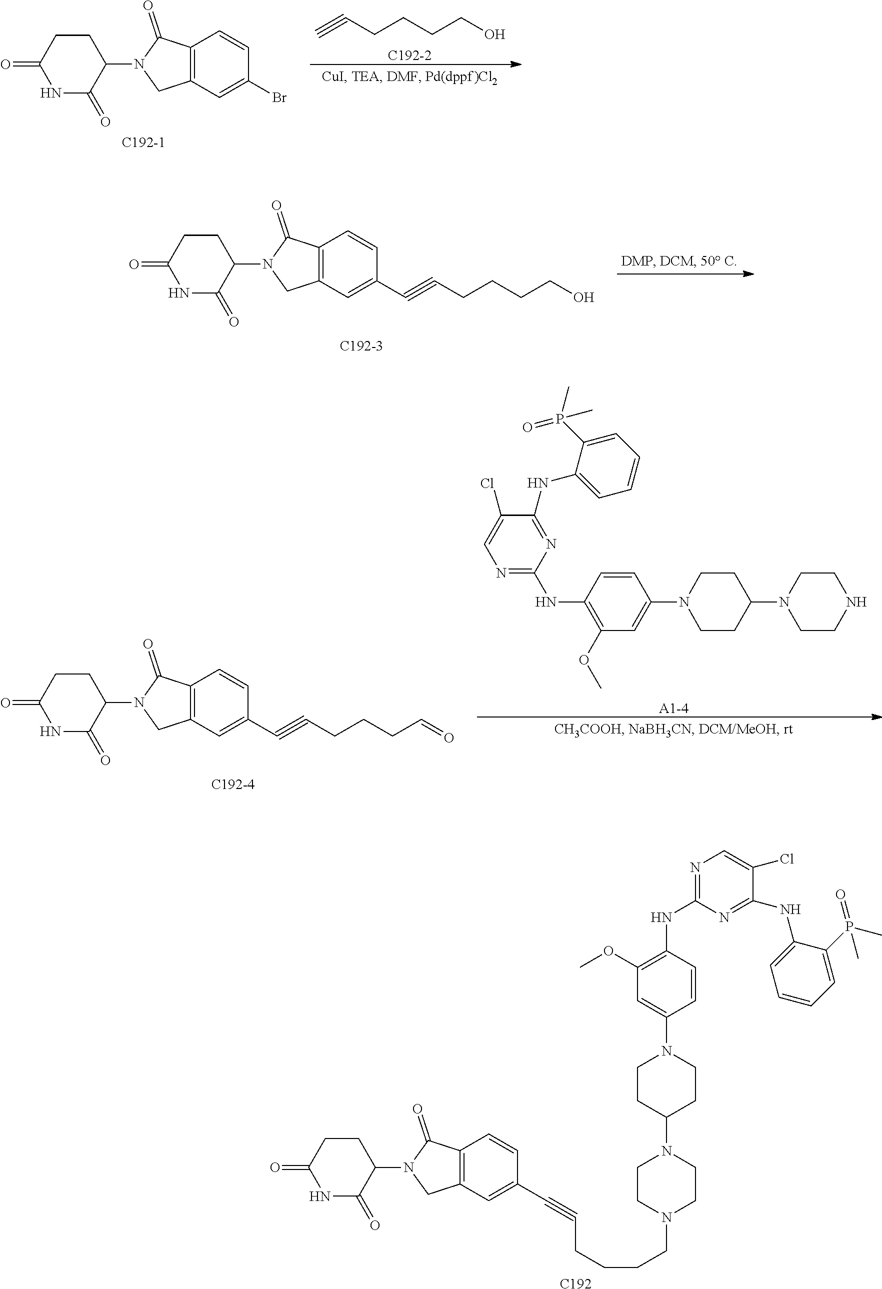

### Step 1

Compound C192-1 (100 mg, 0.31 mmol), compound C192-2 (76 mg, 0.78 mmol), cuprous iodide (12 mg, 0.06 mmol), Pd(dppf)Cl₂ (87 mg, 0.12 mmol) and triethylamine (94 mg, 0.93 mmol) were dissolved in 4 mL of anhydrous DMF. The mixture was reacted with stirring at 70° C. under nitrogen protection for 5 h. The solvent was removed under reduced pressure. The crude product was purified by thin layer chromatography to afford off-white solid compound C192-3 (78 mg, yield: 72.2%). LCMS: [M+H]⁺=341.

### Step 2

Compound C192-3 (39 mg, 0.115 mmol) and Dess-Martin reagent (73 mg, 0.172 mmol) were dissolved in 15 mL of dichloromethane and stirred at 50° C. for 2 h. The mixture was cooled to room temperature, and quenched with saturated sodium bicarbonate aqueous solution. The organic phase was separated, and the aqueous phase was extracted with dichloromethane for three times. The organic phases were combined, dried with anhydrous sodium sulfate, and filtered. The filtrate was concentrated, and the crude product was purified by TLC to afford a white solid compound C192-4 (30 mg, yield: 77.5%). LCMS: [M+H]⁺=339.

### Step 3

Compound C192-4 (26.6 mg, 0.079 mmol) and compound A1-4 (49 mg, 0.087 mmol) were dissolved in 3 mL of dichloromethane/methanol (2/1), and 1 drop of glacial acetic acid was added dropwise. The mixture was stirred under nitrogen protection at room temperature for 1 h. Then sodium cyanobohydride (5 mg, 0.079 mmol) was added. The mixture was further stirred for 1 h. The solvent was removed under reduced pressure below 40° C., and the crude product was purified by Flash chromatography to afford off-white solid compound C192 (10 mg, yield: 14.3%). LCMS: [M+H]⁺=892.

### Preparation of 3-(5-(3-(2-(4-(1-(4-((5-chloro-4-((2-(dimethylphosphoryl)phenyl)amino)pyrimidin-2-yl)amino)-3-methoxyphenyl)piperidin-4-yl)piperazin-1-yl)ethoxy)prop-1-yn-1-yl)-1-oxoisoindolin-2-yl)piperidine-2,6-dione (C193)

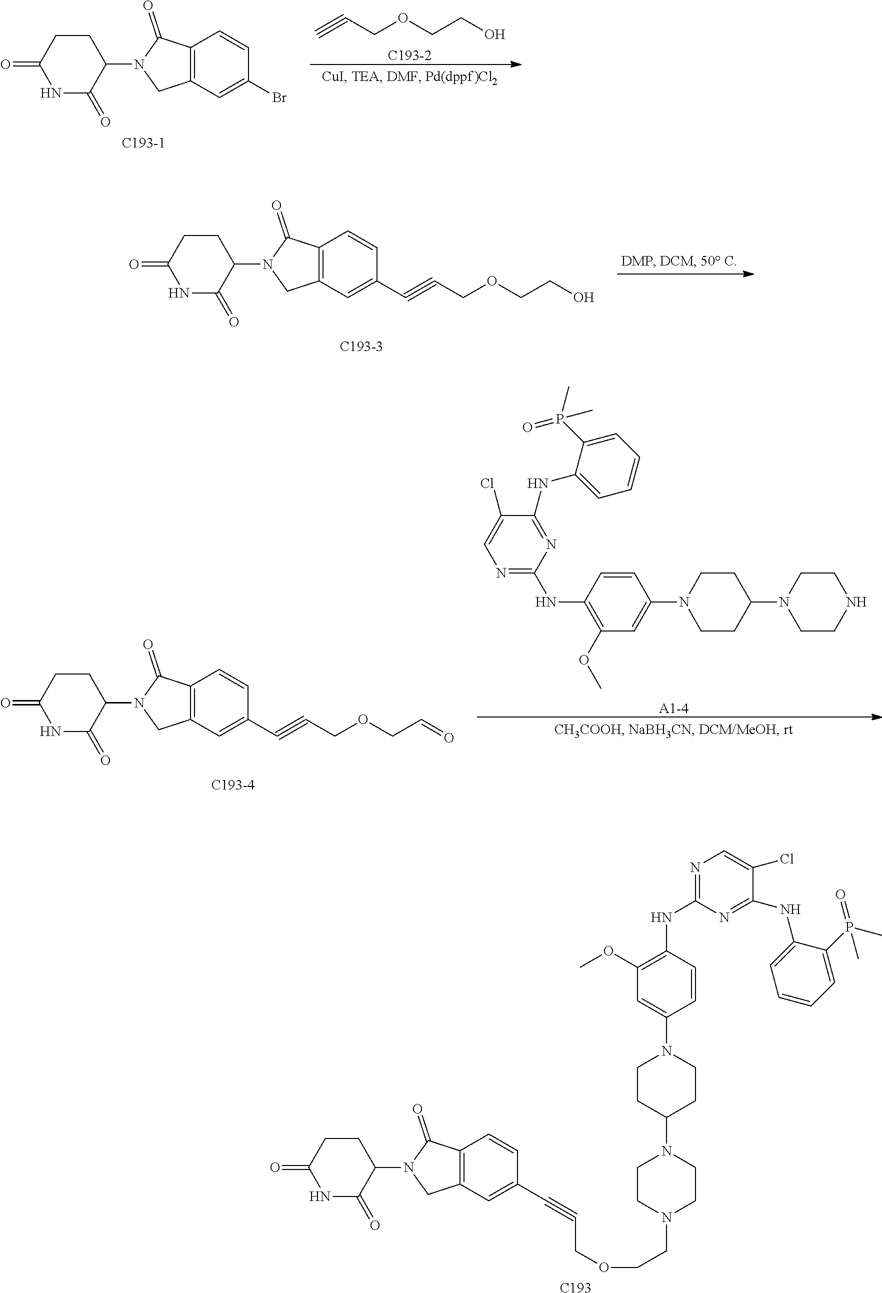

### Step 1

Compound C193-1 (or C192-1) (100 mg, 0.31 mmol), compound C193-2 (78 mg, 0.78 mmol), cuprous iodide (12 mg, 0.06 mmol), Pd(dppf)Cl₂ (87 mg, 0.12 mmol) and triethylamine (94 mg, 0.93 mmol) were dissolved in 4 mL of anhydrous DMF. The mixture was reacted with stirring at 70° C. under nitrogen protection for 5 h. The solvent was removed under reduced pressure, and the crude product was purified by thin layer chromatography to afford off-white solid compound C193-3 (70 mg, yield: 66.0%). LCMS: [M+H]⁺=343.

### Step 2

Compound C193-3 (23.2 mg, 0.068 mmol) and Dess-Martin reagent (43.1 mg, 0.102 mmol) were dissolved in 15 mL of dichloromethane and stirred at 50° C. for 2 h. The mixture was cooled to room temperature, and quenched with saturated sodium bicarbonate aqueous solution. The organic phase was separated, and the aqueous phase was extracted with dichloromethane for three times. The organic phases were combined, dried with anhydrous sodium sulfate, and filtered. The filtrate was concentrated, and the crude product was purified by TLC to afford a white solid compound C193-4 (20 mg, yield: 87.0%). LCMS: [M+H]⁺=341.

### Step 3

Compound C193-4 (20 mg, 0.059 mmol) and compound A1-4 (37 mg, 0.065 mmol) were dissolved in 3 mL of dichloromethane/methanol (2/1), and 1 drop of glacial acetic acid was added dropwise. The mixture was stirred at room temperature under nitrogen protection for 1 h. Then sodium cyanobohydride (3.7 mg, 0.059 mmol) was added. The mixture was further stirred for 1 h. The solvent was removed under reduced pressure below 40° C. The crude product was purified by Flash chromatography to afford off-white solid compound C193 (15 mg, yield: 28.8%). LCMS: [M+H]⁺=894.

### Preparation of 3-(5-(8-(4-(1-(4-((5-chloro-4-((2-(dimethylphosphoryl)phenyl)amino)pyrimidin-2-yl)amino)-3-methoxyphenyl)piperidin-4-yl)piperazin-1-yl)oct-1-yn-1-yl)-1-oxoisoindolin-2-yl)piperidine-2,6-dione (C194)

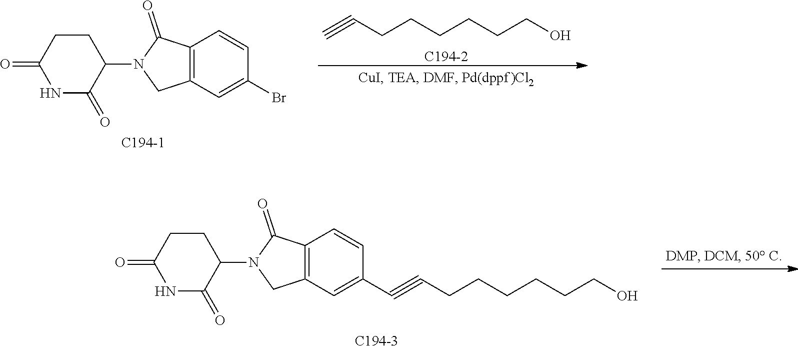

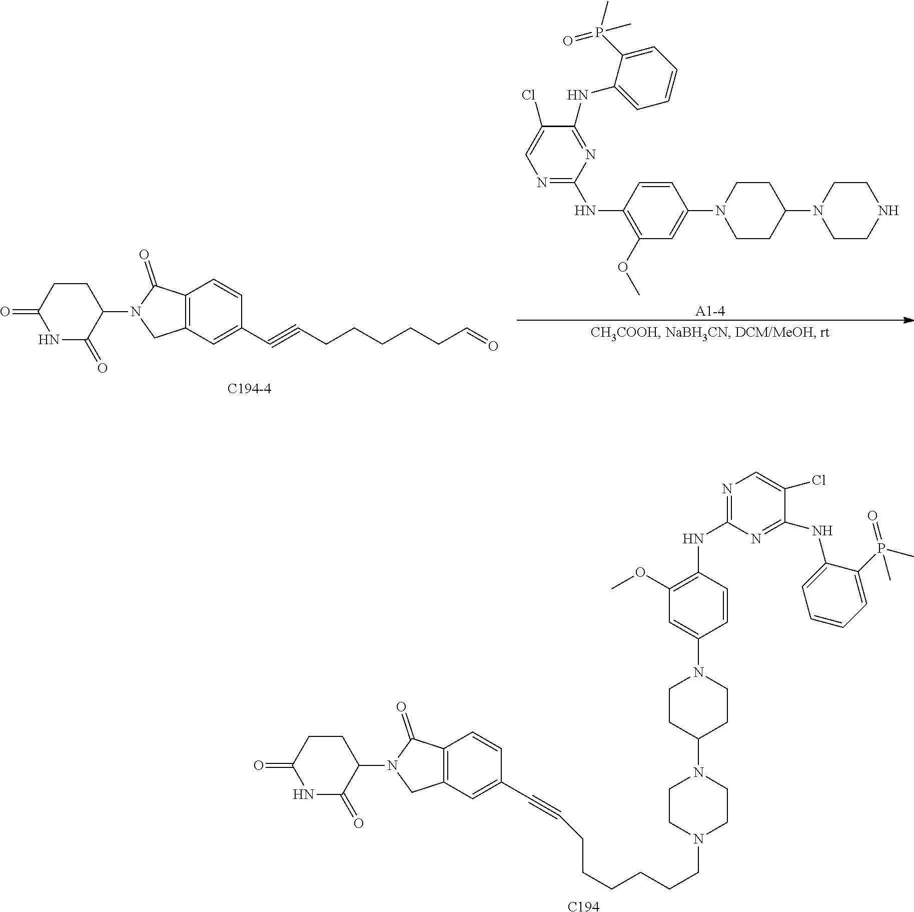 [SMILES: "C194" ; "C194-4" ; O=CCCCCCC#Cc1ccc2c(c1)CN(C1CCC(=O)NC1=O)C2=O ; COc1cc(N2CCC(N3CCN(CCCCCCC#Cc4ccc5c(c4)CN(C4CCC(=O)NC4=O)C5=O)CC3)CC2)ccc1Nc1ncc(Cl)c(Nc2ccccc2P(C)(C)=O)n1 ; COc1cc(N2CCC(N3CCNCC3)CC2)ccc1Nc1ncc(Cl)c(Nc2ccccc2P(C)(C)=O)n1 ; "A1-4"]

### Step 1

Compound C194-1 (or C192-1) (100 mg, 0.31 mmol), compound C194-2 (98 mg, 0.78 mmol), cuprous iodide (12 mg, 0.06 mmol), Pd(dppf)Cl₂ (87 mg, 0.12 mmol) and triethylamine (94 mg, 0.93 mmol) were dissolved in 4 mL of anhydrous DMF. The mixture was reacted with stirring at 70° C. under nitrogen protection for 5 h. The solvent was removed under reduced pressure. The crude product was purified by thin layer chromatography to afford off-white solid compound C194-3 (68 mg, yield: 59.6%). LCMS: [M+H]⁺=369.

### Step 2

Compound C194-3 (27 mg, 0.073 mmol) and Dess-Martin reagent (46.7 mg, 0.110 mmol) were dissolved in 16 mL of dichloromethane and stirred at 50° C. for 2 h. The mixture was cooled to room temperature, and quenched with saturated sodium bicarbonate aqueous solution. The organic phase was separated, and the aqueous phase was extracted with dichloromethane for three times. The organic phases were combined, dried with anhydrous sodium sulfate, and filtered. The filtrate was concentrated, and the crude product was purified by TLC to afford a white solid compound C194-4 (20.6 mg, yield: 76.6%). LCMS: [M+H]⁺=367.

### Step 3

Compound C194-4 (20.6 mg, 0.056 mmol) and compound A1-4 (35 mg, 0.062 mmol) were dissolved in 3 mL of dichloromethane/methanol (2/1), and 1 drop of glacial acetic acid was added dropwise. The mixture was stirred at room temperature under nitrogen protection for 1 h. Then sodium cyanobohydride (3.5 mg, 0.056 mmol) was added. The mixture was further stirred for 1 h. The solvent was removed under reduced pressure below 40° C., and the crude product was purified by Flash chromatography to afford off-white solid compound C194 (15 mg, yield: 29.0%). LCMS: [M+H]⁺=920.

### Preparation of 3-(5-(9-(4-(1-(4-((5-chloro-4-((2-(dimethylphosphoryl)phenyl)amino)pyrimidin-2-yl)amino)-3-methoxyphenyl)piperidin-4-yl)piperazin-1-yl)non-1-yn-1-yl)-1-oxoisoindolin-2-yl)piperidine-2,6-dione (C195)

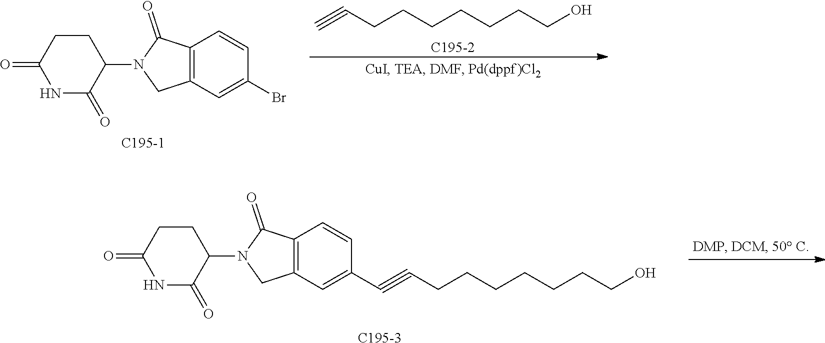

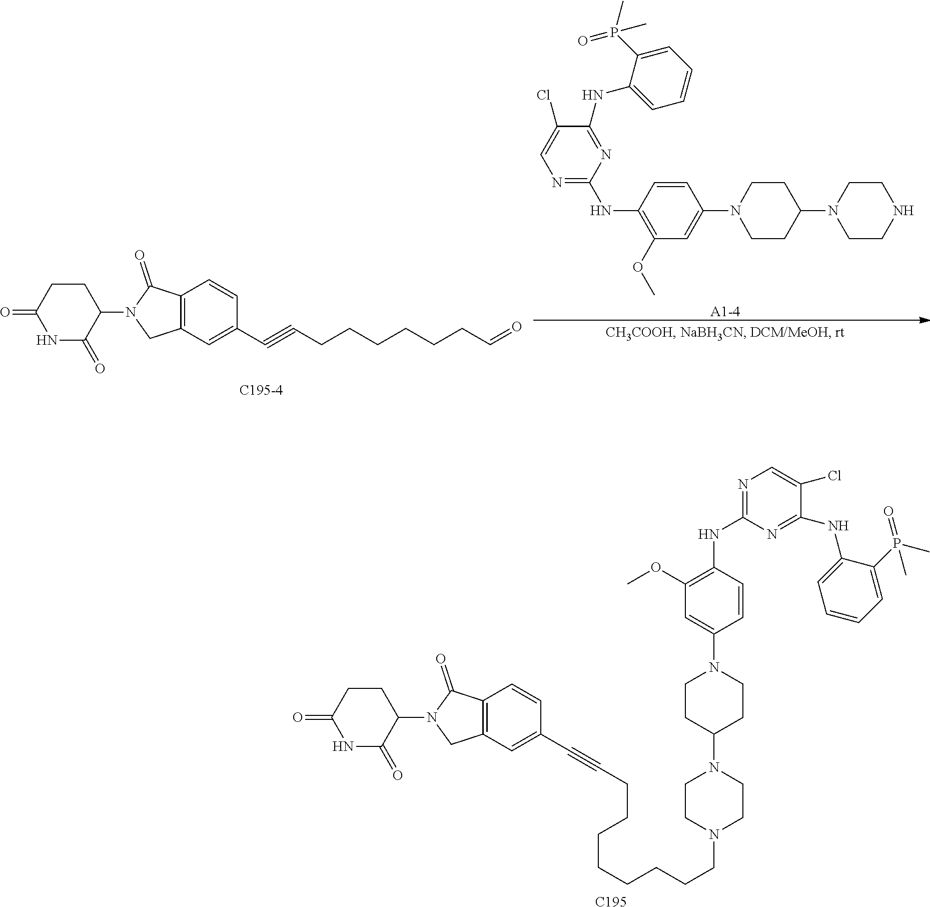 [SMILES: "C195" ; "C195-4" ; O=CCCCCCCC#Cc1ccc2c(c1)CN(C1CCC(=O)NC1=O)C2=O ; COc1cc(N2CCC(N3CCN(CCCCCCCCC#Cc4ccc5c(c4)CN(C4CCC(=O)NC4=O)C5=O)CC3)CC2)ccc1Nc1ncc(Cl)c(Nc2ccccc2P(C)(C)=O)n1 ; COc1cc(N2CCC(N3CCNCC3)CC2)ccc1Nc1ncc(Cl)c(Nc2ccccc2P(C)(C)=O)n1 ; "A1-4"]

### Step 1

Compound C195-1 (orC192-1) (100 mg, 0.31 mmol), compound C195-2 (109 mg, 0.78 mmol), cuprous iodide (12 mg, 0.06 mmol), Pd(dppf)Cl₂ (87 mg, 0.12 mmol) and triethylamine (94 mg, 0.93 mmol) were dissolved in 4 mL of anhydrous DMF. The mixture was reacted with stirring at 70° C. under nitrogen protection for 5 h. The solvent was removed under reduced pressure, and the crude product was purified by thin layer chromatography to afford off-white solid compound C195-3 (72 mg, yield: 61.0%). LCMS: [M+H]⁺=383.

### Step 2

Compound C195-3 (23 mg, 0.060 mmol) and Dess-Martin reagent (38.3 mg, 0.090 mmol) were dissolved in 16 mL of dichloromethane and stirred at 50° C. for 2 h. The mixture was cooled to room temperature, and quenched with saturated sodium bicarbonate aqueous solution. The organic phase was separated, and the aqueous phase was extracted with dichloromethane for three times. The organic phases were combined, dried with anhydrous sodium sulfate, and filtered. The filtrate was concentrated, and the crude product was purified by TLC to afford white solid compound C195-4 (20 mg, yield: 87.0%). LCMS: [M+H]⁺=381.

### Step 3

Compound C195-4 (20 mg, 0.053 mmol) and A1-4 (33 mg, 0.058 mmol) were dissolved in 3 mL of dichloromethane/methanol (2/1), and 1 drop of glacial acetic acid was added dropwise. The mixture was stirred at room temperature under nitrogen protection for 1 h. Then sodium cyanobohydride (3.5 mg, 0.053 mmol) was added. The mixture was further stirred for 1 h. The solvent was removed under reduced pressure below 40° C., and the crude product was purified by Flash chromatography to afford off-white solid compound C195 (15 mg, yield: 30.6%). LCMS: [M+H]⁺=934.

### Preparation of 3-(5-(10-(4-(1-(4-((5-chloro-4-((2-(dimethylphosphoryl)phenyl)amino)pyrimidin-2-yl)amino)-3-methoxyphenyl)piperidin-4-yl)piperazin-1-yl)dec-1-yn-1-yl)-1-oxoisoindolin-2-yl)piperidine-2,6-dione (C196)

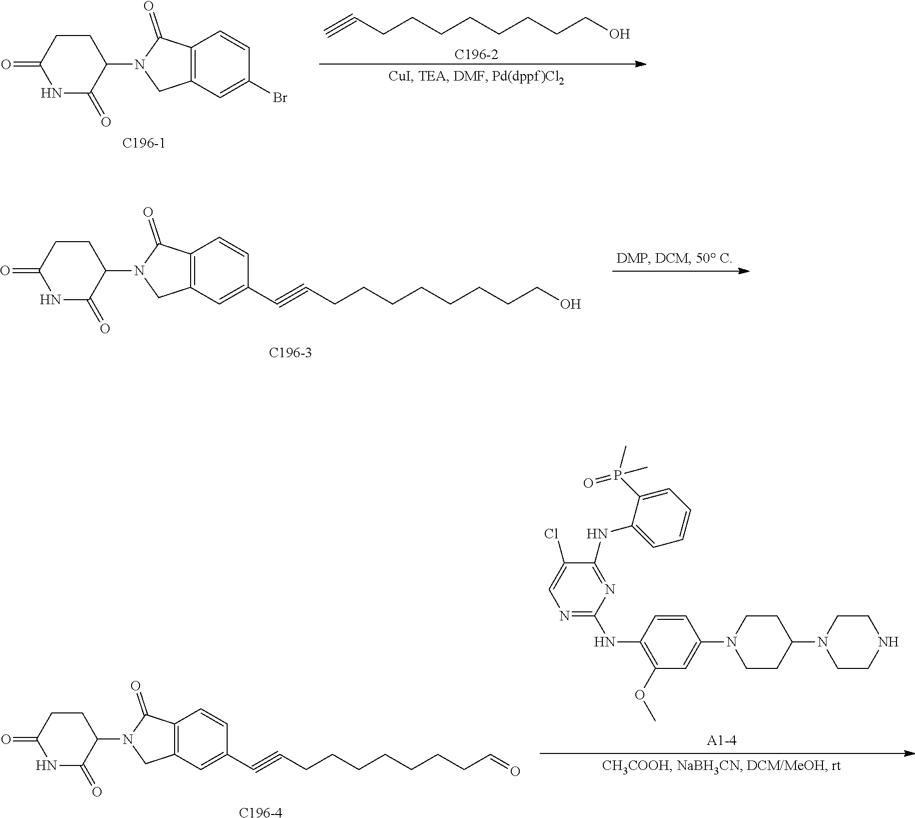

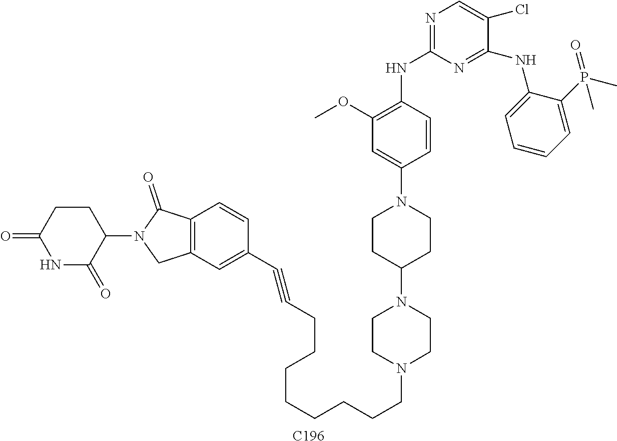 [SMILES: COc1cc(N2CCC(N3CCN(CCCCCCCCC#Cc4ccc5c(c4)CN(C4CCC(=O)NC4=O)C5=O)CC3)CC2)ccc1Nc1ncc(Cl)c(Nc2ccccc2P(C)(C)=O)n1 ; "C196"]

### Step 1

Compound C196-1 (orC192-1) (100 mg, 0.31 mmol), compound C196-2 (120 mg, 0.78 mmol), cuprous iodide (12 mg, 0.06 mmol), Pd(dppf)Cl₂ (87 mg, 0.12 mmol) and triethylamine (94 mg, 0.93 mmol) were dissolved in 4 mL of anhydrous DMF. The mixture was reacted with stirring at 70° C. under nitrogen protection for 5 h. The solvent was removed under reduced pressure, and the crude product was purified by thin layer chromatography to afford off-white solid compound C196-3 (65 mg, yield: 52.8%). LCMS: [M+H]⁺=397.

### Step 2

Compound C195-3 (23.9 mg, 0.060 mmol) and Dess-Martin reagent (38.4 mg, 0.090 mmol) were dissolved in 15 mL of dichloromethane and stirred at 50° C. for 2 h. The mixture was cooled to room temperature, and quenched with saturated sodium bicarbonate aqueous solution. The organic phase was separated, and the aqueous phase was extracted with dichloromethane for three times. The organic phases were combined, dried with anhydrous sodium sulfate, and filtered. The filtrate was concentrated, and the crude product was purified by TLC to afford a white solid compound C196-4 (20 mg, yield: 84.0%). LCMS: [M+H]⁺=395.

### Step 3

Compound C196-4 (20 mg, 0.051 mmol) and compound A1-4 (32 mg, 0.056 mmol) were dissolved in 3 mL of dichloromethane/methanol (2/1), and 1 drop of glacial acetic acid was added dropwise. The mixture was stirred at room temperature under nitrogen protection for 1 h. Then sodium cyanobohydride (3.2 mg, 0.051 mmol) was added. The mixture was further stirred for 1 h. The solvent was removed under reduced pressure below 40° C., and the crude product was purified by Flash chromatography to afford off-white solid compound C196 (10 mg, yield: 20.8%). LCMS: [M+H]⁺=948.

### Preparation of 3-(4-(3-(2-(4-(1-(4-((5-chloro-4-((2-(dimethylphosphoryl)phenyl)amino)pyrimidin-2-yl)amino)-3-methoxyphenyl)piperidin-4-yl)piperazin-1-yl)ethoxy)prop-1-yn-1-yl)-1-oxo-6-(trifluoromethyl)isoindolin-2-yl)piperidine-2,6-dione (C200)

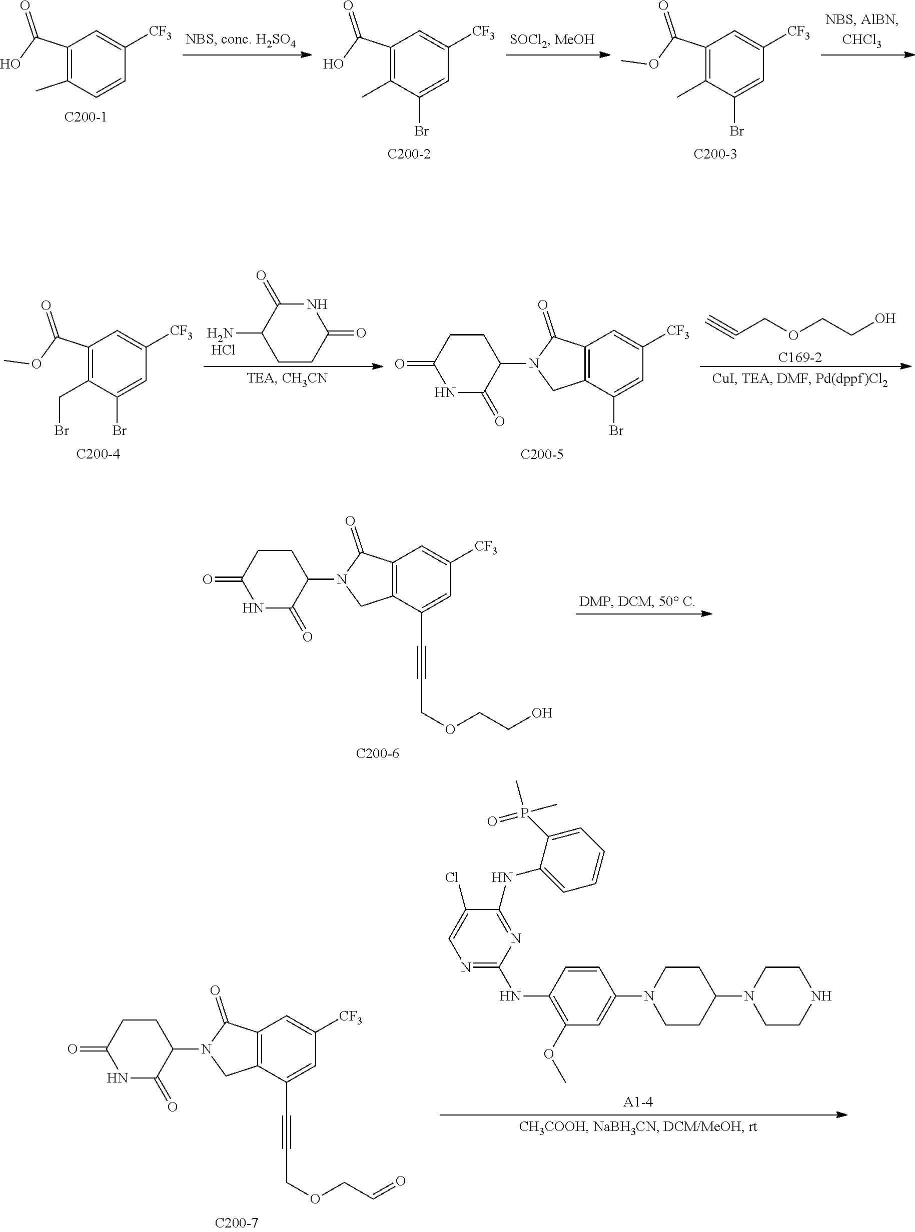

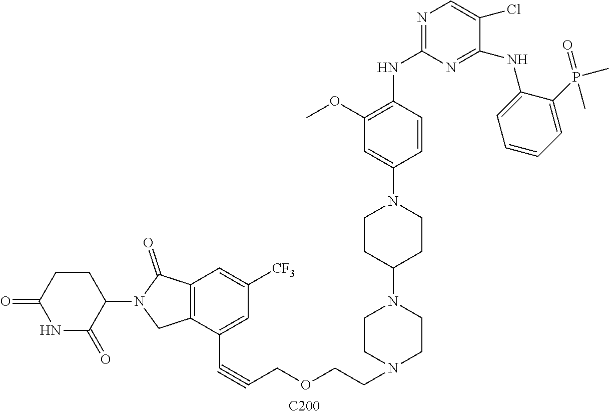 [SMILES: COc1cc(N2CCC(N3CCN(CCOCC#Cc4cc(C(F)(F)F)cc5c4CN(C4CCC(=O)NC4=O)C5=O)CC3)CC2)ccc1Nc1ncc(Cl)c(Nc2ccccc2P(C)(C)=O)n1 ; "C200"]

### Step 1

At 0° C., compound C200-1 (2 g, 9.8 mmol) and NBS (1.63 g, 9.16 mmol) were added portionwise with stirring to concentrated sulfuric acid (20 mL). The mixture was reacted with stirring at this temperature under nitrogen protection for 3 h. The mixture was warmed to room temperature and stirred overnight. The mixture was slowly poured into ice water, and extracted with ethyl acetate for three times. The organic phases were combined, washed once with 0.5M hydrochloric acid and once with saturated brine, dried with anhydrous sodium sulfate, and filtered. The filtrate was concentrated, and the crude product was purified by Flash chromatography to afford compound C200-2 (1.3 g, yield: 47.1%). LCMS: [M+H]⁺=283, 285.

### Step 2

At 0° C., thionyl chloride (2 mL, 25.57 mmol) was added dropwise to a solution of compound C200-2 (1.3 g, 4.61 mmol) in 20 mL of methanol. The mixture was heated and stirred at 70° C. overnight. The mixture was cooled to room temperature, and concentrated under reduced pressure. The crude product was purified by Flash chromatography to afford compound C200-3 (1.25 g, yield: 91.9%).

### Step 3

Compound C200-3 (1.25 g, 4.22 mmol), NBS (857 mg, 4.81 mmol), and AIBN (69 mg, 0.42 mmol) were dissolved in 20 mL of chloroform. The mixture was refluxed at 95° C. under nitrogen protection overnight. The solvent was removed under reduced pressure. The crude product was purified by Flash chromatography to afford white solid compound C200-4 (1.3 g, yield: 82.3%).

### Step 4

Compound C200-4 (1.33 g, 3.56 mmol), 3-aminopiperidine-2,6-dione hydrochloride (759 mg, 4.62 mmol), and triethylamine (467 mg, 4.62 mmol) were dissolved in 15 mL of acetonitrile. The mixture was stirred at 80° C. under nitrogen protection overnight. The mixture was cooled to room temperature. The solvent was removed under reduced pressure, and the crude product was purified by Flash chromatography to afford white solid compound C200-5 (790 mg, yield: 56.9%). LCMS: [M+H]⁺=391, 393.

### Step 5

Compound C200-5 (100 mg, 0.256 mmol), compound C169-2 (64 mg, 0.641 mmol), cuprous iodide (4.9 mg, 0.026 mmol), Pd(dppf)Cl₂ (18.8 mg, 0.026 mmol) and triethylamine (77.7 mg, 0.769 mmol) were dissolved in 3 mL of anhydrous DMF. The mixture was stirred at 70° C. under nitrogen protection overnight. The solvent was removed under reduced pressure, and the crude product was purified by thin layer chromatography to afford compound C200-6 (100 mg, yield: 95.2%). LCMS: [M+H]⁺=411.

### Step 6

Compound C200-6 (126 mg, 0.307 mmol) and Dess-Martin reagent (261 mg, 0.615 mmol) were dissolved in 10 mL of dichloromethane and stirred at 50° C. for 2 h. The mixture was cooled to room temperature, and saturated sodium bicarbonate aqueous solution and saturated sodium thiosulfate aqueous solution were added successively and stirred for 5 minutes respectively to quench the reaction. The organic phase was separated, and the aqueous phase was extracted with dichloromethane for three times. The organic phases were combined, dried with anhydrous sodium sulfate, and filtered. The filtrate was concentrated, and the crude product was purified by TLC to afford a white solid compound C200-7 (44 mg, yield: 35.2%). LCMS: [M+H]⁺=409.

### Step 7

Compound C200-7 (22 mg, 0.054 mmol) and compound A1-4 (21.5 mg, 0.038 mmol) were dissolved in 1.5 mL of dichloromethane/methanol (2/1, v/v), and glacial acetic acid (3.2 mg, 0.054 mmol) was added dropwise. The mixture was stirred at room temperature under nitrogen protection for 1 h. Then sodium cyanobohydride (2.4 mg, 0.038 mmol) was added. The mixture was further stirred for 1 h. The solvent was removed under reduced pressure below 40° C., and the crude product was purified by Flash chromatography to afford off-white solid compound C200 (18 mg, yield: 34.7%). LCMS: [M+H]⁺=962. ¹H NMR (400 MHz, DMSO-d₆) δ 11.17 (s, 1H), 11.04 (s, 1H), 8.47 (s, 1H), 8.11 (d, 1H), 8.06 (s, 1H), 8.05 (d, 2H), 7.57-7.49 (m, 1H), 7.38 (d, 1H), 7.33 (d, 1H), 7.09 (t, 1H), 6.62 (d, 1H), 6.47 (dd, 1H), 5.34-5.30 (m, 1H), 5.19 (dd, 1H), 4.62 (d, 1H), 4.48 (s, 3H), 3.76 (s, 3H), 3.73 (s, 1H), 3.67 (t, 3H), 3.29 (m, 3H), 2.93 (m, 1H), 2.70-2.61 (m, 4H), 2.52 (d, 3H), 2.01 (m, 4H), 1.84 (d, 2H), 1.76 (d, 6H), 1.55-1.45 (m, 3H).

### Preparation of 3-(6-chloro-4-(7-(4-(1-(4-((5-chloro-4-((2-(dimethylphosphoryl)phenyl)amino)pyrimidin-2-yl)amino)-3-methoxyphenyl)piperidin-4-yl)piperazin-1-yl)hept-1-yn-1-yl)-1-oxoisoindolin-2-yl)piperidine-2,6-dione (C202)

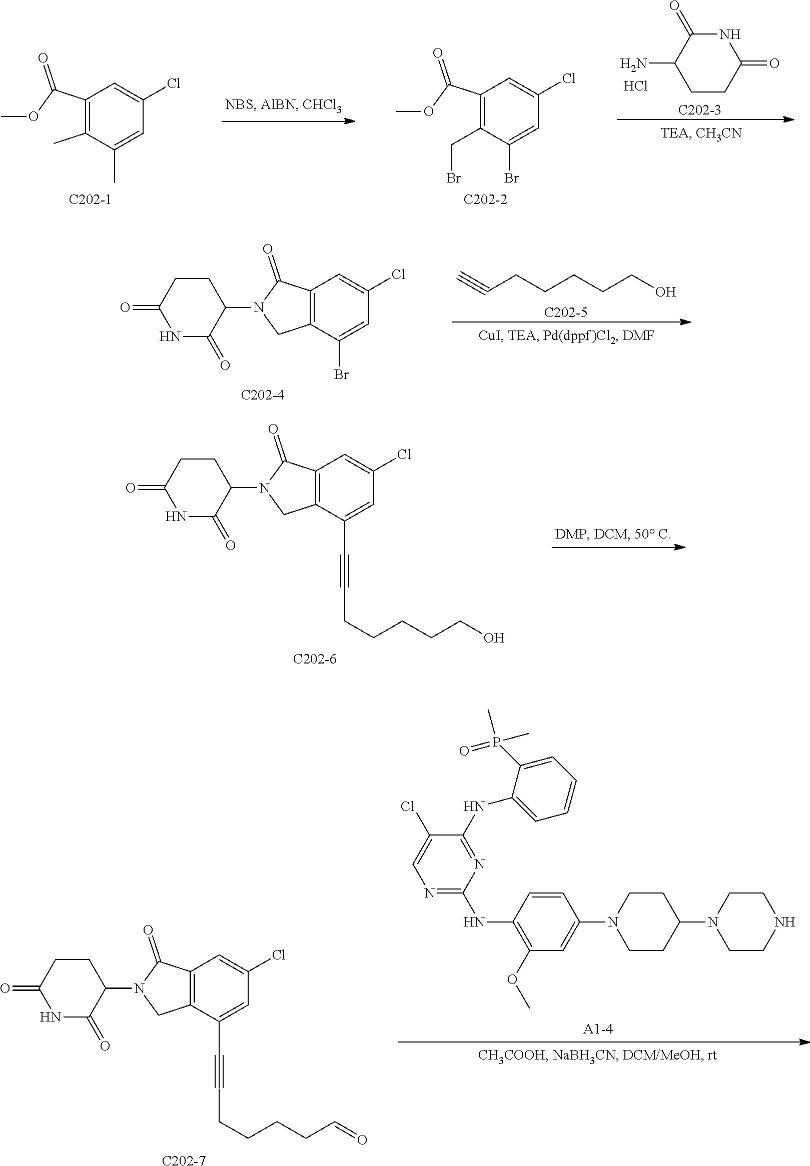

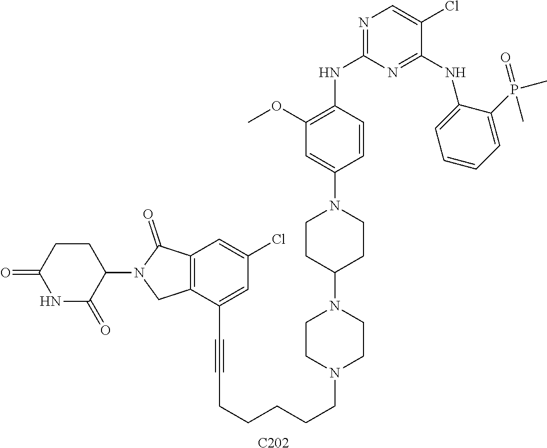 [SMILES: COc1cc(N2CCC(N3CCN(CCCCCC#Cc4cc(Cl)cc5c4CN(C4CCC(=O)NC4=O)C5=O)CC3)CC2)ccc1Nc1ncc(Cl)c(Nc2ccccc2P(C)(C)=O)n1 ; "C202"]

### Step 1

Compound C202-1 (1 g, 3.82 mmol), NBS (679 mg, 4.35 mmol), and AIBN (62 mg, 0.38 mmol) were dissolved in 20 mL of chloroform. The mixture was reacted under reflux at 95° C. under nitrogen protection for 5 h. The solvent was removed under reduced pressure, and the crude product was purified by Flash chromatography to afford white solid compound C202-2 (600 mg, yield: 46.2%).

### Step 2

Compound C202-2 (1.31 g, 3.85 mmol), compound C202-3 (822 mg, 5.01 mmol), and triethylamine (506 mg, 5.01 mmol) were dissolved in 10 mL of acetonitrile. The mixture was stirred at 80° C. under nitrogen protection overnight. The mixture was cooled to room temperature. The solvent was removed under reduced pressure, and the crude product was purified by Flash chromatography to afford white solid compound C202-4 (1.0 g, yield: 73.0%). LCMS: [M+H]⁺=357, 359.

### Step 3

Compound C202-4 (100 mg, 0.281 mmol), compound C202-5 (79 mg, 0.702 mmol), cuprous iodide (5.4 mg, 0.028 mmol), Pd(dppf)Cl₂ (21 mg, 0.028 mmol) and triethylamine (85 mg, 0.843 mmol) were dissolved in 2 mL of anhydrous DMF. The mixture was reacted with stirring at 70° C. under nitrogen protection for 5 h. The solvent was removed under reduced pressure, and the crude product was separated by preparative thin layer chromatography to afford off-white solid compound C202-6 (94 mg, yield: 87.9%). LCMS: [M+H]⁺=389.

### Step 4

Compound C202-6 (50 mg, 0.129 mmol) and Dess-Martin reagent (82 mg, 0.193 mmol) were dissolved in 10 mL of dichloromethane and stirred at 50° C. for 2 h. The mixture was cooled to room temperature, and quenched with saturated sodium bicarbonate aqueous solution. The organic phase was separated, and the aqueous phase was extracted with dichloromethane for three times. The organic phases were combined, dried with anhydrous sodium sulfate, and filtered. The filtrate was concentrated, and the crude product was purified by TLC to afford a white solid compound C202-7 (34 mg, yield: 68.0%). LCMS: [M+H]⁺=387.

### Step 5

Compound C202-7 (34 mg, 0.0881 mmol) and compound A1-4 (35 mg, 0.062 mmol) were dissolved in 1.5 mL of dichloromethane/methanol (2/1, v/v), and 1 drop of glacial acetic acid was added dropwise. The mixture was stirred at room temperature under nitrogen protection for 1 h. Then sodium cyanobohydride (3.9 mg, 0.062 mmol) was added. The mixture was further stirred for 1 h. The solvent was removed under reduced pressure below 40° C., and the crude product was purified by Flash chromatography to afford off-white solid compound C202 (10 mg, yield: 12.1%). LCMS: [M+H]⁺=940. ¹H NMR (400 MHz, DMSO-d₆) δ 11.18 (s, 1H), 11.03 (s, 1H), 8.48 (s, 1H), 8.06 (s, 2H), 7.72 (dd, 2H), 7.57-7.49 (m, 1H), 7.35 (dd, 2H), 7.09 (t, 1H), 6.62 (d, 1H), 6.47 (dd, 1H), 5.32 (m, 1H), 5.15 (dd, 1H), 4.46 (d, 1H), 4.31 (d, 1H), 3.76 (s, 3H), 3.71 (d, 2H), 2.97-2.87 (m, 1H), 2.65 (t, 3H), 2.59 (d, 3H), 2.34-2.32 (m, 4H), 2.27 (m, 3H), 2.03-1.97 (m, 2H), 1.82 (d, 2H), 1.76 (d, 7H), 1.59 (t, 2H), 1.48 (d, 7H).

### Preparation of 7-(7-(4-(1-(4-((5-chloro-4-((2-(dimethylphosphoryl)phenyl)amino)pyrimidin-2-yl)amino)-3-methoxyphenyl)piperidin-4-yl)piperazin-1-yl)hept-1-yn-1-yl)-2-(2,6-dioxopiperidin-3-yl)-3-oxoisoindoline-5-carbonitrile (C204)

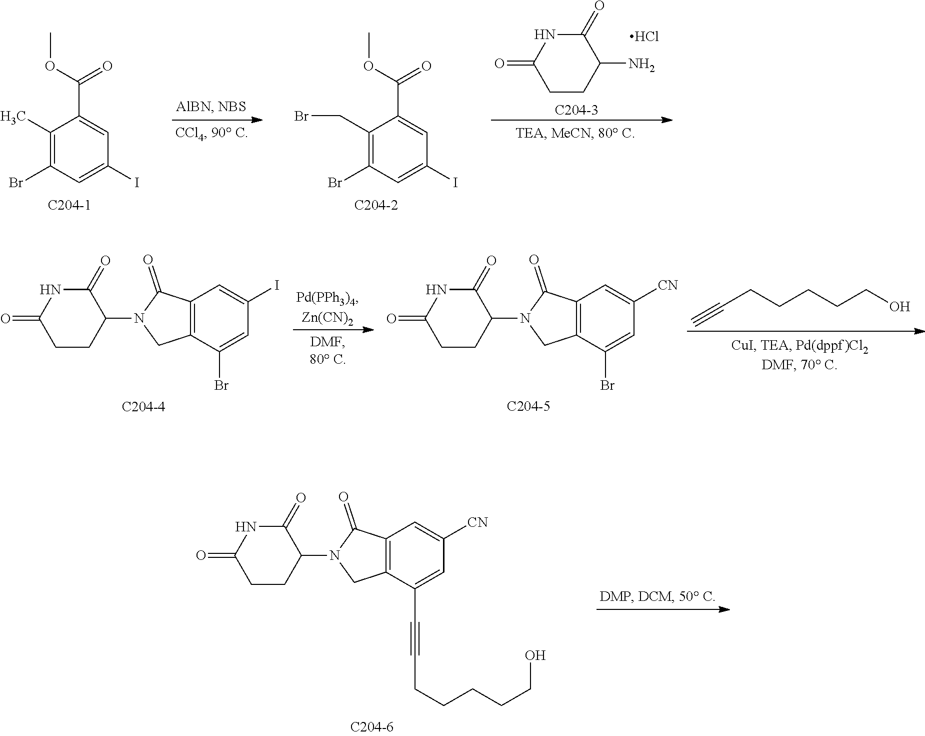

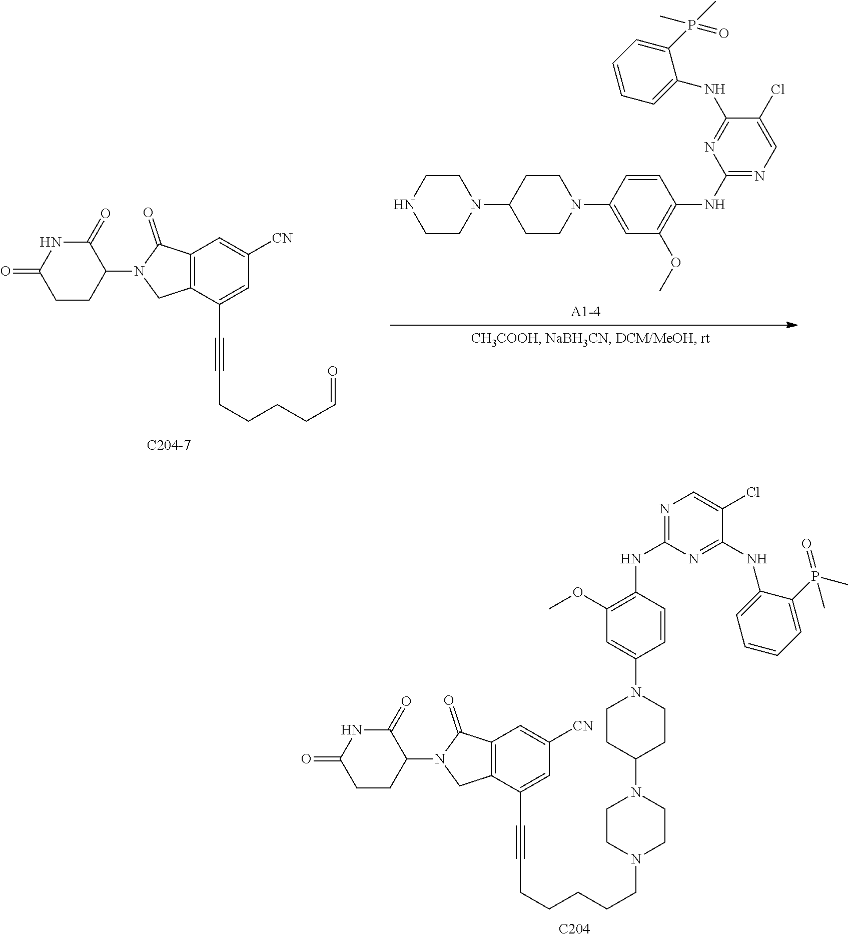 [SMILES: N#Cc1cc(C#CCCCCC=O)c2c(c1)C(=O)N(C1CCC(=O)NC1=O)C2 ; COc1cc(N2CCC(N3CCNCC3)CC2)ccc1Nc1ncc(Cl)c(Nc2ccccc2P(C)(C)=O)n1 ; COc1cc(N2CCC(N3CCN(CCCCCC#Cc4cc(C#N)cc5c4CN(C4CCC(=O)NC4=O)C5=O)CC3)CC2)ccc1Nc1ncc(Cl)c(Nc2ccccc2P(C)(C)=O)n1 ; "C204"]

### Step 1

C204-1 (532.5 mg, 1.5 mmol) was dissolved in 10.0 mL of CCl₄. NBS (351 mg, 1.95 mmol) and AIBN (73.8 mg, 0.45 mmol) were added under nitrogen protection. The mixture was heated to 90° C., and reacted under reflux for 20 h. TLC showed that one third of the raw materials were left. NBS (405 mg, 2.55 mmol) and AIBN (123 mg, 0.75 mmol) were added to the mixture. After the addition was completed, the mixture was further reacted under reflux for 20 h. TLC showed that there was no raw material remaining. The mixture was cooled to room temperature, and then filtered with suction. The filtrate was concentrated under reduced pressure to afford a crude product, which was purified by Flash chromatography to afford a light-yellow oily product, which became solid upon standing, C204-2 (690 mg, 100%).

### Step 2

C204-2 (690 mg, 1.6 mmol) and C204-3 (341 mg, 2.08 mmol) were dispersed in 12.0 mL of anhydrous MeCN, and TEA (210 mg, 2.08 mmol) was added. The mixture was heated to 80° C., and reacted under reflux for 16 h. LCMS showed that the reaction was completed. The mixture was cooled to room temperature, and filtrated with suction. The filter cake was washed with MeCN for three times, and dried with baking to afford a title product C204-4 (450 mg, yield: 62.8%). LCMS: [M+H]⁺=449, 451.

### Step 3

C204-4 (380 mg, 0.848 mmol) and Pd(PPh₃)₄ (98 mg, 0.0848 mmol) were dispersed in 20.0 mL of anhydrous DMF. Zn(CN)₂ (109.5 mg, 0.933 mmol) was added under N₂ protection. The mixture was heated to 80° C., and reacted under reflux for 2 h. LCMS showed that there was raw material remaining. Zn(CN)₂ (149.3 mg, 1.272 mmol) and Pd(PPh₃)₄ (284 mg, 0.2544 mmol) were added to the mixture. After the addition was completed, the mixture was further reacted at 80° C. for 18 h. LCMS showed that the reaction was completed. The mixture was cooled to room temperature and directly purified by RP-Flash chromatography to afford brown solid product C204-5 (170 mg, 57.8%). LCMS: [M+H]⁺=348, 350.

### Step 4

C204-5 (160 mg, 0.461 mmol), CuI (17.5 mg, 0.092 mmol) and Pd(dppf)Cl₂ (67.4 mg, 0.092 mmol) were dispersed in 20.0 mL of anhydrous DMF. 6-alkynyl-1-heptanol (129.1 mg, 1.153 mmol) and TEA (140 mg, 1.383 mmol) were added successively under N₂ protection. The mixture was heated to 70° C. and reacted for 20 h. LCMS showed that the reaction was completed. The mixture was cooled to room temperature and directly purified by RP-Flash chromatography to afford pale brown solid product C204-6 (140 mg, 91.4%). LCMS: [M+H]⁺=380.

### Step 5

C204-6 (140 mg, 0.369 mmol) was dissolved in 28.0 mL of anhydrous DCM. Dess-Martin reagent (313 mg, 0.738 mmol) was added under N₂ protection. The mixture was heated to 50° C., and reacted under reflux for 2.0 h. TLC showed that the reaction was completed. The mixture was cooled to room temperature, and then 20 mL of saturated NaHCO₃ solution and 20 mL of saturated Na₂S₂O₃ solution were added. The mixture was stirred at room temperature for 5 min. The organic layer was separated, dried with anhydrous sodium sulfate, and filtered with suction. The filtrate was concentrated under reduced pressure to afford a crude product. The crude product was purified by Prep-TLC to afford light-yellow solid product C204-7 (80 mg, yield: 57.1%). LCMS: [M+H]⁺=378.

### Step 6

C204-7 (45 mg, 0.119 mmol) and A1-4 (61 mg, 0.107 mmol) were dissolved in a mixture of 4.0 mL of anhydrous DCM and 0.4 mL of anhydrous MeOH. CH₃COOH (10.7 mg, 0.179 mmol) was added under nitrogen protection. The mixture was stirred at room temperature for 0.5 h. Then solid NaBH₃CN (15.0 mg, 0.238 mmol) was added. The mixture was further reacted at room temperature for 2 h. LCMS and TLC showed that the reaction was completed. The mixture was purified by Prep-HPLC to afford white solid pure product C204 (35 mg, yield: 31.8%). LCMS: [M+H]⁺=931.

### Preparation of 3-(4-((7-(9-(4-((5-bromo-4-((5-(dimethylphosphoryl)quinoxalin-6-yl)amino)pyrimidin-2-yl)amino)-5-methoxy-2-methylphenyl)-3,9-diazaspiro[5.5]undecan-3-yl)heptyl)oxy)-1-oxoisoindolin-2-yl)piperidine-2,6-dione (C211)

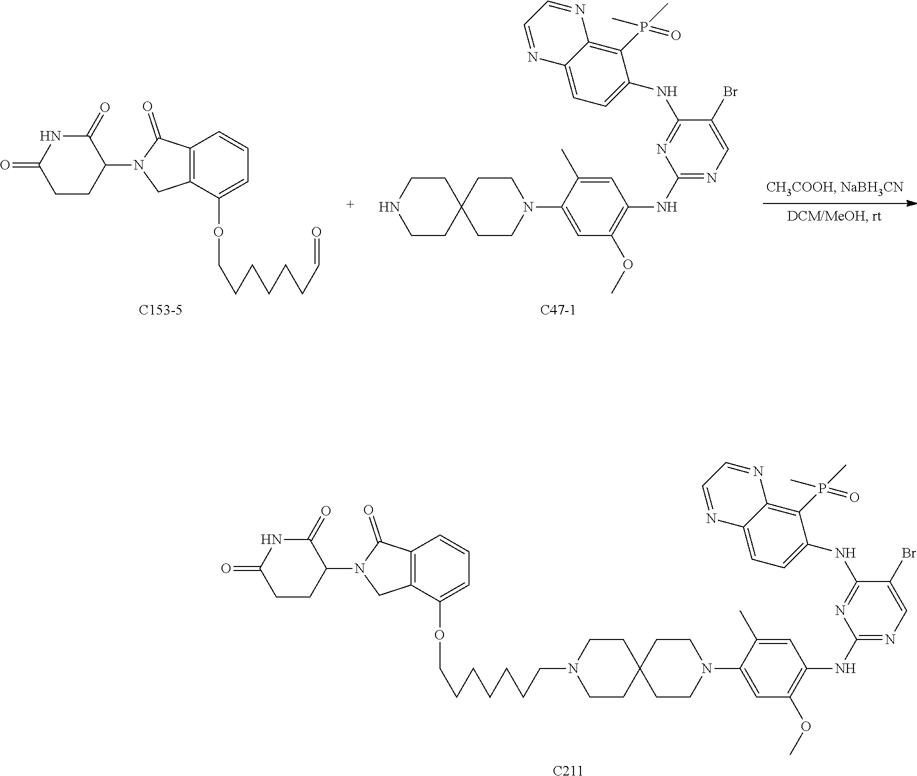

C153-5 (35 mg, 0.094 mmol) and C47-1 (56.2 mg, 0.0846 mmol) were dissolved in a mixture of 4.0 mL of anhydrous DCM and 0.4 mL of anhydrous MeOH. CH₃COOH (8.46 mg, 0.1410 mmol) was added dropwise under nitrogen protection. The mixture was stirred at room temperature for 30 min. Then NaBH₃CN (8.8 mg, 0.1410 mmol) was added. The mixture was reacted with stirring at room temperature for 2 h. After the reaction was completed, the mixture was purified by Prep-HPLC to afford a yellow solid pure product C211 (25 mg, yield: 26.1%). LCMS: [M+H]⁺=1021, 1023.

### Preparation of 3-(4-((6-(9-(4-((5-bromo-4-((5-(dimethylphosphoryl)quinoxalin-6-yl)amino)pyrimidin-2-yl)amino)-5-methoxy-2-methylphenyl)-3,9-diazaspiro[5.5]undecan-3-yl)hexyl)oxy)-1-oxoisoindolin-2-yl)piperidine-2,6-dione (C212)

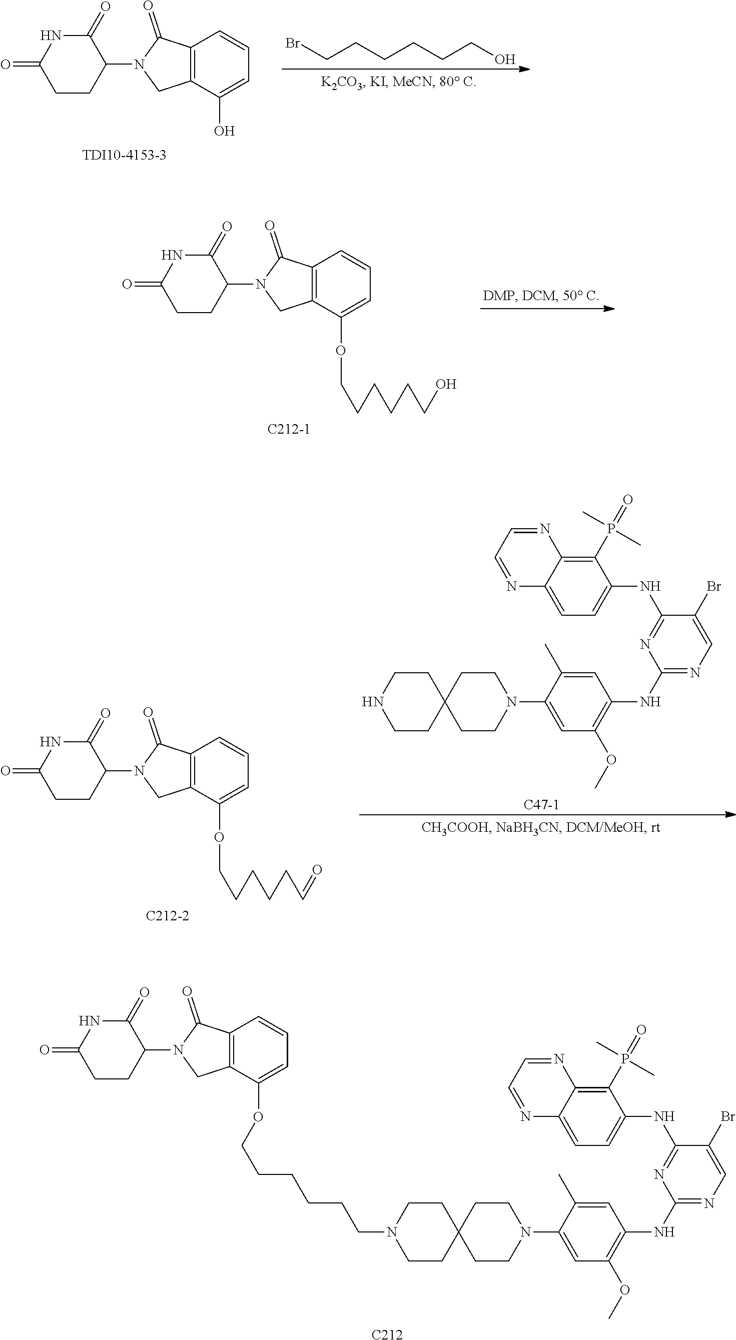

### Step 1

C153-3 (100 mg, 0.3846 mmol), K₂CO₃ (79.6 mg, 0.5769 mmol) and KI (31.9 mg, 0.1923 mmol) were dispersed in 15.0 mL of anhydrous MeCN, and 6-bromohexan-1-ol (125 mg, 0.6923 mmol) was added. The mixture was reacted at 80° C. for 3.0 h. The mixture was cooled to room temperature, and filtered with suction to remove solid residues. The filtrate was concentrated under reduced pressure to afford a crude product, which was purified by Prep-TLC to afford light-yellow solid product C212-1 (40 mg, 32.0%). LCMS: [M+H]⁺=361.

### Step 2

C212-1 (40 mg, 0.111 mmol) was dissolved in 15.0 mL of anhydrous DCM, and Dess-Martin reagent (94.1 mg, 0.222 mmol) was added. The mixture was reacted under reflux at 50° C. for 2 h. The mixture was cooled to room temperature, and diluted with 10 mL of DCM. 8.0 mL of saturated NaHCO₃ solution and 8.0 mL of saturated Na₂S₂O₃ solution were added. The mixture was stirred for 5 min. The organic layer was separated, dried with anhydrous Na₂SO₄, and filtered with suction to remove the desiccant. The filtrate was concentrated under reduced pressure to afford a crude product, which was purified by Prep-TLC to afford light-yellow oily product C212-2 (20 mg, yield: 50.4%). LCMS: [M+H]⁺=359.

### Step 3

C212-2 (20 mg, 0.056 mmol) and C47-1 (31.5 mg, 0.048 mmol) were dissolved in a mixture of 4.0 mL of anhydrous DCM and 0.4 mL of anhydrous MeOH. CH₃COOH (5.03 mg, 0.084 mmol, dissolved in 0.5 mL of DCM) was added dropwise under nitrogen protection. The mixture was stirred at room temperature for 30 min. Then NaBH₃CN (7.02 mg, 0.112 mmol) was added. The mixture was reacted with stirring at room temperature for 2 h. The mixture was directly purified by Prep-TLC to afford a crude product, which was further purified by Prep-TLC to afford yellow solid pure product C212 (20 mg, yield: 35.7%). LCMS: [M+H]⁺=1007, 1009.

### Preparation of 3-(4-((5-(9-(4-((5-bromo-4-((5-(dimethylphosphoryl)quinoxalin-6-yl)amino)pyrimidin-2-yl)amino)-5-methoxy-2-methylphenyl)-3,9-diazaspiro[5.5]undecan-3-yl)pentyl)oxy)-1-oxoisoindolin-2-yl)piperidine-2,6-dione (C213)

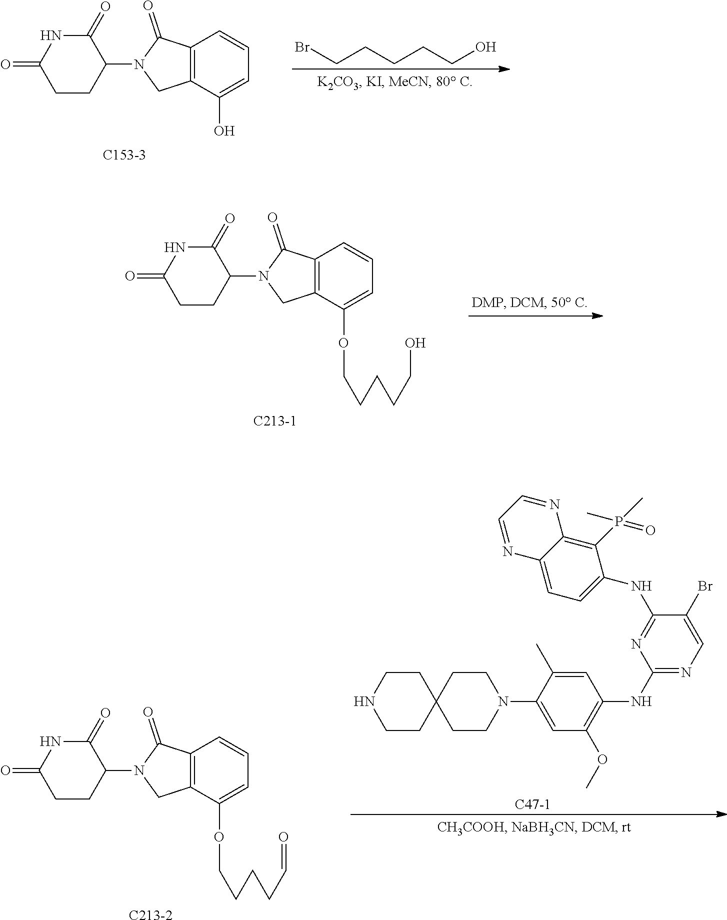

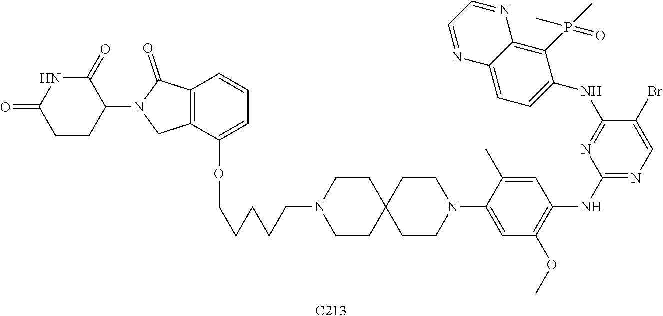 [SMILES: COc1cc(N2CCC3(CCN(CCCCCOc4cccc5c4CN(C4CCC(=O)NC4=O)C5=O)CC3)CC2)c(C)cc1Nc1ncc(Br)c(Nc2ccc3nccnc3c2P(C)(C)=O)n1 ; "C213"]

### Step 1

C153-3 (130 mg, 0.5 mmol), K₂CO₃ (103.5 mg, 0.75 mmol) and KI (41.5 mg, 0.25 mmol) were dispersed in 15.0 mL of anhydrous MeCN, and 5-bromopentan-1-ol (125.3 mg, 0.75 mmol) was added. The mixture was reacted at 80° C. for 3.0 h. The mixture was cooled to room temperature, and then filtered with suction to remove solid residues. The filtrate was concentrated under reduced pressure to afford a crude product, which was purified by Prep-TLC to afford light-yellow solid product C213-1 (65 mg, 37.6%). LCMS: [M+H]⁺=347.

### Step 2

C213-1 (65 mg, 0.188 mmol) was dissolved in 15.0 mL of anhydrous DCM, and Dess-Martin reagent (159.4 mg, 0.376 mmol) was added. The mixture was reacted under reflux at 50° C. for 2 h. The mixture was cooled to room temperature, and diluted with 20 mL of DCM. 10.0 mL of saturated NaHCO₃ solution and 10.0 mL of saturated Na₂S₂O₃ solution were added. The mixture was stirred for 5 min. The organic layer was separated, dried with anhydrous Na₂SO₄, and filtered with suction to remove the desiccant. The filtrate was concentrated under reduced pressure to afford crude C213-2 (57 mg, 87.7%). The crude product was directly used in the next step. LCMS: [M+H]⁺=345.

### Step 3

C213-2 (34.4 mg, 0.1 mmol) and C47-1 (39.8 mg, 0.06 mmol) were dissolved in 4.0 mL of anhydrous DCM. CH₃COOH (9.0 mg, 0.15 mmol, dissolved in 0.5 mL of DCM) was added dropwise under nitrogen protection. The mixture was stirred at room temperature for 30 min. Then NaBH₃CN (12.6 mg, 0.2 mmol) was added. The mixture was reacted with stirring at room temperature for 2 h. The mixture was purified by Prep-HPLC to afford yellow solid pure product C213 (13.0 mg, yield: 13.1%). LCMS: [M+H]⁺=993, 995.

### Preparation of 3-(4-((7-(4-(1-(4-((5-bromo-4-((5-(dimethylphosphoryl)quinoxalin-6-yl)amino)pyrimidin-2-yl)amino)-5-methoxy-2-methylphenyl)piperidin-4-yl)piperazin-1-yl)heptyl)oxy)-1-oxoisoindolin-2-yl)piperidine-2,6-dione (C214)

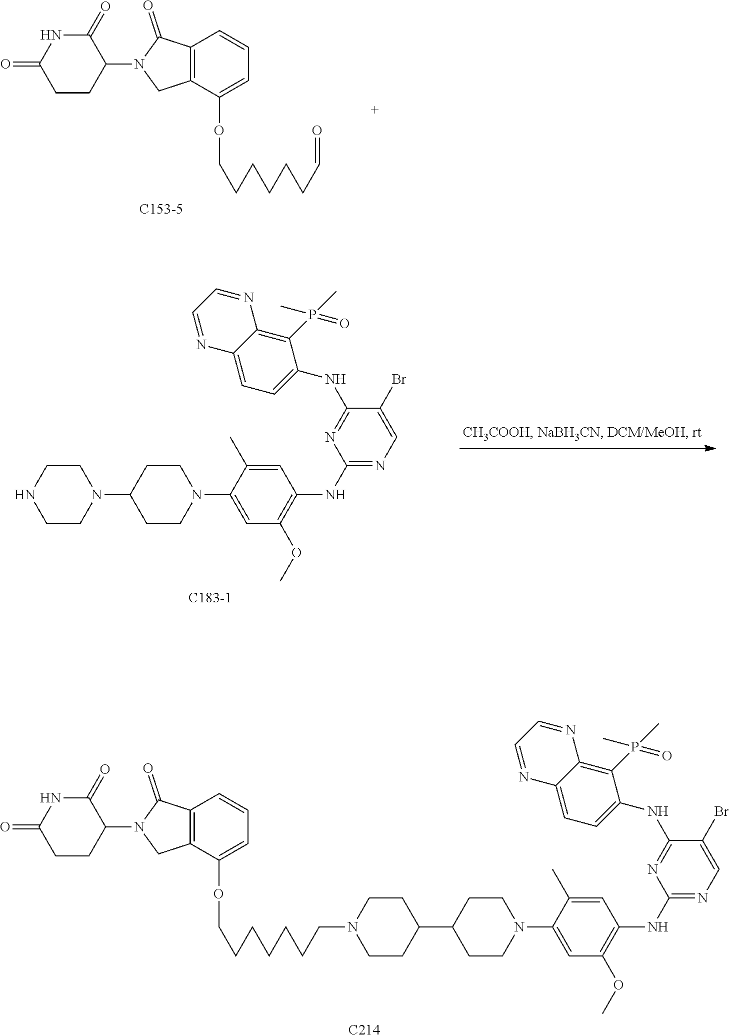

C153-5 (30 mg, 0.081 mmol) and C183-1 (or C158-1) (49.3 mg, 0.073 mmol) were dissolved in a mixture of 4.0 mL of anhydrous DCM and 0.4 mL of anhydrous MeOH. CH₃COOH (7.29 mg, 0.1215 mmol) was added dropwise under nitrogen protection. The mixture was stirred at room temperature for 30 min. Then NaBH₃CN (7.6 mg, 0.1215 mmol) was added. The mixture was reacted with stirring at room temperature for 2 h. The mixture was purified by Prep-HPLC to afford yellow solid pure product C214 (25 mg, yield: 29.9%). LCMS: [M+H]⁺=1036, 1038.

### Preparation of 3-(4-(4-(4-(1-(4-((5-chloro-4-((2-(dimethylphosphoryl)phenyl)amino)pyrimidin-2-yl)amino)-3-methoxyphenyl)piperidin-4-yl)piperazin-1-yl)but-1-yn-1-yl)-1-oxoisoindolin-2-yl)piperidine-2,6-dione (C215)

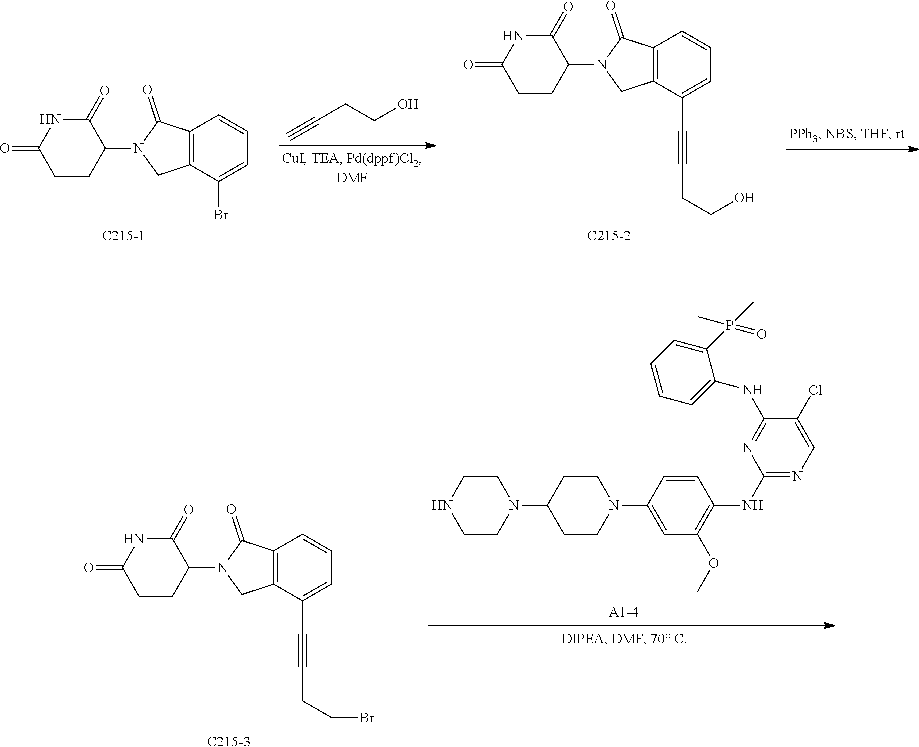

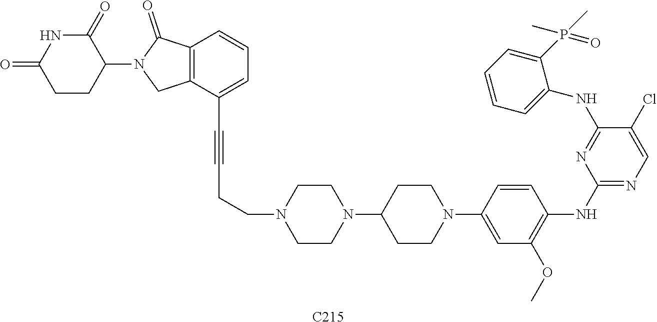 [SMILES: COc1cc(N2CCC(N3CCN(CCC#Cc4cccc5c4CN(C4CCC(=O)NC4=O)C5=O)CC3)CC2)ccc1Nc1ncc(Cl)c(Nc2ccccc2P(C)(C)=O)n1 ; "C215"]

### Step 1

C215-1 (or C1-1) (322 mg, 1.0 mmol), CuI (19.0 mg, 0.1 mmol) and Pd(dppf)Cl₂ (73.1 mg, 0.1 mmol) were dispersed in 10.0 mL of anhydrous DMF. 3-alkynyl-1-butanol (140.2 mg, 2.0 mmol) and TEA (303 mg, 3.0 mmol) were added successively under N₂ protection. The mixture was heated to 70° C. and reacted for 20 h. LCMS showed that the reaction was completed. The mixture was cooled to room temperature and directly purified by RP-Flash chromatography to afford light-yellow solid product C215-2 (210 mg, 67.3%). LCMS: [M+H]⁺=313.

### Step 2

C215-2 (112 mg, 0.359 mmol) and PPh₃ (103.5 mg, 0.395 mmol) were dissolved in 33.0 mL of anhydrous THF. NBS (127.8 mg, 0.718 mmol) was added under N2 protection. The mixture was reacted at room temperature for 5.0 h. The mixture was concentrated under reduced pressure to afford a crude product, which was purified by Prep-TLC to afford white solid product C215-3 (105 mg, 78.3%). LCMS: [M+H]⁺=375, 377.

### Step 3

C215-3 (45 mg, 0.12 mmol) and A1-4 (54.8 mg, 0.096 mmol) were dissolved in 4.5 mL of anhydrous DMF. DIPEA (75.8 mg, 0.6 mmol) was added under nitrogen protection. The mixture was reacted at 70° C. for 12 h, and then purified by RP-Flash to afford white solid product C215 (12 mg, yield: 9.2%). LCMS: [M+H]⁺=864.

### Preparation of 3-(5-(5-(4-(1-(4-((5-chloro-4-((2-(dimethylphosphoryl)phenyl)amino)pyrimidin-2-yl)amino)-3-methoxyphenyl)piperidin-4-yl)piperazin-1-yl)pent-1-yn-1-yl)-1-oxoisoindolin-2-yl)piperidine-2,6-dione (C218)

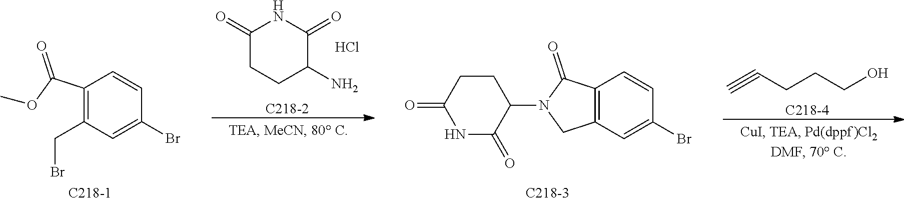

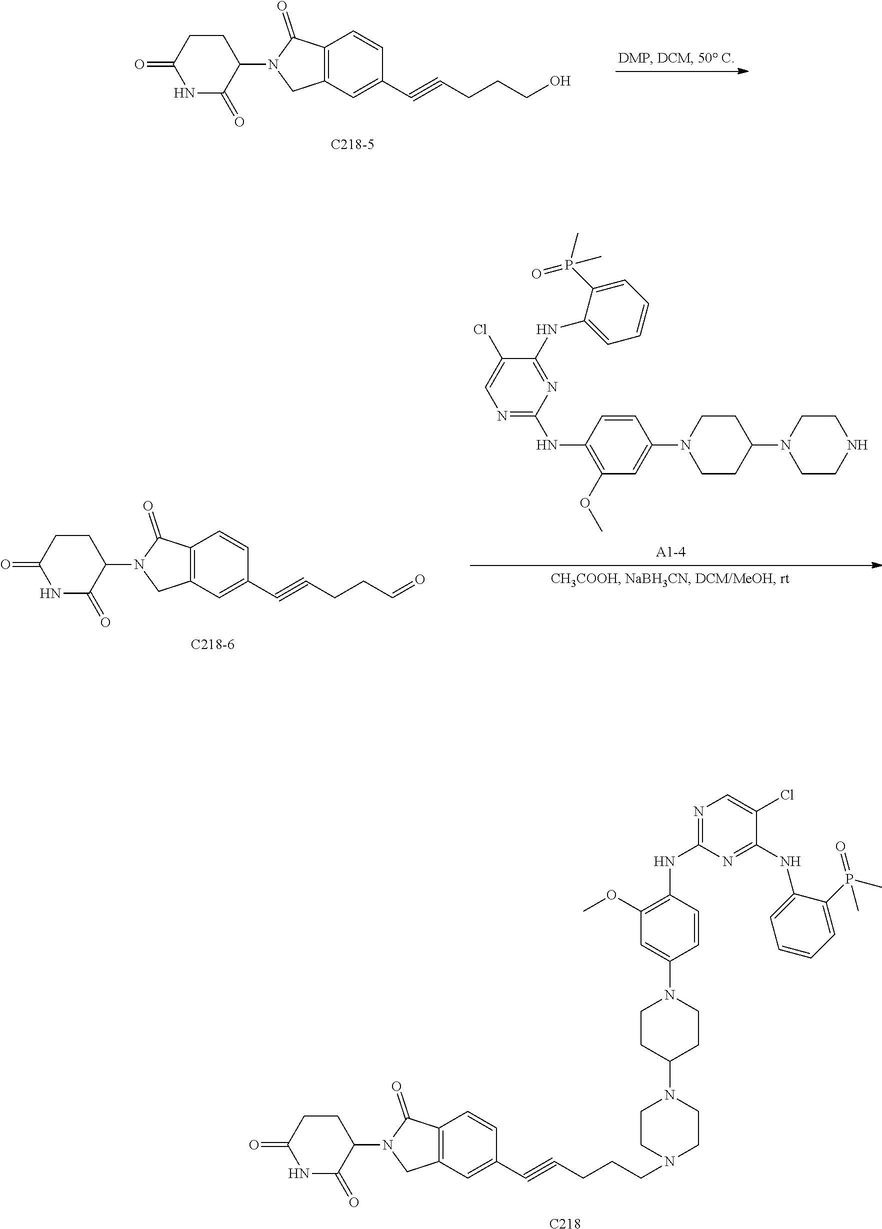

### Step 1

Compound C218-1 (300 mg, 0.974 mmol), C218-2 (208 mg, 1.266 mmol), and triethylamine (128 mg, 1.266 mmol) were dissolved in 5 mL of acetonitrile. The mixture was stirred at 80° C. under nitrogen protection overnight. The mixture was cooled to room temperature. The solvent was removed under reduced pressure, and the crude product was purified by Flash chromatography to afford white solid compound C218-3 (180 mg, yield: 57.0%). LCMS: [M+H]⁺=323, 325.

### Step 2

Compound C218-3 (90 mg, 0.281 mmol), compound C218-4 (59 mg, 0.699 mmol), cuprous iodide (5.3 mg, 0.028 mmol), Pd(dppf)Cl₂ (20.5 mg, 0.028 mmol) and triethylamine (85 mg, 0.839 mmol) were dissolved in 3 mL of anhydrous DMF. The mixture was reacted with stirring at 70° C. under nitrogen protection for 5 h. The solvent was removed under reduced pressure, and the crude product was purified by thin layer chromatography to afford off-white solid compound C218-5 (83 mg, yield: 91.2%). LCMS: [M+H]⁺=327.

### Step 3

Compound C218-5 (50 mg, 0.153 mmol) and Dess-Martin reagent (98 mg, 0.230 mmol) were dissolved in 10 mL of dichloromethane and stirred at 50° C. for 2 h. The mixture was cooled to room temperature, and quenched with saturated sodium bicarbonate aqueous solution. The organic phase was separated, and the aqueous phase was extracted with dichloromethane for three times. The organic phases were combined, dried with anhydrous sodium sulfate, and concentrated. The crude product was purified by TLC to afford a white solid compound C218-6 (38 mg, yield: 76.0%). LCMS: [M+H]⁺=325.

### Step 4

Compound C218-6 (38 mg, 0.119 mmol) and compound A1-4 (40 mg, 0.095 mmol) were dissolved in 3 mL of dichloromethane/methanol (2/1, v/v), and 1 drop of glacial acetic acid was added dropwise. The mixture was stirred at room temperature under nitrogen protection for 1 h. Then sodium cyanobohydride (6.0 mg, 0.095 mmol) was added. The mixture was further stirred for 1 h. The solvent was removed under reduced pressure below 40° C., and the crude product was purified by Flash chromatography to afford off-white solid compound C218 (25 mg, yield: 24.3%). LCMS: [M+H]⁺=878. ¹H NMR (400 MHz, DMSO-d₆) δ 11.17 (s, 1H), 10.99 (s, 1H), 8.48 (dr, 1H), 8.06 (d, 2H), 7.69 (dd, 1H), 7.63 (t, 1H), 7.56-7.49 (m, 2H), 7.35 (dd, 2H), 7.13-7.06 (m, 1H), 6.63 (d, 1H), 6.47 (dd, 1H), 5.32 (m, 1H), 5.11 (dd, 1H), 4.48-4.28 (m, 2H), 3.76 (s, 3H), 3.72 (d, 2H), 2.91 (m, 1H), 2.72-2.60 (m, 4H), 2.57 (m, 2H), 2.40 (m, 7H), 2.03-1.98 (m, 3H), 1.86 (d, 2H), 1.78 (s, 3H), 1.74 (s, 3H), 1.72 m, 2H), 1.59-1.50 (m, 2H), 1.50-1.43 (m, 1H).

### Preparation of 3-(4-(5-(4-(1-(4-((5-bromo-4-((5-(dimethylphosphoryl)quinoxalin-6-yl)amino)pyrimidin-2-yl)amino)-5-methoxy-2-methylphenyl)piperidin-4-yl)piperazin-1-yl)pent-1-yn-1-yl)-1-oxoisoindolin-2-yl)piperidine-2,6-dione (C227)

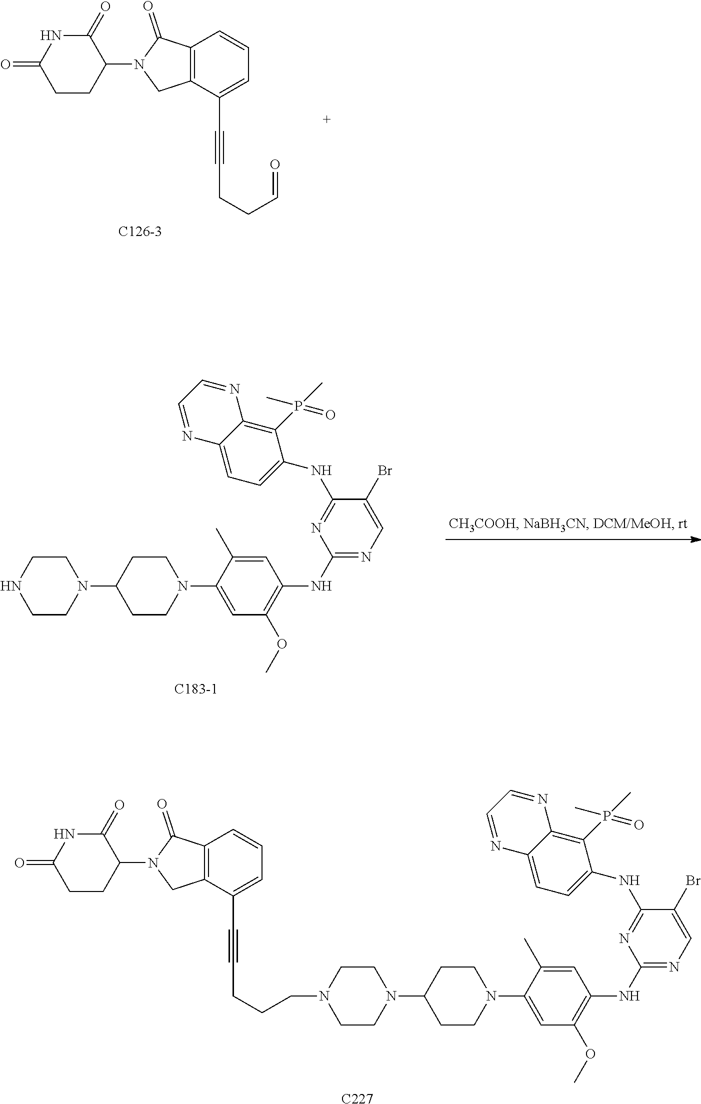

C126-3 (32.4 mg, 0.1 mmol) and C183-1 (or C158-1) (61.1 mg, 0.09 mmol) were dissolved in a mixture of 4.0 mL of anhydrous DCM and 0.4 mL of anhydrous MeOH. CH₃COOH (9.0 mg, 0.15 mmol) was added under nitrogen protection. The mixture was stirred at room temperature for 0.5 h. Then solid NaBH₃CN (12.6 mg, 0.2 mmol) was added. The mixture was further reacted at room temperature for 2 h. LCMS and TLC showed that the reaction was completed. The mixture was purified by Prep-HPLC to afford yellow solid pure product C227 (20 mg, yield: 20.3%). LCMS: [M+H]⁺=988, 990.

### Preparation of 3-(4-(5-(9-(4-((5-bromo-4-((5-(dimethylphosphoryl)quinoxalin-6-yl)amino)pyrimidin-2-yl)amino)-5-methoxy-2-methylphenyl)-3,9-diazaspiro[5.5]undecan-3-yl)pent-1-yn-1-yl)-1-oxoisoindolin-2-yl)piperidine-2,6-dione (C228)

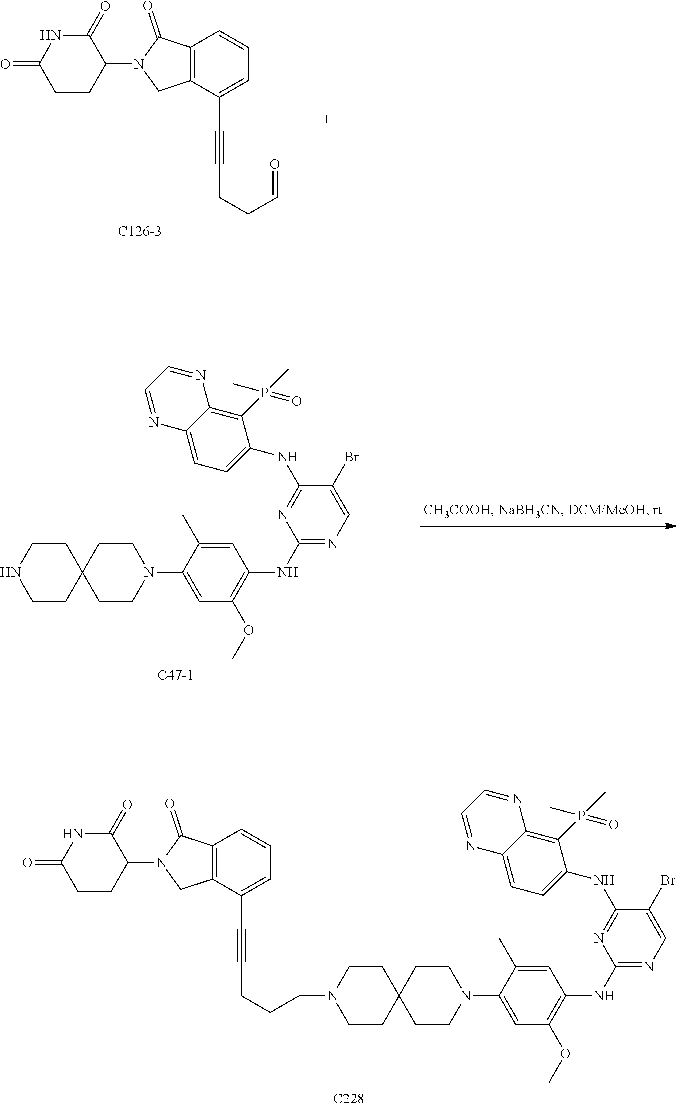

C126-3 (32.4 mg, 0.1 mmol) and C47-1 (59.8 mg, 0.09 mmol) were dissolved in a mixture of 4.0 mL of anhydrous DCM and 0.4 mL of anhydrous MeOH. CH₃COOH (9.0 mg, 0.15 mmol) was added under nitrogen protection. The mixture was stirred at room temperature for 0.5 h. Then solid NaBH₃CN (12.6 mg, 0.2 mmol) was added. The mixture was further reacted at room temperature for 2 h. LCMS and TLC showed that the reaction was completed. The mixture was purified by Prep-HPLC to afford yellow solid pure product C228 (30 mg, yield: 30.9%). LCMS: [M+H]⁺=973, 975.

### Preparation of 7-(7-(4-(1-(4-((5-bromo-4-((5-(dimethylphosphoryl)quinoxalin-6-yl)amino)pyrimidin-2-yl)amino)-5-methoxy-2-methylphenyl)piperidin-4-yl)piperazin-1-yl)hept-1-yn-1-yl)-2-(2,6-dioxopiperidin-3-yl)-3-oxoisoindoline-5-carbonitrile (C229)

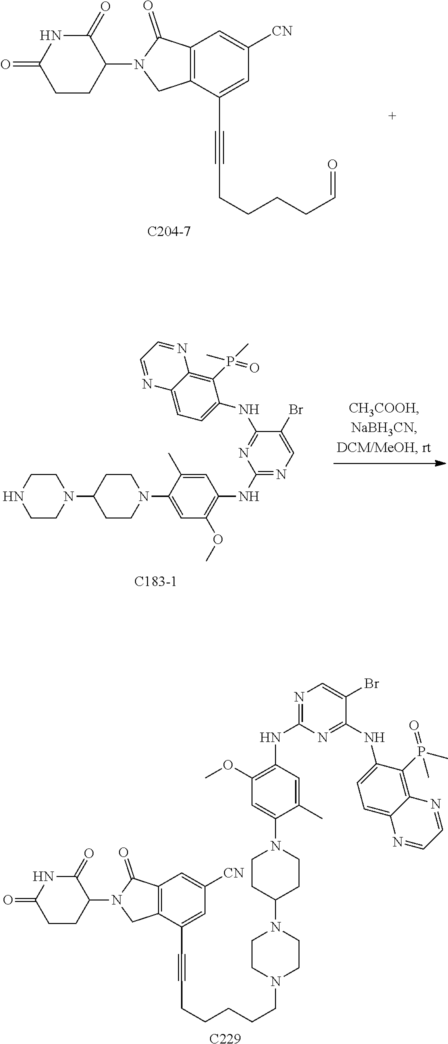

C204-7 (35 mg, 0.093 mmol) and C183-1 (or C158-1) (56.7 mg, 0.0837 mmol) were dissolved in a mixture of 4.0 mL of anhydrous DCM and 0.4 mL of anhydrous MeOH. CH₃COOH (8.36 mg, 0.1395 mmol) was added under nitrogen protection. The mixture was stirred at room temperature for 0.5 h. Then solid NaBH₃CN (11.7 mg, 0.1860 mmol) was added. The mixture was further reacted at room temperature for 2 h. LCMS and TLC showed that the reaction was completed. The mixture was purified by Prep-HPLC to afford yellow solid pure product C229 (25 mg, yield: 25.8%). LCMS: [M+H]⁺=1041, 1043.

### Preparation of 3-(5-(4-(3-(4-(1-(4-((5-chloro-4-((2-(dimethylphosphoryl)phenyl)amino)pyrimidin-2-yl)amino)-3-methoxyphenyl)piperidin-4-yl)piperazin-1-yl)propyl)phenyl)-1-oxoisoindolin-2-yl)piperidine-2,6-dione (C235)

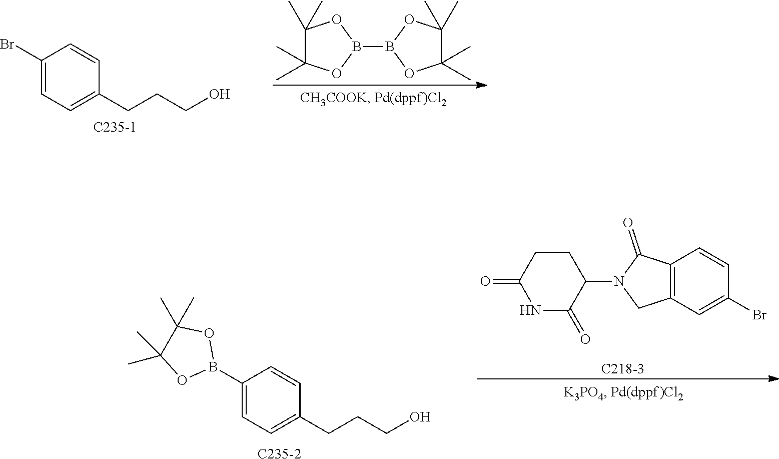

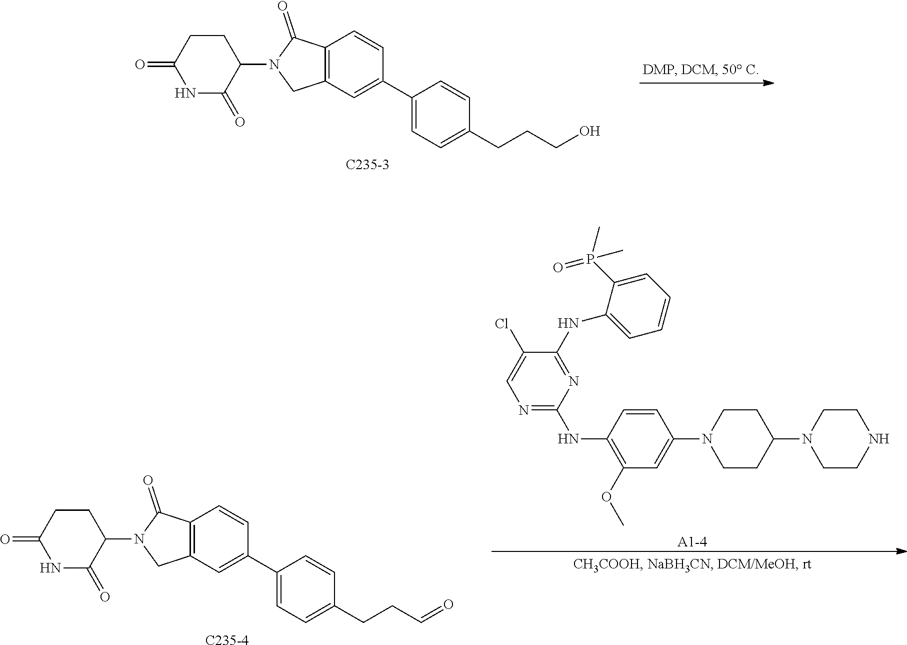 [SMILES: "C235-4" ; "C235-3" ; O=CCCc1ccc(-c2ccc3c(c2)CN(C2CCC(=O)NC2=O)C3=O)cc1 ; O=C1CCC(N2Cc3cc(-c4ccc(CCCO)cc4)ccc3C2=O)C(=O)N1 ; COc1cc(N2CCC(N3CCNCC3)CC2)ccc1Nc1ncc(Cl)c(Nc2ccccc2P(C)(C)=O)n1 ; "A1-4"]

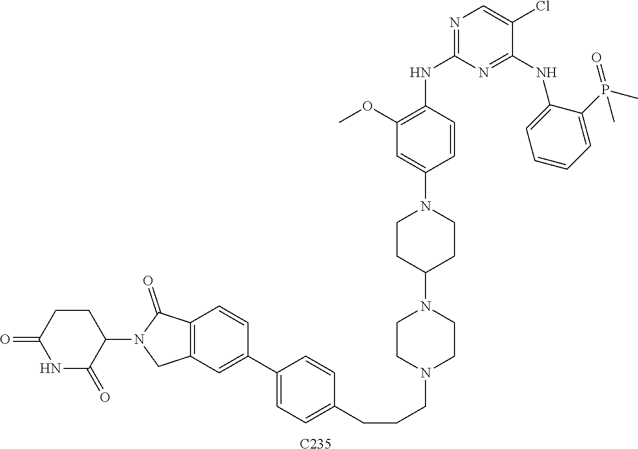 [SMILES: COc1cc(N2CCC(N3CCN(CCCc4ccc(-c5ccc6c(c5)CN(C5CCC(=O)NC5=O)C6=O)cc4)CC3)CC2)ccc1Nc1ncc(Cl)c(Nc2ccccc2P(C)(C)=O)n1 ; "C235"]

### Step 1

Compound C235-1 (600 mg, 2.84 mmol), bis(pinacolato)diboron (805 mg, 3.17 mmol), potassium acetate (825 mg, 8.41 mmol), and Pd(dppf)Cl₂ (62 mg, 0.08 mmol) were dissolved in 10 mL of 1,4-dioxane. The mixture was stirred at 100° C. under nitrogen protection overnight. The solvent was removed under reduced pressure, and the crude product was purified by Flash chromatography to afford compound C235-2 (550 mg, yield: 74.9%).

### Step 2

C218-3 (or C1-1) (80 mg, 0.248 mmol), compound C235-2 (85 mg, 0.323 mmol), potassium phosphate (65 mg, 0.298 mmol), and Pd(dppf)Cl₂ (18 mg, 0.025 mmol) were dissolved in 1.5 mL of DMF. The mixture was stirred at 90° C. under nitrogen protection overnight. The solvent was removed under reduced pressure, and the crude product was purified by Flash chromatography to afford compound C235-3 (52 mg, yield: 45.2%). LCMS: [M+H]⁺=379.

### Step 3

Compound C235-3 (78 mg, 0.206 mmol) and Dess-Martin reagent (175 mg, 0.413 mmol) were dissolved in 10 mL of dichloromethane and stirred at 50° C. for 2 h. The mixture was cooled to room temperature, and saturated sodium bicarbonate aqueous solution and saturated sodium thiosulfate aqueous solution were added successively and stirred for 5 minutes respectively to quench the reaction. The organic phase was separated, and the aqueous phase was extracted with dichloromethane for three times. The organic phases were combined, dried with anhydrous sodium sulfate, and filtered. The filtrate was concentrated, and the crude product was purified by TLC to afford a white solid compound C235-4 (44 mg, yield: 57.1%). LCMS: [M+H]⁺=377.

### Step 4

Compound C235-4 (22 mg, 0.058 mmol) and compound A1-4 (27 mg, 0.047 mmol) were dissolved in 2 mL of dichloromethane/methanol (2/1, v/v), and glacial acetic acid (3.5 mg, 0.058 mmol) was added dropwise. The mixture was stirred at room temperature under nitrogen protection for 1 h. Then sodium cyanobohydride (2.9 mg, 0.047 mmol) was added. The mixture was further stirred for 1 h. The solvent was removed under reduced pressure below 40° C., and the crude product was purified by Flash chromatography to afford off-white solid compound C235 (12 mg, yield: 22.2%). LCMS: [M+H]⁺=930.

### Preparation of 3-(5-(4-(4-(4-(1-(4-((5-chloro-4-((2-(dimethylphosphoryl)phenyl)amino)pyrimidin-2-yl)amino)-3-methoxyphenyl)piperidin-4-yl)piperazin-1-yl)butyl)phenyl)-1-oxoisoindolin-2-yl)piperidine-2,6-dione (C236)

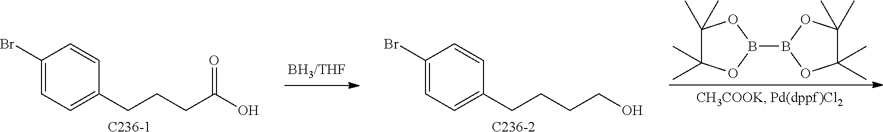

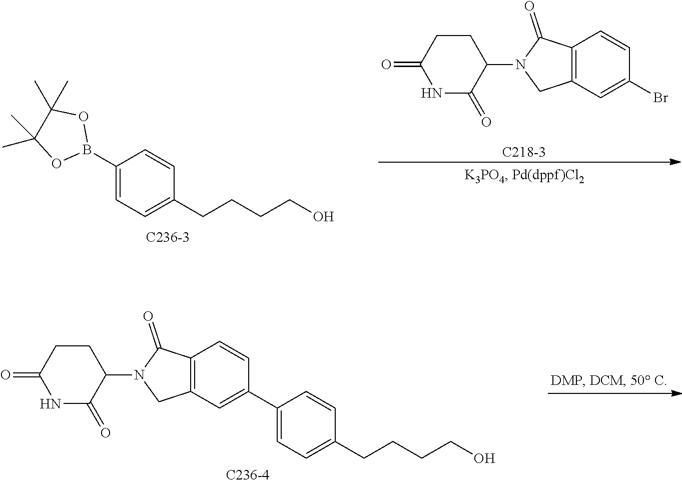 [SMILES: "C236-3" ; "C236-4" ; "C218-3" ; CC1(C)OB(c2ccc(CCCCO)cc2)OC1(C)C ; O=C1CCC(N2Cc3cc(-c4ccc(CCCCO)cc4)ccc3C2=O)C(=O)N1 ; O=C1CCC(N2Cc3cc(Br)ccc3C2=O)C(=O)N1]

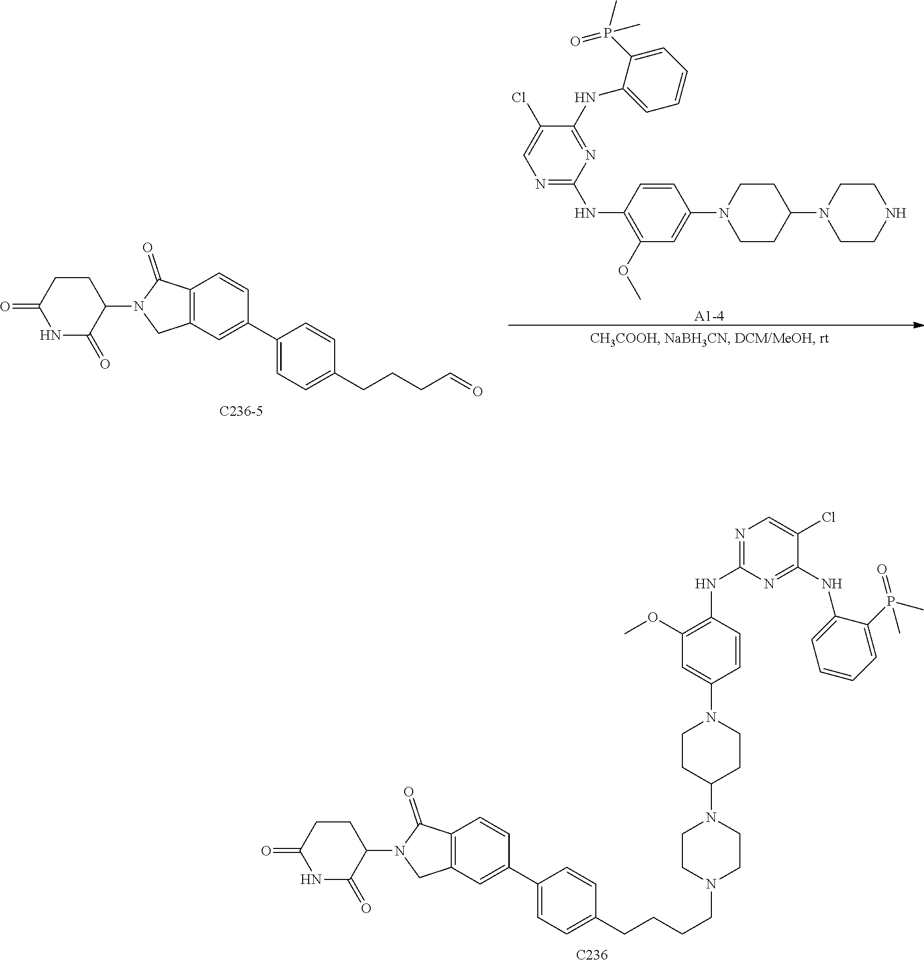 [SMILES: "C236" ; "C236-5" ; O=CCCCc1ccc(-c2ccc3c(c2)CN(C2CCC(=O)NC2=O)C3=O)cc1 ; COc1cc(N2CCC(N3CCN(CCCCc4ccc(-c5ccc6c(c5)CN(C5CCC(=O)NC5=O)C6=O)cc4)CC3)CC2)ccc1Nc1ncc(Cl)c(Nc2ccccc2P(C)(C)=O)n1 ; COc1cc(N2CCC(N3CCNCC3)CC2)ccc1Nc1ncc(Cl)c(Nc2ccccc2P(C)(C)=O)n1 ; "A1-4"]

### Step 1

Compound C236-1 (1 g, 4.13 mmol) was dissolved in 10 mL of dry THF. The mixture was cooled to −10° C., and the tetrahydrofuran complex of borane (1 mol/L, 8.3 mL, 8.26 mmol) was added dropwise under nitrogen protection. After the dropwise addition, the mixture was warmed to room temperature and stirred for 2 h. The mixture was poured into 10 mL of cold water. The mixture was extracted with ethyl acetate, dried with anhydrous sodium sulfate, and filtered. The filtrate was concentrated. The crude product was purified by Flash chromatography to afford compound C236-2 (600 mg, yield: 63.7%).

### Step 2

Compound C236-2 (635 mg, 2.78 mmol), bis(pinacolato)diboron (800 mg, 3.15 mmol), potassium acetate (820 mg, 8.35 mmol), and Pd(dppf)Cl₂ (61 mg, 0.08 mmol) were dissolved in 10 mL of 1,4-dioxane. The mixture was stirred at 100° C. under nitrogen protection overnight. The solvent was removed under reduced pressure, and the crude product was purified by Flash chromatography to afford compound C236-3 (500 mg, yield: 65.1%).

### Step 3

C218-3 (or C1-1) (100 mg, 0.311 mmol), compound C236-3 (112 mg, 0.404 mmol), potassium phosphate (81 mg, 0.373 mmol), and Pd(dppf)Cl₂ (23 mg, 0.031 mmol) were dissolved in 2 mL of DMF. The mixture was stirred at 90° C. under nitrogen protection overnight. The solvent was removed under reduced pressure, and the crude product was purified by Flash chromatography to afford compound C236-4 (60 mg, yield: 49.6%). LCMS: [M+H]⁺=393.

### Step 4

Compound C236-4 (42 mg, 0.107 mmol) and Dess-Martin reagent (91 mg, 0.214 mmol) were dissolved in 10 mL of dichloromethane and stirred at 50° C. for 2 h. The mixture was cooled to room temperature, and saturated sodium bicarbonate aqueous solution and saturated sodium thiosulfate aqueous solution were added successively and stirred for 5 minutes respectively to quench the reaction. The organic phase was separated, and the aqueous phase was extracted with dichloromethane for three times. The organic phases were combined, dried with anhydrous sodium sulfate, and filtered. The filtrate was concentrated, and the crude product was purified by TLC to afford a white solid compound C236-5 (39 mg, yield: 93.3%). LCMS: [M+H]⁺=391.

### Step 5

Compound C236-5 (39 mg, 0.1 mmol) and compound A1-4 (40 mg, 0.07 mmol) were dissolved in 2 mL of dichloromethane/methanol (2/1, v/v), and glacial acetic acid (6.0 mg, 0.1 mmol) was added dropwise. The mixture was stirred at room temperature under nitrogen protection for 1 h. Then sodium cyanobohydride (4.4 mg, 0.07 mmol) was added. The mixture was further stirred for 1 h. The solvent was removed under reduced pressure below 40° C., and the crude product was purified by Flash chromatography to afford off-white solid compound C236 (2.6 mg, yield: 2.8%). LCMS: [M+H]⁺=944.

### Preparation of 3-(4-(6-(4-(1-(4-((5-chloro-4-((2-(dimethylphosphoryl)phenyl)amino)pyrimidin-2-yl)amino)-3-methoxyphenyl)piperidin-4-yl)piperazin-1-yl)hexyl)-1-oxoisoindolin-2-yl)piperidine-2,6-dione (C238)

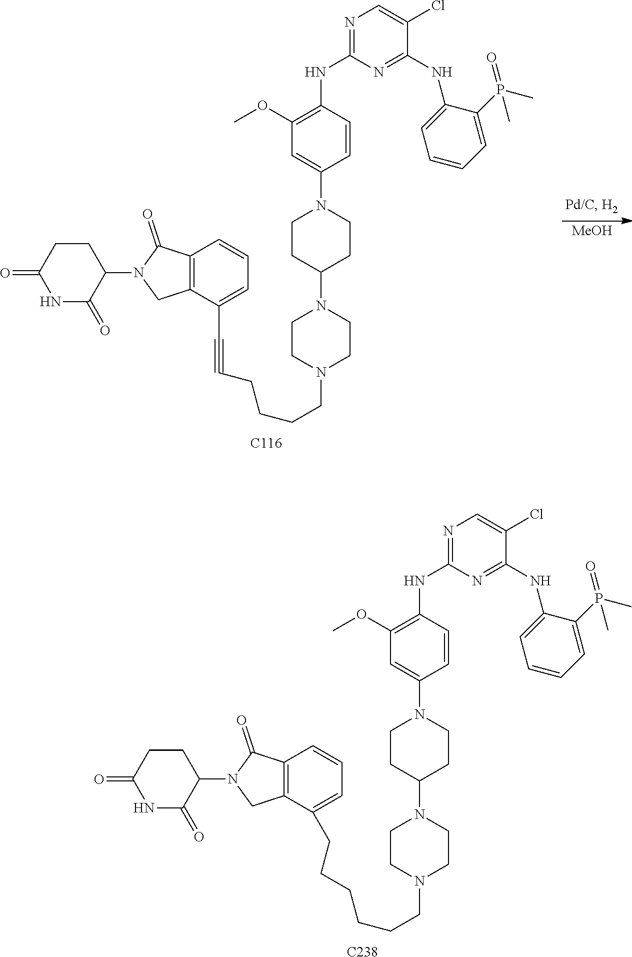 [SMILES: "MeOH" ; COc1cc(N2CCC(N3CCN(CCCCC#Cc4cccc5c4CN(C4CCC(=O)NC4=O)C5=O)CC3)CC2)ccc1Nc1ncc(Cl)c(Nc2ccccc2P(C)(C)=O)n1 ; "C116" ; COc1cc(N2CCC(N3CCN(CCCCCCc4cccc5c4CN(C4CCC(=O)NC4=O)C5=O)CC3)CC2)ccc1Nc1ncc(Cl)c(Nc2ccccc2P(C)(C)=O)n1 ; "C238"]

C116 (100 mg, 0.112 mmol) was dissolved in methanol (10 mL), and Pd/C (30 mg, 10% Pd) was added. The atmosphere was replaced three times with nitrogen gas, and then replaced three times with hydrogen gas. The atmosphere was pressurized to 60 Psi with hydrogen gas. The mixture was reacted with stirring at room temperature for 1 h. The reaction process was monitored by LCMS, and the raw materials were basically transformed into the title product. The atmosphere was replaced with nitrogen gas to remove hydrogen gas. The mixture was filtered to remove Pd/C. The filtrate was concentrated to afford a crude product, which was separated by Flash to afford white solid C238 (49 mg, yield: 48.8%). LCMS: [M+H]⁺=896.

### Preparation of 3-(4-(8-(4-(1-(4-((5-chloro-4-((2-(dimethylphosphoryl)phenyl)amino)pyrimidin-2-yl)amino)-3-methoxyphenyl)piperidin-4-yl)piperazin-1-yl)octyl)-1-oxoisoindolin-2-yl)piperidine-2,6-dione (C240)

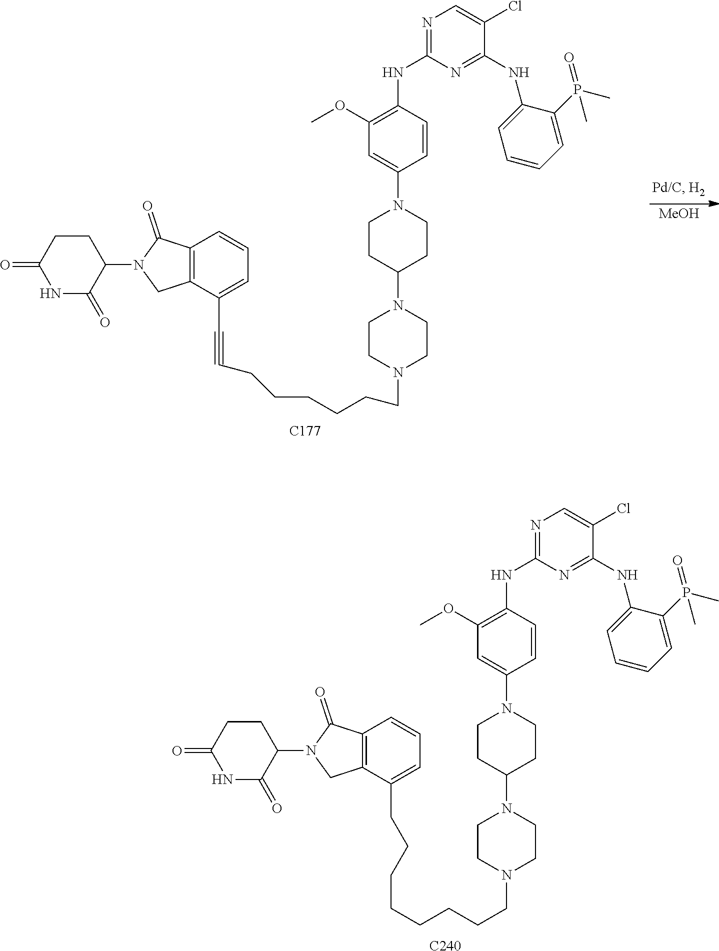 [SMILES: "MeOH" ; COc1cc(N2CCC(N3CCN(CCCCCCC#Cc4cccc5c4CN(C4CCC(=O)NC4=O)C5=O)CC3)CC2)ccc1Nc1ncc(Cl)c(Nc2ccccc2P(C)(C)=O)n1 ; "C177" ; COc1cc(N2CCC(N3CCN(CCCCCCCCc4cccc5c4CN(C4CCC(=O)NC4=O)C5=O)CC3)CC2)ccc1Nc1ncc(Cl)c(Nc2ccccc2P(C)(C)=O)n1 ; "C240"]

C177 (50 mg, 0.051 mmol) was dissolved in methanol (10 mL), and Pd/C (20 mg, 10% Pd) was added. The atmosphere was replaced three times with nitrogen gas, and then replaced three times with hydrogen gas. The atmosphere was pressurized to 60 Psi with hydrogen gas. The mixture was reacted with stirring at room temperature for 1 h. The reaction process was monitored by LCMS, and the raw materials were basically transformed into the title product. The atmosphere was replaced with nitrogen gas to remove hydrogen gas. The mixture was filtered to remove Pd/C. The filtrate was concentrated to afford a crude product, which was separated by Flash to afford white solid C240, 19 mg, yield: 40.4%. LCMS: [M+H]⁺=924.

### Preparation of 3-(4-(7-(4-(1-(4-((5-chloro-4-((2-(dimethylphosphoryl)phenyl)amino)pyrimidin-2-yl)amino)-3-methoxyphenyl)piperidin-4-yl)piperazin-1-yl)hept-1-yn-1-yl)-7-fluoro-1-oxoisoindolin-2-yl)piperidine-2,6-dione (C243)

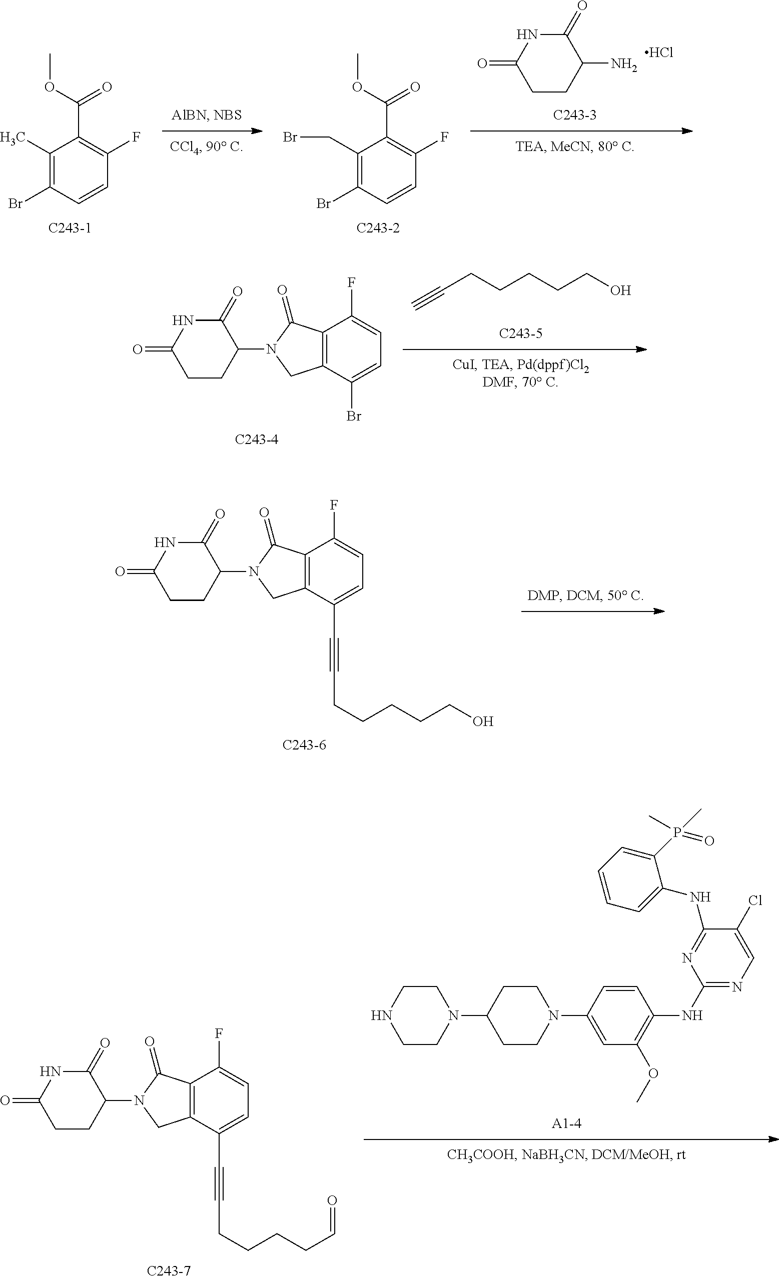

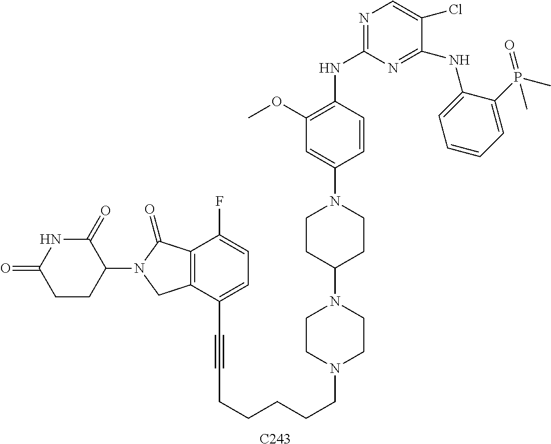 [SMILES: COc1cc(N2CCC(N3CCN(CCCCCC#Cc4ccc(F)c5c4CN(C4CCC(=O)NC4=O)C5=O)CC3)CC2)ccc1Nc1ncc(Cl)c(Nc2ccccc2P(C)(C)=O)n1 ; "C243"]

### Step 1

C243-1 (1.0 g, 4.05 mmol) was dissolved in 20.0 mL of CCl₄. NBS (1.09 g, 6.08 mmol) and AIBN (265.7 mg, 1.62 mmol) were added under nitrogen protection. The mixture was heated to 90° C., and reacted under reflux for 20 h. TLC showed that one fifth of the raw materials were left. NBS (1.09 g, 6.08 mmol) and AIBN (265.7 mg, 1.62 mmol) were added to the mixture. After the addition was completed, the mixture was further reacted under reflux for 20 h. TLC showed that there was no raw material remaining. The mixture was cooled to room temperature, and then filtered with suction. The filtrate was concentrated under reduced pressure to afford a crude product, which was purified by Flash chromatography to afford light-yellow oily product C243-2 (1.35 g, 100%).

### Step 2

C243-2 (1.35 g, 4.18 mmol) and C243-3 (891 mg, 5.43 mmol) were dispersed in 25.0 mL of anhydrous MeCN, and TEA (548.8 mg, 5.43 mmol) was added. The mixture was heated to 80° C., and reacted under reflux for 16 h. LCMS showed that the reaction was completed. The mixture was cooled to room temperature, and then filtrated with suction. The filter cake was washed with MeCN for three times, and dried with baking to afford a title product C243-4 (1.25 g, yield: 88.0%). LCMS: [M+H]⁺=341, 343.

### Step 3

C243-4 (200 mg, 0.588 mmol), CuI (22.3 mg, 0.118 mmol) and Pd(dppf)Cl₂ (86.2 mg, 0.118 mmol) were dispersed in 10.0 mL of anhydrous DMF. C243-5 (131.8 mg, 1.176 mmol) and TEA (178.2 mg, 1.764 mmol) were added successively under N₂ protection. The mixture was heated to 70° C. and reacted for 20 h. LCMS showed that the reaction was completed. The mixture was cooled to room temperature and directly purified by RP-Flash chromatography to afford light-yellow solid product C243-6 (160 mg, 73.1%). LCMS: [M+H]⁺=373.

### Step 4

C243-6 (160 mg, 0.43 mmol) was dissolved in 80.0 mL of anhydrous DCM. Dess-Martin reagent (365 mg, 0.86 mmol) was added under N₂ protection. The mixture was heated to 50° C., and reacted under reflux for 2.0 h. TLC showed that the reaction was completed. The mixture was cooled to room temperature, and then 20 mL of saturated NaHCO₃ solution and 20 mL of saturated Na₂S₂O₃ solution were added. The mixture was stirred at room temperature for 5 min. The organic layer was separated, dried with anhydrous sodium sulfate, and filtered with suction. The filtrate was concentrated under reduced pressure to afford a crude product. The crude product was purified by Flash chromatography to afford light-yellow solid product C243-7 (75 mg, 46.9%). LCMS: [M+H]⁺=371.

### Step 5

C243-7 (72 mg, 0.195 mmol) and A1-4 (99.9 mg, 0.176 mmol) were dissolved in a mixture of 7.0 mL of anhydrous DCM and 0.7 mL of anhydrous MeOH. CH₃COOH (17.6 mg, 0.293 mmol) was added under nitrogen protection. The mixture was stirred at room temperature for 0.5 h. Then solid NaBH₃CN (24.5 mg, 0.390 mmol) was added. The mixture was further reacted at room temperature for 2 h. LCMS and TLC showed that the reaction was completed. The mixture was purified by Prep-HPLC to afford white solid pure product C243 (45 mg, yield: 25.0%). LCMS: [M+H]⁺=924.

### Preparation of 3-(4-(6-(4-(1-(4-((5-chloro-4-((2-(dimethylphosphoryl)phenyl)amino)pyrimidin-2-yl)amino)-3-methoxyphenyl)piperidin-4-yl)piperazin-1-yl)hex-1-yn-1-yl)-7-fluoro-1-oxoisoindolin-2-yl)piperidine-2,6-dione (C244)

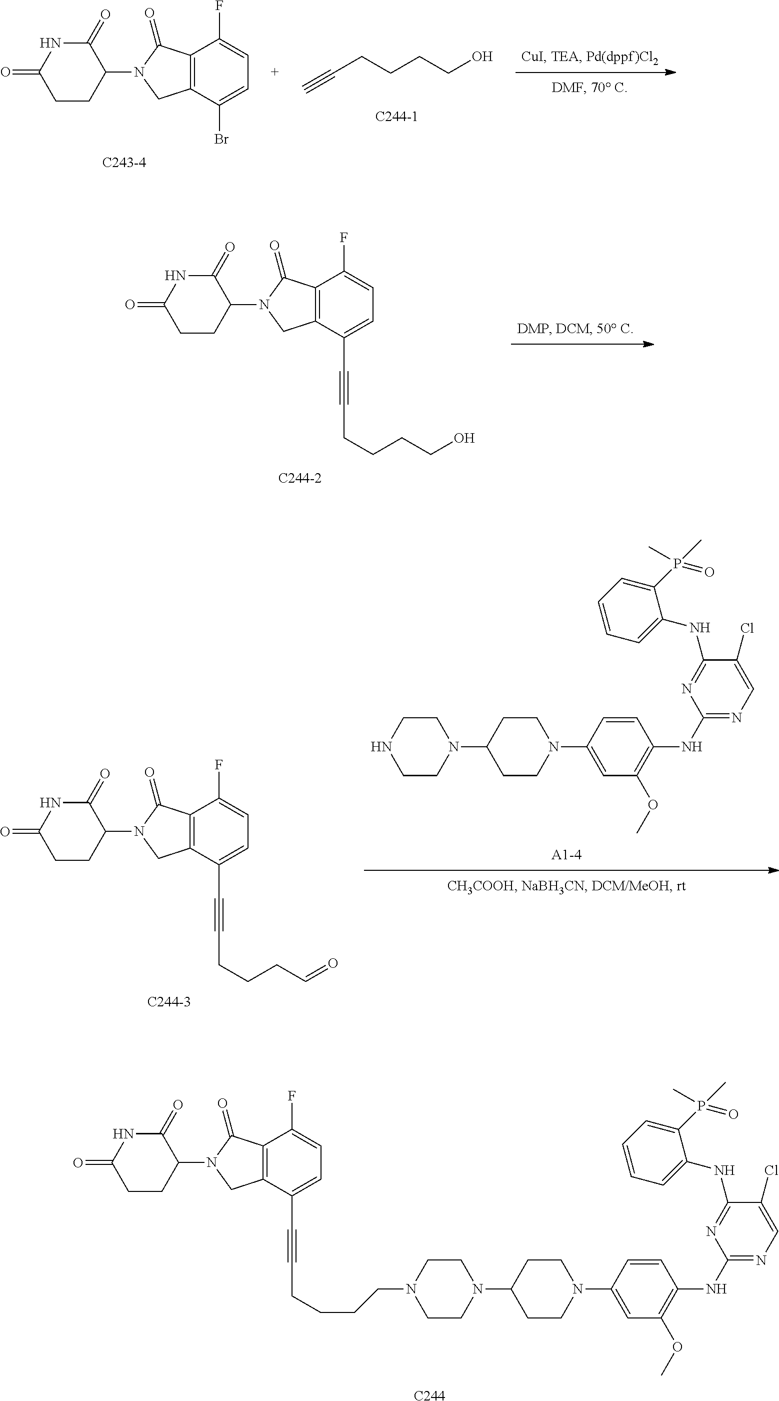

### Step 1

C243-4 (170 mg, 0.5 mmol), CuI (19.0 mg, 0.1 mmol) and Pd(dppf)Cl₂ (73.1 mg, 0.1 mmol) were dispersed in 8.5 mL of anhydrous DMF. C244-1 (122.5 mg, 1.25 mmol) and TEA (151.5 mg, 1.5 mmol) were added successively under N₂ protection. The mixture was heated to 70° C. and reacted for 20 h. LCMS showed that the reaction was completed. The mixture was cooled to room temperature and directly purified by RP-Flash chromatography to afford light-yellow solid crude C244-2 (210 mg, yield: 93.8%), and the crude product was directly used in the next step. LCMS: [M+H]⁺=359.

### Step 2

C244-2 (90 mg, 0.251 mmol) was dissolved in 45.0 mL of anhydrous DCM. Dess-Martin reagent (159.6 mg, 0.377 mmol) was added under N₂ protection. The mixture was heated to 50° C., and reacted under reflux for 2.0 h. TLC showed that the reaction was completed. The mixture was cooled to room temperature, and then 15 mL of saturated NaHCO₃ solution and 15 mL of saturated Na₂S₂O₃ solution were added. The mixture was stirred at room temperature for 5 min. The organic layer was separated, dried with anhydrous sodium sulfate, and filtered with suction. The filtrate was concentrated under reduced pressure to afford a crude product. The crude product was purified by Prep-TLC to afford light-yellow solid product C244-3 (75 mg, 83.3%). LCMS: [M+H]⁺=357.

### Step 3

C244-3 (71.2 mg, 0.2 mmol) and A1-4 (102.4 mg, 0.18 mmol) were dissolved in a mixture of 7.0 mL of anhydrous DCM and 0.7 mL of anhydrous MeOH. CH₃COOH (18.0 mg, 0.3 mmol) was added under nitrogen protection. The mixture was stirred at room temperature for 0.5 h. Then solid NaBH₃CN (25.1 mg, 0.4 mmol) was added. The mixture was further reacted at room temperature for 2 h. LCMS and TLC showed that the reaction was completed. The mixture was purified by Prep-HPLC to afford white solid pure product C244 (50 mg, yield: 27.5%). LCMS: [M+H]⁺=910.

### Preparation of 3-(4-(7-(4-(1-(4-((5-chloro-4-((2-(dimethylphosphoryl)phenyl)amino)pyrimidin-2-yl)amino)-3-methoxyphenyl)piperidin-4-yl)piperazin-1-yl)heptyl)-1-oxoisoindolin-2-yl)piperidine-2,6-dione (C245)

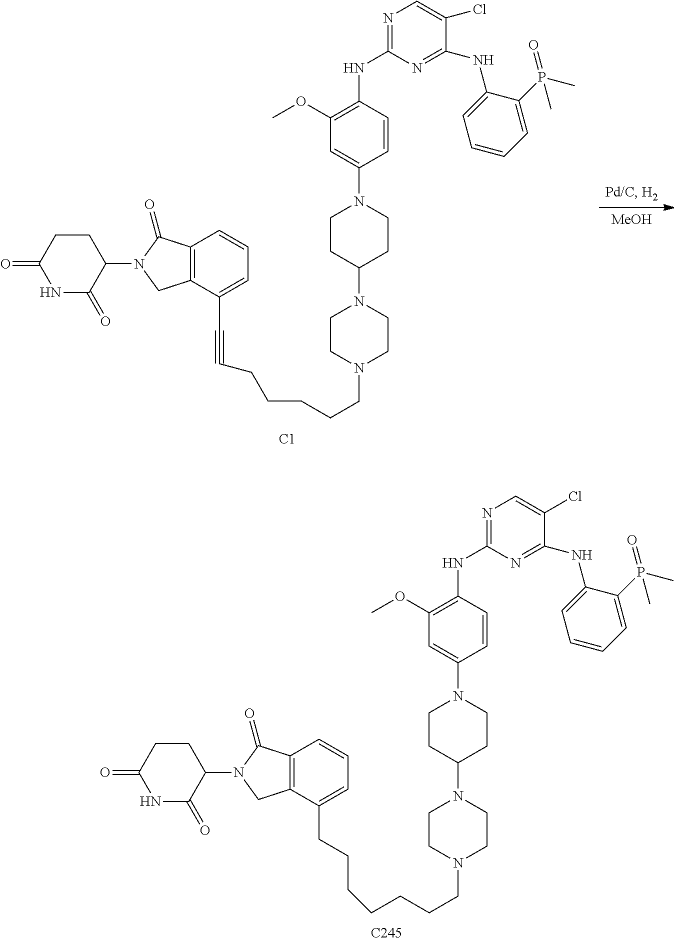

Compound C1 (45 mg, 0.05 mmol) was dissolved in methanol (10 mL), and Pd/C (20 mg, 10% Pd) was added. The atmosphere was replaced three times with nitrogen gas, and then replaced three times with hydrogen gas. The atmosphere was pressurized to 60 Psi with hydrogen gas. The mixture was reacted with stirring at room temperature for 1 h. The reaction process was monitored by LCMS until the raw materials were basically transformed into the title product. The atmosphere was replaced with nitrogen gas to remove hydrogen gas. The mixture was filtered to remove Pd/C. The filtrate was concentrated to afford a crude product, which was separated by Flash to afford 7 mg of white solid. Yield: 16%. LCMS: [M+H]⁺=876.

### Preparation of 3-(4-((5-(4-(1-(4-((5-bromo-4-((5-(dimethylphosphoryl) quinoxalin-6-yl)amino)pyrimidin-2-yl)amino)-5-methoxy-2-methylphenyl)piperidin-4-yl)piperazin-1-yl)pentyl)oxy)-1-oxoisoindolin-2-yl)piperidine-2,6-dione (C246)

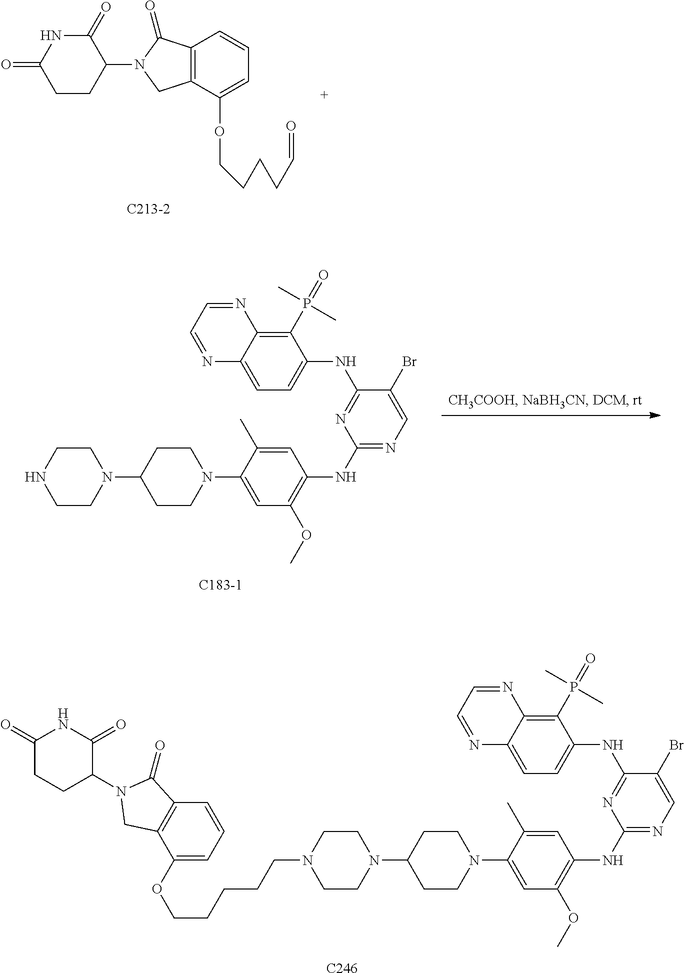 [SMILES: O=CCCCCOc1cccc2c1CN(C1CCC(=O)NC1=O)C2=O ; "C213-2" ; COc1cc(N2CCC(N3CCNCC3)CC2)c(C)cc1Nc1ncc(Br)c(Nc2ccc3nccnc3c2P(C)(C)=O)n1 ; "C183-1" ; COc1cc(N2CCC(N3CCN(CCCCCOc4cccc5c4CN(C4CCC(=O)NC4=O)C5=O)CC3)CC2)c(C)cc1Nc1ncc(Br)c(Nc2ccc3nccnc3c2P(C)(C)=O)n1 ; "C246"]

C213-2 (22 mg, 0.064 mmol) and C183-1 (or C158-1) (30.4 mg, 0.045 mmol) were dissolved in 3.0 mL of anhydrous DCM. CH₃COOH (5.8 mg, 0.096 mmol, dissolved in 0.5 mL of anhydrous DCM) was added dropwise under nitrogen protection. The mixture was stirred at room temperature for 30 min. Then NaBH₃CN (8.03 mg, 0.128 mmol) was added. The mixture was reacted with stirring at room temperature for 2 h. The mixture was purified by Prep-HPLC to afford yellow solid pure product C246 (8.0 mg, yield: 12.5%). LCMS: [M+H]⁺=1008, 1010.

### Preparation of 7-(5-(4-(1-(4-((5-chloro-4-((2-(dimethylphosphoryl)phenyl)amino)pyrimidin-2-yl)amino)-3-methoxyphenyl)piperidin-4-yl)piperazin-1-yl)pent-1-yn-1-yl)-2-(2,6-dioxopiperidin-3-yl)-3-oxoisoindoline-5-carbonitrile (C258)

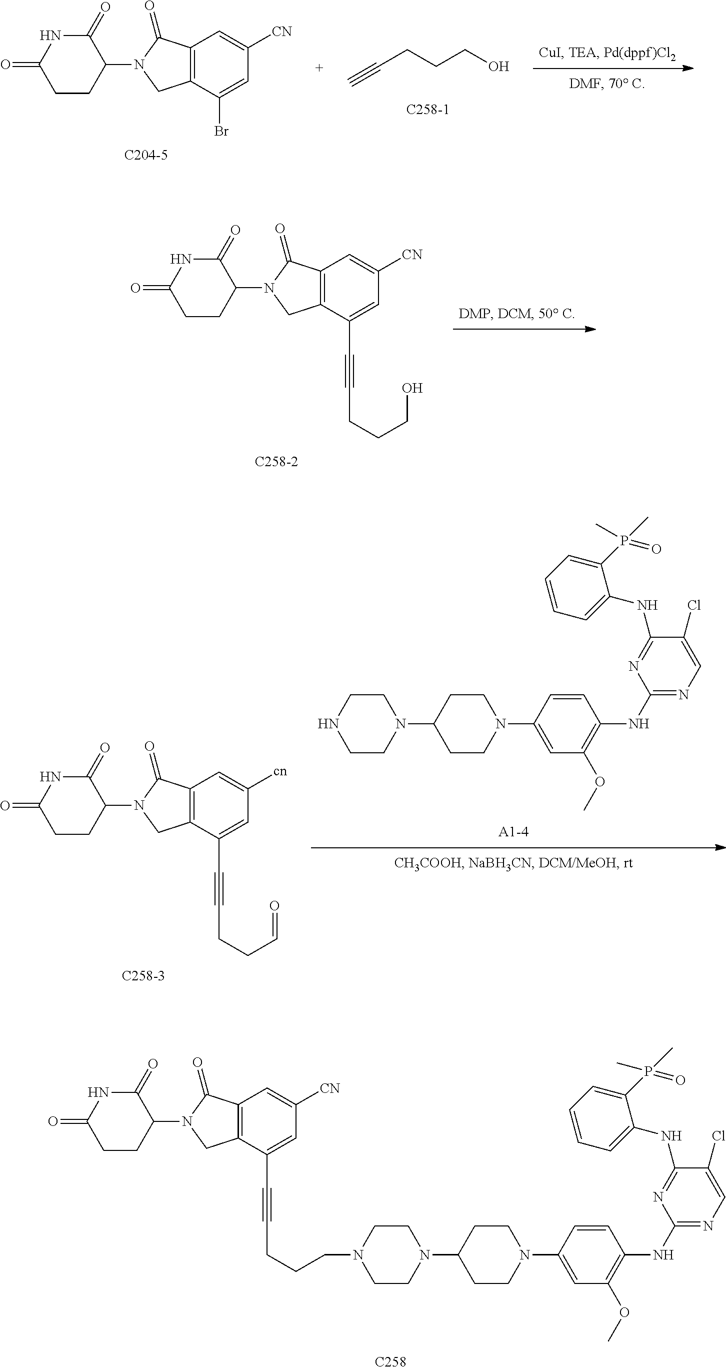

### Step 1

C204-5 (173.5 mg, 0.5 mmol), CuI (19.0 mg, 0.1 mmol) and Pd(dppf)Cl₂ (73.1 mg, 0.1 mmol) were dispersed in 10.0 mL of anhydrous DMF. C258-1 (105 mg, 2.5 mmol) and TEA (151.5 mg, 1.5 mmol) were added successively under N₂ protection. The mixture was heated to 70° C. and reacted for 20 h. LCMS showed that the reaction was completed. The mixture was cooled to room temperature and directly purified by RP-Flash chromatography to afford light-yellow solid product C258-2 (180 mg, 100%). LCMS: [M+H]⁺=352.

### Step 2

C258-2 (180 mg, 0.513 mmol) was dissolved in 90.0 mL of anhydrous DCM. Dess-Martin reagent (435 mg, 1.026 mmol) was added under N₂ protection. The mixture was heated to 50° C., and reacted under reflux for 2.0 h. TLC showed that the reaction was completed. The mixture was cooled to room temperature, and then 20 mL of saturated NaHCO₃ solution and 20 mL of saturated Na₂S₂O₃ solution were added. The mixture was stirred at room temperature for 5 min. The organic layer was separated, dried with anhydrous sodium sulfate, and filtered with suction. The filtrate was concentrated under reduced pressure to afford a crude product. The crude product was purified by Prep-TLC to afford light-yellow solid product C258-3 (105 mg, 58.3%). LCMS: [M+H]⁺=350.

### Step 3

C258-3 (93 mg, 0.266 mmol) and A1-4 (129 mg, 0.226 mmol) were dissolved in a mixture of 7.0 mL of anhydrous DCM and 0.7 mL of anhydrous MeOH. CH₃COOH (24 mg, 0.399 mmol) was added under nitrogen protection. The mixture was stirred at room temperature for 0.5 h. Then solid NaBH₃CN (33.5 mg, 0.532 mmol) was added. The mixture was further reacted at room temperature for 2 h. LCMS and TLC showed that the reaction was completed. The mixture was purified by Prep-HPLC to afford white solid pure product C258 (30 mg, yield: 12.5%). LCMS: [M+H]⁺=903.

### Preparation of 7-(6-(4-(1-(4-((5-chloro-4-((2-(dimethylphosphoryl)phenyl)amino)pyrimidin-2-yl)amino)-3-methoxyphenyl)piperidin-4-yl)piperazin-1-yl)hex-1-yn-1-yl)-2-(2,6-dioxopiperidin-3-yl)-3-oxoisoindoline-5-carbonitrile (C259)

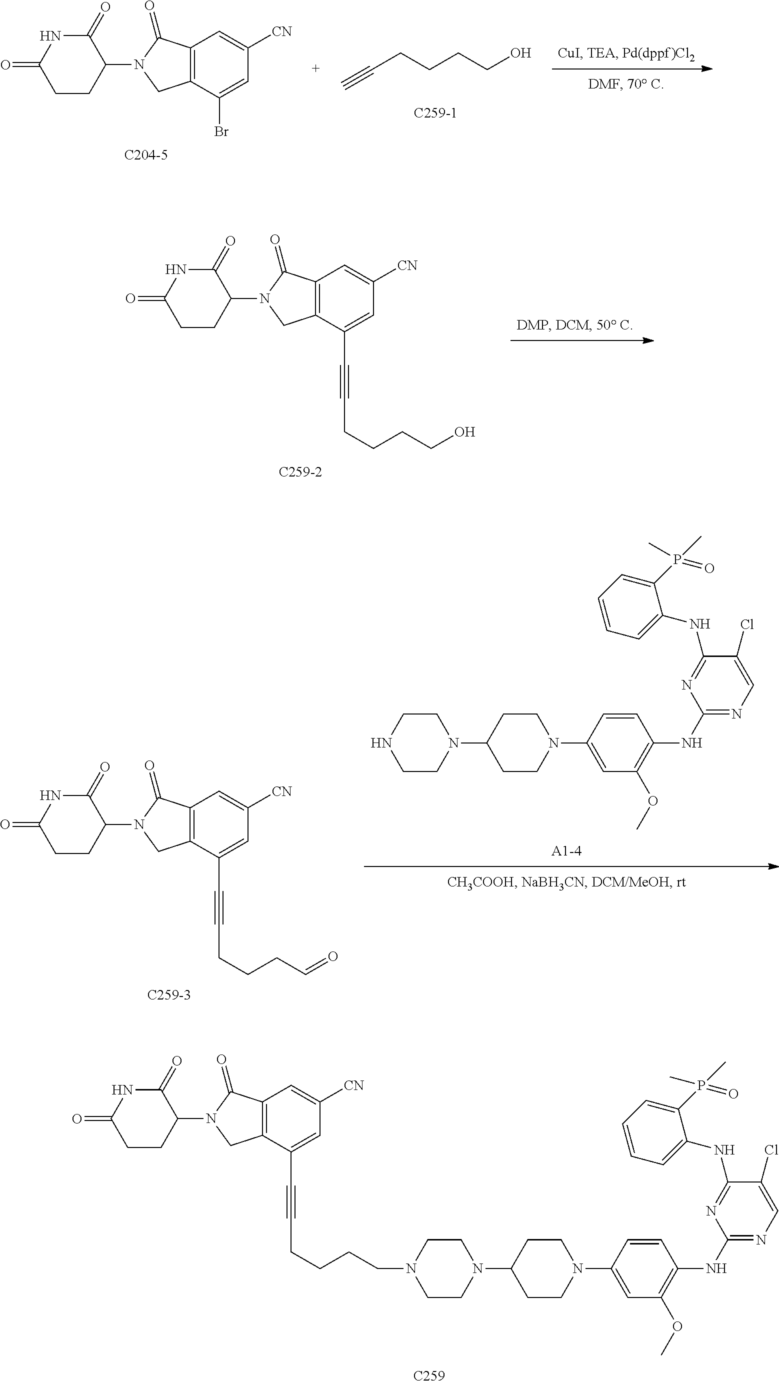

### Step 1

C204-5 (173.5 mg, 0.5 mmol), CuI (19.0 mg, 0.1 mmol) and Pd(dppf)Cl₂ (73.1 mg, 0.1 mmol) were dispersed in 10.0 mL of anhydrous DMF. C259-1 (122.5 mg, 2.5 mmol) and TEA (151.5 mg, 1.5 mmol) were added successively under N2 protection. The mixture was heated to 70° C. and reacted for 20 h. LCMS showed that the reaction was completed. The mixture was cooled to room temperature and directly purified by RP-Flash chromatography to afford light-yellow solid crude C259-2 (210 mg, 100%). LCMS: [M+H]⁺=366.

### Step 2

C259-2 (210 mg, 0.575 mmol) was dissolved in 105 mL of anhydrous DCM. Dess-Martin reagent (439 mg, 1.035 mmol) was added under N₂ protection. The mixture was heated to 50° C., and reacted under reflux for 2.0 h. TLC showed that the reaction was completed. The mixture was cooled to room temperature, and then 20 mL of saturated NaHCO₃ solution and 20 mL of saturated Na₂S₂O₃ solution were added. The mixture was stirred at room temperature for 5 min. The organic layer was separated, dried with anhydrous sodium sulfate, and filtered with suction. The filtrate was concentrated under reduced pressure to afford a crude product. The crude product was purified by Prep-TLC to afford light-yellow solid product C259-3 (130 mg, 62.5%). LCMS: [M+H]⁺=364.

### Step 3

C259-3 (120 mg, 0.33 mmol) and A1-4 (160 mg, 0.28 mmol) were dissolved in a mixture of 8.0 mL of anhydrous DCM and 0.8 mL of anhydrous MeOH. CH₃COOH (29.8 mg, 0.495 mmol) was added under nitrogen protection. The mixture was stirred at room temperature for 0.5 h. Then solid NaBH₃CN (41.5 mg, 0.66 mmol) was added. The mixture was further reacted at room temperature for 2 h. LCMS and TLC showed that the reaction was completed. The mixture was purified by Prep-HPLC to afford white solid pure product C259 (30 mg, yield: 9.93%). LCMS: [M+H]⁺=917.

### Preparation of 3-(4-(7-(4-(1-(4-((5-chloro-4-((2-(dimethylphosphoryl)phenyl)amino)pyrimidin-2-yl)amino)-3-methoxyphenyl)piperidin-4-yl)piperazin-1-yl)hept-1-yn-1-yl)-1-oxo-6-(trifluoromethyl)isoindolin-2-yl)piperidine-2,6-dione (C267)

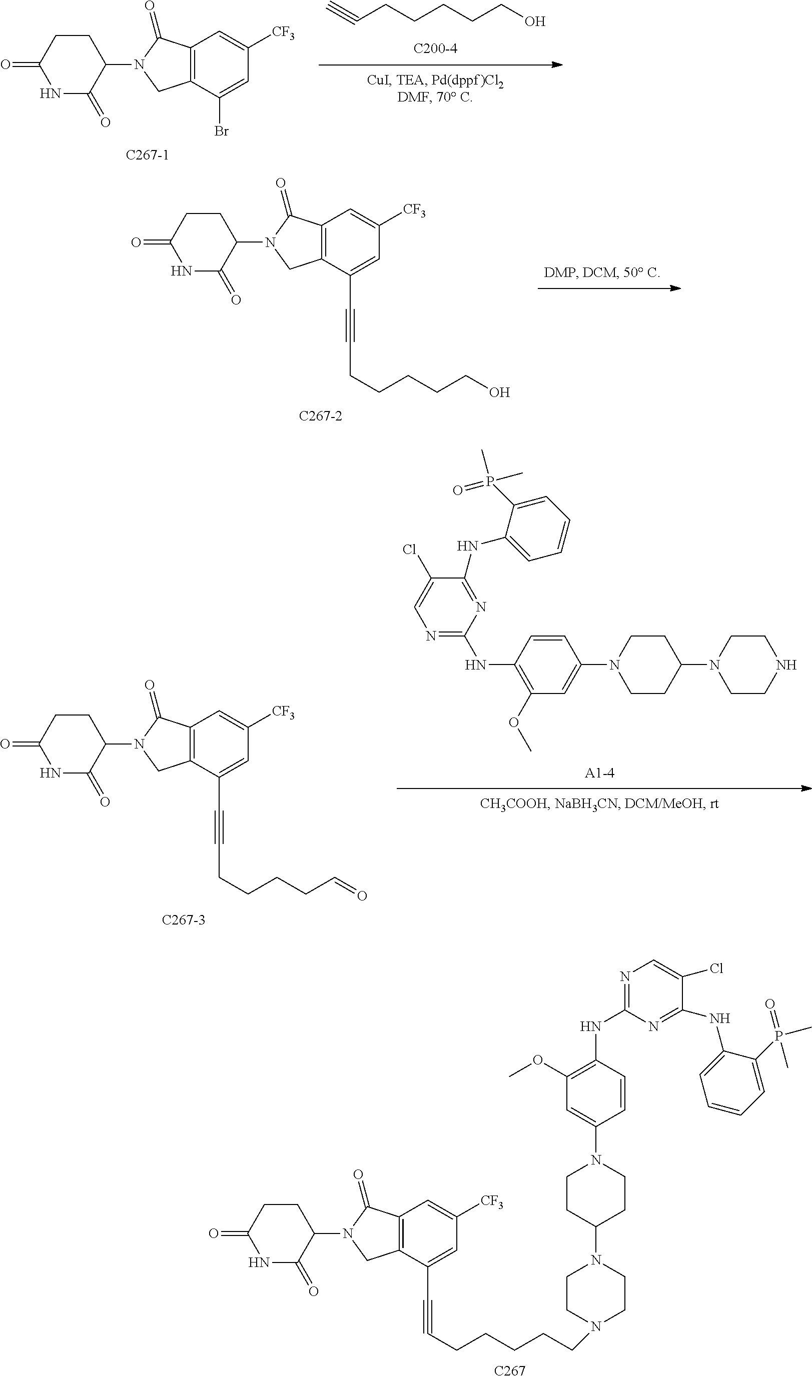

### Step 1

Compound C267-1 (or C200-5) (60 mg, 0.154 mmol), compound C200-4 (43 mg, 0.384 mmol), cuprous iodide (2.9 mg, 0.015 mmol), Pd(dppf)Cl₂ (11.3 mg, 0.015 mmol) and triethylamine (46.6 mg, 0.461 mmol) were dissolved in 2 mL of anhydrous DMF. The mixture was stirred at 70° C. under nitrogen protection overnight. The solvent was removed under reduced pressure. The crude product was purified by thin layer chromatography to afford compound C267-2 (61 mg, yield: 94.0%). LCMS: [M+H]⁺=423.

### Step 2

Compound C267-2 (81 mg, 0.192 mmol) and Dess-Martin reagent (163 mg, 0.384 mmol) were dissolved in 10 mL of dichloromethane and stirred at 50° C. for 2 h. The mixture was cooled to room temperature, and saturated sodium bicarbonate aqueous solution and saturated sodium thiosulfate aqueous solution were added successively and stirred for 5 minutes respectively to quench the reaction. The organic phase was separated, and the aqueous phase was extracted with dichloromethane for three times. The organic phases were combined, dried with anhydrous sodium sulfate, and filtered. The filtrate was concentrated, and the crude product was purified by TLC to afford a white solid compound C267-3 (60 mg, yield: 74.4%). LCMS: [M+H]⁺=421.

### Step 3

Compound 267-3 (20 mg, 0.048 mmol) and compound A1-4 (22 mg, 0.038 mmol) were dissolved in 2 mL of dichloromethane/methanol (2/1, v/v), and glacial acetic acid (2.9 mg, 0.038 mmol) was added dropwise. The mixture was stirred at room temperature under nitrogen protection for 1 h. Then sodium cyanobohydride (2.4 mg, 0.038 mmol) was added. The mixture was further stirred for 1 h. The solvent was removed under reduced pressure below 40° C., and the crude product was purified by Flash chromatography to afford off-white solid compound C267 (12 mg, yield: 46.3%). LCMS: [M+H]⁺=974. ¹H NMR (400 MHz, DMSO-d₆) δ 11.17 (s, 1H), 11.04 (s, 1H), 8.48 (s, 1H), 8.06 (d, 2H), 7.99-7.98 (m, 2H), 7.57-7.49 (m, 1H), 7.38 (d, 1H), 7.33 (d, 1H), 7.09 (t, 1H), 6.62 (d, 1H), 6.46 (dd, 1H), 5.32 (dd, 1H), 5.18 (dd, 1H), 4.58 (d, 1H), 4.43 (d, 1H), 3.76 (s, 3H), 3.70 (d, 2H), 2.93 (m, 1H), 2.69-2.60 (m, 4H), 2.52 (d, 2H), 2.46 (m, 3H), 2.44-2.26 (m, 8H), 1.99 (m, 4H), 1.82 (s, 1H), 1.78 (m, 3H), 1.75 (s, 3H), 1.60 (d, 2H), 1.46 (m, 3H).

### Preparation of 3-(4-(6-(4-(1-(4-((5-chloro-4-((2-(dimethylphosphoryl)phenyl)amino)pyrimidin-2-yl)amino)-3-methoxyphenyl)piperidin-4-yl)piperazin-1-yl)hex-1-yn-1-yl)-1-oxo-6-(trifluoromethyl)isoindolin-2-yl)piperidine-2,6-dione (C268)

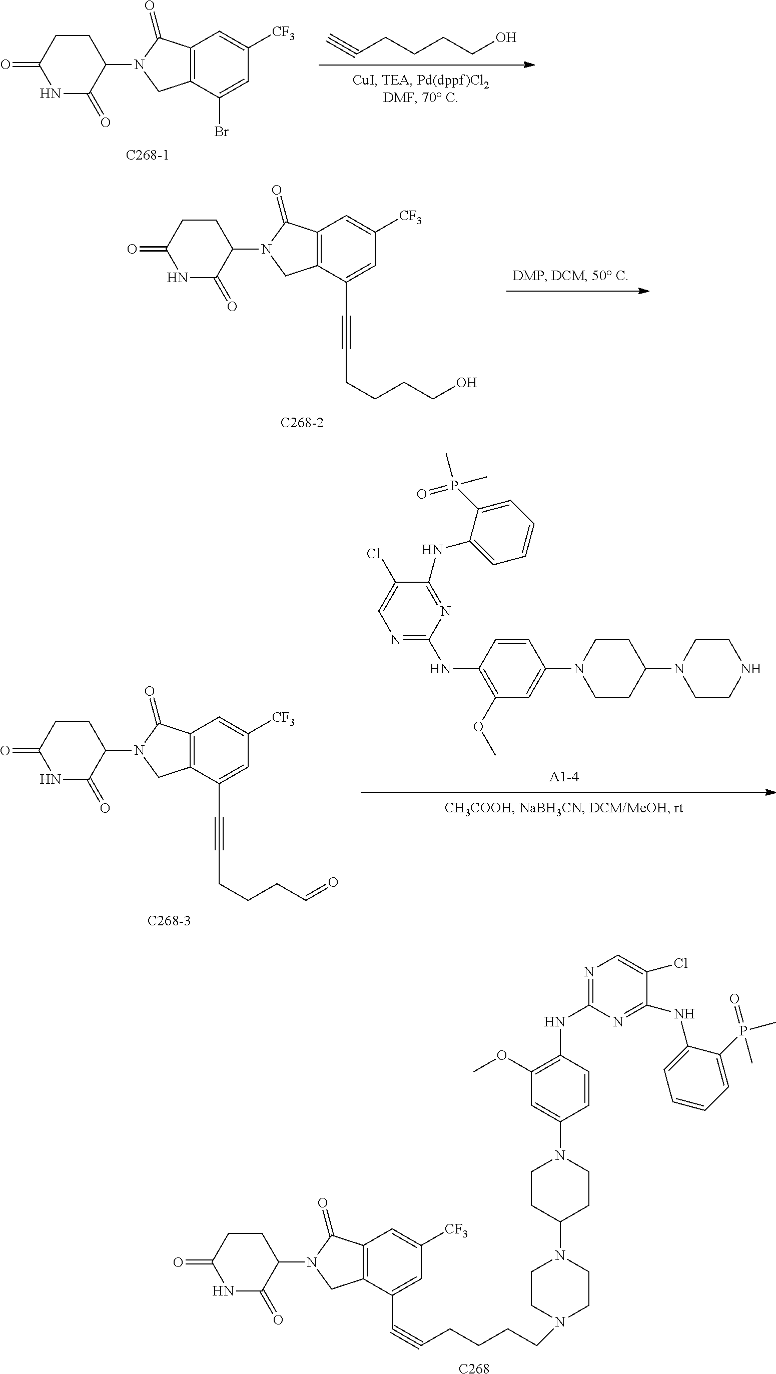

### Step 1

Compound C268-1 (or C200-5) (100 mg, 0.256 mmol), 5-alkynyl-1-hexanol (62.9 mg, 0.641 mmol), cuprous iodide (4.9 mg, 0.026 mmol), Pd(dppf)Cl₂ (18.8 mg, 0.026 mmol) and triethylamine (77.7 mg, 0.769 mmol) were dissolved in 3 mL of anhydrous DMF. The mixture was stirred at 70° C. under nitrogen protection overnight. The solvent was removed under reduced pressure, and the crude product was purified by thin layer chromatography to afford compound C268-2 (80 mg, yield: 76.5%). LCMS: [M+H]⁺=409.

### Step 2

Compound C268-2 (126 mg, 0.309 mmol) and Dess-Martin reagent (262 mg, 0.618 mmol) were dissolved in 10 mL of dichloromethane and stirred at 50° C. for 2 h. The mixture was cooled to room temperature, and saturated sodium bicarbonate aqueous solution and saturated sodium thiosulfate aqueous solution were added successively and stirred for 5 minutes respectively to quench the reaction. The organic phase was separated, and the aqueous phase was extracted with dichloromethane for three times. The organic phases were combined, dried with anhydrous sodium sulfate, and filtered. The filtrate was concentrated, and the crude product was purified by TLC to afford a white solid compound C268-3 (40 mg, yield: 32.0%). LCMS: [M+H]⁺=407.

### Step 3

Compound C268-3 (39 mg, 0.096 mmol) and compound A1-4 (38.3 mg, 0.067 mmol) were dissolved in 2 mL of dichloromethane/methanol (2/1, v/v), and glacial acetic acid (5.8 mg, 0.096 mmol) was added dropwise. The mixture was stirred at room temperature for 1 h under nitrogen protection. Then sodium cyanobohydride (4.2 mg, 0.067 mmol) was added. The mixture was further stirred for 1 h. The solvent was removed under reduced pressure below 40° C., and the crude product was purified by Flash chromatography to afford off-white solid compound C268 (30 mg, yield: 92.1%). LCMS: [M+H]⁺=960. ¹H NMR (400 MHz, DMSO-d₆) δ 11.17 (s, 1H), 11.04 (s, 1H), 8.48 (s, 1H), 8.06 (d, 2H), 8.01-7.98 (m, 2H), 7.53 (m, 1H), 7.38 (d, 1H), 7.33 (d, 1H), 7.09 (t, 1H), 6.62 (d, 1H), 6.47 (dd, 1H), 5.32 (m, 1H), 5.18 (dd, 1H), 4.57 (d, 1H), 4.42 (d, 1H), 3.76 (s, 3H), 3.71 (d, 2H), 2.93 (m, 1H), 2.70-2.62 (m, 3H), 2.53 (d, 3H), 2.38 (m, 3H), 2.30 (s, 4H), 2.05-1.96 (m, 4H), 1.85 (d, 2H), 1.78 (s, 3H), 1.75 (s, 3H), 1.63-1.58 (m, 4H), 1.53-1.46 (m, 2H).

### Preparation of 3-(4-(5-(4-(1-(4-((5-chloro-4-((2-(dimethylphosphoryl)phenyl)amino)pyrimidin-2-yl)amino)-3-methoxyphenyl)piperidin-4-yl)piperazin-1-yl)pent-1-yn-1-yl)-1-oxo-6-(trifluoromethyl)isoindolin-2-yl)piperidine-2,6-dione (C269)

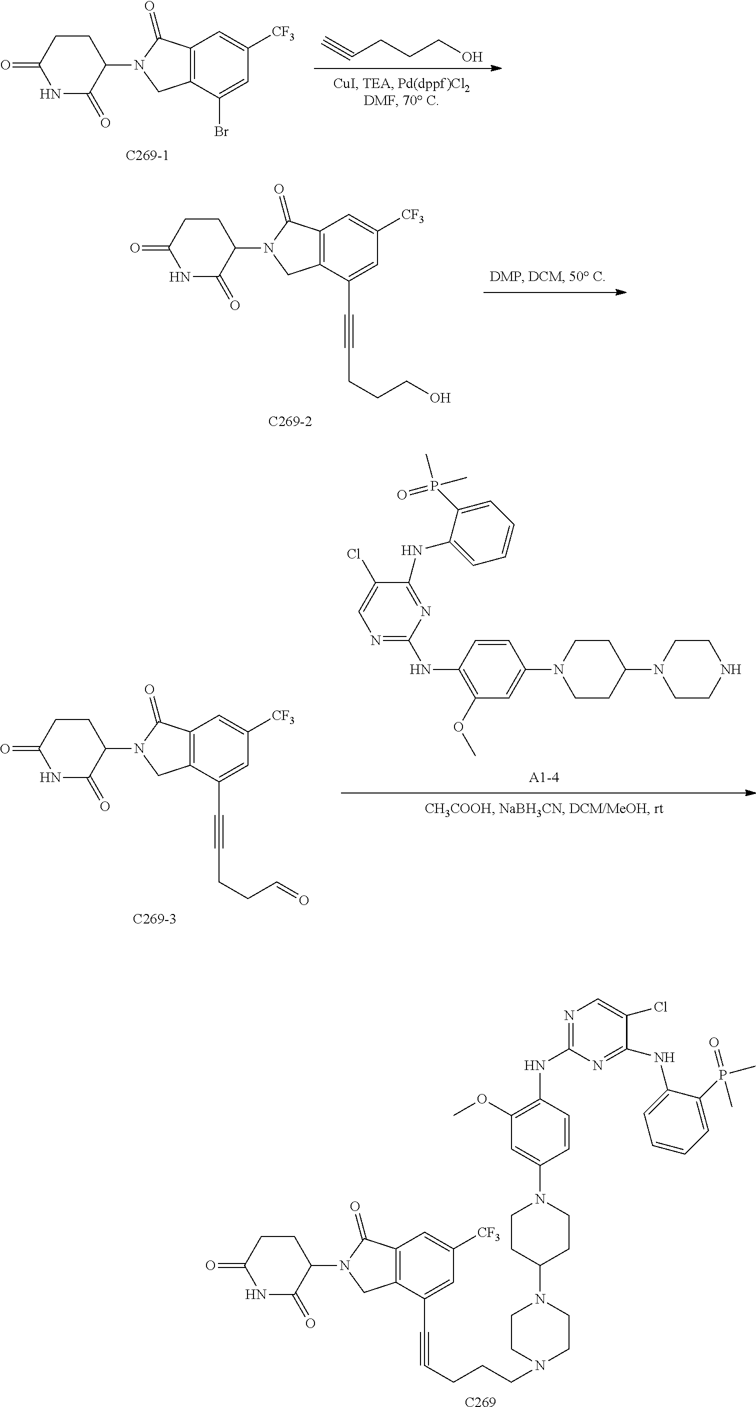

### Step 1

Compound C269-1 (or C200-5) (60 mg, 0.154 mmol), 4-alkynyl-1-pentanol (32.3 mg, 0.384 mmol), cuprous iodide (2.9 mg, 0.015 mmol), Pd(dppf)Cl₂ (11.3 mg, 0.015 mmol) and triethylamine (46.6 mg, 0.461 mmol) were dissolved in 2 mL of anhydrous DMF. The mixture was stirred at 70° C. under nitrogen protection overnight. The solvent was removed under reduced pressure, and the crude product was purified by thin layer chromatography to afford compound C269-2 (58 mg, yield: 95.8%). LCMS: [M+H]⁺=395.

### Step 2

Compound C269-2 (81 mg, 0.206 mmol) and Dess-Martin reagent (174 mg, 0.410 mmol) were dissolved in 10 mL of dichloromethane and stirred at 50° C. for 2 h. The mixture was cooled to room temperature, and saturated sodium bicarbonate aqueous solution and saturated sodium thiosulfate aqueous solution were added successively and stirred for 5 minutes respectively to quench the reaction. The organic phase was separated, and the aqueous phase was extracted with dichloromethane for three times. The organic phases were combined, dried with anhydrous sodium sulfate, and filtered. The filtrate was concentrated, and the crude product was purified by TLC to afford a white solid compound C269-3 (60 mg, yield: 74.4%). LCMS: [M+H]⁺=393.

### Step 3

C269-3 (31 mg, 0.079 mmol) and A1-4 (36 mg, 0.063 mmol) were dissolved in 2 mL of dichloromethane/methanol (2/1, v/v), and glacial acetic acid (4.7 mg, 0.079 mmol) was added dropwise. The mixture was stirred at room temperature under nitrogen protection for 1 h. Then sodium cyanobohydride (4.0 mg, 0.063 mmol) was added. The mixture was further stirred for 1 h. The solvent was removed under reduced pressure below 40° C., and the crude product was purified by Flash chromatography to afford off-white solid compound C269 (13 mg, yield: 17.4%). LCMS: [M+H]⁺=946. ¹H NMR (400 MHz, DMSO-d₆) δ 11.17 (s, 1H), 11.04 (s, 1H), 8.48 (s, 1H), 8.06 (d, 2H), 8.01-7.98 (m, 2H), 7.53 (dd, 1H), 7.38 (d, 1H), 7.33 (d, 1H), 7.09 (t, 1H), 6.63 (d, 1H), 6.47 (dd, 1H), 5.32 (t, 1H), 5.19 (dd, 1H), 4.58 (d, 1H), 4.42 (d, 1H), 3.76 (s, 3H), 3.72 (d, 2H), 2.93 (m, 1H), 2.70-2.62 (m, 4H), 2.54 (m, 2H), 2.52 (d, 3H), 2.41 (m, 4H), 1.99 (dt, 5H), 1.86 (d, 2H), 1.78 (s, 3H), 1.74 (s, 3H), 1.55-1.45 (m, 3H).

### Preparation of 3-(4-(5-(4-(1-(4-((5-bromo-4-((5-(dimethylphosphoryl)quinoxalin-6-yl)amino)pyrimidin-2-yl)amino)-5-methoxy-2-methylphenyl)piperidin-4-yl)piperazin-1-yl)pentyl)-1-oxoisoindolin-2-yl)piperidine-2,6-dione (C285)

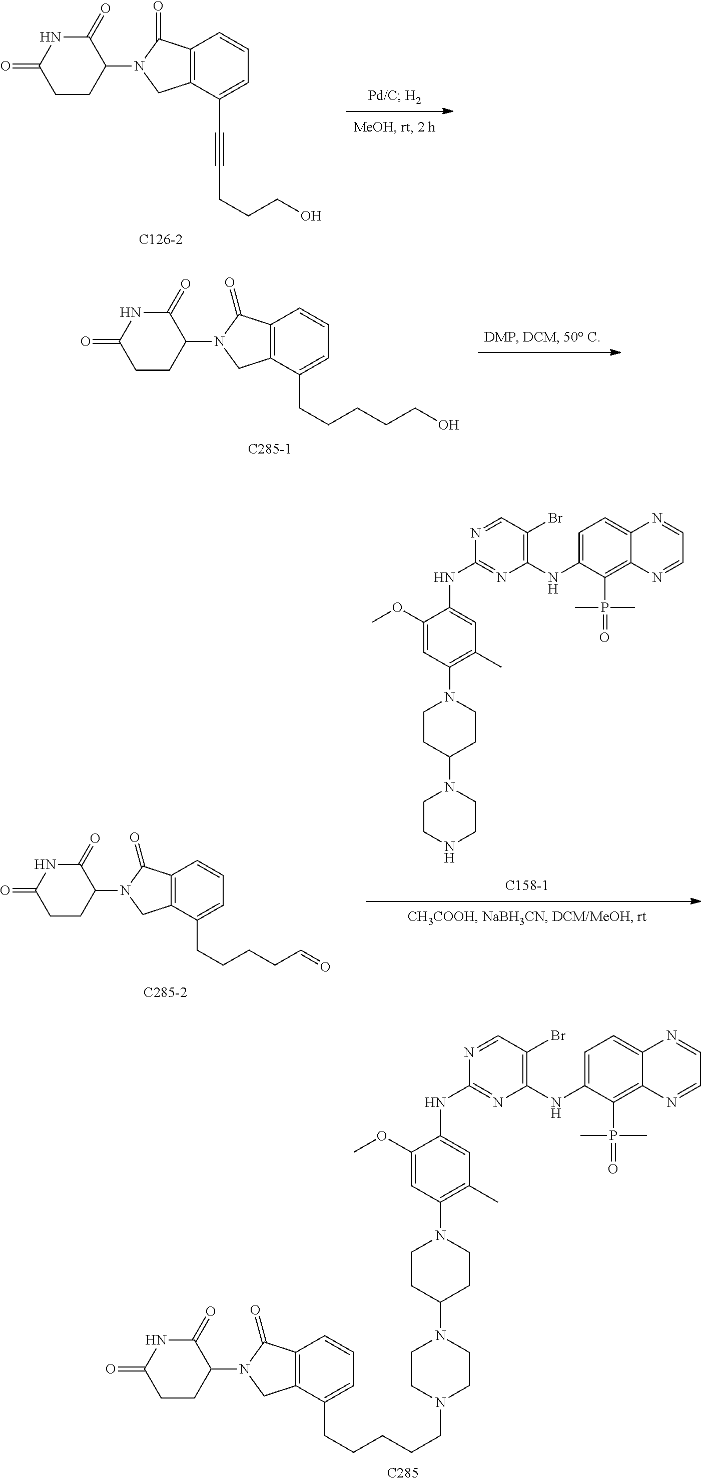

### Step 1

C126-2 (250 mg, 0.767 mmol) was dissolved in 100.0 mL of anhydrous MeOH, and 10% Pd/C (125 mg) was added to the mixture. H₂ (0.4 atm) was bubbled into the mixture. The mixture was reacted at room temperature for 2 h. LCMS showed that the reaction was completed. The mixture was filtered with suction to remove the solid catalyst. The solvent was removed from the filtrate under reduced pressure to afford white solid crude C285-1 (230 mg, 92%). LCMS: [M+H]⁺=331.

### Step 2

C285-1 (230 mg, 0.697 mmol) was dissolved in 150 mL of anhydrous DCM. Dess-Martin reagent (591 mg, 1.394 mmol) was added under N₂ protection. The mixture was heated to 50° C., and reacted under reflux for 2.0 h. TLC showed that the reaction was completed. The mixture was cooled to room temperature, and then 30 mL of saturated NaHCO₃ solution and 30 mL of saturated Na₂S₂O₃ solution were added. The mixture was stirred at room temperature for 5 min. The organic layer was separated, dried with anhydrous sodium sulfate, and filtered with suction. The filtrate was concentrated under reduced pressure to afford a crude product. The crude product was purified by Flash chromatography to afford white solid product C285-2 (210 mg, 91.3%). LCMS: [M+H]⁺=329.

### Step 3

C285-2 (49.2 mg, 0.15 mmol) and C158-1 (81.5 mg, 0.12 mmol) were dissolved in a mixture of 5.0 mL of anhydrous DCM and 0.5 mL of anhydrous MeOH. CH₃COOH (13.5 mg, 0.225 mmol) was added under nitrogen protection. The mixture was stirred at room temperature for 0.5 h. Then solid NaBH₃CN (18.8 mg, 0.3 mmol) was added. The mixture was further reacted at room temperature for 2 h. LCMS and TLC showed that the reaction was completed. The mixture was purified by Prep-HPLC to afford yellow solid pure product C285 (38 mg, yield: 25.6%). LCMS: [M+H]⁺=992, 994.

### Preparation of 3-(4-(6-(4-(1-(4-((5-bromo-4-((5-(dimethylphosphoryl)quinoxalin-6-yl)amino)pyrimidin-2-yl)amino)-5-methoxy-2-methylphenyl)piperidin-4-yl)piperazin-1-yl)hexyl)-1-oxoisoindolin-2-yl)piperidine-2,6-dione (C286)

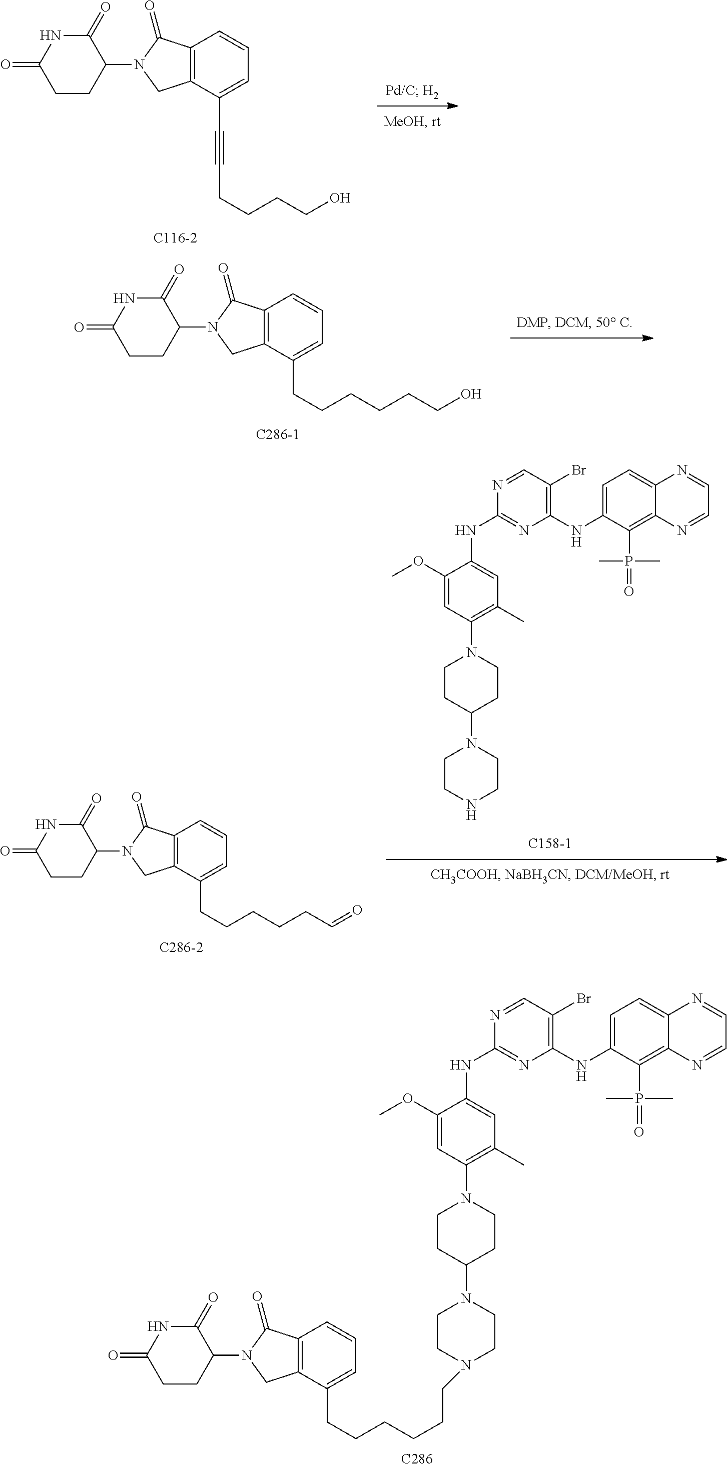

### Step 1

C116-2 (150 mg, 0.441 mmol) was dissolved in 60.0 mL of anhydrous Me H, and 100. Pd/C (150 mg) was added to the mixture. H₂ (0.4 atm) was bubbled into the mixture. The mixture was reacted at room temperature for 2 h. LCMS showed that the reaction was completed. The mixture was filtered with suction to remove the solid catalyst. The solvent was removed from the filtrate under reduced pressure to afford white solid crude C286-1 (150 mg, 98.50), which was directly used in the next step. LCMS: [M+H]==345.

### Step 2

C286-1 (150 mg, 0.436 mmol) was dissolved in 100 mL of anhydrous DCM. Dess-Martin reagent (370 mg, 0.872 mmol) was added under N₂ protection. The mixture was heated to 50° C., and reacted under reflux for 2.0 h. TLC showed that the reaction was completed. The mixture was cooled to room temperature, and then 20 mL of saturated NaHCO₃ solution and 20 mL of saturated Na₂S₂O₃ solution were added. The mixture was stirred at room temperature for 5 m2. The organic layer was separated, dried with anhydrous sodium sulfate, and filtered with suction. The filtrate was concentrated under reduced pressure to afford a crude product. The crude product was purified by Flash chromatography to afford white solid product C286-2 (100 mg, 66.70%). LCMS: [M+H]=343.

### Step 3

C286-2 (41 mg, 0.12 mmol) and C158-1 (65.2 mg, 0.096 mmol) were dissolved in a mixture of 4.0 mL of anhydrous DCM and 0.4 mL of anhydrous MeOH. CH₃COOH (10.8 mg, 0.18 mmol) was added under nitrogen protection. The mixture was stirred at room temperature for 0.5 h. Then solid NaBH₃CN (15.1 mg, 0.24 mmol) was added. The mixture was further reacted at room temperature for 2 h. LCMS and TLC showed that the reaction was completed. The mixture was purified by Prep-HIPLC to afford yellow solid pure product C286 (20 mg, yield: 16.6%). LCMS: [M+H]⁺=1006, 1008.

### Preparation of 3-(4-(6-(9-(4-((5-bromo-4-((5-(dimethylphosphoryl)quinoxalin-6-yl)amino)pyrimidin-2-yl)amino)-5-methoxy-2-methylphenyl)-3,9-diazaspiro[5.5]undecan-3-yl)hexyl)-1-oxoisoindolin-2-yl)piperidine-2,6-dione (C299)

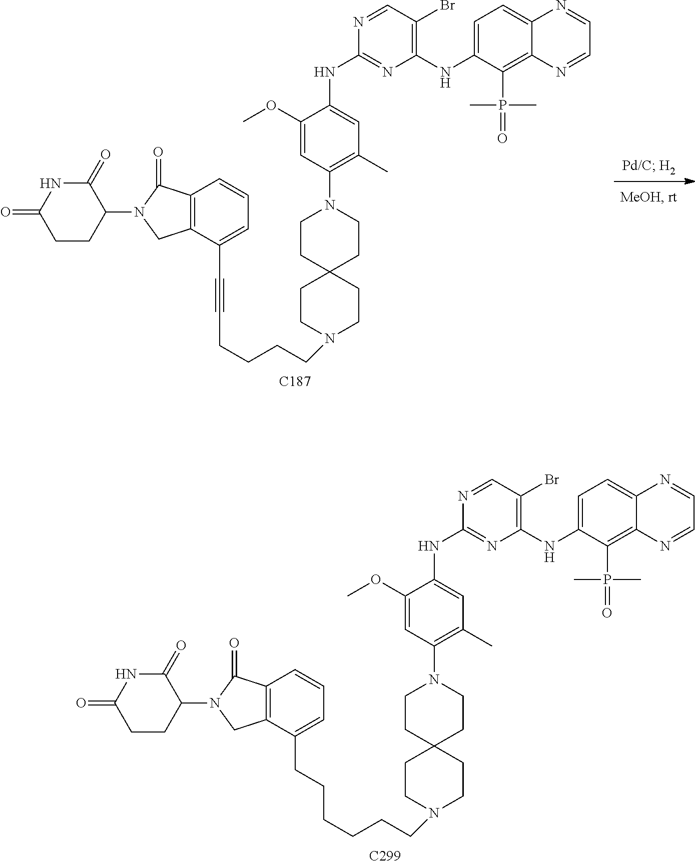

C187 (20 mg, 0.02 mmol) was dissolved in 20.0 mL of MeOH, and 10% Pd/C (10 mg) was added. 0.4 atm of H₂ was bubbled into the system. The mixture was reacted at room temperature for 2 h. LCMS showed that the reaction was completed. The mixture was filtered to remove the solid catalyst. The filtrate was concentrated to afford a crude product. The crude product was directly purified by RP-Flash chromatography to afford light-yellow solid crude product, which was further purified by Prep-HPLC to afford white solid product C299 (2.5 mg, yield: 12.6%). LCMS: [M+H]⁺=991, 993.

### Preparation of 3-(4-(4-(4-(1-(4-((5-bromo-4-((5-(dimethylphosphoryl)quinoxalin-6-yl)amino)pyrimidin-2-yl)amino)-5-methoxy-2-methylphenyl)piperidin-4-yl)piperazin-1-yl)butyl)-1-oxoisoindolin-2-yl)piperidine-2,6-dione (C301)

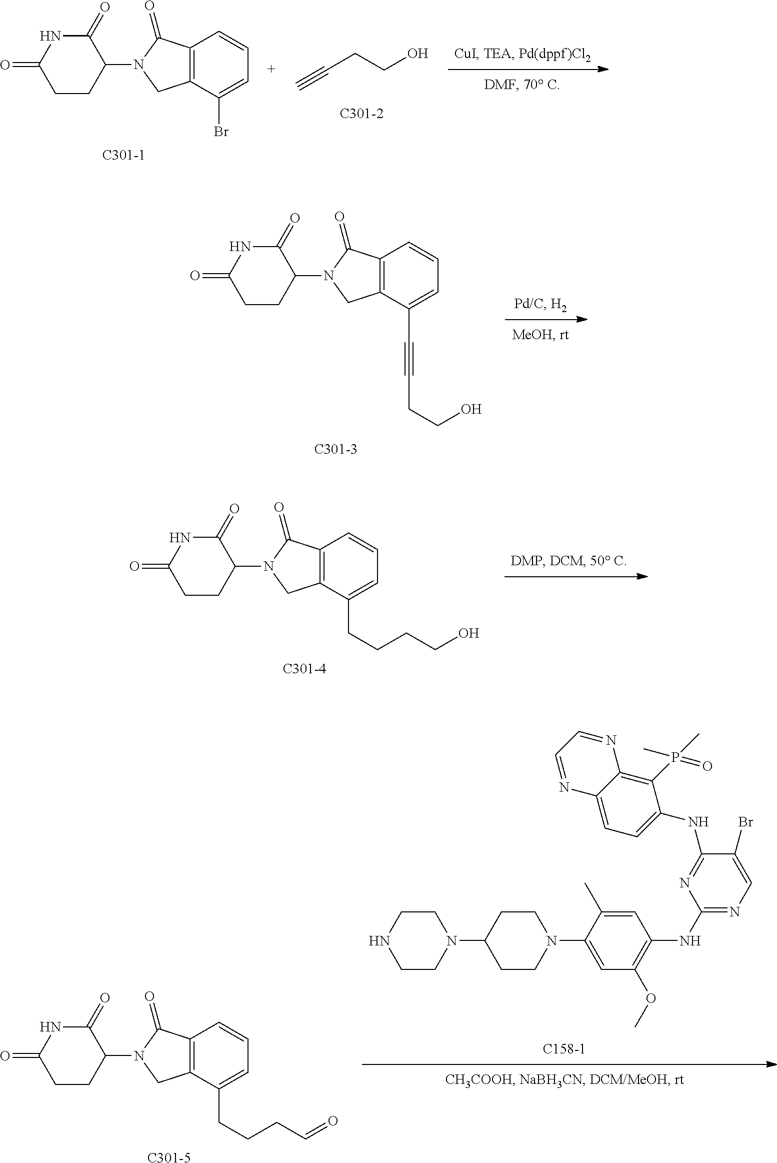

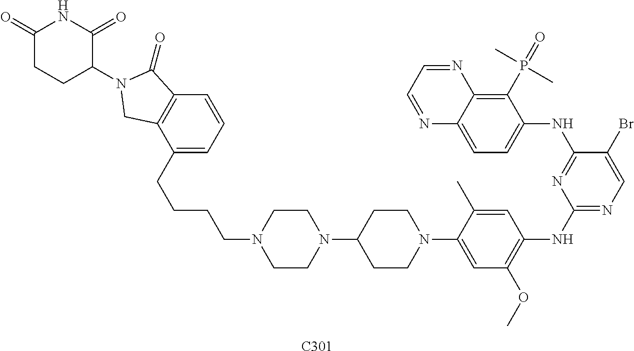 [SMILES: COc1cc(N2CCC(N3CCN(CCCCc4cccc5c4CN(C4CCC(=O)NC4=O)C5=O)CC3)CC2)c(C)cc1Nc1ncc(Br)c(Nc2ccc3nccnc3c2P(C)(C)=O)n1 ; "C301"]

### Step 1

C301-1 (644 mg, 2.0 mmol), CuI (38 mg, 0.2 mmol) and Pd(dppf)Cl₂ (146.2 mg, 0.2 mmol) were dispersed in 20.0 mL of anhydrous DMF. C301-2 (280.4 mg, 4.0 mmol) and TEA (606 mg, 6.0 mmol) were added successively under N2 protection. The mixture was heated to 70° C. and reacted for 20 h. LCMS showed that the reaction was completed. The mixture was cooled to room temperature and purified by RP-Flash chromatography to afford light-yellow solid crude C301-3 (450 mg, 72.1%). LCMS: [M+H]⁺=313.

### Step 2

C301-3 (450 mg, 1.44 mmol) was dissolved in 90.0 mL of anhydrous MeOH, and 10% Pd/C (225 mg) was added to the mixture. H₂ (0.4 atm) was bubbled into the mixture. The mixture was reacted at room temperature for 2 h. LCMS showed that the reaction was completed. The mixture was filtered with suction to remove the solid catalyst. The solvent was removed from the filtrate under reduced pressure to afford white solid crude C301-4 (450 mg, 98.9%), and the crude product was directly used in the next step. LCMS: [M+H]⁺=317.

### Step 3

C301-4 (420 mg, 1.33 mmol) was dissolved in 150 mL of anhydrous DCM. Dess-Martin reagent (1010 mg, 2.39 mmol) was added under N₂ protection. The mixture was heated to 50° C., and reacted under reflux for 2.0 h. TLC showed that the reaction was completed. The mixture was cooled to room temperature, and then 30 mL of saturated NaHCO₃ solution and 30 mL of saturated Na₂S₂O₃ solution were added. The mixture was stirred at room temperature for 5 min. The organic layer was separated, dried with anhydrous sodium sulfate, and filtered with suction. The filtrate was concentrated under reduced pressure to afford a crude product. The crude product was purified by Flash chromatography to afford white solid product C301-5 (350 mg, 83.3%). LCMS: [M+H]⁺=315.

### Step 4

C301-5 (47.1 mg, 0.15 mmol) and C158-1 (81.5 mg, 0.12 mmol) were dissolved in a mixture of 5.0 mL of anhydrous DCM and 0.5 mL of anhydrous MeOH. CH₃COOH (13.5 mg, 0.225 mmol) was added under nitrogen protection. The mixture was stirred at room temperature for 0.5 h. Then solid NaBH₃CN (18.8 mg, 0.3 mmol) was added. The mixture was further reacted at room temperature for 2 h. LCMS and TLC showed that the reaction was completed. The mixture was purified by RP-Flash to afford yellow solid pure product C301 (35 mg, yield: 23.8%). LCMS: [M+H]⁺=978, 980.

### Preparation of 3-(4-(4-(9-(4-((5-bromo-4-((5-(dimethylphosphoryl)quinoxalin-6-yl)amino)pyrimidin-2-yl)amino)-5-methoxy-2-methylphenyl)-3,9-diazaspiro[5.5]undecan-3-yl)butyl)-1-oxoisoindolin-2-yl)piperidine-2,6-dione (C302)

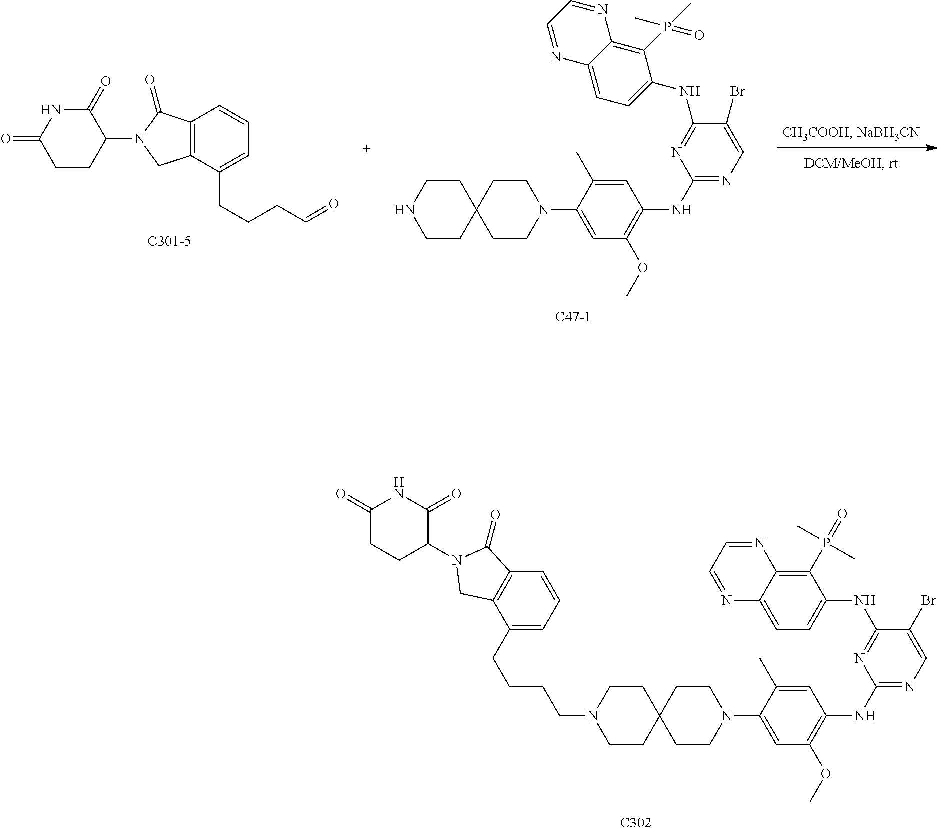 [SMILES: O=CCCCc1cccc2c1CN(C1CCC(=O)NC1=O)C2=O ; "C301-5" ; COc1cc(N2CCC3(CCNCC3)CC2)c(C)cc1Nc1ncc(Br)c(Nc2ccc3nccnc3c2P(C)(C)=O)n1 ; "C47-1" ; COc1cc(N2CCC3(CCN(CCCCc4cccc5c4CN(C4CCC(=O)NC4=O)C5=O)CC3)CC2)c(C)cc1Nc1ncc(Br)c(Nc2ccc3nccnc3c2P(C)(C)=O)n1 ; "C302"]

C301-5 (47.1 mg, 0.15 mmol) and C47-1 (79.7 mg, 0.12 mmol) were dissolved in a mixture of 5.0 mL of anhydrous DCM and 0.5 mL of anhydrous MeOH. CH₃COOH (13.5 mg, 0.225 mmol) was added under nitrogen protection. The mixture was stirred at room temperature for 0.5 h. Then solid NaBH₃CN (18.8 mg, 0.3 mmol) was added. The mixture was further reacted at room temperature for 2 h. LCMS and TLC showed that the reaction was completed. The mixture was directly purified by Prep-TLC to afford a crude product, which was further purified by RP-Flash to afford yellow solid pure product C302 (35 mg, yield: 24.3%). LCMS: [M+H]⁺=963, 965.

### Preparation of 3-(4-(3-(2-(9-(4-((5-bromo-4-((5-(dimethylphosphoryl)quinoxalin-6-yl)amino)pyrimidin-2-yl)amino)-5-methoxy-2-methylphenyl)-3,9-diazaspiro[5.5]undecan-3-yl)ethoxy)prop-1-yn-1-yl)-6-fluoro-1-oxoisoindolin-2-yl)piperidine-2,6-dione (C310)

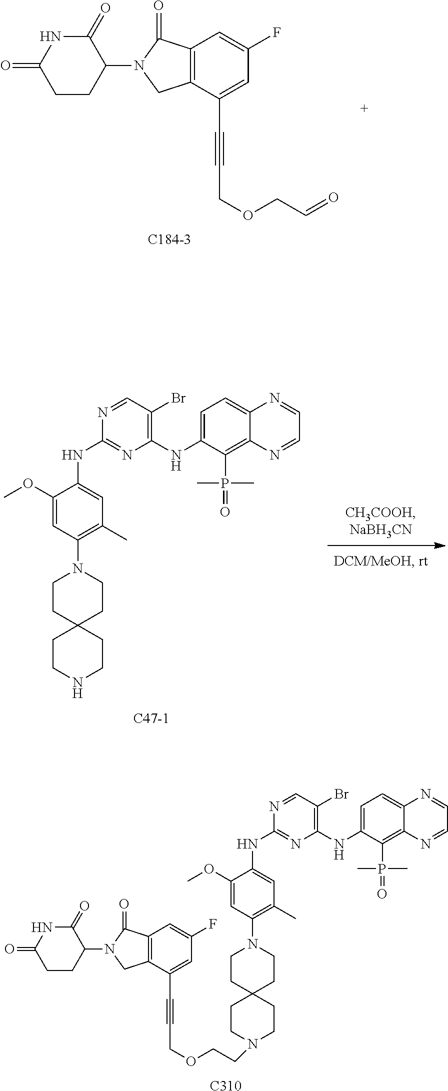

C184-3 (40 mg, 0.112 mmol) and C47-1 (55.6 mg, 0.084 mmol) were dissolved in a mixture of 5.0 mL of anhydrous DCM and 0.5 mL of anhydrous methanol. CH₃COOH (10.08 mg, 0.168 mmol) was added under nitrogen protection. The mixture was stirred at room temperature for 0.5 h. Then solid NaBH₃CN (14.1 mg, 0.224 mmol) was added. The mixture was further reacted at room temperature for 2 h. LCMS and TLC showed that the reaction was completed. The mixture was purified by Prep-HPLC to afford yellow solid pure product C310 (21 mg, yield: 18.8%). LCMS: [M+H]⁺=1007, 1009. ¹H-NMR: (400 MHz, DMSO-d₆) δ 11.026 (s, 1H), 8.865 (dd, J=2.0 Hz, 9.6 Hz, 3H), 8.265 (s, 2H), 7.938 (d, J=9.6 Hz, 1H), 7.676-7.611 (m, 3H), 7.324 (s, 1H), 6.810 (s, 1H), 5.187 (dd, J=5.2 Hz, 13.2 Hz, 1H), 4.470 (s, 3H), 4.360 (d, J=17.6 Hz, 1H), 3.782 (s, 3H), 3.677 (t, J=5.6 Hz, 2H), 2.991-2.913 (m, 1H), 2.8891-2.784 (m, 4H), 2.681-2.503 (m, 4H), 2.460-2.426 (m, 5H), 2.076 (s, 3H), 2.040 (s, 3H), 2.004 (s, 3H), 1.561-1.521 (m, 8H).

EGFR Inhibitory Activity Assay

1. Assay method

(1) A compound stock solution was prepared and diluted 3× to obtain a compound dilution; 10 nL of the compound dilution was transferred to a 384-well plate (784075, Greiner) by Echo 550;

(2) The plate was sealed, and centrifuged at 1,000 g for 1 min;

(3) 2×EGFR^{Dell9/T790M/C797S} and EGFR^{L858R/T790M/C797S} protein working solutions were prepared with 1× kinase buffer, respectively;

(4) 5 μl of the 2×EGFR protein working solution was added to the 384-well plate from step (2), centrifuged at 1,000 g for 30 s, and allowed to stand at room temperature (mixed thoroughly) for 10 min;

(5) A mixture of 2×TK-substrate-biotin (2 μM) and ATP was prepared with 1× Kinase buffer;

(6) 5 L of the TK-substrate-biotin and ATP (the mixture prepared in step (5)) was added to the 384-well plate from step (4) to initiate the reaction;

(7) The mixture was centrifuged at 1,000 g for 30 s; the plate was sealed, and allowed to stand (and reacted) at room temperature for 40 min;

(8) 4×Sa-XL 665 and TK-antibody-Cryptate were prepared with detection buffer;

(9) 5 L of the Sa-XL 665 and 5 L of the TK-antibody-Cryptate were added successively to the 384-well plate from step (7);

(10) The mixture was centrifuged at 1,000 g for 30 s, and allowed to stand (reacted) at room temperature for 1 h;

(11) The fluorescence values were read at 615 nm and 665 nm by enzyme labeling instrument (PerkinElmer, 74785).

2. Data analysis

The ratio (665/615) of each well was calculated.

Formula of inhibition rate (%):

$$Inhibition ( \% ) = [ 1 - \frac{{Ratio}_{cmpd} - {\overline{Ratio}}_{positive}}{{\overline{Ratio}}_{vehicle} - {\overline{Ratio}}_{positive}} ] \times 100$$

Ratio_{cmpd}: Ratio (665/615) value of assay compound.

R̅a̅t̅i̅o̅ₚₒₛᵢₜᵢᵥₑ: Average ratio (665/615) value of positive control.

R̅a̅t̅i̅o̅_{vehicle}: Average ratio (665/615) value of negative control.

The nonlinear regression curve (dose response-variable slope) between the value of inhibition rate (%) and the logarithm of compound concentration was fitted with graphpad prism 8.0, the effect dose curve of compound was drawn, and the IC₅₀ value was calculated.

3. Assay result

The IC₅₀ values of the inhibitory activity of the compound of the present disclosure on mutant EGFR^{Dell9/T790M/C797S} and EGFR^{58R/IT790M/C797S} are shown in the table below.

| | IC₅₀^{#} (nM) | |
|---|---|---|
| Compound No. | EGFR^{Del19/T790M/C797S} | EGFR^{L858R/T790M/C797S} |
| A1 | B | A |
| A2 | B | A |
| A3 | A | A |
| A6 | | A |
| A8 | A | A |
| A10 | A | A |
| A11 | A | A |
| A12 | | A |
| A13 | | B |
| A17 | A | A |
| A18 | A | A |
| C1 | | A |
| C4 | | B |
| C6 | | B |
| C25 | | B |
| C78 | | A |
| C88 | | A |
| C93 | | A |
| C94 | | A |
| C99 | | A |
| C101 | | A |
| C103 | | C |
| C104 | | A |
| C106 | | A |
| C107 | | A |
| C109 | | A |
| C110 | | A |
| C112 | | A |
| C116 | A | A |
| C148 | | A |
| C153 | A | A |
| C164 | | A |
| C167 | A | A |
| C176 | | A |
| C177 | A | A |
| C178 | A | A |
| C187 | A | A |
| C192 | A | A |
| Osimertinib | C | D |

^{#}Note:
A represents IC₅₀ of 1-100 nM, B represents IC₅₀ of 101-200 nM, C represents IC₅₀ of 201-300 nM, and D represents IC₅₀ of >300 nM.

Conclusion: the compounds of the present disclosure have strong inhibitory activity on mutant EGFR^{Dell9/T790M/C797S} and EGFR^{58R/IT790M/C797S}.

Assay of activity on Ba/F3(EGFR^{Dell9/T790M/C797S}) and Ba/F3(EGFR^{58R/IT790M/C797S}) cells

1. Assay Method

(1) Ba/F3(EGFR^{Dell9/T790M/C797S}) and Ba/F3(EGFR^{58R/IT790M/C797S}) cells were respectively cultured according to the requirements of ATCC, incubated in an incubator at 37° C. and 5% CO₂, and analyzed by index; cells with viability of >90% can be used in the assay, and cells were seeded in a 384-well plate (PerkinElmer, 6007680) with 700 cells/well, 30 μL/well.

(2) A compound stock solution was prepared, and then diluted 3× to obtain a compound dilution. 30 nL of the compound dilution was added to a 384-well plate by Echo (Labcyte, Echo550). The cells were incubated in an incubator at 37° C. and 5% CO₂ for 72 h.

(3) 30 μL of CTG was added to each well, and the 384-well plate was shaken on a Plate shaker (QILINBEIER, QB-9002). The 384-well plate was incubated at 37° C. and 5% CO₂ in the dark for 30 min, and the chemiluminescence value was read by Envision (PerkinElmer, EnVision 2104).

2. Data analysis

The percent inhibition rate (% inhibition) was calculated by the following formula

$$Inhibition ( \% ) = [ 1 - \frac{{LUM}_{cmpd} - {\overline{LUM}}_{positi ve}}{{\overline{LUM}}_{vehicle} - {\overline{LUM}}_{positive}} ] \times 100$$

L̅U̅M̅_{cmpd}: Luminescence value of assay compound.

L̅U̅M̅ₚₒₛᵢₜᵢᵥₑ: Average LUM value of positive drug at a concentration of 10 μM.

L̅U̅M̅_{vehicle}. Average LUM value of negative control group without drug treatment.

The nonlinear regression curve (dose response-variable slope) between the value of inhibition rate (%) and the logarithm of compound concentration was fitted with graphpad prism 8.0, the effect dose curve of compound was drawn, and the IC₅₀ value was calculated.

Y=Bottom+(Top−Bottom)/(1+1\hat{ }((LogIC₅₀−X)*HillSlope))

X-axis: logarithm of compound concentration; Y axis: inhibition rate (00 inhibition).

3. Assay result

The IC50 values of the inhibitory activity of the compound of the present disclosure on Ba/F3(EGFR^{Dell9/T790M/C797S}) and Ba/F3(EGFR^{58R/IT790M/C797S}) cells are shown in the table below.

| | IC₅₀^{#} (nM) | |
|---|---|---|
| Compound | Ba/F3 | Ba/F3 |
| No. | (EGFR^{Del19/T790M/C797S}) | (EGFR^{L858R/T790M/C797S}) |
| A1 | | C |
| A2 | | C |
| A10 | A | A |
| A11 | A | B |
| A12 | | A |
| A13 | | B |
| C1 | | A |
| C4 | | B |
| C6 | | C |
| C25 | | C |
| C47 | B | B |
| C78 | | D |
| C88 | | D |
| C93 | | D |
| C94 | | D |
| C99 | | D |
| C101 | | D |
| C103 | | |
| C104 | | D |
| C106 | | D |
| C107 | | B |
| C109 | | D |
| C110 | | D |
| C116 | A | |
| C126 | A | |
| C148 | | D |
| C150 | | D |
| C153 | A | A |
| C158 | A | B |
| C162 | D | D |
| C163 | A | |
| C164 | A | |
| C166 | A | B |
| C167 | | A |
| C169 | A | A |
| C171 | A | |
| C172 | A | |
| C173 | A | |
| C174 | A | A |
| C176 | | A |
| C177 | A | |
| C178 | A | |
| C179 | A | |
| C181 | A | |
| C182 | A | |
| C183 | A | A |
| C184 | A | |
| C185 | A | |
| C186 | A | |
| C187 | A | |
| C188 | A | |
| C189 | A | |
| C190 | A | |
| C191 | A | |
| C192 | A | |
| C193 | A | |
| C194 | A | |
| C195 | A | |
| C196 | A | |
| C200 | B | |
| C202 | A | |
| C204 | A | |
| C211 | A | |
| C212 | A | |
| C213 | A | A |
| C214 | A | A |
| C215 | A | |
| C218 | A | |
| C227 | A | |
| C228 | A | |
| C229 | A | |
| C235 | A | A |
| C236 | A | |
| C238 | A | |
| C240 | A | |
| C243 | A | |
| C244 | A | A |
| C245 | A | |
| C246 | A | A |
| C258 | A | |
| C259 | A | |
| C267 | A | |
| C268 | A | |
| C269 | A | |
| C285 | A | |
| C286 | A | |
| C299 | A | A |
| C301 | A | |
| C302 | A | |
| C310 | A | |
| A1-4 | D | D |
| C47-1 | | B |
| C158-1 | A | |
| Osimertinib | D | D |

^{#}Note:
A represents IC₅₀ of 1-100 nM, B represents IC₅₀ of 101-200 nM, C represents IC₅₀ of 201-300 nM, and D represents IC₅₀ of >300 nM.

Conclusion: The compounds of the present disclosure have strong inhibitory activity on mutant Ba/F3(EGFR^{Dell9/T790M/C797S}) and Ba/F3(EGFR^{58R/IT790M/C797S}) cells.

Assay of activity of compounds to induce EGFR^{58R/IT790M/C797S} and EGFR^{Dell9/T790M/C797S} protein degradation

In order to further explain the reason why the compounds of the present disclosure have inhibitory activity on Ba/F3(EGFR^{58R/IT790M/C797S}) and Ba/F3(EGFR^{Dell9/T790M/C797S}) cells, representative compounds C176 and C213 were selected to study the mechanism of action of the compounds, and observe their effects on EGFR^{58R/IT790M/C797S} and EGFR^{Dell9/T790M/C797S} protein levels.

(1) Cell culture:

Ba/F3(EGFR^{58R/IT790M/C797S}) and Ba/F3(EGFR^{Dell9/T790M/C797S}) cells were cultured according to the culture conditions recommended by ATCC, and analyzed by index.

Complete medium: 1640 medium, 10% FBS, 1× glutamine, 1× penicillin-streptomycin.

Culture conditions: incubated at 37° C., 95% air, 5% CO₂ incubator.

(2) Compound stock solution: 10 mM stock solution in DMSO, stored at −20° C.

(3) Preparation of cell suspension:

The cells in the cell culture bottle were collected, and the cells with viability of >90% can be used in the assay. Cells were seeded in a 96-well plate with 40 μL cells, 1*10⁵ cells/well.

(4) Compound treatment:

Compound was diluted 3× with DMSO, starting at 1.0 mM (for EGFR^{58R/IT790M/C797S} assay) and 5 mM (for EGFR^{Dell9/T790M/C797S} assay), respectively, into 10 concentrations to prepare working solutions.

(5) Compound was pipetted into the 96-well plate to treat cells in a 37° C., 95% air, 5% CO₂ incubator for 24 h.

(6) Detection:

1) 1 μg/mL of EGF activated cells were treated for 10 min;

2) After the compound treatment, lysis buffer was added to lyse cells; 10 μL of cell lysate was transferred to a 384-well plate; in addition, control lysate and negative control plus 5 μL of acceptor mix were added to the 384-well plate, and shaken on a shaker for 1-2 min;

3) 5 μL of donor mix was added to each well; the 384-well plate was sealed, shaken on a shaker for 1-2 min, left in the dark at room temperature overnight, and read with an enzyme labeling instrument.

(7) Data analysis:

Alpha counts were fitted by logarithmic treatment of compound concentration with Graphpad Prism 8.0.

Y=Bottom+(Top−Bottom)/(1+10\hat{ }((LogIC₅₀−X)*HillSlope))

X: logarithm of compound concentration; Y: Alpha Counts.

(8) Assay result:

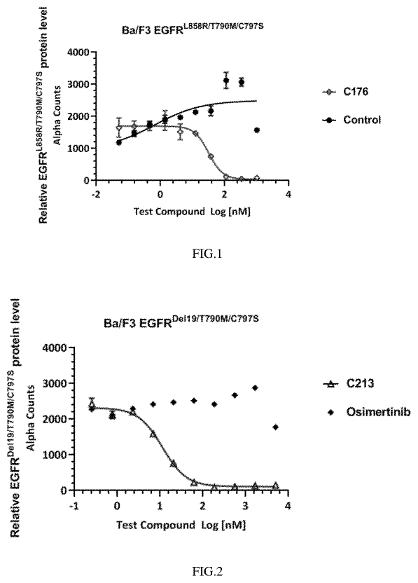

The effects of the compounds of the present disclosure on mutant EGFRL^{858R/T790M/C797S} and EGFR^{Dell9/T790M/C797S} protein levels are shown in FIGS. 1 and 2.

As shown in FIG. 1, the assay results show that in the range of 0.01 nM to 1000 nM, EGFR^{L858R/T790M/C797S} protein level decreases with the increase of the concentration of compound C176, and compound C176 significantly reduces EGFR^{58R/IT790M/C797S} protein level with a DC₅₀ of 31.37 nM, which proves that the compounds of the present disclosure have significant degradation effect on EGFR^{L858R/T790M/C797S} protein in a dose-dependent manner.

As shown in FIG. 2, the assay results show that in the range of 0.1 nM to 10000 nM, EGFR^{Dell9/T790M/C797S} protein level decreases with the increase of the concentration of compound C213, and compound C213 significantly reduces EGFR^{Dell9/T790M/C797S} protein level with a DC₅₀ of 11.28 nM, which proves that the compounds of the present disclosure have significant degradation effect on EGFR^{Dell9/T790M/C797S} protein in a dose-dependent manner.

Conclusion: the compounds of the present disclosure can significantly induce the degradation of EGFR^{Dell9/T790M/C797S} and EGFR^{58R/IT790M/C797S} proteins in cells in a dose-dependent manner.

Assay of activity on ALK kinase

1. Assay method

(1) A compound stock solution was prepared;

(2) The compound stock solution was diluted 3× to obtain a compound dilution;

(3) 100 nL of the compound dilution was transferred to ProxiPlate-384 Plus (Perkin Elmer, Perkin Elmer), in duplicates, by Echo 550;

(4) 2×ALK and ALK^{G1202R} kinase working solutions were prepared with 1× kinase buffer;

(5) 5 μl of the 2× kinase working solution was added to the reaction plate, centrifuged at 1,000 rpm for 1 min, and incubated at 25° C. for 15 min;

(6) A mixture of 2×TK-substrate-biotin (2 μM) and ATP was prepared with 1× Kinase buffer;

(7) 5 μl of the TK-substrate-biotin and ATP (the mixture prepared in step (6)) was added to initiate the reaction;

(8) The mixture was centrifuged at 1,000 g for 30 s; the plate was sealed, and incubated at 25° C. for 60 min;

(9) 4×Sa-XL 665 and TK-antibody-Cryptate were prepared with detection buffer;

(10) 5 μL of the Sa-XL 665 and 5 μL of the TK-antibody-Cryptate were added successively;

(11) The mixture was centrifuged at 1,000 g for 1 min, and incubated at 25° C. for 60 min;

(12) The fluorescence values were read at 615 nm and 665 nm by enzyme labeling instrument (PerkinElmer, 74785).

2. Data analysis

The ratio (665/615) value of each well was calculated.

Formula of inhibition rate (%):

$$Inhibition ( \% ) = [ 1 - \frac{{Ratio}_{cmpd} - {\overline{Ratio}}_{positive}}{{\overline{Ratio}}_{vehicle} - {\overline{Ratio}}_{positive}} ] \times 100$$

Ratio_{cmpd}: Ratio (665/615) value of assay compound.

R̅a̅t̅i̅o̅ₚₒₛᵢₜᵢᵥₑ: Average ratio (665/615) value of positive control.

R̅a̅t̅i̅o̅_{vehicle}: Average ratio (665/615) value of negative control.

The nonlinear regression curve (dose response-variable slope) between the value of inhibition rate (%) and the logarithm of compound concentration was fitted with graphpad prism 8.0, the effect dose curve of compound was drawn, and the IC₅₀ value was calculated.

Y=Bottom+(Top−Bottom)/(1+10\hat{ }((LogIC₅₀−X)*Hill Slope))

X-axis: logarithm of compound concentration; Y axis: inhibition rate (00 inhibition)

3. Assay result

The IC₅₀ values of the inhibitory activity of the compounds of the present disclosure on ALK and ALK^{G1202R} are shown in the table below.

| Compound | IC₅₀^{#} (nM) | |
|---|---|---|
| No. | ALK | ALK^{G1202R} |
| A1 | B | |
| A2 | A | |
| A10 | A | B |
| A11 | A | B |
| A12 | A | B |
| A13 | A | D |
| C1 | A | |
| C4 | A | B |
| C6 | A | A |
| C93 | A | A |
| C94 | A | A |
| C103 | A | D |
| C106 | A | C |
| C107 | A | B |
| C109 | A | A |
| C116 | A | C |
| C153 | A | C |
| C167 | A | C |
| C177 | A | C |
| C178 | A | C |
| C190 | A | C |
| C192 | A | C |
| C193 | A | C |
| C194 | A | D |
| Crizotinib | A | D |
| Repotrectinib | A | B |

^{#}Note:
A represents IC₅₀ of 1-100 nM, B represents IC₅₀ of 101-200 nM, C represents IC₅₀ of 201-300 nM, and D represents IC₅₀ of >300 nM.

Conclusion: the compounds of the present disclosure have strong inhibitory activity on ALK and ALKG^{1202R}.

Assay of activity on Ba/F3(EML4-ALK) cells

1. Assay method

(1) Ba/F3(EML4-ALK) cells were cultured according to the requirements of ATCC, incubated in an incubator at 37° C. and 5% CO₂, and analyzed by index; cells with viability of >90% can be used in the assay, and cells were seeded in a 384-well plate (PerkinElmer, 6007680) with 700 cells/well, 30 μl/well.

(2) A compound stock solution was prepared, and diluted 3× to obtain a compound dilution; 30 nL of the serial dilution was added to a 384-well plate (784075, Greiner) by Echo 550, and cells were incubated in an incubator at 37° C. and 5% CO₂ for 72 h.

(3) 30 μL of CTG was added to each well, and the 384-well plate was shaken on a Plate shaker (QILINBEIER, QB-9002); the 384-well plate was incubated in the dark at 37° C. and 5% CO₂ for 30 min, and the chemiluminescence value was read by Envision (PerkinElmer, EnVision 2104).

2. Data analysis

The percent inhibition rate (% inhibition) was calculated by the formula below

$$Inhibition ( \% ) = [ 1 - \frac{{LUM}_{cmpd} - {\overline{LUM}}_{positi ve}}{{\overline{LUM}}_{vehicle} - {\overline{LUM}}_{positive}} ] \times 100$$

LUM_{cmpd}: Luminescence value of assay compound.

L̅U̅M̅ₚₒₛᵢₜᵢᵥₑ: Average LUM value of positive drug at a concentration of 10 μM.

L̅U̅M̅^{vehicle}: Average LUM value of negative control group without drug treatment.

The nonlinear regression curve (dose response-variable slope) between the value of inhibition rate (%) and the logarithm of compound concentration was fitted with graphpad prism 8.0, the effect dose curve of compound was drawn, and the IC₅₀ value was calculated.

Y=Bottom+(Top−Bottom)/(1+10\hat{ }((LogIC₅₀−X)*HillSlope))

X-axis: logarithm of compound concentration; Y axis: inhibition rate (% inhibition).

3. Assay result

The IC₅₀ values of the inhibitory activity of the compounds of the present disclosure on Ba/F3(EMVL4-ALK) cells are shown in the table below.

| Compound | IC₅₀^{#} (nM) |
|---|---|
| No. | Ba/F3(EML4-ALK) |
| A10 | A |
| A11 | A |
| A12 | A |
| A13 | A |
| C1 | A |
| C47 | B |
| C116 | A |
| C148 | |
| C153 | A |
| C158 | C |
| C163 | A |
| C164 | A |
| C167 | A |
| C171 | B |
| C172 | A |
| C173 | A |
| C176 | A |
| C177 | A |
| C178 | A |
| C181 | A |
| C182 | A |
| C183 | A |
| C184 | A |
| C186 | A |
| C187 | A |
| C188 | D |
| C189 | B |
| C190 | A |
| C191 | B |
| C192 | A |
| C193 | A |
| C194 | A |
| C195 | A |
| C196 | A |
| C211 | A |
| C214 | A |
| C310 | A |
| Crizotinib | A |

^{#}Note:
A represents IC₅₀ of 1-100 nM, B represents IC₅₀ of 101-200 nM, C represents IC₅₀ of 201-300 nM, and D represents IC₅₀ of >300 nM.

Conclusion: the compounds of the present disclosure have strong inhibitory activity on Ba/F3(EML4-ALK) cells. PP28T,

The above is a further detailed description of the present disclosure in connection with the specific alternative embodiments, and the specific embodiments of the present disclosure are not limited to the description. It will be apparent to those skilled in the art that the present disclosure may be practiced by making various simple deduction and replacement, without departing from the spirit and scope of the invention.

1. A compound of general formula (X), or a pharmaceutically acceptable salt, enantiomer, diastereomer, racemate, solvate, hydrate, polymorph, prodrug, or isotopic variant thereof, or mixture thereof: 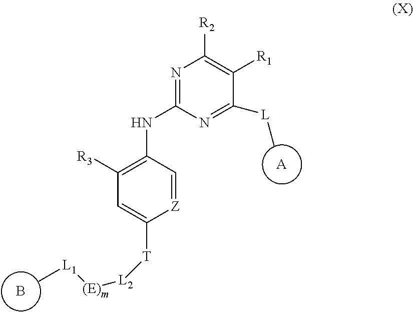
wherein
ring A is selected from the following optionally substituted groups: C₃₋₇ cycloalkyl, 4- to 8-membered heterocyclyl, C₆₋₁₀ aryl or 5- to 10-membered heteroaryl, wherein the substituent is selected from halogen, —CN, C₁₋₆ alkyl, C₁₋₆ haloalkyl, C₂₋₆ alkenyl, C₃₋₇ cycloalkyl, 4- to 8-membered heterocyclyl, —P(O)(C₁₋₆ alkyl)₂, —P(O)(C₂₋₆ alkenyl)₂, —O—C₁₋₆ alkyl, —O—C₁₋₆ haloalkyl, —O—C₂₋₆ alkenyl, —O—C₃₋₇ cycloalkyl, —O-4- to 8-membered heterocyclyl, —NH—C₁₋₆ alkyl, —NH—C₂₋₆ alkenyl, —NH—C₃₋₇ cycloalkyl, —NH-4- to 8-membered heterocyclyl, —C(O)—C₁₋₆ alkyl, —C(O)—C₂₋₆ alkenyl, —C(O)—C₃₋₇ cycloalkyl, —C(O)-4- to 8-membered heterocyclyl, —S(O)₂—C₁₋₆ alkyl, —S(O)₂—C₂₋₆ alkenyl, —S(O)₂—C₃₋₇ cycloalkyl, —S(O)₂-4- to 8-membered heterocyclyl, —NHC(O)—C₁₋₆ alkyl, —NHC(O)—C₂₋₆ alkenyl, —NHC(O)—C₃₋₇ cycloalkyl, —NHC(O)-4- to 8-membered heterocyclyl, —NHS(O)₂—C₁₋₆ alkyl, —NHS(O)₂—C₂₋₆ alkenyl, —NHS(O)₂—C₃₋₇ cycloalkyl or —NHS(O)₂-4- to 8-membered heterocyclyl;
ring B is selected from the following group: 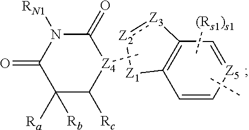 [symbol]
represents single bond or double bond; [symbol]
represents that the point of attachment to the rest of the molecule can be located at the available point of the ring;
Z₁ is O, S, N or C atom, which is optionally substituted with one or two R_{Z1}; or Z₁ is absent, and thus Z₄ is connected to Z₂, Z₃ or the C atom connected to Z₁ on the aromatic ring, and the Z₂ and the C atom on the aromatic ring that are connected to Z₁ are connected to R_{W} respectively; or Z₁, Z₂ and Z₃ are all absent, and thus Z₄ is connected to one of the C atoms connected to Z₁ or Z₃ on the aromatic ring, and the other C atom on the aromatic ring is connected to R_{W};
Z₂ is O, S, N or C atom, which is optionally substituted with one or two R_{Z2};
Z₃ is O, S, N or C atom, which is optionally substituted with one or two R_{Z3}; with the proviso that when [symbol] represents double bond, Z₂ is N or C atom, and Z₃ is N or C atom;
Z₄ is N or CR_{Z4};
Z₅ is N or CR_{Z5};
R^{a}, R^{b} and R_{c} are independently H, halogen, —(CH₂)₀₋₅—OR″, —(CH₂)₀₋₅—NR″R″′, C₁₋₆ alkyl or C₁₋₆ haloalkyl; or R^{a} and R^{b} are taken together with the carbon atom to which they are attached to form C═O, C₃₋₇ cycloalkyl or 4- to 8-membered heterocyclyl; or R^{a} and R, are taken together with the carbon atoms to which they are attached to form C₃₋₇ cycloalkyl or 4- to 8-membered heterocyclyl; or R^{a} and R, are taken together to form bond;
R_{N1} is H, C₁₋₆ alkyl or C₁₋₆ haloalkyl, alternatively H;
R_{Z1} is absent, H, CN, halogen, —(CH₂)₀₋₅—OR″, —(CH₂)₀₋₅—NR″R″′, C₁₋₆ alkyl, C₁₋₆ haloalkyl, —(CH₂)₀₋₅—C₃₋₇ cycloalkyl or —(CH₂)₀₋₅-4- to 8-membered heterocyclyl; or two R_{Z1} are taken together with Z₁ to form C═O, C₃₋₇ cycloalkyl or 4- to 8-membered heterocyclyl;
R_{Z2} is absent, H, CN, halogen, —(CH₂)₀₋₅—OR″, —(CH₂)₀₋₅—NR″R″′, C₁₋₆ alkyl, C₁₋₆ haloalkyl, —(CH₂)₀₋₅—C₃₋₇ cycloalkyl or —(CH₂)₀₋₅-4- to 8-membered heterocyclyl; or two R_{Z2} are taken together with Z₂ to form C═O, C₃₋₇ cycloalkyl or 4- to 8-membered heterocyclyl;
R_{Z3} is absent, H, CN, halogen, —(CH₂)₀₋₅—OR″, —(CH₂)₀₋₅—NR″R″′, C₁₋₆ alkyl, C₁₋₆ haloalkyl, -(CH₂)₀₋₅—C₃₋₇ cycloalkyl or —(CH₂)₀₋₅-4- to 8-membered heterocyclyl; or two R_{Z3} are taken together with Z₃ to form C═O, C₃₋₇ cycloalkyl or 4- to 8-membered heterocyclyl;
R_{Z4} is H, CN, halogen, —(CH₂)₀₋₅—OR″, —(CH₂)₀₋₅—NR″R″′, C₁₋₆ alkyl or C₁₋₆ haloalkyl;
R_{Z5} is H, CN, halogen, —(CH₂)₀₋₅—OR″, —(CH₂)₀₋₅—NR″R″′, C₁₋₆ alkyl, C₁₋₆ haloalkyl, —(CH₂)₀₋₅—C₃₋₇ cycloalkyl or —(CH₂)₀₋₅-4- to 8-membered heterocyclyl;
or the ring in which Z₄ is located is absent;
wherein R_{W} is H, CN, halogen, —(CH₂)₀₋₅—OR″, —(CH₂)₀₋₅—NR″R″′, —C₁₋₆ alkyl, C₁₋₆ haloalkyl, —(CH₂)₀₋₅—C₃₋₇ cycloalkyl, —(CH₂)₀₋₅-4- to 8-membered heterocyclyl, C₂₋₆ alkenyl, C₂₋₆ alkynyl, —(CH₂)₀₋₅—C₃₋₁₀ halocycloalkyl, —(CH₂)₀₋₅—C₆₋₁₀ aryl or —(CH₂)₀₋₅-5- to 14-membered heteroaryl;
R″ is H, C₁₋₆ alkyl, C₁₋₆ haloalkyl or —(CH₂)₀₋₅—C₃₋₇ cycloalkyl;
R″′ is H, C₁₋₆ alkyl or C₁₋₆ haloalkyl;
L₁ is selected from bond, —O—, —S(O)ₚ—, —S(O)(═NR*)—, —NR^{#}—, —CR^{#}R^{#}′—, —CₐR^{#}R^{#′—C}_{b}R^{#}R^{#}′—, —N═S(O)(R*)— or —S(O)(R*)═N—;
L₂ is selected from bond, —O—, —S(O)ₚ—, —S(O)(═NR*)—, —NR^{#}—, —CR^{#}R^{#}′—, —CₐR^{#}R^{#′—C}_{b}R^{#}R^{#}′—, —N═S(O)(R*)— or —S(O)(R*)═N—;
wherein one of CₐR^{#}R^{#}′ or C_{b}R^{#}R^{#}′ can be replaced by O, S(O)ₚ, S(O)(═NR*) or NR^{#}, and when one of CₐR^{#}R^{#}′ or C_{b}R^{#}R′ is replaced by O, S or NR^{#}, the other of CₐR^{#}R^{#}′ or C_{b}R^{#}R^{#}′ can also be replaced by S(O)_{q};
E is independently selected from: bond, —C_{c}R^{#}R^{#}′—C_{d}R^{#}R^{#}′—C_{c}R^{#}′, 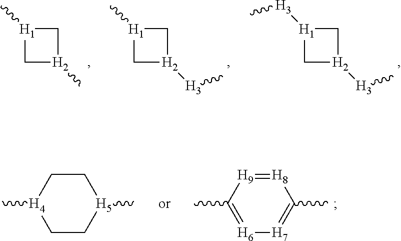
wherein one of C_{c}R^{#}R^{#}′, C_{d}R^{#}R^{#}′ or CₑR^{#}R^{#}′, or both of C_{c}R^{#}R′ and CₑR^{#}R^{#}′ can be replaced by O, S(O)ₚ, S(O)(═NR*) or NR^{#}, and when one of C_{c}R^{#}R′, C_{d}R^{#}R^{#}′ or CₑR^{#}R^{#}′ is replaced by O, S or NR^{#}, the other adjacent one or two of C_{c}R^{#}R^{#}′, C_{d}R^{#}R^{#}′ or CₑR^{#}R′ can also be replaced by S(O)_{q};
or two E moieties can be taken together to form —CH₂CH₂OCH₂CH₂—, —OCH₂CH₂CH₂CH₂—, —CH₂CH₂CH₂CH₂O—, 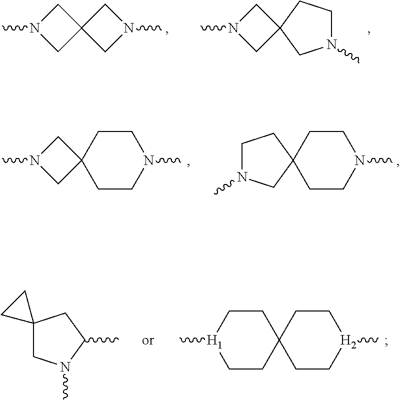
wherein [symbol] represents the point of attachment to L₁ or L₂;
H₁ and H₂ are N or C atom, H₃ is O, S, N or C atom, and H₁ and H₃, and H₂ and H₃ are not heteroatoms at the same time;
H₄ and H₅ are N or C atom;
H₆, H₇, H₈ and H₉ are C or N atom;
p is 0, 1 or 2;
q is 1 or 2;
R* is H, C₁₋₆ alkyl, C₁₋₆ haloalkyl, C₂₋₆ alkenyl, C₂₋₆ alkynyl, C₃₋₁₀ cycloalkyl, C₃₋₁₀ halocycloalkyl, 3- to 10-membered heterocyclyl, C₆₋₁₀ aryl or 5- to 14-membered heteroaryl;
R^{#} is H, halogen, C₁₋₆ alkyl, C₁₋₆ haloalkyl, C₂₋₆ alkenyl, C₂₋₆ alkynyl, C₃₋₁₀ cycloalkyl, C₃₋₁₀ halocycloalkyl, 3- to 10-membered heterocyclyl, C₆₋₁₀ aryl or 5- to 14-membered heteroaryl;
R^{#}′ is H, halogen, C₁₋₆ alkyl, C₁₋₆ haloalkyl, C₂₋₆ alkenyl, C₂₋₆ alkynyl, C₃₋₁₀ cycloalkyl, C₃₋₁₀ halocycloalkyl, 3- to 10-membered heterocyclyl, C₆₋₁₀ aryl or 5- to 14-membered heteroaryl;
or, R^{#} and R^{#} on adjacent atoms can be taken together to form bond, and R^{#}′ and R^{#}′ on adjacent atoms can be taken together to form bond;
or, R^{#} and R^{#}′ on the same or different atoms can be taken together to form ═O, or C₃₋₇ cycloalkyl, 4- to 8-membered heterocyclyl, C₆₋₁₀ aryl or 5- to 6-membered heteroaryl, wherein the C₃₋₇ cycloalkyl, 4- to 8-membered heterocyclyl, C₆₋₁₀ aryl or 5- to 6-membered heteroaryl is optionally substituted with Rₓ, wherein the Rₓ is H, CN, halogen, C₁₋₆ alkyl or C₁₋₆ haloalkyl;
m is 0, 1, 2, 3, 4, 5, 6, 7, 8, 9 or 10;
Rₛ₁ is selected from H, CN, halogen, —(CH₂)₀₋₅—OR″, —(CH₂)₀₋₅—NR″R″′, C₁₋₆ alkyl, C₁₋₆ haloalkyl, —(CH₂)₀₋₅-4- to 8-membered heterocyclyl, C₂₋₆ alkenyl, C₂₋₆ alkynyl, —(CH₂)₀₋₅—C₃₋₇ cycloalkyl, —(CH₂)₀₋₅—C₃₋₁₀ halocycloalkyl, —(CH₂)₀₋₅—C₆₋₁₀ aryl, —(CH₂)₀₋₅-5- to 14-membered heteroaryl, —C(O)R_{W}, —S(O)R_{W} or —S(O)₂R_{W};
s1 is 0, 1, 2 or 3;
R′ is selected from H, —C(O)—C₁₋₆ alkyl, —C(O)—C₁₋₆ haloalkyl, —C(O)—C₂₋₆ alkenyl or —C(O)—C₆₋₁₀ aryl;
L is bond, —O— or —NR—;
wherein R is H or C₁₋₆ alkyl;
Z is —N═ or —C(R^{#})═;
T is selected from bond, C₂₋₆ heteroalkylene, 4- to 12-membered heterocyclylene, or 5- to 6-membered heterocyclylene, wherein the 5- to 6-membered heterocyclylene is substituted with 5- to 6-membered heterocyclylene;
R₁ is selected from H, halogen, cyano, C₁₋₆ alkyl, C₃₋₇ cycloalkyl, 4- to 8-membered heterocyclyl, C₁₋₆ haloalkyl, C₂₋₆ alkenyl or C₂₋₆ alkynyl;
or, when L is —NR—, R₁ and R are taken together with the atoms to which they are attached to form optionally substituted 5- to 6-membered heterocyclyl, wherein the substituent is selected from halogen, oxo, C₁₋₆ alkyl, C₁₋₆ haloalkyl, C₆₋₁₀ aryl, wherein the C₆₋₁₀ aryl is mono- or poly-substituted with halogen;
R₂ is H, halogen, hydroxyl, amino, C₁₋₆ alkyl or C₁₋₆ haloalkyl;
or R₁ and R₂ are taken together with the atoms to which they are attached to form 5- to 6-membered heterocyclyl or 5- to 6-membered heteroaryl;
R₃ is selected from H, —O—C₁₋₆ alkyl or —O—C₁₋₆ haloalkyl;
R₄ is selected from H, halogen, C₁₋₆ alkyl, C₁₋₆ haloalkyl, —NHC(O)—C₁₋₆ alkyl or —NHC(O)—C₂₋₆ alkenyl;
if the above groups are H or H-containing groups, the one or more H atom(s) may be substituted with D atom(s);
the groups containing OH, NH, NH₂, CH, CH₂, or CH₃ in L₁, E, L₂, and T, or the above alkyl, alkylene, haloalkyl, alkenyl, alkynyl, cycloalkyl, halocycloalkyl, heterocyclyl, aryl, and heteroaryl are each optionally substituted with 1, 2, 3 or more R^{s} or isotopic variants thereof at each occurrence, wherein the R^{s} is independently selected from the following groups at each occurrence: halogen, hydroxyl, amino, cyano, nitro, C₁₋₆ alkyl, C₁₋₆ haloalkyl, C₂₋₆ alkenyl, C₂₋₆ alkynyl, C₃₋₁₀ cycloalkyl, C₃₋₁₀ halocycloalkyl, 3- to 10-membered heterocyclyl, C₆₋₁₀ aryl, 5- to 14-membered heteroaryl, C₆₋₁₂ aralkyl, —OR^{a′}, —OC(O)R^{a′}, —C(O)R^{a′}, —C(O)OR^{a′}, —C(O)NR^{a′}R^{b′}, —S(O)R^{a′}, —S(O)ₙOR^{a′}, —S(O)ₙNR^{a′}R^{b′}, —NR^{a′}R^{b′}, —NR^{a′}C(O)R^{b′}, —NR^{a′}—C(O)OR^{b′}, —N_{a′}S(O)ₙ—R^{b′}, —NR^{a′}C(O)NR^{a′}R′, —C₁₋₆ alkylene-R^{a′}, —C₁₋₆ alkylene-OR^{a′}, —C₁₋₆ alkylene-OC(O)R^{a′}, —C₁₋₆ alkylene-C(O)OR^{a′}, —C₁₋₆ alkylene-S(O)ₙR^{a′}, —C₁₋₆ alkylene-S(O)ₙOR^{a′}, —C₁₋₆ alkylene-OC(O)NR^{a′}R′, —C₁₋₆ alkylene-C(O)NR^{a′}R′, —C₁₋₆ alkylene-NR^{a′}—C(O)NR^{a′}R^{b′}, —C₁₋₆ alkylene-OS(O)ₙR^{a}, —C₁₋₆ alkylene-S(O)ₙNR^{a′}R′, —C₁₋₆ alkylene-NR^{a′}—S(O)ₙNR^{a′}R′, —C₁₋₆ alkylene-NR^{a′}R′ and —O—C₁₋₆ alkylene-NR^{a′}R′, and wherein the hydroxyl, amino, alkyl, alkylene, cycloalkyl, heterocyclyl, aryl, heteroaryl and aralkyl described with respect to the substituent R^{s} are further optionally substituted with 1, 2, 3 or more substituents independently selected from the following groups or an isotopic variant thereof: halogen, OH, amino, cyano, nitro, C₁₋₆ alkyl, C₁₋₆ haloalkyl, C₁₋₆ alkyl hydroxyl, C₃₋₆ cycloalkyl, 3- to 10-membered heterocyclyl, C₆₋₁₀ aryl, 5- to 14-membered heteroaryl and C₆₋₁₂ aralkyl;
n is independently 1 or 2 at each occurrence;
each of R^{a′} and R^{b′} is independently selected from H, C₁₋₆ alkyl, C₂₋₆ alkenyl, C₂₋₆ alkynyl, C₁₋₆ alkyl-O—, C₁₋₆ alkyl-S—, C₃₋₁₀ cycloalkyl, 3- to 10-membered heterocyclyl, C₆₋₁₀ aryl, 5- to 14-membered heteroaryl and C₆₋₁₂ aralkyl at each occurrence.

2. The compound of general formula (X), or the pharmaceutically acceptable salt, enantiomer, diastereomer, racemate, solvate, hydrate, polymorph, prodrug, or isotopic variant thereof, or mixture thereof according to claim 1, which has the structure of general formula (I): 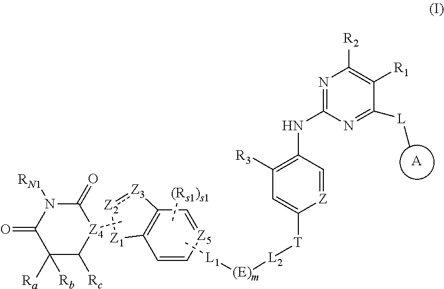
wherein each group is as defined in claim 1.

3. The compound of general formula (I), or the pharmaceutically acceptable salt, enantiomer, diastereomer, racemate, solvate, hydrate, polymorph, prodrug, or isotopic variant thereof, or mixture thereof according to claim 2, wherein
ring A is the following groups: 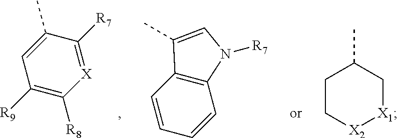
wherein
R₇ is selected from C₁₋₆ alkyl, C₁₋₆ haloalkyl, C₂₋₆ alkenyl, C₃₋₇ cycloalkyl, —P(O)(C₁₋₆ alkyl)₂ or —P(O)(C₂₋₆ alkenyl)₂;
R₈ is selected from H, —NH—C₁₋₆ alkyl, —NH—C₂₋₆ alkenyl, —NH—C₃₋₇ cycloalkyl, —NH-4- to 8-membered heterocyclyl, —NHC(O)—C₁₋₆ alkyl, —NHC(O)—C₂₋₆ alkenyl, —NHC(O)—C₃₋₇ cycloalkyl, —NHC(O)-4- to 8-membered heterocyclyl, —NHS(O)₂—C₁₋₆ alkyl or —NHS(O)₂—C₃₋₇ cycloalkyl;
R₉ is selected from H, halogen, C₁₋₆ alkyl, —CN, C₁₋₆ haloalkyl, —O—C₁₋₆ alkyl, —O—C₁₋₆ haloalkyl, C₃₋₇ cycloalkyl, —O—C₃₋₇ cycloalkyl, —NH—C₁₋₆ alkyl, —NH—C₂₋₆ alkenyl, —NH—C₃₋₇ cycloalkyl, —NH-4- to 8-membered heterocyclyl, —NHC(O)—C₁₋₆ alkyl, —NHC(O)—C₂₋₆ alkenyl, —NHC(O)—C₃₋₇ cycloalkyl, —NHC(O)-4- to 8-membered heterocyclyl, —NHS(O)₂—C₁₋₆ alkyl or —NHS(O)₂—C₃₋₇ cycloalkyl;
X is —C(Rₓ)═ or —N═;
wherein Rₓ is H, or Rₓ and Rₛ are taken together with the C atoms to which they are attached to form 5- to 6-membered heterocyclyl or 5- to 6-membered heteroaryl; alternatively, Rₓ and Rₛ are taken together with the C atoms to which they are attached to form 5- to 6-membered heteroaryl, alternatively pyrazinyl;
X₁ is —CH(R_{X1})— or —N(R_{X1})—;
X₂ is —CH(R_{X2})— or —N(R_{X2})—;
wherein R_{X1} is selected from H, C₁₋₆ alkyl, C₃₋₇ cycloalkyl, —O—C₁₋₆ alkyl, —O—C₃₋₇ cycloalkyl, —NH—C₁₋₆ alkyl, —NH—C₃₋₇ cycloalkyl, —S(O)₂—C₁₋₆ alkyl, —S(O)₂—C₃₋₇ cycloalkyl, —NHS(O)₂—C₁₋₆ alkyl, —NHS(O)₂—C₃₋₇ cycloalkyl, —C(O)—C₁₋₆ alkyl, —C(O)—C₂₋₆ alkenyl, —C(O)—C₃₋₇ cycloalkyl, —NHC(O)—C₁₋₆ alkyl, —NHC(O)—C₃₋₇ cycloalkyl or —C(O)-4- to 8-membered heterocyclyl; R_{X2} is selected from H, C₁₋₆ alkyl, C₃₋₇ cycloalkyl, —O—C₁₋₆ alkyl, —O—C₃₋₇ cycloalkyl, —NH—C₁₋₆ alkyl, —NH—C₃₋₇ cycloalkyl, —C(O)—C₁₋₆ alkyl, —C(O)—C₃₋₇ cycloalkyl, —C(O)-4- to 8-membered heterocyclyl, —S(O)₂—C₁₋₆ alkyl, —S(O)₂—C₃₋₇ cycloalkyl, —NHS(O)₂—C₁₋₆ alkyl, —NHS(O)₂—C₃₋₇ cycloalkyl, —NHC(O)—C₁₋₆ alkyl, —NHC(O)—C₂₋₆ alkenyl, —NHC(O)—C₃₋₇ cycloalkyl or —NHC(O)— 4- to 8-membered heterocyclyl; [symbol]
represents the point of attachment to L.

4. The compound of general formula (I), or the pharmaceutically acceptable salt, enantiomer, diastereomer, racemate, solvate, hydrate, polymorph, prodrug, or isotopic variant thereof, or mixture thereof according to claim 2, wherein
T is
bond, 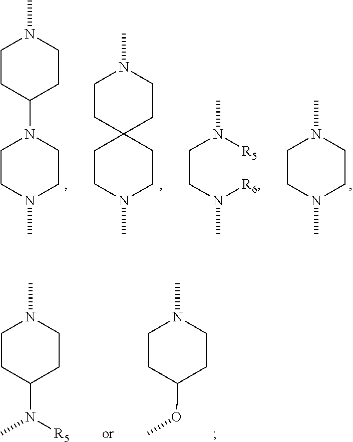
wherein
R₅ is H or C₁₋₆ alkyl;
R₆ is H or C₁₋₆ alkyl;
or R₅ and R⁶ are connected to form C₁₋₆ alkylene; [symbol]
represents the point of attachment to parent core or L₂.

5. The compound of general formula (I), or the pharmaceutically acceptable salt, enantiomer, diastereomer, racemate, solvate, hydrate, polymorph, prodrug, or isotopic variant thereof, or mixture thereof according to claim 2, wherein when L is —NR—, R₁ and R are taken together to form the following groups: —C(O)N(R_{N})C(O)— or —C(C₁₋₆ alkyl)═C(R_{N})C(O)—;
wherein
R_{N} is selected from C₁₋₆ alkyl or 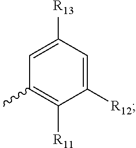
R₁₁ is H or halogen;
R¹² is H or halogen;
R₁₃ is H or halogen; [symbol]
represents the point of attachment.

6. The compound of general formula (I), or the pharmaceutically acceptable salt, enantiomer, diastereomer, racemate, solvate, hydrate, polymorph, prodrug, or isotopic variant thereof, or mixture thereof according to claim 2, wherein R₁ and R₂ are taken together with the atoms to which they are attached to form 5- to 6-membered heteroaryl; alternatively form pyrrolyl.

7. The compound of general formula (I), or the pharmaceutically acceptable salt, enantiomer, diastereomer, racemate, solvate, hydrate, polymorph, prodrug, or isotopic variant thereof, or mixture thereof according to claim 2, wherein
ring A is selected from the following optionally substituted groups: C₃₋₇ cycloalkyl, 4- to 8-membered heterocyclyl, C₆₋₁₀ aryl or 5- to 10-membered heteroaryl, wherein the substituent is selected from —F, —Cl, —Br, -Me, —OMe, —CF₃, —OCF₃, —CN, —NHMe, cyclopropyl, —P(O)Me₂, —NHC(O)CH₂CH₃, —C(O)CH═CH₂, —NHS(O)₂CH₂CH₃, —NH-cyclopropyl, —NHC(O)CH═CH₂ or —C(O)CH₂CH₃; ring A is alternatively the following groups: 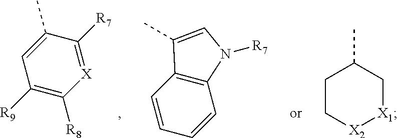
wherein
R₇ is selected from -Me, cyclopropyl or —P(O)Me₂;
R₈ is selected from H, —NHMe, —NHC(O)CH₂CH₃ or —NH-cyclopropyl;
R₉ is selected from H, —F, —Cl, —Br, -Me, —CF₃, —OMe, —OCF₃, —CN, —NHC(O)CH₂CH₃, —NHS(O)₂CH₂CH₃, —NHC(O)CH═CH₂ or —NH-cyclopropyl;
X is —C(Rₓ)═ or —N═;
wherein Rₓ is H, or Rₓ and Rₛ are taken together with the C atoms to which they are attached to form 5- to 6-membered heterocyclyl or 5- to 6-membered heteroaryl; alternatively 5- to 6-membered heteroaryl (alternatively pyrazinyl);
X₁ is —CH₂— or —N(R_{X1})—;
X₂ is —CH(R_{X2})— or —N(R_{X2})—;
wherein R_{X1} is —C(O)CH₂CH₃ or —C(O)CH═CH₂; R_{X2} is H, -Me, —OMe, —NHMe, —C(O)CH₂CH₃, —NHS(O)₂CH₂CH₃ or —NHC(O)CH₂CH₃; [symbol]
represents the point of attachment to L; [symbol]
T is selected from bond, C₂₋₆ heteroalkylene, 4- to 12-membered heterocyclylene, or 5- to 6-membered heterocyclylene, wherein the 5- to 6-membered heterocyclylene is substituted with 5- to 6-membered heterocyclylene; or is alternatively the following groups: 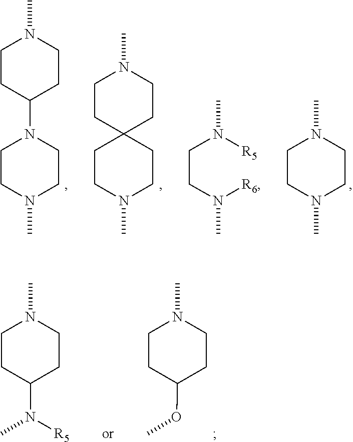
wherein
R₅ is -Me;
R₆ is -Me;
or R₅ and R⁶ are connected to form —CH₂CH₂—; [symbol]
represents the point of attachment to parent core or L₂;
R₁ is selected from H, —Cl, —Br, —CH₃, CF₃, cyclopropyl or —CH═CH₂;
or, when L is —NR—, R₁ and R are taken together with the atoms to which they are attached to form optionally substituted 5-to 6-membered heterocyclyl, wherein the substituent is selected from halogen, oxo, -iPr, -Et, or phenyl, wherein the phenyl is mono- or poly-substituted with halogen; R₁ and R are alternatively taken together to form the following groups: —C(O)N(R_{N})C(O)— or —C(CH₃)═C(R_{N})C(O)—;
wherein
R_{N} is selected from -iPr, -Et or 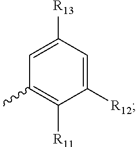
R₁₁ is —Cl or —Br;
R₁₂ is H or —F;
R₁₃ is H or —F; [symbol]
represents the point of attachment;
R₂ is H;
or R₁ and R₂ are taken together with the atoms to which they are attached to form 5- to 6-membered heterocyclyl or 5- to 6-membered heteroaryl; alternatively form pyrrolyl;
R₃ is selected from H or —OMe;
R₄ is selected from H, —F, -Me, —CF₃ or —NHC(O)CH═CH₂;
other groups are as defined in claim 2.

8. The compound of general formula (I), or the pharmaceutically acceptable salt, enantiomer, diastereomer, racemate, solvate, hydrate, polymorph, prodrug, or isotopic variant thereof, or mixture thereof according to claim 2, wherein the compound of general formula (I) has the structure of the following general formulas: 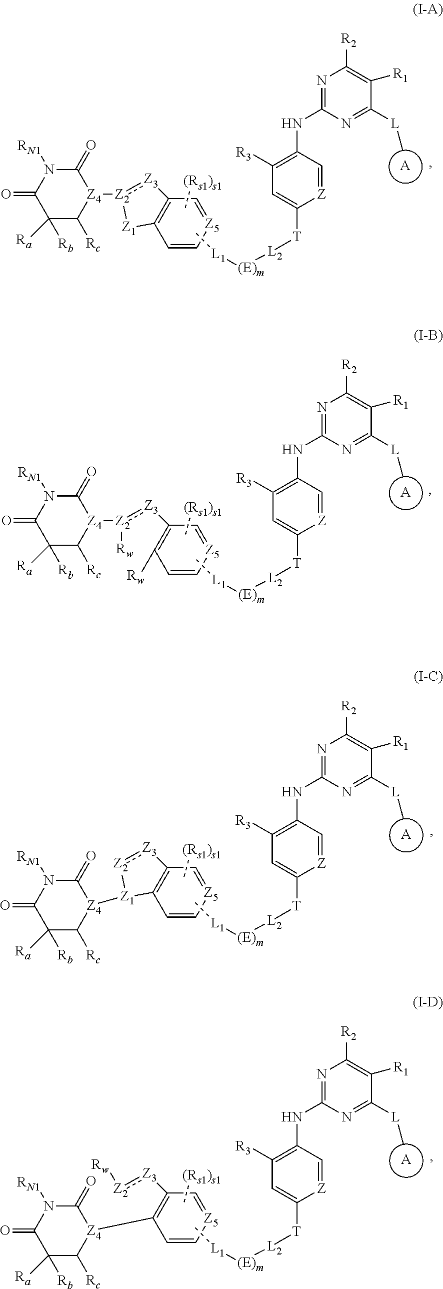 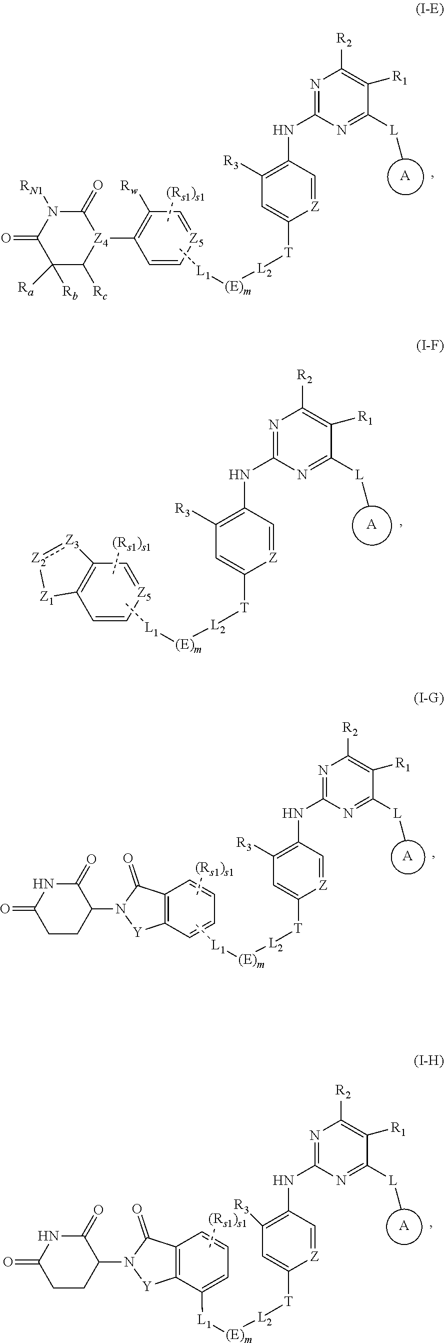 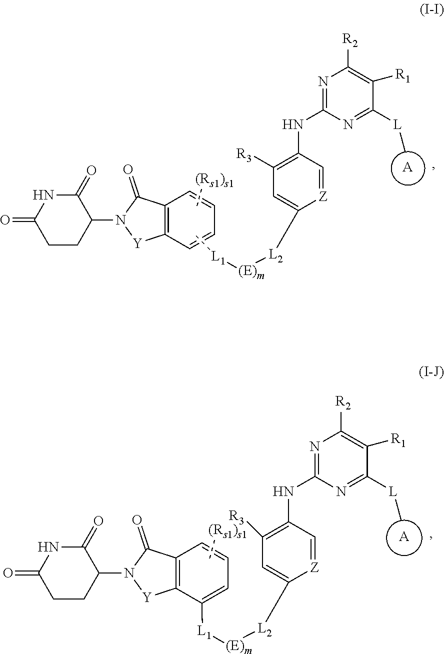 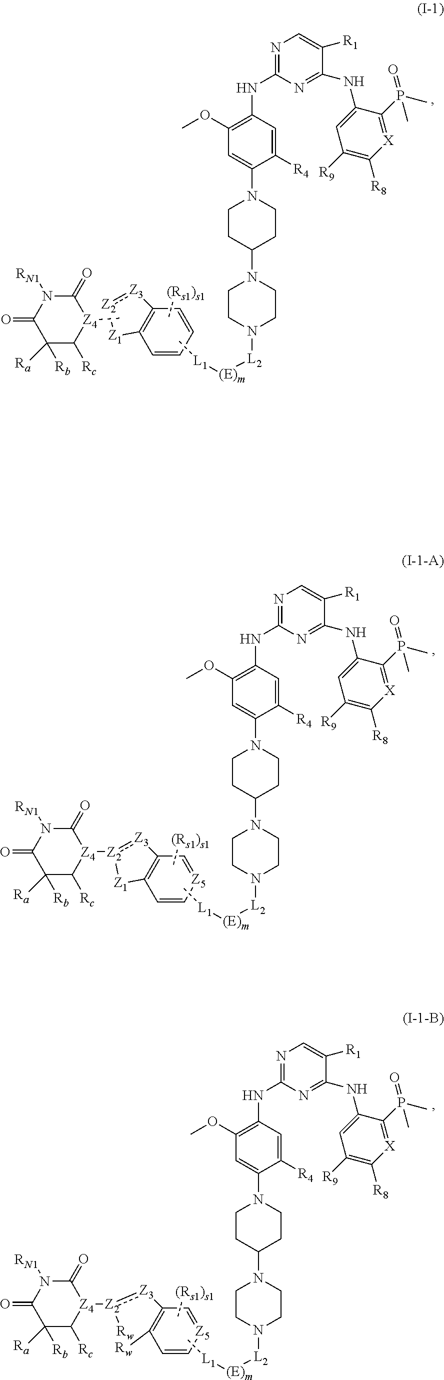 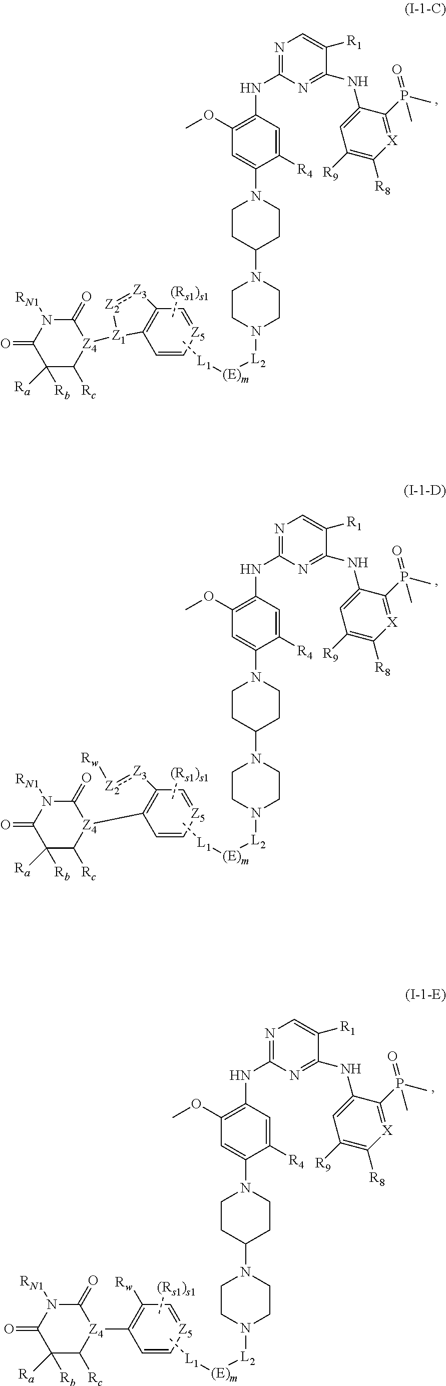 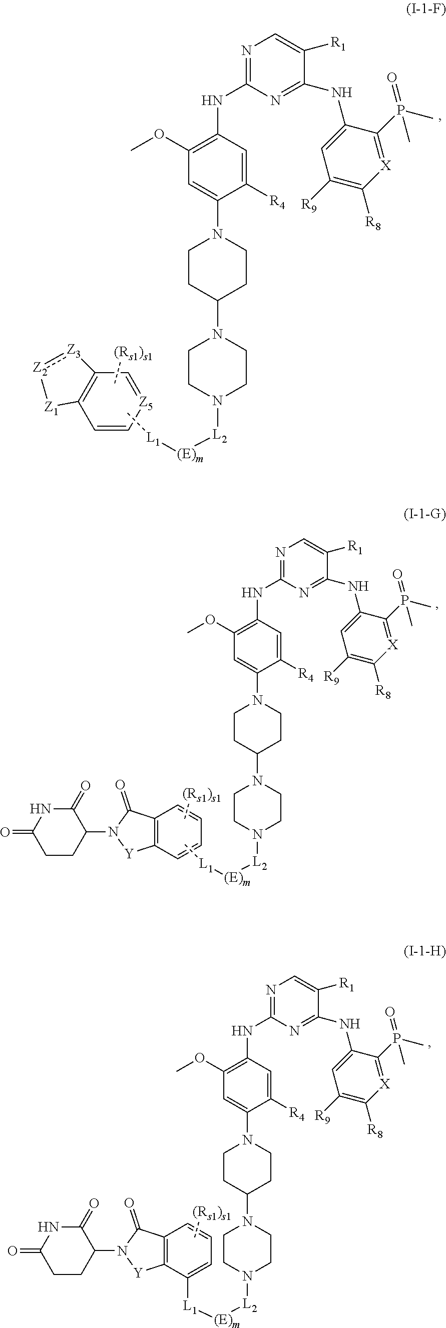 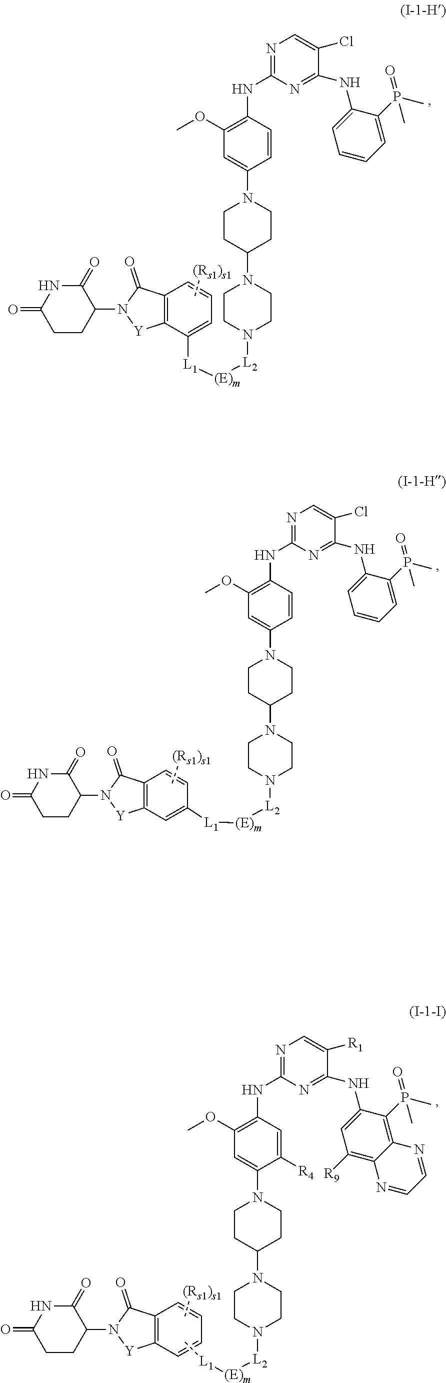 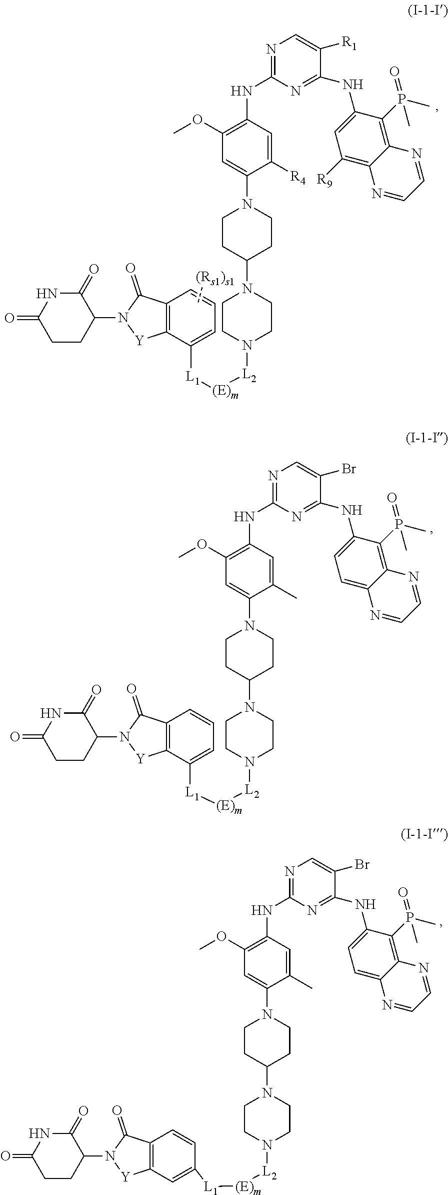 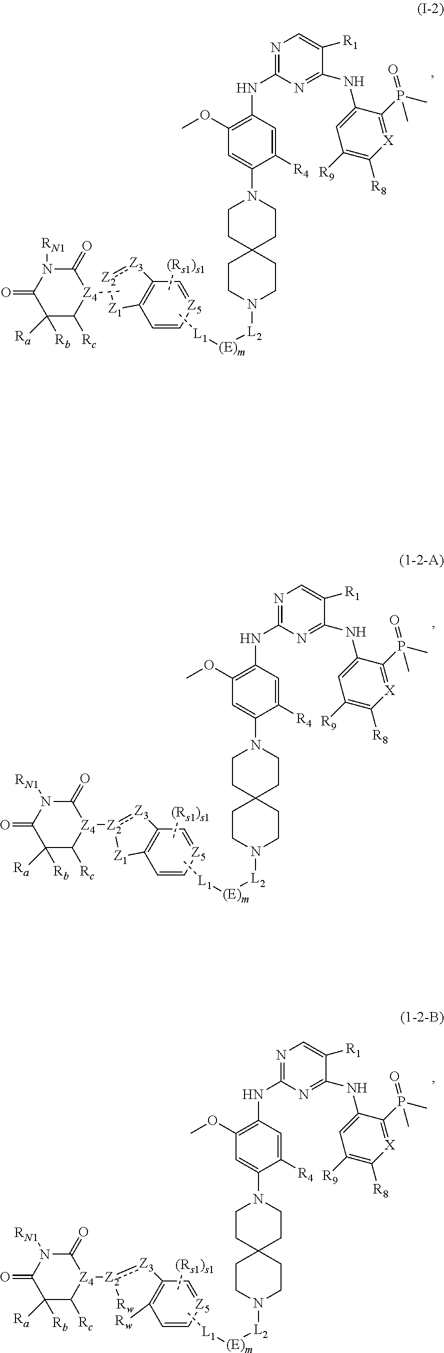 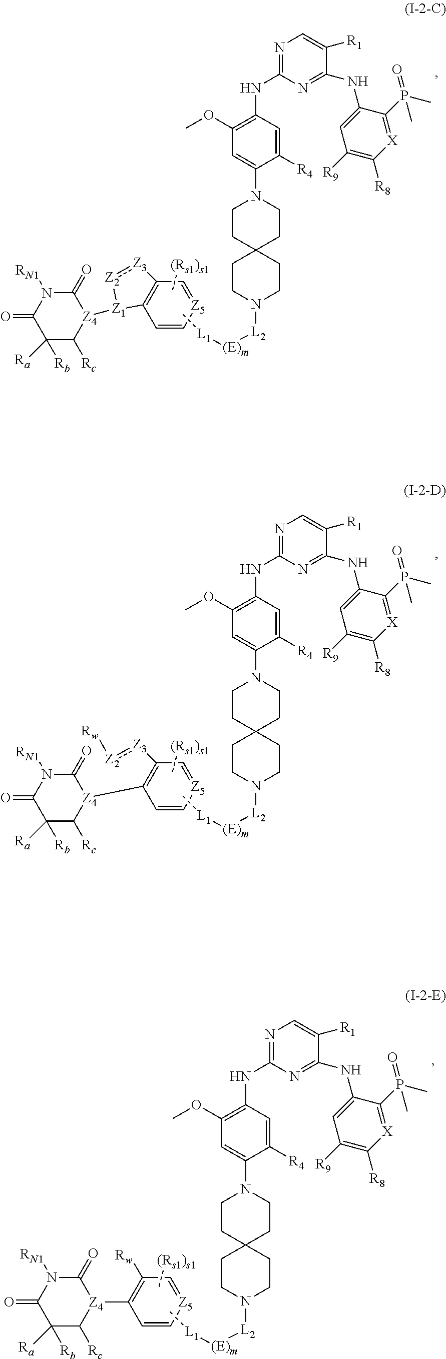 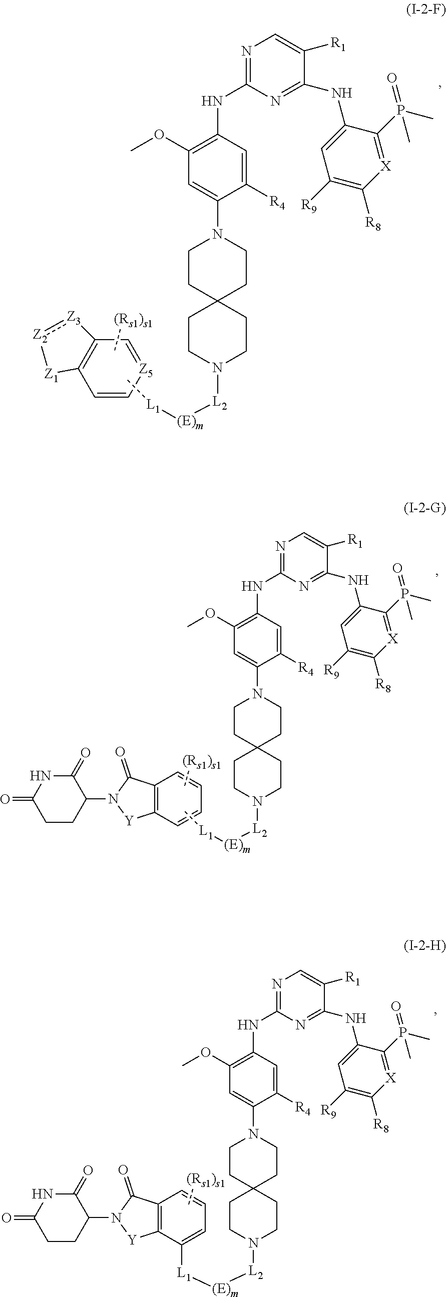 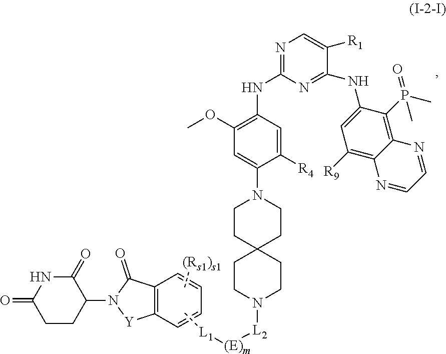 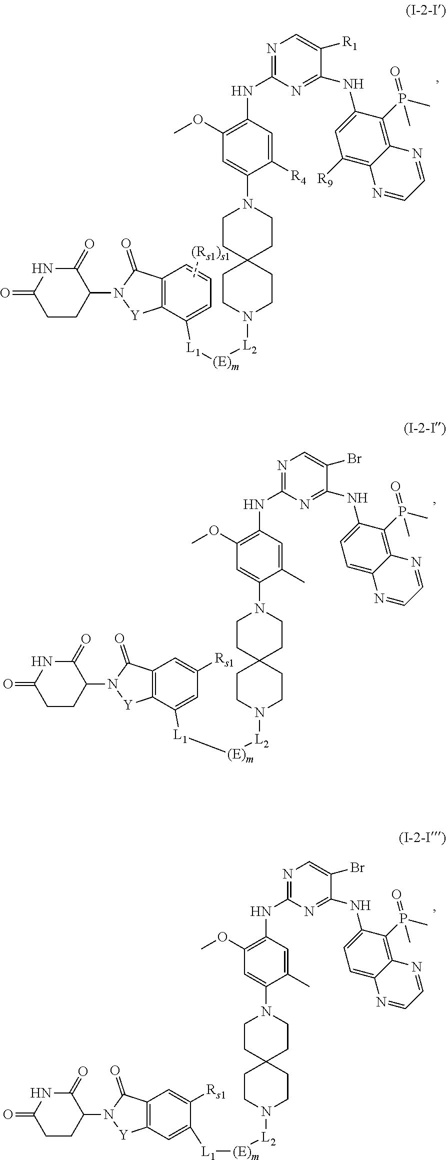 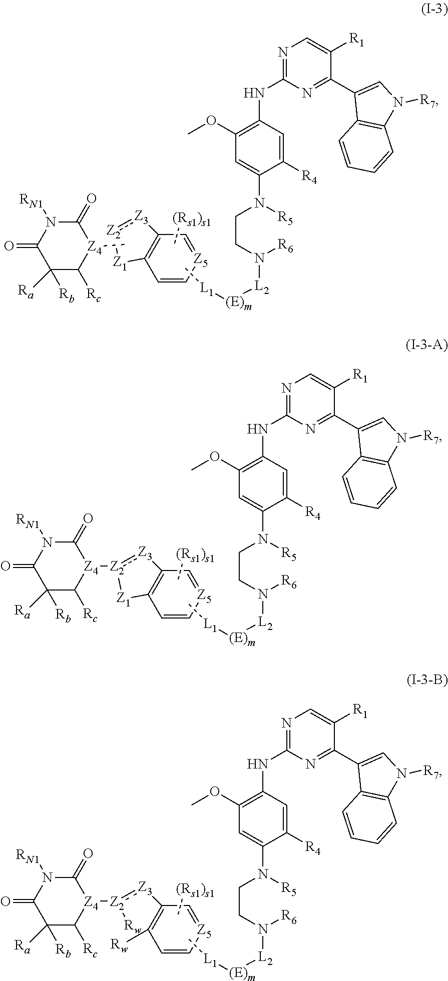 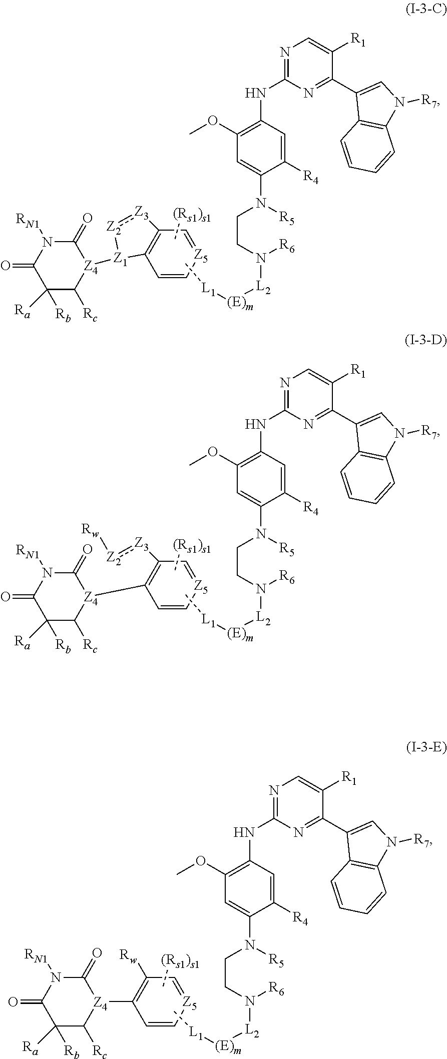 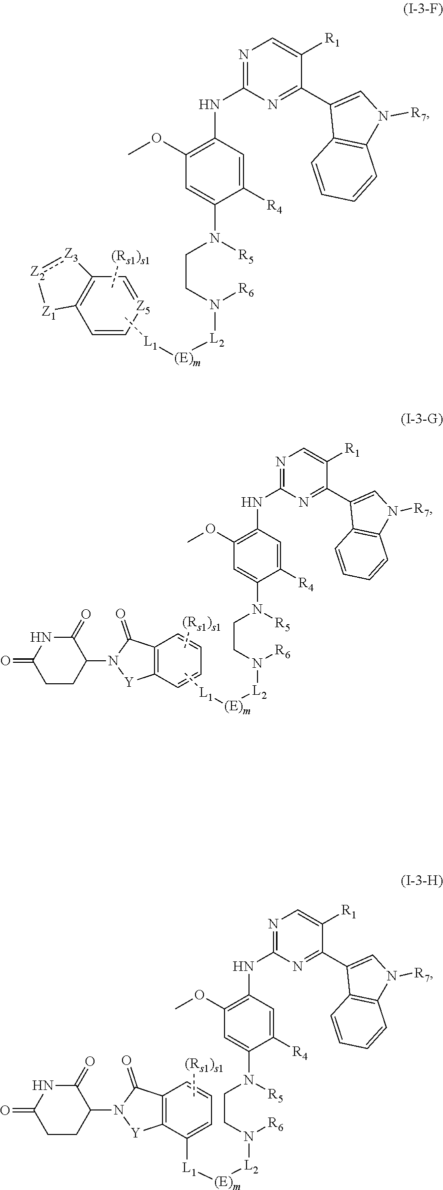 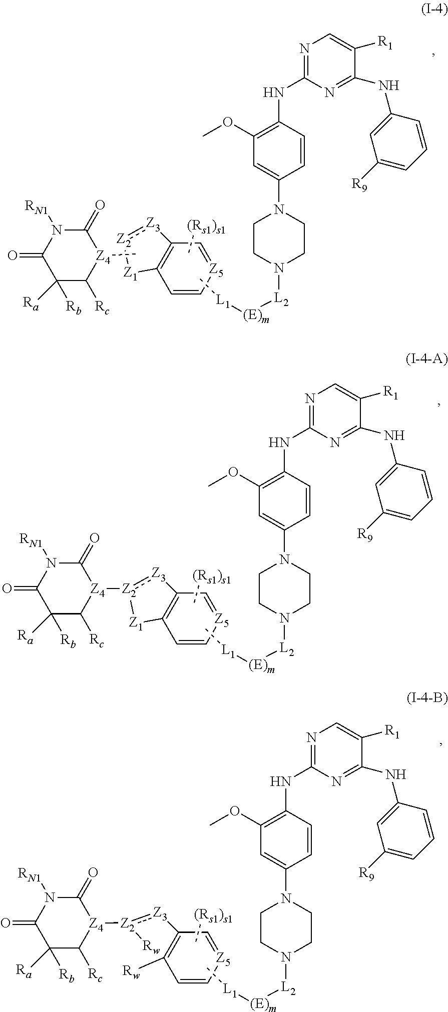 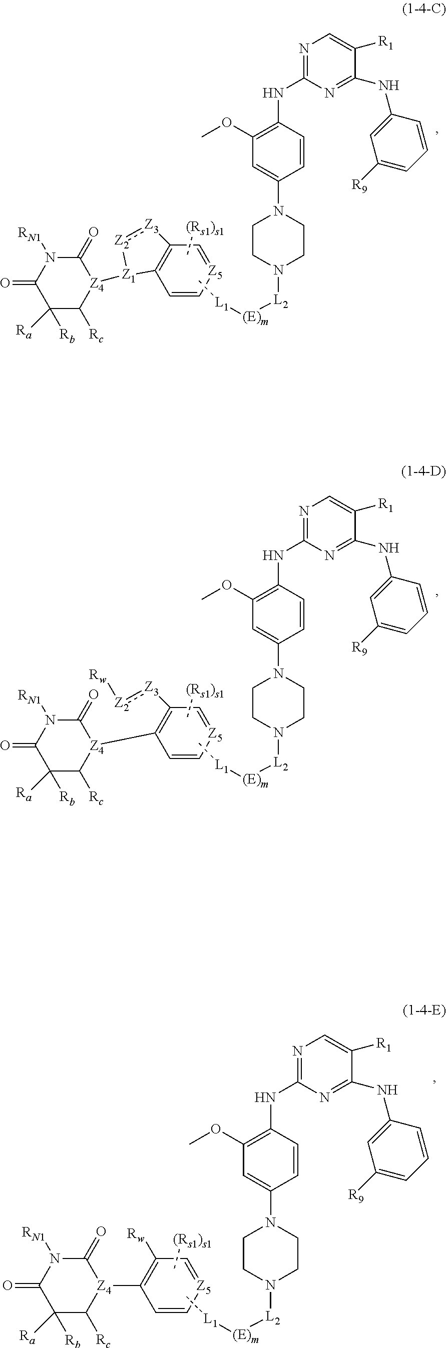 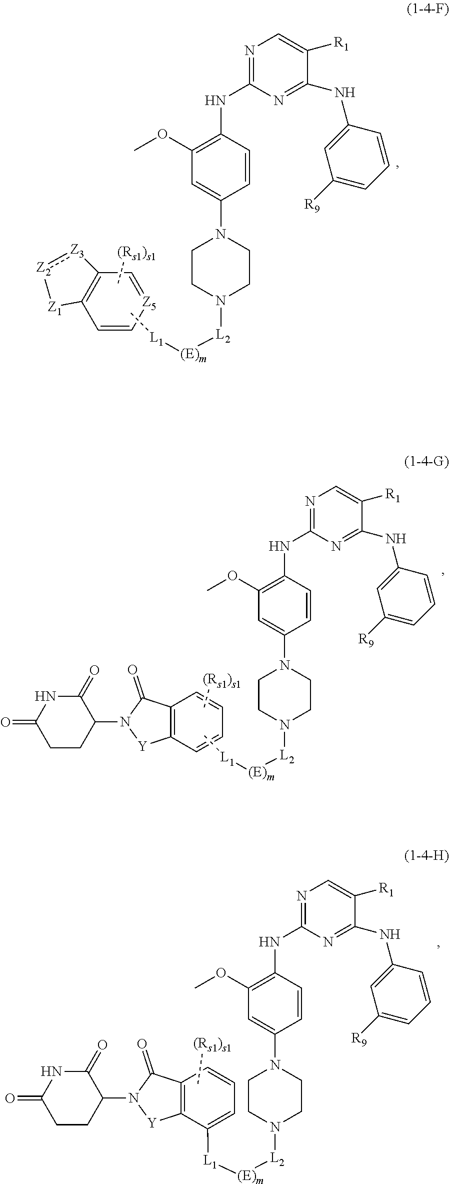 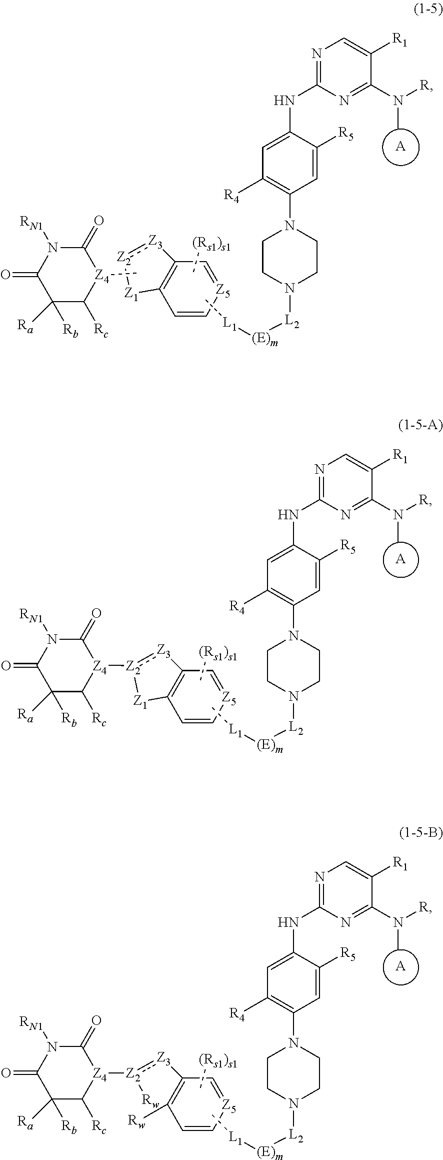 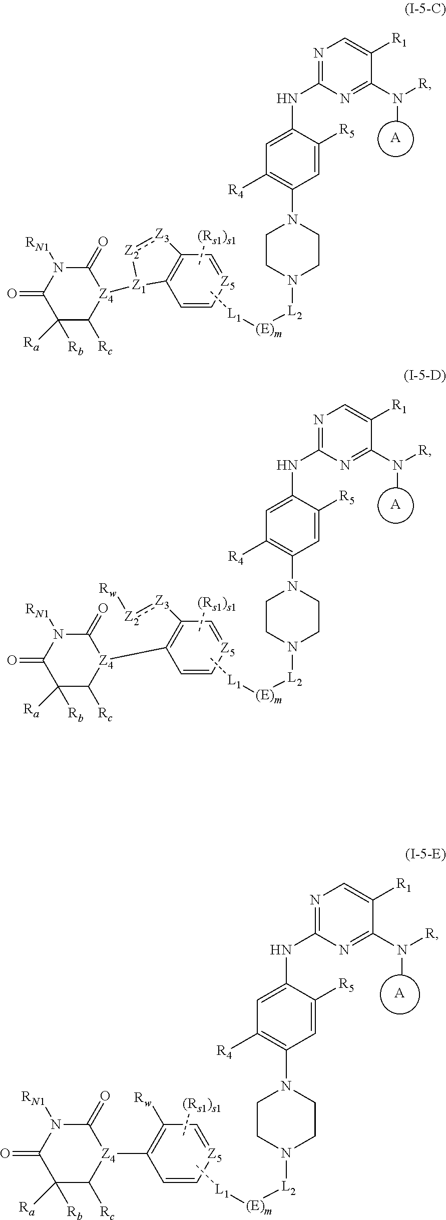 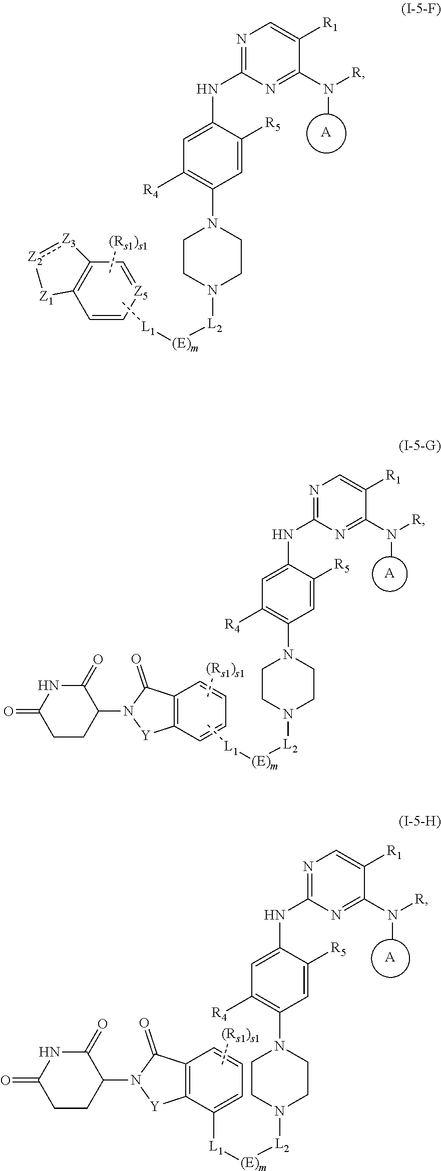 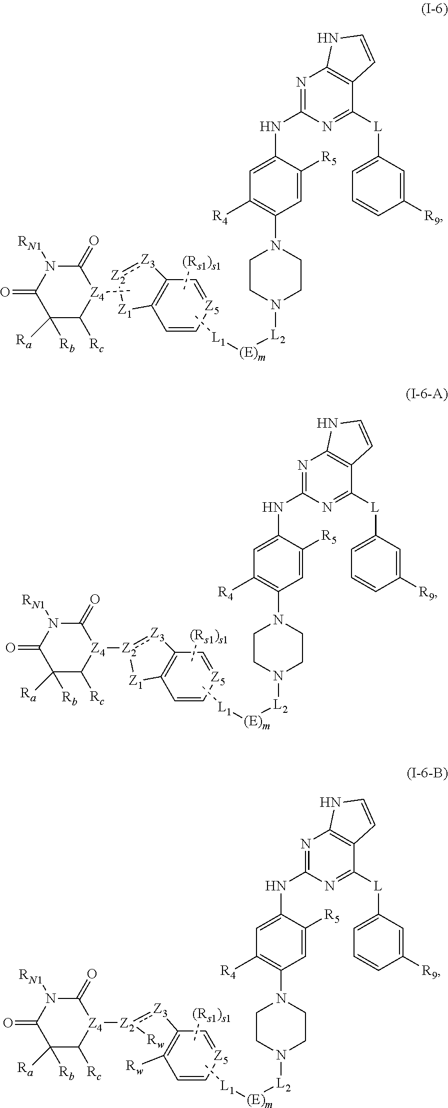 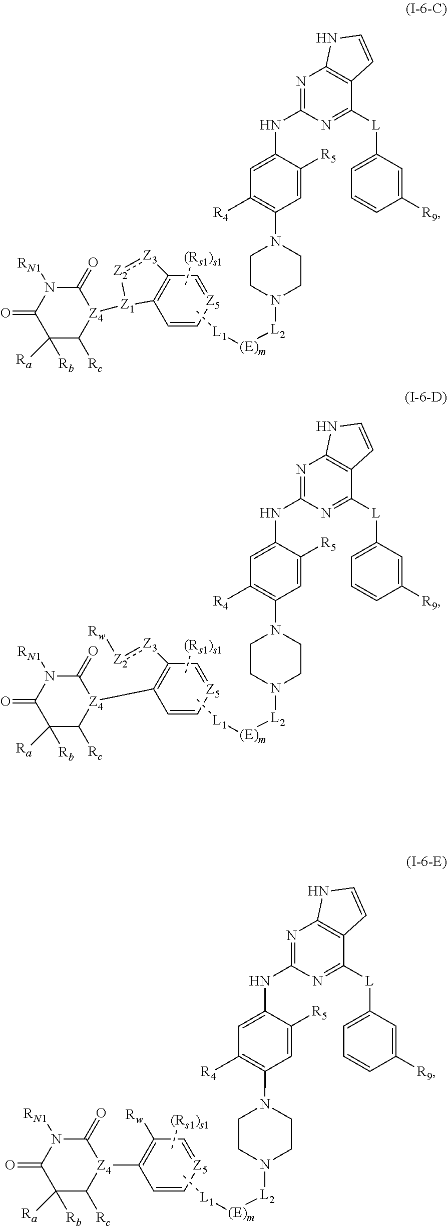 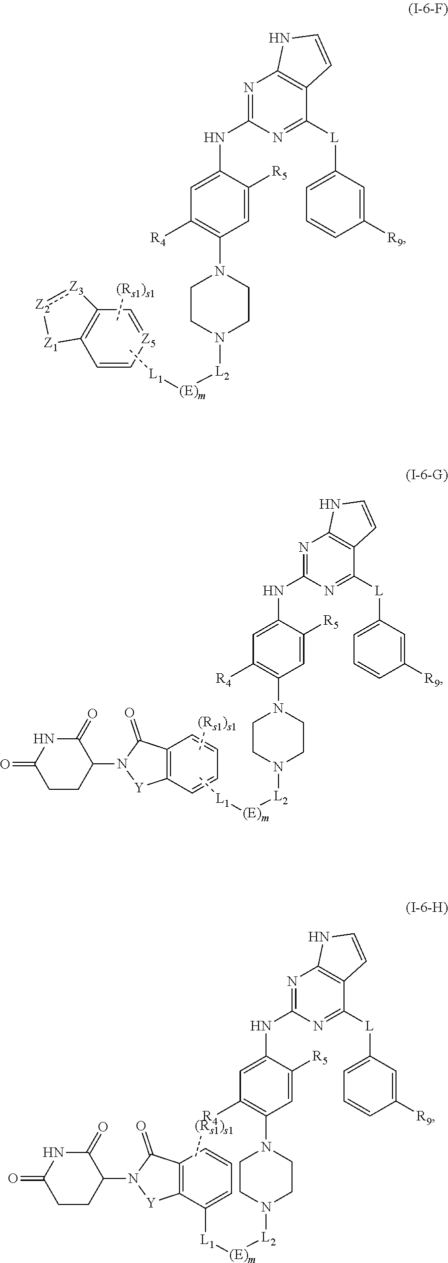
wherein the definition of Y is the same as that of Z₁, and other groups are as defined in claim 2.

9. The compound of general formula (I), or the pharmaceutically acceptable salt, enantiomer, diastereomer, racemate, solvate, hydrate, polymorph, prodrug, or isotopic variant thereof, or mixture thereof according to claim 8, wherein the compound of general formula (I) is a compound of general formula (I-1), (I-1-A), (I-1-B), (I-1-C), (I-1-D), (I-1-E), (I-1-F), (I-1-G), (I-1-H) or (I-1-I),
wherein
R₁ is selected from —Cl, —Br, —CF₃ or —CH═CH₂; alternatively, R₁ is selected from —Cl or —Br;
R₄ is H or -Me;
R₈ is H, —NHMe, —NHC(O)CH₂CH₃ or —NH-cyclopropyl;
R₉ is H, —F, —Cl, —Br, -Me, —CF₃, —OMe, —OCF₃, —CN, —NHC(O)CH₂CH₃, —NHS(O)₂CH₂CH₃ or —NHC(O)CH═CH₂;
X is —C(Rₓ)═ or —N═;
Rₓ is H, or Rₓ and Rₛ are taken together with the C atoms to which they are attached to form pyrazinyl;
and other groups are as defined in claim 8,
or the compound of general formula (I) is a compound of general formula (I-G): 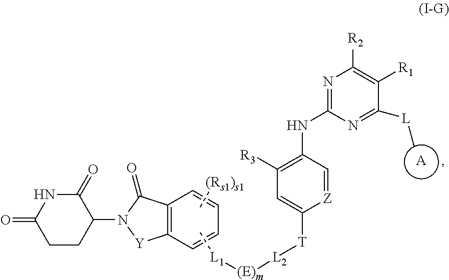
wherein
ring A is the following group: 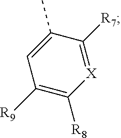
wherein
R₇ is —P(O)(C₁₋₆ alkyl)₂;
R⁸ is H;
R₉ is selected from H, halogen, C₁₋₆ alkyl, —CN or C₁₋₆ haloalkyl;
X is —C(Rₓ)═;
wherein Rₓ is H, or Rₓ and R₈ are taken together with the C atoms to which they are attached to form 5- to 6-membered heteroaryl; alternatively form pyrazinyl;
Y is C atom, which is optionally substituted with one or two R_{Z1}, wherein R_{Z1} is H, CN or halogen: or two R_{Z1} are taken together with Y to form C═O; alternatively, Y is CH₂ or C═O; alternatively, Y is CH₂;
L₁ is —CₐR^{#}R^{#′—C}_{b}R^{#}R^{#}′—;
L₂ is selected from bond, —CR^{#}R^{#}′— or —CₐR^{#}R^{#}′—C_{b}R^{#}R^{#}′—,
wherein one of CₐR^{#}R^{#}′ or C_{b}R^{#}R^{#}′ can be replaced by O, S(O)ₚ or NR^{#};
E is independently selected from: bond or —CₑR^{#}R^{#}′—C_{d}R^{#}R^{#}′—CₑR^{#}R^{#}′;
wherein one of C_{c}R^{#}R^{#}′, C_{d}R^{#}R^{#}′ or CₑR^{#}R^{#}′, or both of C_{c}R^{#}R′ and CₑR^{#}R^{#}′ can be replaced by O, S(O)ₚ or NR^{#};
p is 0, 1 or 2;
R^{#} is H, halogen, C₁₋₆ alkyl, C₁₋₆ haloalkyl, C₂₋₆ alkenyl or C₂₋₆ alkynyl;
R^{#}′ is H, halogen, C₁₋₆ alkyl, C₁₋₆ haloalkyl, C₂₋₆ alkenyl or C₂₋₆ alkynyl;
or, R^{#} and R^{#} on adjacent atoms can be taken together to form bond, and R^{#}′ and R^{#}′ on adjacent atoms can be taken together to form bond;
or, R^{#} and R^{#}′ on the same or different atoms can be taken together to form ═O;
m is 0, 1, 2, 3, 4 or 5;
Rₛ₁ is selected from H, CN, halogen, OH, NH₂, C₂₋₆ alkenyl, C₂₋₆ alkynyl, —O—C₁₋₆ alkyl, —O—C₁₋₆ haloalkyl, —NH—C₁₋₆ alkyl, C₁₋₆ alkyl or C₁₋₆ haloalkyl;
s1 is 0, 1, 2 or 3;
L is —NR—; wherein R is H or C₁₋₆ alkyl;
Z is —C(R₄)═;
T is or 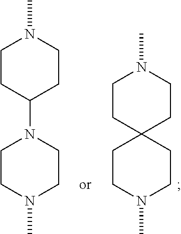
R₁ is selected from H, halogen, cyano, C₁₋₆ alkyl, C₁₋₆ haloalkyl, C₂₋₆ alkenyl or C₂₋₆ alkynyl;
R₂ is H, halogen, hydroxyl, amino, C₁₋₆ alkyl or C₁₋₆ haloalkyl;
R₃ is selected from H, —O—C₁₋₆ alkyl or —O—C₁₋₆ haloalkyl;
R₄ is selected from H, halogen, C₁₋₆ alkyl or C₁₋₆ haloalkyl;
if the above groups are H or H-containing groups, the one or more H atom(s) may be substituted with D atom(s),
or the compound of general formula (I) is a compound of general formula (I-1-G), (I-1-H), (I-1-H′), or (I-1-H″): 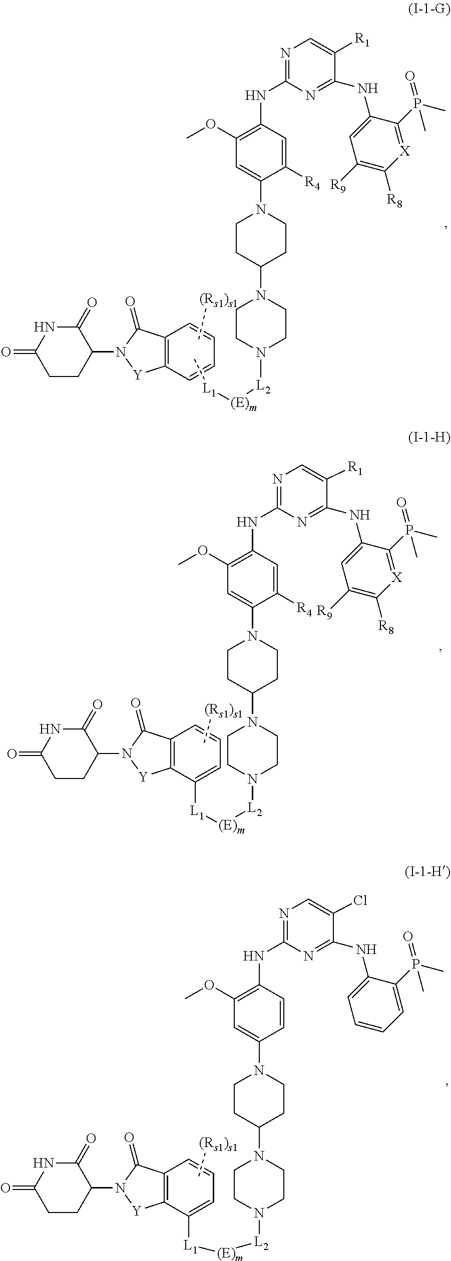 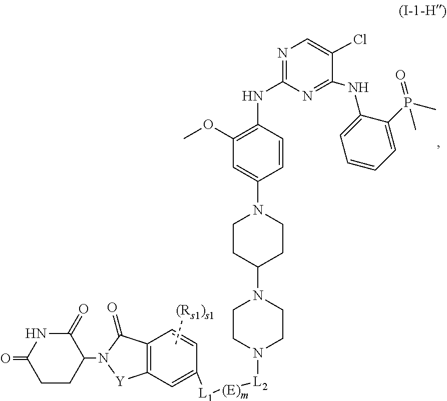
wherein
X is —CH═;
Y is C atom, which is optionally substituted with one or two R_{Z1}, wherein R_{Z1} is H, CN or halogen; or two R_{Z1} are taken together with Y to form C═O; alternatively, Y is CH₂ or C═O;
R₁ is selected from H, halogen, cyano, C₁₋₆ alkyl, C₁₋₆ haloalkyl, C₂₋₆ alkenyl or C₂₋₆ alkynyl;
alternatively, R₁ is H or halogen;
R₄ is selected from H, halogen, C₁₋₆ alkyl or C₁₋₆ haloalkyl; alternatively, R₄ is H;
R⁸ is H;
R₉ is selected from H, halogen, C₁₋₆ alkyl, —CN or C₁₋₆ haloalkyl; alternatively, R₉ is H;
L₁ is selected from —CH₂CH₂—, —CH═CH—, —C≡C—, —OCH₂—, —SCH₂—, —S(O)CH₂—, —S(O)₂CH₂—, —NHCH₂—, —N(Me)CH₂—, —C(O)CH₂—, —CH₂C(O)—, —OC(O)—, —SC(O)—, —NHC(O)— or —N(Me)C(O)—, and one or more H atom(s) in the above groups can be substituted with D atom(s); alternatively, L₁ is selected from —CH₂CH₂—, —CH═CH—, —C≡C—, —OCH₂— or —NHCH₂—; alternatively, L₁ is selected from —CH₂CH₂—, —C≡C— or —OCH₂—;
L₂ is selected from bond, —CH₂— or —CH₂CH₂—, and one or more H atom(s) in the above groups can be substituted with D atom(s);
E is —CH₂CH₂CH₂—, —CH₂CH₂O—, —CH₂OCH₂—, —OCH₂CH₂—, —CH₂CH₂S—, —CH₂SCH₂— or —SCH₂CH₂—, and one or more H atom(s) in the above groups can be substituted with D atom(s);
alternatively, E is —CH₂CH₂CH₂—;
m is 0, 1, 2 or 3; alternatively, the chain length of -L₁-(E)ₘ-L₂- is alternatively 4 to 12 bond lengths, still alternatively 5, 6, 7, 8, 9 or 10 bond lengths;
Rₛ₁ is selected from H, CN, halogen, OH, NH₂, C₂₋₆ alkenyl, C₂₋₆ alkynyl, —O—C₁₋₆ alkyl, —O—C₁₋₆ haloalkyl, —NH—C₁₋₆ alkyl, C₁₋₆ alkyl or C₁₋₆ haloalkyl; alternatively, Rₛ₁ is H, CN or halogen;
s1 is 0, 1 or 2,
or the compound of general formula (I) is a compound of general formula (I-1-G), (I-1-H), (I-1-H′), or (I-1-H″): 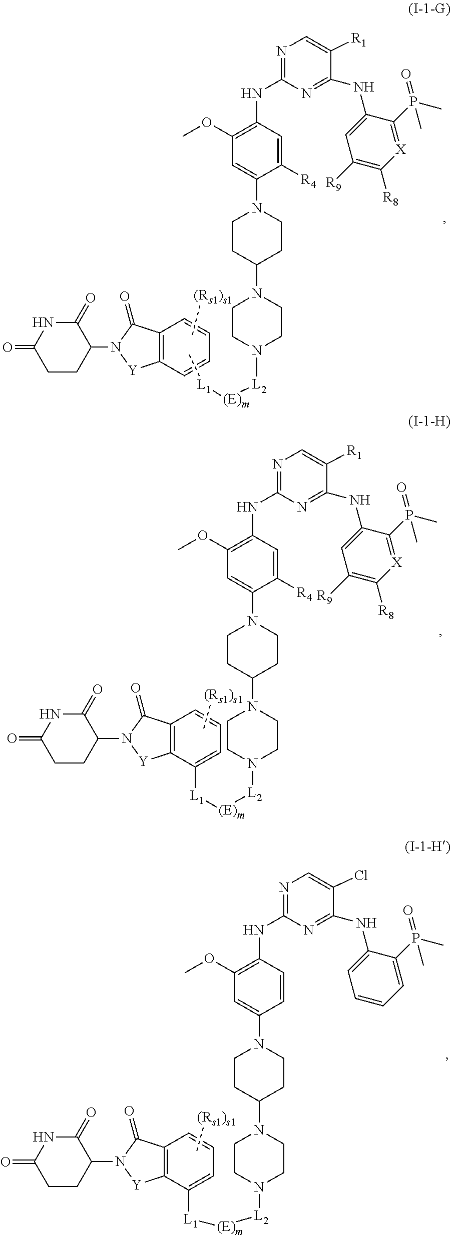 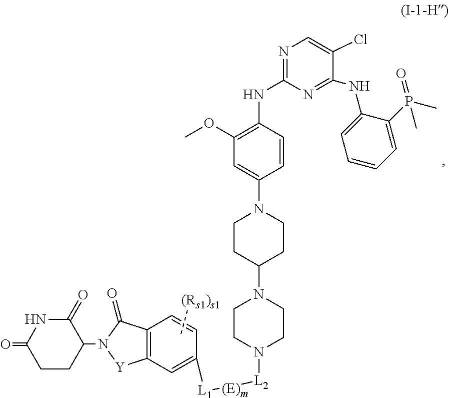
X is —CH═;
Y is C atom, which is optionally substituted with one or two R_{Z1}, wherein R_{Z1} is H, CN or halogen; or two R_{Z1} are taken together with Y to form C═O; alternatively, Y is CH₂;
R₁ is selected from H, halogen, cyano, C₁₋₆ alkyl, C₁₋₆ haloalkyl, C₂₋₆ alkenyl or C₂₋₆ alkynyl;
alternatively, R₁ is halogen;
R₄ is selected from H, halogen, C₁₋₆ alkyl or C₁₋₆ haloalkyl; alternatively, R₄ is H;
R⁸ is H;
R₉ is selected from H, halogen, C₁₋₆ alkyl, —CN or C₁₋₆ haloalkyl; alternatively, R₉ is H;
L₁ is selected from —CH₂CH₂—, —CH═CH—, —C≡C—, —OCH₂—, —SCH₂—, —S(O)CH₂—, —S(O)₂CH₂—, —NHCH₂—, —N(Me)CH₂—, —C(O)CH₂—, —CH₂C(O)—, —OC(O)—, —SC(O)—, —NHC(O)— or —N(Me)C(O)—, and one or more H atom(s) in the above groups can be substituted with D atom(s); alternatively, L₁ is selected from —CH₂CH₂—, —CH═CH—, —C≡C—, —OCH₂— or —NHCH₂—; alternatively, L₁ is selected from —CH₂CH₂—, —C≡C—, —OCH₂— or —NHCH₂—;
L₂ is selected from bond, —CH₂— or —CH₂CH₂—, and one or more H atom(s) in the above groups can be substituted with D atom(s);
E is —CH₂CH₂CH₂—, —CH₂CH₂O—, —CH₂OCH₂—, —OCH₂CH₂—, —CH₂CH₂S—, —CH₂SCH₂— or —SCH₂CH₂—, and one or more H atom(s) in the above groups can be substituted with D atom(s);
alternatively, E is —CH₂CH₂CH₂—;
m is 0, 1, 2 or 3; alternatively, the chain length of -L₁-(E)ₘ-L₂- is alternatively 4 to 14 bond lengths, still alternatively 5, 6, 7, 8, 9 or 10 bond lengths;
Rₛ₁ is selected from H, CN, halogen, OH, NH₂, C₂₋₆ alkenyl, C₂₋₆ alkynyl, —O—C₁₋₆ alkyl, —O—C₁₋₆ haloalkyl, —NH—C₁₋₆ alkyl, C₁₋₆ alkyl or C₁₋₆ haloalkyl; alternatively, Rₛ₁ is H or halogen;
s1 is 0, 1 or 2,
or the compound of general formula (I) is a compound of general formula (I-1-G), (I-1-H), (I-1-H′), or (I-1-H″: 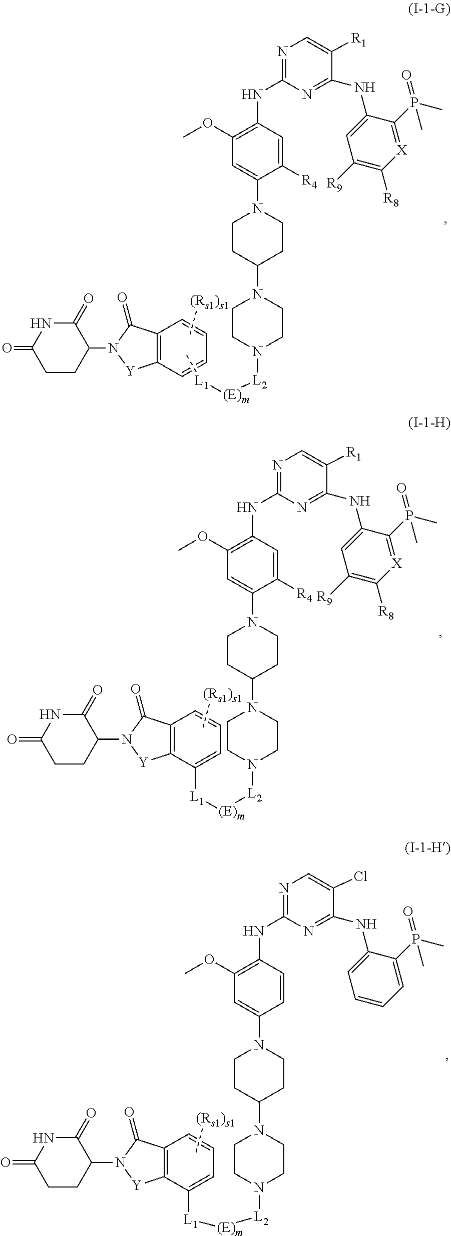 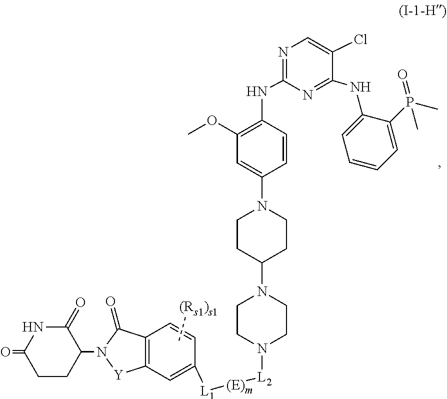
wherein
X is —CH═;
Y is C atom, which is optionally substituted with one or two R_{Z1}, wherein R_{Z1} is H, CN or halogen; or two R_{Z1} are taken together with Y to form C═O; alternatively, Y is CH₂ or C≡O;
R₁ is selected from H, halogen, cyano, C₁₋₆ alkyl, C₁₋₆ haloalkyl, C₂₋₆ alkenyl or C₂₋₆ alkynyl;
alternatively, R₁ is halogen;
R₄ is selected from H, halogen, C₁₋₆ alkyl or C₁₋₆ haloalkyl; alternatively, R₄ is H;
R₉ is selected from H, halogen, C₁₋₆ alkyl, —CN or C₁₋₆ haloalkyl; alternatively, R₉ is H,
L₁ is selected from —CH₂CH₂—, —CH═CH—, —C≡C—, —OCH₂—, —SCH₂—, —S(O)CH₂—, —S(O)₂CH₂—, —NHCH₂—, —N(Me)CH₂—, —C(O)CH₂—, —CH₂C(O)—, —OC(O)—, —SC(O)—, —NHIC(O)— or —N(Me)C(O)—, and one or more H atom(s) in the above groups can be substituted with D atom(s), alternatively, L₁ is selected from —CH₂CH₂—, —CH═CH—, —C≡C—, —OCH₂— or —NHCH₂—; alternatively, L₁ is selected from —C≡C—, —OCH₂— or —NHCH₂—;
L₂ is selected from bond, —CH₂— or —CH₂CH₂—, and one or more H atom(s) in the above groups can be substituted with D atom(s);
E is —CH₂CH₂CH₂—, —CH₂CH₂O—, —CH₂OCH₂—, —OCH₂CH₂—, —CH₂CH₂S—, —CH₂SCH₂— or —SCH₂CH₂—, and one or more H atom(s) in the above groups can be substituted with D atom(s);
alternatively, E is —CH₂CH₂CH₂—;
m is 1, 2 or 3; alternatively, the chain length of -L₁-(E)ₘ-L₂- is less than 14 bond lengths;
alternatively, the chain length is 5-10 bond lengths;
Rₛ₁ is selected from H, CN, halogen, OH, NH₂, C₂₋₆ alkenyl, C₂₋₆ alkynyl, —O—C₁₋₆ alkyl, —O—C₁₋₆ haloalkyl, —NH—C₁₋₆ alkyl, C₁₋₆ alkyl or C₁₋₆ haloalkyl; alternatively, Rₛ₁ is H or halogen;
s1 is 0, 1 or 2,
or the compound of general formula (I) is a compound of general formula (I-1-I), (I-1-I′), (I-1-I″) or (I-1-I″′), 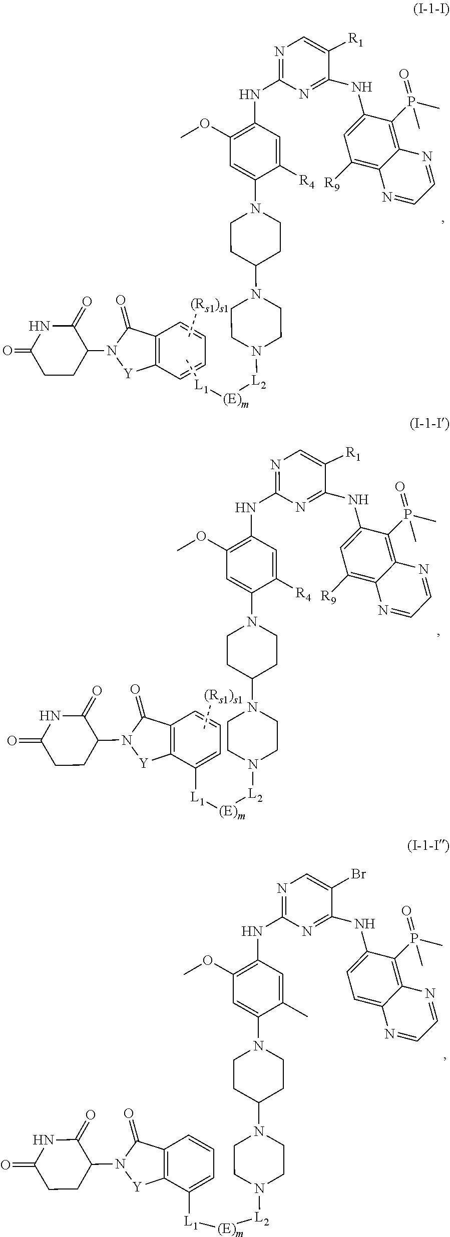 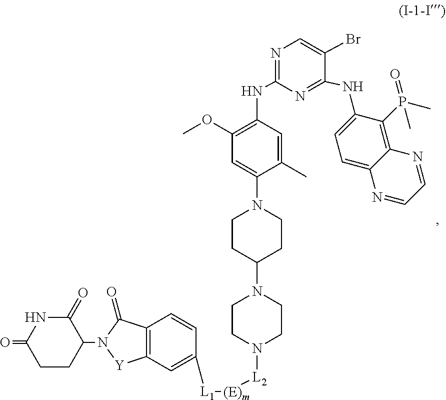
wherein
Y is C atom, which is optionally substituted with one or two R_{Z1}, wherein R_{Z1} is H, CN or halogen; or two R_{Z1} are taken together with Y to form C═O; alternatively, Y is CH₂ or C≡O;
R₁ is selected from H, halogen, cyano, C₁₋₆ alkyl, C₁₋₆ haloalkyl, C₂₋₆ alkenyl or C₂₋₆ alkynyl; alternatively, R₁ is halogen;
R₄ is selected from H, halogen, C₁₋₆ alkyl or C₁₋₆ haloalkyl; alternatively, R₄ is selected from C₁, 6 alkyl or C₁₋₆ haloalkyl;
R₉ is selected from H, halogen, C₁₋₆ alkyl, —CN or C₁₋₆ haloalkyl; alternatively, R₉ is H;
L₁ is selected from —CH₂CH₂—, —CH═CH—, —C≡C—, —OCH₂—, —SCH₂—, —S(O)CH₂—, —S(O)₂CH₂—, —NHCH₂—, —N(Me)CH₂—, —C(O)CH₂—, —CH₂C(O)—, —OC(O)—, —SC(O)—, —NHC(O)— or —N(Me)C(O)—; alternatively, L₁ is selected from —CH₂CH₂—, —CH═CH—, —C≡C— or —OCH₂—; alternatively, L₁ is selected from —CH₂CH₂—, —C≡C— or —OCH₂—,
L₂ is selected from bond, —CH₂— or —CH₂CH₂—;
E is —CH₂CH₂CH₂—, —CH₂CH₂O—, —CH₂OCH₂—, —OCH₂CH₂—, —CH₂CH₂S—, —CH₂SCH₂— or —SCH₂CH₂—; alternatively, E is —CH₂CH₂CH₂—,
m is 1 or 2; alternatively, the chain length of -L₁-(E)ₘ-L₂- is 4 to 14 bond lengths, still alternatively 5, 6, 7, 8, 9 or 10 bond lengths,
Rₛ₁ is selected from H, CN, halogen, OH, NH₂, C₂₋₆ alkenyl, C₂₋₆ alkynyl, —O—C₁₋₆ alkyl, —O—C₁₋₆ haloalkyl, —NH—C₁₋₆ alkyl, C₁₋₆ alkyl or C₁₋₆ haloalkyl; alternatively, Rₛ₁ is H, halogen or CN;
s1 is 0, 1 or 2,
or the compound of general formula (I) is a compound of general formula (I-1-I), (I-1-I′), (I-1-I″, or (I-1-I″′), 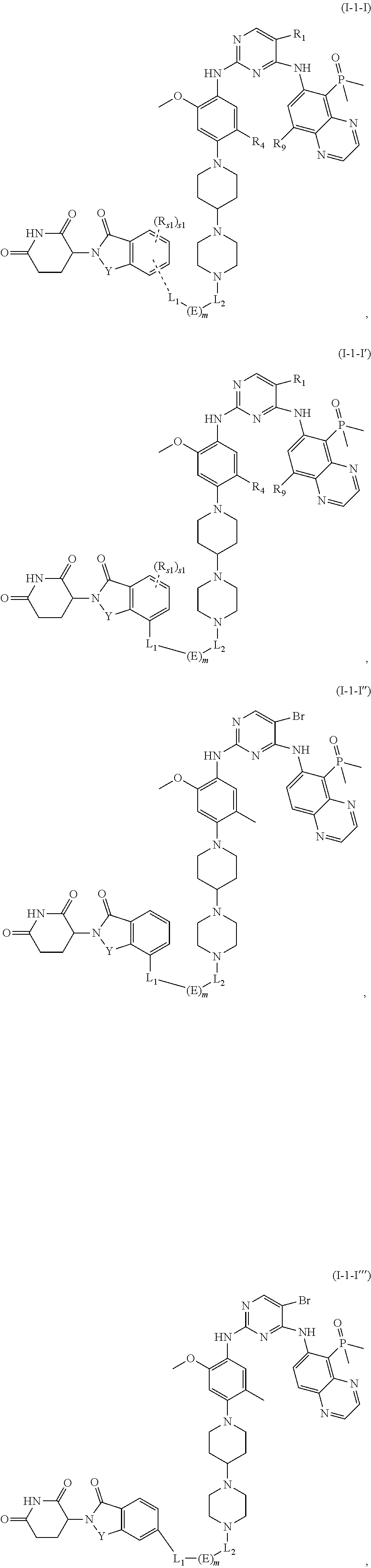
wherein
Y is C atom, which is optionally substituted with one or two R_{Z1}, wherein R_{Z1} is H, CN or halogen; or two R_{Z1} are taken together with Y to form C═O; alternatively, Y is CH₂;
R₁ is selected from H, halogen, cyano, C₁₋₆ alkyl, C₁₋₆ haloalkyl, C₂₋₆ alkenyl or C₂₋₆ alkynyl;
alternatively, R₁ is halogen;
R₄ is selected from H, halogen, C₁₋₆ alkyl or C₁₋₆ haloalkyl; alternatively, R₄ is selected from C₁, 6 alkyl or C₁₋₆ haloalkyl;
R₉ is selected from H, halogen, C₁₋₆ alkyl, —CN or C₁₋₆ haloalkyl; alternatively, R₉ is H;
L₁ is selected from —CH₂CH₂—, —CH═CH—, —C≡C—, —OCH₂—, —SCH₂—, —S(O)CH₂—, —S(O)₂CH₂—, —NHCH₂—, —N(Me)CH₂—, —C(O)CH₂—, —CH₂C(O)—, —OC(O)—, —SC(O)—, —NHC(O)— or —N(Me)C(O)—; alternatively, L₁ is selected from —CH₂CH₂—, —CH═CH—, —C≡C— or —OCH₂—; alternatively, L₁ is selected from —C≡C— or —OCH₂—;
L₂ is selected from bond, —CH₂— or —CH₂CH₂—;
E is —CH₂CH₂CH₂—;
m is 1 or 2; alternatively, the chain length of -L₁-(E)ₘ-L₂- is less than 10 bond lengths;
alternatively, the chain length is 6 to 9 bond lengths, still alternatively 6, 7, 8 or 9 bond lengths;
Rₛ₁ is selected from H, CN, halogen, OH, NH₂, C₂₋₆ alkenyl, C₂₋₆ alkynyl, —O—C₁₋₆ alkyl, —O—C₁₋₆ haloalkyl, —NH—C₁₋₆ alkyl, C₁₋₆ alkyl or C₁₋₆ haloalkyl; alternatively, Rₛ₁ is H or halogen;
s1 is 0, 1 or 2,
or the compound of general formula (I) is a compound of general formula (I-2-I), (I-2-I′), (I-2-I″), or (I-2-I″′): 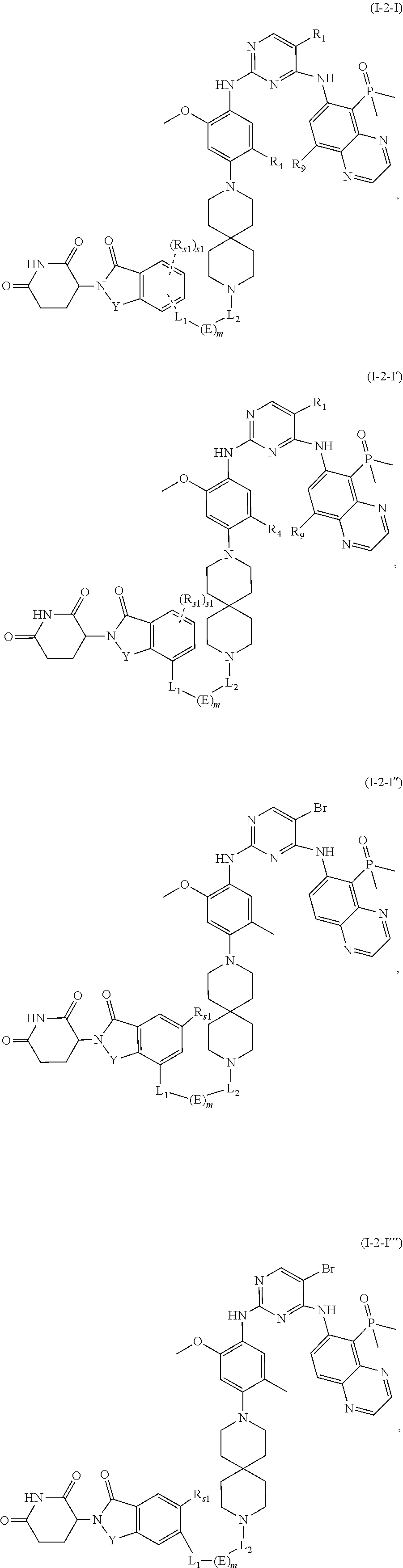
wherein
Y is C atom, which is optionally substituted with one or two R_{Z1}, wherein R_{Z1} is H, CN or halogen; or two R_{Z1} are taken together with Y to form C═O; alternatively, Y is CH₂;
R₁ is selected from H, halogen, cyano, C₁₋₆ alkyl, C₁₋₆ haloalkyl, C₂₋₆ alkenyl or C₂₋₆ alkynyl;
alternatively, R₁ is halogen;
R₄ is selected from H, halogen, C₁₋₆ alkyl or C₁₋₆ haloalkyl; alternatively, R₄ is selected from C₁, 6 alkyl or C₁₋₆ haloalkyl;
R₉ is selected from H, halogen, C₁₋₆ alkyl, —CN or C₁₋₆ haloalkyl; alternatively, R₉ is H;
L₁ is selected from —CH₂CH₂—, —CH═CH—, —C≡C—, —OCH₂—, —SCH₂—, —S(O)CH₂—, —S(O)₂CH₂—, —NHCH₂—, —N(Me)CH₂—, —C(O)CH₂—, —CH₂C(O)—, —OC(O)—, —SC(O)—, —NHC(O)— or —N(Me)C(O)—; alternatively, L₁ is selected from —CH₂CH₂—, —CH═CH—, —C≡C— or —OCH₂—; alternatively, L₁ is selected from —C≡C— or —OCH₂—;
L₂ is selected from bond, —CH₂— or —CH₂CH₂—,
E is —CH₂CH₂CH₂—, —CH₂CH₂O—, —CH₂OCH₂—, —OCH₂CH₂—, —CH₂CH₂S—, —CH₂SCH₂— or —SCH₂CH₂—; alternatively, E is —CH₂CH₂CH₂—,
m is 1 or 2; alternatively, the chain length of -L₁-(E)ₘ-L₂- is less than 10 bond lengths: alternatively, the chain length is 6 to 9 bond lengths, still alternatively 6, 7, 8 or 9 bond lengths;
Rₛ₁ is selected from H, CN, halogen, OH, NH₂, C₂₋₆ alkenyl, C₂₋₆ alkynyl, —O—C₁₋₆ alkyl, —O—C₁₋₆ haloalkyl, —NH—C₁₋₆ alkyl, C₁₋₆ alkyl or C₁₋₆ haloalkyl; alternatively, Rₛ₁ is H or halogen;
s1 is 0, 1 or 2,
or the compound of general formula (I) is a compound of general formula (I-2), (I-2-A), (I-2-B), (I-2-C), (I-2-D), (I-2-E), (I-2-F), (I-2-G), (I-2-H) or (I-2-I);
wherein
R₁ is —Cl, —Br or —CH═CH₂;
R₄ is H or -Me;
R₈ is H, —NHMe, —NHC(O)CH₂CH₃ or —NH-cyclopropyl;
R₉ is H, —F, —Cl, —Br, -Me, —CF₃, —OMe, —OCF₃, —CN, —NHC(O)CH₂CH₃, —NHS(O)₂CH₂CH₃ or —NHC(O)CH═CH₂;
X is —C(Rₓ)═;
Rₓ is H, or Rₓ and R₈ are taken together with the C atoms to which they are attached to form pyrazinyl;
and other groups are as defined in claim 8,
or the compound of general formula (I) is a compound of general formula (I-3), (I-3-A), (I-3-B), (I-3-C), (I-3-D), (I-3-E), (I-3-F), (I-3-G) or (I-3-H);
wherein
R₁ is H, —Cl or —CH═CH₂;
R₄ is —NHC(O)CH═CH₂;
R₅ is -Me;
R₆ is -Me;
or R₅ and R₆ are connected to form —CH₂CH₂—;
R₇ is -Me or cyclopropyl;
and other groups are as defined in claim 8,
or the compound of general formula (I) is a compound of general formula (I-4), (I-4-A), (I-4-B), (I-4-C), (I-4-D), (I-4-E), (I-4-F), (I-4-G) or (I-4-H);
wherein
R₁ is —CF₃;
R₉ is H, —F, —Cl, —Br, -Me, —CF₃, —OMe, —OCF₃, —CN, —NHC(O)CH═CH₂ or —NH-cyclopropyl;
and other groups are as defined in claim 8,
or the compound of general formula (I) is a compound of general formula (I-6), (I-6-A), (I-6-B), (I-6-C), (I-6-D), (I-6-E), (I-6-F), (I-6-G) or (I-6-H);
wherein
L is —O— or —NH—;
R₃ is H or —OMe;
R₄ is H or —F;
R₉ is —CF₃, —OMe, —OCF₃, —CN, —NHC(O)CH₂CH₃ or —NHC(O)CH═CH₂;
and other groups are as defined in claim 8.

10.-19. (canceled)

20. The compound of general formula (I), or the pharmaceutically acceptable salt, enantiomer, diastereomer, racemate, solvate, hydrate, polymorph, prodrug, or isotopic variant thereof, or mixture thereof according to claim 8, wherein the compound of general formula (I) is a compound of general formula (I-5), (I-5-A), (I-5-B), (I-5-C), (I-5-D), (I-5-E), (I-5-F), (I-5-G) or (I-5-H):
wherein
ring A is the following optionally substituted groups: C₃₋₇ cycloalkyl or C₆₋₁₀ aryl, wherein the substituent is selected from —F, —Cl, —Br, -Me, —OMe, —CF₃, —OCF₃, —CN, —NHMe, —P(O)Me₂, —NHC(O)CH₂CH₃, —C(O)CH═CH₂, —NHS(O)₂CH₂CH₃, —NH-cyclopropyl, —NHC(O)CH═CH₂ or —C(O)CH₂CH₃; ring A is alternatively the following groups: 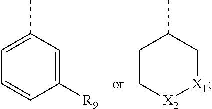
wherein
R₉ is H, —F, —Cl, —Br, -Me, —CF₃, —OMe, —OCF₃, —CN, —NHC(O)CH₂CH₃, —NHS(O)₂CH₂CH₃ or —NHC(O)CH═CH₂;
X₁ is —CH₂— or —N(R_{X1})—;
X₂ is —CH(R_{X2})— or —N(R_{X2})—;
wherein
R_{X1} is —C(O)CH₂CH₃ or —C(O)CH═CH₂;
R_{X2} is H, -Me, —OMe, —NHMe, —NHS(O)₂CH₂CH₃, —C(O)CH₂CH₃ or —NHC(O)CH₂CH₃; [symbol]
represents the point of attachment;
R₁ and R are taken together with the atoms to which they are attached to form optionally substituted 5- to 6-membered heterocyclyl, wherein the substituent is selected from halogen, oxo, -iPr, -Et, or phenyl, wherein the phenyl is mono- or poly-substituted with halogen; R₁ and R are alternatively taken together to form: —C(O)N(R_{N})C(O)— or —C(CH₃)═C(R_{N})C(O)—;
wherein
R_{N} is selected from -iPr, -Et or 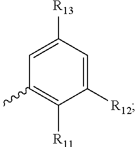
R₁₁ is —Cl, —Br;
R₁₂ is H or —F;
R₁₃ is H or —F; [symbol]
represents the point of attachment;
R₃ is H or —OMe;
R₄ is H or -Me;
and other groups are as defined in claim 8.

21. The compound of general formula (I), or the pharmaceutically acceptable salt, enantiomer, diastereomer, racemate, solvate, hydrate, polymorph, prodrug, or isotopic variant thereof, or mixture thereof according to claim 20, wherein the compound of general formula (I) is a compound of general formula (I-5-1) or (I′-5-1): 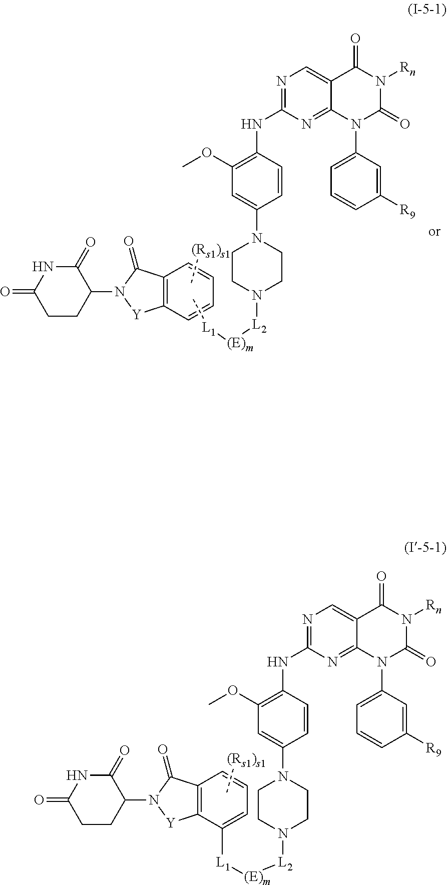
wherein
R₉ is —NHC(O)CH₂CH₃ or —NHC(O)CH═CH₂;
R_{N} is -iPr or -Et;
and other groups are as defined in claim 20.

22. The compound of general formula (I), or the pharmaceutically acceptable salt, enantiomer, diastereomer, racemate, solvate, hydrate, polymorph, prodrug, or isotopic variant thereof, or mixture thereof according to claim 20, wherein the compound of general formula (I) is a compound of general formula (I-5-2) or (I′-5-2): 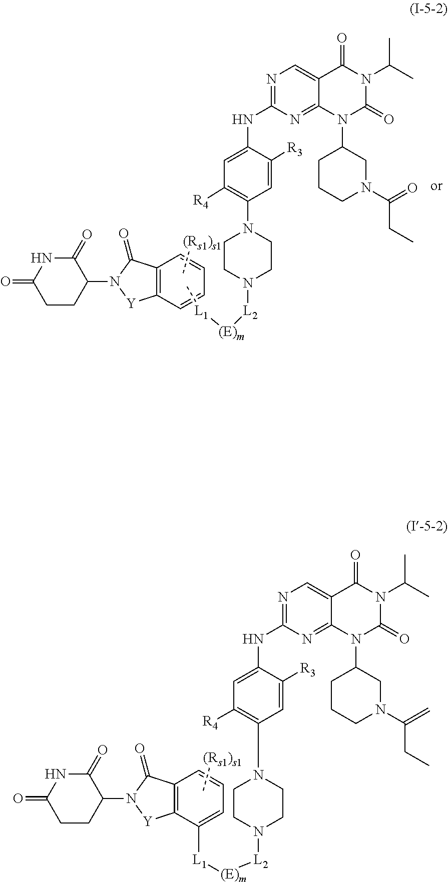
wherein
R₃ is H or —OMe;
R₄ is H or -Me;
and other groups are as defined in claim 20.

23. The compound of general formula (I), or the pharmaceutically acceptable salt, enantiomer, diastereomer, racemate, solvate, hydrate, polymorph, prodrug, or isotopic variant thereof, or mixture thereof according to claim 20, wherein the compound of general formula (I) is a compound of general formula (I-5-3) or (I′-5-3): 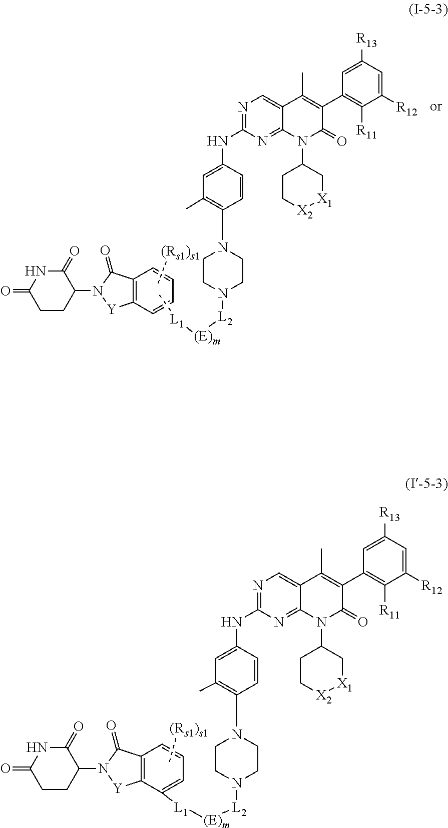
wherein
X₁ is —CH₂— or —N(R_{X1})—;
X₂ is —CH(R_{X2})— or —N(R_{X2})—;
wherein
R_{X1} is —C(O)CH₂CH₃;
R_{X2} is H, -Me, —OMe, —NHMe, —C(O)CH₂CH₃, —NHS(O)₂CH₂CH₃ or —NHC(O)CH₂CH₃;
R₁₁ is —Cl or —Br;
R₁₂ is H or —F;
R₁₃ is H or —F;
and other groups are as defined in claim 20.

24. (canceled)

25. The compound of general formula (X), or the pharmaceutically acceptable salt, enantiomer, diastereomer, racemate, solvate, hydrate, polymorph, prodrug, or isotopic variant thereof, or mixture thereof according to claim 1,
wherein 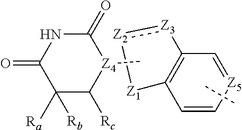
is selected from the following groups: 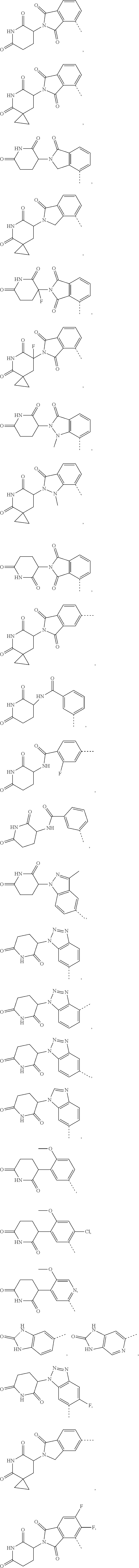 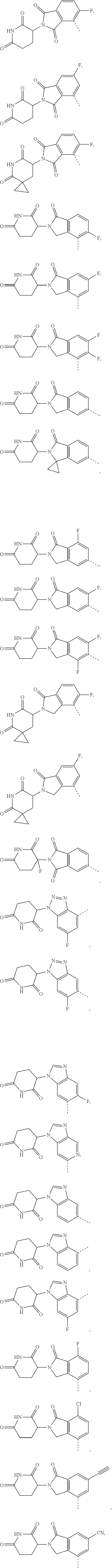 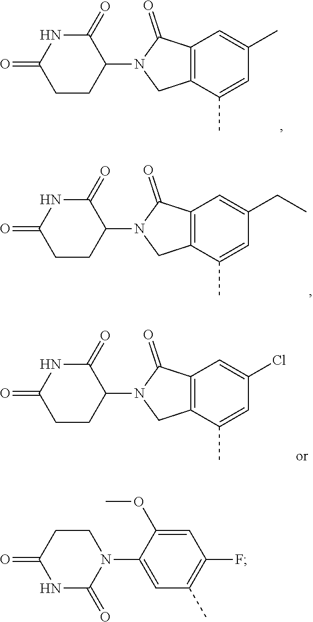
and one or more H atom(s) in the above groups can be substituted with D atom(s).

26. The compound of general formula (X), or the pharmaceutically acceptable salt, enantiomer, diastereomer, racemate, solvate, hydrate, polymorph, prodrug, or isotopic variant thereof, or mixture thereof according to claim 1, wherein L₁ and L₂ are independently selected from bond, —O—, —S—, —S(O)—, —S(O)₂—, —S(O)(═NH)—, —S(O)(═NMe)-, 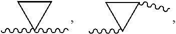
—NH—, —N(Me)-, 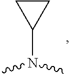
—N(CF₃)—, —CH₂—, —CH(OMe)-, —CH(Cl)—, —CH(F)—, —CF₂, —CH(CF₃)—, —C(O)—, —CH₂CH₂—, —CH═CH—, —C≡C—, —OCH₂—, —CH₂O—, —SCH₂—, —CH₂S—, —S(O)CH₂—, —CH₂S(O)—, —S(O)₂CH₂—, —CH₂S(O)₂—, —NHCH₂—, —N(Me)CH₂—, —CH₂NH—, —CH₂N(Me)-, —C(O)CH₂—, —CH₂C(O)—, —C(O)CMe₂-, —CMe₂C(O)—, —OC(O)—, —C(O)O—, —SC(O)—, —C(O)S—, —NHC(O)—, —N(Me)C(O)—, —C(O)NH—, —C(O)N(Me)-, —S(O)═NH—, —NH═S(O)—, —N═S(O)Me-, —S(O)Me=N—, or 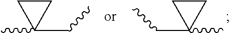
and one or more H atom(s) in the above groups can be substituted with D atom(s).

27. The compound of general formula (I), or the pharmaceutically acceptable salt, enantiomer, diastereomer, racemate, solvate, hydrate, polymorph, prodrug, or isotopic variant thereof, or mixture thereof according to claim 1, wherein E is selected from bond, —CH₂CH₂CH₂—, —CH₂CH═CH—, —CH═CHCH₂—, —CH₂C≡C—, —C≡C≡CH₂—, —CH₂CH₂C(O)—, —CH₂C(O)CH₂—, —C(O)CH₂CH₂—, —CH₂CH₂S(O)₂—, —CH₂S(O)₂CH₂—, —S(O)₂CH₂CH₂—, —C(O)CH═CH—, —C(O)C≡C—, —CH₂CH₂O—, —CH₂OCH₂—, —OCH₂CH₂—, —CH₂CH₂S—, —CH₂SCH₂—, —SCH₂CH₂—, —C(O)CH₂O—, —OCH₂C(O)—, —CH₂C(O)O—, —C(O)CH₂S—, —SCH₂C(O)—, —CH₂C(O)S—, —OC(O)CH₂—, —C(O)OCH₂—, —CH₂OC(O)—, —SC(O)CH₂—, —C(O)SCH₂—, —CH₂SC(O)—, —CH₂CH₂NH—, —CH₂NHCH₂—, —NHCH₂CH₂—, —CH₂CH₂NMe-, —CH₂NMeCH₂—, —NMeCH₂CH₂—, —C(O)CH₂NH—, —NHCH₂C(O)—, —CH₂C(O)NH—, —NHC(O)CH₂—, —C(O)NHCH₂—, —CH₂NHC(O)—, 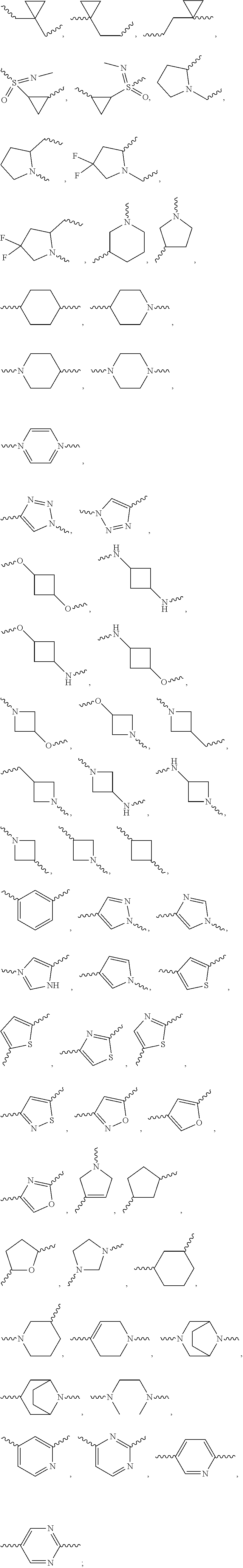
or two E moieties or two E′ moieties can be taken together to form —CH₂CH₂OCH₂CH₂—, —OCH₂CH₂CH₂CH₂—, —CH₂CH₂CH₂CH₂O—, 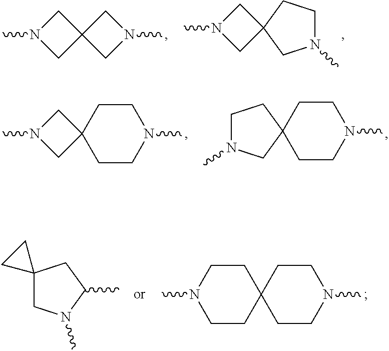
and one or more H atom(s) in the above groups can be substituted with D atom(s).

28. The compound of general formula (I), or the pharmaceutically acceptable salt, enantiomer, diastereomer, racemate, solvate, hydrate, polymorph, prodrug, or isotopic variant thereof, or mixture thereof according to claim 1, the compound is selected from:

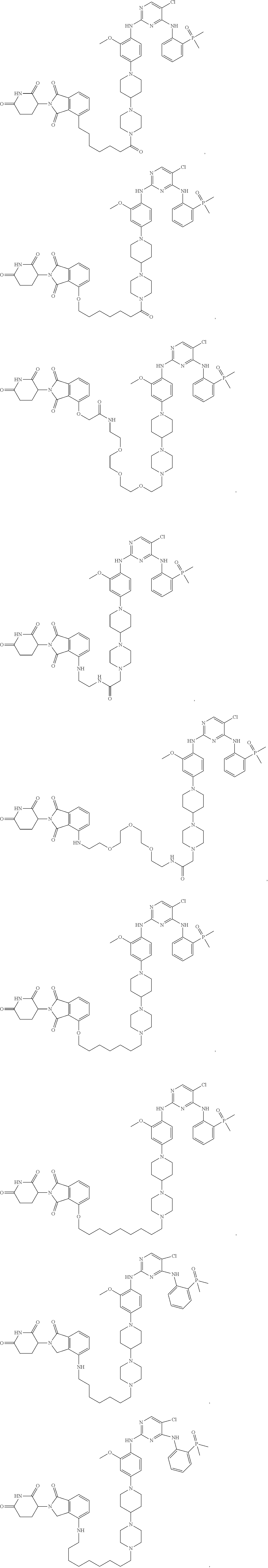

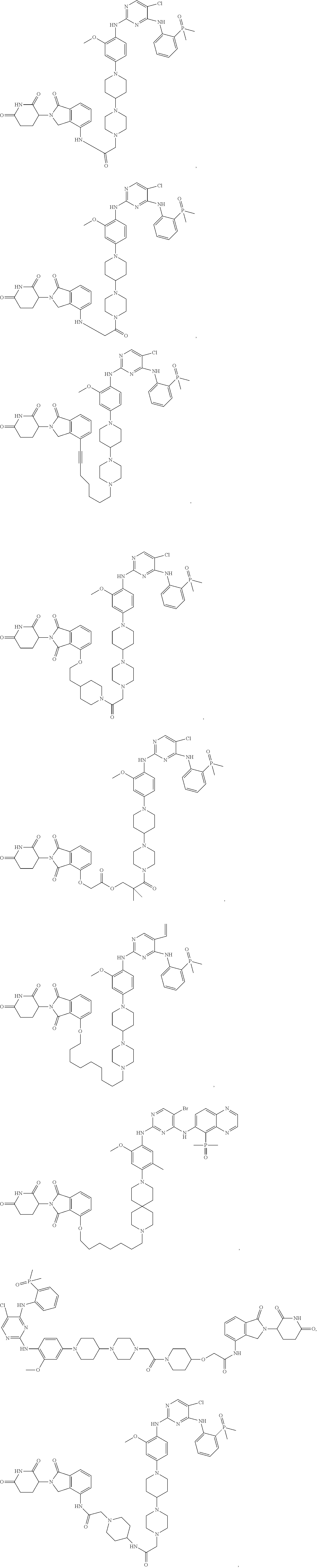

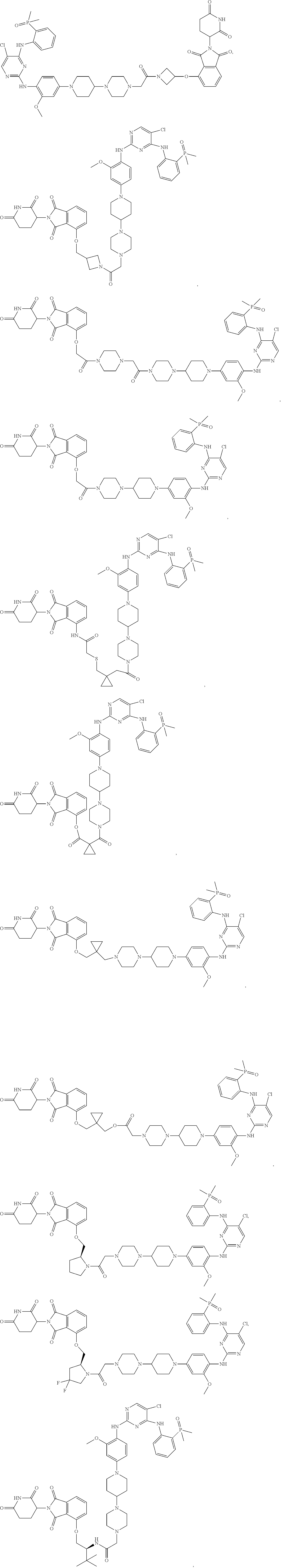

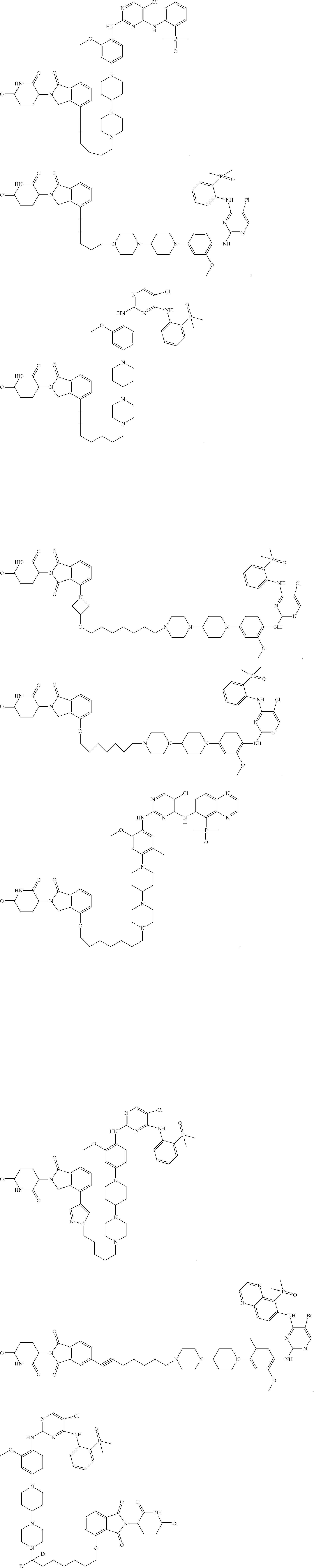

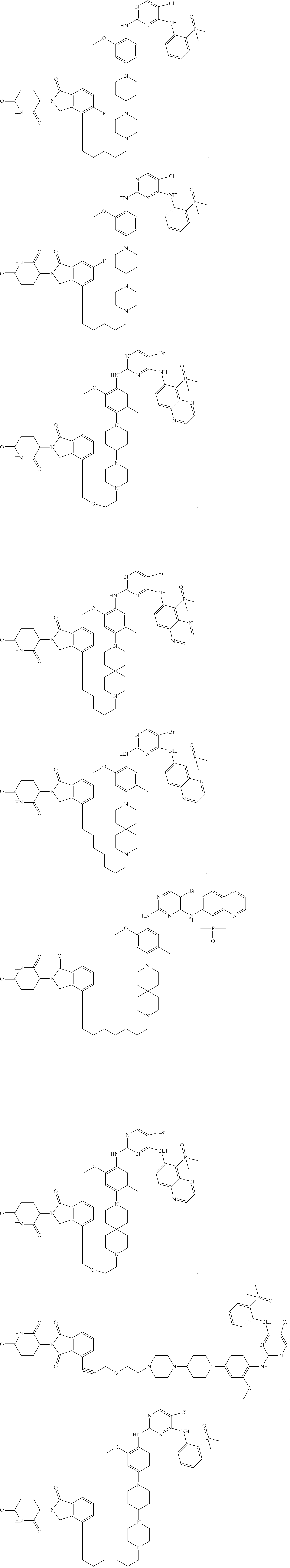

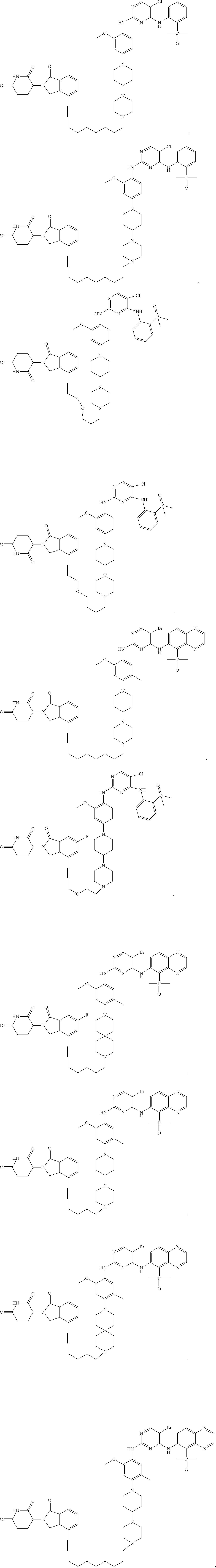

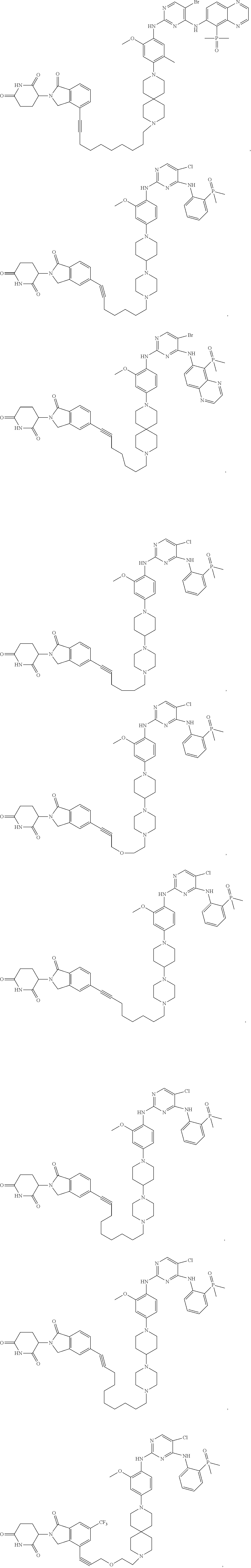

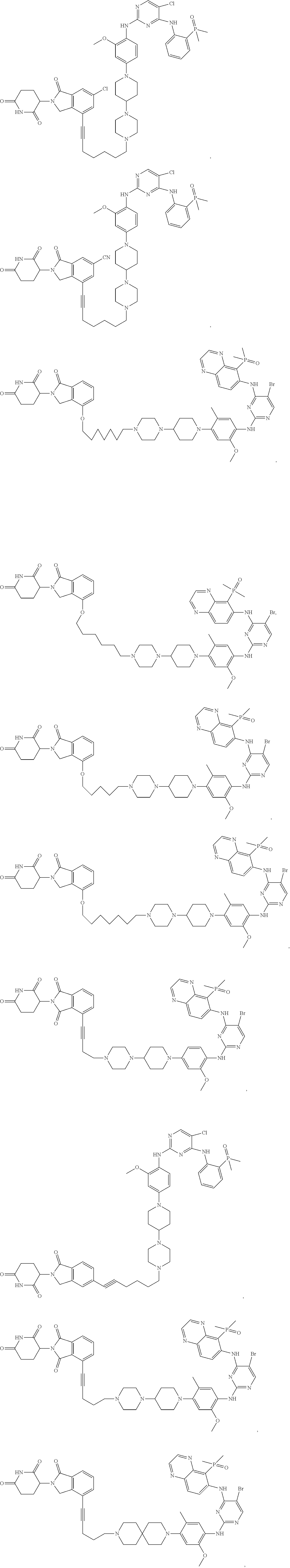

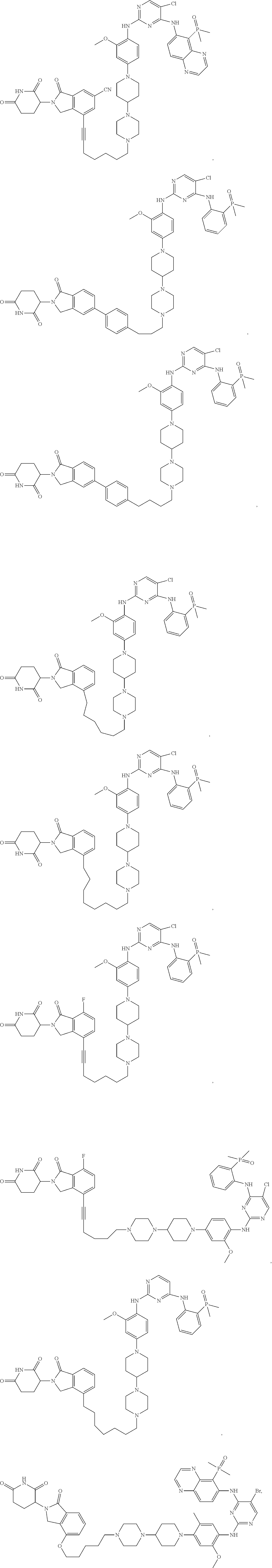

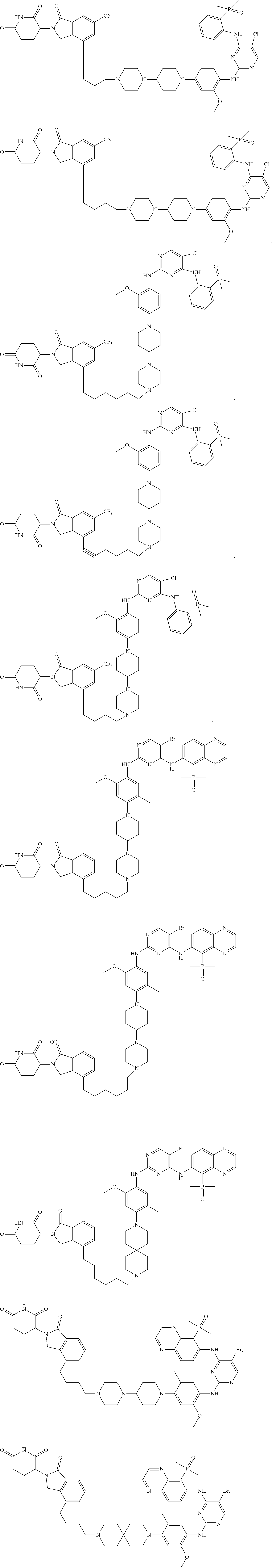

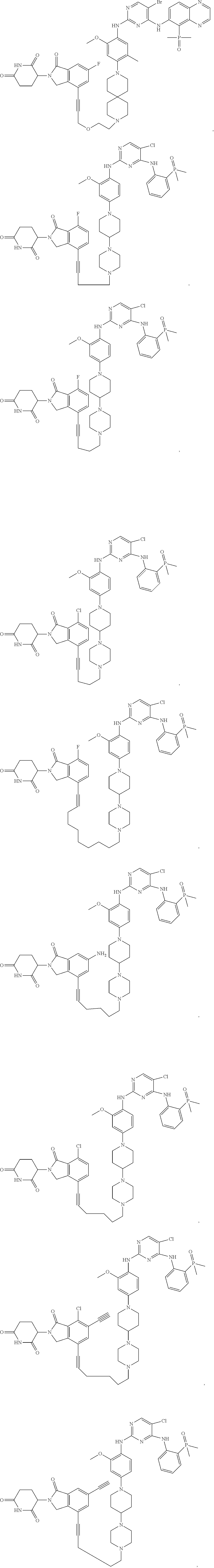

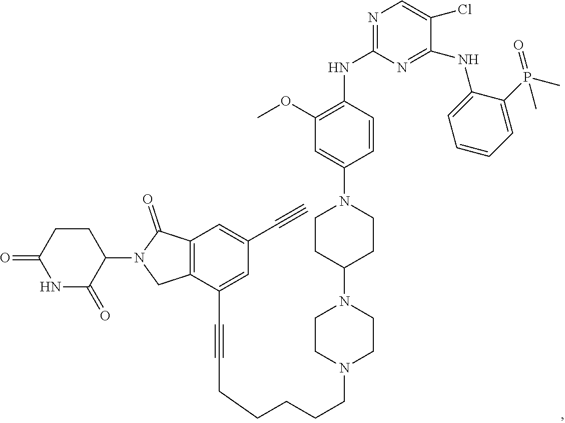

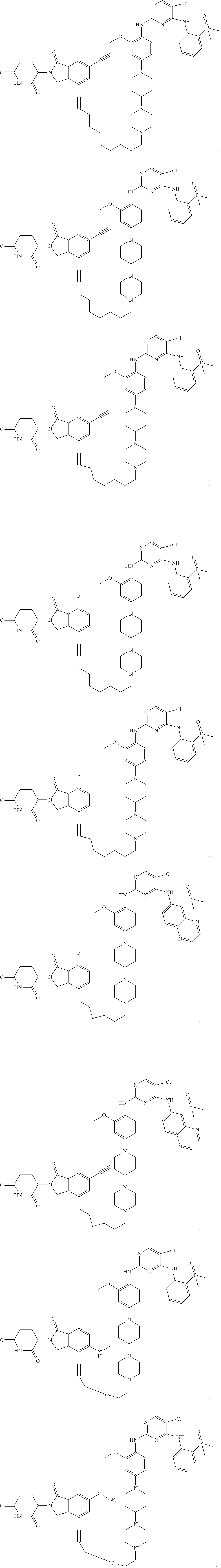

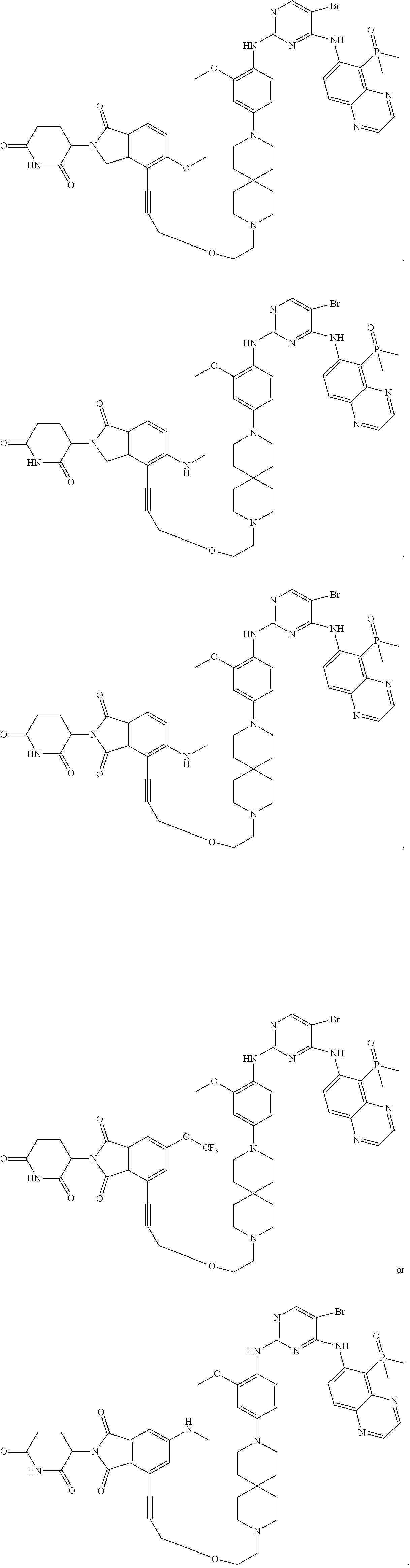

29. A pharmaceutical composition, comprising:
the compound, or the pharmaceutically acceptable salt, enantiomer, diastereomer, racemate, solvate, hydrate, polymorph, prodrug or isotopic variant thereof according to claim 1; and
pharmaceutically acceptable excipient (s);
alternatively, the pharmaceutical composition further comprises other therapeutic agent (s).

30. (canceled)

31. A method of treating and/or preventing diseases mediated by EGFR kinase and/or ALK kinase in a subject, which comprises administering to the subject the compound, or the pharmaceutically acceptable salt, enantiomer, diastereomer, racemate, solvate, hydrate, polymorph, prodrug or isotopic variant thereof according to claim 1.

32. (canceled)

33. The method according to claim 31, wherein the diseases mediated by EGFR kinase and/or ALK kinase include cancer, such as ovarian cancer, cervical cancer, colorectal cancer, breast cancer, pancreatic cancer, glioma, glioblastoma, melanoma, prostate cancer, leukemia, lymphoma, non-Hodgkin's lymphoma, gastric cancer, lung cancer, hepatocellular cancer, stomach cancer, gastrointestinal stromal tumor (GIST), thyroid cancer, cancer of bile duct, endometrial cancer, kidney cancer, anaplastic large cell lymphoma, acute myeloid leukemia (AML), multiple myeloma, melanoma, mesothelioma.
